(19) United States
(12) Patent Application Publication
(10) Pub. No.: US 2023/0043198 A1
(43) Pub. Date: Feb. 9, 2023

(54) MICROBIOTA METABOLITES THAT SHAPE HOST PHYSIOLOGY

(71) Applicant: Yale University, New Haven, CT (US)

(72) Inventors: Noah PALM, New Haven, CT (US); Aaron RING, New Haven, CT (US); Haiwei CHEN, New Haven, CT (US)

(73) Assignee: Yale University, New Haven, CT (US)

(21) Appl. No.: 17/312,260

(22) PCT Filed: Dec. 9, 2019
(86) PCT No.: PCT/US2019/065226

§ 371 Date: Jun. 9, 2021

Related U.S. Application Data
(60) Provisional application No. 62/777,480, filed on Dec. 10, 2018.

(51) Int. Cl. C12Q 1/6897; C12Q 1/689

(52) U.S. Cl. CPC C12Q 1/6897; C12Q 1/689

(57) ABSTRACT

Methods of identifying test compounds or mixtures of test compounds from microbiota that bind to a fusion protein, such as a G-protein coupled receptor, are described. Also described are methods for high throughput screening of microbiota metabolites that are capable of activating G-protein coupled receptors.

### CROSS-REFERENCE TO RELATED APPLICATIONS

This application claims priority to U.S. Provisional Application No. 62/777,480, filed Dec. 10, 2018, which application is herein incorporated by reference in its entirety.

### STATEMENT AS TO FEDERALLY SPONSORED RESEARCH

This invention was made with government support under grant AI123477 awarded by National Institutes of Health. The government has certain rights in the invention.

### BACKGROUND

The microbiota metabolome results from a complex web of interactions between multiple microbial species and strains, environmental inputs (e.g., diet), and host factors. Advanced metabolomic, metagenomic and functional genomic approaches have revealed that the human microbiota can produce tens of thousands of unique small molecules (Donia and Fischbach, 2015; Milshteyn et al., 2018; Nicholson et al., 2012). However, the sheer magnitude and complexity of the gut microbiota metabolome can create significant challenges for dissecting how intra- and inter-species microbial chemistries affect host physiology (Donia and Fischbach, 2015; Fischbach, 2018). Understanding of the potential effects of the microbiota metabolome on the human host remains in its infancy.

MAOIs were the first FDA approved antidepressants (Ramachandraih, 2011); however, they have fallen out of favor due to potentially fatal dietary and drug-drug interactions (Fiedorowicz, 2004). Nonetheless, MAOIs remain an important treatment option for patients with refractory depression and other psychiatric disorders (Fiedorowicz, 2004), as well as Parkinson's disease (Riederer and Laux, 2011).

Histamine is generated via decarboxylation of L-His (Tannase, 1985). Histamine is involved in inflammatory responses and can regulate gut physiological function.

### SUMMARY OF THE INVENTION

In one aspect is provided a method for determining if a test compound modulates an activity of a first protein in vivo, comprising

(a) contacting said compound with a cell comprising (i) a first nucleic acid molecule which encodes a first fusion protein comprising a first protein, a cleavage site for a protease, and a protein which activates transcription of a reporter gene in said cell, (ii) a second nucleic acid molecule which encodes a second fusion protein comprising a second protein which interacts with the first protein upon activation of the first protein and a protease or a fragment thereof capable of cleaving the protease cleavage site within the first fusion protein, and (iii) a third nucleic acid molecule which comprises a reporter gene, wherein said reporter gene is a barcode sequence operably linked to an element responsive to the protein which activates its transcription, and

(b) determining the level of transcription of said barcode sequence.

In some embodiments, the first nucleic acid molecule, the second nucleic acid molecule and the third nucleic acid molecule are clonally expressed to enable linkage of a specific receptor to an individual barcode. In some embodiments, the first nucleic acid molecule, the second nucleic acid molecule and the third nucleic acid molecule are clonally expressed through co-transfection. In some embodiments, the first nucleic acid molecule, the second nucleic acid molecule and the third nucleic acid molecule are clonally expressed through stable expression. In some embodiments, the barcode sequence comprises from 4 to 50 bases.

In some embodiments, the method further comprises

(c) concluding that the test compound activates the first protein if the level of transcription of said barcode sequence is increased in the presence of the test compound as compared to a control where no test compound is present.

In some embodiments, the method further comprises

(c) concluding that the test compound activates the first protein if the level of transcription of said barcode sequence is increased in the presence of the test compound as compared to an untreated cell.

In some embodiments, the method further comprises

(c) concluding that the test compound activates the first protein if the level of transcription of said barcode sequence is increased in the presence of the test compound as compared to a cell treated with a single dose of an agonist of the first protein in combination with an antagonist of the first protein.

In some embodiments, the first protein is a transmembrane protein. In some embodiments, the first protein is a G-protein coupled receptor (GPCR). In some embodiments, the GPCR is a non-olfactory GPCR. In some embodiments, the GPCR is an orphan GPCR. In some embodiments, the GPCR is a human GPCR.

In various embodiments, the protein which activates transcription of the reporter gene in said cell is tTA, a cas9 fusion protein, gal4/VP16, the estrogen receptor, the androgen receptor, the mineralocorticoid receptor, or the glucocorticoid receptor. In some embodiments, the cas9 fusion protein is cas9-vp64.

In various embodiments, the protein which activates transcription of the reporter gene in said cell is tTA. In some embodiments, the second protein which interacts with the first protein upon activation of the first protein is β-arrestin, a G-protein receptor kinase (GRK), or G-alpha. In some embodiments, the second protein is β-arrestin. In some embodiments, the protease is a Tobacco Etch Virus nuclear inclusion A protease (TEV protease). In some embodiments, the cell is a non-adherent mammalian cell. In some embodiments, the cell is Expi 293 T cell, Jurkat, Hela T4, raji, ramos, cho-s, or thp1 cells.

In various embodiments, the level of transcription of said barcode sequence is determined by sequencing of cDNA. In some embodiments, the cDNA is produced by reverse transcription of mRNA isolated from the cell using a polydT primer. In some embodiments, the test compound is a metabolite produced by a bacterial taxon contained within a microbiota of a subject. In some embodiments, the test compound is a metabolite produced by a bacterial strain contained within a microbiota of a subject. In some embodiments, the microbiota is a gastrointestinal (GI) microbiota. In some embodiments, the bacterial strain is clonally arrayed and cultured in vitro. In some embodiments, the method further comprises identifying the bacterial strain. In some embodiments, the bacterial strain is identified using 16S rRNA gene sequencing or whole genome sequencing. In some embodiments, the subject is human.

In some embodiments, the method is conducted in a high-throughput format, comprising:

(i) transfecting or transducing a plurality of cells separated into individual wells of a multi-well plate with the first, second and third nucleic acid molecules so that each transfected or transduced cell has a specific combination of the first protein and the barcode sequence;
(ii) mixing the transfected cells;
(iii) rearraying mixed cells into individual wells of a multi-well plate;
(iv) exposing the rearrayed cell mixtures to one or more test compounds;
(v) sequencing the barcodes, and
(vi) determining which barcode sequences are increased in the presence of the test compound(s) as compared to a control where no test compound(s) is present.

In some embodiments, the method is conducted in a high-throughput format, comprising:

(i) transfecting or transducing a plurality of cells that stably express the second nucleic acid molecule (Barr-TEV) with the first and third nucleic acid molecules (the receptor and barcode);

(ii) mixing the transfected cells;

(iii) rearraying mixed cells into individual wells of a multi-well plate;

(iv) exposing the rearrayed cell mixtures to one or more test compounds;

(v) sequencing the barcodes, and

(vi) determining which barcode sequences are increased in the presence of the test compound(s) as compared to a control where no test compound(s) is present.

In some embodiments, the first nucleic acid molecule encodes a GPCR, the second nucleic acid molecule encodes Barr-TEV, and the third nucleic acid molecule comprises a barcode.

In another aspect is provided a method for high-throughput screening of microbiota metabolites capable of modulating the activity of a plurality of G-protein coupled receptors (GPCRs), the method comprising:

a) providing a plurality of non-adherent mammalian cells, wherein each cell comprises (i) a first nucleic acid molecule encoding a first fusion protein comprising a GPCR linked to the transcription factor tTA via a cleavage site for Tobacco Etch Virus nuclear inclusion A protease (TEV protease), (ii) a second nucleic acid molecule encoding a second fusion protein comprising 3-arrestin and TEV protease configured to cleave the TEV protease site on the first fusion protein, and (iii) a third nucleic acid molecule comprising a barcode sequence operably linked to a promoter specifically activated by the tTA transcription factor, wherein each barcode sequence is thus specifically linked to an individual GPCR;

b) contacting the plurality of cells with one or more microbiota metabolites or one or more compounds;

c) sequencing the barcodes; and

d) determining which barcode sequence transcription levels are increased or decreased in the presence of the metabolites as compared to a control where no metabolites are present.

In some embodiments, the GPCR is a non-olfactory GPCR. In some embodiments, the GPCR is an orphan GPCR. In some embodiments, the GPCR is a human GPCR. In some embodiments, the cell is Expi 293 T cell. In some embodiments, the level of transcription of said barcode sequence is determined by sequencing of cDNA. In some embodiments, the cDNA is produced by reverse transcription of RNA isolated from the cell. In some embodiments, the microbiota is a gastrointestinal (GI) microbiota. In some embodiments, the method further comprises identifying a bacterial strain producing the specific metabolite which activates the specific GPCR. In some embodiments, the bacterial strain is identified using 16S rRNA sequencing.

In another aspect is provided a method of preventing or treating monoamine oxidase inhibitor (MAOI)-induced toxicity in a subject, the method comprising administering an antibiotic effective to target a bacterial strain comprising a nucleic acid sequence encoding a phenethylamine production gene. In yet another aspect is provided a method of preventing or treating MAOI-induced toxicity in a subject comprising administering an antibiotic effective to target Morganella spp.

In another aspect is provided a method of treating a disease or condition caused by decreased MAO activity in a subject, the method comprising administering an antibiotic effective to target an organism comprising a nucleic acid sequence encoding a phenethylamine production gene.

In some embodiments, the organism is a bacterial strain. In some embodiments of the above aspects, the bacterial strain produces phenethylamine. In some embodiments, the bacterial strain secretes phenethylamine. In some embodiments, the disease is Brunner syndrome. In some embodiments, the condition is autism or anti-social behavior. In some embodiments, the subject expresses a MAOA-L variant. In some embodiments, the antibiotic is effective to target Morganella morganii. In some embodiments, the antibiotic is cefepime, piperacillin, tazobactam, ceftazidime, cefotaxime, ceftibuten, meropenem, doripenem, ertapenem, a fluoroquinolone, or an aminoglycoside.

In another aspect is provided a method of treating depression in a subject comprising administering a bacterial strain comprising a nucleic acid sequence encoding a phenethylamine production gene. In some embodiments, the method further comprises administering an anti-depressant to the subject. In some embodiments, the anti-depressant is an MAOI. In some embodiments, the bacterial strain produces phenethylamine.

In another aspect is provided a method for evaluating potential toxicity of a monoamine oxidase inhibitor (MAOI) in a subject, the method comprising

a) obtaining a gastrointestinal microbiota sample from the subject, and
b) assaying the sample for the presence of a bacterial taxon or bacterial strain comprising a phenethylamine production gene.

In some embodiments, the amount of a phenethylamine producing enzyme exceeds a defined fraction of the enzymes produced by the microbiota. In some embodiments, the gastrointestinal microbiota sample is a fecal sample.

In another aspect is provided a method for evaluating potential efficacy of an MAOI in a subject, the method comprising

a) taking a fecal sample or a sample from the gastrointestinal tract of the subject, and

b) assaying for the presence of a bacterial strain comprising a nucleic acid sequence encoding a phenethylamine production gene.

In some embodiments, the method further comprises assaying for the amount of the bacterial strain present in the gastrointestinal tract. In some embodiments, the method further comprises treating the subject with an MAOI. In some embodiments, the method further comprises adjusting or determining the dosage of the MAOI based on the presence and/or amount of the bacterial strain present. In some embodiments, the bacterial strain is a bacterium of Morganella spp. In some embodiments, the bacterial strain is Morganella morganii.

In another aspect is provided a method of preventing or treating histamine-induced gastrointestinal disease in a subject, the method comprising administering one or more antibiotics effective to target a bacterial strain comprising a nucleic acid sequence encoding a histamine production gene. In some embodiments, the histamine production gene is a histidine decarboxylase. In some embodiments, the abundance of the histidine decarboxylase is higher in a patient with Crohn's disease as compared to a subject without inflammatory bowel disease. In some embodiments, the abundance of the histidine decarboxylase is higher in a patient with ulcerative colitis as compared to a subject without inflammatory bowel disease. In some embodiments, the bacterial strain is L. reuteri or a bacterium of Morganella spp. In some embodiments, the gastrointestinal disease is diarrhea.

In another aspect is provided a method of preventing or treating an allergy in a subject, the method comprising administering one or more antibiotics effective to target a bacterial strain comprising a nucleic acid sequence encoding a histamine production gene. In some embodiments, the bacterial strain is L. reuteri or a bacterium of Morganella spp.

In another aspect is provided a method of preventing or treating asthma in a subject, the method comprising administering one or more antibiotics effective to target a bacterial strain comprising a nucleic acid sequence encoding a histamine production gene. In some embodiments, the bacterial strain is L. reuteri or a bacterium of Morganella spp. In some embodiments, the antibiotic is cefepime, piperacillin, tazobactam, ceftazidime, cefotaxime, ceftibuten, meropenem, doripenem, ertapenem, a fluoroquinolone, or an aminoglycoside. In some embodiments, the method further comprises administering a histidine decarboxylase inhibitor. In some embodiments, the histidine decarboxylase inhibitor is rugosin D, rugosin A methyl ester, tellimagrandin II, rugosin A, pinocembrin, α-fluoromethylhistidine, brocresine, lecanoric acid, 2-hydroxy-5-carbomethoxybenzyloxyamine, and aminooxy analogs of histamine.

In another aspect is provided a method for evaluating potential effectiveness of an antibiotic to treat a gastrointestinal condition or disease, allergy or asthma in a subject, the method comprising

a) taking a fecal sample or a sample from the gastrointestinal tract of the subject, and

b) assaying for the presence of a bacterial strain comprising a nucleic acid sequence encoding a histamine production gene.

In some embodiments, the method further comprises assaying for the amount of the bacterial strain present in the gastrointestinal tract. In some embodiments, the method further comprises treating the subject with one or more antibiotics. In some embodiments, the antibiotic is cefepime, piperacillin, tazobactam, ceftazidime, cefotaxime, ceftibuten, meropenem, doripenem, ertapenem, a fluoroquinolone, or an aminoglycoside. In some embodiments, the method further comprises adjusting or determining the dosage of the antibiotic based on the presence and/or amount of the bacterial strain present. In some embodiments, the bacterial strain is a bacterium of Morganella spp. In some embodiments, the bacterial strain is Morganella morganii.

In another aspect is provided a method of preventing or treating a disease or condition resulting from production of phenethylamine, the method comprising administering one or more antibiotics effective to target a bacterial strain producing L-phenylalanine. In yet another aspect is provided a method of preventing or treating phenylketonuria (PKU) in a subject, the method comprising administering one or more antibiotics effective to target a bacterial strain producing L-phenylalanine.

In another aspect is provided a method of preventing or treating a disease or condition resulting from production of phenethylamine, the method comprising administering a Shikimate pathway inhibitor or an antagonist of aromatic L-amino acid decarboxylase. In yet another aspect is provided a method of preventing or treating phenylketonuria (PKU) in a subject, the method comprising administering a Shikimate pathway inhibitor or an antagonist of aromatic L-amino acid decarboxylase.

In some embodiments, the antagonist is carbidopa, benserazide, methyldopa, 3′,4′,5,7-Tetrahydroxy-8-methoxyisoflavone (DFMD), 3-hydroxybenzylhydrazine, 3-amino-1-methyl-5H-pyrido[4,3-b]indole (Trp-P-2), or α-difluoromethyl DOPA. In some embodiments, the bacterial strain is B. thetaiotaomicron. In some embodiments, the bacterial strain is a strain C34 of B. thetaiotaomicron.

In some embodiments, the antibiotic is ampicillin, clavulanate, tazobactam, a cephamycin, ticarcillin, piperacillin, a cephalosporin, a carbapenem, clindamycin, lincomycin, chloramphenicol, a nitroimidazole, a fluoroquinolone.

In some embodiments, the antibiotic comprises (a) a combination of ampicillin and sulbactam, (b) a combination of ticarcillin and clavulanate, or (c) a combination of piperacillin and tazobactam.

In various embodiments of the above, the method further comprises administering a probiotic composition. In some embodiments, the probiotic composition is administered after antibiotic administration.

In some embodiments, the administration of the antibiotic and the administration of the probiotic composition are repeated in a cycle.

In another aspect is provided a kit for evaluating potential toxicity of an MAOI in a subject, the kit comprising a nucleic acid, antibody, or other reagent capable of binding specifically to a nucleotide or protein expressed by a bacterial strain, wherein the bacterial strain comprises a nucleic acid sequence encoding a phenethylamine production gene. In some embodiments, the bacterial strain is a bacterium of the Morganella spp. In some embodiments, the bacterial strain is Morganella morganii.

In another aspect is provided a kit for evaluating potential effectiveness of an antibiotic to treat a gastrointestinal condition or disease, allergy or asthma in a subject, the kit comprising a nucleic acid, antibody, or other reagent capable of binding specifically to a nucleotide or protein expressed by a bacterial strain, wherein the bacterial strain comprises a nucleic acid sequence encoding a histamine production gene. In some embodiments, the bacterial strain is L. reuteri or a bacterium of Morganella spp.

### BRIEF DESCRIPTION OF THE DRAWINGS

FIGS. 1A-1C are diagrams showing PRESTO-Salsa, a novel high-throughput technology to screen metabolite mixtures against hundreds of GPCRs in parallel in a single tube. FIG. 1A is a diagram depicting the PRESTO-Tango technology. Briefly, upon ligand binding to a GPCR, recruitment of a Beta-arrestin-Tobacco Etch Virus nuclear inclusion endopeptidase (Barr-TEV) fusion triggers release of the transcription factor tTA and production of luciferase. FIG. 1B shows modification of the PRESTO-Tango assay to link GPCR activation to transcription of a specific nucleic acid barcode rather than production of luciferase. FIG. 1C shows that by pooling cells that clonally express specific GPCR-barcode pairs, Presto-Salsa enables simultaneous evaluation of 300+ GPCRs in a single tube via NGS. In this example, the in vitro cultured bacterial strain is producing a ligand for ‘GPCR2’ which leads to increased transcription of the corresponding GPCR2 barcode.

FIG. 2 is a heatmap showing that PRESTO-Salsa accurately detects GPCR activation via barcode counting in pooled libraries. Activation of pooled Expi293 cells expressing GPCR-barcode pairs were stimulated with known GPCR ligands and activation was determined via next-generation sequencing and barcode counting.

FIG. 3 is a diagram showing the PRESTO-Tango experiment performed on 144 unique human gut bacteria spanning five phyla, nine classes, eleven orders, and twenty families; these bacteria were isolated from fecal samples from 11 inflammatory bowel disease patients via high-throughput anaerobic culturomics and identified via massively barcoded 16S rRNA gene sequencing. Each isolate was grown in monoculture in a medium specialized for the cultivation of human gut microbes (gut microbiota medium). Supernatants from individual bacterial monocultures were screened against the near-complete non-olfactory GPCRome (314 conventional GPCRs) using the high-throughput assay Parallel Receptor-ome Expression and Screening via Transcriptional Output-Tango (PRESTO-Tango).

FIG. 4 is a chart showing GPCR activation by metabolomes from 144 bacterial strains isolated from the human gut microbiota. Data is displayed on a hierarchical tree of GPCRs organized by class, ligand type, and receptor family. Shading intensity represents the maximum magnitude of activation (log 2) over background across the entire data set, i.e., the maximum activation of a given GPCR by any microbial metabolome in our collection.

FIG. 5A is a diagram showing activation of aminergic GPCRs by 144 human gut bacteria as measured by PRESTO-Tango. Screening results are displayed on a phylogenetic tree of aminergic GPCRs. The 5-HT receptors are 5HT1A, 5HT1B, 5HT1D, 5HT1E, 5HT1F, 5HT7R, 5HT5A, 5HT2B, 5HT2C, 5HT2A, and 5HT6R. The acetylcholine receptors are ACM2, ACM4, ACM3, ACM1, and ACM5. The adernoceptors are ADRB3, ADRB2, ADRB1, ADA2A, ADA2C, ADA2B, ADA1D, ADA1A, and ADA1B. The dopamine receptors are DRD1, DRD5, DRD3, DRD2, and DRD4. The histamine receptors are HRH2, HRH1, HRH3 AND HRH4. The trace amine receptor is TAAR1. Shading intensity represents magnitude of activation over media alone and radii of the circles represents the number of bacteria that activated a given GPCR by more than two-fold over media alone.

FIG. 5B is a heatmap depicting the activation of aminergic GPCRs by metabolites from 144 human gut bacteria as measured by PRESTO-Tango. Fold induction over stimulation with bacterial media alone is depicted on a log 2 scale.

FIG. 5C shows bar charts indicating activation of DRD2-4 and HRH2-4 by select species and strains as measured by PRESTO-Tango (n=3 technical replicates per isolate).

FIG. 5D is a bar chart showing quantification of dopamine, phenethylamine and tyramine production by M. morganii. Supernatants from 24-hour cultures of M. morganii in gut microbiota medium were analyzed by Electron Spray Ionization-Triple Quadrupole-Mass Spectrometry (ESI-QQQ-MS) and compared to those of media controls.

FIG. 5E is a plot showing quantification of histamine production by 144 isolates of human gut bacteria. All bacteria were grown in gut microbiota media for 48 hours and then supernatants were probed for histamine production via ELISA.

FIG. 5F shows mass spectrometric traces of metabolite production by M. morganii, which can directly convert L-Phe and L-His into phenethylamine and histamine, respectively. However, no conversion of L-Tyr to tyramine or L-DOPA to dopamine was observed. M. morganii was cultured in minimal medium (MM) with or without additional L-Phe, L-His, L-Tyr or L-DOPA for 48 hours. Metabolite production was analyzed by Liquid Chromatography-Mass Spectrometry (LC-MS).

FIG. 5G is a heatmap showing M. morganii-derived phenethylamine and histamine activate DRD2-4 and HRH2-4, respectively. M. morganii were cultured as described in F and supernatants were screened for activity against DRDs and HRHs by PRESTO-Tango.

FIGS. 6A and 6B show bar charts indicating that diverse human gut bacteria activate DRDs and HRHs. The charts show activation of DRD1-5 (FIG. 6A) and HRH1-4 (FIG. 6B) by supernatants from 144 human gut bacteria cultured in gut microbiota media (MM) as measured by PRESTO-Tango.

FIG. 7A is a chart showing the activation of CHRMs and DRDs by titrating doses of acetylcholine and dopamine as measured by PRESTO-Tango.

FIG. 7B is a heat map showing activation of GPCRs by defined GPCR ligands as measured by PRESTO-Tango. Activation is depicted on a log 2 scale as a heatmap of 314 GPCRs versus ligands.

FIGS. 8A-8F show identification of M. morganii-derived compounds that activate DRDs and HRHs. Data are representative of at least two independent experiments. FIG. 8A is an illustration of mammalian dopamine metabolism. FIG. 8B depicts data showing that phenethylamine and tyramine serve as selective DRD2/DRD3/DRD4 agonists. Activation of DRD1-5 by metabolites in the mammalian dopamine metabolism pathway was measured via PRESTO-Tango. FIG. 8C is a calibration curve for phenethylamine and tyramine on ESI-QQQ-MS instrument. FIG. 8D is a chart showing quantification of phenethylamine production by M. morganii strains via ESI-QQQ-MS/MS. FIG. 8E shows DRD1-5 activation by titrating doses of tyramine, dopamine and phenethylamine was measured by PRESTO-Tango (8D). FIG. 8F shows OD values for 24-hour cultures of M. morganii grown in minimal medium (MM) with or without L-Phe, L-Tyr, L-DOPA or L-His.

FIGS. 9A-9H show commensal-derived histamine promotes colon motility and M. morganii-derived phenethylamine combined with MAOI triggers lethal phenethylamine poisoning. Data in all panels are representative of at least two independent experiments.

FIG. 9A shows production of histamine by M. morganii and L. reuteri is enhanced by additional L-His. Four strains of L. reuteri and one strain of M. morganii were cultured in Gifu medium with or without supplemental L-His (n=3 per group) and histamine concentrations in the supernatants were measured by ELISA after 48 hours. The addition of L-His increased histamine production by L. reuteri C93, C94 and M. morganii C135 (all histamine producers), but not L. reuteri C88 and C89. FIG. 9B depicts an experimental design to test in vivo histamine production and the effects of histamine-producing bacteria on colon motility.

FIG. 9C shows bar charts of results in which groups of germ-free C57Bl/6 mice were colonized with mock communities of 9 or 10 phylogenetically diverse human gut bacteria (Mock Community A or B) or monocolonized with M. morganii, L. reuteri C88 or C93. Mice were fed a conventional diet with or without administration of 1% L-His ad libitum. Histamine concentrations in cecal and colonic extracts and feces were measured via ELISA. FIG. 9D is a bar chart showing M. morganii- or L. reuteri C93-derived histamine enhances colon motility. Fecal output for mice treated as described in B were measured by counting the number of fecal pellets produced by a single mouse in one hour.

FIG. 9E is a trace showing that M. morganii produces phenethylamine in vivo. Germ-free mice were colonized with M. morganii and treated with or without the MAOI phenelzine. Phenethylamine concentration in colonic extracts was examined using ESI-QQQ-MS/MS. In the graph of FIG. 9F, mice colonized with M. morganii exhibit lethal phenethylamine poisoning after treatment with the MAOI phenelzine. Germ-free C57Bl/6 mice were monocolonized with M. morganii for one week before treatment with phenelzine in the drinking water. Survival is depicted on a Kaplan-Meier curve. n=4 mice per group. In FIGS. 9G and 9H, M. morganii-colonized mice treated with phenelzine accumulated phenethylamine in the cecum, colon, serum and brain. M. morganii and B. theta monocolonized C57Bl/6 mice were treated with or without the MAOI phenylzine in the drinking water. Phenethylamine was detected in the cecum, colon, serum or brain via ESI-QQQ-MS (9G) or DRD2 PRESTO-Tango (9H).

FIGS. 10A-10F show M. morganii localization and production and accumulation of systemic phenethylamine in vivo, as related to FIGS. 9A-9H. Data are representative of at least two independent experiments. In FIG. 10A, groups of germ-free C57Bl/6 mice were colonized with mock communities of 9 or 10 phylogenetically diverse human gut bacteria (Mock Community A or B) or monocolonized with M. morganii, L. reuteri C88 or C93. Mice were fed a conventional diet with or without administration of 1% L-His ad libitum. Histamine concentrations in serum were measured via ELISA. FIGS. 10B-10D show M. morganii primarily inhabits the cecum and colon. Germ-free mice were colonized with mock communities of 9 or 10 phylogenetically diverse gut microbes (Mock community A and B, respectively) with or without M. morganii. CFUs from M. morganii can be distinguished from other bacteria based on their purple halos when plated on modified Niven's agar. Gastric, small intestinal, cecal and colonic contents from mice colonized with Mock communities A or B, and M. morganii, were plated on Modified Niven's agar to determine M. morganii colonization levels at various intestinal loci. FIG. 10E shows quantification of phenethylamine (PEA) in cecum, colon, and serum from mice monocolonized with M. morganii and treated with or without phenelzine (MAOI) via ESI-QQQ-MS/MS. FIG. 10F shows accumulation of phenethylamine (PEA) in sera and brains of mice monocolonized with M. morganii and treated with or without phenelzine (MAOI) as measured via ESI-QQQ-MS/MS.

FIGS. 11A-11G show that a unique strain of B. thetaiotaomicron C34 is a prolific producer of L-Phe and activates GPR56/AGRG1. Data in all Figures except for 11A, 11B, and 11E are representative of at least three independent experiments.

FIGS. 11A and 11B are diagrams showing that activation of orphan GPCRs by supernatants from 144 diverse human gut bacteria grown in gut microbiota medium (FIG. 11A) or Gifu (FIG. 11B) as measured by PRESTO-Tango. Screening results are displayed on a phylogenetic tree of orphan GPCRs that was constructed and visualized with equal branch lengths using gperdb.org, PHYLIP and jsPhyloSVG. The class A orphans are GPR21, GPR52, GP143, GPR32, GPR1, GPR152, MAS, MRGRF, MRGX2, MRGX4, MRGX1, MRGX3, MRGRD, MAS1L, MRGRE, MRGRG, GP182, GPR15, GPR25, GPR82, GPR34, GPR87, GP171, GP183, GP132, PSYR, GPR4, OGR1, GPR17, GPR174, GPR35, GPR20, GPR31, GPR88, MTR1L, GPR84, GP148, GP142, GPR19, GPR82, GPR27, GP172, GPR85, GP150, GPR6, GP146, GPR26, GPR78, GP135, GP161, GP101, GPR12, GPR3, GPR6, TAAP2, TAAP5, TAAP9, TAAP6, TAAP8, GP151, ETBR2, GPR37, GPR39, GPR75, GPR45, GPR63, GPR22, GP141, GP160, GP153, GP162, and GP149. The class C orphans are GP156, GP158, GPC6A, GPC5B, GPC5C, GPC5D, and RAI3. The ADGRA group is AGRA1, AGRA2, and AGRA3. The AGRGD group is AGRD1 and AGRD2. The ADGRG group is AGRG1, AGRG2, AGRG3, AGRG5 and AGRG6. The ADGRF group is AGRF1, AGRF2, AGRF3, AGRF4 and AGRF5. The other GPCR orphan is GP157. Shading intensities represent the magnitude of activation over media and radii of circles represent the number of bacteria that activated a given GPCR by more than two-fold. FIG. 11C depicts bar charts showing a single isolate C34 assigned to the species Bacteroides thetaiotaomicron activates GPR56/AGRG1 when cultured in gut microbiota medium (GMM) or Gifu medium. Activation of GPR56/AGRG1 by supernatants from 144 human gut isolates was measured via GPR56 PRESTO-Tango.

FIG. 11D is a bar chart showing that B. theta strain C34 uniquely activates GPR56/AGRG1. Activation of GPR56/AGRG1 by supernatants from diverse species and strains from the genera Bacteroides and Parabacteroides culture in GMM was measured via GPR56 PRESTO-Tango. FIG. 11E is a bar chart showing that B. theta C34-produced L-Phe activates GPR56/AGRG1. B. theta C34 supernatants were fractionated via reversed-phase HPLC and fractions were evaluated for activation of GPR56/AGRG1 via GPR56 PRESTO-Tango. The active fraction (F11) contained a primary constituent that was identified via LC-MS, HRMS-ESI-QTOF, NMR, and advanced Marfey's analyses as L-Phe. FIG. 11F is a graph showing that L-Phe preferentially activates the orphan receptor GPR56/AGRG1. Activation of GPR56/AGRG1 by titrating doses of pure L-Phe, L-Tyr, L-Trp, and L-His was measured via GPR56 PRESTO-Tango. FIG. 11G is a graph showing that the extracellular domain of GPR56/AGRG1 is indispensable for GPR56/AGRG1 activation by L-Phe. Activation of GPR56 or GPR56-ANT (a mutant lacking the extracellular domain) by titrating doses of L-Phe was measured via PRESTO-Tango.

FIGS. 12A-12D show the effect of different bacterial and culture media on bacterial growth and GPR56/AGRG1 activation, and structural characterization of B. theta C34 agonist L-Phe, as related to FIGS. 11A-11G. FIG. 12A shows OD₆₀₀ values of indicated Bacteroides and Parabacteroides strains cultured in gut microbiota medium (GMM) for 24 hours. FIG. 12B shows a ¹H NMR spectrum of active fraction 11 in MeOD revealed Phe as the major component. FIG. 12C shows the results of an advanced Marfey's analysis that verified the stereochemistry of Phe in fraction 11 to be L-Phe. D-Phe in the active fraction was not detected. FDAA is 1-fluoro-2,4-dinitrophenyl-5-L-alanine amide (Marfey's Reagent). FIG. 12D shows that L-Phe stereoselectively activates the orphan receptor GPR56/AGRG1. Activation of GPR56/AGRG1 by titrating doses of pure L-Phe, L-Tyr, L-Trp, L-His, D-Phe, D-Tyr, D-Trp, and D-His was measured via GPR56 PRESTO-Tango.

FIGS. 13A-13D illustrate data showing that L-Phe activates GPR97/AGRG3, a close relative of GPR56/AGRG1. Data are representative of at least three independent experiments. FIG. 13A is a heatmap showing that L-Phe activates GPR56/AGRG1 and GPR97/AGRG3. Activation of all orphan, adhesion and other potential amino acid-sensing GPCRs by L-Phe was evaluated via PRESTO-Tango. FIG. 13B is a graph showing that L-Phe specifically activates GPR97/AGRG3. Activation of GPR97/AGRG3 by titrating doses of L-Phe, L-Tyr, L-Trp, and L-His was measured via GPR97 PRESTO-Tango. FIG. 13C is a graph showing that the extracellular domain of GPR97/AGRG3 is indispensable for GPR97/AGRG3 activation by L-Phe. Activation of GPR97 or GPR97-ANT (a mutant lacking the extracellular domain) by titrating doses of L-Phe was measured via PRESTO-Tango. FIG. 13D is a phylogenetic tree showing that GPR56/AGRG1 and GPR97/AGRG3 are evolutionarily related. The phylogenetic tree for a subset of GPCRs, including all adhesion GPCRs, was constructed and visualized with equal branch lengths using gperdb.org, PHYLIP and jsPhyloSVG.

FIGS. 14A-14E depict data showing that active metabolic exchange between two commensals supports production of phenethylamine. The data are representative of at least two independent experiments. FIGS. 14A and 14B are data showing that B. theta C34 can directly synthesize L-Phe. L-Phe concentrations in supernatants from C34 grown in a minimal medium (SACC) lacking L-Phe were evaluated by LC-MS (14A) and quantitated by ESI-QQQ-MS/MS (14B). FIG. 14C is a graph showing that B. theta C34 produces L-Phe in vivo. Groups of germ-free C57Bl/6 mice fed a conventional diet or a defined diet lacking L-Phe were colonized with or without B. theta C34. Fecal L-Phe concentrations were measured by ESI-QQQ-MS/MS one week after colonization. FIG. 14D is a trace showing that M. morganii consumes B. theta C34-derived L-Phe to produce phenethylamine in vitro. B. theta C34 cultures were grown in SACC medium lacking L-Phe. Supernatants of C34 cultures were later incubated with M. morganii. ESI-QQQ-MS/MS traces of L-Phe and phenethylamine (PEA) levels in these cultures are depicted here. FIG. 14E is a graph showing that B. theta C34 and M. morganii can participate in active metabolic exchange to produce phenethylamine in vivo. Groups of germ-free C57Bl/6 mice were monocolonized with M. morganii or co-colonized with B. theta C34 and M. morganii, fed a diet lacking L-Phe and treated with the MAOI phenelzine. Activation of DRD2 by phenethylamine in cecal and colonic extracts was measured by PRESTO-Tango.

FIG. 15A is a graph showing that Crohn's disease patients display an increased presence and relative abundance of histidine decarboxylase genes in their gut microbiome. Metagenomic data from fecal samples collected from healthy controls (nonIBD), Crohn's disease (CD) and ulcerative colitis (UC) were downloaded from the NIH-funded Human Microbiome Project and analyzed for the presence and relative abundance of histidine decarboxylase genes. The number of total samples that contained detectable histidine decarboxylase genes and the number of patients that had detectable genes in any given sample are shown (all participants donated multiple samples at distinct timepoints).

FIG. 15B is a graph showing that Crohn's disease patients display an increased presence and relative abundance of histidine decarboxylase genes encoded by M. morganii (Morganella) in their gut microbiome. Metagenomic data from fecal samples collected from healthy controls (nonIBD), Crohn's disease (CD) and ulcerative colitis (UC) were downloaded from the NIH-funded Human Microbiome Project and analyzed for the presence and relative abundance of the M. morganii histidine decarboxylase gene. The number of total samples that contained detectable histidine decarboxylase genes and the number of patients that had detectable genes in any given sample are shown (all participants donated multiple samples at distinct timepoints).

FIG. 16 is a graph showing the results of an experiment in which a new ultra-highthroughput combinatorial GPCR screening technology PRESTO-Salsa enables simultaneous screening of hundreds of GPCRs in a single well of a 96 well plate. The data was generated according to the protocol described in Example 9.

### DETAILED DESCRIPTION OF THE INVENTION

In one aspect is provided a method allowing for parallel screening of multiple GPCRs (e.g., all 300 or more conventional GPCRs) in a single tube. Exemplary embodiments of this method are referred to herein as PRESTO-Salsa, in which instead of a luciferase reporter, reporter plasmids that encode unique nucleic acid barcodes are used.

Without wishing to be bound by theory, trillions of bacteria that constitutively colonize human intestines (gut microbiota) produce tens of thousands of unique small molecules that can potentially affect nearly all aspects of human physiology, from regulating immunity in the gut to shaping mood and behavior. However, this complexity can make it challenging to identify biologically relevant microbiota metabolites hidden in a sea of other chemicals. In other words, given the presence of tens of thousands of unique, unannotated metabolites in the microbiota metabolome, it can be difficult to decide which features should be prioritized for in-depth examination and characterization.

The inventors propose a revolutionary new approach to these problems. Without wishing to be bound by theory, the inventors describe host sensing of microbiota metabolites as a lens to illuminate the ‘dark matter’ that constitutes the majority of the bioactive microbiota metabolome. The inventors hypothesize that the hundreds of G-protein coupled receptors (GPCRs) encoded in the human genome can be used to identify novel bioactive microbiota metabolites from complex mixtures.

A new approach is described herein to investigate the bioactive microbiota metabolome where we used the sensing of microbiota metabolites by host GPCRs as a lens to illuminate bioactive metabolites produced by individual gut microbes. The examples described herein describe how the approach revealed a plethora of novel microbiota metabolite-GPCR interactions of potential physiological importance. For example, the inventors uncovered a diet-microbe-host axis that influences intestinal motility through the microbial production of histamine and a microbe-microbe-host axis that results in the production of the potent trace amine phenethylamine. Both of these axes can have profound effects on local and systemic host physiology. The functional profiling-based approach to understanding the contribution of the microbiota to human physiology described herein may be broadly applicable to understanding and illuminating diverse features of the bioactive microbiota metabolome.

An existing low-throughput GPCR-screening technology (PRESTO-Tango) was used by the inventors to identify metabolite mixtures from individual human gut bacteria that activated GPCRs involved in carcinogenesis, mood regulation, and immunity, as well as ‘orphan’ GPCRs whose natural ligands have evaded discovery for decades. However, because of inherent technical limitations, PRESTO-Tango can only be applied to a relatively small number of metabolite mixtures that are readily available in large quantities—e.g., metabolites produced by bacterial strains that can be cultured in vitro. PRESTO-Tango can thus only capture a small proportion of the overall bioactive microbiota metabolome, which normally results from complex in vivo interactions between multiple microbes, dietary compounds, and the host itself.

The inventors have leveraged recent developments in next-generation sequencing and nucleic acid barcode analyses to develop PRESTO-Salsa.

In one aspect a method is provided for high-throughput screening of microbiota metabolites capable of activating a plurality of G-protein coupled receptors (GPCRs). A plurality of non-adherent mammalian cells is provided. Each cell comprises (i) a first nucleic acid molecule encoding a first fusion protein comprising a GPCR linked to the transcription factor tTA via a cleavage site for Tobacco Etch Virus nuclear inclusion A protease (TEV protease), (ii) a second nucleic acid molecule encoding a second fusion protein comprising β-arrestin and TEV protease configured to cleave the TEV protease site on the first fusion protein, and (iii) a third nucleic acid molecule comprising a barcode sequence operably linked to a promoter specifically activated by the tTA transcription factor, wherein each barcode sequence is specific for an individual GPCR. The plurality of cells is contacted with a plurality of microbiota metabolites. The barcodes are sequenced. A determination is made as to which barcode sequences are increased in the presence of the metabolites as compared to a control where no metabolites are present.

Transcription of a barcode can indicate the activation of a GPCR. Without wishing to be bound by theory, when a GPCR is activated, the conformation of the GPCR changes such that a fusion protein comprising β-arrestin and TEV protease is recruited. The TEV protease then cleaves the protease site linking the tTA transcription factor and the GPCR so as to release the tTA transcription factor, which in turn drives transcription of a barcode operatively linked to the promoter.

A variety of GPCRs can be used. The GPCR may be a non-olfactory GPCR, an orphan GPCR, or a human GPCR. In preferred embodiments, the GPCR is one of the 314 conventional GPCRs described herein.

The microbiota metabolites may come from any number of samples, such as a fecal sample, or a sample from the gastrointestinal tract. The fecal sample or the sample from the gastrointestinal tract may be cultured and/or exposed to nutrients so as to enhance production of a particular microbiota metabolite. For example, additional L-Phe and L-His can be supplied to increase sensitivity of detection of M. morganii, which can directly convert L-Phe and L-His into phenethylamine and histamine, respectively. The sample may also be fractionated based on chemical properties, for example by reversed phase HPLC.

Without wishing to be bound by theory, the use of barcodes can greatly improve sensitivity of detection and allow for high-throughput screening. A larger number of cells expressing a wider variety of unique GPCR-barcode pairs can be used. Highly sensitive quantitative methods can be used to assay for the expression of all barcodes, e.g., as described in Example 1.

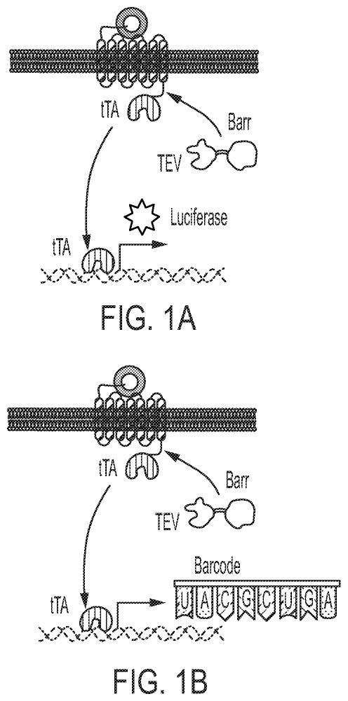

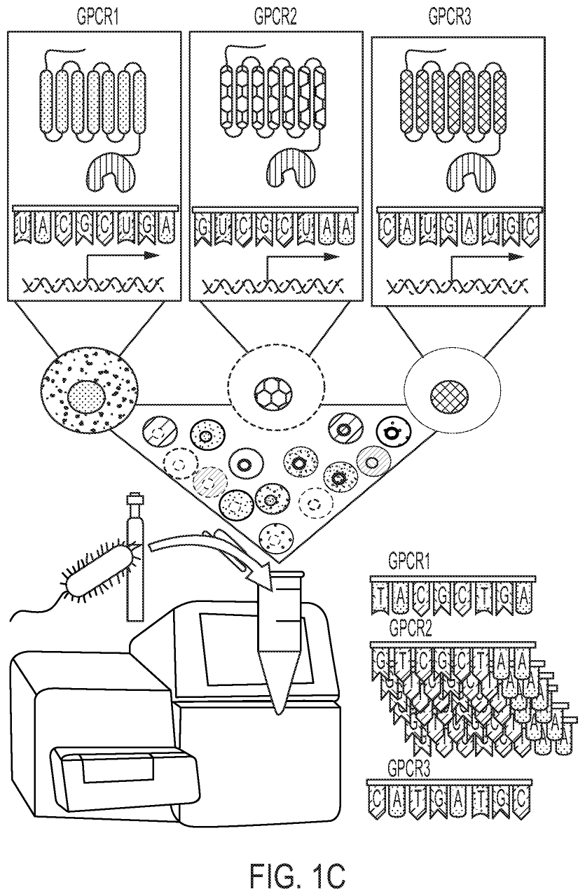

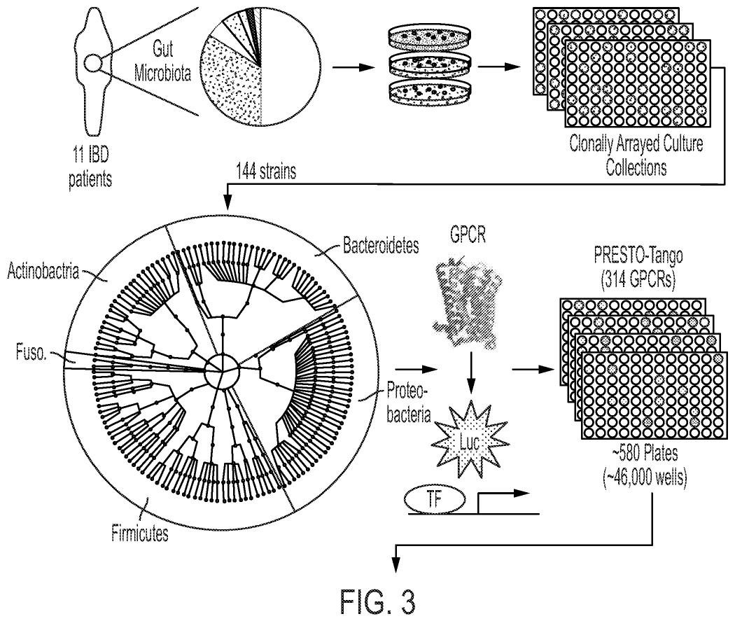

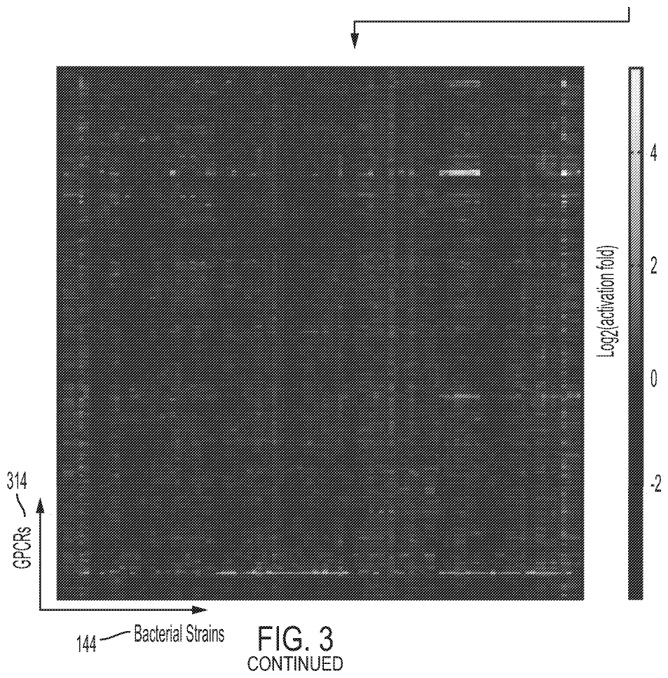

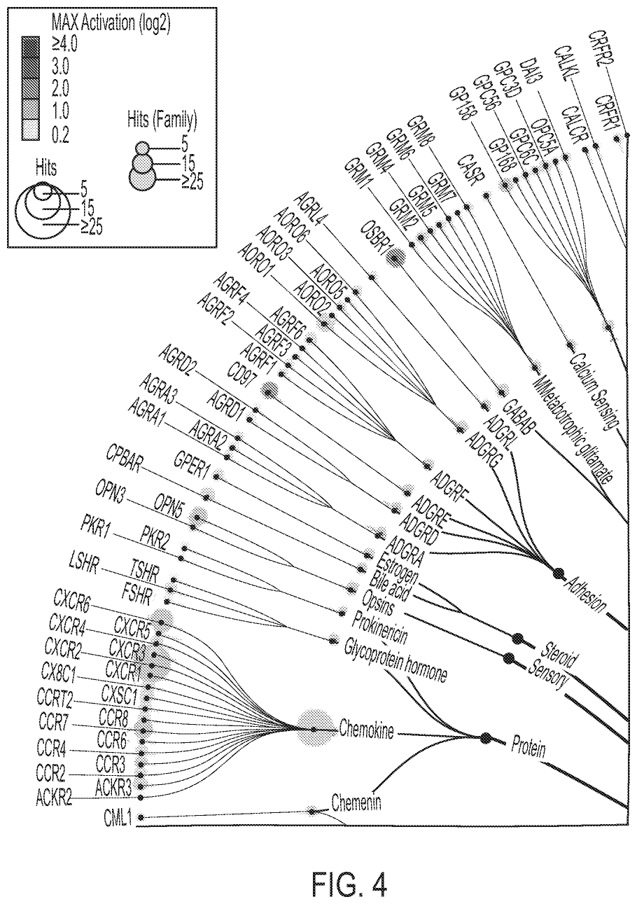

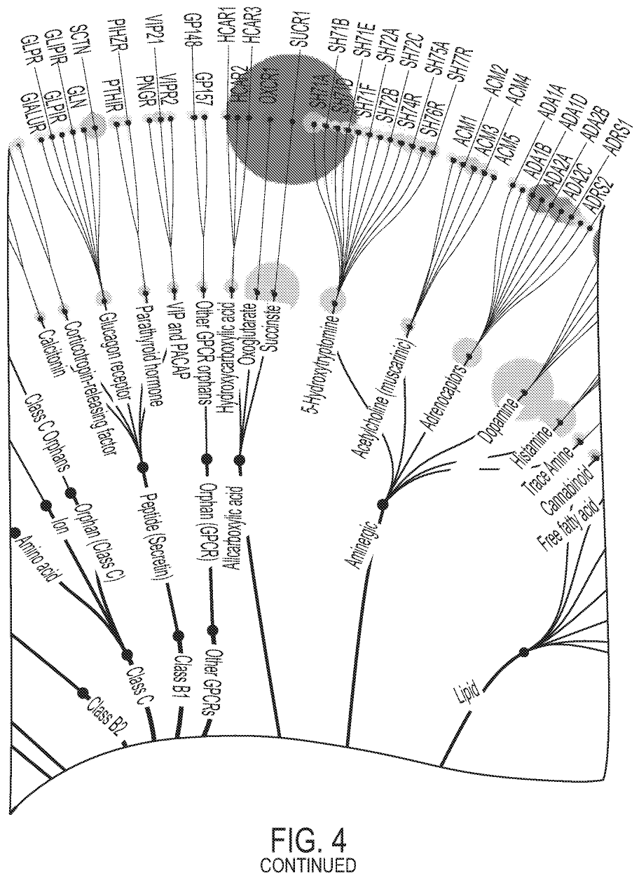

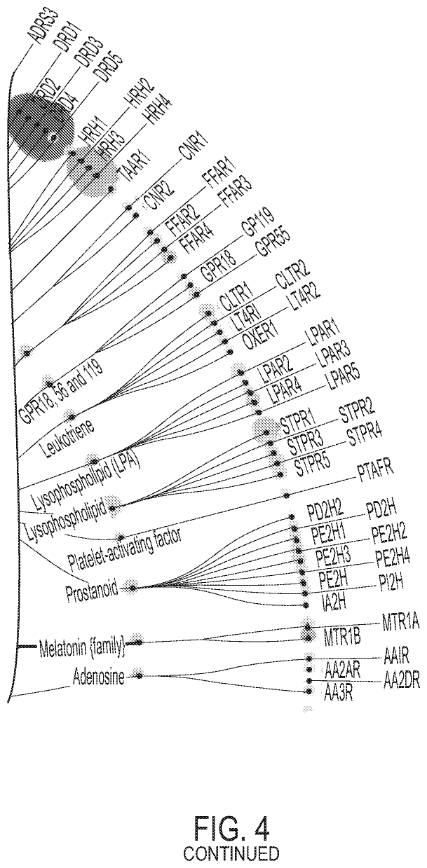

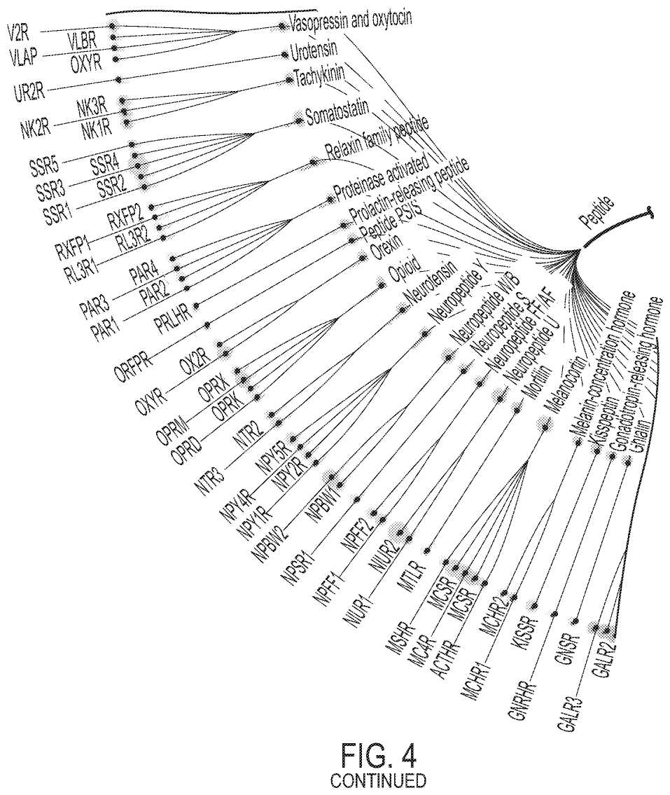

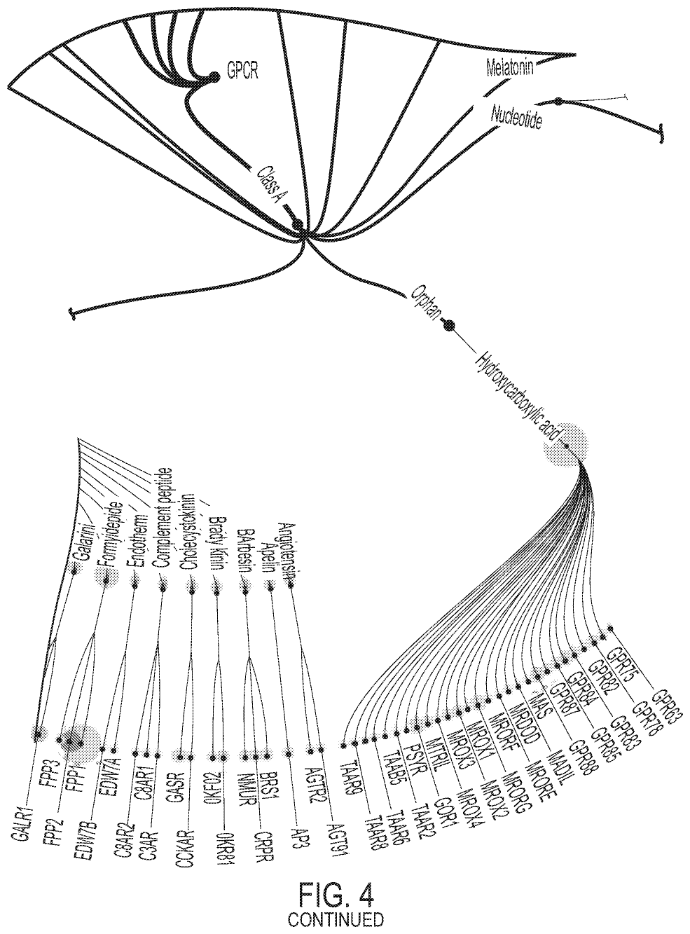

As shown in FIG. 1A, the traditional PRESTO-Tango approach connects GPCR activation to luciferase expression by linking Beta-arrestin recruitment following ligand binding to the release of a transcription factor (tTA) that is tethered to the GPCR of interest. The inventors modified this system by replacing the luciferase reporter with reporter plasmids that encode unique nucleic acid barcodes, as shown in FIG. 1B. The major novelty of PRESTO-Salsa is the use of transcription of unique barcodes to enable multiplexing of hundreds of GPCRs in a single well. This improves upon the PRESTO-Tango technology by increasing potential throughput by multiple orders of magnitude and allowing for examination of previous low-volume samples. PRESTO-Salsa was tested as shown in Example 1 and in FIGS. 3 and 4.

By pooling cells that express unique GPCR-barcode pairs and then quantifying the expression of specific barcodes after stimulation, activation or inhibition of hundreds of GPCRs in a single tube can be simultaneously screened for. See, FIG. 1C.

The method can further comprise identifying a bacterial strain producing the specific metabolite which activates the specific GPCR. Bacterial strains can be classified by 16S rRNA sequencing, for example, or by whole genome sequencing.

Individual bacterial strains can be isolated and cultured from a particular sample of interest, with metabolites from each cultured strain further tested in the various PRESTO-Salsa embodiments described herein. Alternatively, a bacterial strain can be added to a microbiota sample to assess the metabolites produced by the bacterial strain in the context of a given microbiome. The microbiota sample can then be cultured to allow the added bacterial strain to produce additional metabolites. A differential analysis of the barcodes produced by the PRESTO-Salsa method can be undertaken.

It was found that dozens of human gut bacteria from diverse phyla, families, species, and strains produced small molecules that activated various GPCRs, including both well-characterized GPCRs and orphan GPCRs. Patterns of metabolite production were observed that were largely predictable based on phylogeny, as well as strain-specific differences within a given species. Future studies will be necessary to determine when and why specific pathways are conserved or not in distinct species and strains. Metabolites resulting from core metabolic processes essential to a given microbe might be highly conserved, while metabolites involved in competitive processes may show considerable strain variation that could depend on the niche that is occupied and the need to compete with other bacterial species, non-bacterial microbes, or with the host. Regardless of the initial impetus for microbial metabolite expression, the data described herein support the concept that human-associated microbes exhibit impressive regulatory capabilities and are likely one of the richest sources of small molecules that impact human biology.

The high throughput of PRESTO-Salsa can allow for ability to easily examine biological or technical replicates, as well as perform dose response curves, for all samples. Finally, PRESTO-Salsa may be resistant to well-known off-target effects of certain chemicals on luciferase stability and thus may also reduce false positives.

PRESTO-Salsa provides a major technical advantage in that hundreds of receptors may be screened simultaneously in a single tube using small sample input volumes. PRESTO-Salsa is modular and flexible, and thus can be expanded to include other receptor families. Also, PRESTO-Salsa can easily be modified to simultaneously monitor multiple signaling outputs downstream of the same receptors (e.g., G-protein based signaling versus B-arrestin based signaling by GPCRs) to allow for identification of biased agonists and antagonists. PRESTO-Salsa enables screening of precious low-volume samples against hundreds of sensors while a similar sample volume used in PRESTO-Tango would only have been sufficient to examine the effects on one or two receptors. PRESTO-Salsa can provide for imminent scalability and automation. A major economic advantage of PRESTO-Salsa is that PRESTO-Salsa can enable simultaneous screening of hundreds of receptors in a single tube using low input volumes; prior technologies would require multiple plates of cells and a large input sample volume to accomplish this same goal. The use of next-gen sequencing as the final readout in PRESTO-Salsa is considerably cheaper than the prior Luciferase based screening (especially as next-gen sequencing continues to decline in cost). The cost of screening using PRESTO-Salsa may be lower than that of established assays by at least an order of magnitude.

The microbiota metabolome results from a complex web of interactions between multiple microbial species and strains, environmental inputs (e.g., diet), and host factors. Using a reductionist approach, the inventors discovered two bacterial isolates that traffic in the same small molecule: a unique strain of B. theta that is a prolific producer of L-Phe and M. morganii, which efficiently converts L-Phe into phenethylamine. The approaches described herein can reveal metabolic exchanges that may be missed when examining endpoint microbiota metabolomes produced by complex mixtures of microorganisms (e.g., complete gut microbial communities).

In various embodiments of the methods described herein, any GPCR agonists can be further tested in vivo, for example to examine the possibility that production of GPCR agonists by specific microbes would shape host physiology in vivo. The data in the examples show that histamine production by M. morganii or L. reuteri promotes increased colon motility and that feeding with exogenous histidine further increases microbial production of histamine and colonic motility. M. morganii monocolonized mice fed with histidine exhibited elevated levels of systemic histamine, indicating that microbiota-derived histamine may also shape systemic immune responses.

The inventors' studies also uncovered a specific Bacteroides strain that uniquely produces high levels of the essential amino acid L-Phe and revealed that L-Phe could activate the orphan GPCRs GPR56 and GPR97. GPR56 is highly expressed in the small intestine and human pancreatic islets (Amisten et al., 2013; Duner et al., 2016), and L-Phe concentrations in the jejunum can reach concentrations up to 2 mM after a meal (Adibi, 1973). Thus, GPR56 may act as a nutrient sensor to regulate digestion and satiety.

Although L-Phe levels in the serum usually are well below the levels necessary to activate GPR56/97, patients with phenylketonuria (PKU) who cannot degrade L-Phe exhibit serum concentrations of L-Phe higher than 1 mM (Williams, 2008). Thus, activation of GPR56 and/or GPR97 may contribute to some of the symptoms of PKU. Notably, GPR56 is highly expressed in neural progenitor cells, oligodendrocytes, astrocytes and microglia (Giera et al., 2018; Haitina et al., 2008), and GPR56/AGRG1 deficiency can cause severe neurodevelopmental diseases such as bilateral frontoparietal polymicrogyria (BFPP) (Sotnikova et al., 2004).

Without wishing to be bound by theory, dietary amino-acid availability can be important in the production of biogenic amines that can shape host physiology. The studies herein can highlight other members of the microbiota as an alternative source of substrates that are often thought of as primarily derived from diet (e.g., essential amino acids). Microbial-produced amino acids may potentially supplement or even replace dietary amino acids in microbial biotransformations. Microbe-derived L-Phe may be used as a substrate for biotransformation by M. morganii using a simplified and highly-artificial diet that lacks L-Phe. However, bacterial L-Phe may also be important under physiological conditions. For example, dietary amino acids are largely absorbed in the small intestine (Adibi, 1973); thus, colonic microbes such as M. morganii have relatively limited access to dietary amino acids as compared to small intestinal organisms. Also, low-protein diets naturally decrease microbial access to dietary amino acids, and fasting may reduce intestinal amino acid availability even further (Pezeshki et al., 2016). Thus, microbial production of amino acids in the colon may play a critical role in the production of various bioactive microbiota metabolites under a variety of physiologically relevant conditions.

Also provided are various therapeutic and diagnostic methods.

In one aspect is provided a method of preventing or treating monoamine oxidase inhibitor (MAOI)-induced toxicity in a subject. An antibiotic effective to target a bacterial strain comprising a nucleic acid sequence encoding a phenethylamine production gene is administered.

In a related aspect is provided a method of preventing or treating monoamine oxidase inhibitor (MAOI)-induced toxicity in a subject. A small molecule antagonist of a bacterial phenethylamine synthesis enzyme is administered.

In various embodiments, the method of preventing or treating MAOI-induced toxicity in a subject comprising administering an antibiotic effective to target Morganella spp.

Although M. morganii was previously reported to produce dopamine, the inventors found that all isolates of M. morganii primarily produced the potent trace amine phenethylamine rather than dopamine or tyramine. The inventors also found that treatment of M. morganii monocolonized mice with an MAOI led to systemic accumulation of phenethylamine and mortality. Phenethylamine is a potent neuroactive chemical that, unlike dopamine and tyramine, can readily cross the blood-brain barrier (Oldendorf, 1971). The effects of phenethylamine are thought to be mediated primarily through activation of the trace amine-associated receptors and subsequent release of norepinephrine and dopamine (Borowsky et al., 2001; Bunzow, 2001; Sotnikova et al., 2004). However, the data here suggests that phenethylamine also can act as a selective agonist for DRD2-4. It will be fascinating to dissect the cellular and molecular mechanisms by which microbiota-derived phenethylamine can influence host biology both locally and systemically.

Without wishing to be bound by theory, the inventors' findings suggest that interindividual variability in microbial production of phenethylamine may explain some of the variable effects of MAOIs on depression; this possibility is particularly intriguing given the reported beneficial effects of phenethylamine on mood and the ability of phenethylamine to cross the blood-brain barrier (Irsfeld et al., 2013). In addition, biogenic amine production by gut microbes may explain some of the side effects of MAOIs.

One of the most prominent adverse events associated with MAOIs is ‘tyramine poisoning’, which typically results from ingestion of food products containing high levels of tyramine, such as certain cheeses (Fiedorowicz, 2004). The data herein indicates that specific gut microbes also act as a source of biogenic amines that may have similar effects on host physiology and that it is possible that pharmacological inhibitors of biogenic amine receptors that are meant to act at specific sites (e.g., in the brain) may also impinge upon natural host-microbiota interactions.

Also provided is a method of treating a disease or condition caused by decreased MAO activity in a subject. An antibiotic effective to target a bacterial strain comprising a nucleic acid sequence encoding a phenethylamine production gene is administered.

In a related aspect is provided a method of treating a disease or condition caused by decreased MAO activity in a subject. A small molecule antagonist of a bacterial phenethylamine synthesis enzyme is administered.

The bacterial strain may produce phenethylamine, such as M. morganii. The bacterial strain may secrete phenethylamine. Various diseases or conditions caused by decreased MAO activity can be treated, including Brunner syndrome, autism or anti-social behavior. The subject may express a MAOA-L variant.

Various antibiotics effective to target Morganella morganii may be used, such as cefepime, piperacillin, tazobactam, ceftazidime, cefotaxime, ceftibuten, meropenem, doripenem, ertapenem, a fluoroquinolone, or an aminoglycoside.

Also provided is a method of treating depression in a subject comprising administering a bacterial strain comprising a nucleic acid sequence encoding a phenethylamine production gene. An anti-depressant, such as an MAOI, can also be administered to the subject.

Also provided is a method for evaluating potential toxicity of a monoamine oxidase inhibitor (MAOI) in a subject. A gastrointestinal microbiota sample is obtained from the subject. The sample is assayed for the presence of a bacterial strain comprising a phenethylamine production gene, such as Morganella morganii. The levels of the bacterial strain can also be quantitatively assayed so as to further assess the risk of potential MAOI toxicity. The presence of, and/or levels of, the bacterial strain can be used to assess the risk of MAOI toxicity. From this information, a different anti-depressant or therapeutic besides a MAOI may be administered. Alternatively, the dosage levels of MAOI may be adjusted or reduced.

Also provided is a method for evaluating method for evaluating potential efficacy of an MAOI in a subject. The method comprises taking a fecal sample or a sample from the gastrointestinal tract of the subject, and assaying for the presence of a bacterial strain (e.g., Morganella morganii) comprising a nucleic acid sequence encoding a phenethylamine production gene. A quantitative assay for the amount of the bacterial strain may also be conducted. The subject may then be treated with an MAOI. The dosage or type of MAOI can be adjusted based on the presence of, and/or amount of, the bacterial strain.

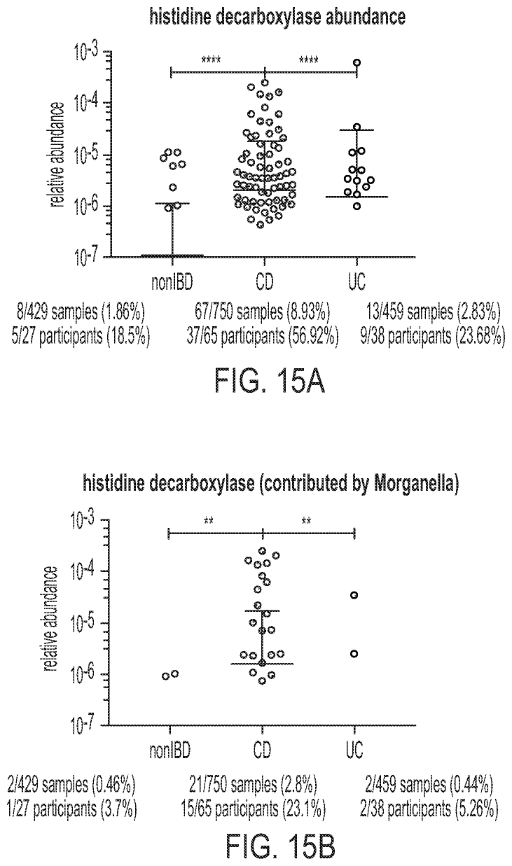

Also provided is a method for method of preventing or treating histamine-induced gastrointestinal disease in a subject. Notably, the inventors discovered that histidine decarboxylases, including M. morganii histidine decarboxylase, are enriched in patients with Crohn's disease as compared to healthy controls or UC patients (see FIGS. 15A and 15B), which may implicate histamine production by bacteria in CD. One or more antibiotics effective to target a bacterial strain comprising a nucleic acid sequence encoding a histamine production gene is administered to the subject. In a related aspect is provided preventing or treating histamine-induced gastrointestinal disease in a subject where a small molecule antagonist of histamine synthesis enzyme is administered. Exemplary small molecule antagonists of histamine synthesis enzymes include, but are not limited to, histidine decarboxylase inhibitors, such as rugosin D, rugosin A methyl ester, tellimagrandin II, rugosin A, pinocembrin, α-fluoromethylhistidine, brocresine, lecanoric acid, 2-hydroxy-5-carbomethoxybenzyloxyamine, and aminooxy analogs of histamine. Morganella histidine decarboxylase may be inhibited by rugosin D, rugosin A methyl ester, tellimagrandin II, and rugosin A. In a related aspect, general or specific histamine receptor antagonists are administered.

An exemplary disease is histamine intolerance, which can mimic the symptoms of food allergy. Another is diarrhea. Histamine-induced gastrointestinal diseases can be caused by, or exacerbated by, interference with the activity of the enzymes DAO and HNMT. Without wishing to be bound by theory, the presence of, or an excess amount of, L. reuteri or a bacterium of Morganella spp. can cause or exacerbate intestinal disease. These can directly convert L-His into histamine.

Another exemplary disease is inflammatory bowel disease. The abundance of Morganella spp. encoding histidine decarboxylase is increased in Crohn's disease versus healthy controls and ulcerative colitis (FIG. 15B). Without wishing to be bound by theory, the presence of, or an excess amount of Morganella spp. or histidine decarboxylase may cause or exacerbate Crohn's disease. Also provided is a method to treat patients that harbor Morganella spp. or other bacteria that encode histidine decarboxylase or an excess amount of bacteria that encode histidine decarboxylase. One or more antibiotics effective to target a bacterial strain comprising a nucleic acid sequence encoding a histamine production gene is administered. In a related aspect is provided preventing or treating histamine-induced gastrointestinal disease in a subject where a small molecule antagonist of histamine synthesis enzyme is administered. In a related aspect, general or specific histamine receptor antagonists are administered.

Also provided is a method of preventing or treating an allergy, or asthma, in a subject. One or more antibiotics effective to target a bacterial strain comprising a nucleic acid sequence encoding a histamine production gene is administered. The bacterial strain can be L. reuteri or a bacterium of Morganella spp. Without wishing to be bound by theory, the presence of either or both strains can increase overall histamine levels in the subject, which would induce or exacerbate allergies or asthma. Exemplary antibiotics include cefepime, piperacillin, tazobactam, ceftazidime, cefotaxime, ceftibuten, meropenem, doripenem, ertapenem, a fluoroquinolone, or an aminoglycoside.

The antibiotic may be selected for based on the outcome of a method for evaluating potential effectiveness of an antibiotic to treat a gastrointestinal condition or disease, allergy or asthma in a subject. The method comprises a) taking a fecal sample or a sample from the gastrointestinal tract of the subject, and b) assaying for the presence of a bacterial strain (e.g., bacterium of Morganella spp.) comprising a nucleic acid sequence encoding a histamine production gene. The method can comprise assaying for the amount of the bacterial strain present in the gastrointestinal tract. The subject may be treated with the antibiotic. In some embodiments, an adjusting or determining the dosage of the antibiotic is undertaken based on the presence and/or amount of the bacterial strain present.

Also provided is a method of preventing or treating a disease or condition resulting from production of phenethylamine, the method comprising administering one or more antibiotics effective to target a bacterial strain producing L-phenylalanine. In a related aspect is provided a method of preventing or treating a disease or condition resulting from production of phenethylamine, the method comprising administering a small molecule antagonist of a phenethylamine synthesis enzyme. The small molecule antagonist may be a Shikimate pathway inhibitor. The small molecule antagonist may be an antagonist of aromatic L-amino acid decarboxylase (AADC or AAAD), such as carbidopa, benserazide, methyldopa, 3′,4′,5,7-Tetrahydroxy-8-methoxyisoflavone (DFMD), 3-hydroxybenzylhydrazine, 3-amino-1-methyl-5H-pyrido[4,3-b]indole (Trp-P-2), and α-difluoromethyl DOPA.

Further provided is a method of preventing or treating phenylketonuria (PKU) in a subject, the method comprising administering one or more antibiotics effective to target a bacterial strain producing L-phenylalanine. Also provided is a related method of preventing or treating phenylketonuria (PKU) in a subject, the method comprising administering a small molecule targeting the bacterial phenylalanine synthesis enzymes of bacterial strains secreting L-phenylalanine for the treatment or prevention of diseases resulting from overproduction of L-phenylalanine.

Exemplary bacterial strains include B. thetaiotaomicron (e.g., strain C34). The method may comprise administration of an antibiotic effective to target the bacterial strain, such as ampicillin, clavulanate, tazobactam, a cephamycin, ticarcillin, piperacillin, a cephalosporin, a carbapenem, clindamycin, lincomycin, chloramphenicol, a nitroimidazole, a fluoroquinolone. Alternatively, a combination of antibiotics may be administered, such as (a) a combination of ampicillin and sulbactam, (b) a combination of ticarcillin and clavulanate, or (c) a combination of piperacillin and tazobactam.

In various embodiments of the above methods involving administration of an antibiotic, a probiotic composition may be administered. The probiotic composition may be administered after a course of antibiotics is administered, for example. Without wishing to be bound by theory, the probiotic composition may be effective to colonize the gut with different bacteria and prevent the targeted bacteria from quickly reestablishing itself. In additional embodiments, the antibiotic can be administered in a cyclic manner with the probiotic composition. The antibiotic could even be administered concurrently with the probiotic composition.

Also provided is a kit for evaluating potential toxicity of an MAOI in a subject. The kit can comprise a nucleic acid, antibody, or other reagent capable of binding specifically to a nucleotide or protein expressed by a bacterial strain, where the bacterial strain comprises a nucleic acid sequence encoding a phenethylamine production gene.

Also provided is a kit for evaluating potential effectiveness of an antibiotic to treat a gastrointestinal condition or disease, allergy or asthma in a subject. The kit comprises a nucleic acid, antibody, or other reagent capable of binding specifically to a nucleotide or protein expressed by a bacterial strain, where the bacterial strain comprises a nucleic acid sequence encoding a histamine production gene (such as one detected in Crohn's disease patients according to data shown in FIGS. 15A and 15B). The kit may be used to select for a patient who would benefit from treatment with a small molecule inhibitor of an enzymatic product of a histamine production gene (e.g., histamine decarboxylase inhibitor).

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

The following table shows the reagents used throughout the following examples:

| REAGENT or RESOURCE | SOURCE | IDENTIFIER |
|---|---|---|
| Bacterial Strains | | |
| B. fragilis | ATCC | 25285 |
| B. ovatus | ATCC | 8483 |
| B. thetaiotaomicron | ATCC | 29741 |
| B. uniformis | ATCC | 8492 |
| M. morganii | ATCC | 25830&49948 |
| Chemicals, peptides, and | | |
| Recombinant Proteins | | |
| Gifu Anaerobic Broth | VWR | 11007-214 |
| Bright-Glo ™Luciferase | Promega | E2620 |
| Assay System | | |
| Histamine Elisa Kit | Enzo Life Sciences | ENZ-KIT140-0001 |
| Phenethylamine | Sigma-Aldrich | 241008 |
| Tyramine | Sigma-Aldrich | T90344 |
| Dopamine | Sigma-Aldrich | H8502 |
| Histamine | Sigma-Aldrich | H7129 |
| Acetylcholine | Sigma-Aldrich | A2661 |
| L-DOPA | Sigma-Aldrich | D9628 |
| Succinate | Sigma-Aldrich | 398055 |
| Serotonin | Sigma-Aldrich | 14927 |
| Gastrin | Sigma-Aldrich | G9145 |
| Peptide YY | Anaspec | AS024401 |
| Pancreatic polypeptide | Anaspec | AS-22866 |
| Cholecystokinin | Sigma-Aldrich | C2175 |
| Trace mineral supplement | ATCC | MD-TMS |
| Vitamin supplement | ATCC | MD-VS |
| L-glycine | Sigma-Aldrich | G8898 |
| L-valine | Sigma-Aldrich | 94619 |
| L-leucine | Sigma-Aldrich | L8000 |
| L-isoleucine | Sigma-Aldrich | I2752 |
| L-methionine | Sigma-Aldrich | 64319 |
| L-histidine | Sigma-Aldrich | H8000 |
| L-arginine | Sigma-Aldrich | A5131 |
| L-phenylalanine | Sigma-Aldrich | P2126 |
| L-tyrosine | Sigma-Aldrich | T3754 |
| L-tryptophan | Sigma-Aldrich | T0254 |
| N-methylphenethylamine | Sigma-Aldrich | M68423 |
| Octopamine | Sigma-Aldrich | O0250 |
| Synephrine | Sigma-Aldrich | S0752 |
| Epinephrine | Sigma-Aldrich | E4250 |
| Norepinephrine | Sigma-Aldrich | A7257 |
| 3-Methoxytyramine | Sigma-Aldrich | M4251 |
| DMEM | Sigma-Aldrich | D6429 |
| RPMI 1640 | Thermo Fisher | 21870092 |

### Example 1. Testing of PRESTO-Salsa

To facilitate pooling of GPCR-barcode pairs, an Expi 293T cell line was generated that stably expresses the Barr-TEV fusion protein. These cells can be efficiently co-transfected with a plasmid that encodes a given GPCR-tTA fusion and a reporter plasmid that encodes a defined barcode downstream of a tet-responsive element. To enable concurrent screening of all GPCRs, the stable Expi 293T cell line was co-transfected with all 314 defined GPCR-barcode reporter plasmid pairs in individual wells in 96 well plates. After about 24 hours, all cells were mixed together in a single tube before the cellular mixtures were redistributed into approximately four 96 well plates. Importantly, Expi 293T cells are non-adherent, which facilitates pooling and redistribution of cells.

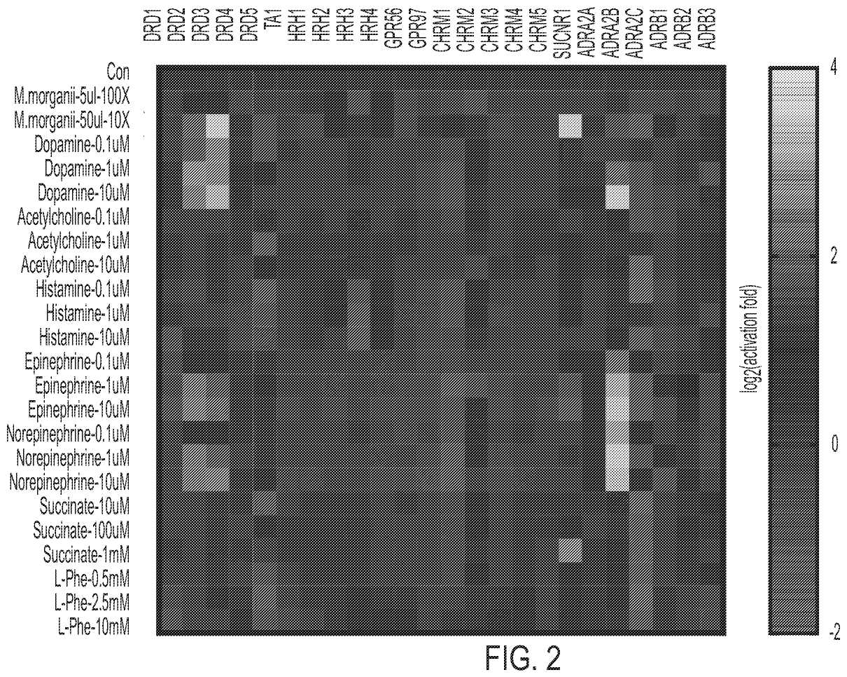

After redistribution, each well was stimulated with a given metabolite mixture before harvesting RNA, synthesizing cDNA and then sequencing barcodes via Illumina amplicon sequencing. Relative expression levels of a given barcode were compared to control samples so as to represent the relative activation of a given GPCR by that metabolite or metabolite mixture. To assess the sensitivity and specificity of PRESTO-Salsa, the inventors first established this system using well-known GPCR ligands or microbial metabolite mixtures that contain known GPCR agonists, as shown in FIG. 2.

These data demonstrate the specificity and sensitivity of the PRESTO-Salsa technology. Importantly, the sequencing-based readout used for PRESTO-Salsa was considerably more sensitive than PRESTO-Tango given the relative sensitivity of sequencing versus luciferase expression.

### Example 2. A Forward Chemical Genetic Screen Identifies Human Gut Microbes that Activate GPCRs

144 unique human gut bacteria spanning five phyla, nine classes, eleven orders, and twenty families were isolated from 11 inflammatory bowel disease patients via high-throughput anaerobic culturomics and massively barcoded 16S rRNA gene sequencing. All strains were cultured in gut microbiota medium (Goodman et al., 2011) or Gifu broth at 37° C. under anaerobic conditions and the identities of all strains were confirmed by 16S rRNA gene sequencing. The isolated human gut bacteria are listed in Table 1. Each isolate was grown in monoculture in a medium specialized for the cultivation of human gut microbes (gut microbiota medium; GMM).

Supernatants from individual bacterial monocultures were screened against the near-complete non-olfactory GPCRome (314 conventional GPCRs) using the high-throughput assay Parallel Receptor-ome Expression and Screening via Transcriptional Output-Tango (PRESTO-Tango).

The PRESTO-Tango assay was conducted as follows. HTLA cells, a HEK293 cell line that stably expresses β-arrestin-TEV and tTA-Luciferase (a kind gift from Gilad Barnea, Brown University), were plated in 96-well tissue culture plates (Eppendorf) in DMEM containing 10% FBS and 1% Penicillin/Streptomycin. One day after plating (after reaching approximately 90% confluence) Tango plasmids were transfected into HTLA cells using polyethylenimine (Polysciences). 16-24 hours after transfection, medium was replaced with 180 μl fresh DMEM containing 1% Penicillin/Streptomycin and 10 mM HEPES and 20 μl bacterial medium alone or bacterial supernatant. For PRESTO-Tango screening, all commensals were cultured in gut microbiota medium or Gifu broth for 2 days in an anaerobic chamber (Coy). Commensal supernatants were sterilized by high-speed centrifugation followed by sterile filtration (0.22 μm). For in vitro studies, M. morganii was cultured in minimal medium (MM), or MM with 10 mM L-Phe, 2.5 mM L-Tyr, 10 mM L-DOPA, or 10 mM L-His for 24 hours. Bacterial supernatants were analyzed by LC-MS.

16-24 hours after stimulation, 50 ul per well of Bright-Glo solution (Promega) diluted 20-fold with PBS containing 20 mM HEPES was added into each well. After 20 min incubation at room temperature, luminescence was quantified using a Spectramax i3x (Molecular Devices). Activation fold for each sample was calculated by dividing relative luminescence units (RLU) for each condition by RLUs from media alone controls.

GPCR activation by metabolomes from 144 bacterial strains isolated from the human gut microbiota as measured by PRESTO-Tango. Data is displayed as a heatmap (FIG. 3) or on a hierarchical tree of GPCRs organized by class, ligand type, and receptor family (FIG. 4). Shading intensity represents the maximum magnitude of activation (log 2) over background across the entire data set—i.e., the maximum activation of a given GPCR by any microbial metabolome in our collection. Radii of the circles at each tip are scaled based on the number of strains that activated a given receptor or receptor family (i.e., number of hits across the complete data set). Hits are defined as activation of a given receptor more than two-fold over background. Graphics were generated in collaboration with Visavisllc using d3.js.

TABLE 1

| No. | Family | Genus | Species |
|---|---|---|---|
| 1 | Bacteroidaceae | Bacteroides | fragilis |
| 2 | Enterobacteriaceae | Other | Other |
| 3 | Streptococcaceae | Streptococcus | luteciae |
| 4 | Clostridiaceae | Clostridium | perfringens |
| 5 | Fusobacteriaceae | Fusobacterium | spp. |
| 6 | Streptococcaceae | Streptococcus | spp. |
| 7 | Enterobacteriaceae | UC | UC |
| 8 | Enterobacteriaceae | UC | UC |
| 9 | Bacteroidaceae | Bacteroides | fragilis |
| 10 | Enterobacteriaceae | UC | UC |
| 11 | Veillonellaceae | Megasphaera | spp. |
| 12 | Clostridiaceae | Clostridium | perfringens |
| 13 | Bacteroidaceae | Bacteroides | fragilis |
| 14 | Bifidobacteriaceae | Bifidobacterium | UC |
| 15 | Bacteroidaceae | Bacteroides | fragilis |
| 16 | Enterobacteriaceae | | |
| 17 | Enterobacteriaceae | Morganella | spp. |
| 18 | Bacteroidaceae | Bacteroides | spp. |
| 19 | Bacteroidaceae | Bacteroides | ovatus |
| 20 | Bifidobacteriaceae | Bifidobacterium | UC |
| 21 | Bifidobacteriaceae | Bifidobacterium | UC |
| 22 | Bacteroidaceae | Bacteroides | fragilis |
| 23 | Enterobacteriaceae | UC | UC |
| 24 | Fusobacteriaceae | Fusobacterium | spp. |
| 25 | Actinobacteria | Bifidobacterium | adolescentis |
| 26 | Actinobacteria | Bifidobacterium | adolescentis |
| 27 | Enterobacteriaceae | UC | UC |
| 28 | Enterobacteriaceae | Morganella | spp. |
| 29 | Enterobacteriaceae | UC | UC |
| 30 | Alcaligenaceae | Sutterella | spp. |
| 31 | Enterobacteriaceae | UC | UC |
| 32 | Bacteroidaceae | Bacteroides | spp. |
| 33 | Enterobacteriaceae | UC | UC |
| 34 | Bacteroidaceae | Bacteroides | spp. |
| 35 | Bacteroidaceae | Bacteroides | fragilis |
| 36 | Actinobacteria | Bifidobacterium | adolescentis |
| 37 | Lactobacillaceae | Lactobacillus | UC |
| 38 | Lactobacillaceae | Pediococcus | UC |
| 39 | Lactobacillaceae | Pediococcus | UC |
| 40 | Enterobacteriaceae | UC | UC |
| 41 | Bacteroidaceae | Bacteroides | fragilis |
| 42 | Bifidobacteriaceae | Bifidobacterium | spp. |
| 43 | Bifidobacteriaceae | Bifidobacterium | UC |
| 44 | Enterobacteriaceae | Morganella | spp. |
| 45 | Streptococcaceae | Streptococcus | spp. |
| 46 | Erysipelotrichales | Erysipelotrichaceae | spp. |
| 47 | Erysipelotrichales | Erysipelotrichaceae | spp. |
| 48 | Clostridiaceae | Clostridium | perfringens |
| 49 | Bifidobacteriaceae | Bifidobacterium | UC |
| 50 | Erysipelotrichales | Erysipelotrichaceae | spp. |
| 51 | Coriobacteriaceae | Collinsella | aerofaciens |
| 52 | Enterobacteriaceae | UC | UC |
| 53 | Coriobacteriaceae | Collinsella | aerofaciens |
| 54 | Clostridiaceae | Clostridium | perfringens |
| 55 | Streptococcaceae | Streptococcus | spp. |
| 56 | Bacteroidaceae | Bacteroides | ovatus |
| 57 | Bacteroidaceae | Bacteroides | spp. |
| 58 | Bacteroidaceae | Bacteroides | fragilis |
| 59 | Bacteroidaceae | Bacteroides | UC |
| 60 | Enterobacteriaceae | UC | UC |
| 61 | Enterobacteriaceae | UC | UC |
| 62 | Bacteroidaceae | Bacteroides | fragilis |
| 63 | Enterobacteriaceae | UC | UC |
| 64 | Porphyromonadaceae | Parabacteroides | distasonis |
| 65 | Clostridiaceae | Clostridium | perfringens |
| 66 | Enterobacteriaceae | Morganella | spp. |
| 67 | Enterobacteriaceae | UC | UC |
| 68 | [Tissierellaceae] | Peptoniphilus | spp. |
| 69 | Enterobacteriaceae | Morganella | spp. |
| 70 | [Tissierellaceae] | Peptoniphilus | spp. |
| 71 | Enterobacteriaceae | UC | UC |
| 72 | Enterobacteriaceae | UC | UC |
| 73 | Coriobacteriaceae | Collinsella | aerofaciens |
| 74 | Lachnospiraceae | [Ruminococcus] | gnavus |
| 75 | Enterobacteriaceae | UC | UC |
| 76 | Lachnospiraceae | [Ruminococcus] | gnavus |
| 77 | Enterobacteriaceae | UC | UC |
| 78 | Enterobacteriaceae | UC | UC |
| 79 | Enterobacteriaceae | UC | UC |
| 80 | Bacteroidaceae | Bacteroides | spp. |
| 81 | Bacteroidaceae | Bacteroides | uniformis |
| 82 | Bacteroidaceae | Bacteroides | ovatus |
| 83 | Coriobacteriaceae | Collinsella | stercoris |
| 84 | Coriobacteriaceae | Collinsella | stercoris |
| 85 | Clostridiales (order) | UC | UC |
| 86 | Lachnospiraceae | UC | UC |
| 87 | Coriobacteriaceae | Collinsella | stercoris |
| 88 | Lactobacillaceae | Lactobacillus | reuteri |
| 89 | Lactobacillaceae | Lactobacillus | reuteri |
| 90 | Bacteroidaceae | Bacteroides | uniformis |
| 91 | Bacteroidaceae | Bacteroides | UC |
| 92 | Sinobacteriaceae | Nevskia | spp. |
| 93 | Lactobacillaceae | Lactobacillus | reuteri |
| 94 | Lactobacillaceae | Lactobacillus | reuteri |
| 95 | Sinobacteriaceae | Nevskia | spp. |
| 96 | Veillonellaceae | Acidaminococcus | spp. |
| 97 | Bacteroidaceae | Bacteroides | uniformis |
| 98 | Bacteroidaceae | Bacteroides | uniformis |
| 99 | Bifidobacteriaceae | Bifidobacterium | UC |
| 100 | Veillonellaceae | Acidaminococcus | spp. |
| 101 | Veillonellaceae | Acidaminococcus | spp. |
| 102 | Bacteroidaceae | Bacteroides | UC |
| 103 | Veillonellaceae | Acidaminococcus | spp. |
| 104 | Veillonellaceae | Acidaminococcus | spp. |
| 105 | Bifidobacteriaceae | Bifidobacterium | UC |
| 106 | Bifidobacteriaceae | Bifidobacterium | UC |
| 107 | Bifidobacteriaceae | Bifidobacterium | UC |
| 108 | Bifidobacteriaceae | Bifidobacterium | UC |
| 109 | Bacteroidaceae | Bacteroides | uniformis |
| 110 | Bacteroidaceae | Bacteroides | ovatus |
| 111 | Veillonellaceae | Megasphaera | spp. |
| 112 | Lachnospiraceae | [Ruminococcus] | gnavus |
| 113 | Lactobacillaceae | Pediococcus | UC |
| 114 | Lactobacillaceae | Pediococcus | UC |
| 115 | Porphyromonadaceae | Parabacteroides | distasonis |
| 116 | Coriobacteriaceae | Eggerthela | lenta |
| 117 | Lachnospiraceae | Blautia | producta |
| 118 | Enterobacteriaceae | UC | UC |
| 119 | Ruminococcaceae | Faecalibacterium | prausnitzii |
| 120 | Clostridiaceae | Clostridium | perfringens |
| 121 | Streptococcaceae | Streptococcus | spp. |
| 122 | Coriobacteriaceae | Eggerthela | lenta |
| 123 | Enterobacteriaceae | UC | UC |
| 124 | Enterobacteriaceae | UC | UC |
| 125 | Not yet identified | | |
| 126 | Lachnospiraceae | [Ruminococcus] | torques |
| 127 | Bacteroidaceae | Bacteroides | uniformis |
| 128 | Erysipelotrichaceae | Allobaculum | spp. |
| 129 | Porphyromonadaceae | Parabacteroides | spp. |
| 130 | Coriobacteriaceae | Collinsella | stercoris |
| 131 | Not yet identified | | |
| 132 | [Tissierellaceae] | Peptoniphilus | spp. |
| 133 | Bacteroidaceae | Bacteroides | fragilis |
| 134 | Ruminococcaceae | Oscillospira | spp. |
| 135 | Enterobacteriaceae | Morganella | spp. |
| 136 | Enterobacteriaceae | UC | UC |
| 137 | Coriobacteriaceae | Eggerthela | lenta |
| 138 | Enterobacteriaceae | Morganella | spp. |
| 139 | Ruminococcaceae | Oscillospira | spp. |
| 140 | Ruminococcaceae | Oscillospira | spp. |
| 141 | Mogibacteriaceae | Mogibacterium | spp. |
| 142 | Moraxellaceae | Enhydrobacter | spp. |
| 143 | Not yet identified | Not yet identified | |
| 144 | Enterobacteriaceae | UC | UC |

The culture collection can allow for examination of the effects of phylogenetically diverse taxa while also providing insights into potential strain-specific differences between members of the same species.

An analysis of the above cultures indicated that human gut microbes produce compounds that activate both well-characterized and orphan GPCRs. PRESTO-Tango screening revealed a diverse array of hits, including bacterial-derived metabolite mixtures that activated well-characterized GPCRs as well as mixtures that activated orphan receptors. According to the data in FIG. 4, activation of at least one GPCR from every class by at least one metabolite mixture was observed across the complete data set.

Specific patterns of GPCR activation emerged based on gross phylogeny. Tables 2 and 3 show the degree of receptor activation for each bacteria according to PRESTO-Tango analysis in each of GMM and Gifu media, respectively. For example, most strains belonging to the phyla Bacteroidetes and Proteobacteria potently activated the succinate receptor (Sucr1), while strains belonging to the phyla Firmicutes, Fusobacteria and Actinobacteria largely failed to activate this receptor (Tables 2-3). However, many activation patterns did not correlate with phylogeny, including multiple examples of bacterial strains that exhibited unique GPCR agonist activities despite being assigned to the same species (Tables 2-3).

TABLE 2
PRESTO-Tango with GMM

| | Gut | Erysipelo- | Erysipelo- | Erysipelo- | | | | |
|---|---|---|---|---|---|---|---|---|
| PRESTO-Tango | Microbiota | trichaceae | trichaceae | trichaceae | Ruminococcus | Ruminococcus | Ruminococcus | Ruminococcus |
| with GMM | Medium | spp-1# | spp-2# | spp-3# | gnavus-1# | gnavus-2# | gnavus-3# | torques |
| ADCYAP1R1- | 0 | 0.133 | −0.060 | −0.281 | 0.720 | 0.158 | −0.167 | −0.511 |
| Tango | | | | | | | | |
| ADORA1-Tango | 0 | 0.189 | 0.023 | −0.504 | 0.944 | 0.234 | −0.262 | −1.023 |
| ADORA2A-Tango | 0 | 0.237 | 0.244 | −0.038 | 0.195 | −0.018 | −1.027 | −0.672 |
| ADORA2B-Tango | 0 | 0.067 | 0.144 | −0.209 | −0.417 | −0.329 | −0.816 | −0.293 |
| ADORA3-Tango | 0 | 0.567 | 0.503 | 0.294 | 0.165 | 0.092 | −0.157 | −0.319 |
| ADRA1A-Tango | 0 | 0.249 | −0.041 | −0.199 | 0.987 | 0.021 | −0.608 | −0.603 |
| ADRA1B-Tango | 0 | 0.020 | −0.258 | −0.225 | 0.930 | −0.279 | −0.229 | −1.010 |
| ADRA1D-Tango | 0 | −0.197 | −0.169 | −0.381 | 1.116 | 0.260 | −0.164 | −1.274 |
| ADRA2A-Tango | 0 | 0.132 | −0.346 | 0.261 | 1.525 | 0.220 | −0.395 | −0.585 |
| ADRA2B-Tango | 0 | −0.391 | −1.677 | −0.475 | 1.484 | 0.793 | −1.128 | −1.455 |
| ADRA2C-Tango | 0 | 0.216 | 0.122 | −0.345 | 1.862 | 0.397 | −1.163 | −0.955 |
| ADRB1-Tango | 0 | 0.049 | 0.025 | −0.033 | 1.183 | 0.705 | 0.040 | −0.463 |
| ADRB2-Tango | 0 | −0.551 | −0.249 | 0.426 | 1.103 | 0.959 | −0.319 | −0.200 |
| ADRB3-Tango | 0 | 0.000 | −0.136 | 0.415 | −0.095 | 0.118 | −0.524 | 0.245 |
| AGTR1-Tango | 0 | 0.216 | 0.290 | 0.000 | 0.747 | 0.504 | −0.360 | −0.042 |
| AGTR2-Tango | 0 | 0.038 | 0.031 | 0.207 | 0.974 | 0.996 | −0.026 | −0.618 |
| APJ-Tango | 0 | −0.326 | 0.110 | −0.535 | 0.730 | 0.326 | −0.526 | −1.093 |
| AVPR1A-Tango | 0 | 0.000 | −0.494 | 0.126 | 0.354 | 0.253 | −0.695 | −0.322 |
| AVPR1B-Tango | 0 | −0.306 | 0.133 | −0.155 | 0.491 | 0.031 | −0.122 | −0.754 |
| AVPR2-Tango | 0 | −0.065 | −0.003 | −0.259 | 0.551 | 0.115 | −0.207 | −0.078 |
| BB3-Tango | 0 | 0.132 | 0.188 | −0.321 | 0.407 | −0.157 | −0.386 | −0.792 |
| BDKBR1-Tango | 0 | 0.032 | −0.105 | −0.096 | 0.199 | −0.037 | −0.344 | −0.003 |
| BDKBR2-Tango | 0 | 0.321 | 0.349 | 0.259 | 0.662 | 0.669 | 0.408 | −0.041 |
| C3AR1-Tango | 0 | −0.081 | −0.279 | 0.087 | 0.580 | −0.163 | 0.239 | −0.190 |
| C5A-Tango | 0 | 0.327 | 0.447 | 0.481 | −0.062 | 0.634 | −0.159 | −0.787 |
| CALCRb-Tango | 0 | −0.175 | −0.273 | 0.095 | −0.160 | −0.320 | −0.695 | −0.107 |
| CALCRL-Tango | 0 | −0.287 | −0.024 | −0.304 | 0.163 | −0.309 | −0.554 | 0.027 |
| CASR-Tango | 0 | 0.053 | 0.085 | −0.367 | −0.222 | −0.487 | −0.643 | −0.846 |
| CCKAR-Tango | 0 | 0.182 | 0.103 | 0.167 | 0.233 | 0.192 | −0.061 | −0.218 |
| CCKBR-Tango | 0 | 0.018 | 0.067 | −0.268 | 1.071 | 0.119 | −0.271 | −0.241 |
| CCR10-Tango | 0 | −0.382 | −0.588 | 0.000 | 0.282 | 0.131 | −0.596 | −0.655 |
| CCR2-Tango | 0 | −0.119 | −0.156 | 0.237 | 0.234 | 0.292 | −0.292 | −0.779 |
| CCR3-Tango | 0 | 0.561 | 0.393 | 0.057 | 0.831 | 0.975 | −0.976 | −0.780 |
| CCR4-Tango | 0 | 0.265 | 0.307 | 0.354 | 1.058 | 0.610 | −0.424 | −1.056 |
| CCR5-Tango | 0 | 0.070 | 0.028 | 0.507 | 0.384 | 0.236 | −0.572 | −0.587 |
| CCR6-Tango | 0 | 0.145 | −0.183 | 0.492 | 1.732 | 1.158 | −0.410 | −0.652 |
| CCR7-Tango | 0 | 0.309 | 0.404 | 0.428 | 1.077 | 0.255 | −0.292 | −0.919 |
| CCR8-Tango | 0 | −0.334 | −0.334 | −0.680 | 0.891 | 0.304 | −0.541 | −1.416 |
| CCRL2-Tango | 0 | 0.590 | 0.193 | 0.679 | 0.312 | −0.257 | −0.563 | −0.497 |
| CD97-Tango | 0 | 0.170 | 0.170 | −0.606 | −0.192 | −0.157 | −0.235 | −0.261 |
| CHRM1-Tango | 0 | −0.585 | −0.260 | −0.418 | 0.588 | 0.058 | −0.712 | 0.000 |
| CHRM2-Tango | 0 | 0.046 | 0.002 | −0.688 | 0.791 | −0.620 | −0.157 | 0.371 |
| CHRM3-Tango | 0 | −0.196 | −0.140 | −0.628 | 1.554 | 0.232 | −0.310 | 0.138 |
| CHRM4-Tango | 0 | −0.209 | −0.453 | 0.214 | 0.153 | 0.189 | −0.790 | 0.085 |
| CHRM5-Tango | 0 | 0.340 | 0.277 | 0.429 | 0.371 | 0.428 | −0.349 | −0.283 |
| CMKLR1-Tango | 0 | −0.008 | 0.015 | 0.208 | 0.084 | 0.436 | −0.742 | −0.176 |
| CNR1-Tango | 0 | −0.095 | 0.110 | −0.516 | 0.436 | −0.388 | −0.411 | −0.751 |
| CNR2-Tango | 0 | −0.160 | −0.217 | 0.031 | 0.391 | −0.045 | −0.812 | −0.387 |
| CRHR1-Tango | 0 | 0.396 | 0.225 | −0.293 | 0.260 | 0.260 | −0.888 | −0.585 |
| CRHR2-Tango | 0 | 0.057 | 0.062 | −0.288 | 0.000 | 0.129 | 0.189 | −0.334 |
| CX3C1-Tango | 0 | −0.018 | 0.272 | −0.570 | 1.107 | 0.000 | 0.091 | −0.313 |
| CXCR1-Tango | 0 | 0.165 | 0.144 | 0.378 | −0.015 | −0.079 | −0.611 | −0.365 |
| CXCR2-Tango | 0 | 0.311 | −0.079 | 0.238 | 0.702 | 1.533 | −0.588 | −0.722 |
| CXCR3-Tango | 0 | 0.277 | 0.476 | −0.036 | 0.722 | 0.936 | −0.344 | 0.033 |
| CXCR4-Tango | 0 | 0.703 | 0.705 | 0.455 | 0.629 | 0.697 | −0.345 | −0.175 |
| CXCR5-Tango | 0 | 0.150 | 0.534 | 0.043 | 0.724 | 0.115 | −0.628 | −0.121 |
| CXCR6-Tango | 0 | −0.335 | −0.798 | −0.105 | 0.216 | 0.181 | 0.037 | −0.186 |
| CXCR7-Tango | 0 | −0.388 | −0.173 | −0.559 | 1.395 | 0.343 | −0.655 | −1.188 |
| CYSLTR1-Tango | 0 | 0.036 | 0.272 | 0.299 | 0.950 | 1.176 | −0.389 | −1.348 |
| CYSLTR2-Tango | 0 | 0.239 | 0.081 | 0.776 | 0.367 | 0.149 | −1.082 | −0.922 |
| DRD1-Tango | 0 | 0.151 | −0.116 | 0.155 | 0.555 | 0.134 | −0.710 | −0.727 |
| DRD2-Tango | 0 | 0.329 | 0.210 | −0.360 | 0.043 | −0.544 | −0.735 | −1.792 |
| DRD3-Tango | 0 | 0.991 | 0.193 | 0.082 | 0.088 | −0.373 | −2.273 | −1.708 |
| DRD4-Tango | 0 | 1.258 | 0.685 | 0.404 | 0.101 | 0.000 | −2.174 | −2.296 |
| DRD5-Tango | 0 | −0.220 | −0.150 | −0.053 | 0.216 | −0.078 | −0.616 | −0.372 |
| EDNRA-Tango | 0 | 0.244 | 0.226 | 0.233 | 0.489 | 0.137 | −0.044 | −0.066 |
| EDNRB-Tango | 0 | 0.230 | 0.268 | −0.106 | 0.270 | 0.145 | −0.598 | −0.122 |
| ELTD1-Tango | 0 | 0.280 | 0.243 | −0.374 | 0.543 | 0.051 | −0.799 | −0.957 |
| F2R-Tango | 0 | 0.015 | 0.100 | 0.014 | 0.511 | 0.189 | −0.090 | −0.141 |
| F2RL1-Tango | 0 | 0.002 | −0.018 | −0.055 | 0.310 | 0.163 | −0.204 | −0.314 |
| F2RL2-Tango | 0 | 0.381 | 0.337 | 0.326 | 0.003 | −0.284 | −0.284 | −0.389 |
| F2RL3-Tango | 0 | −0.025 | −0.063 | −0.003 | 0.204 | −0.178 | −0.345 | −0.057 |
| FFA1-Tango | 0 | 0.502 | 0.430 | 0.115 | 0.832 | 0.550 | 0.079 | −0.199 |
| FFA2-Tango | 0 | 0.631 | 0.433 | −0.083 | 1.061 | 0.576 | −0.649 | −0.614 |
| FFA3-Tango | 0 | 0.345 | 0.372 | 0.345 | 0.736 | 0.245 | −0.141 | −0.406 |
| FPR1-Tango | 0 | 1.373 | 1.299 | 1.166 | 0.678 | 0.356 | −0.426 | 0.386 |
| FPR2-Tango | 0 | 0.800 | 0.607 | 0.371 | 2.058 | −0.012 | −0.501 | −0.316 |
| FPR3-Tango | 0 | −0.029 | −0.029 | 0.038 | 0.677 | 0.418 | −0.593 | −0.293 |
| FSHR-Tango | 0 | 0.278 | 0.148 | −0.215 | −0.084 | −0.393 | −0.348 | −0.464 |
| GABBR1-Tango | 0 | −0.104 | −0.104 | −0.091 | 2.454 | −0.163 | −0.796 | −0.313 |
| GAL1-Tango | 0 | −0.184 | 0.000 | −0.526 | 1.319 | −0.357 | −0.771 | −1.186 |
| GAL2-Tango | 0 | 0.165 | −0.298 | 0.054 | −0.335 | −0.528 | −0.458 | −0.071 |
| GAL3-Tango | 0 | −0.465 | −0.531 | −0.698 | 0.405 | 0.100 | −0.638 | −1.390 |
| GCGR-Tango | 0 | 0.115 | 0.358 | −0.136 | 0.459 | 0.112 | −0.645 | 0.140 |
| GHRHR-Tango | 0 | 0.123 | 0.070 | 0.173 | 0.063 | 0.047 | −0.160 | 0.343 |
| GHSR-Tango | 0 | 0.273 | 0.261 | 0.122 | 0.756 | 0.580 | −0.411 | −0.103 |
| GIPR-Tango | 0 | −0.465 | −0.297 | 0.000 | 0.178 | 0.139 | −0.691 | −0.122 |
| GLP1R-Tango | 0 | 0.023 | 0.064 | −0.351 | −0.293 | −0.714 | −0.442 | −0.389 |
| GLP2R-Tango | 0 | −0.226 | −0.079 | −0.313 | 0.208 | 0.816 | 0.103 | −0.418 |
| GNRHR-Tango | 0 | −0.087 | −0.580 | −0.075 | −1.062 | −0.537 | −0.312 | −0.191 |
| GPBA-Tango | 0 | −0.954 | −0.248 | −0.246 | 0.877 | 0.846 | −0.993 | −0.559 |
| GPER-Tango | 0 | −0.100 | 0.131 | 0.206 | 0.679 | 0.180 | −0.890 | −0.408 |
| GPR1-Tango | 0 | 0.141 | 0.191 | −0.166 | 0.022 | 0.014 | −0.408 | −0.511 |
| GPR101-Tango | 0 | 0.166 | −0.029 | 0.321 | 0.322 | 0.392 | 0.127 | −0.319 |
| GPR110-Tango | 0 | −0.161 | −0.046 | −0.694 | 0.274 | −0.655 | −0.325 | −0.895 |
| GPR111-Tango | 0 | −0.515 | −0.441 | −0.324 | −0.194 | −0.523 | −0.423 | −0.245 |
| GPR113-Tango | 0 | −0.180 | −0.068 | −0.331 | 0.008 | 0.000 | 0.133 | −0.605 |
| GPR114-Tango | 0 | −0.200 | 0.005 | −0.211 | −0.155 | −0.013 | −0.415 | −0.143 |
| GPR115-Tango | 0 | 0.220 | 0.220 | −0.505 | 0.094 | −0.197 | 0.168 | −0.640 |
| GPR116-Tango | 0 | −0.027 | −0.125 | −0.594 | 0.085 | −0.039 | −0.400 | −0.639 |
| GPR119-Tango | 0 | −0.107 | 0.000 | −0.428 | −0.096 | 0.202 | −0.538 | −0.538 |
| GPR12-Tango | 0 | −0.039 | −0.045 | −0.265 | 0.871 | 0.608 | −0.106 | −0.009 |
| GPR120-Tango | 0 | 0.160 | −0.198 | −0.477 | 1.235 | 0.360 | −0.825 | −0.655 |
| GPR123-Tango | 0 | 0.210 | 0.264 | 0.366 | 0.645 | 0.371 | −0.220 | −0.296 |
| GPR124-Tango | 0 | 0.337 | 0.234 | −0.114 | 0.005 | 0.262 | −0.543 | −0.681 |
| GPR125-Tango | 0 | −0.085 | −0.004 | −0.056 | 0.069 | 0.147 | −0.572 | −1.185 |
| GPR126-Tango | 0 | −0.013 | −0.166 | −0.697 | 0.601 | −0.148 | −0.538 | −0.339 |
| GPR132-Tango | 0 | −0.239 | −0.084 | −0.476 | 0.572 | −0.535 | −0.418 | −0.679 |
| GPR133-Tango | 0 | 0.026 | 0.132 | −0.433 | 0.358 | 0.108 | −0.141 | −0.504 |
| GPR135-Tango | 0 | −0.286 | −0.448 | 0.181 | −0.012 | −0.113 | −0.252 | 0.009 |
| GPR141-Tango | 0 | 0.075 | 0.147 | −0.147 | 1.241 | 0.288 | −0.687 | −0.806 |
| GPR142-Tango | 0 | −0.531 | −0.155 | −0.119 | 0.617 | 0.320 | −0.314 | −0.769 |
| GPR143-Tango | 0 | 0.383 | −0.225 | −0.190 | 0.464 | −0.341 | −0.553 | −0.897 |
| GPR144-Tango | 0 | 0.057 | −0.298 | −0.341 | −0.695 | −0.371 | −0.534 | −0.534 |
| GPR146-Tango | 0 | 0.866 | 0.718 | 0.170 | −0.931 | −0.335 | −0.462 | −0.632 |
| GPR148-Tango | 0 | 0.093 | −0.170 | −0.122 | −0.160 | −0.879 | −0.940 | −0.658 |
| GPR149-Tango | 0 | 0.515 | 0.302 | 0.095 | 0.149 | 0.046 | −0.354 | −0.855 |
| GPR15-Tango | 0 | 0.335 | 0.335 | 0.077 | 1.019 | 0.197 | −0.824 | −0.409 |
| GPR150-Tango | 0 | 0.000 | 0.000 | 0.000 | 1.127 | 0.692 | −0.159 | 0.322 |
| GPR151-Tango | 0 | 0.327 | 0.197 | −0.377 | 0.534 | 0.357 | −0.086 | −0.067 |
| GPR152-Tango | 0 | −0.071 | −0.147 | −0.488 | 0.382 | 0.173 | −0.072 | −0.455 |
| GPR153-Tango | 0 | 0.364 | 0.248 | 0.204 | 0.040 | 0.254 | −0.167 | −0.318 |
| GPR156-Tango | 0 | −0.556 | −0.556 | −0.516 | 0.916 | 0.158 | −0.078 | −0.519 |
| GPR157-Tango | 0 | 0.111 | 0.141 | 0.299 | 0.282 | 0.108 | −0.538 | −0.571 |
| GPR158-Tango | 0 | −0.162 | −0.260 | −0.898 | 0.288 | 0.132 | −0.120 | −0.470 |
| GPR160-Tango | 0 | 0.367 | 0.298 | −0.139 | 0.514 | 0.106 | −0.149 | −0.451 |
| GPR161-Tango | 0 | 0.323 | 0.362 | 0.352 | 0.365 | 0.155 | −0.672 | −0.784 |
| GPR162-Tango | 0 | −0.045 | 0.116 | −0.048 | 0.004 | −0.142 | −0.919 | −0.361 |
| GPR17-Tango | 0 | 0.203 | 0.373 | −0.091 | 0.687 | 0.747 | −0.618 | −1.035 |
| GPR171-Tango | 0 | 0.297 | 0.179 | 0.085 | 0.618 | 0.246 | −0.722 | −0.788 |
| GPR173-Tango | 0 | −0.400 | −0.140 | 0.343 | 0.853 | 0.133 | −0.716 | −0.583 |
| GPR174-Tango | 0 | 0.906 | 0.410 | −0.093 | 0.378 | 0.509 | −0.675 | −0.410 |
| GPR18-Tango | 0 | 0.171 | 0.374 | −0.052 | 0.287 | 0.182 | −0.808 | −0.612 |
| GPR182-Tango | 0 | 0.514 | 0.610 | 0.327 | 0.241 | 0.396 | −0.570 | −0.804 |
| GPR183-Tango | 0 | 0.548 | 0.296 | −0.011 | 0.134 | 0.311 | −0.910 | −0.176 |
| GPR19-Tango | 0 | 0.386 | 0.467 | 0.079 | 0.228 | 0.201 | −0.409 | −0.908 |
| GPR20-Tango | 0 | 1.372 | 1.000 | 1.087 | 0.000 | 0.243 | −0.095 | −0.095 |
| GPR21-Tango | 0 | −0.206 | 0.229 | −0.202 | 0.133 | 0.155 | −0.327 | −0.739 |
| GPR22-Tango | 0 | 0.296 | 0.072 | −0.175 | 0.423 | 0.157 | −0.467 | −0.911 |
| GPR25-Tango | 0 | 0.144 | 0.207 | −0.045 | 0.040 | 0.063 | −0.404 | −0.426 |
| GPR26-Tango | 0 | 0.229 | −0.012 | 0.270 | 1.000 | 0.639 | 0.709 | −0.634 |
| GPR27-Tango | 0 | 0.440 | −0.020 | −0.242 | −0.068 | 0.240 | −0.657 | −0.168 |
| GPR3-Tango | 0 | 0.484 | 0.365 | −0.250 | 0.797 | 0.604 | 0.079 | −0.246 |
| GPR31-Tango | 0 | 0.564 | 0.717 | 0.033 | 0.826 | 0.392 | 0.031 | −0.454 |
| GPR32-Tango | 0 | −0.171 | −0.171 | −0.072 | 0.733 | 0.693 | −0.289 | −0.264 |
| GPR34-Tango | 0 | 0.167 | 0.233 | −0.296 | 0.644 | −0.215 | −0.286 | −0.129 |
| GPR35-Tango | 0 | 0.126 | 0.272 | −0.053 | 0.382 | 0.141 | −0.177 | −0.198 |
| GPR37-Tango | 0 | 0.379 | 0.377 | 0.285 | 0.122 | −0.501 | 0.041 | −0.428 |
| GPR37L1-Tango | 0 | 0.048 | 0.122 | 0.035 | 0.043 | −0.145 | −0.211 | −0.495 |
| GPR39-Tango | 0 | −0.200 | −0.320 | −0.269 | 0.424 | −0.046 | −0.136 | −0.452 |
| GPR4-Tango | 0 | 0.344 | 0.393 | 0.036 | −0.167 | −0.433 | 0.265 | −0.122 |
| GPR44-Tango | 0 | −0.221 | −0.148 | 0.000 | 0.033 | −0.652 | −0.388 | −0.027 |
| GPR45-Tango | 0 | 0.000 | −0.144 | −0.506 | 0.665 | 0.900 | −0.534 | −0.299 |
| GPR50-Tango | 0 | 0.345 | 0.221 | 0.544 | 0.525 | 0.259 | −0.104 | −0.361 |
| GPR52-Tango | 0 | −0.189 | −0.274 | 0.051 | 1.098 | 0.124 | 0.010 | −0.383 |
| GPR55-Tango | 0 | 0.554 | 0.079 | −0.081 | 0.549 | −0.196 | −0.241 | 0.016 |
| GPR56-Tango | 0 | 0.442 | 0.344 | −0.333 | 0.736 | −0.294 | −0.345 | 0.081 |
| GPR6-Tango | 0 | 0.528 | 0.402 | 0.220 | 0.651 | 0.367 | −0.551 | −0.093 |
| GPR61-Tango | 0 | 0.178 | −0.011 | 0.253 | 0.230 | 0.044 | 0.194 | 0.047 |
| GPR62-Tango | 0 | 0.264 | 0.000 | 0.140 | 0.350 | 0.030 | −0.162 | 0.194 |
| GPR63-Tango | 0 | −0.362 | −0.148 | −0.153 | 0.845 | 0.239 | −0.493 | −0.814 |
| GPR64-Tango | 0 | −0.261 | −0.414 | −0.287 | 0.407 | 0.020 | −0.251 | −0.282 |
| GPR65-Tango | 0 | −0.035 | −0.044 | −0.068 | 0.246 | 0.324 | −0.092 | −0.302 |
| GPR68-Tango | 0 | −0.097 | −0.004 | −0.256 | −0.045 | −0.379 | −0.274 | −0.379 |
| GPR75-Tango | 0 | 0.024 | −0.114 | −0.073 | 0.637 | 0.628 | −0.341 | −0.585 |
| GPR77-Tango | 0 | −0.354 | −0.506 | −0.010 | 0.524 | 0.351 | −0.257 | −0.459 |
| GPR78-Tango | 0 | 0.373 | 0.195 | 0.262 | 0.534 | 0.449 | −0.011 | −0.288 |
| GPR82-Tango | 0 | 0.187 | 0.078 | −0.043 | 0.413 | 0.706 | −0.046 | −0.529 |
| GPR83-Tango | 0 | 0.118 | 0.186 | −0.031 | 1.101 | 0.147 | −0.145 | −0.494 |
| GPR84-Tango | 0 | 0.350 | 0.367 | 0.172 | 0.798 | 0.636 | −0.265 | −0.825 |
| GPR85-Tango | 0 | 0.357 | 0.206 | 0.016 | 0.651 | 0.500 | −0.450 | −0.455 |
| GPR87-Tango | 0 | 0.388 | 0.372 | 0.357 | 0.546 | 1.022 | −0.168 | −0.565 |
| GPR88-Tango | 0 | −0.150 | −0.039 | 0.077 | 0.492 | 0.138 | −0.388 | −0.245 |
| GPR97-Tango | 0 | 0.134 | −0.308 | −0.118 | 0.822 | 0.436 | −0.411 | −0.832 |
| GPRC5A-Tango | 0 | 0.501 | 0.429 | 0.285 | 0.814 | 0.603 | −0.311 | −0.552 |
| GPRC5B-Tango | 0 | 0.397 | 0.293 | 0.082 | 0.417 | 0.389 | −0.338 | −0.918 |
| GPRC5C-Tango | 0 | 0.475 | 0.504 | 0.298 | 0.976 | 0.351 | −0.643 | −0.949 |
| GPRC5D-Tango | 0 | −0.278 | −0.364 | −0.223 | 1.037 | 0.173 | −0.314 | −0.545 |
| GPRC6A-Tango | 0 | −0.272 | −0.397 | −0.805 | −0.054 | −0.486 | −0.137 | −0.713 |
| GRM1-Tango | 0 | −0.016 | −0.191 | −0.457 | 0.374 | −0.012 | −0.466 | −0.615 |
| GRM2-Tango | 0 | −0.134 | −0.125 | −0.628 | 0.769 | −0.749 | −0.244 | −0.807 |
| GRM4-Tango | 0 | 0.276 | 0.091 | 0.128 | 0.280 | −0.759 | −0.147 | −0.818 |
| GRM5-Tango | 0 | −0.558 | −0.484 | −0.564 | −0.047 | −0.449 | −0.056 | −0.386 |
| GRM6-Tango | 0 | −0.545 | −0.717 | −0.157 | −0.045 | −0.230 | −0.531 | 0.098 |
| GRM7-Tango | 0 | −0.194 | −0.443 | −1.066 | −0.409 | −0.302 | −0.268 | −0.945 |
| GRM8-Tango | 0 | −0.434 | −0.406 | −0.435 | 0.800 | −0.806 | −0.631 | −0.689 |
| GRPR-Tango | 0 | 0.145 | 0.016 | 0.028 | 0.417 | −0.260 | −0.345 | −0.620 |
| HCA1-Tango | 0 | 0.409 | 0.892 | 0.357 | 0.846 | 0.569 | −0.871 | −0.494 |
| HCA2-Tango | 0 | 0.485 | 0.385 | 0.396 | 0.851 | 0.941 | −0.652 | −1.250 |
| HCA3-Tango | 0 | 0.597 | 0.977 | −0.706 | 0.751 | 0.666 | −0.874 | −0.738 |
| HCTR1-Tango | 0 | 0.600 | 0.760 | −0.160 | 0.867 | 1.270 | −0.407 | −0.623 |
| HCTR2-Tango | 0 | −0.160 | 0.317 | 0.112 | 0.511 | 0.766 | 0.000 | −0.778 |
| HRH1-Tango | 0 | 0.258 | 0.336 | 0.266 | 0.115 | 0.841 | −0.874 | −1.459 |
| HRH2-Tango | 0 | 0.235 | 0.609 | 0.108 | 0.185 | 0.408 | −0.906 | −0.391 |
| HRH3-Tango | 0 | −0.372 | −0.481 | −0.366 | 0.212 | 1.205 | −1.464 | −1.324 |
| HRH4-Tango | 0 | 0.081 | 0.224 | −0.111 | 0.325 | 1.156 | −0.658 | −0.949 |
| HTR1A-Tango | 0 | 0.772 | 0.777 | 0.250 | 1.765 | 0.370 | −0.597 | −0.295 |
| HTR1B-Tango | 0 | 0.647 | 0.697 | 0.301 | 0.728 | −0.005 | −0.359 | −0.824 |
| HTR1D-Tango | 0 | 0.609 | 0.736 | 0.101 | 0.899 | 0.589 | −0.961 | −0.503 |
| HTR1E-Tango | 0 | 0.582 | 0.744 | 0.458 | 0.690 | 0.659 | −0.056 | −0.853 |
| HTR1F-Tango | 0 | 0.384 | 0.591 | 0.208 | 1.009 | 0.451 | −0.261 | −0.838 |
| HTR2A-Tango | 0 | 0.109 | −0.063 | 0.306 | 0.856 | −0.264 | −0.791 | −0.694 |
| HTR2B-Tango | 0 | 0.351 | 0.437 | −0.094 | −0.149 | −0.613 | −0.112 | −0.889 |
| HTR2C-Tango | 0 | −0.196 | −0.299 | 0.093 | 0.983 | −0.140 | −0.908 | −0.792 |
| HTR4-Tango | 0 | 0.419 | 0.349 | 0.628 | 1.408 | 0.558 | −0.445 | −0.020 |
| HTR5-Tango | 0 | 0.531 | 0.481 | 0.788 | 0.899 | 1.026 | 0.317 | −0.238 |
| HTR6-Tango | 0 | 0.308 | 0.000 | 0.354 | 1.137 | −0.199 | −0.707 | −1.368 |
| HTR7-Tango | 0 | 0.354 | 0.002 | 0.501 | 1.127 | −0.400 | −0.376 | −0.775 |
| KISSPEPTIN-R- | 0 | −0.292 | 0.050 | 0.369 | 0.187 | −0.350 | −0.538 | −0.066 |
| Tango | | | | | | | | |
| LHCGR-Tango | 0 | 0.231 | 0.088 | −0.422 | 0.520 | 0.083 | −0.457 | −0.989 |
| LPAR1-Tango | 0 | 0.539 | 0.630 | 0.407 | 1.019 | 0.359 | 0.040 | −0.836 |
| LPAR2-Tango | 0 | 0.069 | −0.226 | −0.107 | 0.314 | 0.166 | −0.359 | −0.455 |
| LPAR4-Tango | 0 | 0.369 | 0.175 | 0.618 | 0.867 | 0.538 | −0.480 | −1.139 |
| LPAR5-Tango | 0 | −0.079 | 0.282 | 0.166 | 1.396 | −0.043 | −0.716 | −1.064 |
| LPAR6-Tango | 0 | 0.244 | −0.057 | 0.480 | 0.614 | 0.220 | −0.445 | −1.739 |
| LTB4R-Tango | 0 | 0.023 | −0.124 | 0.355 | 0.616 | 0.260 | −0.755 | −1.317 |
| LTB4R2B-Tango | 0 | 0.442 | 0.475 | 0.110 | 0.089 | 0.544 | −0.275 | −0.696 |
| MAS1-Tango | 0 | 0.088 | 0.239 | −0.291 | 0.415 | 0.088 | −0.109 | −0.234 |
| MAS1L-Tango | 0 | −0.131 | −0.231 | −0.184 | −0.338 | −0.207 | −0.204 | −1.013 |
| MC1R-Tango | 0 | −0.304 | 0.095 | −0.135 | −0.230 | −0.280 | −0.897 | −0.484 |
| MC2R-Tango | 0 | 0.151 | −0.019 | 0.600 | −0.221 | 0.184 | −0.255 | −0.627 |
| MC3R-Tango | 0 | 0.331 | 0.308 | 0.140 | 0.916 | −0.097 | −0.862 | −1.611 |
| MC4R-Tango | 0 | 1.031 | 0.606 | 0.511 | 1.212 | 0.873 | −0.188 | −0.490 |
| MC5R-Tango | 0 | 0.576 | 0.564 | 0.622 | 1.049 | 0.819 | 0.320 | −0.385 |
| MCHR1-Tango | 0 | −1.293 | −0.929 | −0.199 | 0.971 | 0.051 | −0.276 | −1.436 |
| MCHR2-Tango | 0 | −0.413 | −0.436 | 0.189 | −0.075 | −0.288 | −0.569 | −1.196 |
| MLNR-Tango | 0 | −0.304 | −0.156 | 0.049 | 0.000 | −0.429 | −0.173 | −0.077 |
| MRGPRD-Tango | 0 | −0.049 | 0.116 | 0.059 | 0.446 | 0.157 | −0.407 | −1.314 |
| MRGPRE-Tango | 0 | −0.370 | −0.271 | −0.080 | 0.649 | 0.186 | −0.406 | −0.489 |
| MRGPRF-Tango | 0 | 0.111 | 0.302 | 0.297 | 1.102 | 0.860 | −0.111 | −1.202 |
| MRGPRG-Tango | 0 | −0.556 | −0.738 | −0.263 | −0.253 | −0.343 | −0.382 | −1.061 |
| MRGPRX1-Tango | 0 | −0.681 | −0.633 | −0.141 | 1.284 | 0.075 | −1.435 | −1.460 |
| MRGPRX2-Tango | 0 | −0.237 | 0.050 | 0.374 | 0.458 | −0.182 | −0.807 | −1.871 |
| MRGPRX3-Tango | 0 | 0.074 | 0.131 | 0.378 | 0.193 | −0.222 | −0.596 | −1.101 |
| MRGPRX4-Tango | 0 | −0.365 | 0.045 | −0.293 | 0.678 | −0.520 | −0.984 | −1.173 |
| MTNR1A-Tango | 0 | 0.440 | 0.339 | −0.060 | 0.730 | 0.610 | 0.125 | −0.845 |
| MTNR1B-Tango | 0 | −0.076 | 0.014 | −0.009 | 2.155 | 0.753 | −0.150 | −1.114 |
| NMBR-Tango | 0 | −0.261 | −0.274 | 0.600 | 0.800 | 0.579 | 0.063 | −0.664 |
| NMUR1-Tango | 0 | 0.591 | 0.528 | −0.252 | 0.827 | 0.711 | 0.000 | −0.160 |
| NMUR2-Tango | 0 | 0.878 | 1.141 | 0.528 | −0.070 | 0.436 | −0.771 | −0.037 |
| NPBW1-Tango | 0 | 0.731 | 0.880 | −0.243 | 0.494 | −0.055 | −0.935 | −0.267 |
| NPBW2-Tango | 0 | 0.727 | 0.788 | −0.807 | 1.290 | 0.218 | −0.382 | −1.199 |
| NPFF1-Tango | 0 | −0.848 | −1.016 | −0.128 | −0.186 | 0.000 | −1.467 | −0.800 |
| NPFF2-Tango | 0 | 0.586 | 0.445 | −0.146 | 0.227 | 0.184 | −0.141 | −0.693 |
| NPS-Tango | 0 | −0.576 | −0.351 | 0.067 | 0.396 | 0.551 | −0.616 | −0.915 |
| NPY1R-Tango | 0 | −0.094 | −0.076 | 0.051 | 0.650 | 0.804 | −0.577 | −0.830 |
| NPY2R-Tango | 0 | −0.223 | 0.098 | −0.032 | 0.206 | −0.047 | −0.188 | −0.761 |
| NPY4R-Tango | 0 | 0.060 | 0.218 | −0.077 | −0.202 | −0.058 | −0.176 | 0.035 |
| NPY5R-Tango | 0 | 0.327 | 0.184 | −0.424 | 0.981 | 0.105 | −0.597 | −1.037 |
| NTSR1-Tango | 0 | 0.201 | 0.335 | −0.583 | 0.003 | 0.360 | −0.382 | −0.755 |
| NTSR2-Tango | 0 | 0.188 | 0.690 | 0.112 | 0.695 | 0.358 | −0.883 | −0.781 |
| OPN3-Tango | 0 | 0.136 | 0.213 | 0.097 | 0.137 | 0.156 | −0.435 | 0.015 |
| OPN5-Tango | 0 | 0.736 | 0.463 | −0.846 | 1.310 | 0.604 | −0.692 | −1.177 |
| OPRD1-Tango | 0 | −0.026 | −0.065 | −0.636 | 0.883 | 0.414 | −1.290 | −0.707 |
| OPRK1-Tango | 0 | −0.404 | −0.580 | 0.407 | 0.161 | 0.323 | −0.397 | −1.041 |
| OPRL1-Tango | 0 | −0.693 | −0.259 | 0.291 | 0.000 | −0.127 | −0.071 | −0.252 |
| OPRM1-Tango | 0 | 0.067 | 0.115 | 0.063 | 1.062 | 0.097 | −0.445 | −0.082 |
| OXER1-Tango | 0 | −0.292 | −0.356 | −0.020 | −0.102 | −0.499 | −0.217 | −0.334 |
| OXGR1-Tango | 0 | −0.097 | 0.159 | −0.426 | 0.940 | 1.218 | −0.645 | −0.578 |
| OXTR-Tango | 0 | 0.395 | 0.384 | 0.341 | 0.042 | −0.450 | −0.821 | −0.045 |
| P2RY1-Tango | 0 | 0.418 | 0.569 | −0.780 | 0.396 | −0.051 | −0.666 | −1.328 |
| P2RY10-Tango | 0 | 0.118 | 0.333 | −0.442 | 0.587 | 0.230 | −0.639 | −0.598 |
| P2RY11-Tango | 0 | 0.192 | 0.276 | −0.645 | 0.415 | 0.000 | −0.576 | −0.542 |
| P2RY12-Tango | 0 | 0.355 | 0.355 | 0.223 | 0.929 | 0.213 | −0.724 | −0.519 |
| P2RY13-Tango | 0 | 0.250 | 0.411 | 0.121 | 0.723 | 0.590 | −0.154 | −0.549 |
| P2RY14-Tango | 0 | −0.065 | −0.200 | −0.163 | 0.162 | −0.215 | −0.366 | −0.893 |
| P2RY2-Tango | 0 | 0.199 | −0.022 | −0.551 | 0.235 | −0.512 | −0.585 | −0.801 |
| P2RY4-Tango | 0 | 0.404 | 0.355 | −0.071 | 0.218 | −0.126 | −0.692 | −0.649 |
| P2RY6-Tango | 0 | −0.077 | −0.095 | −0.277 | 0.216 | 0.131 | −0.447 | −0.829 |
| P2RY8-Tango | 0 | −0.237 | −0.369 | −0.409 | 0.480 | −0.069 | −0.292 | −0.470 |
| PK1-Tango | 0 | −0.397 | −0.224 | 0.368 | 0.284 | −0.115 | −0.709 | −0.495 |
| PK2-Tango | 0 | 0.181 | −0.357 | −0.317 | 0.565 | −0.030 | −0.181 | −0.100 |
| PRRP-Tango | 0 | 0.447 | 0.386 | −0.241 | 0.621 | 0.746 | −0.089 | −0.202 |
| PTAFR-Tango | 0 | 0.381 | 0.350 | −0.432 | 0.149 | −0.278 | −0.649 | −1.153 |
| PTGDR-Tango | 0 | −0.095 | 0.168 | −0.236 | 0.662 | −0.015 | −1.002 | −0.776 |
| PTGER1-Tango | 0 | 0.427 | 0.188 | 0.342 | 0.542 | 0.115 | −0.523 | −0.118 |
| PTGER2-Tango | 0 | 0.000 | 0.292 | −0.077 | 0.547 | 0.458 | −0.050 | −0.193 |
| PTGER3-Tango | 0 | 0.552 | 0.242 | 0.135 | 1.344 | 0.621 | −0.330 | −0.882 |
| PTGER4-Tango | 0 | 0.546 | 0.073 | 0.650 | 0.546 | 0.367 | −0.532 | −1.022 |
| PTGFR-Tango | 0 | 0.646 | 0.305 | −0.200 | 0.047 | 0.580 | −0.458 | −0.730 |
| PTGIR-Tango | 0 | 0.363 | 0.628 | 0.048 | 0.877 | 0.593 | −1.498 | −1.394 |
| PTH1R-Tango | 0 | 0.160 | −0.026 | −0.018 | 1.093 | 0.109 | −0.280 | −0.692 |
| PTH2R-Tango | 0 | 0.136 | 0.028 | −0.525 | 0.720 | 0.185 | −0.343 | −1.165 |
| QRFP-Tango | 0 | 0.234 | 0.000 | −0.220 | −0.175 | −0.092 | −0.619 | −0.549 |
| RXFP1-Tango | 0 | −0.419 | −0.157 | −0.514 | 0.022 | 0.022 | −0.754 | −1.277 |
| RXFP2-Tango | 0 | −0.060 | −0.097 | −0.651 | −0.190 | −0.695 | −0.300 | −1.033 |
| RXFP3-Tango | 0 | −0.046 | −0.159 | −0.283 | 0.101 | 0.548 | −0.688 | −0.479 |
| RXFP4-Tango | 0 | 0.507 | −0.165 | −0.122 | −0.054 | 0.315 | −0.156 | −0.705 |
| S1PR1-Tango | 0 | 0.707 | 0.622 | −0.355 | 1.253 | 0.818 | −0.033 | −0.937 |
| S1PR2-Tango | 0 | 0.648 | 0.747 | 0.323 | 0.930 | 0.234 | −0.531 | −1.529 |
| S1PR3-Tango | 0 | 0.560 | 0.624 | 0.059 | 0.295 | 0.295 | −0.018 | −0.276 |
| S1PR4-Tango | 0 | 0.397 | 0.307 | −0.644 | 1.331 | 1.054 | −0.762 | −1.014 |
| S1PR5-Tango | 0 | 0.627 | 0.720 | −0.707 | 0.754 | 1.064 | −0.627 | −0.258 |
| SCTR-Tango | 0 | 0.370 | 0.260 | 0.146 | 0.297 | 0.225 | −0.685 | −0.563 |
| SSTR1-Tango | 0 | 0.285 | −0.142 | 0.103 | 0.591 | 0.145 | 0.127 | −0.189 |
| SSTR2-Tango | 0 | 0.081 | 0.428 | 0.591 | 0.830 | 0.395 | −0.181 | −0.878 |
| SSTR3-Tango | 0 | 0.745 | −0.062 | 0.445 | 0.372 | 0.878 | −0.819 | −0.415 |
| SSTR4-Tango | 0 | 0.108 | 0.292 | 0.293 | 0.853 | 0.690 | −1.267 | −0.756 |
| SSTR5-Tango | 0 | 0.385 | −0.165 | 0.754 | 0.771 | 1.234 | 0.213 | −0.310 |
| SUCNR1-Tango | 0 | −0.546 | −0.117 | −0.120 | 1.408 | 3.755 | −0.798 | −0.646 |
| TA1-Tango | 0 | 0.272 | 0.060 | −0.114 | 0.761 | −0.057 | −0.362 | −0.981 |
| TAAR2-Tango | 0 | −0.107 | 0.017 | −0.455 | 0.147 | 0.321 | −0.414 | −1.027 |
| TAAR5-Tango | 0 | −0.716 | −0.756 | −0.066 | −0.141 | −0.208 | −0.939 | −0.130 |
| TAAR6-Tango | 0 | −0.424 | −0.716 | −0.010 | −0.188 | −0.290 | −0.030 | −1.116 |
| TAAR8-Tango | 0 | −0.205 | −0.465 | −0.205 | 0.176 | 0.317 | 0.064 | −0.361 |
| TAAR9-Tango | 0 | −0.192 | −0.365 | −0.129 | −0.176 | −0.288 | −0.110 | −0.464 |
| TACR1-Tango | 0 | −0.064 | −0.241 | −0.240 | 0.140 | −0.422 | −0.085 | −1.207 |
| TACR2-Tango | 0 | 0.204 | 0.155 | −0.021 | 0.495 | 0.262 | −0.965 | −0.948 |
| TACR3-Tango | 0 | 0.435 | 0.427 | 0.067 | 0.841 | 0.503 | −0.943 | −1.376 |
| TBXA2R-Tango | 0 | 0.150 | −0.022 | 0.087 | 0.019 | −0.218 | −0.742 | −0.315 |
| TSHR-Tango | 0 | −0.378 | −0.294 | −0.423 | −0.184 | −0.703 | 0.004 | −0.996 |
| UTS2R-Tango | 0 | −0.065 | 0.194 | 0.000 | 0.719 | 0.410 | −0.260 | −0.260 |
| VIPR1-Tango | 0 | −0.920 | −1.130 | 0.184 | 0.939 | 0.191 | −0.759 | −1.689 |
| VIPR2-Tango | 0 | 0.829 | 0.803 | 0.250 | 0.050 | 0.220 | −1.527 | −1.293 |

| PRESTO-Tango | Blautia | Faecalibacterium | Oscillospira | Oscillospira | (Lachnospiraceae) | (Clostridiales) | Lactobacillus | Lactobacillus |
|---|---|---|---|---|---|---|---|---|
| with GMM | producta | prausnitzii | spp-1# | spp-2# | UC/UC | UC/UC | reuteri-1# | reuteri-2# |
| ADCYAP1R1- | 0.246 | −0.297 | −0.210 | 0.017 | −0.135 | −1.152 | −0.232 | −0.442 |
| Tango | | | | | | | | |
| ADORA1-Tango | 0.026 | −0.475 | 0.220 | 0.261 | −0.839 | −1.784 | −1.125 | −0.934 |
| ADORA2A-Tango | −0.081 | −0.152 | −0.227 | −0.095 | −0.877 | −1.168 | −0.762 | −1.398 |
| ADORA2B-Tango | −0.138 | 0.068 | −0.145 | −0.339 | −0.417 | −0.978 | −0.675 | −0.831 |
| ADORA3-Tango | 0.321 | 0.112 | −0.178 | −0.252 | 0.219 | −0.473 | −0.342 | −0.150 |
| ADRA1A-Tango | −0.117 | −0.127 | −0.410 | −0.347 | −0.239 | −0.900 | −0.427 | −0.771 |
| ADRA1B-Tango | −0.459 | −0.990 | −0.161 | −0.174 | −0.473 | −0.753 | −0.304 | −0.597 |
| ADRA1D-Tango | −0.621 | −1.196 | −0.490 | −1.037 | 0.033 | −0.410 | −0.244 | −0.666 |
| ADRA2A-Tango | 0.321 | 0.196 | −0.145 | 0.247 | −0.727 | −2.201 | −1.535 | −1.365 |
| ADRA2B-Tango | −1.241 | −0.553 | −0.621 | −0.593 | −1.064 | −2.262 | −1.103 | −1.038 |
| ADRA2C-Tango | −0.347 | −0.163 | 0.427 | 0.261 | −0.525 | −1.158 | −0.525 | −1.550 |
| ADRB1-Tango | 0.323 | −0.055 | 0.068 | 0.853 | −0.705 | −0.588 | −0.569 | −0.786 |
| ADRB2-Tango | −0.948 | −0.160 | −0.263 | −0.196 | 0.000 | −1.019 | 0.078 | 0.000 |
| ADRB3-Tango | −0.374 | 0.127 | 0.242 | 0.025 | −0.467 | −0.394 | −0.405 | −0.288 |
| AGTR1-Tango | −0.307 | −0.012 | 0.312 | 0.383 | −0.302 | −0.952 | −0.302 | −0.045 |
| AGTR2-Tango | 0.392 | −0.310 | 0.175 | 0.371 | 0.150 | −0.636 | 0.088 | 0.271 |
| APJ-Tango | 0.488 | 0.341 | −0.552 | −1.275 | 0.071 | −0.957 | −0.294 | −0.258 |
| AVPR1A-Tango | −0.695 | 0.614 | −0.328 | −1.002 | −0.082 | −1.492 | −0.285 | −0.317 |
| AVPR1B-Tango | −0.078 | −0.027 | 0.248 | −0.239 | −0.734 | −0.731 | −0.716 | −1.276 |
| AVPR2-Tango | −0.438 | 0.202 | −0.109 | −0.377 | −0.918 | −1.335 | −1.474 | −1.014 |
| BB3-Tango | −0.451 | −0.469 | −0.351 | −0.083 | −1.141 | −0.987 | −1.534 | −1.472 |
| BDKBR1-Tango | 0.107 | 0.004 | 0.021 | 0.225 | −0.822 | −0.581 | −1.186 | −0.926 |
| BDKBR2-Tango | 0.827 | 0.591 | −0.161 | 0.009 | 0.284 | −0.699 | −0.210 | −0.033 |
| C3AR1-Tango | 0.289 | 0.395 | −0.427 | −0.332 | −0.703 | −0.852 | −0.717 | −0.505 |
| C5A-Tango | 0.579 | −0.201 | 0.103 | −0.370 | 0.731 | −0.324 | −0.350 | −0.304 |
| CALCRb-Tango | 0.036 | 0.065 | −0.254 | −0.619 | −0.090 | −0.676 | −0.160 | −0.786 |
| CALCRL-Tango | −0.311 | −0.037 | −0.190 | −0.240 | −0.221 | −0.455 | −0.221 | −0.359 |
| CASR-Tango | −0.527 | −0.674 | 0.036 | −0.398 | −0.403 | −0.168 | −0.543 | −0.843 |
| CCKAR-Tango | 0.241 | −0.094 | 0.025 | −0.205 | −0.314 | −0.409 | −0.181 | −0.237 |
| CCKBR-Tango | −0.384 | −0.200 | −0.081 | −0.486 | −0.424 | −0.741 | −0.404 | −0.273 |
| CCR10-Tango | −1.073 | −0.102 | −0.251 | −0.935 | −0.567 | −1.023 | −0.807 | −0.653 |
| CCR2-Tango | −0.171 | 0.086 | −0.577 | −1.262 | −0.071 | −0.899 | −0.756 | −0.411 |
| CCR3-Tango | −0.341 | −0.476 | −0.455 | −1.046 | 0.467 | −1.613 | −0.217 | 0.000 |
| CCR4-Tango | −0.216 | −0.666 | −0.563 | −0.818 | 0.234 | −0.650 | −0.074 | −0.138 |
| CCR5-Tango | 0.109 | −0.110 | 0.000 | −0.593 | 0.226 | −0.315 | 0.154 | −0.364 |
| CCR6-Tango | 0.174 | 0.153 | −0.435 | −0.210 | 1.029 | −0.927 | 0.372 | 0.143 |
| CCR7-Tango | −0.091 | −0.595 | −0.713 | −1.033 | −0.249 | −0.398 | −0.385 | −0.595 |
| CCR8-Tango | −0.640 | −0.575 | −0.481 | −1.042 | −0.126 | −0.676 | −0.626 | −0.664 |
| CCRL2-Tango | 0.437 | −0.318 | 0.350 | 0.556 | −0.363 | −1.189 | −0.327 | −0.463 |
| CD97-Tango | −0.235 | −0.300 | 0.680 | 1.462 | −0.569 | −1.190 | −0.785 | −0.728 |
| CHRM1-Tango | −0.110 | −0.023 | 0.503 | 0.364 | −0.485 | −0.995 | −1.177 | −0.645 |
| CHRM2-Tango | −0.140 | −0.188 | 0.395 | 0.829 | −0.912 | −0.827 | −1.405 | −1.429 |
| CHRM3-Tango | −0.913 | −0.747 | 0.226 | 0.321 | −0.794 | −0.838 | −0.733 | −1.159 |
| CHRM4-Tango | 0.000 | 0.031 | 0.052 | 0.437 | −0.447 | −0.333 | −0.779 | −0.619 |
| CHRM5-Tango | 0.664 | −0.173 | −0.131 | 0.281 | −0.162 | −0.158 | −0.495 | −0.559 |
| CMKLR1-Tango | 0.438 | 0.458 | −0.079 | 0.114 | −0.384 | −0.411 | −0.360 | −0.042 |
| CNR1-Tango | −0.350 | −0.284 | −0.081 | −0.565 | −0.242 | −0.180 | −0.515 | −1.137 |
| CNR2-Tango | 0.000 | −0.116 | −0.333 | −0.464 | −0.239 | −0.628 | −0.392 | −0.072 |
| CRHR1-Tango | −0.491 | −0.491 | −0.257 | 0.357 | −0.196 | −0.727 | 0.260 | −0.477 |
| CRHR2-Tango | 0.109 | −0.024 | −0.153 | −0.484 | −0.141 | −0.359 | −0.674 | −0.925 |
| CX3C1-Tango | 0.288 | 0.733 | −0.781 | −0.728 | −0.674 | −0.620 | −1.019 | −0.769 |
| CXCR1-Tango | −0.611 | −0.280 | 0.212 | −0.104 | −0.777 | −0.857 | −0.906 | −0.258 |
| CXCR2-Tango | −0.235 | −0.462 | −0.264 | −0.023 | −0.369 | −0.429 | −0.360 | −0.203 |
| CXCR3-Tango | −0.013 | 0.149 | 0.111 | 0.393 | −0.131 | −0.914 | −0.293 | −0.169 |
| CXCR4-Tango | 0.694 | −0.009 | 0.295 | 0.695 | −0.007 | −0.555 | −0.553 | −0.231 |
| CXCR5-Tango | 0.409 | 0.172 | 0.128 | 0.409 | −0.310 | −0.671 | −0.263 | −0.276 |
| CXCR6-Tango | 0.371 | −0.103 | 0.287 | −0.153 | −0.534 | −0.371 | −0.748 | −0.784 |
| CXCR7-Tango | −0.822 | −0.793 | 0.204 | −0.761 | −0.637 | −1.037 | −1.009 | −0.809 |
| CYSLTR1-Tango | −1.111 | −0.726 | −0.191 | −0.735 | −0.290 | −1.067 | −0.637 | −0.364 |
| CYSLTR2-Tango | −0.778 | −0.748 | 0.568 | −0.189 | −0.742 | −0.826 | −0.508 | −0.485 |
| DRD1-Tango | −0.649 | −0.212 | 0.280 | 0.280 | −0.215 | −1.164 | −0.689 | −0.689 |
| DRD2-Tango | 1.803 | −0.562 | 0.216 | 0.144 | −1.570 | −1.259 | −1.932 | −1.357 |
| DRD3-Tango | 0.610 | −0.836 | 2.161 | 1.938 | −2.857 | −1.348 | −2.979 | −3.481 |
| DRD4-Tango | −0.098 | −1.477 | 0.834 | 1.274 | −2.932 | −0.672 | −3.047 | −2.867 |
| DRD5-Tango | −0.055 | −0.128 | −0.518 | −0.713 | −0.210 | −1.014 | −0.508 | −0.646 |
| EDNRA-Tango | 0.254 | −0.095 | 0.375 | −0.278 | −0.205 | −0.148 | −0.074 | −0.045 |
| EDNRB-Tango | 0.108 | 0.126 | 0.081 | 0.501 | −0.458 | −0.590 | −0.753 | −0.605 |
| ELTD1-Tango | −0.447 | −0.835 | 0.172 | 0.719 | −0.694 | −1.030 | −0.723 | −0.342 |
| F2R-Tango | 0.182 | 0.145 | 0.127 | 0.210 | −0.075 | −0.276 | −0.070 | −0.233 |
| F2RL1-Tango | 0.048 | −0.052 | −0.042 | 0.023 | −0.163 | −0.205 | −0.221 | −0.302 |
| F2RL2-Tango | −0.048 | −0.385 | 0.061 | 0.219 | −0.793 | −0.304 | −0.667 | −0.643 |
| F2RL3-Tango | −0.255 | −0.209 | 0.042 | −0.298 | −0.313 | −0.283 | −0.309 | −0.318 |
| FFA1-Tango | 0.240 | −0.080 | 0.193 | 0.319 | −0.347 | −0.520 | −0.339 | −0.009 |
| FFA2-Tango | −0.204 | −0.127 | 0.375 | 0.188 | −0.491 | −0.987 | −0.504 | −0.732 |
| FFA3-Tango | 0.093 | 0.163 | 0.409 | 0.243 | −0.371 | −0.246 | −0.618 | −0.451 |
| FPR1-Tango | 0.494 | 0.535 | 0.975 | 0.956 | 0.185 | −0.850 | −1.458 | −0.970 |
| FPR2-Tango | 0.101 | 0.263 | 0.745 | 0.732 | −0.157 | 1.656 | −1.231 | −1.397 |
| FPR3-Tango | 0.566 | −0.027 | 0.440 | 0.753 | −0.431 | −0.669 | −0.626 | −0.576 |
| FSHR-Tango | 0.075 | −0.218 | 0.305 | 0.503 | −0.705 | −0.591 | −0.548 | −0.553 |
| GABBR1-Tango | 0.274 | −0.218 | 0.281 | 0.424 | −0.895 | −0.711 | −0.895 | −1.337 |
| GAL1-Tango | −0.103 | −0.229 | 0.455 | 1.284 | −0.331 | −2.280 | −1.255 | −0.856 |
| GAL2-Tango | 0.494 | 0.214 | −0.347 | 0.171 | −0.122 | −1.208 | −0.772 | −0.585 |
| GAL3-Tango | −0.124 | 0.615 | −0.086 | −0.571 | −0.585 | −0.260 | −1.122 | −0.707 |
| GCGR-Tango | −0.020 | 0.284 | −0.591 | −0.316 | −0.503 | −0.709 | −0.267 | −0.878 |
| GHRHR-Tango | 0.424 | 0.605 | 0.017 | 0.209 | −0.814 | −0.665 | −0.892 | −0.637 |
| GHSR-Tango | 0.492 | 0.254 | −0.801 | −0.419 | −0.394 | −1.174 | −0.848 | −0.646 |
| GIPR-Tango | 0.280 | 0.323 | −0.586 | −0.547 | −0.607 | −0.997 | −1.363 | −0.809 |
| GLP1R-Tango | 0.234 | −0.126 | −0.819 | −0.859 | −0.685 | −0.368 | −1.357 | −0.618 |
| GLP2R-Tango | 0.424 | −0.068 | −0.368 | 0.062 | 0.014 | −0.605 | −0.200 | −0.014 |
| GNRHR-Tango | −0.026 | −0.065 | 0.058 | 0.206 | −0.537 | −0.877 | −0.485 | −0.628 |
| GPBA-Tango | −0.993 | −0.404 | 0.086 | 0.393 | −1.048 | −0.946 | −0.900 | −0.585 |
| GPER-Tango | 0.097 | 0.040 | 0.129 | 0.209 | −0.284 | −0.657 | −0.569 | −0.511 |
| GPR1-Tango | −0.249 | −0.194 | 0.099 | 0.552 | −0.917 | −0.605 | −1.179 | −0.808 |
| GPR101-Tango | 0.607 | 0.080 | −0.242 | 0.337 | −0.817 | −0.341 | −0.792 | −0.776 |
| GPR110-Tango | −0.141 | −0.687 | −0.449 | −0.373 | −0.798 | −0.579 | −0.765 | −0.716 |
| GPR111-Tango | 0.012 | −0.177 | −0.125 | −0.060 | −1.273 | −0.703 | −0.968 | −1.211 |
| GPR113-Tango | −0.387 | −0.387 | −0.345 | −0.134 | −0.256 | −0.686 | −0.209 | −0.600 |
| GPR114-Tango | 0.155 | −0.262 | −0.154 | −0.020 | −0.326 | −0.830 | −0.479 | −0.526 |
| GPR115-Tango | 0.039 | −0.637 | −0.106 | 0.118 | −0.441 | −0.720 | −0.418 | −0.358 |
| GPR116-Tango | −0.389 | −0.334 | 0.125 | 0.263 | −0.452 | −1.116 | −0.518 | −0.407 |
| GPR119-Tango | −0.021 | −0.387 | 0.200 | −0.006 | −0.492 | −1.178 | −0.599 | −0.654 |
| GPR12-Tango | 0.120 | −0.015 | −0.380 | −0.565 | −0.680 | −0.230 | −0.396 | −0.848 |
| GPR120-Tango | −0.204 | −0.230 | −0.066 | 0.505 | −0.747 | −0.548 | −0.541 | −0.622 |
| GPR123-Tango | 0.465 | 0.134 | 0.113 | −0.238 | −0.388 | −0.611 | −0.869 | −0.742 |
| GPR124-Tango | −0.535 | −0.461 | 0.337 | 0.200 | −0.162 | −0.716 | −0.414 | −0.358 |
| GPR125-Tango | −0.331 | −0.798 | 0.377 | 0.815 | −0.223 | −0.741 | −0.347 | −0.420 |
| GPR126-Tango | −0.071 | −0.088 | 0.167 | 0.258 | −0.719 | −0.801 | −0.684 | −0.902 |
| GPR132-Tango | −0.584 | −0.354 | −0.424 | −0.550 | −0.520 | −0.539 | −0.409 | −0.689 |
| GPR133-Tango | −0.067 | −0.359 | −0.043 | 0.016 | −0.403 | −1.043 | −0.349 | −0.301 |
| GPR135-Tango | −0.045 | 0.307 | −0.082 | −0.307 | −0.637 | −0.848 | −0.422 | −0.637 |
| GPR141-Tango | 0.142 | 0.055 | −0.165 | −0.039 | −0.488 | −0.321 | −0.538 | −0.235 |
| GPR142-Tango | −0.831 | −0.696 | −0.205 | −0.335 | 0.000 | −0.736 | −0.044 | −0.095 |
| GPR143-Tango | −0.753 | −0.709 | −0.256 | −0.650 | −0.046 | −0.120 | −0.724 | −0.380 |
| GPR144-Tango | −0.445 | −0.642 | −0.227 | −0.348 | −0.325 | −0.745 | −0.715 | −1.340 |
| GPR146-Tango | −1.000 | −0.047 | −0.523 | −0.263 | −0.403 | −0.723 | −0.866 | −0.335 |
| GPR148-Tango | −0.477 | −0.658 | 1.350 | 1.263 | −0.879 | −0.087 | −1.132 | −0.511 |
| GPR149-Tango | −0.515 | −0.551 | 0.107 | 0.795 | −1.016 | −1.030 | −1.569 | −1.374 |
| GPR15-Tango | −0.322 | −0.260 | −0.079 | −0.795 | −0.649 | −1.096 | −0.399 | 0.037 |
| GPR150-Tango | −0.376 | −0.093 | 0.103 | −0.082 | −0.531 | 0.204 | 0.187 | −0.267 |
| GPR151-Tango | −0.199 | −0.600 | 0.336 | 0.178 | 0.058 | −0.093 | −0.559 | 0.000 |
| GPR152-Tango | −0.183 | −0.224 | 0.189 | 0.785 | 0.090 | −0.242 | 0.221 | −0.075 |
| GPR153-Tango | 0.013 | −0.295 | 0.114 | 0.337 | −0.413 | −0.473 | −0.586 | −0.670 |
| GPR156-Tango | 0.141 | −0.170 | 0.837 | 1.681 | −0.261 | −0.913 | −0.659 | −0.611 |
| GPR157-Tango | −0.383 | −0.047 | 0.370 | 0.610 | −0.262 | −0.370 | −0.270 | −0.413 |
| GPR158-Tango | −0.093 | 0.011 | 0.338 | −0.020 | 0.121 | −0.340 | −0.155 | −0.441 |
| GPR160-Tango | −0.173 | −0.418 | −0.095 | −0.061 | −0.050 | 0.074 | −0.419 | −0.318 |
| GPR161-Tango | −0.467 | −0.743 | −0.159 | −0.208 | −0.481 | −0.445 | −0.723 | −0.518 |
| GPR162-Tango | −0.250 | −0.391 | −0.386 | −0.361 | −0.631 | −0.852 | −0.888 | −0.671 |
| GPR17-Tango | −0.696 | −0.878 | 0.942 | 0.350 | 0.073 | −0.479 | −0.036 | 0.013 |
| GPR171-Tango | −0.503 | −0.672 | 0.286 | 0.120 | −0.192 | −0.799 | −0.489 | −0.224 |
| GPR173-Tango | −0.205 | −0.365 | −0.155 | 0.160 | −0.302 | −0.499 | −0.275 | −0.615 |
| GPR174-Tango | 0.045 | −0.472 | −0.180 | −0.085 | −0.197 | −0.703 | −0.175 | −0.197 |
| GPR18-Tango | −0.220 | −0.623 | −0.110 | −0.141 | −0.017 | −0.484 | −0.997 | −0.369 |
| GPR182-Tango | −0.471 | −0.695 | −0.121 | 0.232 | −0.384 | −0.594 | −0.453 | −0.369 |
| GPR183-Tango | −0.027 | −0.211 | −0.163 | −0.025 | −0.398 | −0.516 | −0.199 | −0.316 |
| GPR19-Tango | −0.482 | −0.608 | −0.258 | −0.055 | −0.448 | −0.890 | −0.103 | −0.023 |
| GPR20-Tango | 0.000 | 0.118 | −0.037 | −0.222 | −0.585 | −0.523 | −0.322 | −0.074 |
| GPR21-Tango | −0.078 | −0.019 | −0.056 | 0.236 | −0.787 | −0.511 | −1.102 | −0.601 |
| GPR22-Tango | 0.130 | −0.624 | −0.175 | 0.228 | −0.406 | −0.297 | −0.602 | −0.862 |
| GPR25-Tango | −0.426 | 0.016 | 0.087 | −0.252 | −0.429 | −0.070 | −0.284 | −0.626 |
| GPR26-Tango | 0.483 | 0.137 | 0.048 | −0.614 | −0.034 | −0.225 | 0.034 | −0.118 |
| GPR27-Tango | −0.244 | −0.227 | 0.205 | 0.057 | −0.201 | −0.081 | −0.511 | −0.609 |
| GPR3-Tango | 0.365 | −0.124 | 0.203 | −0.398 | −0.301 | 0.220 | −0.349 | −0.393 |
| GPR31-Tango | −0.283 | −0.132 | 0.051 | −0.130 | −0.412 | −0.576 | −0.402 | −0.264 |
| GPR32-Tango | 0.040 | 0.097 | −0.131 | −0.237 | −0.300 | −0.794 | −1.383 | −0.300 |
| GPR34-Tango | −0.035 | −0.614 | 0.146 | 0.092 | −1.040 | −0.508 | −0.954 | −0.954 |
| GPR35-Tango | −0.373 | 0.095 | 0.074 | −0.169 | −0.690 | −0.248 | −1.134 | −0.767 |
| GPR37-Tango | 0.104 | −0.215 | 0.260 | 0.364 | −0.863 | −0.525 | −1.054 | −1.014 |
| GPR37L1-Tango | 0.184 | −0.198 | 0.262 | 0.432 | −0.097 | −0.622 | −0.285 | −0.548 |
| GPR39-Tango | −0.044 | −0.062 | −0.190 | −0.270 | −0.298 | 0.111 | −0.586 | −0.214 |
| GPR4-Tango | −0.041 | −0.154 | −0.173 | −0.300 | −0.861 | −0.094 | −0.872 | −0.815 |
| GPR44-Tango | 0.279 | 0.313 | −0.118 | −0.214 | −0.609 | −0.371 | −0.823 | −0.823 |
| GPR45-Tango | 0.110 | −0.097 | −0.649 | −0.321 | −0.054 | −0.951 | −0.525 | −0.525 |
| GPR50-Tango | 0.549 | 0.080 | 0.090 | −0.222 | −0.051 | −0.181 | −0.177 | 0.025 |
| GPR52-Tango | 0.461 | 0.125 | −0.051 | 0.201 | −0.817 | 0.062 | −1.331 | −0.469 |
| GPR55-Tango | 0.053 | −0.055 | 0.495 | 0.141 | −1.267 | −0.461 | −1.367 | −0.688 |
| GPR56-Tango | −0.146 | −0.049 | −0.059 | 0.311 | −0.944 | −0.513 | −0.763 | −0.659 |
| GPR6-Tango | 0.117 | −0.229 | 0.194 | 0.203 | −0.443 | −0.321 | −0.761 | −0.305 |
| GPR61-Tango | 0.156 | 0.005 | 0.018 | 0.072 | −0.497 | 0.339 | −0.805 | −0.984 |
| GPR62-Tango | −0.023 | 0.125 | 0.042 | 0.194 | −0.330 | −0.914 | −0.330 | −0.477 |
| GPR63-Tango | −0.212 | −0.513 | 0.083 | 0.306 | −0.465 | −0.901 | −0.766 | −0.622 |
| GPR64-Tango | −0.117 | −0.404 | 0.039 | 0.346 | −0.571 | −1.001 | −0.843 | −0.862 |
| GPR65-Tango | 0.163 | −0.116 | 0.150 | 0.284 | −0.462 | −0.522 | −0.663 | −0.815 |
| GPR68-Tango | −0.066 | −0.202 | −0.106 | −0.037 | −0.974 | −0.868 | −0.877 | −1.182 |
| GPR75-Tango | 0.024 | −0.028 | −0.185 | 0.169 | −0.377 | −0.918 | −0.797 | −0.658 |
| GPR77-Tango | −0.223 | −0.161 | −0.185 | −0.366 | −0.977 | −1.055 | −0.985 | −1.385 |
| GPR78-Tango | 0.485 | −0.025 | 0.381 | 0.235 | −0.226 | −0.805 | −0.334 | −0.841 |
| GPR82-Tango | 0.010 | −0.123 | −0.263 | 0.103 | −0.499 | −0.884 | −0.561 | −0.898 |
| GPR83-Tango | −0.357 | −0.252 | −0.117 | −0.151 | −0.584 | −0.335 | −0.544 | −0.400 |
| GPR84-Tango | −0.708 | −0.606 | −0.162 | −0.065 | −0.792 | −0.485 | −0.588 | −0.703 |
| GPR85-Tango | −0.525 | −0.438 | 0.064 | 0.322 | −0.448 | −0.730 | −0.577 | −0.577 |
| GPR87-Tango | −0.129 | −0.280 | 0.206 | 0.430 | −0.290 | −0.467 | −0.420 | −0.354 |
| GPR88-Tango | −0.436 | −0.203 | 0.096 | 0.350 | −0.635 | −0.691 | −0.759 | −0.635 |
| GPR97-Tango | −0.823 | −0.550 | −0.089 | −0.101 | −0.593 | −1.005 | −0.751 | −0.607 |
| GPRC5A-Tango | −0.478 | −0.311 | 0.183 | 0.756 | −0.201 | −0.976 | −0.668 | −0.457 |
| GPRC5B-Tango | −0.475 | −0.742 | 0.122 | 0.405 | −0.408 | −0.262 | −0.290 | −0.450 |
| GPRC5C-Tango | −0.467 | −0.728 | −0.186 | 0.230 | −0.143 | −0.514 | −0.098 | −0.425 |
| GPRC5D-Tango | −0.238 | −0.354 | 0.232 | 0.066 | −0.641 | −0.822 | −0.647 | −0.598 |
| GPRC6A-Tango | −0.278 | −0.538 | −0.037 | 0.216 | −0.495 | −0.505 | −0.420 | −0.388 |
| GRM1-Tango | −0.262 | −0.461 | −0.450 | −0.362 | −0.135 | −0.687 | −0.346 | −0.234 |
| GRM2-Tango | −0.520 | −0.439 | −0.200 | −0.015 | −1.034 | −0.793 | −1.055 | −0.903 |
| GRM4-Tango | −0.245 | −0.343 | −0.684 | −0.379 | −0.642 | −0.086 | −0.668 | −0.577 |
| GRM5-Tango | −0.305 | −0.511 | −0.355 | −0.153 | −0.875 | −0.596 | −0.885 | −1.070 |
| GRM6-Tango | −0.656 | −0.263 | −0.493 | −0.115 | −0.560 | −0.408 | −0.904 | −0.620 |
| GRM7-Tango | −0.913 | −0.792 | −0.158 | 0.161 | −0.409 | −0.876 | −0.553 | −0.466 |
| GRM8-Tango | −0.803 | −0.840 | −0.021 | 0.170 | −0.672 | −0.748 | −0.349 | −0.349 |
| GRPR-Tango | −0.246 | −0.246 | −0.659 | −0.945 | −0.026 | −0.575 | −0.551 | −0.354 |
| HCA1-Tango | −0.515 | −0.280 | −0.113 | 0.113 | −0.138 | −0.715 | −0.138 | 0.082 |
| HCA2-Tango | −0.821 | −0.724 | 0.665 | 0.369 | −0.284 | −0.149 | −0.453 | −0.534 |
| HCA3-Tango | −0.463 | −0.360 | 0.046 | 0.388 | −0.158 | −0.605 | 0.000 | −0.092 |
| HCTR1-Tango | 0.000 | −0.511 | 0.032 | 0.285 | −0.280 | −0.649 | 0.935 | −0.766 |
| HCTR2-Tango | 0.087 | −0.093 | 0.260 | 0.358 | −0.621 | −0.563 | 0.632 | −0.621 |
| HRH1-Tango | −0.867 | −1.517 | −0.004 | 0.270 | −0.526 | 0.060 | −0.456 | −0.168 |
| HRH2-Tango | −0.677 | −0.622 | 0.339 | 0.310 | −0.390 | −0.349 | −0.108 | 0.081 |
| HRH3-Tango | −0.990 | −0.810 | 0.309 | 0.473 | −0.561 | −0.189 | 0.648 | 0.972 |
| HRH4-Tango | −0.720 | −0.550 | 0.643 | 1.060 | −0.429 | −0.510 | 0.465 | 0.813 |
| HTR1A-Tango | −0.313 | −0.195 | 0.472 | 1.117 | −0.642 | −0.782 | −0.610 | −0.425 |
| HTR1B-Tango | −0.634 | −0.562 | 0.072 | 0.574 | −0.829 | −0.292 | −0.665 | −0.701 |
| HTR1D-Tango | −0.290 | −0.565 | 0.193 | 0.269 | −0.551 | −0.635 | −0.668 | −0.498 |
| HTR1E-Tango | −0.206 | −0.215 | 0.231 | 0.525 | −0.320 | −0.088 | −0.475 | −0.063 |
| HTR1F-Tango | −0.655 | −0.895 | −0.287 | 0.316 | −0.549 | −0.963 | −0.478 | −0.623 |
| HTR2A-Tango | −1.239 | −0.779 | 0.001 | 0.126 | −0.747 | −0.640 | −0.869 | −0.941 |
| HTR2B-Tango | −0.635 | −0.947 | 0.336 | 0.825 | −0.895 | −0.325 | −0.825 | −0.870 |
| HTR2C-Tango | −0.967 | −0.792 | −0.030 | 0.126 | −0.671 | −0.817 | −0.710 | −0.632 |
| HTR4-Tango | −0.050 | −0.020 | −0.226 | −0.219 | −0.295 | −0.568 | −0.429 | −0.842 |
| HTR5-Tango | −0.195 | 0.110 | −0.318 | −0.192 | −0.139 | −0.500 | −0.311 | −0.326 |
| HTR6-Tango | −1.071 | −0.821 | −0.096 | 0.111 | −0.573 | −0.454 | −0.790 | −0.848 |
| HTR7-Tango | −0.112 | −0.947 | 0.244 | 0.623 | −0.529 | −0.860 | −0.801 | −0.944 |
| KISSPEPTIN-R- | −0.684 | 0.181 | 0.123 | −0.142 | 0.335 | −0.671 | 0.437 | −0.700 |
| Tango | | | | | | | | |
| LHCGR-Tango | −0.927 | −1.227 | −0.195 | −0.069 | 0.381 | −0.209 | 0.135 | 0.485 |
| LPAR1-Tango | −0.268 | −0.273 | 0.252 | 0.330 | −0.175 | −0.156 | −0.272 | −0.485 |
| LPAR2-Tango | 0.000 | −0.334 | 0.124 | −0.156 | −0.100 | −0.497 | −0.637 | −0.637 |
| LPAR4-Tango | −0.998 | −1.046 | −0.081 | −0.566 | −0.071 | −0.842 | −0.778 | −0.614 |
| LPAR5-Tango | −1.408 | −0.925 | −0.154 | −0.514 | −0.137 | −0.745 | −0.341 | −0.395 |
| LPAR6-Tango | −0.817 | −1.130 | 0.002 | −0.304 | −0.145 | −0.220 | −0.260 | −0.365 |
| LTB4R-Tango | −0.667 | −0.285 | 0.009 | −0.303 | 0.287 | −0.457 | 0.077 | 0.503 |
| LTB4R2B-Tango | −0.497 | −0.671 | 0.109 | 0.037 | 0.204 | 0.059 | −0.021 | −0.289 |
| MAS1-Tango | −0.172 | −0.267 | −0.088 | 0.116 | −0.421 | −0.312 | −0.609 | −0.506 |
| MAS1L-Tango | −0.253 | −0.763 | 0.461 | 0.715 | −0.667 | −0.378 | −0.477 | −0.621 |
| MC1R-Tango | −0.804 | −0.662 | 0.204 | 0.163 | −0.614 | −0.574 | −0.684 | −0.778 |
| MC2R-Tango | 0.041 | −0.255 | 0.216 | −0.546 | −0.409 | −0.526 | −0.520 | −0.710 |
| MC3R-Tango | −0.075 | −0.710 | −0.140 | 0.064 | −0.019 | −0.126 | −0.845 | −0.131 |
| MC4R-Tango | −0.097 | −0.064 | −0.168 | 0.216 | 0.062 | −0.530 | −0.032 | −0.628 |
| MC5R-Tango | −0.061 | −0.061 | 0.208 | 0.607 | 0.240 | −0.734 | −0.473 | −0.289 |
| MCHR1-Tango | −1.173 | −1.386 | −0.337 | −0.288 | −0.050 | −0.744 | −0.326 | −0.270 |
| MCHR2-Tango | −0.605 | −0.745 | −0.167 | −0.122 | −0.719 | −0.870 | −0.810 | −0.526 |
| MLNR-Tango | −0.436 | −0.560 | 0.161 | 0.191 | −0.933 | −0.787 | −0.559 | −1.064 |
| MRGPRD-Tango | −1.261 | −1.225 | −0.303 | −0.084 | −0.329 | −0.404 | −0.529 | −0.472 |
| MRGPRE-Tango | −0.926 | −0.430 | 0.049 | 0.352 | −0.439 | −0.332 | −0.588 | −0.454 |
| MRGPRF-Tango | −0.549 | −0.365 | 0.460 | 0.874 | −0.299 | −0.527 | −0.360 | −0.402 |
| MRGPRG-Tango | 0.400 | −0.677 | 0.345 | 0.810 | −0.607 | −0.582 | −0.473 | −0.702 |
| MRGPRX1-Tango | −0.596 | −0.823 | −0.445 | 0.167 | −0.183 | −0.696 | −0.124 | −0.414 |
| MRGPRX2-Tango | −1.118 | −1.068 | −0.083 | 0.293 | −0.752 | −0.661 | −1.248 | −1.199 |
| MRGPRX3-Tango | −0.846 | −0.426 | −0.018 | −0.071 | −1.100 | −0.774 | −0.872 | −0.964 |
| MRGPRX4-Tango | −1.283 | −1.138 | −0.395 | −0.119 | −1.258 | −1.902 | −1.424 | −1.548 |
| MTNR1A-Tango | −0.168 | −0.280 | −0.572 | −0.335 | −0.729 | −0.552 | −1.318 | −1.318 |
| MTNR1B-Tango | −0.845 | −0.521 | −0.245 | 0.072 | −0.605 | −0.923 | −0.756 | −0.940 |
| NMBR-Tango | −0.255 | −0.144 | 0.192 | 0.294 | −0.241 | −1.158 | −0.427 | −0.444 |
| NMUR1-Tango | −0.341 | 0.317 | −0.379 | −0.475 | 0.222 | −0.698 | 0.328 | 0.000 |
| NMUR2-Tango | −0.664 | −0.060 | −0.974 | −1.268 | −0.229 | −0.187 | −0.124 | −0.387 |
| NPBW1-Tango | 0.281 | 0.065 | 0.435 | 1.030 | −0.640 | −1.328 | −1.225 | −2.506 |
| NPBW2-Tango | −0.482 | −0.708 | 0.386 | 1.019 | −1.746 | −1.373 | −1.323 | −1.999 |
| NPFF1-Tango | −0.047 | −0.288 | 0.110 | 0.794 | −1.110 | −1.227 | −1.303 | −1.360 |
| NPFF2-Tango | −0.096 | −0.440 | 0.110 | −0.229 | 0.027 | −0.596 | −0.065 | −0.560 |
| NPS-Tango | −0.062 | −0.351 | −0.234 | 0.100 | 0.020 | −0.793 | −0.549 | −0.225 |
| NPY1R-Tango | −0.491 | −0.546 | 0.169 | −0.108 | −0.078 | −1.012 | −0.423 | −1.078 |
| NPY2R-Tango | −0.195 | −0.471 | 0.176 | 0.301 | −1.671 | −0.608 | −1.871 | −1.522 |
| NPY4R-Tango | 0.409 | 0.382 | −0.267 | −0.030 | −0.798 | −0.842 | −0.991 | −0.954 |
| NPY5R-Tango | −0.889 | −0.968 | −0.098 | −0.199 | −0.560 | −0.445 | −1.293 | −1.382 |
| NTSR1-Tango | −0.597 | −0.664 | 0.124 | 0.146 | −0.897 | −0.730 | −1.551 | −1.108 |
| NTSR2-Tango | −0.883 | −1.037 | 0.132 | −0.127 | −0.284 | −0.939 | −0.748 | −0.797 |
| OPN3-Tango | −0.045 | −0.131 | 0.000 | −0.129 | −1.143 | −0.371 | −0.756 | −1.022 |
| OPN5-Tango | −0.884 | −0.889 | −0.198 | −0.046 | −0.879 | −0.321 | −2.103 | −1.672 |
| OPRD1-Tango | −1.061 | −0.546 | −0.144 | −0.017 | −0.280 | −1.157 | −0.995 | −0.481 |
| OPRK1-Tango | 0.293 | −0.732 | 0.419 | 0.719 | −0.167 | −0.136 | −0.182 | −0.110 |
| OPRL1-Tango | 0.034 | 0.034 | 0.348 | 0.096 | −0.756 | −0.565 | −0.127 | −0.422 |
| OPRM1-Tango | −0.190 | −0.140 | −0.383 | −0.161 | −0.640 | −0.957 | −0.493 | −0.194 |
| OXER1-Tango | 0.000 | −0.082 | 0.305 | 0.629 | −0.419 | −0.439 | −0.384 | 0.057 |
| OXGR1-Tango | −0.931 | −0.436 | −0.331 | −0.352 | −0.316 | −1.424 | −0.883 | −0.703 |
| OXTR-Tango | −0.086 | −0.245 | −0.007 | 0.101 | −1.058 | −0.862 | −1.077 | −0.758 |
| P2RY1-Tango | −1.003 | −0.824 | 0.295 | 0.837 | −1.547 | −0.749 | −1.663 | −1.035 |
| P2RY10-Tango | −0.508 | −0.349 | −0.078 | −0.113 | −1.560 | −0.546 | −1.430 | −0.515 |
| P2RY11-Tango | −0.559 | −0.482 | −0.049 | −0.036 | −0.617 | −0.752 | −0.814 | −1.043 |
| P2RY12-Tango | −0.263 | 0.571 | −0.387 | 0.372 | −1.094 | −0.822 | −1.616 | −1.906 |
| P2RY13-Tango | −0.268 | −0.343 | 0.412 | 0.412 | −0.509 | −0.421 | −0.538 | −0.530 |
| P2RY14-Tango | −0.559 | −0.551 | 0.606 | 0.466 | −1.253 | −0.380 | −1.095 | −0.905 |
| P2RY2-Tango | −0.660 | −0.711 | 0.049 | 0.400 | −1.159 | −0.566 | −1.342 | −0.962 |
| P2RY4-Tango | −0.861 | −0.567 | −0.014 | 0.182 | −1.269 | −0.806 | −1.405 | −1.057 |
| P2RY6-Tango | −0.567 | −0.771 | 0.646 | 0.868 | −0.986 | −0.425 | −1.147 | −0.786 |
| P2RY8-Tango | −0.148 | −0.370 | 0.173 | 0.149 | −0.635 | −0.408 | −0.496 | −0.573 |
| PK1-Tango | −0.655 | −0.766 | 0.072 | 0.078 | −0.094 | −0.954 | −0.588 | −0.430 |
| PK2-Tango | −0.222 | 0.071 | 0.187 | 0.176 | −0.227 | 0.061 | 0.000 | −0.176 |
| PRRP-Tango | −0.678 | −0.575 | 0.051 | 0.088 | −0.758 | −0.970 | −1.019 | −1.307 |
| PTAFR-Tango | −0.729 | −1.244 | 0.028 | 0.440 | −0.737 | −0.543 | −1.194 | −1.194 |
| PTGDR-Tango | −1.097 | −0.538 | −0.022 | 0.053 | −0.961 | −0.851 | −1.517 | −1.259 |
| PTGER1-Tango | −0.213 | 0.222 | 0.173 | −0.104 | −0.733 | −1.208 | −0.913 | −0.529 |
| PTGER2-Tango | 0.126 | 0.399 | 0.183 | 0.034 | −0.601 | −0.429 | −0.835 | −0.923 |
| PTGER3-Tango | −0.173 | −0.657 | 0.143 | 0.456 | −0.192 | −0.303 | −0.551 | −0.153 |
| PTGER4-Tango | 0.219 | −0.408 | 0.202 | 0.489 | −0.247 | −0.088 | −0.648 | −0.162 |
| PTGFR-Tango | −0.458 | −0.503 | −0.462 | −0.094 | −0.098 | −0.320 | −0.601 | −0.485 |
| PTGIR-Tango | −0.866 | −0.987 | 0.045 | 0.045 | −0.176 | −0.722 | −0.301 | −1.024 |
| PTH1R-Tango | 0.020 | −0.784 | −0.404 | −0.184 | −0.447 | −0.400 | −0.709 | −0.611 |
| PTH2R-Tango | −0.314 | −0.825 | −0.121 | 0.154 | −0.647 | −0.710 | −0.804 | −0.677 |
| QRFP-Tango | −0.224 | −0.273 | −0.515 | −0.065 | −0.154 | −0.719 | −0.280 | −0.739 |
| RXFP1-Tango | −0.233 | −0.754 | −0.183 | −0.019 | −0.344 | −0.693 | −0.430 | −0.170 |
| RXFP2-Tango | −0.009 | −0.968 | 0.181 | 0.099 | −0.825 | −0.341 | −0.257 | −0.627 |
| RXFP3-Tango | 0.118 | −0.357 | −0.521 | −0.218 | −0.410 | −0.632 | −0.277 | −0.258 |
| RXFP4-Tango | 0.471 | −0.310 | −0.611 | −0.322 | 0.000 | −0.714 | −0.685 | −0.403 |
| S1PR1-Tango | −0.322 | −0.431 | −0.313 | −0.346 | 0.419 | 0.760 | 0.397 | 0.486 |
| S1PR2-Tango | −0.721 | −0.736 | −0.453 | −0.869 | 0.357 | −0.476 | 0.054 | 0.199 |
| S1PR3-Tango | −0.234 | −0.379 | −0.605 | −0.653 | 0.598 | 0.130 | 0.295 | 0.648 |
| S1PR4-Tango | −1.385 | −0.865 | −0.470 | −0.238 | −0.152 | −0.376 | −0.089 | 0.400 |
| S1PR5-Tango | −0.346 | −0.664 | −0.114 | 0.523 | 0.229 | −0.852 | −0.220 | −0.170 |
| SCTR-Tango | 0.488 | −0.178 | 0.532 | 0.438 | 0.413 | −0.808 | −0.304 | −0.640 |
| SSTR1-Tango | 0.127 | −0.115 | −0.442 | −0.402 | −0.143 | 0.000 | −0.052 | −0.655 |
| SSTR2-Tango | −0.596 | −0.766 | 0.069 | −0.211 | 0.830 | −0.674 | 0.333 | 0.333 |
| SSTR3-Tango | −0.819 | −0.953 | 0.000 | −0.177 | 1.198 | −1.678 | 0.667 | 0.542 |
| SSTR4-Tango | −0.873 | −0.393 | 0.000 | 0.067 | −0.505 | −0.751 | −0.156 | 0.295 |
| SSTR5-Tango | −0.705 | 0.506 | 1.503 | 0.931 | −0.032 | −0.798 | 0.062 | −0.122 |
| SUCNR1-Tango | 2.561 | −0.287 | 0.860 | 0.966 | 0.088 | −1.806 | 2.206 | −0.177 |
| TA1-Tango | −0.514 | −0.849 | 0.375 | −0.020 | −0.549 | −0.342 | −0.617 | −0.859 |
| TAAR2-Tango | −0.434 | −0.708 | 0.174 | −0.198 | 0.201 | −0.534 | −0.068 | 0.027 |
| TAAR5-Tango | −0.354 | −0.849 | 0.201 | 0.753 | −0.360 | −1.336 | −0.609 | −0.807 |
| TAAR6-Tango | −0.312 | −0.899 | 0.248 | 0.605 | −0.670 | −0.864 | −0.466 | −0.422 |
| TAAR8-Tango | −0.258 | −0.471 | 0.333 | 0.836 | −0.153 | −0.551 | −0.052 | 0.077 |
| TAAR9-Tango | −0.054 | −0.338 | 0.138 | 0.088 | −0.358 | −0.207 | −0.207 | −0.333 |
| TACR1-Tango | −0.554 | −0.706 | 0.206 | 0.225 | −0.452 | −0.861 | −0.552 | −0.740 |
| TACR2-Tango | −0.153 | −0.391 | 0.268 | −0.086 | −0.282 | 0.013 | −0.392 | −0.403 |
| TACR3-Tango | −0.636 | −0.943 | 0.070 | 0.507 | −0.172 | −0.847 | −0.622 | −0.428 |
| TBXA2R-Tango | −0.263 | −0.173 | −0.380 | −0.411 | −0.531 | −0.700 | −0.264 | −0.700 |
| TSHR-Tango | −0.414 | −0.812 | −0.316 | −0.056 | −0.314 | −0.871 | −0.114 | −0.375 |
| UTS2R-Tango | 0.000 | 0.050 | −0.126 | −0.269 | 0.369 | −0.602 | 0.156 | 0.204 |
| VIPR1-Tango | −0.958 | −0.958 | 0.197 | 0.404 | −0.140 | −0.218 | −0.696 | −0.834 |
| VIPR2-Tango | −1.615 | −1.112 | 1.431 | 1.249 | −0.106 | 1.436 | −0.737 | 0.145 |

| PRESTO-Tango | Lactacillus | Pediococcus | Pediococcus | Pediococcus | Streptococcus | Streptococcus | Streptococcus | Streptococcus |
|---|---|---|---|---|---|---|---|---|
| with GMM | UC | UC-1# | UC-2# | UC-2# | luteciae | spp-1# | spp-2# | spp-3# |
| ADCYAP1R1- | −0.774 | −0.992 | −0.188 | −0.717 | −0.518 | −0.313 | −0.589 | −0.050 |
| Tango | | | | | | | | |
| ADORA1-Tango | −1.293 | −0.951 | −0.665 | −0.933 | −0.962 | −0.615 | −1.283 | −0.531 |
| ADORA2A-Tango | −0.801 | −1.163 | 0.173 | −0.304 | −0.528 | −0.240 | 0.055 | −0.413 |
| ADORA2B-Tango | −0.308 | −0.699 | −0.183 | −0.270 | −0.635 | −0.249 | −0.143 | −0.470 |
| ADORA3-Tango | −0.211 | −0.095 | −0.063 | −0.012 | −0.202 | 0.167 | 0.039 | 0.103 |
| ADRA1A-Tango | −0.137 | −0.510 | −0.125 | −0.117 | −0.643 | −0.483 | −0.535 | −0.426 |
| ADRA1B-Tango | −0.208 | −0.445 | −0.308 | 0.387 | −0.208 | 0.041 | −1.192 | 0.075 |
| ADRA1D-Tango | −0.608 | −0.523 | −0.407 | −0.058 | −0.261 | −0.173 | −0.374 | −0.218 |
| ADRA2A-Tango | −1.000 | −0.607 | −1.542 | −0.240 | −1.733 | −0.176 | 0.670 | −0.647 |
| ADRA2B-Tango | −1.735 | −1.179 | −1.336 | −0.929 | −0.650 | 0.153 | −0.491 | −0.249 |
| ADRA2C-Tango | −0.913 | −1.159 | 0.179 | −0.038 | −0.642 | 0.455 | 0.467 | −0.288 |
| ADRB1-Tango | −0.153 | −0.447 | −0.285 | −0.394 | −0.517 | −0.312 | 0.300 | 0.559 |
| ADRB2-Tango | −0.876 | −0.974 | −0.822 | −0.248 | −0.088 | −0.040 | −0.913 | 0.229 |
| ADRB3-Tango | −0.520 | −1.127 | −0.573 | 0.202 | −0.535 | −0.371 | 0.000 | −0.087 |
| AGTR1-Tango | −0.651 | −0.946 | −0.159 | 0.128 | −0.250 | −0.256 | −0.171 | −0.256 |
| AGTR2-Tango | −0.509 | −0.354 | −0.645 | −0.367 | −0.203 | 0.012 | −0.813 | −0.137 |
| APJ-Tango | −1.079 | −1.489 | −0.843 | −0.348 | −0.418 | −0.640 | −0.758 | −0.334 |
| AVPR1A-Tango | −0.510 | −0.922 | −0.284 | 0.090 | −0.286 | −0.286 | −0.836 | −0.087 |
| AVPR1B-Tango | −0.662 | −1.017 | −0.294 | 0.299 | −0.385 | −0.304 | −0.706 | 0.000 |
| AVPR2-Tango | −0.987 | −0.941 | −0.836 | −0.122 | −0.778 | −0.088 | −1.338 | −0.428 |
| BB3-Tango | −1.019 | −1.109 | −0.518 | −0.690 | −0.622 | −0.359 | −0.856 | −0.421 |
| BDKBR1-Tango | −0.596 | −0.893 | −0.098 | 0.071 | −0.454 | −0.349 | 0.015 | −0.468 |
| BDKBR2-Tango | −0.304 | −0.160 | −0.207 | 0.013 | 0.040 | −0.120 | −0.121 | 0.256 |
| C3AR1-Tango | −0.737 | −0.821 | −0.569 | −0.279 | −0.273 | −0.442 | −0.569 | −0.295 |
| C5A-Tango | −0.526 | −0.746 | −0.501 | 0.191 | −0.030 | −0.216 | −1.035 | 0.344 |
| CALCRb-Tango | −0.485 | −0.946 | −0.091 | −0.320 | −0.481 | −0.280 | −0.301 | 0.015 |
| CALCRL-Tango | −0.439 | −0.760 | 0.252 | 0.175 | −0.414 | −0.131 | 0.020 | −0.304 |
| CASR-Tango | −0.345 | −0.767 | 0.039 | 0.269 | −0.068 | −0.167 | −0.478 | 0.547 |
| CCKAR-Tango | −0.198 | −0.202 | 0.028 | 0.129 | −0.182 | −0.080 | −0.650 | 0.350 |
| CCKBR-Tango | −0.422 | −0.607 | −0.344 | −0.059 | −0.456 | −0.472 | −0.729 | −0.147 |
| CCR10-Tango | −1.000 | −1.648 | −0.280 | −0.251 | −0.470 | 0.020 | −0.818 | −0.369 |
| CCR2-Tango | −0.059 | −0.373 | −0.225 | −1.050 | −0.206 | −0.363 | −0.097 | −0.354 |
| CCR3-Tango | −0.442 | −0.497 | −0.641 | −0.956 | −0.585 | −0.494 | −0.710 | −0.010 |
| CCR4-Tango | −0.680 | −1.088 | −0.252 | −0.329 | −0.329 | 0.022 | 0.025 | 0.492 |
| CCR5-Tango | −0.171 | −0.551 | −0.324 | 0.148 | −0.231 | −0.142 | −0.161 | 0.691 |
| CCR6-Tango | −0.305 | −0.752 | −0.598 | 0.031 | 0.217 | 0.148 | −0.455 | 0.847 |
| CCR7-Tango | −0.658 | −1.310 | 0.117 | −0.221 | −0.081 | −0.645 | 0.000 | 0.253 |
| CCR8-Tango | −0.700 | −1.652 | −1.097 | −0.502 | −0.522 | −0.178 | −0.688 | −0.569 |
| CCRL2-Tango | −0.782 | −1.802 | 0.212 | 0.383 | −0.212 | −0.014 | 0.334 | 0.258 |
| CD97-Tango | −0.329 | −0.985 | −0.193 | 0.160 | −0.788 | −0.235 | 0.740 | −0.381 |
| CHRM1-Tango | 0.122 | −0.046 | −0.136 | 0.510 | −0.558 | −0.221 | −1.100 | −0.204 |
| CHRM2-Tango | 0.608 | 0.082 | 0.433 | 0.965 | −0.567 | −0.337 | −0.993 | 0.042 |
| CHRM3-Tango | 1.038 | −0.176 | 0.760 | 0.600 | −0.945 | −0.197 | −0.266 | 0.213 |
| CHRM4-Tango | 1.248 | −0.238 | −0.244 | 0.096 | −0.450 | −0.136 | −0.234 | 0.528 |
| CHRM5-Tango | 0.085 | −0.500 | 0.095 | 0.037 | −0.403 | −0.314 | −0.431 | 0.905 |
| CMKLR1-Tango | −0.180 | −0.555 | −0.217 | −0.022 | −0.445 | −0.246 | −0.605 | 0.255 |
| CNR1-Tango | −0.792 | −1.064 | −0.402 | 0.287 | −0.258 | −0.452 | −0.682 | 0.060 |
| CNR2-Tango | −0.537 | −0.852 | −0.104 | 0.082 | −0.282 | −0.544 | −0.369 | −0.353 |
| CRHR1-Tango | −0.229 | −0.904 | −0.373 | −0.202 | −1.119 | −0.434 | 0.170 | −0.171 |
| CRHR2-Tango | −0.211 | −0.702 | −1.086 | −0.341 | −0.054 | 0.000 | −0.844 | 0.300 |
| CX3C1-Tango | −0.328 | −0.791 | −0.335 | −0.621 | −0.720 | −0.985 | −0.358 | −0.070 |
| CXCR1-Tango | −0.238 | −1.257 | −0.675 | −0.033 | −0.031 | −0.101 | 0.165 | 0.288 |
| CXCR2-Tango | −0.809 | −0.980 | −0.589 | −0.944 | −1.148 | 0.142 | −0.866 | 0.027 |
| CXCR3-Tango | −1.007 | −1.098 | −0.355 | −0.148 | −0.538 | −0.144 | −0.500 | −0.190 |
| CXCR4-Tango | −0.610 | −0.557 | 0.121 | −0.084 | −0.421 | 0.461 | −0.543 | 0.073 |
| CXCR5-Tango | −0.309 | −0.754 | 0.124 | −0.096 | −0.588 | 0.447 | −0.039 | −0.229 |
| CXCR6-Tango | −0.665 | −1.193 | −1.007 | −0.200 | −0.079 | −0.340 | −0.046 | 0.547 |
| CXCR7-Tango | −1.517 | −1.731 | −0.778 | −0.739 | −0.478 | −0.352 | −0.316 | −0.540 |
| CYSLTR1-Tango | −0.527 | −0.899 | −1.050 | −0.564 | −0.342 | −0.433 | −1.078 | −0.134 |
| CYSLTR2-Tango | −0.553 | −1.213 | −0.364 | −0.253 | −0.344 | −0.188 | 0.000 | 0.284 |
| DRD1-Tango | −1.196 | −1.518 | −0.320 | −0.417 | −0.382 | −0.582 | 0.308 | 0.295 |
| DRD2-Tango | −1.035 | −1.605 | −0.588 | −0.759 | −0.844 | −0.436 | 3.107 | 0.730 |
| DRD3-Tango | −2.036 | −1.685 | −0.775 | 0.079 | −0.842 | −0.106 | 2.972 | 0.537 |
| DRD4-Tango | −2.449 | −2.449 | −0.463 | −0.026 | −0.969 | −0.047 | 2.135 | 0.497 |
| DRD5-Tango | −0.576 | −0.861 | −0.144 | −1.026 | −0.940 | −0.771 | −0.604 | −0.320 |
| EDNRA-Tango | −0.405 | −0.382 | −0.222 | −0.050 | −0.437 | −0.440 | −0.632 | 0.117 |
| EDNRB-Tango | −0.470 | −1.004 | 0.157 | 0.130 | −0.617 | −0.556 | −0.064 | −0.154 |
| ELTD1-Tango | −1.040 | −0.813 | −0.132 | −0.738 | −0.741 | −0.764 | −0.484 | −0.073 |
| F2R-Tango | −0.216 | −0.281 | −0.207 | −0.044 | −0.357 | −0.728 | −0.092 | 0.343 |
| F2RL1-Tango | −0.348 | −0.494 | −0.290 | 0.146 | −0.179 | −0.622 | −0.405 | 0.675 |
| F2RL2-Tango | −0.693 | −0.447 | 0.092 | −0.046 | −0.379 | −0.094 | −0.585 | 0.449 |
| F2RL3-Tango | −0.361 | −0.484 | −0.312 | −0.025 | −0.311 | −0.667 | −0.474 | 0.286 |
| FFA1-Tango | −0.655 | −0.736 | 0.306 | −0.078 | −0.528 | −0.530 | −1.036 | 0.138 |
| FFA2-Tango | −0.848 | −1.125 | 0.141 | 0.040 | −0.443 | −0.264 | −0.188 | 0.016 |
| FFA3-Tango | −0.722 | −0.843 | −0.258 | 0.090 | −0.099 | −0.208 | 0.253 | 0.433 |
| FPR1-Tango | −0.213 | −0.221 | −0.449 | 0.385 | 0.323 | −0.313 | −0.567 | −1.225 |
| FPR2-Tango | −0.698 | −0.990 | −0.123 | 0.359 | −0.397 | −0.339 | 0.261 | 0.225 |
| FPR3-Tango | −0.699 | −0.676 | −0.278 | −0.075 | −0.645 | −0.503 | −1.149 | −0.027 |
| FSHR-Tango | −0.573 | −0.731 | −0.138 | −0.271 | −0.373 | −0.448 | −0.430 | 0.209 |
| GABBR1-Tango | −0.252 | −0.897 | 0.090 | 0.094 | −0.266 | −0.225 | −0.051 | −0.091 |
| GAL1-Tango | −1.196 | −1.940 | −1.174 | −0.448 | −1.262 | −1.112 | −0.526 | −0.564 |
| GAL2-Tango | −0.234 | −1.390 | −0.298 | −0.027 | 0.629 | −0.181 | −0.554 | 0.369 |
| GAL3-Tango | −1.043 | −1.165 | −1.198 | 0.027 | 0.156 | −0.771 | −0.700 | −0.501 |
| GCGR-Tango | −0.777 | −0.955 | −0.500 | −0.361 | −0.275 | −0.528 | −0.642 | 0.072 |
| GHRHR-Tango | −0.657 | −1.230 | −0.462 | −0.410 | −0.567 | −0.476 | −1.134 | 0.079 |
| GHSR-Tango | −0.922 | −0.869 | −0.215 | −0.814 | −0.300 | −0.429 | −1.503 | 0.006 |
| GIPR-Tango | −1.120 | −1.191 | −0.401 | −0.803 | −0.461 | −1.021 | −0.829 | 0.430 |
| GLP1R-Tango | −0.503 | −0.303 | −0.451 | −0.281 | 0.068 | −0.188 | −0.995 | 0.185 |
| GLP2R-Tango | −0.520 | −0.734 | −0.782 | −0.234 | −0.193 | 0.147 | −1.647 | 0.152 |
| GNRHR-Tango | −0.570 | −1.570 | 0.112 | 0.089 | −0.414 | −0.226 | −0.458 | 0.085 |
| GPBA-Tango | −0.615 | −0.081 | −0.549 | 0.235 | −0.356 | −0.071 | −1.194 | −0.449 |
| GPER-Tango | −0.435 | −1.339 | −0.365 | −0.369 | −0.862 | −0.734 | −0.171 | 0.335 |
| GPR1-Tango | −0.990 | −1.334 | −0.426 | −0.556 | −0.589 | −0.397 | −0.788 | 0.144 |
| GPR101-Tango | −0.603 | −0.957 | −0.534 | 0.023 | −0.339 | −0.429 | −1.332 | 0.224 |
| GPR110-Tango | −0.389 | −0.599 | −0.274 | −0.049 | −0.275 | −0.714 | −0.600 | 0.256 |
| GPR111-Tango | −0.555 | −1.020 | −0.402 | −0.185 | −0.648 | −0.689 | −0.252 | −0.059 |
| GPR113-Tango | −0.978 | −0.748 | −0.207 | −0.631 | −0.377 | −0.213 | −0.484 | −0.135 |
| GPR114-Tango | −0.384 | −0.627 | −0.298 | −0.290 | −0.492 | −0.227 | −0.464 | −0.071 |
| GPR115-Tango | −0.686 | −0.737 | −0.322 | −0.381 | −0.351 | −0.003 | −0.746 | −0.204 |
| GPR116-Tango | −0.438 | −0.772 | −0.345 | −0.377 | −0.333 | −0.255 | −0.380 | 0.061 |
| GPR119-Tango | −0.650 | −1.041 | −0.236 | 0.056 | −0.521 | 0.026 | −0.116 | −0.073 |
| GPR12-Tango | −0.269 | −0.768 | −0.518 | −0.214 | −0.303 | −0.039 | −0.512 | 0.278 |
| GPR120-Tango | −0.904 | −0.556 | 0.247 | −0.011 | −0.721 | −0.204 | −0.387 | −0.412 |
| GPR123-Tango | −0.286 | −0.504 | −0.485 | 0.145 | −0.027 | −0.134 | −0.872 | 0.298 |
| GPR124-Tango | −0.708 | −1.064 | −0.259 | 0.093 | −0.596 | −0.101 | −0.484 | −0.005 |
| GPR125-Tango | −0.639 | −0.999 | −0.561 | −0.082 | −0.376 | −0.053 | −0.861 | 0.284 |
| GPR126-Tango | −0.927 | −1.471 | −0.166 | −0.158 | −0.490 | −0.420 | −0.537 | −0.090 |
| GPR132-Tango | −0.847 | −0.989 | 0.000 | 0.366 | 0.385 | −0.201 | −1.274 | −0.268 |
| GPR133-Tango | −0.853 | −1.139 | −0.011 | −0.003 | 0.097 | −0.179 | −0.803 | −0.120 |
| GPR135-Tango | −0.816 | −1.198 | −0.440 | −0.141 | −0.018 | −0.423 | −0.694 | −0.150 |
| GPR141-Tango | −0.989 | −0.751 | −0.023 | 0.054 | −0.333 | −0.269 | −0.031 | −0.640 |
| GPR142-Tango | −0.760 | −1.088 | −0.296 | 0.348 | −0.092 | −0.256 | −1.040 | −0.297 |
| GPR143-Tango | −1.044 | −0.724 | −0.299 | 0.103 | −0.131 | −0.334 | −0.931 | 0.658 |
| GPR144-Tango | −0.423 | −0.858 | −0.110 | −0.227 | −0.843 | −0.474 | −0.515 | −0.547 |
| GPR146-Tango | −0.317 | −0.243 | −0.066 | −0.263 | −0.688 | −0.812 | −0.404 | 0.263 |
| GPR148-Tango | −0.066 | −0.066 | −0.170 | 1.186 | −0.483 | 0.216 | −0.404 | 0.697 |
| GPR149-Tango | −1.135 | −1.052 | −0.033 | 0.244 | −0.509 | 0.017 | −1.033 | 0.541 |
| GPR15-Tango | −0.594 | −0.955 | −0.613 | 0.025 | −0.493 | −0.144 | 0.000 | 0.832 |
| GPR150-Tango | −0.525 | −0.866 | −0.160 | 0.245 | −0.788 | −0.800 | −0.160 | 0.282 |
| GPR151-Tango | −0.294 | −0.726 | −0.009 | 0.701 | −0.295 | −0.196 | −0.538 | 0.591 |
| GPR152-Tango | −0.485 | −0.517 | −0.281 | 0.170 | −0.262 | −0.293 | −0.245 | −0.442 |
| GPR153-Tango | −0.534 | −0.595 | −0.598 | −0.021 | −0.249 | −0.023 | −0.788 | 0.126 |
| GPR156-Tango | −0.662 | −0.752 | −0.858 | −0.065 | −0.568 | 0.086 | −0.764 | −0.513 |
| GPR157-Tango | −0.327 | −0.937 | −0.101 | 0.253 | −0.271 | −0.213 | −0.215 | 0.407 |
| GPR158-Tango | −0.114 | −0.405 | −0.432 | 0.431 | 0.029 | −0.128 | −0.399 | −0.051 |
| GPR160-Tango | −0.375 | −0.777 | −0.310 | 0.557 | 0.466 | −0.056 | −0.429 | 0.458 |
| GPR161-Tango | −0.747 | −0.899 | −0.162 | 0.189 | 0.009 | −0.207 | −0.391 | 0.704 |
| GPR162-Tango | −0.903 | −1.255 | −0.266 | −0.240 | −0.386 | −0.544 | −0.442 | −0.042 |
| GPR17-Tango | −1.140 | −0.881 | −0.120 | 0.062 | −0.759 | 0.075 | −0.565 | 0.378 |
| GPR171-Tango | −1.126 | −1.220 | 0.058 | 0.424 | −0.314 | 0.255 | −0.146 | 0.069 |
| GPR173-Tango | −0.943 | −1.049 | −0.366 | 0.625 | −0.436 | −0.422 | −0.386 | 0.084 |
| GPR174-Tango | −0.575 | −1.012 | 0.668 | 0.448 | 0.108 | −0.071 | 0.351 | −0.300 |
| GPR18-Tango | −0.820 | −1.251 | 0.062 | 0.304 | 0.078 | −0.341 | 0.379 | 0.270 |
| GPR182-Tango | −1.006 | −1.354 | −0.099 | 0.254 | −0.293 | −0.204 | −0.730 | 0.318 |
| GPR183-Tango | −0.730 | −1.323 | 0.035 | 0.339 | −0.424 | −0.276 | −0.006 | 0.020 |
| GPR19-Tango | −0.817 | −0.612 | 0.054 | 0.476 | −0.430 | −0.124 | −0.208 | 0.601 |
| GPR20-Tango | −0.241 | −1.082 | 1.372 | 0.193 | −0.205 | 0.000 | 0.497 | 0.511 |
| GPR21-Tango | 0.064 | −1.000 | 0.055 | 0.135 | −0.117 | 0.016 | 0.200 | 0.387 |
| GPR22-Tango | −0.485 | −0.672 | −0.318 | 0.280 | −0.007 | −0.035 | −0.033 | 0.556 |
| GPR25-Tango | −0.362 | −0.988 | 0.144 | 0.023 | −0.389 | −0.060 | 0.275 | −0.129 |
| GPR26-Tango | −0.690 | −0.769 | −0.164 | 0.083 | −0.390 | −0.420 | −0.243 | 0.720 |
| GPR27-Tango | 0.126 | −0.347 | 0.053 | −0.109 | −0.628 | 0.039 | 0.111 | 0.047 |
| GPR3-Tango | −1.107 | −1.304 | 0.022 | 0.124 | −0.669 | −0.467 | −0.278 | −0.345 |
| GPR31-Tango | −0.931 | −1.429 | 0.354 | 0.041 | −0.542 | −0.365 | 0.331 | −0.012 |
| GPR32-Tango | −0.648 | −0.911 | −0.371 | −0.510 | −0.866 | −0.232 | −1.299 | −0.180 |
| GPR34-Tango | −0.820 | −1.659 | −0.189 | −0.426 | −0.925 | −0.333 | −0.356 | −0.264 |
| GPR35-Tango | −1.019 | −1.516 | −0.449 | −0.098 | −0.433 | −0.140 | −1.073 | −0.290 |
| GPR37-Tango | −0.255 | −0.835 | −0.148 | −0.258 | −0.631 | 0.066 | −1.033 | 0.022 |
| GPR37L1-Tango | −0.359 | −0.763 | −0.384 | −0.264 | −0.378 | 0.248 | −0.886 | 0.401 |
| GPR39-Tango | −0.540 | −0.902 | −0.421 | −0.399 | −0.577 | −0.285 | −0.773 | −0.321 |
| GPR4-Tango | −0.884 | −0.759 | 0.202 | −0.224 | −0.321 | −0.019 | −1.165 | 0.056 |
| GPR44-Tango | −0.968 | −1.126 | −0.110 | −0.439 | −0.404 | −0.511 | −0.714 | −0.205 |
| GPR45-Tango | −0.444 | −0.409 | −0.038 | −1.284 | −0.518 | −0.399 | −0.273 | 0.374 |
| GPR50-Tango | 0.125 | −0.685 | −0.050 | 0.402 | −0.301 | −0.064 | −0.120 | 0.798 |
| GPR52-Tango | −0.974 | −0.849 | −0.436 | −0.236 | −0.308 | −0.189 | −0.497 | 0.611 |
| GPR55-Tango | −0.371 | −0.437 | −0.202 | −0.049 | −0.436 | −0.098 | 0.392 | 0.540 |
| GPR56-Tango | −0.579 | −0.606 | 0.574 | −0.061 | −0.596 | −0.010 | 0.905 | 0.507 |
| GPR6-Tango | −0.386 | −0.712 | 0.122 | −0.197 | −0.145 | −0.033 | −0.252 | 0.071 |
| GPR61-Tango | −0.375 | −0.285 | 0.008 | −0.138 | −0.078 | 0.000 | −0.557 | 0.380 |
| GPR62-Tango | −0.324 | −0.423 | 0.353 | 0.172 | −0.415 | −0.180 | 0.170 | 0.473 |
| GPR63-Tango | −0.862 | −0.696 | −0.644 | −0.141 | −0.157 | −0.071 | −0.740 | −0.290 |
| GPR64-Tango | −0.640 | −0.798 | −0.625 | −0.382 | −0.230 | −0.259 | −0.907 | −0.102 |
| GPR65-Tango | −0.370 | −0.296 | −0.401 | −0.138 | −0.180 | −0.135 | −0.814 | 0.087 |
| GPR68-Tango | −0.350 | −0.356 | −0.183 | −0.291 | −0.481 | −0.326 | −1.241 | −0.253 |
| GPR75-Tango | −0.090 | −0.406 | −0.280 | −0.378 | −0.308 | −0.185 | −0.420 | 0.446 |
| GPR77-Tango | −0.656 | −0.830 | −0.745 | −0.896 | −0.725 | −0.169 | −0.927 | −0.101 |
| GPR78-Tango | −0.357 | −0.506 | −0.082 | 0.186 | −0.185 | 0.022 | −0.037 | 0.377 |
| GPR82-Tango | −0.784 | −0.826 | −0.672 | −0.638 | −0.452 | −0.104 | −0.689 | −0.039 |
| GPR83-Tango | −0.133 | −0.361 | −0.344 | −0.172 | −0.277 | −0.013 | −0.570 | 0.037 |
| GPR84-Tango | −0.087 | 0.085 | −0.308 | −0.480 | −0.362 | 0.005 | −0.297 | 0.132 |
| GPR85-Tango | −0.116 | −0.563 | 0.030 | −0.173 | −0.329 | 0.135 | −0.121 | −0.246 |
| GPR87-Tango | −0.207 | −0.237 | −0.417 | −0.307 | −0.340 | 0.191 | −0.655 | −0.088 |
| GPR88-Tango | −0.107 | −0.631 | −0.192 | 0.000 | −0.550 | −0.071 | −0.316 | −0.081 |
| GPR97-Tango | −0.390 | −0.674 | −0.139 | −0.234 | −0.242 | −0.026 | −0.597 | 0.000 |
| GPRC5A-Tango | −0.335 | −0.149 | −0.041 | −0.485 | −0.182 | −0.043 | −0.175 | 0.125 |
| GPRC5B-Tango | −0.260 | −0.330 | −0.181 | −0.245 | −0.131 | −0.035 | 0.200 | 0.435 |
| GPRC5C-Tango | 0.114 | 0.000 | −0.400 | −0.289 | 0.088 | −0.095 | −0.144 | 0.482 |
| GPRC5D-Tango | −0.309 | −0.399 | −0.476 | −0.122 | −0.314 | 0.027 | −0.946 | −0.209 |
| GPRC6A-Tango | 0.031 | −0.081 | −0.428 | −0.306 | −0.428 | 0.428 | −0.575 | −0.498 |
| GRM1-Tango | −0.198 | −0.173 | −0.198 | −0.508 | −0.336 | 0.283 | −0.690 | 0.358 |
| GRM2-Tango | −0.324 | −0.547 | −0.369 | −0.343 | −0.403 | −0.139 | −0.228 | −0.035 |
| GRM4-Tango | 0.007 | 0.153 | −0.048 | −0.152 | 0.538 | −0.087 | −0.284 | 0.665 |
| GRM5-Tango | −0.080 | −0.416 | −0.618 | 0.306 | 0.251 | −0.236 | −1.275 | 0.585 |
| GRM6-Tango | −0.043 | −0.383 | −0.997 | −0.493 | −0.704 | −0.050 | −0.891 | −0.039 |
| GRM7-Tango | −0.199 | −0.175 | −0.757 | −0.652 | −0.557 | −0.553 | −1.115 | −0.685 |
| GRM8-Tango | −0.394 | −0.691 | −0.216 | −0.028 | −0.495 | −0.411 | −0.082 | 0.372 |
| GRPR-Tango | 0.148 | −0.231 | −0.318 | −0.095 | 0.141 | −0.270 | −0.134 | 0.545 |
| HCA1-Tango | −0.250 | −0.610 | 0.118 | −0.195 | 0.158 | 0.094 | 0.258 | 0.112 |
| HCA2-Tango | 0.110 | 0.199 | 0.108 | 0.036 | −0.095 | −0.077 | −0.448 | 0.565 |
| HCA3-Tango | −0.481 | −1.216 | 0.356 | 0.072 | −0.200 | −0.218 | 0.215 | −0.402 |
| HCTR1-Tango | −0.664 | −0.781 | 0.313 | −0.089 | −0.508 | 0.000 | 0.801 | 0.377 |
| HCTR2-Tango | −0.719 | −0.719 | −1.132 | 0.480 | −0.382 | −0.234 | −0.341 | 0.376 |
| HRH1-Tango | −0.480 | −0.604 | −0.434 | −0.076 | −0.808 | −0.271 | −0.952 | 0.235 |
| HRH2-Tango | 0.703 | 0.075 | 0.212 | 0.233 | −0.601 | 0.128 | 0.057 | −0.056 |
| HRH3-Tango | −0.572 | −0.011 | −0.913 | 0.197 | −0.605 | 0.160 | −0.781 | 0.056 |
| HRH4-Tango | −0.940 | −0.898 | −0.462 | 0.184 | −0.560 | −0.241 | 0.017 | 0.325 |
| HTR1A-Tango | −0.454 | −0.676 | −0.029 | 0.198 | −0.562 | 0.198 | 1.566 | 0.124 |
| HTR1B-Tango | −0.673 | −0.687 | −0.373 | −0.132 | −0.746 | −0.306 | −0.136 | 0.196 |
| HTR1D-Tango | −0.797 | −1.029 | −0.412 | −0.043 | −0.473 | −0.416 | 0.176 | −0.168 |
| HTR1E-Tango | −0.741 | −0.785 | −0.107 | −0.088 | −0.476 | −0.062 | −0.381 | 0.259 |
| HTR1F-Tango | −0.393 | −0.502 | 0.199 | −0.365 | 0.139 | −0.287 | 0.131 | −0.009 |
| HTR2A-Tango | −0.547 | −0.788 | −0.089 | −0.141 | −0.285 | −0.605 | −0.634 | 0.072 |
| HTR2B-Tango | −1.637 | −0.419 | 0.192 | −0.162 | −0.308 | −0.165 | −0.748 | 0.111 |
| HTR2C-Tango | −1.285 | −1.120 | −0.087 | −0.066 | −0.555 | −0.389 | −0.156 | 0.052 |
| HTR4-Tango | −1.990 | −1.780 | −0.056 | −0.703 | −0.327 | −0.589 | 0.829 | −0.055 |
| HTR5-Tango | −1.263 | −1.224 | −0.095 | −0.495 | 0.621 | −0.504 | −0.076 | 0.246 |
| HTR6-Tango | −1.238 | −1.703 | 0.120 | 0.215 | 0.206 | −0.545 | 0.826 | 0.093 |
| HTR7-Tango | −2.771 | −2.662 | 0.124 | 0.281 | 1.407 | −0.615 | −0.139 | 0.744 |
| KISSPEPTIN-R- | −0.022 | 0.097 | −0.184 | 0.123 | −0.804 | −0.492 | −0.184 | 0.054 |
| Tango | | | | | | | | |
| LHCGR-Tango | −0.111 | 0.199 | −0.015 | −0.016 | −0.294 | −0.285 | −0.130 | 0.228 |
| LPAR1-Tango | −0.107 | −0.186 | −0.285 | 0.039 | −0.312 | −0.368 | −0.111 | 0.141 |
| LPAR2-Tango | −0.370 | −0.933 | −0.411 | −0.053 | −0.284 | −0.431 | −0.010 | −0.119 |
| LPAR4-Tango | −0.338 | −0.202 | −0.470 | −0.017 | −0.352 | −0.636 | −0.796 | 0.096 |
| LPAR5-Tango | −0.443 | −0.770 | −0.079 | −0.136 | −0.301 | −0.409 | −0.390 | 0.166 |
| LPAR6-Tango | 0.155 | 0.123 | −0.136 | 0.349 | −0.100 | −0.420 | 0.288 | 0.636 |
| LTB4R-Tango | −0.202 | −0.202 | −0.014 | −0.032 | −0.440 | −0.453 | −0.042 | 0.128 |
| LTB4R2B-Tango | −0.259 | −0.066 | −0.046 | −0.023 | −0.257 | −0.393 | 0.040 | 0.417 |
| MAS1-Tango | −0.327 | −0.615 | −0.265 | −0.208 | −0.778 | −0.102 | −0.678 | −0.065 |
| MAS1L-Tango | −0.158 | 0.067 | −0.382 | −0.168 | −0.338 | −0.242 | −0.933 | 0.189 |
| MC1R-Tango | −0.202 | −0.517 | −0.055 | 0.013 | −0.569 | −0.099 | −0.527 | 0.173 |
| MC2R-Tango | −0.449 | −0.635 | −0.128 | −0.166 | −0.675 | −0.166 | −0.050 | 0.533 |
| MC3R-Tango | −0.530 | −0.761 | −0.858 | 0.161 | 0.088 | −0.517 | 0.266 | 1.051 |
| MC4R-Tango | −0.299 | −0.909 | 0.062 | −0.558 | −0.679 | 0.243 | 0.542 | 0.429 |
| MC5R-Tango | −0.344 | −0.510 | −0.365 | −0.178 | −0.498 | −0.528 | −0.187 | 0.289 |
| MCHR1-Tango | −0.420 | −0.670 | −1.177 | −0.486 | −0.206 | 0.158 | −1.419 | 0.633 |
| MCHR2-Tango | −0.064 | −0.305 | −0.309 | 0.032 | −0.104 | −0.055 | −0.501 | 0.469 |
| MLNR-Tango | −0.246 | −0.952 | −0.090 | 0.097 | −0.511 | 0.242 | −0.661 | −0.011 |
| MRGPRD-Tango | −0.498 | −0.955 | −0.316 | −0.333 | −0.462 | 0.102 | −0.715 | −0.088 |
| MRGPRE-Tango | −0.830 | −0.933 | −0.307 | −0.193 | −0.384 | 0.099 | −0.396 | −0.064 |
| MRGPRF-Tango | −0.086 | −0.311 | −0.402 | −0.488 | −0.181 | 0.035 | −0.192 | −0.094 |
| MRGPRG-Tango | −0.867 | −0.756 | −1.096 | −0.441 | 0.320 | 0.116 | −1.058 | 0.095 |
| MRGPRX1-Tango | −1.459 | −1.340 | −0.919 | −0.440 | 0.142 | 0.050 | −0.626 | 0.870 |
| MRGPRX2-Tango | −1.029 | −0.888 | −0.759 | −0.540 | −0.678 | 0.118 | −0.366 | 0.311 |
| MRGPRX3-Tango | −0.822 | −1.276 | 0.181 | −0.494 | −0.820 | −0.038 | −0.357 | −0.019 |
| MRGPRX4-Tango | −1.331 | −1.196 | −0.787 | −1.045 | −0.533 | 0.191 | −1.239 | −0.332 |
| MTNR1A-Tango | −0.555 | −0.581 | −1.342 | −0.840 | −0.865 | 0.446 | −0.547 | 0.044 |
| MTNR1B-Tango | −0.377 | −1.122 | −1.266 | −0.796 | −0.792 | 0.107 | 0.112 | 0.236 |
| NMBR-Tango | −0.248 | −0.451 | −1.113 | −0.202 | 0.065 | 0.060 | −0.596 | 0.600 |
| NMUR1-Tango | −0.526 | −0.876 | −0.117 | −0.627 | −0.786 | −0.214 | 0.193 | −0.482 |
| NMUR2-Tango | −0.045 | −0.877 | 0.122 | −0.214 | −0.013 | 0.237 | 0.807 | −0.160 |
| NPBW1-Tango | −0.848 | −1.350 | 0.533 | 0.234 | −0.153 | 0.177 | 0.240 | 0.260 |
| NPBW2-Tango | −1.135 | −1.069 | −0.321 | −0.511 | −0.404 | −0.011 | −0.198 | −0.302 |
| NPFF1-Tango | −0.387 | −0.695 | −1.191 | −0.108 | −0.340 | 0.336 | −1.693 | −0.228 |
| NPFF2-Tango | 0.072 | −0.473 | −0.341 | 0.404 | 0.459 | 0.205 | −0.177 | 0.082 |
| NPS-Tango | −1.020 | −1.278 | −1.123 | −0.105 | −0.045 | −0.307 | −0.478 | 0.638 |
| NPY1R-Tango | −0.920 | −1.039 | −0.757 | 0.336 | 0.155 | −0.072 | −0.807 | 0.544 |
| NPY2R-Tango | −0.986 | −0.390 | −0.300 | 0.197 | 0.337 | −0.020 | −0.300 | 0.430 |
| NPY4R-Tango | −1.051 | −1.316 | −0.034 | −0.214 | −0.177 | −0.250 | −0.080 | 0.049 |
| NPY5R-Tango | −0.309 | −0.896 | −0.404 | −0.288 | −0.334 | 0.275 | −0.318 | −0.162 |
| NTSR1-Tango | −0.419 | −0.593 | −0.122 | −0.282 | −0.517 | −0.295 | −0.861 | −0.829 |
| NTSR2-Tango | 0.152 | −0.713 | 0.348 | 0.136 | −0.478 | −0.438 | 0.013 | −0.561 |
| OPN3-Tango | −0.131 | −0.758 | −0.243 | −0.421 | −0.614 | −0.044 | −0.243 | 0.212 |
| OPN5-Tango | −0.650 | −0.851 | −0.378 | −0.657 | −0.659 | −0.084 | −0.560 | −0.177 |
| OPRD1-Tango | −0.757 | −1.503 | −0.511 | −0.350 | −0.627 | −0.189 | −0.856 | −0.374 |
| OPRK1-Tango | 0.176 | −0.232 | −0.213 | 0.289 | −0.151 | −0.276 | −0.342 | 0.227 |
| OPRL1-Tango | −0.188 | −0.834 | −0.638 | 0.357 | −0.304 | −0.579 | −0.873 | 0.317 |
| OPRM1-Tango | 0.516 | −0.058 | −0.231 | −0.150 | −0.399 | −0.473 | −0.588 | 0.016 |
| OXER1-Tango | 0.428 | 0.166 | −0.429 | −0.005 | 0.015 | −0.117 | −0.743 | 0.193 |
| OXGR1-Tango | −0.882 | −1.212 | −1.089 | −0.502 | −0.772 | −0.170 | −1.340 | −0.480 |
| OXTR-Tango | −0.078 | −0.952 | 0.131 | −0.176 | −0.677 | −0.361 | 0.131 | −0.094 |
| P2RY1-Tango | −0.458 | −0.277 | 0.104 | 0.635 | −0.198 | −0.118 | −0.705 | −1.073 |
| P2RY10-Tango | −0.186 | −0.650 | −0.085 | 0.124 | −0.236 | −0.174 | −0.176 | −0.758 |
| P2RY11-Tango | −0.628 | −0.655 | 0.335 | −0.238 | −0.343 | −0.580 | −0.153 | −0.621 |
| P2RY12-Tango | −0.733 | −0.552 | −0.666 | −0.547 | −0.299 | −0.269 | 0.413 | −1.411 |
| P2RY13-Tango | −0.852 | −0.825 | −0.319 | −0.089 | −0.996 | −0.481 | −0.848 | −0.287 |
| P2RY14-Tango | −1.112 | −0.897 | −0.384 | −0.250 | −0.826 | −0.205 | −0.597 | −0.184 |
| P2RY2-Tango | −0.680 | −0.998 | −0.060 | 0.101 | −0.813 | −0.061 | −0.463 | −0.529 |
| P2RY4-Tango | −1.406 | −1.603 | −0.165 | −0.220 | −0.473 | −0.117 | −0.802 | −0.511 |
| P2RY6-Tango | −1.316 | −1.268 | −0.301 | −0.148 | −0.994 | 0.122 | −0.844 | −0.360 |
| P2RY8-Tango | −1.154 | −1.416 | −0.617 | −0.259 | −0.296 | −0.039 | −0.770 | −0.228 |
| PK1-Tango | −0.882 | −1.564 | −0.462 | −0.096 | −0.712 | −0.337 | −0.482 | 0.087 |
| PK2-Tango | −0.128 | −0.619 | −0.100 | 0.021 | −0.582 | 0.041 | −0.144 | 0.095 |
| PRRP-Tango | −0.794 | −1.168 | 0.014 | −0.076 | −0.849 | −0.398 | −0.154 | −0.217 |
| PTAFR-Tango | −0.714 | −0.764 | 0.072 | −0.057 | −0.396 | −0.177 | −0.660 | −0.549 |
| PTGDR-Tango | −0.484 | −0.978 | 0.301 | −0.411 | −0.376 | −0.325 | −0.139 | −0.260 |
| PTGER1-Tango | −0.234 | −0.752 | 0.327 | 0.094 | −0.457 | −0.230 | −0.036 | 0.322 |
| PTGER2-Tango | −0.156 | −1.034 | 0.494 | −0.146 | −0.405 | −0.218 | 0.292 | −0.108 |
| PTGER3-Tango | −0.893 | −0.736 | −0.064 | −0.142 | −0.360 | 0.119 | 0.058 | 0.359 |
| PTGER4-Tango | −0.863 | −0.921 | −0.063 | 0.198 | 0.185 | 0.062 | 0.646 | 1.125 |
| PTGFR-Tango | −0.447 | −1.136 | 0.098 | −0.147 | −0.532 | −0.693 | 0.305 | 0.646 |
| PTGIR-Tango | −0.627 | −1.300 | 0.528 | −0.113 | −0.495 | −0.231 | 0.143 | 0.048 |
| PTH1R-Tango | −0.423 | −1.045 | −0.265 | −0.038 | −0.489 | 0.088 | −0.265 | 0.141 |
| PTH2R-Tango | −0.893 | −1.267 | −0.380 | −0.320 | −0.190 | 0.126 | −0.562 | −0.134 |
| QRFP-Tango | −0.328 | −0.700 | 0.118 | −0.364 | −0.378 | −0.179 | −0.154 | 0.013 |
| RXFP1-Tango | −0.690 | −1.002 | −0.168 | −0.341 | −0.071 | −0.842 | −0.264 | 0.000 |
| RXFP2-Tango | −0.712 | −0.573 | −0.217 | −0.171 | −0.291 | −0.186 | −0.406 | 0.037 |
| RXFP3-Tango | −0.498 | −0.890 | −0.293 | −0.773 | −0.342 | −0.278 | −0.699 | −0.105 |
| RXFP4-Tango | −0.845 | −0.790 | 0.082 | −0.795 | −0.370 | −0.555 | −0.213 | −0.209 |
| S1PR1-Tango | −0.437 | −1.345 | −0.173 | −0.675 | −0.268 | −0.137 | 0.081 | −0.499 |
| S1PR2-Tango | −0.521 | −1.161 | −0.515 | −0.482 | −0.378 | −0.390 | 0.055 | −0.387 |
| S1PR3-Tango | 0.112 | −0.139 | −0.027 | −0.234 | 0.038 | −0.161 | 0.154 | −0.138 |
| S1PR4-Tango | −0.945 | −1.105 | −0.628 | −1.169 | −0.854 | −0.054 | −0.984 | −0.464 |
| S1PR5-Tango | −0.825 | −1.240 | 0.198 | −0.137 | −0.624 | −0.353 | −0.087 | −0.493 |
| SCTR-Tango | −0.611 | −0.994 | −0.342 | −0.390 | −0.759 | −0.384 | 0.812 | 0.109 |
| SSTR1-Tango | −0.115 | −0.503 | −1.328 | −0.195 | −0.156 | 0.074 | −0.371 | 0.558 |
| SSTR2-Tango | −0.071 | −0.184 | −0.761 | −0.114 | −0.230 | −0.441 | 0.316 | 0.528 |
| SSTR3-Tango | −0.463 | −0.563 | 0.480 | 0.025 | −0.092 | 0.076 | 0.117 | 0.411 |
| SSTR4-Tango | −0.101 | −1.787 | −0.449 | −0.397 | −0.490 | −0.224 | −0.303 | 0.665 |
| SSTR5-Tango | −0.995 | −1.488 | −0.040 | 1.195 | −0.019 | 0.862 | 0.293 | 1.284 |
| SUCNR1-Tango | −1.625 | −1.803 | −0.404 | 0.530 | 0.569 | 0.329 | −0.153 | 0.685 |
| TA1-Tango | −0.494 | −1.006 | −0.198 | −0.058 | −0.204 | 0.272 | 0.043 | 0.401 |
| TAAR2-Tango | −0.296 | −0.796 | −0.366 | −0.141 | −0.212 | −0.240 | −0.419 | −0.129 |
| TAAR5-Tango | −0.584 | −1.044 | −0.948 | −0.342 | −0.719 | −0.073 | −1.123 | −0.443 |
| TAAR6-Tango | −0.531 | −0.430 | −0.639 | −0.555 | −0.327 | −0.037 | −0.749 | −0.074 |
| TAAR8-Tango | −0.086 | −0.276 | −1.050 | −0.315 | −0.048 | −0.022 | −1.005 | 0.152 |
| TAAR9-Tango | −0.276 | −0.475 | −1.039 | −0.091 | −0.014 | −0.425 | −0.588 | −0.124 |
| TACR1-Tango | −0.489 | −0.511 | −0.584 | −0.364 | −0.337 | −0.309 | −0.304 | −0.037 |
| TACR2-Tango | −0.433 | −1.278 | −0.886 | −0.385 | −0.594 | −0.393 | 0.132 | 0.077 |
| TACR3-Tango | −0.707 | −0.865 | −0.453 | −0.536 | −0.486 | −0.061 | 0.186 | 0.078 |
| TBXA2R-Tango | −0.535 | −1.284 | −0.222 | −0.411 | −0.279 | −0.203 | −0.153 | −0.107 |
| TSHR-Tango | −0.694 | −0.451 | −0.491 | −0.103 | −0.149 | −0.075 | −0.611 | 0.401 |
| UTS2R-Tango | −0.298 | −0.600 | −0.500 | 0.000 | −0.630 | −0.124 | −0.680 | 0.260 |
| VIPR1-Tango | −0.110 | −0.117 | −1.633 | −0.757 | −0.308 | −0.297 | −1.072 | 0.027 |
| VIPR2-Tango | 0.837 | 0.104 | −0.288 | 1.011 | −0.280 | 0.140 | 0.533 | 1.405 |

| | Acidamino- | Acidamino- | Acidamino- | | | Pepto- | Pepto- | |
|---|---|---|---|---|---|---|---|---|
| PRESTO-Tango | coccus | coccus | coccus | Megasphaera | Megasphaera | niphilus | niphilus | Bacteroids |
| with GMM | spp-1# | spp-2# | spp-3# | spp-1# | spp-2# | spp-1# | spp-2# | fragilis-1# |
| ADCYAP1R1- | −0.633 | −0.401 | −0.043 | −0.319 | −0.731 | −0.248 | −0.502 | −0.828 |
| Tango | | | | | | | | |
| ADORA1-Tango | −0.806 | −0.979 | −0.462 | −1.083 | −0.911 | −0.490 | −0.707 | −1.707 |
| ADORA2A-Tango | −0.083 | −0.636 | −0.298 | −0.306 | −0.169 | 0.135 | −0.379 | −1.065 |
| ADORA2B-Tango | −0.060 | −0.446 | −0.174 | −0.071 | −0.201 | −0.141 | −0.792 | −0.758 |
| ADORA3-Tango | 0.259 | −2.307 | 0.514 | 0.556 | 0.446 | −0.092 | −0.292 | −0.625 |
| ADRA1A-Tango | −0.333 | 0.101 | −0.167 | −0.192 | −0.009 | −0.174 | −0.215 | −0.934 |
| ADRA1B-Tango | −0.063 | −0.257 | 0.070 | −0.563 | 0.351 | −0.754 | −0.703 | −0.713 |
| ADRA1D-Tango | −0.140 | 0.033 | −0.028 | −0.719 | 0.346 | −0.292 | −0.134 | −0.899 |
| ADRA2A-Tango | −0.086 | −1.567 | −0.086 | 1.157 | 0.047 | 0.405 | −0.085 | −3.042 |
| ADRA2B-Tango | −0.812 | −0.678 | −0.671 | −0.194 | −0.715 | −0.125 | −0.089 | −2.048 |
| ADRA2C-Tango | 0.108 | −0.403 | 0.601 | −0.047 | −0.103 | 0.160 | 0.082 | −1.370 |
| ADRB1-Tango | −0.226 | −0.160 | 0.038 | 0.680 | 0.130 | 0.174 | −0.089 | −1.468 |
| ADRB2-Tango | −0.239 | 0.000 | −0.159 | 0.143 | −0.071 | −0.199 | −0.282 | −1.262 |
| ADRB3-Tango | −0.110 | −1.600 | 0.322 | 0.000 | 0.359 | −0.154 | −0.350 | −0.926 |
| AGTR1-Tango | 0.043 | −0.386 | 0.266 | 0.030 | 0.270 | 0.453 | 0.837 | −1.188 |
| AGTR2-Tango | −0.246 | 0.202 | −0.194 | 0.038 | −0.002 | −0.012 | 0.341 | −0.923 |
| APJ-Tango | −0.363 | 0.201 | 0.100 | −0.055 | 0.137 | 0.179 | −0.339 | −1.722 |
| AVPR1A-Tango | −0.188 | −0.982 | −0.233 | −0.025 | 0.151 | −0.255 | 0.000 | −1.511 |
| AVPR1B-Tango | 0.266 | −0.536 | 0.200 | −0.069 | 0.214 | −0.391 | −0.311 | −1.197 |
| AVPR2-Tango | −0.393 | −0.956 | −0.052 | −0.088 | −0.182 | 0.086 | −0.086 | −1.798 |
| BB3-Tango | −0.374 | −0.791 | −0.152 | −0.407 | −0.304 | −0.286 | −0.283 | −1.421 |
| BDKBR1-Tango | −0.128 | −0.978 | 0.135 | −0.176 | 0.217 | −0.020 | 0.107 | −0.788 |
| BDKBR2-Tango | 0.136 | 0.135 | 0.186 | 0.450 | 0.211 | −0.241 | −0.144 | −0.753 |
| C3AR1-Tango | −0.318 | −0.505 | −0.275 | −0.276 | −0.385 | −0.027 | 0.118 | −0.852 |
| C5A-Tango | 0.421 | 0.238 | 0.452 | 0.201 | 0.708 | −0.639 | −0.575 | −0.534 |
| CALCRb-Tango | 0.000 | −0.534 | 0.019 | 0.168 | −0.035 | −0.474 | −1.007 | −0.703 |
| CALCRL-Tango | 0.008 | −0.428 | −0.173 | 0.040 | 0.076 | −0.371 | −0.405 | −0.508 |
| CASR-Tango | −0.098 | −0.639 | 0.175 | −0.501 | −0.075 | −0.691 | −0.809 | −0.685 |
| CCKAR-Tango | −0.060 | −0.471 | 0.306 | −0.394 | 0.076 | −0.509 | −0.512 | −0.549 |
| CCKBR-Tango | −0.176 | −0.768 | −0.163 | −0.603 | −0.359 | −1.005 | −0.931 | −0.972 |
| CCR10-Tango | −0.412 | −0.829 | −0.363 | −0.459 | −0.176 | −0.612 | −0.806 | −1.185 |
| CCR2-Tango | −0.946 | −0.293 | −0.429 | 0.214 | −0.044 | −0.629 | −0.928 | −0.918 |
| CCR3-Tango | −1.078 | 0.096 | −0.391 | −0.449 | −0.537 | 0.183 | 0.385 | −1.934 |
| CCR4-Tango | −0.018 | 0.242 | −0.040 | 0.216 | −0.014 | −0.690 | −0.040 | −1.028 |
| CCR5-Tango | 0.574 | 0.102 | 0.401 | 0.204 | 0.605 | −0.170 | −0.243 | −0.746 |
| CCR6-Tango | 0.466 | 0.906 | 0.501 | 0.647 | 0.860 | 0.108 | 0.209 | −0.240 |
| CCR7-Tango | −0.276 | −0.595 | −0.210 | 0.029 | −0.017 | −0.520 | −0.565 | −0.903 |
| CCR8-Tango | −0.445 | −0.429 | −0.085 | 0.223 | 0.399 | 0.405 | −0.129 | −1.238 |
| CCRL2-Tango | 0.640 | −0.257 | 0.447 | 0.674 | 0.640 | 0.044 | 0.672 | −1.017 |
| CD97-Tango | −0.218 | −0.342 | −0.020 | 0.049 | 0.284 | −0.483 | −0.756 | −0.986 |
| CHRM1-Tango | 0.168 | −1.445 | 0.102 | −0.075 | 0.348 | 0.136 | 0.026 | −0.566 |
| CHRM2-Tango | −0.197 | −0.894 | 0.052 | −0.204 | −0.192 | −0.410 | −0.643 | −0.936 |
| CHRM3-Tango | −0.443 | −0.598 | −0.241 | 0.233 | −0.147 | 0.181 | −0.034 | −1.139 |
| CHRM4-Tango | −0.150 | −0.969 | −0.047 | 0.081 | −0.036 | 0.490 | 0.331 | −0.339 |
| CHRM5-Tango | 0.031 | 0.116 | 0.217 | 0.141 | −0.112 | −0.594 | −0.803 | −0.687 |
| CMKLR1-Tango | −0.091 | −0.205 | −0.130 | 0.013 | 0.117 | −0.803 | −0.296 | −0.769 |
| CNR1-Tango | 0.591 | −0.217 | 0.609 | −0.016 | 0.609 | −0.458 | −0.343 | −0.830 |
| CNR2-Tango | 0.082 | −0.178 | 0.211 | −0.058 | 0.160 | −0.029 | 0.290 | −0.781 |
| CRHR1-Tango | 0.226 | 0.310 | −0.029 | 0.155 | 0.372 | −0.160 | −0.623 | −1.622 |
| CRHR2-Tango | −0.445 | −0.515 | −0.311 | 0.378 | 0.077 | −0.532 | −0.197 | −0.348 |
| CX3C1-Tango | −0.194 | 0.057 | −0.471 | −0.451 | −0.639 | −0.445 | −0.105 | −0.926 |
| CXCR1-Tango | 0.517 | −0.308 | 0.619 | −0.110 | 0.337 | 0.654 | 0.377 | −1.007 |
| CXCR2-Tango | 1.466 | 0.331 | 1.330 | −0.117 | −0.463 | −0.118 | −0.114 | −1.540 |
| CXCR3-Tango | 0.215 | −0.384 | 0.340 | −0.084 | −0.063 | 0.362 | 0.079 | −1.257 |
| CXCR4-Tango | 0.108 | −0.123 | 0.343 | 0.393 | −0.167 | −0.205 | −0.224 | −0.895 |
| CXCR5-Tango | −0.050 | −0.661 | −0.014 | 0.260 | 0.035 | −0.226 | −0.010 | −1.013 |
| CXCR6-Tango | 0.028 | −0.564 | −0.067 | −0.050 | −0.028 | 0.624 | 0.970 | −1.442 |
| CXCR7-Tango | −0.043 | −0.334 | −0.027 | −0.555 | −0.430 | 0.173 | 0.232 | −1.875 |
| CYSLTR1-Tango | 0.790 | −0.314 | 0.905 | −0.494 | −0.555 | 0.081 | −0.128 | −1.761 |
| CYSLTR2-Tango | 0.054 | −0.715 | 0.184 | 0.000 | −0.133 | 0.500 | 0.340 | −1.013 |
| DRD1-Tango | −0.279 | −0.825 | −0.204 | −0.334 | −0.391 | 0.928 | 0.727 | −3.851 |
| DRD2-Tango | −0.597 | −1.570 | −0.574 | 0.628 | −0.319 | 0.731 | 0.617 | −1.992 |
| DRD3-Tango | 0.079 | −2.916 | 0.030 | 0.050 | 0.598 | 1.915 | 1.415 | −2.126 |
| DRD4-Tango | −0.322 | −3.000 | −0.052 | 0.554 | 0.114 | 1.065 | 0.874 | −1.032 |
| DRD5-Tango | −0.666 | −1.197 | −0.547 | −0.687 | −0.597 | −0.141 | 0.364 | −1.667 |
| EDNRA-Tango | −0.260 | −0.197 | −0.335 | −0.551 | −0.261 | −0.258 | 0.135 | −1.213 |
| EDNRB-Tango | 0.160 | −0.503 | 0.127 | −0.455 | −0.182 | −0.363 | −0.363 | −0.851 |
| ELTD1-Tango | −0.636 | −0.325 | −0.603 | −1.290 | −0.807 | −0.414 | −0.383 | −1.309 |
| F2R-Tango | −0.198 | −0.371 | −0.185 | −0.576 | −0.236 | −0.114 | −0.497 | −0.953 |
| F2RL1-Tango | −0.220 | −0.207 | −0.166 | −0.730 | 0.177 | −0.023 | −0.361 | −0.855 |
| F2RL2-Tango | −0.212 | −0.580 | −0.178 | −0.541 | 0.025 | −0.228 | −0.482 | −0.865 |
| F2RL3-Tango | −0.251 | −0.594 | −0.305 | −0.776 | −0.241 | −0.140 | −0.135 | −0.966 |
| FFA1-Tango | 0.009 | 0.110 | 0.055 | −0.349 | −0.069 | −0.607 | −0.629 | −1.057 |
| FFA2-Tango | −0.229 | −0.732 | −0.111 | −0.575 | −0.382 | −0.175 | −0.167 | −1.365 |
| FFA3-Tango | −0.051 | −0.682 | −0.136 | −0.078 | 0.032 | 0.020 | 0.175 | −0.763 |
| FPR1-Tango | −1.079 | −1.491 | −0.924 | −0.992 | −0.511 | 0.074 | 0.153 | −1.639 |
| FPR2-Tango | 1.469 | −0.237 | 1.608 | −0.251 | 0.535 | 0.420 | 0.286 | −0.904 |
| FPR3-Tango | 0.165 | −0.607 | 0.077 | −0.660 | −0.330 | −0.062 | −0.270 | −1.221 |
| FSHR-Tango | −0.289 | −1.236 | −0.408 | −0.479 | −0.343 | −0.382 | −0.305 | −0.682 |
| GABBR1-Tango | −0.067 | −1.129 | −0.132 | −0.310 | 0.129 | −0.260 | −0.260 | −0.577 |
| GAL1-Tango | −0.669 | −0.357 | 0.387 | 0.430 | 0.019 | −0.383 | −0.596 | −2.110 |
| GAL2-Tango | 0.302 | −0.772 | 0.211 | 0.213 | 0.000 | 1.657 | 0.791 | −0.823 |
| GAL3-Tango | −0.105 | −0.888 | 0.054 | −0.443 | 0.149 | −0.645 | −0.122 | −0.731 |
| GCGR-Tango | −0.280 | −0.415 | −0.237 | −0.435 | −0.258 | −0.471 | −0.111 | −0.713 |
| GHRHR-Tango | −0.302 | −1.177 | −0.302 | 0.053 | −0.430 | −0.575 | −0.728 | −1.271 |
| GHSR-Tango | −0.954 | −0.425 | −0.641 | −0.684 | −1.092 | −0.086 | −0.395 | −1.579 |
| GIPR-Tango | −0.703 | −0.835 | −0.538 | −0.324 | −0.787 | 0.000 | −0.385 | −0.998 |
| GLP1R-Tango | −0.894 | −0.759 | −0.674 | −0.333 | −0.387 | −0.099 | −0.475 | −1.094 |
| GLP2R-Tango | 0.395 | 0.014 | 0.475 | 0.339 | −0.152 | 0.633 | −0.100 | −1.090 |
| GNRHR-Tango | 0.143 | −0.628 | −0.040 | 0.304 | −0.173 | 0.420 | 0.094 | −1.115 |
| GPBA-Tango | 0.713 | −0.463 | 0.494 | 0.331 | 0.260 | 0.266 | 0.595 | −1.291 |
| GPER-Tango | −0.461 | −0.873 | −0.447 | −0.154 | −0.104 | 0.038 | 0.038 | −1.028 |
| GPR1-Tango | −0.189 | −0.699 | −0.064 | −0.295 | −0.556 | −0.289 | −0.387 | −1.174 |
| GPR101-Tango | 0.355 | −0.481 | 0.232 | −0.354 | −0.045 | 0.053 | 0.251 | −0.734 |
| GPR110-Tango | −0.331 | −0.858 | −0.364 | −0.717 | −0.543 | 0.085 | −0.024 | −0.758 |
| GPR111-Tango | −0.292 | −1.422 | −0.311 | −0.207 | 0.118 | 0.018 | −0.069 | −0.875 |
| GPR113-Tango | −0.487 | −0.446 | −0.371 | −0.669 | −1.174 | 0.149 | −0.243 | −0.952 |
| GPR114-Tango | −0.091 | −0.667 | −0.306 | −0.102 | −0.199 | −0.107 | 0.082 | −0.544 |
| GPR115-Tango | −0.279 | −0.335 | 0.020 | −0.601 | −0.415 | −0.440 | −0.059 | −0.626 |
| GPR116-Tango | −0.449 | −0.432 | −0.374 | −0.452 | −0.577 | −0.253 | −0.187 | −0.800 |
| GPR119-Tango | −0.006 | −0.788 | 0.010 | −0.129 | 0.034 | 0.141 | −0.073 | −0.862 |
| GPR12-Tango | −0.241 | −0.416 | 0.018 | −0.133 | −0.282 | −0.182 | −0.293 | −0.915 |
| GPR120-Tango | 0.162 | −0.728 | 0.246 | 0.045 | 0.063 | −0.136 | −0.355 | −0.708 |
| GPR123-Tango | −0.384 | −0.416 | −0.020 | 0.196 | −0.094 | −0.212 | 0.218 | −0.378 |
| GPR124-Tango | −0.237 | −0.417 | −0.193 | 0.079 | −0.021 | −0.233 | 0.289 | −1.465 |
| GPR125-Tango | 0.239 | −0.279 | −0.021 | −0.104 | 0.137 | 0.109 | 0.281 | −0.712 |
| GPR126-Tango | −0.016 | −1.030 | 0.086 | −0.411 | 0.151 | −0.184 | −0.325 | −0.765 |
| GPR132-Tango | −0.380 | −1.091 | −0.051 | −0.452 | −0.190 | −0.353 | −0.358 | 0.188 |
| GPR133-Tango | −0.242 | −0.576 | 0.024 | −0.323 | −0.140 | −0.251 | −0.474 | −0.365 |
| GPR135-Tango | −0.169 | −1.208 | −0.159 | −0.092 | 0.101 | 0.117 | −0.051 | −0.499 |
| GPR141-Tango | −0.056 | −0.276 | 0.079 | −0.251 | 0.030 | −0.385 | −0.369 | −0.688 |
| GPR142-Tango | −0.397 | −0.404 | −0.136 | −0.185 | −0.049 | 0.111 | −0.065 | −0.811 |
| GPR143-Tango | −0.225 | −0.515 | −0.333 | 0.027 | 0.147 | −0.072 | 0.414 | −1.074 |
| GPR144-Tango | −0.329 | −0.537 | −0.524 | 0.022 | −0.317 | −0.585 | −0.585 | −1.654 |
| GPR146-Tango | −0.118 | −0.525 | 0.222 | −0.037 | −0.043 | 0.066 | −0.472 | −1.363 |
| GPR148-Tango | 1.087 | −2.373 | 1.700 | 0.467 | 1.828 | −0.485 | 0.147 | −1.260 |
| GPR149-Tango | 0.419 | −0.374 | 0.519 | 0.723 | 0.858 | 0.310 | 0.208 | −1.836 |
| GPR15-Tango | −0.955 | −0.303 | −0.441 | 0.804 | 0.465 | −0.369 | 0.196 | −1.656 |
| GPR150-Tango | 0.558 | −0.350 | 0.680 | 0.118 | 0.558 | 0.813 | −0.122 | −1.377 |
| GPR151-Tango | 0.456 | 0.240 | 0.781 | 0.319 | 0.749 | −0.900 | −0.942 | −1.920 |
| GPR152-Tango | −0.089 | −0.023 | −0.060 | 0.034 | −0.007 | −0.471 | −0.239 | −0.642 |
| GPR153-Tango | 0.173 | −0.334 | 0.189 | 0.144 | 0.132 | −0.184 | −0.323 | −0.800 |
| GPR156-Tango | 0.095 | −0.455 | 0.128 | 0.273 | −0.035 | 0.078 | 0.007 | −0.723 |
| GPR157-Tango | 0.114 | −0.523 | 0.370 | 0.227 | 0.370 | −0.113 | −0.375 | −0.765 |
| GPR158-Tango | 0.014 | −0.358 | 0.136 | 0.103 | 0.506 | −0.006 | 0.077 | −0.558 |
| GPR160-Tango | 0.243 | −0.038 | 0.313 | 0.311 | 0.574 | −0.005 | −0.284 | −0.430 |
| GPR161-Tango | 0.347 | −0.022 | 0.471 | 0.442 | 0.454 | −0.392 | −0.120 | −0.484 |
| GPR162-Tango | −0.139 | −1.232 | 0.034 | −0.176 | −0.009 | −0.354 | −0.136 | −0.646 |
| GPR17-Tango | 0.189 | 0.326 | 0.265 | 0.412 | −0.078 | 0.095 | 0.238 | −1.204 |
| GPR171-Tango | 0.050 | −0.297 | 0.207 | 0.583 | 0.078 | 0.013 | −0.179 | −0.806 |
| GPR173-Tango | 0.054 | −0.231 | 0.467 | 0.082 | 0.196 | −0.265 | −0.155 | −0.783 |
| GPR174-Tango | 0.029 | 0.068 | 0.543 | 0.145 | 0.086 | −0.097 | 0.000 | −0.753 |
| GPR18-Tango | 0.403 | −0.008 | 0.609 | 0.668 | 0.510 | −0.239 | −0.224 | −0.956 |
| GPR182-Tango | −0.019 | −0.204 | 0.601 | 0.307 | −0.052 | −0.654 | −0.519 | −0.861 |
| GPR183-Tango | −0.170 | −0.711 | 0.330 | 0.270 | 0.140 | −0.460 | −0.518 | −0.678 |
| GPR19-Tango | −0.477 | −0.372 | 0.175 | 0.189 | 0.002 | −0.858 | −0.941 | −0.608 |
| GPR20-Tango | 0.675 | −0.585 | 0.128 | 0.136 | 0.652 | 0.170 | −0.368 | −0.785 |
| GPR21-Tango | 0.271 | −1.250 | 0.076 | 0.488 | 0.301 | 0.000 | −0.345 | −0.931 |
| GPR22-Tango | 0.109 | −0.376 | 0.204 | 0.209 | 0.479 | 0.094 | 0.173 | −0.508 |
| GPR25-Tango | −0.242 | −1.126 | 0.169 | 0.147 | 0.320 | 0.000 | 0.000 | −0.797 |
| GPR26-Tango | −0.013 | −0.064 | 0.182 | 0.169 | 0.659 | −0.214 | −0.255 | −1.004 |
| GPR27-Tango | −0.036 | −0.745 | 0.236 | 0.402 | 0.150 | 0.468 | 0.303 | −0.628 |
| GPR3-Tango | 0.229 | 0.113 | 0.330 | −0.018 | 0.307 | −0.195 | 0.025 | −0.829 |
| GPR31-Tango | −0.148 | −0.492 | 0.181 | −0.006 | 0.193 | −0.289 | −0.080 | −1.281 |
| GPR32-Tango | −0.402 | −0.470 | −0.276 | −0.367 | −0.775 | −0.040 | −0.040 | −1.875 |
| GPR34-Tango | −0.246 | −1.089 | −0.068 | −0.229 | −0.455 | 0.039 | −0.379 | −1.474 |
| GPR35-Tango | −0.229 | −0.608 | −0.085 | −0.158 | −0.647 | −0.245 | 0.060 | −1.435 |
| GPR37-Tango | −0.307 | −0.732 | 0.129 | −0.131 | −0.512 | −0.110 | −0.072 | −1.072 |
| GPR37L1-Tango | −0.257 | −0.241 | −0.329 | 0.142 | 0.117 | −0.249 | −0.114 | −0.873 |
| GPR39-Tango | −0.349 | −0.500 | −0.431 | −0.481 | −0.383 | −0.756 | −0.332 | −0.762 |
| GPR4-Tango | −0.221 | −0.602 | −0.069 | −0.015 | −0.446 | 0.039 | 0.069 | −0.599 |
| GPR44-Tango | −0.269 | −0.792 | −0.391 | −0.182 | −0.297 | 0.000 | −0.126 | −0.667 |
| GPR45-Tango | −0.649 | −0.451 | −0.549 | −0.463 | −0.757 | −0.260 | −0.824 | −0.941 |
| GPR50-Tango | 0.265 | −0.540 | 0.418 | 0.858 | 0.358 | 0.249 | 0.092 | −1.234 |
| GPR52-Tango | 0.211 | −0.971 | 0.165 | 0.406 | −0.030 | 0.178 | −0.135 | −0.840 |
| GPR55-Tango | −0.293 | −1.228 | 0.002 | 0.388 | −0.192 | −0.190 | −0.328 | −1.023 |
| GPR56-Tango | −0.116 | −0.486 | 0.006 | 0.716 | 0.180 | −0.384 | 0.222 | −0.955 |
| GPR6-Tango | 0.186 | −0.389 | 0.256 | 0.272 | −0.160 | −0.147 | −0.876 | −1.210 |
| GPR61-Tango | −0.224 | −0.276 | 0.039 | 0.635 | −0.162 | −0.402 | −0.646 | −0.665 |
| GPR62-Tango | 0.027 | −0.477 | 0.262 | 0.457 | −0.062 | −0.527 | −0.740 | −0.718 |
| GPR63-Tango | −0.063 | −0.225 | −0.027 | −0.301 | −0.236 | −0.414 | 0.000 | −0.268 |
| GPR64-Tango | −0.490 | −0.569 | −0.443 | −0.049 | −0.554 | 0.039 | −0.040 | −0.543 |
| GPR65-Tango | −0.182 | −0.178 | −0.043 | −0.174 | −0.155 | −0.313 | −0.441 | −0.513 |
| GPR68-Tango | −0.597 | −0.860 | −0.571 | −0.450 | −0.366 | −0.103 | −0.546 | −0.622 |
| GPR75-Tango | −0.147 | −0.423 | −0.062 | 0.360 | 0.185 | −0.168 | −0.309 | −0.313 |
| GPR77-Tango | −0.999 | −1.201 | −0.605 | −0.058 | −0.818 | −0.872 | −0.941 | −1.169 |
| GPR78-Tango | 0.104 | −0.459 | 0.238 | 0.596 | 0.063 | −0.755 | −0.578 | −0.623 |
| GPR82-Tango | −0.611 | 0.077 | −0.175 | −0.033 | −0.569 | −0.306 | 0.011 | −1.337 |
| GPR83-Tango | −0.352 | −0.444 | −0.239 | −0.017 | −0.279 | −0.176 | −0.481 | −0.740 |
| GPR84-Tango | −0.438 | −0.220 | −0.235 | 0.006 | −0.420 | −0.816 | −0.813 | −0.840 |
| GPR85-Tango | −0.231 | −0.519 | −0.199 | 0.368 | −0.042 | −0.443 | −0.261 | −0.814 |
| GPR87-Tango | −0.082 | 0.012 | 0.047 | 0.333 | −0.268 | −0.157 | −0.524 | −0.942 |
| GPR88-Tango | −0.044 | −1.054 | 0.171 | 0.090 | 0.000 | 0.223 | 0.434 | −0.796 |
| GPR97-Tango | −0.407 | −0.779 | −0.216 | 0.161 | −0.540 | −0.110 | −0.767 | −0.510 |
| GPRC5A-Tango | −0.665 | −0.515 | −0.287 | 0.093 | −0.582 | −0.711 | −0.920 | −0.738 |
| GPRC5B-Tango | −0.195 | −0.397 | 0.017 | 0.233 | −0.226 | −0.780 | −0.594 | −0.565 |
| GPRC5C-Tango | −0.275 | −0.214 | −0.262 | 0.215 | −0.222 | −0.409 | −0.387 | −0.077 |
| GPRC5D-Tango | −0.426 | −0.641 | −0.478 | −0.209 | −0.451 | 0.000 | −0.494 | −0.493 |
| GPRC6A-Tango | −0.520 | −0.671 | −0.318 | −0.186 | −0.458 | −0.559 | −0.737 | −0.653 |
| GRM1-Tango | −0.624 | −0.498 | −0.632 | −0.060 | −0.309 | −0.324 | −0.441 | −0.750 |
| GRM2-Tango | −0.414 | −1.138 | −0.241 | 0.000 | −0.466 | −0.462 | −0.510 | −0.567 |
| GRM4-Tango | −0.637 | −1.282 | −0.434 | −0.050 | −0.478 | −0.542 | −0.288 | −0.328 |
| GRM5-Tango | −0.139 | −1.161 | −0.093 | −0.428 | −0.024 | −0.746 | −0.589 | 0.143 |
| GRM6-Tango | −0.421 | −0.620 | −0.555 | 0.514 | −0.501 | −0.379 | −0.404 | −1.185 |
| GRM7-Tango | −0.769 | −0.486 | −0.655 | −0.725 | −1.030 | −0.309 | −0.099 | −0.705 |
| GRM8-Tango | −0.663 | −0.857 | −0.505 | −0.589 | −0.886 | −0.219 | −0.147 | −0.864 |
| GRPR-Tango | −0.295 | −0.515 | 0.067 | −0.227 | −0.234 | −0.094 | −0.150 | −0.518 |
| HCA1-Tango | −0.113 | 0.290 | 0.218 | 0.373 | 0.143 | −0.399 | −0.399 | −0.825 |
| HCA2-Tango | −0.061 | 0.175 | 0.761 | 0.119 | 0.414 | 0.131 | −0.087 | −0.670 |
| HCA3-Tango | −0.108 | 0.178 | −0.100 | −0.082 | 0.056 | 0.235 | 0.033 | −0.910 |
| HCTR1-Tango | 0.000 | 0.745 | 0.000 | −0.181 | 0.258 | 0.369 | −1.778 | −1.122 |
| HCTR2-Tango | 0.435 | 0.000 | −0.395 | 0.451 | 0.435 | 0.122 | 0.234 | −0.754 |
| HRH1-Tango | 0.264 | −0.194 | 0.486 | −0.537 | −0.113 | −0.203 | −0.060 | −1.351 |
| HRH2-Tango | 0.154 | −0.607 | 0.180 | 0.176 | 0.006 | 0.417 | 0.389 | −0.707 |
| HRH3-Tango | −0.022 | −0.744 | −0.140 | 0.658 | 0.201 | 0.097 | 0.343 | −0.793 |
| HRH4-Tango | 0.283 | −0.675 | 0.300 | 0.164 | 0.361 | 0.659 | 0.431 | −1.142 |
| HTR1A-Tango | 0.091 | −0.340 | 0.269 | 0.044 | 0.174 | 0.174 | 0.000 | −0.760 |
| HTR1B-Tango | 0.088 | −0.426 | 0.093 | −0.487 | 0.069 | 0.066 | −0.102 | −1.133 |
| HTR1D-Tango | −0.054 | −0.272 | 0.088 | −0.592 | −0.060 | −0.405 | −0.254 | −1.155 |
| HTR1E-Tango | 0.093 | 0.033 | 0.001 | −0.006 | −0.257 | −0.411 | −0.251 | −0.895 |
| HTR1F-Tango | −0.531 | −0.696 | −0.349 | −0.802 | −0.806 | −0.214 | −0.214 | −1.566 |
| HTR2A-Tango | −0.345 | −1.112 | −0.472 | −0.453 | −0.501 | −0.065 | −0.114 | −0.780 |
| HTR2B-Tango | −0.176 | −1.200 | −0.097 | −0.439 | −0.213 | −0.028 | 0.115 | −0.743 |
| HTR2C-Tango | −0.233 | −1.471 | −0.262 | −0.062 | −0.318 | −0.570 | −0.432 | −1.034 |
| HTR4-Tango | −0.240 | −0.761 | −0.159 | −0.311 | −0.622 | 0.080 | 0.070 | −0.725 |
| HTR5-Tango | −0.504 | −0.169 | −0.085 | −0.050 | −0.968 | 0.205 | 0.227 | −0.234 |
| HTR6-Tango | 0.313 | −0.707 | 0.365 | 0.000 | −0.307 | −0.413 | −0.476 | 0.061 |
| HTR7-Tango | 0.339 | −2.319 | 0.395 | 0.630 | 0.501 | −0.189 | −0.416 | 0.539 |
| KISSPEPTIN-R- | −0.020 | −0.596 | −0.041 | −0.234 | −0.275 | −0.065 | −0.333 | −0.995 |
| Tango | | | | | | | | |
| LHCGR-Tango | −0.494 | −0.183 | −0.574 | −0.357 | −0.671 | −0.149 | −0.379 | −0.810 |
| LPAR1-Tango | 0.027 | −0.272 | 0.173 | −0.419 | −0.560 | −0.273 | −0.210 | −0.669 |
| LPAR2-Tango | −0.271 | −0.637 | −0.144 | −0.260 | −0.235 | −0.817 | −1.056 | −0.815 |
| LPAR4-Tango | −0.188 | −0.694 | −0.190 | −0.111 | −0.864 | −0.747 | −0.980 | −1.101 |
| LPAR5-Tango | −0.180 | −0.464 | −0.046 | 0.006 | −0.319 | −0.155 | −0.493 | −0.946 |
| LPAR6-Tango | 0.243 | 0.370 | 0.333 | 0.161 | 0.174 | −0.025 | −0.098 | −0.387 |
| LTB4R-Tango | 0.000 | −0.020 | 0.211 | 0.414 | −0.082 | −0.133 | −0.313 | −0.887 |
| LTB4R2B-Tango | 0.073 | 0.011 | 0.461 | −0.478 | 0.232 | −0.190 | −0.540 | −0.912 |
| MAS1-Tango | −0.164 | −0.496 | 0.220 | −0.280 | −0.244 | −0.470 | −0.717 | −1.686 |
| MAS1L-Tango | −0.071 | −0.678 | 0.151 | −0.614 | −0.313 | −0.717 | −0.826 | −0.946 |
| MC1R-Tango | −0.228 | −0.723 | 0.260 | −0.122 | −0.195 | −0.276 | −0.313 | −0.767 |
| MC2R-Tango | −0.382 | −1.036 | −0.019 | 0.059 | −0.382 | 0.458 | 0.393 | −0.736 |
| MC3R-Tango | 0.013 | −0.560 | 0.434 | 0.288 | 0.389 | −0.540 | −0.285 | −0.608 |
| MC4R-Tango | −0.307 | 0.225 | 0.334 | 0.587 | −0.648 | −0.397 | −0.295 | −1.194 |
| MC5R-Tango | −0.079 | 0.096 | 0.774 | 0.261 | −0.346 | −0.672 | −0.180 | −1.253 |
| MCHR1-Tango | −0.476 | −0.172 | −0.551 | 0.032 | 0.068 | −0.578 | −0.174 | −1.137 |
| MCHR2-Tango | −0.214 | −0.962 | −0.177 | 0.190 | −0.167 | 0.026 | −0.629 | −0.896 |
| MLNR-Tango | 0.395 | −0.691 | 0.010 | 0.295 | 0.135 | −0.170 | 0.132 | −0.817 |
| MRGPRD-Tango | −0.069 | −0.196 | −0.024 | 0.185 | −0.317 | −0.396 | −0.321 | −1.075 |
| MRGPRE-Tango | 0.043 | −0.326 | −0.165 | 0.488 | −0.224 | −0.190 | −0.321 | −0.510 |
| MRGPRF-Tango | −0.377 | 0.216 | −0.203 | 0.496 | −0.173 | −0.183 | −0.172 | −0.620 |
| MRGPRG-Tango | −0.030 | 0.076 | −0.240 | −0.219 | 0.051 | −0.398 | −0.294 | −0.127 |
| MRGPRX1-Tango | −0.272 | 0.453 | −0.302 | −0.290 | −0.221 | −0.137 | −0.151 | −0.473 |
| MRGPRX2-Tango | −0.183 | −0.820 | −0.127 | 0.048 | −0.986 | 0.012 | −0.404 | −1.182 |
| MRGPRX3-Tango | −0.457 | −1.941 | −0.482 | 0.069 | −0.661 | −0.079 | −0.298 | −0.998 |
| MRGPRX4-Tango | −0.983 | −1.258 | −0.779 | −0.836 | −1.240 | −0.312 | −0.209 | −1.770 |
| MTNR1A-Tango | −0.743 | −0.621 | −0.483 | −0.265 | −1.072 | −0.737 | −0.120 | −1.790 |
| MTNR1B-Tango | −0.465 | −0.334 | −0.254 | −0.090 | −1.011 | −0.188 | −0.490 | −1.645 |
| NMBR-Tango | −0.572 | −0.320 | −0.160 | 0.542 | −0.594 | −0.480 | −0.664 | −0.667 |
| NMUR1-Tango | −0.589 | 0.148 | −0.206 | −0.090 | −0.820 | −0.523 | −0.650 | −1.423 |
| NMUR2-Tango | −0.895 | −0.290 | −0.534 | 0.085 | −0.681 | −0.420 | −0.122 | −0.915 |
| NPBW1-Tango | 0.142 | −1.887 | 0.080 | 0.437 | 0.059 | 0.133 | 0.403 | −1.022 |
| NPBW2-Tango | −0.107 | −1.538 | −0.256 | −0.594 | −0.521 | −0.245 | −0.436 | −1.406 |
| NPFF1-Tango | 0.056 | −1.303 | 0.138 | 0.257 | 0.071 | −0.506 | −0.336 | −0.925 |
| NPFF2-Tango | 0.118 | −0.825 | 0.293 | 0.571 | 0.404 | −0.229 | −0.353 | −0.544 |
| NPS-Tango | −0.311 | −0.817 | 0.257 | 0.155 | 0.300 | −0.427 | −0.766 | −0.951 |
| NPY1R-Tango | −0.015 | −0.788 | 0.067 | 0.039 | 0.219 | −0.632 | −1.016 | −0.831 |
| NPY2R-Tango | −0.214 | −1.710 | −0.189 | 0.357 | −0.492 | −0.543 | −0.589 | −0.386 |
| NPY4R-Tango | −0.240 | −0.900 | −0.429 | 0.295 | −0.154 | 0.379 | −0.411 | −0.527 |
| NPY5R-Tango | −0.404 | −1.029 | −0.088 | 0.459 | 0.267 | 0.120 | 0.000 | −0.761 |
| NTSR1-Tango | −0.910 | −0.773 | −0.197 | −0.075 | −0.292 | −0.184 | −0.606 | −1.006 |
| NTSR2-Tango | −0.368 | −0.326 | −0.023 | −0.464 | 0.239 | −0.276 | −0.222 | −1.288 |
| OPN3-Tango | −0.290 | −1.384 | −0.246 | 0.492 | −0.237 | −0.518 | −0.225 | −1.073 |
| OPN5-Tango | −0.681 | −0.698 | −0.279 | −0.339 | −0.589 | −0.369 | −0.478 | −1.546 |
| OPRD1-Tango | −0.617 | −0.740 | −0.445 | −0.248 | −0.463 | 0.142 | −0.219 | −1.299 |
| OPRK1-Tango | 0.539 | −0.369 | 0.408 | −0.094 | 0.541 | −0.214 | −0.473 | −0.506 |
| OPRL1-Tango | 0.000 | −0.793 | 0.031 | −0.108 | −0.024 | 0.376 | −0.624 | −0.455 |
| OPRM1-Tango | −0.383 | −1.009 | −0.369 | 0.117 | −0.098 | −0.903 | −0.225 | −0.883 |
| OXER1-Tango | −0.244 | −0.093 | −0.354 | −0.182 | 0.000 | −0.348 | −0.402 | −0.727 |
| OXGR1-Tango | −1.097 | 0.156 | −0.718 | −0.384 | −1.154 | −0.245 | −0.620 | −1.871 |
| OXTR-Tango | −0.192 | −1.257 | −0.124 | 0.109 | −0.074 | −0.191 | −0.429 | −0.946 |
| P2RY1-Tango | 0.518 | −0.549 | 0.222 | −0.569 | −0.211 | −0.266 | −0.352 | −1.305 |
| P2RY10-Tango | −0.227 | −0.965 | −0.138 | −0.416 | −0.210 | −0.333 | −0.314 | −0.697 |
| P2RY11-Tango | −0.126 | −0.878 | −0.102 | −0.623 | −0.206 | −0.807 | −0.178 | −0.881 |
| P2RY12-Tango | −0.618 | −0.740 | −0.278 | 0.099 | −1.306 | 0.346 | 0.065 | −1.075 |
| P2RY13-Tango | 0.144 | −0.509 | 0.242 | −0.068 | −0.471 | 0.159 | −0.096 | −1.524 |
| P2RY14-Tango | −0.106 | −1.020 | −0.145 | −0.120 | −0.476 | −0.115 | 0.122 | −1.354 |
| P2RY2-Tango | −0.387 | −0.826 | 0.038 | −0.310 | −0.547 | −0.221 | −0.424 | −1.495 |
| P2RY4-Tango | −0.414 | −0.734 | −0.319 | −0.571 | −0.889 | −0.129 | −0.320 | −1.464 |
| P2RY6-Tango | −0.224 | −0.406 | −0.089 | −0.114 | −0.243 | 0.103 | 0.035 | −1.687 |
| P2RY8-Tango | −0.229 | −0.569 | −0.320 | −0.418 | −0.626 | 0.145 | 0.016 | −0.779 |
| PK1-Tango | 0.078 | −0.638 | −0.024 | 0.079 | −0.057 | −0.493 | −0.128 | −0.831 |
| PK2-Tango | 0.045 | 0.087 | −0.230 | 0.362 | −0.073 | −0.258 | −0.258 | −0.778 |
| PRRP-Tango | −0.181 | −0.493 | −0.013 | 0.320 | −0.041 | 0.436 | 0.160 | −1.008 |
| PTAFR-Tango | −0.181 | −0.655 | −0.071 | 0.233 | −0.348 | −0.125 | 0.070 | −0.872 |
| PTGDR-Tango | −0.114 | −1.135 | −0.210 | −0.181 | −0.382 | −0.166 | −0.339 | −0.890 |
| PTGER1-Tango | 0.084 | −0.794 | −0.093 | 0.074 | 0.103 | 0.085 | −0.542 | −0.720 |
| PTGER2-Tango | −0.118 | −0.554 | 0.034 | 0.331 | 0.080 | 0.272 | −0.054 | −0.787 |
| PTGER3-Tango | −0.135 | 0.034 | −0.100 | −0.178 | −0.574 | −0.090 | −0.173 | −0.768 |
| PTGER4-Tango | 0.473 | 0.409 | 0.598 | 0.701 | 0.612 | −0.114 | −0.260 | −0.325 |
| PTGFR-Tango | −0.036 | −0.150 | 0.026 | −0.427 | −0.623 | 0.260 | 0.060 | −0.976 |
| PTGIR-Tango | −0.084 | 0.616 | 0.381 | 0.155 | −0.194 | 0.813 | 0.376 | −0.586 |
| PTH1R-Tango | −0.356 | −0.611 | −0.290 | 0.063 | −0.236 | 0.317 | −0.267 | −0.755 |
| PTH2R-Tango | −0.260 | 0.068 | −0.522 | 0.165 | −0.272 | −0.281 | −0.274 | −0.741 |
| QRFP-Tango | −0.291 | −0.367 | −0.096 | 0.095 | −0.102 | −0.142 | 0.214 | −0.851 |
| RXFP1-Tango | −0.117 | −0.146 | −0.280 | −1.021 | −0.244 | −0.602 | −0.326 | −0.542 |
| RXFP2-Tango | −0.655 | −0.229 | −0.756 | −0.678 | −0.700 | −0.067 | 0.019 | −0.778 |
| RXFP3-Tango | −0.621 | −0.227 | −0.585 | 0.035 | −0.595 | −0.910 | −0.768 | −0.576 |
| RXFP4-Tango | −0.737 | −0.270 | −0.280 | −0.145 | −0.168 | −0.317 | −0.565 | −0.567 |
| S1PR1-Tango | −0.058 | 1.015 | −0.044 | 0.526 | −0.246 | −0.793 | −0.458 | −0.333 |
| S1PR2-Tango | −0.204 | 0.515 | −0.106 | −0.294 | −0.598 | −0.142 | −0.519 | −0.949 |
| S1PR3-Tango | 0.044 | 0.684 | −0.171 | 0.158 | −0.316 | −0.545 | −0.027 | −0.074 |
| S1PR4-Tango | −0.732 | 0.235 | −0.405 | 0.127 | −1.071 | −0.484 | −0.211 | −1.523 |
| S1PR5-Tango | −0.073 | 0.229 | 0.250 | 1.019 | −0.087 | −1.000 | 0.000 | −0.748 |
| SCTR-Tango | 0.421 | 0.346 | 0.062 | 0.243 | 0.176 | 0.115 | 0.115 | −0.872 |
| SSTR1-Tango | −0.539 | 0.613 | −0.476 | 0.087 | −0.030 | −1.404 | −0.170 | −0.778 |
| SSTR2-Tango | −0.344 | 0.000 | 0.069 | 0.312 | 0.436 | −1.766 | −0.333 | −0.503 |
| SSTR3-Tango | −0.025 | −0.181 | 0.065 | 0.096 | 0.065 | 0.697 | 0.463 | −0.615 |
| SSTR4-Tango | −0.378 | −0.193 | −0.554 | 0.510 | −0.210 | −0.322 | 0.243 | −0.984 |
| SSTR5-Tango | 0.769 | −0.678 | 0.804 | 1.110 | −0.023 | 0.269 | −0.111 | −0.153 |
| SUCNR1-Tango | 0.363 | −0.044 | 0.222 | 0.825 | −0.070 | 0.097 | 0.160 | 3.083 |
| TA1-Tango | −0.142 | −0.533 | −0.113 | 0.787 | 0.016 | 0.288 | 0.384 | −0.582 |
| TAAR2-Tango | −0.180 | −0.413 | 0.016 | 0.036 | −0.061 | 0.070 | −0.304 | −0.208 |
| TAAR5-Tango | −0.166 | −0.892 | −0.323 | −0.223 | −0.228 | −0.513 | −0.483 | −1.021 |
| TAAR6-Tango | −0.488 | −0.402 | −0.598 | −0.261 | −0.629 | −0.540 | −0.652 | −0.670 |
| TAAR8-Tango | −0.333 | −0.323 | −0.467 | −0.184 | −0.524 | −0.130 | −0.238 | −0.364 |
| TAAR9-Tango | −0.457 | −0.537 | −0.362 | −0.301 | −0.314 | −0.053 | −0.104 | −0.360 |
| TACR1-Tango | −0.272 | −0.338 | −0.127 | −0.015 | −0.482 | −0.026 | 0.120 | −0.686 |
| TACR2-Tango | 0.045 | −0.081 | 0.039 | 0.111 | −0.201 | −0.491 | −0.421 | −0.523 |
| TACR3-Tango | −0.175 | −0.311 | −0.139 | 0.477 | −0.632 | −0.374 | −0.382 | −0.889 |
| TBXA2R-Tango | −0.262 | −0.968 | −0.402 | 0.233 | −0.298 | 0.026 | −0.728 | −0.746 |
| TSHR-Tango | −0.179 | −0.598 | −0.404 | −0.111 | −0.154 | −0.772 | −1.092 | −0.809 |
| UTS2R-Tango | −0.024 | −0.216 | 0.043 | 0.201 | −0.218 | −0.969 | −1.212 | −0.870 |
| VIPR1-Tango | −0.505 | −0.124 | −0.478 | 0.582 | −0.419 | 0.039 | −0.353 | −0.999 |
| VIPR2-Tango | −0.360 | −0.455 | 0.874 | 1.247 | 1.049 | 1.505 | 1.675 | −0.806 |

| PRESTO-Tango | Bacteroids | Bacteroids | Bacteroids | Bacteroids | Bacteroids | Bacteroids | Bacteroids | Bacteroids |
|---|---|---|---|---|---|---|---|---|
| with GMM | fragilis-2# | fragilis-3# | fragilis-4# | fragilis-5# | fragilis-6# | fragilis-7# | fragilis-8# | fragilis-9# |
| ADCYAP1R1- | −0.613 | −0.388 | −0.138 | −0.993 | −0.768 | −0.554 | −0.156 | 0.504 |
| Tango | | | | | | | | |
| ADORA1-Tango | −1.597 | −0.662 | −2.070 | −1.714 | −0.999 | −0.852 | −1.339 | −0.455 |
| ADORA2A-Tango | −1.013 | −0.062 | −0.967 | −0.290 | −0.172 | 0.256 | −0.733 | 0.487 |
| ADORA2B-Tango | −0.541 | 0.007 | 0.157 | −0.605 | −0.202 | 0.084 | −0.409 | 0.156 |
| ADORA3-Tango | −0.975 | 0.472 | 0.563 | −0.607 | −0.250 | −0.198 | −0.284 | 0.435 |
| ADRA1A-Tango | −0.619 | 0.040 | −0.078 | −0.797 | −0.071 | −0.060 | −0.325 | 0.477 |
| ADRA1B-Tango | −1.350 | −0.036 | −0.108 | −1.019 | −0.269 | −0.278 | −0.410 | −0.108 |
| ADRA1D-Tango | −0.913 | −0.913 | −0.712 | −0.708 | −0.431 | −0.225 | −0.754 | −0.224 |
| ADRA2A-Tango | −0.346 | 1.298 | −0.036 | −1.064 | −1.170 | −0.488 | −0.677 | 0.106 |
| ADRA2B-Tango | −1.585 | −0.131 | −1.251 | −1.034 | −1.287 | −1.677 | −0.958 | −0.235 |
| ADRA2C-Tango | 0.313 | 0.255 | 0.196 | −0.115 | −0.678 | −0.415 | −0.345 | −0.463 |
| ADRB1-Tango | −0.266 | 0.180 | −0.106 | −0.954 | −0.564 | −0.435 | −0.426 | −0.300 |
| ADRB2-Tango | −0.993 | −0.241 | −0.788 | −1.068 | −0.754 | −0.595 | −0.363 | 0.350 |
| ADRB3-Tango | −0.141 | −0.174 | −0.174 | −0.361 | −0.436 | 0.219 | 0.000 | −1.094 |
| AGTR1-Tango | −0.517 | −0.292 | −0.508 | −0.505 | −0.056 | 0.041 | 0.184 | 0.742 |
| AGTR2-Tango | −0.455 | 0.149 | 0.099 | −1.348 | −0.359 | −0.705 | −0.341 | −0.063 |
| APJ-Tango | −1.541 | −0.211 | −0.575 | −1.221 | −0.451 | −0.655 | −1.040 | −0.277 |
| AVPR1A-Tango | −1.098 | −0.034 | −1.293 | −0.863 | −0.273 | −0.021 | −0.918 | −0.436 |
| AVPR1B-Tango | −0.973 | −0.053 | −0.511 | −1.277 | −0.290 | −0.557 | −0.339 | −0.147 |
| AVPR2-Tango | −1.156 | −0.184 | −0.755 | −1.659 | −0.685 | −0.691 | −1.075 | −0.336 |
| BB3-Tango | −1.552 | −0.789 | −1.112 | −1.397 | −0.849 | −0.734 | −0.923 | −1.273 |
| BDKBR1-Tango | −0.364 | −0.146 | −0.520 | −0.947 | −0.412 | 0.024 | −0.746 | −0.333 |
| BDKBR2-Tango | −0.717 | 0.266 | −0.126 | −1.209 | −0.036 | −0.107 | 0.034 | 0.052 |
| C3AR1-Tango | −0.755 | −0.337 | −0.455 | −1.347 | −0.678 | −0.386 | −0.420 | −0.535 |
| C5A-Tango | −0.757 | −0.002 | −0.153 | −0.612 | 0.254 | −0.094 | −0.122 | −0.194 |
| CALCRb-Tango | −0.290 | 0.016 | −0.380 | −0.448 | −0.034 | −0.103 | −0.397 | −0.505 |
| CALCRL-Tango | 0.009 | −0.009 | −0.261 | −0.265 | −0.498 | 0.236 | −0.354 | −0.425 |
| CASR-Tango | −0.932 | −0.286 | −0.320 | −0.928 | −0.548 | −0.086 | −0.244 | −1.303 |
| CCKAR-Tango | −0.819 | −0.012 | 0.029 | −0.654 | −0.109 | −0.051 | −0.003 | −0.422 |
| CCKBR-Tango | −0.777 | −0.360 | −0.427 | −0.702 | −0.366 | −0.231 | −0.732 | −1.005 |
| CCR10-Tango | −0.813 | −0.049 | −0.511 | −0.703 | −0.724 | −0.392 | −0.744 | −0.752 |
| CCR2-Tango | −0.849 | −0.342 | −0.449 | −0.386 | −0.135 | −0.289 | −0.606 | −0.748 |
| CCR3-Tango | −1.572 | −0.545 | −1.136 | −2.224 | −0.064 | −0.515 | −0.689 | 0.801 |
| CCR4-Tango | −1.263 | 0.045 | −0.242 | −1.173 | −0.408 | 0.314 | 0.032 | −0.318 |
| CCR5-Tango | −0.841 | 0.016 | −0.281 | −0.930 | −0.182 | 0.000 | −0.139 | 0.227 |
| CCR6-Tango | −0.921 | 0.210 | 0.143 | −1.218 | 0.193 | −0.149 | 0.000 | 0.108 |
| CCR7-Tango | −1.052 | −0.167 | −0.447 | −0.503 | −0.847 | 0.360 | 0.120 | 0.108 |
| CCR8-Tango | −1.481 | −0.030 | −0.687 | −0.553 | −0.776 | −0.435 | −0.477 | 0.106 |
| CCRL2-Tango | −0.426 | 0.362 | 0.275 | −0.126 | −0.441 | 0.015 | 0.000 | 0.310 |
| CD97-Tango | −0.680 | −0.435 | 0.089 | −1.050 | −0.985 | −0.688 | −1.284 | −0.543 |
| CHRM1-Tango | −0.588 | 0.276 | −0.153 | 0.040 | 0.195 | −0.246 | −0.754 | −0.209 |
| CHRM2-Tango | −0.695 | −0.209 | −0.232 | −0.846 | −0.765 | −0.525 | −0.750 | −1.267 |
| CHRM3-Tango | −0.222 | 0.039 | −0.033 | −0.503 | −0.787 | −0.111 | −0.742 | −0.226 |
| CHRM4-Tango | 0.036 | −0.027 | −0.183 | −0.362 | −0.324 | 0.126 | 0.214 | −0.060 |
| CHRM5-Tango | −0.436 | −0.148 | −0.290 | −0.982 | −0.651 | −0.264 | 0.220 | −0.539 |
| CMKLR1-Tango | −0.431 | −0.032 | −0.326 | −0.732 | −0.230 | −0.062 | −0.331 | −0.390 |
| CNR1-Tango | −0.867 | 0.046 | −0.522 | −0.855 | −0.605 | −0.209 | −0.598 | −0.189 |
| CNR2-Tango | −0.843 | −0.053 | −0.727 | −0.876 | −0.616 | −0.224 | −0.748 | 0.335 |
| CRHR1-Tango | −0.434 | 0.029 | −0.229 | −0.957 | −0.186 | −0.415 | −1.211 | 0.675 |
| CRHR2-Tango | −0.348 | 0.262 | 0.039 | −0.745 | −0.011 | −0.588 | −0.475 | 0.905 |
| CX3C1-Tango | −0.750 | −0.370 | −0.775 | −0.595 | −0.105 | −0.302 | −0.230 | −0.142 |
| CXCR1-Tango | −0.950 | −0.551 | −0.602 | −0.235 | −0.809 | 0.103 | 0.125 | 0.620 |
| CXCR2-Tango | 0.049 | −0.043 | −0.314 | −0.613 | −0.768 | −0.804 | −0.282 | 0.540 |
| CXCR3-Tango | −0.983 | −0.090 | −0.366 | −0.578 | −0.442 | −0.309 | −0.366 | 0.485 |
| CXCR4-Tango | −0.200 | −0.027 | −0.086 | −1.078 | −0.553 | −0.231 | 0.026 | 0.541 |
| CXCR5-Tango | 0.231 | 0.221 | 0.147 | −0.673 | −0.322 | 0.105 | −0.187 | 0.094 |
| CXCR6-Tango | −0.705 | −0.459 | −0.372 | −0.871 | −0.174 | −1.007 | −0.105 | 0.719 |
| CXCR7-Tango | −1.436 | −0.724 | −0.676 | −1.624 | −1.236 | −1.089 | −1.271 | −0.121 |
| CYSLTR1-Tango | −1.168 | −0.713 | −0.698 | −0.820 | −0.337 | −0.473 | −0.074 | 0.253 |
| CYSLTR2-Tango | −0.490 | 0.532 | −0.188 | −0.075 | −0.523 | −0.097 | −0.024 | 0.055 |
| DRD1-Tango | −2.149 | −0.785 | −1.190 | −1.293 | −1.293 | −0.320 | −0.110 | 0.795 |
| DRD2-Tango | −0.397 | −0.101 | 1.657 | −1.851 | −1.222 | −0.411 | −0.726 | −0.871 |
| DRD3-Tango | −0.824 | −0.577 | 0.050 | −0.577 | −0.948 | −0.434 | 0.537 | 0.565 |
| DRD4-Tango | 0.068 | 0.313 | 1.133 | −0.824 | −0.846 | 0.685 | 1.270 | −0.667 |
| DRD5-Tango | −1.482 | −0.585 | −1.052 | −0.604 | −0.198 | −0.086 | −0.703 | 0.559 |
| EDNRA-Tango | −1.453 | −0.341 | −0.738 | −0.682 | −0.426 | −0.150 | −0.324 | 0.055 |
| EDNRB-Tango | −0.863 | −0.337 | −0.581 | −0.309 | −0.323 | 0.319 | −0.278 | −0.158 |
| ELTD1-Tango | −1.662 | −1.508 | −1.605 | −1.213 | −0.957 | −0.460 | −0.870 | −1.027 |
| F2R-Tango | −1.622 | −0.554 | −1.437 | −0.869 | −0.230 | −0.083 | −0.163 | 0.071 |
| F2RL1-Tango | −1.453 | −0.418 | −0.795 | −0.807 | −0.396 | −0.016 | 0.124 | −0.079 |
| F2RL2-Tango | −1.546 | 0.068 | −0.744 | −1.147 | −0.633 | 0.006 | 0.001 | −0.227 |
| F2RL3-Tango | −1.595 | −0.468 | −0.934 | −1.010 | −0.423 | −0.047 | −0.307 | −0.222 |
| FFA1-Tango | −0.952 | −0.510 | −0.639 | −1.237 | −0.662 | −0.300 | −0.393 | −0.444 |
| FFA2-Tango | −0.904 | −0.402 | −0.706 | −0.716 | −1.011 | 0.141 | −0.206 | −0.175 |
| FFA3-Tango | −1.012 | −0.014 | −0.168 | −0.881 | −0.581 | 0.058 | −0.011 | 0.096 |
| FPR1-Tango | −1.192 | −1.543 | −1.514 | −1.531 | −1.225 | −0.994 | −1.173 | −1.003 |
| FPR2-Tango | −0.760 | −0.243 | −0.156 | −0.407 | −0.396 | 0.212 | −0.518 | 0.051 |
| FPR3-Tango | −1.444 | −0.546 | −0.646 | −1.103 | −0.784 | −0.632 | −0.663 | −0.215 |
| FSHR-Tango | −1.001 | −0.256 | −0.406 | −0.636 | −0.498 | −0.149 | −0.577 | −0.423 |
| GABBR1-Tango | −0.483 | −0.238 | −0.601 | −0.189 | −0.341 | 0.110 | −0.371 | −0.308 |
| GAL1-Tango | −1.445 | −0.933 | −0.990 | −2.013 | −0.883 | −0.629 | −1.892 | −1.470 |
| GAL2-Tango | −1.824 | −0.497 | −1.070 | −0.761 | −0.079 | −0.753 | −1.350 | 0.791 |
| GAL3-Tango | −1.367 | −0.417 | −0.969 | −0.680 | −0.523 | −0.531 | −1.148 | −1.040 |
| GCGR-Tango | −1.473 | −0.458 | −0.771 | −0.493 | −0.505 | −0.242 | −1.136 | −0.376 |
| GHRHR-Tango | −0.990 | −0.422 | −0.442 | −1.014 | −0.782 | −0.505 | −0.465 | −0.671 |
| GHSR-Tango | −1.653 | −0.830 | −1.404 | −2.119 | −0.505 | −0.645 | −0.479 | −0.313 |
| GIPR-Tango | −1.742 | −1.021 | −1.238 | −1.203 | −0.416 | −0.200 | −0.740 | −0.122 |
| GLP1R-Tango | −1.757 | −0.402 | −0.864 | −1.190 | −0.423 | −0.514 | −1.059 | −0.283 |
| GLP2R-Tango | −0.755 | −0.052 | −0.247 | −1.477 | 0.023 | −0.835 | −0.496 | 0.080 |
| GNRHR-Tango | −0.518 | 0.264 | 0.000 | −0.832 | −0.209 | 0.062 | −0.542 | −0.377 |
| GPBA-Tango | −0.974 | −0.204 | −0.799 | −1.196 | −0.050 | −0.890 | −0.664 | −0.345 |
| GPER-Tango | −0.798 | −0.229 | −0.628 | −0.551 | −0.156 | −0.429 | −0.350 | −0.026 |
| GPR1-Tango | −1.148 | −0.261 | −0.493 | −0.923 | −0.755 | −0.555 | −0.372 | −0.487 |
| GPR101-Tango | −0.699 | −0.613 | −0.530 | −0.530 | −0.482 | −0.642 | −0.370 | −0.203 |
| GPR110-Tango | −1.108 | −0.554 | −0.685 | −1.059 | −0.482 | −0.549 | −0.699 | −0.634 |
| GPR111-Tango | −0.585 | −0.381 | −0.567 | −0.696 | −0.107 | −0.402 | −1.052 | −0.519 |
| GPR113-Tango | −1.051 | −0.621 | −0.792 | −0.544 | −0.735 | −0.511 | −0.540 | 0.092 |
| GPR114-Tango | −0.138 | 0.120 | −0.005 | −0.076 | −0.460 | 0.076 | −0.317 | −0.227 |
| GPR115-Tango | −0.892 | −0.458 | −0.386 | −0.651 | −0.786 | −0.356 | −0.588 | −0.812 |
| GPR116-Tango | −0.734 | −0.238 | −0.371 | −1.076 | −0.414 | −0.402 | −0.956 | −0.861 |
| GPR119-Tango | −0.279 | −0.108 | −0.261 | −0.679 | −0.549 | −0.189 | −0.277 | 0.125 |
| GPR12-Tango | −0.901 | −0.345 | −0.504 | −1.386 | −0.599 | −0.115 | −0.108 | −0.248 |
| GPR120-Tango | −0.190 | 0.045 | −0.072 | −0.436 | −0.296 | 0.200 | −0.214 | −0.086 |
| GPR123-Tango | −0.740 | 0.598 | 0.199 | −0.730 | −0.218 | −0.528 | −0.073 | 0.487 |
| GPR124-Tango | −0.718 | −0.081 | −0.134 | −1.772 | −1.050 | −0.036 | −0.678 | −0.184 |
| GPR125-Tango | −0.695 | −0.447 | −0.466 | −1.631 | −0.845 | −0.744 | −0.382 | −0.297 |
| GPR126-Tango | −0.943 | −0.531 | −0.753 | −1.358 | −0.898 | −0.352 | −1.000 | −0.603 |
| GPR132-Tango | −1.237 | −0.078 | −0.388 | −1.443 | −0.917 | −0.025 | −0.742 | −0.411 |
| GPR133-Tango | −1.073 | −0.218 | −0.343 | −1.196 | −0.815 | −0.200 | −0.953 | −0.617 |
| GPR135-Tango | −0.748 | −0.407 | −0.573 | −0.867 | −0.517 | −0.432 | −0.326 | 0.042 |
| GPR141-Tango | −0.641 | −0.061 | −0.182 | −1.680 | −0.439 | 0.164 | −0.793 | −0.402 |
| GPR142-Tango | −0.844 | 0.000 | −0.447 | −1.764 | −1.128 | −0.296 | −0.874 | −0.012 |
| GPR143-Tango | −1.436 | −0.187 | −0.328 | −0.992 | −0.717 | −0.619 | −0.410 | −0.329 |
| GPR144-Tango | −0.627 | −0.046 | −0.443 | −0.915 | −0.708 | −0.871 | −1.148 | −0.406 |
| GPR146-Tango | −0.471 | 0.846 | 0.451 | −0.835 | 0.000 | 0.415 | 0.982 | −0.217 |
| GPR148-Tango | −0.503 | −0.181 | −0.415 | −1.273 | 0.866 | −0.968 | 0.112 | 0.000 |
| GPR149-Tango | −1.331 | −0.703 | −1.102 | −1.617 | −1.541 | −1.033 | −1.138 | 0.060 |
| GPR15-Tango | −1.229 | 0.332 | −0.708 | −1.505 | −0.828 | −0.350 | −0.923 | 0.455 |
| GPR150-Tango | −0.648 | 0.200 | 0.000 | −1.307 | −0.270 | −0.255 | −0.087 | −0.255 |
| GPR151-Tango | −1.117 | 0.130 | −0.348 | −0.585 | −0.694 | −0.009 | −0.162 | −0.386 |
| GPR152-Tango | −0.556 | 0.184 | 0.144 | −0.656 | −0.607 | −0.193 | −0.613 | −0.542 |
| GPR153-Tango | −0.847 | −0.130 | −0.071 | −1.009 | −0.697 | −0.376 | −0.118 | −0.229 |
| GPR156-Tango | 0.021 | 0.166 | 0.329 | −1.012 | −0.547 | −1.307 | −1.071 | −0.367 |
| GPR157-Tango | −0.613 | 0.033 | −0.221 | −0.695 | −0.331 | −0.348 | −0.419 | −0.361 |
| GPR158-Tango | −0.419 | 0.021 | −0.141 | −0.832 | −0.258 | −0.206 | −0.833 | −0.720 |
| GPR160-Tango | −0.347 | 0.365 | 0.296 | −0.421 | −0.650 | 0.011 | −0.279 | −0.046 |
| GPR161-Tango | −0.466 | 0.074 | −0.236 | −0.899 | −0.467 | −0.304 | −0.135 | 0.060 |
| GPR162-Tango | −0.651 | −0.294 | −0.502 | −0.704 | −0.730 | −0.313 | −0.552 | −0.354 |
| GPR17-Tango | −0.213 | 0.164 | −0.060 | −0.731 | −0.902 | −0.557 | −0.116 | −0.050 |
| GPR171-Tango | 0.263 | 0.606 | 0.485 | −0.504 | −0.688 | 0.085 | −0.251 | −0.247 |
| GPR173-Tango | −0.932 | 0.454 | −0.162 | −0.751 | −0.316 | −0.462 | 0.084 | −0.098 |
| GPR174-Tango | −0.627 | −0.018 | −0.171 | −0.906 | −0.316 | 0.255 | −0.110 | 0.451 |
| GPR18-Tango | −0.830 | −0.108 | −0.443 | −0.738 | −0.861 | 0.408 | 0.058 | 0.674 |
| GPR182-Tango | −0.502 | 0.152 | −0.105 | −1.003 | −0.667 | −0.194 | 0.038 | −0.007 |
| GPR183-Tango | −0.556 | 0.045 | −0.217 | −0.480 | −0.357 | 0.275 | −0.194 | 0.018 |
| GPR19-Tango | −0.845 | 0.110 | −0.273 | −0.753 | −0.514 | 0.256 | −0.046 | −0.461 |
| GPR20-Tango | −0.240 | 0.026 | −0.335 | −0.553 | −0.204 | 1.216 | 0.170 | 0.104 |
| GPR21-Tango | −0.644 | 0.060 | −0.355 | −0.570 | −0.344 | −0.142 | −0.468 | 0.160 |
| GPR22-Tango | −0.636 | 0.141 | −0.046 | −0.802 | −0.146 | −0.095 | −0.286 | 0.064 |
| GPR25-Tango | −0.440 | 0.147 | −0.331 | −0.588 | 0.199 | 0.120 | −0.427 | −0.220 |
| GPR26-Tango | −1.145 | −0.642 | −0.657 | −1.700 | −0.318 | −0.164 | 0.436 | −0.564 |
| GPR27-Tango | −0.241 | 0.125 | 0.000 | −0.560 | 0.221 | 0.160 | −0.090 | 0.303 |
| GPR3-Tango | −0.724 | −0.359 | −0.384 | −1.363 | −0.779 | −0.243 | −0.507 | 0.105 |
| GPR31-Tango | −1.172 | −0.481 | −0.345 | −1.237 | −0.795 | 0.015 | −0.436 | −0.238 |
| GPR32-Tango | −1.449 | −0.834 | −0.958 | −0.798 | −0.128 | −0.684 | −1.154 | −0.080 |
| GPR34-Tango | −1.050 | −0.333 | −0.720 | −0.744 | −0.340 | −0.286 | −0.771 | 0.000 |
| GPR35-Tango | −1.138 | −0.229 | −0.666 | −1.689 | −0.815 | −0.476 | −0.759 | −0.147 |
| GPR37-Tango | −0.890 | −0.011 | −0.299 | −1.424 | −0.436 | −0.282 | −0.225 | 0.393 |
| GPR37L1-Tango | −0.572 | 0.017 | −0.117 | −1.042 | −0.636 | −0.240 | −0.186 | −0.249 |
| GPR39-Tango | −0.672 | −0.368 | −0.591 | −0.958 | −0.511 | −0.325 | −0.674 | −0.342 |
| GPR4-Tango | −0.873 | −0.251 | −0.448 | −1.189 | −0.777 | −0.370 | −0.292 | 0.180 |
| GPR44-Tango | −0.405 | −0.045 | −0.369 | −0.862 | −0.504 | −0.167 | −0.445 | −0.126 |
| GPR45-Tango | −1.108 | −1.048 | −0.672 | −1.181 | −0.124 | −0.554 | −0.756 | −0.106 |
| GPR50-Tango | −0.572 | 0.405 | 0.124 | −0.732 | −0.026 | −0.106 | 0.255 | 1.125 |
| GPR52-Tango | −0.589 | 0.144 | −0.097 | −1.097 | −0.118 | −0.386 | −0.048 | 0.103 |
| GPR55-Tango | −0.337 | 0.433 | 0.111 | −1.009 | −0.159 | −0.335 | 0.045 | −0.009 |
| GPR56-Tango | −1.125 | −0.033 | −0.187 | −1.623 | −0.351 | 0.309 | −0.016 | 0.116 |
| GPR6-Tango | −0.666 | 0.013 | −0.195 | −1.025 | −0.231 | 0.164 | 0.285 | 0.353 |
| GPR61-Tango | −0.942 | 0.596 | 0.378 | −0.985 | −0.493 | −0.131 | 0.043 | 0.478 |
| GPR62-Tango | −0.357 | 0.298 | 0.397 | −0.442 | −0.064 | 0.454 | −0.081 | −0.379 |
| GPR63-Tango | −0.450 | 0.080 | −0.185 | −0.338 | −0.747 | −0.552 | −0.598 | −0.696 |
| GPR64-Tango | −0.607 | −0.181 | −0.285 | −0.535 | −0.747 | −0.542 | −0.690 | −0.326 |
| GPR65-Tango | −0.791 | −0.170 | −0.353 | −0.437 | −0.353 | −0.551 | −0.369 | −0.240 |
| GPR68-Tango | −0.986 | −0.261 | −0.605 | −0.654 | −0.490 | −0.634 | −0.359 | −0.302 |
| GPR75-Tango | −0.665 | 0.336 | −0.093 | −0.494 | 0.106 | −0.270 | −0.056 | 0.492 |
| GPR77-Tango | −0.804 | −0.136 | −0.371 | −0.920 | −0.425 | −0.878 | −0.674 | −0.544 |
| GPR78-Tango | −0.924 | 0.212 | −0.444 | −0.725 | −0.241 | 0.170 | 0.172 | 0.005 |
| GPR82-Tango | −1.790 | −0.598 | −1.036 | −1.887 | −0.570 | −0.573 | −0.636 | 0.171 |
| GPR83-Tango | −0.821 | −0.002 | 0.007 | −1.394 | 0.142 | −0.303 | −0.109 | 0.262 |
| GPR84-Tango | −1.221 | −0.111 | −0.228 | −1.313 | −0.268 | −0.533 | −0.685 | 0.093 |
| GPR85-Tango | −0.094 | 0.150 | 0.228 | −0.586 | 0.136 | 0.016 | −0.541 | 0.145 |
| GPR87-Tango | −0.672 | 0.114 | 0.048 | −1.165 | 0.020 | −0.571 | −0.171 | 0.464 |
| GPR88-Tango | −0.396 | 0.084 | −0.135 | −0.358 | 0.236 | −0.192 | −0.179 | 0.386 |
| GPR97-Tango | −0.091 | 0.278 | 0.089 | −0.372 | −0.126 | 0.091 | −0.213 | −0.139 |
| GPRC5A-Tango | −1.108 | 0.002 | −0.344 | −1.420 | −0.433 | −0.290 | −0.272 | −0.219 |
| GPRC5B-Tango | −0.687 | 0.226 | −0.149 | −0.785 | −0.316 | 0.128 | −0.351 | 0.279 |
| GPRC5C-Tango | −0.788 | 0.446 | 0.331 | −0.288 | 0.356 | −0.029 | −0.396 | 0.394 |
| GPRC5D-Tango | −0.408 | 0.146 | 0.049 | −0.884 | −0.054 | −0.531 | −0.539 | −0.034 |
| GPRC6A-Tango | −0.614 | 0.123 | 0.018 | −1.103 | −0.198 | −0.364 | −0.937 | −0.227 |
| GRM1-Tango | −0.748 | −0.162 | −0.245 | −0.862 | −0.286 | −0.398 | −0.951 | −0.736 |
| GRM2-Tango | −0.400 | 0.160 | −0.270 | −0.588 | −0.122 | −0.158 | −0.778 | −0.407 |
| GRM4-Tango | −1.245 | 0.009 | 0.090 | −0.806 | −0.151 | 0.021 | 0.040 | −0.185 |
| GRM5-Tango | −0.730 | −0.186 | −0.325 | −1.140 | −0.177 | −0.280 | −0.726 | −0.714 |
| GRM6-Tango | −0.256 | −0.452 | −0.199 | −1.069 | −0.339 | −0.835 | −0.705 | −0.115 |
| GRM7-Tango | −1.240 | −0.881 | −0.527 | −2.117 | 0.029 | −1.209 | −1.571 | −0.470 |
| GRM8-Tango | −0.754 | −0.430 | −0.411 | −1.407 | −0.489 | −0.479 | −0.469 | −0.775 |
| GRPR-Tango | −0.849 | 0.110 | −0.270 | −1.394 | 0.312 | −0.301 | −0.156 | 0.658 |
| HCA1-Tango | −0.823 | 0.191 | −0.315 | −0.489 | 0.041 | 0.211 | −0.144 | 0.028 |
| HCA2-Tango | −1.140 | −0.114 | −0.077 | −0.930 | 0.210 | −0.654 | −0.404 | 0.233 |
| HCA3-Tango | −0.509 | 0.107 | −0.299 | −0.865 | −0.178 | 0.479 | −0.690 | 0.263 |
| HCTR1-Tango | −0.941 | −0.941 | −0.435 | −0.437 | 0.125 | 0.350 | 0.255 | −0.497 |
| HCTR2-Tango | −0.481 | 0.047 | −0.415 | −0.082 | −0.719 | −0.407 | −0.255 | 0.000 |
| HRH1-Tango | −1.408 | −0.709 | −0.955 | −1.530 | −0.359 | −0.840 | −0.246 | 0.312 |
| HRH2-Tango | 0.077 | 0.182 | 0.083 | −0.342 | 0.762 | 0.256 | −0.081 | 0.520 |
| HRH3-Tango | 0.000 | −0.132 | −0.170 | −0.523 | −0.176 | −0.913 | −0.442 | 0.156 |
| HRH4-Tango | −0.339 | −0.531 | −0.740 | −0.973 | −0.390 | −0.238 | 0.000 | 0.347 |
| HTR1A-Tango | −0.443 | −0.107 | −0.043 | −1.248 | 0.084 | 0.348 | 0.355 | 0.374 |
| HTR1B-Tango | −0.925 | −0.587 | −0.633 | −1.402 | −0.484 | −0.514 | −0.034 | 0.500 |
| HTR1D-Tango | −1.051 | −0.637 | −0.806 | −1.201 | −0.607 | −0.386 | −0.317 | 0.359 |
| HTR1E-Tango | −0.712 | −0.230 | −0.456 | −1.428 | −0.189 | −0.446 | 0.269 | 0.397 |
| HTR1F-Tango | −0.752 | −0.227 | −0.861 | −1.305 | −0.215 | −0.538 | −0.009 | 0.391 |
| HTR2A-Tango | −0.699 | −0.276 | −0.558 | −0.886 | −0.716 | −0.254 | −0.062 | 0.249 |
| HTR2B-Tango | −0.429 | −0.229 | −0.267 | −0.899 | −3.040 | −0.033 | −0.413 | 0.108 |
| HTR2C-Tango | −0.763 | −0.141 | −0.409 | −0.777 | −1.556 | −0.084 | −0.387 | −0.308 |
| HTR4-Tango | −0.952 | −0.061 | −0.546 | −0.180 | −1.314 | 0.232 | −0.091 | 0.595 |
| HTR5-Tango | −1.900 | −0.214 | −0.666 | −1.230 | −1.676 | −0.425 | 0.047 | 1.159 |
| HTR6-Tango | −0.635 | −0.118 | −0.363 | −0.942 | −0.575 | 0.151 | 0.011 | 0.111 |
| HTR7-Tango | 0.157 | 0.531 | 0.166 | −0.734 | −2.230 | −0.201 | 0.255 | 0.391 |
| KISSPEPTIN-R- | −0.792 | −0.207 | −0.833 | −0.436 | 0.335 | −0.219 | 0.326 | 0.122 |
| Tango | | | | | | | | |
| LHCGR-Tango | −1.064 | −0.401 | −0.078 | −0.932 | 0.230 | −0.218 | −0.310 | −0.540 |
| LPAR1-Tango | −0.938 | −0.325 | −1.669 | −1.210 | 0.061 | −0.252 | −0.407 | −0.241 |
| LPAR2-Tango | −1.075 | −0.230 | −0.961 | −0.892 | −0.007 | −0.292 | −0.680 | −0.427 |
| LPAR4-Tango | −1.394 | −0.710 | −1.799 | −1.434 | −0.153 | −0.463 | −0.393 | −0.119 |
| LPAR5-Tango | −0.509 | −0.082 | 0.128 | −0.835 | −0.225 | −0.119 | −0.289 | 0.043 |
| LPAR6-Tango | −0.915 | 0.192 | 0.873 | −0.917 | 0.387 | −0.192 | 0.085 | 0.470 |
| LTB4R-Tango | −0.507 | 0.299 | 0.358 | −0.576 | −0.089 | 0.076 | −0.076 | 0.045 |
| LTB4R2B-Tango | −1.488 | −0.478 | −0.539 | −0.639 | −0.117 | −0.024 | −0.158 | −0.078 |
| MAS1-Tango | −0.833 | −0.170 | −0.595 | −1.116 | −0.299 | −0.149 | −0.315 | −0.060 |
| MAS1L-Tango | −1.096 | −0.490 | −0.513 | −0.960 | 0.044 | −0.665 | −0.246 | −0.673 |
| MC1R-Tango | −0.267 | −0.012 | −0.281 | −0.331 | −0.012 | −0.161 | 0.009 | −0.146 |
| MC2R-Tango | −0.314 | 0.126 | −0.446 | 0.647 | 0.155 | 0.313 | 0.280 | 0.599 |
| MC3R-Tango | −0.974 | 0.423 | −0.109 | −0.669 | 0.499 | −0.326 | 0.547 | 0.637 |
| MC4R-Tango | −0.877 | 0.400 | −0.251 | −0.445 | 0.110 | 0.396 | 0.086 | 0.629 |
| MC5R-Tango | −0.191 | 0.083 | −0.395 | −1.766 | 0.178 | −0.471 | 0.226 | 0.514 |
| MCHR1-Tango | −0.674 | −0.511 | −0.523 | −1.488 | −1.331 | −1.369 | −0.696 | −0.461 |
| MCHR2-Tango | −0.262 | −0.199 | −0.320 | −1.075 | −0.493 | −0.390 | 0.102 | −0.234 |
| MLNR-Tango | 0.112 | 0.226 | −0.126 | −0.615 | −0.147 | −0.272 | −0.092 | 0.496 |
| MRGPRD-Tango | −1.014 | −0.325 | −0.629 | −1.503 | −0.843 | −0.690 | −0.195 | −0.286 |
| MRGPRE-Tango | 0.054 | 0.255 | 0.153 | −0.539 | −0.467 | −0.507 | −0.209 | 0.126 |
| MRGPRF-Tango | −0.358 | 0.144 | 0.087 | −0.812 | −0.040 | −0.526 | −0.212 | −0.216 |
| MRGPRG-Tango | −0.121 | −0.180 | 0.034 | −0.570 | −0.660 | −0.932 | −0.040 | −0.673 |
| MRGPRX1-Tango | −1.002 | −0.168 | −0.736 | −1.630 | −0.981 | −0.766 | 0.008 | −0.217 |
| MRGPRX2-Tango | −0.543 | −0.140 | −0.382 | −1.123 | −0.439 | −0.626 | −0.255 | −0.282 |
| MRGPRX3-Tango | −0.206 | −0.316 | 0.135 | −0.646 | −0.565 | 0.000 | −0.392 | −0.049 |
| MRGPRX4-Tango | −1.918 | −0.845 | −1.688 | −1.776 | −0.735 | −1.283 | −0.989 | −0.495 |
| MTNR1A-Tango | −1.563 | −0.377 | −1.299 | −1.323 | 0.232 | −0.742 | −0.643 | 0.205 |
| MTNR1B-Tango | −1.418 | −0.411 | −0.664 | −2.198 | −0.273 | −0.972 | −0.750 | 0.124 |
| NMBR-Tango | −0.385 | −0.054 | −0.228 | −0.237 | −0.011 | −0.274 | −0.355 | 0.490 |
| NMUR1-Tango | −0.893 | −0.749 | −0.861 | −1.130 | −0.044 | 0.821 | −1.386 | 0.082 |
| NMUR2-Tango | −0.984 | −0.045 | −0.433 | −1.916 | −0.118 | 0.627 | −1.040 | 0.628 |
| NPBW1-Tango | 0.097 | −0.128 | 0.235 | −0.923 | −0.798 | 0.376 | 0.480 | 0.133 |
| NPBW2-Tango | −1.265 | −0.893 | −0.514 | −1.797 | −0.841 | −1.129 | −0.599 | −1.352 |
| NPFF1-Tango | 0.293 | 0.519 | 0.235 | −0.490 | −0.124 | −1.128 | 0.387 | −0.662 |
| NPFF2-Tango | −0.720 | 0.249 | −0.262 | −0.608 | 0.337 | −0.414 | −0.146 | −0.015 |
| NPS-Tango | −0.738 | 0.096 | −0.125 | −1.195 | −0.112 | −0.865 | −0.045 | −0.215 |
| NPY1R-Tango | −1.101 | −0.192 | −0.504 | −0.544 | 0.089 | −0.623 | 0.072 | −0.426 |
| NPY2R-Tango | −0.579 | 0.477 | 0.523 | −0.423 | 0.364 | −0.163 | 0.069 | −0.190 |
| NPY4R-Tango | −0.250 | 0.013 | 0.000 | −0.643 | −0.163 | 0.651 | −0.577 | 0.493 |
| NPY5R-Tango | −0.644 | 0.453 | 0.091 | −0.851 | −0.033 | −0.100 | −0.450 | −0.141 |
| NTSR1-Tango | −1.176 | −0.459 | −0.904 | −1.128 | −0.472 | −0.529 | −0.808 | −0.189 |
| NTSR2-Tango | −1.130 | −0.577 | −1.055 | −0.280 | −0.043 | 0.156 | −0.241 | −0.308 |
| OPN3-Tango | −0.210 | 0.089 | −0.197 | −0.463 | −0.346 | −0.164 | 0.020 | 0.234 |
| OPN5-Tango | −1.856 | −0.571 | −1.120 | −1.272 | −0.513 | −0.874 | −0.839 | −0.458 |
| OPRD1-Tango | −1.097 | −0.610 | −0.670 | −1.089 | −0.889 | −0.628 | −0.855 | −0.055 |
| OPRK1-Tango | −1.039 | −0.116 | −0.272 | −1.036 | 0.280 | −0.218 | 0.236 | 0.157 |
| OPRL1-Tango | −0.704 | −0.108 | −0.220 | −0.662 | −0.125 | −0.436 | 0.065 | 0.909 |
| OPRM1-Tango | −0.704 | 0.170 | −0.730 | −1.032 | 0.490 | −0.488 | −0.626 | 0.027 |
| OXER1-Tango | −0.892 | −0.338 | −0.092 | −0.544 | 0.070 | −0.424 | −0.365 | −0.458 |
| OXGR1-Tango | −1.415 | −0.577 | −0.714 | −1.654 | −0.507 | −1.109 | −0.737 | −0.068 |
| OXTR-Tango | −0.376 | 0.090 | −0.261 | −0.705 | 0.040 | 0.144 | −0.535 | 0.181 |
| P2RY1-Tango | −1.311 | −0.490 | −0.832 | −1.657 | −0.480 | −0.430 | −1.018 | −0.184 |
| P2RY10-Tango | −0.882 | −0.279 | −0.392 | −0.773 | −0.070 | −0.222 | −0.421 | 0.112 |
| P2RY11-Tango | −1.192 | −0.738 | −0.674 | −0.818 | −0.478 | 0.090 | −0.710 | −0.550 |
| P2RY12-Tango | −1.497 | −0.179 | −0.320 | −0.726 | −0.533 | −0.643 | −1.517 | −0.385 |
| P2RY13-Tango | −0.763 | −0.138 | 0.040 | −1.340 | −0.727 | −0.561 | −0.478 | 0.155 |
| P2RY14-Tango | −0.932 | −0.156 | 0.126 | −1.009 | −1.195 | −0.787 | −0.382 | −0.227 |
| P2RY2-Tango | −1.224 | −0.401 | −0.343 | −1.165 | −0.734 | −0.430 | −0.710 | −0.402 |
| P2RY4-Tango | −1.325 | −0.703 | −0.623 | −1.881 | −1.035 | −0.524 | −0.739 | −0.169 |
| P2RY6-Tango | −0.945 | −0.395 | −0.364 | −1.345 | −1.136 | −0.692 | −0.739 | −0.164 |
| P2RY8-Tango | −1.092 | −0.147 | −0.373 | −1.250 | −1.245 | −0.832 | −0.625 | −0.056 |
| PK1-Tango | −0.579 | −0.013 | −0.183 | −0.744 | −0.635 | −0.162 | −0.043 | 0.118 |
| PK2-Tango | −0.436 | 0.183 | −0.354 | −0.354 | −0.619 | −0.374 | −0.285 | −0.346 |
| PRRP-Tango | −0.191 | 0.085 | 0.028 | −0.442 | −0.535 | −0.280 | −0.422 | 0.436 |
| PTAFR-Tango | −0.712 | −0.055 | 0.033 | −0.815 | −0.660 | −0.365 | −0.707 | 0.397 |
| PTGDR-Tango | −0.773 | −0.535 | −0.499 | −0.844 | −0.350 | −0.014 | −0.377 | −0.166 |
| PTGER1-Tango | −0.357 | −0.085 | −0.485 | −0.202 | −0.087 | 0.409 | 0.164 | −0.220 |
| PTGER2-Tango | −0.241 | −0.115 | −0.241 | −0.526 | −0.076 | −0.146 | −0.327 | 0.761 |
| PTGER3-Tango | −0.806 | −0.189 | −0.147 | −0.584 | −0.436 | −0.322 | −0.745 | 0.367 |
| PTGER4-Tango | −0.529 | 0.173 | −0.104 | −0.710 | −0.007 | 0.187 | 0.204 | 0.627 |
| PTGFR-Tango | −0.946 | −0.409 | −0.802 | −0.900 | −0.216 | 0.050 | −0.322 | 0.435 |
| PTGIR-Tango | −0.280 | −0.260 | −0.665 | −0.558 | −0.842 | 0.788 | −0.627 | 0.376 |
| PTH1R-Tango | −0.351 | 0.045 | 0.000 | −0.638 | −0.035 | 0.038 | −0.702 | 0.412 |
| PTH2R-Tango | −0.794 | −0.010 | −0.023 | −0.938 | −1.281 | −0.356 | −0.769 | 0.177 |
| QRFP-Tango | −0.446 | −0.085 | −0.200 | −0.815 | −0.427 | 0.152 | −0.758 | 0.073 |
| RXFP1-Tango | −1.146 | −0.786 | −0.599 | −0.997 | −0.759 | −0.406 | −0.693 | −0.407 |
| RXFP2-Tango | −1.309 | −0.275 | −0.324 | −1.204 | −1.177 | −0.447 | −0.651 | 0.047 |
| RXFP3-Tango | −0.434 | 0.076 | −0.260 | −1.213 | −0.498 | −0.333 | −1.138 | −0.153 |
| RXFP4-Tango | −0.503 | −0.478 | −0.125 | −0.391 | −0.346 | 0.527 | −0.888 | 0.260 |
| S1PR1-Tango | −0.776 | 0.141 | 0.089 | 0.452 | −0.781 | 0.077 | −0.416 | 0.074 |
| S1PR2-Tango | −1.680 | −0.374 | −0.508 | −0.585 | −0.785 | 0.230 | −0.036 | −0.031 |
| S1PR3-Tango | −1.172 | 0.308 | −0.057 | −0.190 | −0.320 | 0.601 | −0.170 | 0.253 |
| S1PR4-Tango | −1.560 | −0.400 | −0.636 | −0.280 | −1.012 | −1.147 | −0.813 | −0.274 |
| S1PR5-Tango | −0.159 | 0.547 | 0.089 | −0.094 | −0.320 | −0.087 | −0.523 | 0.057 |
| SCTR-Tango | −0.384 | −0.188 | −0.016 | 0.000 | 0.000 | −0.081 | 0.016 | 1.115 |
| SSTR1-Tango | −1.477 | −0.050 | −0.146 | −1.433 | −0.353 | −0.105 | −0.397 | 0.658 |
| SSTR2-Tango | −1.299 | −0.404 | −0.165 | −0.545 | −0.361 | −0.325 | −0.179 | −0.766 |
| SSTR3-Tango | −0.650 | 0.288 | −0.149 | −0.118 | 0.212 | 0.000 | 0.303 | 1.250 |
| SSTR4-Tango | −0.153 | 0.496 | 0.265 | −0.803 | −0.638 | −0.186 | 0.899 | 0.553 |
| SSTR5-Tango | 1.090 | 1.110 | 1.494 | −0.153 | −0.119 | 0.878 | 2.188 | 0.720 |
| SUCNR1-Tango | 3.693 | 3.615 | 3.868 | 1.245 | 2.167 | 2.407 | 3.913 | 2.771 |
| TA1-Tango | 0.380 | 0.537 | 0.651 | −0.195 | −0.878 | −0.133 | 0.125 | 0.763 |
| TAAR2-Tango | −0.794 | 0.016 | −0.118 | −0.321 | −0.345 | −0.032 | −0.394 | 0.596 |
| TAAR5-Tango | −0.163 | −0.138 | −0.739 | −0.957 | −0.503 | −1.001 | −0.351 | −0.538 |
| TAAR6-Tango | −0.391 | −0.252 | −3.777 | −1.014 | −0.878 | −0.931 | −0.243 | −0.475 |
| TAAR8-Tango | −0.447 | −0.105 | 0.106 | −0.774 | −0.586 | −0.714 | −0.580 | −0.727 |
| TAAR9-Tango | −0.807 | −0.117 | 0.000 | −0.978 | −0.609 | −0.636 | −0.556 | −0.532 |
| TACR1-Tango | −0.499 | −0.095 | 0.035 | −0.669 | −0.999 | −0.513 | −0.632 | 0.094 |
| TACR2-Tango | −0.393 | 0.063 | 0.123 | −0.327 | −0.471 | −0.450 | −0.376 | 0.145 |
| TACR3-Tango | −0.642 | 0.244 | −0.086 | −0.550 | −0.674 | −0.333 | −0.648 | 0.148 |
| TBXA2R-Tango | −0.387 | 0.160 | −0.100 | −0.753 | −0.650 | −0.177 | −0.423 | 0.257 |
| TSHR-Tango | −1.215 | −0.078 | 0.105 | −1.284 | −0.973 | −0.602 | −0.412 | −0.331 |
| UTS2R-Tango | 0.041 | 0.000 | 0.133 | −0.739 | −0.366 | 0.093 | −0.081 | −1.286 |
| VIPR1-Tango | −0.681 | −0.069 | −0.098 | −1.741 | −0.142 | −1.445 | −0.264 | −1.328 |
| VIPR2-Tango | 0.569 | 0.535 | 1.247 | 0.000 | 0.348 | −0.145 | 1.636 | 1.133 |

| PRESTO-Tango | Bacteroides | Bacteroides | Bacteroides | Bacteroides | Bacteroides | Bacteroides | Bacteroides | Bacteroides |
|---|---|---|---|---|---|---|---|---|
| with GMM | ovatus-1# | ovatus-2# | ovatus-3# | uniformis-1# | uniformis-2# | uniformis-3# | uniformis-4# | spp-1# |
| ADCYAP1R1- | −0.685 | −0.374 | 0.473 | −0.193 | −0.468 | −0.420 | −0.013 | −0.679 |
| Tango | | | | | | | | |
| ADORA1-Tango | −1.355 | −1.171 | −0.419 | −0.775 | −1.263 | −1.014 | −0.611 | −1.225 |
| ADORA2A-Tango | −0.441 | −0.472 | −0.229 | −0.322 | −0.693 | −0.817 | −0.631 | −0.034 |
| ADORA2B-Tango | −0.556 | −0.365 | −0.129 | −0.173 | −0.688 | −0.392 | −0.257 | −0.288 |
| ADORA3-Tango | −0.114 | −0.252 | 0.799 | 0.052 | −0.566 | −0.136 | 0.137 | −0.239 |
| ADRA1A-Tango | −0.419 | −0.632 | 0.157 | −0.225 | −0.539 | −0.654 | −0.351 | −0.315 |
| ADRA1B-Tango | −0.475 | −0.551 | 0.236 | −0.304 | −0.743 | −0.806 | −0.545 | −0.475 |
| ADRA1D-Tango | −0.412 | −0.676 | −0.114 | 0.288 | −0.222 | −0.718 | −1.361 | −0.615 |
| ADRA2A-Tango | −0.110 | −0.234 | 0.121 | −0.971 | −1.163 | 0.196 | −0.211 | −0.288 |
| ADRA2B-Tango | −1.034 | −1.063 | −0.222 | −1.303 | −1.500 | −1.672 | −1.226 | −1.391 |
| ADRA2C-Tango | 0.281 | −0.722 | 0.160 | −0.931 | −0.685 | −1.046 | −1.046 | 0.000 |
| ADRB1-Tango | −0.463 | −0.470 | 0.477 | −0.242 | −0.605 | −0.119 | −0.264 | −0.503 |
| ADRB2-Tango | −0.228 | −0.044 | 0.344 | 0.603 | −0.667 | −0.425 | −0.379 | −0.613 |
| ADRB3-Tango | −0.516 | −0.263 | −0.167 | −0.532 | −1.063 | −0.324 | 0.046 | −0.733 |
| AGTR1-Tango | −0.324 | 0.000 | 0.157 | −0.205 | −0.415 | −0.441 | −0.316 | −0.505 |
| AGTR2-Tango | −0.640 | −0.241 | 0.835 | 0.098 | −0.031 | −0.386 | −0.424 | −0.519 |
| APJ-Tango | −0.241 | −0.989 | 0.449 | 0.175 | −0.238 | −0.280 | −0.381 | −0.229 |
| AVPR1A-Tango | −0.059 | −0.138 | 0.091 | −0.140 | −0.520 | −0.128 | 0.180 | −0.507 |
| AVPR1B-Tango | −0.642 | −0.179 | 0.298 | −0.639 | −0.716 | −0.478 | −0.098 | −0.493 |
| AVPR2-Tango | −0.586 | −0.945 | 0.040 | −1.100 | −1.270 | −0.189 | −0.285 | −0.687 |
| BB3-Tango | −0.528 | −0.676 | −0.049 | −1.381 | −1.605 | −0.807 | −0.835 | −0.836 |
| BDKBR1-Tango | −0.733 | −0.420 | 0.480 | −0.835 | −1.007 | −0.234 | −0.229 | −0.219 |
| BDKBR2-Tango | −0.442 | −0.095 | 0.769 | 0.341 | −0.356 | 0.403 | 0.517 | −0.477 |
| C3AR1-Tango | −1.002 | −0.575 | −0.192 | −0.649 | −0.939 | −0.218 | 0.154 | −0.793 |
| C5A-Tango | −0.262 | −0.084 | 0.795 | 0.494 | −0.586 | −0.409 | −0.182 | −0.025 |
| CALCRb-Tango | −0.119 | −0.133 | −0.010 | 0.070 | −0.745 | −0.348 | −0.325 | −0.092 |
| CALCRL-Tango | 0.135 | −0.189 | −0.190 | 0.090 | −0.245 | −0.117 | −0.132 | 0.018 |
| CASR-Tango | −0.413 | 0.082 | 0.433 | −0.400 | −0.854 | −1.165 | −0.518 | −0.596 |
| CCKAR-Tango | −0.267 | 0.132 | 0.468 | −0.070 | −0.620 | −0.335 | 0.048 | −0.319 |
| CCKBR-Tango | −0.445 | −0.788 | 0.072 | −0.387 | −0.779 | −0.527 | −0.518 | −0.496 |
| CCR10-Tango | −0.616 | −0.382 | −0.162 | −0.392 | −0.786 | −0.724 | −0.284 | −0.332 |
| CCR2-Tango | 0.049 | −0.259 | 0.101 | −0.171 | −0.252 | −0.468 | −0.312 | −0.231 |
| CCR3-Tango | −1.074 | −0.635 | 0.109 | 0.377 | −0.029 | −0.649 | −0.443 | −0.786 |
| CCR4-Tango | −0.615 | −0.197 | 0.805 | 0.094 | −0.398 | −1.014 | −0.540 | −0.096 |
| CCR5-Tango | −0.148 | −0.013 | 0.193 | −0.106 | −0.660 | −0.504 | −0.270 | −0.031 |
| CCR6-Tango | −0.654 | 0.010 | 0.221 | 0.812 | 0.256 | −0.190 | −0.090 | −0.456 |
| CCR7-Tango | −0.353 | −0.221 | 0.000 | −0.125 | −0.771 | −0.504 | −0.125 | −0.159 |
| CCR8-Tango | −0.315 | −0.350 | 0.429 | 0.086 | −0.429 | −0.842 | −0.401 | −0.058 |
| CCRL2-Tango | −0.387 | −0.197 | −0.393 | −0.327 | −0.585 | −0.318 | −0.631 | −0.173 |
| CD97-Tango | −0.745 | −0.939 | 0.290 | −0.886 | −0.886 | −0.968 | −0.752 | −0.601 |
| CHRM1-Tango | −0.120 | −0.564 | 0.113 | −0.896 | −0.645 | −0.160 | −0.482 | 0.192 |
| CHRM2-Tango | −0.610 | −0.944 | 0.295 | −0.793 | −1.256 | −0.353 | −0.169 | −0.435 |
| CHRM3-Tango | −0.230 | −0.778 | 0.107 | −0.598 | −1.115 | −0.939 | −0.800 | −0.516 |
| CHRM4-Tango | −0.293 | 0.047 | 0.285 | −0.425 | −0.572 | −0.609 | −0.261 | −0.382 |
| CHRM5-Tango | −0.304 | 0.260 | 0.634 | −0.365 | −0.636 | −0.149 | −0.211 | −0.254 |
| CMKLR1-Tango | −0.131 | −0.074 | 0.403 | −0.085 | −0.444 | −0.232 | 0.194 | −0.224 |
| CNR1-Tango | −0.471 | −0.316 | 0.709 | −0.339 | −0.821 | −0.751 | −0.213 | −0.128 |
| CNR2-Tango | −0.526 | −0.436 | 0.295 | −0.392 | −0.803 | −0.405 | −0.027 | −0.549 |
| CRHR1-Tango | 0.000 | −0.878 | −0.234 | −0.127 | −0.127 | −0.165 | −0.335 | −0.075 |
| CRHR2-Tango | −0.729 | −0.503 | 0.538 | −0.599 | −0.371 | −0.100 | −0.049 | −0.234 |
| CX3C1-Tango | −0.792 | −0.712 | 0.552 | −0.334 | −0.298 | 0.091 | 0.037 | −0.740 |
| CXCR1-Tango | −0.160 | 0.080 | 0.455 | −0.985 | −0.675 | −0.402 | −0.246 | 0.174 |
| CXCR2-Tango | −0.661 | −0.149 | 0.429 | −0.276 | −0.309 | −0.706 | −0.462 | −0.493 |
| CXCR3-Tango | −0.306 | −0.545 | 0.337 | −0.208 | −0.252 | −0.087 | −0.072 | −0.318 |
| CXCR4-Tango | −0.342 | −0.083 | 0.332 | −0.251 | −0.298 | −0.133 | −0.057 | −0.338 |
| CXCR5-Tango | −0.745 | −0.254 | 0.247 | 0.031 | −0.468 | −0.271 | −0.129 | −0.525 |
| CXCR6-Tango | −0.434 | −0.371 | 0.523 | −0.457 | −0.627 | −0.144 | 0.142 | −0.721 |
| CXCR7-Tango | −1.257 | −1.182 | −0.061 | −1.029 | −1.144 | −1.119 | −0.775 | −1.011 |
| CYSLTR1-Tango | −0.807 | −0.383 | −0.091 | −0.380 | −0.487 | −0.756 | −0.787 | −0.312 |
| CYSLTR2-Tango | 0.000 | −0.405 | 0.416 | −0.441 | −0.463 | −0.518 | −0.568 | −0.075 |
| DRD1-Tango | −0.174 | −0.263 | 0.272 | −0.426 | −0.838 | −0.710 | −0.093 | 0.048 |
| DRD2-Tango | −0.823 | −0.419 | 0.412 | −1.616 | −2.234 | −1.672 | −1.029 | −1.010 |
| DRD3-Tango | 0.678 | 0.787 | 0.769 | −2.481 | −2.916 | −1.449 | −1.554 | 0.485 |
| DRD4-Tango | −1.322 | 0.404 | 0.126 | −1.835 | −2.867 | −1.799 | −1.174 | −0.824 |
| DRD5-Tango | −0.478 | −0.724 | −0.111 | −1.000 | −0.715 | −0.372 | −0.189 | −0.263 |
| EDNRA-Tango | −0.422 | −0.340 | 0.541 | −0.035 | −0.606 | −0.046 | 0.237 | −0.385 |
| EDNRB-Tango | −0.180 | −0.436 | 0.238 | −0.670 | −0.538 | −0.238 | −0.020 | −0.167 |
| ELTD1-Tango | −0.722 | −0.657 | 0.140 | −0.497 | −0.885 | −0.952 | −0.699 | −0.819 |
| F2R-Tango | −0.503 | −0.138 | 0.370 | 0.042 | −0.428 | −0.361 | 0.065 | −0.540 |
| F2RL1-Tango | −0.448 | 0.249 | 0.682 | 0.064 | −0.636 | −0.319 | −0.019 | −0.296 |
| F2RL2-Tango | −0.682 | 0.208 | 0.341 | −0.474 | −0.989 | −0.494 | −0.311 | −0.702 |
| F2RL3-Tango | −0.525 | −0.284 | 0.336 | −0.431 | −0.718 | −0.358 | −0.200 | −0.788 |
| FFA1-Tango | −0.520 | −0.264 | 0.441 | −0.147 | −0.273 | 0.016 | −0.075 | −0.504 |
| FFA2-Tango | −0.228 | −0.463 | 0.175 | −0.581 | −0.697 | −0.321 | −0.398 | −0.615 |
| FFA3-Tango | −0.345 | 0.251 | 0.607 | 0.272 | −0.721 | −0.345 | 0.123 | −0.234 |
| FPR1-Tango | −1.098 | −0.567 | 0.070 | −1.559 | −1.536 | −2.035 | −1.119 | −1.422 |
| FPR2-Tango | −0.372 | −0.500 | 0.280 | −0.709 | −1.143 | −0.289 | 0.022 | −0.055 |
| FPR3-Tango | −0.462 | −0.383 | 0.313 | −0.576 | −0.677 | −0.154 | −0.191 | −0.471 |
| FSHR-Tango | −0.257 | −0.437 | 0.314 | −0.810 | −0.850 | −0.286 | −0.199 | −0.279 |
| GABBR1-Tango | −0.336 | −0.242 | 0.124 | −1.106 | −0.863 | −0.775 | −0.243 | −0.140 |
| GAL1-Tango | −0.733 | −1.080 | 0.347 | −0.819 | −1.085 | −0.836 | −0.449 | −1.328 |
| GAL2-Tango | −0.031 | −0.719 | 0.357 | −0.209 | −1.040 | −0.381 | −0.171 | −0.534 |
| GAL3-Tango | −0.827 | −1.101 | 0.133 | −0.385 | −1.209 | −0.836 | 0.075 | −0.569 |
| GCGR-Tango | −0.189 | −1.263 | 0.140 | −0.878 | −1.133 | −0.645 | −0.112 | −0.289 |
| GHRHR-Tango | −0.492 | −0.022 | 0.656 | −0.457 | −0.786 | −0.135 | 0.198 | −0.423 |
| GHSR-Tango | −1.211 | −0.386 | 0.239 | −0.628 | −0.930 | −0.212 | −0.105 | −1.295 |
| GIPR-Tango | −0.728 | −0.177 | −0.247 | −0.574 | −1.272 | −0.214 | 0.000 | −0.708 |
| GLP1R-Tango | −0.557 | −0.491 | 0.246 | −0.081 | −1.289 | −0.472 | 0.291 | −0.863 |
| GLP2R-Tango | −0.041 | −0.609 | 0.369 | 0.432 | −0.216 | −0.068 | 0.192 | −0.189 |
| GNRHR-Tango | −0.160 | −0.075 | −0.114 | −0.265 | −0.988 | −0.148 | −0.191 | 0.018 |
| GPBA-Tango | −0.395 | −1.122 | 0.242 | −0.585 | −0.628 | −0.328 | −0.822 | −0.595 |
| GPER-Tango | −0.647 | 0.033 | 0.412 | −0.618 | −0.746 | −0.408 | −0.119 | −0.760 |
| GPR1-Tango | −0.484 | −0.527 | 0.118 | −0.682 | −1.545 | −0.374 | −0.579 | −0.387 |
| GPR101-Tango | −0.222 | −0.071 | 0.592 | −0.662 | −0.918 | −0.047 | −0.019 | −0.246 |
| GPR110-Tango | −0.839 | −0.718 | 0.103 | −1.007 | −0.897 | −0.707 | −0.667 | −0.887 |
| GPR111-Tango | −0.711 | −0.073 | 0.076 | −1.015 | −1.100 | −0.260 | −0.135 | −0.430 |
| GPR113-Tango | −0.024 | −0.540 | −0.109 | −0.292 | −0.758 | −0.461 | 0.110 | −0.460 |
| GPR114-Tango | −0.069 | −0.511 | −0.071 | −0.423 | −0.941 | 0.000 | −0.244 | 0.147 |
| GPR115-Tango | −0.637 | −0.603 | −0.063 | −0.254 | −0.717 | −0.354 | −0.375 | −0.774 |
| GPR116-Tango | −0.800 | −0.875 | −0.163 | −0.529 | −0.780 | −0.716 | −0.544 | −1.042 |
| GPR119-Tango | −0.376 | −0.319 | −0.269 | −0.373 | −0.610 | −0.157 | −0.181 | −0.201 |
| GPR12-Tango | −0.706 | −0.515 | 0.528 | −0.558 | −0.496 | −0.070 | 0.023 | −0.537 |
| GPR120-Tango | −0.177 | −0.271 | 0.160 | −0.622 | −0.767 | −0.634 | −0.159 | −0.292 |
| GPR123-Tango | −0.350 | −0.315 | 0.250 | −0.432 | −0.517 | −0.332 | 0.052 | −0.232 |
| GPR124-Tango | −0.861 | −0.467 | 0.046 | −0.340 | −0.653 | −0.724 | −0.715 | −0.785 |
| GPR125-Tango | −1.163 | −0.099 | −0.130 | −0.317 | −0.497 | −0.791 | −0.824 | −0.900 |
| GPR126-Tango | −0.795 | −0.757 | −0.191 | −0.805 | −0.958 | −0.133 | −0.365 | −0.767 |
| GPR132-Tango | −0.813 | −0.870 | −0.048 | −0.526 | −0.795 | −0.759 | −0.553 | −0.931 |
| GPR133-Tango | −0.579 | −0.818 | −0.318 | −0.436 | −0.668 | −0.551 | −0.419 | −0.811 |
| GPR135-Tango | −0.488 | −0.452 | 0.006 | −0.241 | −0.628 | 0.000 | 0.009 | −0.745 |
| GPR141-Tango | −0.981 | −0.691 | 0.720 | −0.276 | −0.619 | −0.393 | −0.451 | −0.925 |
| GPR142-Tango | −0.887 | −0.899 | 0.140 | 0.202 | −0.646 | −0.790 | −0.696 | −1.055 |
| GPR143-Tango | −0.453 | −0.126 | 0.592 | 0.041 | −1.160 | −0.930 | −0.848 | −0.444 |
| GPR144-Tango | −0.605 | −0.698 | −0.102 | −0.666 | −1.387 | −0.745 | −0.870 | −0.680 |
| GPR146-Tango | −0.358 | 0.000 | 0.358 | −0.208 | −1.270 | 0.068 | −0.121 | −0.204 |
| GPR148-Tango | −0.951 | −0.122 | −0.181 | −1.511 | −1.132 | −0.828 | −0.723 | −1.025 |
| GPR149-Tango | −0.114 | −0.050 | 0.334 | −1.314 | −1.257 | −0.551 | −0.855 | −0.851 |
| GPR15-Tango | −0.120 | −0.150 | 0.137 | −0.229 | −1.186 | −0.052 | 0.000 | −0.494 |
| GPR150-Tango | −0.420 | −0.585 | 0.491 | −0.531 | −0.531 | −0.093 | −0.093 | −0.220 |
| GPR151-Tango | −0.230 | −0.019 | 0.730 | −0.218 | −0.857 | −0.086 | −0.086 | −0.708 |
| GPR152-Tango | −0.166 | −0.376 | 0.295 | −0.061 | −0.420 | −0.560 | −0.370 | −0.301 |
| GPR153-Tango | −0.475 | −0.140 | 0.324 | −0.559 | −0.699 | −0.381 | −0.225 | −0.532 |
| GPR156-Tango | −0.480 | −0.963 | 0.161 | −0.413 | −0.687 | −0.127 | −0.291 | −0.571 |
| GPR157-Tango | −0.477 | −0.145 | 0.201 | 0.053 | −0.253 | −0.556 | −0.329 | −0.214 |
| GPR158-Tango | 0.023 | −0.987 | 0.080 | −0.312 | −0.606 | −0.324 | −0.108 | −0.284 |
| GPR160-Tango | −0.176 | −0.189 | 0.343 | 0.057 | −0.581 | −0.220 | −0.202 | −0.137 |
| GPR161-Tango | −0.483 | 0.026 | 0.356 | −0.486 | −0.651 | −0.476 | −0.568 | −0.430 |
| GPR162-Tango | −0.456 | −0.376 | −0.072 | −0.811 | −1.017 | −0.562 | −0.466 | −0.274 |
| GPR17-Tango | −0.335 | −0.005 | 0.438 | −0.036 | −0.129 | −0.697 | −0.786 | −0.472 |
| GPR171-Tango | −0.409 | −0.215 | 0.415 | −0.118 | −0.550 | −0.704 | −0.797 | −0.172 |
| GPR173-Tango | −0.400 | 0.084 | 0.138 | −0.115 | −0.720 | −0.564 | −0.795 | −0.462 |
| GPR174-Tango | −0.328 | −0.157 | 0.465 | −0.327 | −0.257 | −0.490 | −0.536 | −0.301 |
| GPR18-Tango | −0.498 | 0.144 | 0.552 | 0.113 | −0.409 | −0.748 | −0.362 | −0.197 |
| GPR182-Tango | −0.471 | 0.061 | 0.134 | −0.437 | −0.482 | −0.762 | −0.676 | −0.509 |
| GPR183-Tango | −0.404 | −0.254 | 0.446 | −0.014 | −0.231 | −0.197 | −0.138 | −0.352 |
| GPR19-Tango | −0.379 | 0.078 | 0.280 | 0.103 | −0.489 | −0.537 | −0.352 | −0.559 |
| GPR20-Tango | −0.191 | 0.700 | 0.000 | −0.234 | −0.415 | −0.095 | 0.240 | −0.463 |
| GPR21-Tango | 0.103 | −0.515 | −0.017 | −0.459 | −0.830 | −0.175 | −0.078 | −0.312 |
| GPR22-Tango | −0.383 | −0.005 | 0.527 | 0.017 | −0.545 | −0.263 | −0.266 | −0.277 |
| GPR25-Tango | −0.353 | 0.000 | 0.700 | −0.484 | −0.461 | −0.404 | −0.229 | −0.060 |
| GPR26-Tango | −0.829 | 0.170 | 0.242 | −0.064 | −0.260 | −0.066 | −0.440 | −1.003 |
| GPR27-Tango | 0.017 | −0.187 | 0.709 | −0.511 | −0.435 | −0.244 | −0.200 | 0.034 |
| GPR3-Tango | −0.596 | −0.447 | 0.794 | −0.284 | −0.342 | 0.212 | 0.056 | −0.410 |
| GPR31-Tango | −0.979 | −0.218 | 0.198 | −0.425 | −0.639 | −0.315 | 0.018 | −0.695 |
| GPR32-Tango | −0.503 | −0.565 | −0.184 | −0.752 | −0.426 | −0.314 | −0.248 | −0.108 |
| GPR34-Tango | −0.798 | −0.783 | −0.011 | −0.775 | −1.040 | −0.189 | −0.016 | −0.684 |
| GPR35-Tango | −0.812 | −0.987 | 0.289 | −0.761 | −0.895 | −0.244 | −0.225 | −0.826 |
| GPR37-Tango | −0.936 | −0.508 | 0.389 | −0.962 | −1.204 | −0.264 | −0.035 | −0.845 |
| GPR37L1-Tango | −0.534 | −0.385 | 1.152 | −0.180 | −0.536 | −0.194 | −0.077 | −0.688 |
| GPR39-Tango | −0.649 | −0.736 | 0.558 | −0.396 | −0.466 | −0.044 | −0.248 | −0.679 |
| GPR4-Tango | −0.781 | −0.249 | 0.429 | −0.799 | −0.826 | 0.003 | −0.200 | −0.813 |
| GPR44-Tango | −0.433 | −0.669 | 0.120 | −0.637 | −0.573 | −0.136 | 0.000 | −0.319 |
| GPR45-Tango | −0.315 | −0.816 | −0.194 | −0.148 | −0.335 | −0.261 | −0.097 | −0.315 |
| GPR50-Tango | −0.567 | 0.378 | 0.391 | 0.238 | −0.577 | 0.114 | −0.216 | −0.070 |
| GPR52-Tango | −0.454 | 0.013 | 0.619 | −0.556 | −1.358 | 0.103 | −0.024 | −0.345 |
| GPR55-Tango | −0.276 | −0.134 | 0.281 | −0.864 | −1.792 | 0.069 | −0.043 | −0.622 |
| GPR56-Tango | −0.375 | 0.606 | 0.907 | 0.149 | −0.960 | 0.115 | −0.108 | 0.152 |
| GPR6-Tango | −0.487 | −0.071 | 0.456 | −0.183 | −0.512 | −0.294 | −0.099 | −0.362 |
| GPR61-Tango | −0.402 | 0.070 | 0.208 | −0.180 | −0.903 | 0.013 | −0.040 | −0.414 |
| GPR62-Tango | −0.539 | 0.000 | −0.491 | 0.079 | −0.330 | 0.158 | 0.133 | −0.259 |
| GPR63-Tango | −0.046 | −0.700 | 0.328 | −0.480 | −0.961 | −0.757 | −0.642 | −0.238 |
| GPR64-Tango | −0.250 | −0.573 | −0.118 | −1.095 | −1.317 | −0.330 | −0.255 | −0.372 |
| GPR65-Tango | −0.126 | −0.266 | 0.149 | −0.625 | −0.790 | −0.026 | −0.140 | −0.132 |
| GPR68-Tango | −0.617 | −0.264 | −0.165 | −0.860 | −1.123 | −0.351 | 0.026 | −0.627 |
| GPR75-Tango | −0.055 | −0.027 | 0.200 | −0.196 | −0.889 | −0.265 | −0.142 | 0.114 |
| GPR77-Tango | −0.485 | −0.591 | 0.087 | −0.780 | −1.437 | −0.387 | −0.227 | −0.406 |
| GPR78-Tango | −0.006 | 0.298 | 0.495 | −0.078 | −0.822 | −0.190 | −0.116 | 0.174 |
| GPR82-Tango | −0.783 | −0.095 | 0.148 | −0.147 | −0.703 | −0.204 | −0.242 | −0.600 |
| GPR83-Tango | −0.600 | −0.228 | 0.529 | −0.670 | −0.826 | −0.532 | −0.246 | −0.686 |
| GPR84-Tango | −0.597 | −0.485 | 0.497 | −0.409 | −0.936 | −0.526 | −0.318 | −0.569 |
| GPR85-Tango | −0.274 | −0.379 | 0.224 | −0.339 | −0.870 | −0.396 | −0.316 | −0.284 |
| GPR87-Tango | −0.460 | −0.258 | 0.313 | −0.259 | −0.389 | −0.224 | −0.254 | −0.442 |
| GPR88-Tango | 0.112 | −0.120 | 0.350 | −0.651 | −0.662 | −0.263 | −0.342 | −0.242 |
| GPR97-Tango | −0.054 | −0.253 | −0.044 | −0.681 | −1.005 | −0.550 | −0.740 | −0.120 |
| GPRC5A-Tango | −0.659 | −0.158 | 0.182 | −0.247 | −0.907 | −0.163 | −0.277 | −0.686 |
| GPRC5B-Tango | −0.374 | −0.314 | 0.612 | −0.083 | −0.709 | −0.511 | −0.468 | −0.281 |
| GPRC5C-Tango | −0.182 | −0.319 | 0.708 | 0.054 | −0.366 | −0.381 | −0.387 | 0.112 |
| GPRC5D-Tango | −0.314 | −0.606 | 0.167 | −0.569 | −0.829 | −0.553 | −0.456 | −0.443 |
| GPRC6A-Tango | −0.535 | −0.943 | 0.353 | −0.659 | −0.729 | −0.531 | −0.302 | −0.663 |
| GRM1-Tango | −0.706 | −0.772 | −0.079 | −0.288 | −0.731 | −0.653 | −0.375 | −0.520 |
| GRM2-Tango | −0.313 | −0.534 | −0.049 | −0.868 | −1.341 | −0.705 | −0.533 | −0.294 |
| GRM4-Tango | −0.361 | −0.022 | 0.450 | −0.783 | −1.410 | −0.543 | −0.241 | −0.433 |
| GRM5-Tango | −0.507 | −0.782 | −0.010 | −1.035 | −1.035 | −0.305 | −0.815 | −0.638 |
| GRM6-Tango | −0.793 | −0.769 | 0.277 | −1.224 | −0.806 | −0.320 | −0.391 | −0.811 |
| GRM7-Tango | −0.744 | −1.346 | −0.119 | −0.968 | −1.321 | −0.447 | −0.741 | −0.793 |
| GRM8-Tango | −0.773 | −0.435 | 0.292 | −0.762 | −0.990 | −0.966 | −0.749 | −0.590 |
| GRPR-Tango | −0.414 | 0.079 | 0.329 | −0.615 | −0.322 | −0.331 | −0.212 | −0.431 |
| HCA1-Tango | −0.363 | 0.515 | 0.485 | 0.242 | −0.327 | −0.269 | −0.515 | −0.344 |
| HCA2-Tango | −0.505 | −0.581 | 0.592 | −0.223 | −0.846 | −0.914 | −0.840 | −0.216 |
| HCA3-Tango | −0.473 | −0.442 | 0.557 | 0.280 | −0.322 | −0.785 | −0.874 | −0.585 |
| HCTR1-Tango | −0.553 | 0.675 | 0.626 | 0.585 | −0.280 | −0.160 | 0.434 | −0.553 |
| HCTR2-Tango | 0.152 | 0.282 | 0.310 | −0.112 | −0.368 | −0.497 | −0.497 | 0.152 |
| HRH1-Tango | −0.533 | −0.225 | 0.648 | −0.625 | −0.739 | −1.182 | −0.283 | −0.667 |
| HRH2-Tango | −0.187 | 0.018 | 0.554 | −0.329 | −0.443 | −0.558 | −0.345 | −0.282 |
| HRH3-Tango | −0.399 | −0.380 | 0.645 | −0.615 | −0.615 | −1.324 | −1.039 | −0.213 |
| HRH4-Tango | −0.697 | −0.534 | 0.423 | −0.750 | −0.262 | −0.468 | −0.468 | −0.697 |
| HTR1A-Tango | −0.404 | 0.094 | 0.500 | −0.321 | −0.561 | −0.517 | −0.620 | −0.367 |
| HTR1B-Tango | −0.835 | −0.189 | 0.611 | −0.716 | −0.686 | −0.524 | −0.464 | −0.807 |
| HTR1D-Tango | −0.456 | −0.418 | 0.476 | −0.486 | −0.470 | −0.227 | −0.364 | −0.401 |
| HTR1E-Tango | −0.660 | 0.122 | 0.572 | −0.359 | −0.509 | −0.518 | −0.350 | −0.815 |
| HTR1F-Tango | −0.510 | 0.029 | −0.088 | −0.398 | −0.577 | −1.011 | −0.398 | −0.542 |
| HTR2A-Tango | −0.341 | −0.224 | 0.168 | −0.723 | −0.832 | −1.272 | −0.861 | −0.398 |
| HTR2B-Tango | −0.442 | −0.528 | 0.240 | −0.959 | −1.113 | −0.625 | −0.546 | −0.474 |
| HTR2C-Tango | −0.265 | −0.203 | 0.172 | −0.671 | −0.933 | −0.662 | −0.924 | −0.255 |
| HTR4-Tango | 0.014 | 0.194 | 0.591 | −0.475 | −0.820 | −0.177 | −0.081 | 0.302 |
| HTR5-Tango | −0.669 | 0.221 | 0.384 | 0.000 | −0.655 | −0.038 | 0.195 | −0.620 |
| HTR6-Tango | −0.597 | −0.136 | 0.464 | −0.268 | −1.000 | −0.736 | −0.798 | −0.401 |
| HTR7-Tango | −0.662 | 0.055 | −0.193 | −0.699 | −1.058 | −0.204 | −0.499 | −0.433 |
| KISSPEPTIN-R- | −0.094 | −0.154 | 0.075 | −0.068 | −0.531 | 0.263 | −0.282 | 0.108 |
| Tango | | | | | | | | |
| LHCGR-Tango | −0.555 | −0.202 | 0.547 | 0.054 | −0.183 | −1.304 | −0.702 | −0.828 |
| LPAR1-Tango | −0.227 | −0.289 | 0.840 | −0.363 | −0.985 | −0.518 | −0.225 | −0.787 |
| LPAR2-Tango | −0.542 | −0.451 | 0.535 | −0.149 | −0.814 | −0.681 | −0.588 | −0.671 |
| LPAR4-Tango | −0.838 | −0.261 | 0.406 | −0.409 | −0.551 | −0.870 | −1.079 | −0.718 |
| LPAR5-Tango | −0.670 | −0.349 | 0.422 | −0.599 | −0.578 | −1.000 | −1.336 | −0.356 |
| LPAR6-Tango | −0.594 | 0.281 | 0.516 | 0.037 | −0.407 | −1.250 | −0.864 | −0.395 |
| LTB4R-Tango | −0.194 | 0.097 | 0.426 | 0.180 | −0.588 | −0.520 | −0.482 | −0.239 |
| LTB4R2B-Tango | −0.371 | −0.255 | 0.672 | 0.096 | −0.569 | −0.766 | −0.444 | −0.490 |
| MAS1-Tango | −0.463 | −0.211 | 0.454 | −0.639 | −0.649 | −0.274 | −0.568 | −0.311 |
| MAS1L-Tango | −0.486 | −0.207 | 0.504 | −0.728 | −1.190 | −0.703 | −0.517 | −0.398 |
| MC1R-Tango | −0.149 | −0.183 | 0.266 | −0.810 | −0.670 | −0.352 | −0.466 | −0.043 |
| MC2R-Tango | 0.715 | 0.147 | 0.569 | −0.585 | −0.982 | 0.144 | −0.410 | 0.160 |
| MC3R-Tango | 0.256 | 0.529 | 1.348 | 0.516 | −0.560 | −1.064 | −0.047 | 0.223 |
| MC4R-Tango | −0.445 | 0.230 | 0.741 | 0.225 | −0.229 | −0.031 | −0.660 | −0.286 |
| MC5R-Tango | −0.553 | 0.012 | 0.539 | −0.189 | −0.289 | 0.160 | 0.020 | −0.617 |
| MCHR1-Tango | −0.853 | −0.099 | −0.157 | −0.373 | −0.561 | −0.988 | −0.951 | −1.223 |
| MCHR2-Tango | −0.201 | −0.158 | −0.185 | −0.604 | −0.735 | −0.705 | −0.605 | −0.609 |
| MLNR-Tango | −0.464 | −0.265 | 0.110 | −0.734 | −0.883 | −0.152 | −0.727 | −0.332 |
| MRGPRD-Tango | −0.481 | −0.156 | 0.120 | −0.539 | −0.644 | −0.887 | −1.018 | −0.578 |
| MRGPRE-Tango | −0.091 | −0.420 | 0.298 | −0.493 | −0.837 | −0.537 | −0.751 | −0.178 |
| MRGPRF-Tango | −0.595 | −0.456 | 0.340 | −0.614 | −0.937 | −0.435 | −0.505 | −0.320 |
| MRGPRG-Tango | −0.514 | −0.209 | 0.403 | −0.588 | −0.988 | −0.365 | −0.628 | −0.507 |
| MRGPRX1-Tango | −0.863 | −0.133 | 0.295 | −0.111 | −0.771 | −0.867 | −0.498 | −1.056 |
| MRGPRX2-Tango | −0.317 | −0.418 | −0.091 | −0.977 | −1.366 | −1.446 | −1.373 | −0.400 |
| MRGPRX3-Tango | −0.197 | −0.133 | −0.079 | −0.485 | −1.000 | −0.788 | −0.968 | −0.621 |
| MRGPRX4-Tango | −0.685 | −0.420 | 0.312 | −1.005 | −1.548 | −0.619 | −0.452 | −1.226 |
| MTNR1A-Tango | −0.301 | −0.476 | 0.374 | −0.762 | −1.168 | 0.000 | 0.140 | −0.119 |
| MTNR1B-Tango | −0.613 | −0.735 | 0.051 | −0.639 | −0.756 | −1.114 | −0.816 | 0.931 |
| NMBR-Tango | 0.444 | 0.063 | 0.273 | −0.480 | −0.805 | 0.109 | −0.100 | 0.479 |
| NMUR1-Tango | −0.626 | −0.520 | 0.174 | −0.087 | −0.315 | 0.317 | −0.341 | −0.785 |
| NMUR2-Tango | −0.966 | −0.642 | 0.533 | 0.000 | −0.409 | −0.170 | −0.082 | −1.118 |
| NPBW1-Tango | −0.298 | 0.260 | −0.366 | −0.640 | −1.887 | −0.350 | −0.115 | −0.554 |
| NPBW2-Tango | −0.716 | −0.608 | −0.976 | −1.473 | −1.619 | −0.739 | −0.776 | −0.975 |
| NPFF1-Tango | −0.031 | −0.350 | 0.160 | −0.675 | −1.663 | −0.345 | −0.467 | −0.261 |
| NPFF2-Tango | −0.153 | −0.439 | 0.363 | 0.227 | −0.520 | −0.028 | 0.023 | −0.326 |
| NPS-Tango | −0.672 | −0.015 | −0.360 | −0.133 | −0.853 | −0.422 | −0.211 | −0.722 |
| NPY1R-Tango | 0.000 | 0.061 | −0.223 | 0.074 | −0.706 | −0.428 | −0.193 | −0.348 |
| NPY2R-Tango | −0.367 | −0.255 | 0.169 | −0.358 | −1.109 | −0.289 | −0.346 | −0.196 |
| NPY4R-Tango | −0.774 | −0.149 | −0.316 | −0.585 | −0.954 | 0.000 | 0.000 | −0.798 |
| NPY5R-Tango | −0.126 | −0.861 | 0.358 | −0.509 | −1.069 | −0.763 | −1.169 | −0.325 |
| NTSR1-Tango | 0.036 | −0.971 | 0.312 | −0.915 | −1.748 | −0.408 | −0.488 | −0.298 |
| NTSR2-Tango | −0.221 | −0.693 | −0.110 | −0.119 | −1.082 | −0.841 | −0.743 | −0.413 |
| OPN3-Tango | 0.267 | 0.212 | 0.425 | −0.857 | −1.066 | −0.231 | −0.357 | −0.148 |
| OPN5-Tango | 0.207 | −1.096 | 0.469 | −0.813 | −1.785 | −1.037 | −0.828 | −0.543 |
| OPRD1-Tango | −1.027 | −0.959 | −0.352 | −0.628 | −0.582 | −0.754 | −0.637 | −1.013 |
| OPRK1-Tango | −0.625 | −0.186 | 0.604 | −0.182 | −0.501 | −0.049 | −0.054 | −0.275 |
| OPRL1-Tango | −0.612 | −0.128 | 0.241 | −0.501 | −0.857 | −0.336 | −0.411 | −0.303 |
| OPRM1-Tango | −0.417 | −0.334 | 0.055 | −0.291 | −0.599 | −0.520 | −0.054 | −0.257 |
| OXER1-Tango | −0.150 | −0.400 | 0.282 | −0.558 | −0.899 | −0.253 | −0.202 | −0.191 |
| OXGR1-Tango | −0.657 | −0.915 | 0.087 | −0.623 | −0.806 | −0.686 | −0.706 | −0.608 |
| OXTR-Tango | 0.011 | −0.729 | 0.307 | −1.000 | −1.363 | −0.160 | −0.256 | −0.033 |
| P2RY1-Tango | −0.594 | −1.031 | 0.220 | −1.536 | −1.571 | −1.299 | −1.160 | −0.998 |
| P2RY10-Tango | −0.465 | −0.742 | 0.275 | −0.887 | −0.787 | −0.687 | −0.543 | −0.381 |
| P2RY11-Tango | −0.258 | −0.995 | 0.254 | −1.043 | −1.213 | −0.425 | −0.410 | −0.335 |
| P2RY12-Tango | −0.394 | −1.488 | 0.160 | −1.541 | −2.094 | −0.786 | −0.717 | −0.339 |
| P2RY13-Tango | −0.299 | −0.535 | 0.492 | −0.286 | −0.799 | −0.356 | −0.336 | −0.661 |
| P2RY14-Tango | −0.544 | −0.491 | 0.281 | −1.144 | −1.302 | −0.467 | −0.533 | −0.554 |
| P2RY2-Tango | −0.668 | −0.910 | 0.202 | −1.215 | −1.272 | −0.946 | −0.943 | −0.739 |
| P2RY4-Tango | −0.972 | −0.813 | 0.152 | −1.634 | −1.512 | −0.853 | −0.805 | −1.263 |
| P2RY6-Tango | −0.667 | −0.812 | 0.282 | −1.118 | −1.480 | −0.761 | −0.786 | −0.956 |
| P2RY8-Tango | −0.981 | −0.888 | 0.105 | −0.555 | −1.028 | −0.491 | −0.414 | −1.094 |
| PK1-Tango | −0.341 | 0.047 | 0.142 | −0.608 | −0.893 | −0.814 | −0.795 | −0.330 |
| PK2-Tango | 0.000 | 0.044 | 0.234 | −0.126 | −0.281 | −0.485 | 0.071 | 0.284 |
| PRRP-Tango | −0.141 | −0.436 | 0.413 | −0.732 | −1.353 | −0.529 | −0.529 | −0.016 |
| PTAFR-Tango | −0.155 | −0.823 | 0.285 | −1.206 | −1.178 | −0.838 | −1.238 | −0.370 |
| PTGDR-Tango | −0.159 | −0.633 | 0.096 | −1.071 | −1.071 | −0.643 | −0.909 | −0.422 |
| PTGER1-Tango | −0.092 | 0.164 | 0.000 | −0.858 | −0.573 | −0.043 | −0.165 | 0.065 |
| PTGER2-Tango | −0.296 | −0.543 | 0.604 | −0.241 | −0.358 | 0.295 | 0.361 | −0.184 |
| PTGER3-Tango | 0.123 | −0.462 | 0.488 | −0.436 | −0.647 | −0.882 | −0.758 | −0.210 |
| PTGER4-Tango | 0.023 | 0.503 | 0.842 | 0.000 | −0.677 | 0.080 | 0.011 | 0.278 |
| PTGFR-Tango | −0.435 | −0.387 | 0.611 | −0.048 | −0.420 | −0.730 | −0.458 | −0.527 |
| PTGIR-Tango | −0.133 | −0.193 | −0.874 | −0.521 | −0.471 | −1.866 | −0.670 | −0.382 |
| PTH1R-Tango | −0.498 | −0.521 | 0.381 | −0.737 | −1.100 | −0.230 | −0.042 | −0.306 |
| PTH2R-Tango | −0.641 | −0.890 | 0.254 | −0.627 | −0.932 | −0.390 | −0.665 | −0.363 |
| QRFP-Tango | −0.479 | −0.381 | 0.393 | −0.677 | −0.487 | −0.384 | −0.188 | −0.402 |
| RXFP1-Tango | −0.434 | −0.336 | −0.156 | −0.664 | −0.874 | −0.956 | −1.135 | −0.458 |
| RXFP2-Tango | −0.844 | −0.782 | 0.370 | −0.691 | −1.194 | −0.770 | −0.679 | −0.834 |
| RXFP3-Tango | −0.784 | −0.372 | −0.151 | −0.190 | −0.335 | −0.141 | −0.786 | −0.792 |
| RXFP4-Tango | 0.054 | −0.840 | 0.216 | 0.000 | 0.102 | −0.156 | −0.156 | −0.057 |
| S1PR1-Tango | 0.604 | −0.659 | 1.282 | 0.619 | 0.369 | −0.107 | −0.605 | 0.505 |
| S1PR2-Tango | 0.687 | −0.106 | 0.503 | 0.181 | 0.144 | −0.729 | −1.087 | 0.076 |
| S1PR3-Tango | 0.232 | 0.106 | 0.657 | 0.465 | 0.364 | −0.200 | −0.234 | −0.258 |
| S1PR4-Tango | 0.383 | −0.726 | 0.392 | −0.425 | −0.111 | −1.035 | −0.822 | −0.164 |
| S1PR5-Tango | 0.326 | −0.553 | −0.126 | −0.607 | −0.220 | −0.627 | −0.521 | −0.216 |
| SCTR-Tango | 0.670 | 0.063 | −0.009 | −0.156 | −0.076 | −0.335 | −0.302 | 0.117 |
| SSTR1-Tango | −0.930 | 0.176 | 0.675 | 0.145 | 0.345 | 0.244 | 0.563 | −0.832 |
| SSTR2-Tango | 0.531 | 0.037 | 0.519 | 0.705 | −0.170 | −0.661 | −0.415 | 0.655 |
| SSTR3-Tango | 0.328 | 0.625 | 0.592 | 0.913 | 0.122 | −0.415 | −0.819 | 0.585 |
| SSTR4-Tango | −0.685 | 0.000 | −0.186 | −0.102 | 0.095 | −0.665 | −0.665 | 0.077 |
| SSTR5-Tango | 0.260 | 1.793 | 0.511 | 0.564 | 0.347 | 0.563 | 0.048 | 0.451 |
| SUCNR1-Tango | 2.335 | 3.592 | 3.198 | 2.538 | 2.624 | 2.500 | 2.186 | 2.453 |
| TA1-Tango | −0.423 | −0.020 | 0.259 | −0.365 | −0.651 | −0.429 | −0.509 | −0.150 |
| TAAR2-Tango | −0.141 | −0.466 | 0.143 | 0.016 | 0.060 | −0.673 | −0.739 | −0.029 |
| TAAR5-Tango | −0.517 | −0.539 | −0.381 | −0.477 | −0.442 | −0.230 | −0.541 | −0.566 |
| TAAR6-Tango | −0.683 | −0.380 | −0.559 | −0.662 | −0.730 | −0.342 | −0.604 | −0.901 |
| TAAR8-Tango | −0.336 | −0.718 | −0.071 | −0.642 | −0.503 | −0.054 | −0.085 | −0.757 |
| TAAR9-Tango | −0.714 | −0.616 | 0.266 | −0.282 | −0.952 | −0.199 | −0.217 | −0.794 |
| TACR1-Tango | −0.516 | −0.521 | −0.092 | −0.596 | −1.064 | −0.416 | −0.421 | −0.477 |
| TACR2-Tango | −0.245 | −0.340 | 0.696 | −0.049 | −0.641 | −0.544 | −0.571 | −0.010 |
| TACR3-Tango | −0.353 | −0.304 | 0.319 | −0.325 | −0.938 | −1.104 | −0.875 | −0.100 |
| TBXA2R-Tango | 0.040 | −0.587 | 0.176 | −0.531 | −0.766 | 0.154 | −0.672 | 0.120 |
| TSHR-Tango | −0.810 | −0.412 | −0.306 | −0.906 | −1.087 | −0.414 | −0.414 | −0.674 |
| UTS2R-Tango | −0.144 | 0.086 | −0.288 | 0.369 | −0.982 | −0.260 | 0.050 | −0.334 |
| VIPR1-Tango | −0.863 | −0.594 | 0.232 | −0.550 | −1.093 | −1.188 | −0.809 | −0.767 |
| VIPR2-Tango | −0.333 | 0.990 | 1.354 | −0.143 | −0.503 | −1.527 | −0.645 | −1.000 |

| PRESTO-Tango | Bacteroides | Bacteroides | Bacteroides | Bacteroides | Bacteroides | Bacteroides | Bacteroides | Parabacteroides |
|---|---|---|---|---|---|---|---|---|
| with GMM | spp-2# | spp-3# | spp-4# | spp-5# | UC-1# | UC-2# | UC-3# | distasonis-1# |
| ADCYAP1R1- | −0.583 | −0.890 | −0.495 | −0.229 | 0.167 | −0.190 | −1.456 | 0.551 |
| Tango | | | | | | | | |
| ADORA1-Tango | −0.995 | −0.951 | −0.768 | −0.832 | −0.621 | −0.594 | −0.921 | −0.598 |
| ADORA2A-Tango | −0.225 | −0.025 | −0.533 | −0.442 | −0.387 | −1.154 | −0.052 | −0.025 |
| ADORA2B-Tango | −0.365 | −0.066 | −0.303 | −0.367 | −0.281 | −0.751 | −0.215 | −0.318 |
| ADORA3-Tango | −0.153 | 0.650 | −0.387 | −0.051 | 0.266 | 0.003 | −0.499 | 0.019 |
| ADRA1A-Tango | −0.120 | 0.161 | −0.681 | 0.000 | −0.055 | −0.355 | −0.619 | −0.142 |
| ADRA1B-Tango | −0.363 | −0.254 | −0.590 | −0.368 | 0.032 | −0.732 | −0.501 | −0.584 |
| ADRA1D-Tango | −0.549 | −0.345 | −0.521 | −0.009 | −0.616 | 0.018 | −0.616 | 0.356 |
| ADRA2A-Tango | −0.430 | −0.015 | −0.197 | −1.052 | 0.185 | −1.309 | −0.429 | −0.357 |
| ADRA2B-Tango | −0.859 | −1.026 | −1.277 | −1.103 | −0.834 | −1.144 | −0.995 | −0.383 |
| ADRA2C-Tango | −0.678 | −0.077 | −0.970 | −0.208 | −0.405 | −1.036 | 0.037 | −0.246 |
| ADRB1-Tango | −0.224 | −0.188 | 0.354 | −0.642 | 0.659 | −0.705 | −0.385 | 0.032 |
| ADRB2-Tango | −0.453 | −0.974 | −0.363 | 0.199 | −0.170 | 0.333 | −0.423 | 0.000 |
| ADRB3-Tango | 0.000 | −1.000 | −0.165 | −0.405 | −0.213 | −0.405 | −0.234 | −0.397 |
| AGTR1-Tango | −0.244 | 0.147 | −0.016 | 0.079 | 0.213 | −0.415 | 0.226 | 0.409 |
| AGTR2-Tango | 0.039 | 0.264 | −0.234 | 0.180 | 0.344 | 0.101 | −0.567 | −0.132 |
| APJ-Tango | −0.635 | −0.141 | −0.862 | −0.215 | −0.829 | 0.000 | −0.628 | −0.339 |
| AVPR1A-Tango | −0.642 | 0.034 | −0.459 | 0.018 | −0.290 | −0.667 | −0.023 | −0.355 |
| AVPR1B-Tango | −0.496 | −0.150 | −0.489 | −0.349 | −0.112 | −0.816 | −0.111 | 0.040 |
| AVPR2-Tango | −0.665 | −0.360 | −0.814 | −1.037 | −0.651 | −0.762 | −0.501 | −1.019 |
| BB3-Tango | −0.759 | −0.770 | −0.730 | −1.178 | −0.432 | −0.949 | −0.792 | −0.617 |
| BDKBR1-Tango | 0.099 | 0.050 | −0.588 | −0.564 | −0.259 | −0.875 | −0.180 | −0.341 |
| BDKBR2-Tango | −0.166 | 0.555 | −0.089 | 0.332 | 0.252 | 0.084 | −0.283 | −0.081 |
| C3AR1-Tango | −0.577 | −0.497 | −0.593 | −0.612 | −0.140 | −0.809 | −0.760 | −0.711 |
| C5A-Tango | 0.150 | 0.123 | −0.312 | 0.254 | 0.066 | −0.034 | −0.060 | −0.278 |
| CALCRb-Tango | −0.163 | −0.062 | −0.149 | −0.445 | −0.256 | −0.680 | −0.376 | −0.046 |
| CALCRL-Tango | −0.087 | 0.027 | −0.454 | 0.127 | −0.491 | −0.569 | −0.056 | −0.206 |
| CASR-Tango | −0.481 | −0.593 | −0.378 | −0.400 | −0.265 | −0.467 | −0.082 | −1.060 |
| CCKAR-Tango | −0.157 | −0.228 | −0.204 | −0.119 | 0.217 | −0.301 | 0.093 | 0.107 |
| CCKBR-Tango | −0.276 | −0.336 | −0.726 | −0.266 | −0.410 | −0.351 | −0.344 | −0.936 |
| CCR10-Tango | −0.589 | −0.511 | −0.212 | −0.531 | −0.396 | −0.622 | −0.513 | −0.806 |
| CCR2-Tango | 0.248 | 0.106 | −0.671 | 0.184 | −0.575 | −0.252 | −0.789 | −1.661 |
| CCR3-Tango | 0.089 | 0.110 | −0.197 | 0.313 | 0.080 | −0.021 | −0.859 | −0.720 |
| CCR4-Tango | −0.362 | −0.343 | −0.383 | −0.124 | −0.078 | −0.101 | −0.650 | −0.143 |
| CCR5-Tango | 0.073 | 0.219 | −0.088 | −0.012 | 0.095 | −0.349 | −0.180 | 0.280 |
| CCR6-Tango | 0.498 | 0.123 | 0.304 | 0.574 | 0.633 | 0.206 | −0.201 | 0.376 |
| CCR7-Tango | −0.378 | −0.470 | −0.054 | −0.125 | 0.120 | −0.771 | −0.497 | −0.330 |
| CCR8-Tango | −0.265 | −0.212 | −0.300 | −0.284 | −0.187 | −0.284 | −0.708 | 0.181 |
| CCRL2-Tango | 0.017 | −0.318 | −0.341 | −0.108 | 0.064 | −0.585 | 0.613 | −0.077 |
| CD97-Tango | −0.649 | −0.329 | −0.606 | −0.780 | −0.899 | −0.468 | −0.788 | −0.937 |
| CHRM1-Tango | −0.627 | −0.307 | −0.384 | −1.367 | −1.117 | −1.050 | −0.027 | 0.325 |
| CHRM2-Tango | −1.099 | −1.089 | −0.815 | −1.094 | −0.589 | −1.318 | −0.492 | −0.513 |
| CHRM3-Tango | −1.005 | −1.198 | −0.938 | −0.643 | −0.778 | −1.264 | −0.630 | 0.277 |
| CHRM4-Tango | −0.453 | −0.217 | 0.115 | −0.031 | 0.377 | −0.490 | −0.130 | 0.284 |
| CHRM5-Tango | −0.571 | −0.529 | 0.133 | −0.377 | 0.260 | −0.255 | −0.660 | 0.576 |
| CMKLR1-Tango | −0.296 | −0.154 | −0.074 | −0.227 | −0.017 | −0.517 | −0.039 | −0.296 |
| CNR1-Tango | −0.641 | −0.770 | −0.450 | −0.546 | −0.645 | −0.469 | −0.036 | −0.458 |
| CNR2-Tango | −0.192 | −0.412 | −0.908 | −0.537 | −0.495 | −0.500 | −0.109 | 0.202 |
| CRHR1-Tango | 0.108 | 0.037 | −0.458 | 0.117 | −0.506 | −0.127 | −0.703 | 0.255 |
| CRHR2-Tango | −0.052 | 0.165 | −0.404 | −0.819 | 0.172 | −1.396 | −0.537 | −0.405 |
| CX3C1-Tango | 0.150 | 0.087 | −0.750 | −0.721 | −0.390 | −0.674 | −0.471 | −0.034 |
| CXCR1-Tango | −0.114 | 0.023 | −0.018 | −0.441 | 0.537 | −0.400 | 0.067 | 0.573 |
| CXCR2-Tango | −0.500 | −0.628 | −0.138 | −0.110 | 0.373 | 0.291 | −0.956 | 0.700 |
| CXCR3-Tango | −0.410 | −0.289 | −0.215 | 0.134 | −0.240 | −0.100 | 0.155 | 0.326 |
| CXCR4-Tango | −0.610 | −0.353 | 0.066 | 0.011 | 0.445 | −0.258 | −0.320 | 0.549 |
| CXCR5-Tango | −0.309 | −0.143 | −0.431 | −0.157 | 0.036 | −0.103 | −0.146 | 0.292 |
| CXCR6-Tango | −0.372 | −0.147 | 0.022 | −0.819 | −0.642 | −0.920 | 0.196 | 1.000 |
| CXCR7-Tango | −1.169 | −0.784 | −1.397 | −0.796 | −1.099 | −0.389 | −1.048 | −0.280 |
| CYSLTR1-Tango | −0.759 | −0.229 | −0.629 | −0.171 | 0.003 | −0.030 | −0.598 | 0.490 |
| CYSLTR2-Tango | −0.315 | −0.493 | −0.114 | 0.079 | 0.023 | −0.698 | 0.159 | 0.592 |
| DRD1-Tango | −0.813 | −0.997 | −0.371 | −0.361 | −0.229 | −0.384 | −0.925 | 0.809 |
| DRD2-Tango | −0.448 | −0.187 | −0.080 | −1.275 | −0.671 | −2.112 | −1.538 | 1.093 |
| DRD3-Tango | −1.091 | −0.948 | −0.087 | −2.246 | 0.160 | −2.709 | 0.136 | 1.381 |
| DRD4-Tango | −0.690 | −0.690 | 1.062 | −1.597 | 0.000 | −3.121 | −1.322 | 1.140 |
| DRD5-Tango | −0.358 | −0.232 | −0.661 | −0.646 | −0.587 | −0.770 | −0.584 | −0.079 |
| EDNRA-Tango | −0.369 | −0.414 | −0.436 | −0.350 | −0.243 | −0.303 | −0.541 | 0.441 |
| EDNRB-Tango | −0.457 | −0.323 | −0.765 | −0.445 | −0.604 | −0.445 | −0.208 | −0.376 |
| ELTD1-Tango | −0.922 | −0.692 | −0.870 | −0.632 | −0.725 | −0.423 | −1.002 | −0.800 |
| F2R-Tango | −0.230 | −0.057 | −0.319 | −0.131 | −0.069 | −0.306 | −0.202 | −0.105 |
| F2RL1-Tango | −0.160 | −0.058 | 0.482 | −0.161 | −0.154 | −0.332 | −0.199 | −0.057 |
| F2RL2-Tango | −0.722 | −0.353 | −0.133 | −0.585 | 0.166 | −0.773 | −0.252 | −0.281 |
| F2RL3-Tango | −0.775 | −0.280 | −0.361 | −0.141 | −0.168 | −0.621 | −0.486 | −0.061 |
| FFA1-Tango | −0.727 | −0.576 | −0.088 | −0.150 | 0.053 | 0.012 | −0.258 | −0.339 |
| FFA2-Tango | −0.963 | −0.578 | −0.367 | −0.407 | −0.417 | −0.432 | −0.121 | −0.319 |
| FFA3-Tango | −0.575 | −0.346 | 0.061 | −0.120 | 0.000 | −0.535 | −0.396 | 0.367 |
| FPR1-Tango | −0.966 | −0.455 | −1.343 | −1.430 | −1.362 | −1.301 | −1.657 | −0.546 |
| FPR2-Tango | −0.299 | −0.212 | −0.599 | −0.640 | −0.468 | −1.067 | 0.077 | 0.488 |
| FPR3-Tango | −0.670 | −0.355 | −0.493 | −0.403 | −0.245 | −0.428 | −0.365 | 0.010 |
| FSHR-Tango | −0.645 | −0.596 | −0.548 | −0.470 | −0.482 | −0.736 | −0.534 | −0.352 |
| GABBR1-Tango | 0.000 | −0.139 | −0.384 | −0.790 | −0.384 | −0.727 | −0.178 | 0.038 |
| GAL1-Tango | −0.740 | −0.740 | −1.430 | −0.748 | −2.062 | −0.595 | −0.893 | −1.222 |
| GAL2-Tango | −0.514 | −0.147 | −0.878 | −0.707 | −1.216 | −1.209 | −0.084 | 0.885 |
| GAU-Tango | −0.672 | −0.126 | −1.383 | −1.209 | −2.101 | −1.472 | −0.491 | −0.209 |
| GCGR-Tango | −0.849 | −0.651 | −0.826 | −0.533 | −0.465 | −0.678 | −0.702 | −0.573 |
| GHRHR-Tango | −0.248 | −0.802 | −0.357 | −0.399 | −0.435 | −1.177 | −0.786 | −0.408 |
| GHSR-Tango | −0.705 | −0.389 | −0.177 | −0.508 | −0.152 | −0.499 | −1.030 | −2.232 |
| GIPR-Tango | −0.769 | −0.359 | −0.020 | −0.607 | −0.405 | −0.607 | −0.732 | −0.491 |
| GLP1R-Tango | −0.462 | −0.029 | −1.175 | −0.991 | −0.844 | −1.564 | −0.996 | −0.024 |
| GLP2R-Tango | −0.120 | −0.364 | −0.768 | 0.184 | −0.768 | −0.112 | −0.372 | 0.089 |
| GNRHR-Tango | −0.253 | −0.183 | −0.122 | −0.745 | −0.480 | −0.872 | 0.089 | −1.000 |
| GPBA-Tango | −0.379 | −0.379 | −1.272 | −0.399 | −0.735 | −0.077 | 0.347 | −1.063 |
| GPER-Tango | −0.076 | −0.034 | −0.164 | −0.245 | −1.189 | −0.569 | −0.282 | 0.075 |
| GPR1-Tango | −0.508 | −0.567 | −0.353 | −1.025 | −0.527 | −1.170 | −1.166 | −0.787 |
| GPR101-Tango | −0.411 | −0.387 | −0.295 | −0.603 | −0.025 | −0.672 | −0.174 | −0.536 |
| GPR110-Tango | −0.345 | −0.493 | −0.978 | −0.807 | −0.709 | −0.996 | −0.369 | −0.043 |
| GPR111-Tango | −0.057 | −0.509 | −0.627 | −0.968 | −0.579 | −1.422 | −0.468 | 0.083 |
| GPR113-Tango | −0.521 | −0.847 | −0.666 | −0.021 | −0.700 | −0.521 | −0.781 | −0.511 |
| GPR114-Tango | −0.260 | −0.400 | −0.778 | −0.391 | −0.182 | −0.493 | −0.438 | −0.262 |
| GPR115-Tango | −0.917 | −1.032 | −0.785 | −0.367 | −0.318 | −0.472 | −0.725 | −0.287 |
| GPR116-Tango | −0.424 | −0.616 | −0.886 | −0.485 | −0.499 | −0.351 | −0.594 | −0.613 |
| GPR119-Tango | −0.339 | −0.278 | −0.351 | −0.401 | −0.509 | −0.821 | −0.213 | −0.384 |
| GPR12-Tango | −0.326 | −0.300 | −0.135 | −0.106 | 0.255 | −0.305 | −0.718 | −0.119 |
| GPR120-Tango | 0.272 | 0.016 | −0.242 | −0.401 | −0.065 | −0.524 | −0.326 | −0.105 |
| GPR123-Tango | −0.291 | −0.019 | −0.192 | −0.448 | 0.221 | −0.696 | −0.486 | 0.131 |
| GPR124-Tango | −0.627 | −0.818 | −0.926 | −0.494 | −0.620 | −0.229 | −0.043 | 0.223 |
| GPR125-Tango | −0.914 | −0.799 | −0.588 | −0.305 | 0.160 | −0.139 | −0.362 | 0.551 |
| GPR126-Tango | −0.743 | −0.681 | −1.017 | −0.749 | −0.762 | −0.727 | −0.366 | −0.200 |
| GPR132-Tango | −0.810 | −0.824 | −1.028 | −0.545 | −0.611 | −0.995 | −0.268 | −0.014 |
| GPR133-Tango | −0.866 | −0.876 | −0.724 | −0.406 | −0.449 | −0.379 | −0.277 | −0.237 |
| GPR135-Tango | −0.478 | −0.562 | −0.604 | −0.325 | −0.199 | −0.376 | −0.339 | 0.147 |
| GPR141-Tango | −0.514 | −0.339 | −0.267 | −0.308 | −0.127 | −0.130 | −0.422 | 0.335 |
| GPR142-Tango | −0.831 | −0.820 | −0.738 | −0.011 | −0.501 | −0.061 | −0.243 | −0.331 |
| GPR143-Tango | −0.672 | −0.585 | −0.511 | −0.395 | −0.143 | −1.230 | −0.283 | −0.087 |
| GPR144-Tango | −0.858 | −0.545 | −1.222 | −0.929 | −1.033 | −1.000 | −0.560 | −0.918 |
| GPR146-Tango | −0.565 | −0.395 | 0.170 | −0.685 | 0.104 | −0.525 | 0.000 | −0.472 |
| GPR148-Tango | 0.181 | 0.775 | −1.122 | 0.074 | −0.402 | −0.407 | 0.828 | 0.778 |
| GPR149-Tango | −0.522 | −0.785 | −0.250 | −0.939 | −0.310 | −1.064 | −1.006 | −0.658 |
| GPR15-Tango | −0.543 | −0.477 | −0.554 | −0.229 | 0.077 | −0.500 | −0.483 | −0.462 |
| GPR150-Tango | −0.091 | −0.335 | −0.350 | −0.068 | 0.160 | 0.127 | 0.040 | −0.686 |
| GPR151-Tango | −0.820 | −0.526 | −0.330 | −0.475 | −0.284 | −0.503 | 0.095 | −0.435 |
| GPR152-Tango | −0.480 | −0.545 | −0.756 | 0.005 | −0.886 | −0.208 | 0.510 | −0.165 |
| GPR153-Tango | −0.694 | −0.595 | −0.240 | −0.516 | 0.052 | −0.446 | 0.461 | 0.250 |
| GPR156-Tango | −0.578 | −0.372 | −0.793 | −0.652 | −0.800 | −0.430 | −0.101 | −0.319 |
| GPR157-Tango | −0.598 | −0.402 | 0.242 | −0.239 | −0.269 | −0.603 | 0.263 | −0.102 |
| GPR158-Tango | −0.114 | −0.258 | −0.994 | −0.471 | −0.978 | −0.174 | 0.196 | −0.113 |
| GPR160-Tango | −0.695 | −0.460 | −0.238 | −0.046 | −0.209 | −0.281 | 0.674 | 0.009 |
| GPR161-Tango | −0.308 | −0.413 | 0.108 | −0.385 | 0.130 | −0.283 | 0.409 | 0.135 |
| GPR162-Tango | −0.623 | −0.678 | −0.258 | −0.421 | −0.326 | −0.688 | −0.064 | −0.196 |
| GPR17-Tango | −0.910 | −1.103 | 0.093 | 0.044 | 0.151 | 0.197 | −0.187 | 0.117 |
| GPR171-Tango | −0.555 | −0.854 | −0.117 | −0.009 | −0.117 | −0.078 | −0.040 | −0.137 |
| GPR173-Tango | −0.705 | −0.352 | −0.081 | −0.045 | −0.293 | 0.153 | −0.129 | −0.361 |
| GPR174-Tango | −0.306 | −0.068 | −0.060 | 0.308 | −0.157 | −0.212 | −0.180 | 0.474 |
| GPR18-Tango | −0.369 | −0.120 | −0.145 | −0.338 | −0.177 | −0.331 | −0.094 | −0.165 |
| GPR182-Tango | −0.916 | −0.949 | 0.141 | −0.387 | 0.066 | −0.259 | −0.321 | −0.157 |
| GPR183-Tango | −0.404 | −0.369 | −0.376 | −0.060 | −0.284 | −0.186 | −0.149 | 0.106 |
| GPR19-Tango | −0.814 | −0.576 | −0.087 | −0.086 | 0.069 | −0.207 | −0.219 | −0.020 |
| GPR20-Tango | −0.098 | −0.241 | −0.112 | −0.585 | 0.511 | −0.481 | 0.489 | 0.170 |
| GPR21-Tango | 0.000 | −0.146 | −0.563 | −0.437 | −0.719 | −0.905 | −0.080 | 0.030 |
| GPR22-Tango | 0.044 | 0.194 | −0.013 | −0.147 | −0.169 | −0.428 | −0.115 | 0.349 |
| GPR25-Tango | 0.132 | −0.019 | −0.178 | −0.226 | −0.537 | −0.589 | −0.211 | 0.265 |
| GPR26-Tango | −0.535 | −0.307 | 0.436 | −0.280 | 0.700 | −0.280 | −0.195 | −0.429 |
| GPR27-Tango | −0.063 | −0.063 | 0.308 | −0.302 | −0.016 | −0.511 | 0.104 | 0.969 |
| GPR3-Tango | −0.707 | −1.002 | −0.081 | −0.125 | −0.150 | 0.328 | −0.084 | 0.657 |
| GPR31-Tango | −0.737 | −0.713 | 0.142 | −0.336 | −0.072 | −0.453 | −0.314 | 0.447 |
| GPR32-Tango | −0.204 | −0.183 | −0.103 | −0.646 | −0.255 | −0.426 | −0.527 | −0.491 |
| GPR34-Tango | −0.431 | −0.261 | −1.090 | −1.374 | −0.628 | −1.040 | −0.704 | −0.264 |
| GPR35-Tango | −0.663 | −0.588 | −0.572 | −0.461 | −0.167 | −0.633 | −0.525 | −0.374 |
| GPR37-Tango | −0.289 | −0.012 | −0.040 | −0.924 | 0.362 | −0.899 | −0.518 | 0.260 |
| GPR37L1-Tango | −0.327 | −0.481 | −0.382 | −0.212 | 0.304 | −0.397 | −0.562 | −0.292 |
| GPR39-Tango | −0.564 | −0.344 | −0.612 | −0.390 | −0.604 | −0.414 | −0.346 | −0.446 |
| GPR4-Tango | −0.449 | −0.572 | −0.299 | −0.758 | 0.264 | −0.711 | −0.669 | 0.471 |
| GPR44-Tango | −0.456 | −0.545 | −0.503 | −0.520 | −0.176 | −0.964 | −0.610 | −0.014 |
| GPR45-Tango | −0.229 | −0.387 | −0.252 | −0.403 | −0.293 | −0.525 | −1.054 | −0.824 |
| GPR50-Tango | 0.089 | 0.451 | 0.115 | 0.158 | 0.677 | −0.206 | −0.101 | 0.896 |
| GPR52-Tango | −0.089 | 0.081 | 0.289 | −0.074 | 0.424 | −0.713 | −0.530 | 0.635 |
| GPR55-Tango | −0.266 | −0.016 | −0.046 | −0.970 | 0.311 | −0.782 | −0.440 | −0.090 |
| GPR56-Tango | 0.839 | 1.109 | 1.301 | −0.628 | 1.224 | −0.403 | −0.475 | 0.217 |
| GPR6-Tango | −0.047 | −0.047 | 0.294 | −0.321 | 0.503 | −0.261 | −0.348 | 0.240 |
| GPR61-Tango | −0.408 | 0.315 | 0.088 | −0.160 | 0.604 | −0.640 | −0.289 | 0.439 |
| GPR62-Tango | 0.048 | −0.191 | −0.200 | −0.184 | 0.000 | −0.292 | −0.025 | 0.058 |
| GPR63-Tango | −0.612 | −0.954 | −0.790 | −0.453 | −0.358 | −0.715 | −0.452 | −0.212 |
| GPR64-Tango | −0.544 | −0.641 | −0.629 | −0.850 | −0.538 | −0.682 | −0.775 | −0.098 |
| GPR65-Tango | −0.148 | −0.344 | −0.446 | −0.494 | −0.026 | −0.713 | −0.307 | 0.322 |
| GPR68-Tango | −0.401 | −0.441 | −0.606 | −0.764 | −0.005 | −1.123 | −0.839 | 0.212 |
| GPR75-Tango | 0.242 | 0.526 | 0.016 | −0.144 | 0.243 | −0.733 | −0.464 | 0.480 |
| GPR77-Tango | −0.162 | 0.039 | −0.312 | −0.754 | −0.249 | −1.176 | −0.980 | −1.180 |
| GPR78-Tango | 0.074 | 0.347 | 0.448 | 0.069 | 0.635 | −0.481 | −0.025 | 0.279 |
| GPR82-Tango | −0.534 | 0.028 | −0.063 | −0.255 | 0.015 | −0.749 | −0.755 | 0.092 |
| GPR83-Tango | −0.017 | 0.084 | −0.382 | −0.290 | −0.095 | −0.695 | −0.372 | 0.114 |
| GPR84-Tango | −0.300 | −0.231 | −0.421 | −0.369 | 0.225 | −0.730 | −0.573 | 0.305 |
| GPR85-Tango | 0.154 | 0.089 | −0.610 | −0.172 | −0.298 | −0.464 | −0.265 | 1.017 |
| GPR87-Tango | −0.188 | 0.077 | −0.140 | −0.203 | 0.395 | −0.312 | −0.368 | 0.432 |
| GPR88-Tango | −0.007 | 0.104 | −0.300 | −0.490 | 0.089 | −0.781 | −0.038 | 0.411 |
| GPR97-Tango | −0.097 | 0.212 | −0.295 | −0.445 | −0.165 | −0.787 | −0.578 | −0.270 |
| GPRC5A-Tango | −0.174 | 0.153 | −0.088 | −0.339 | 0.145 | −0.404 | −0.581 | −0.395 |
| GPRC5B-Tango | −0.271 | 0.272 | −0.415 | −0.293 | −0.029 | −0.467 | −0.452 | 0.284 |
| GPRC5C-Tango | 0.410 | 0.241 | −0.358 | −0.023 | 0.048 | −0.258 | −0.378 | 0.473 |
| GPRC5D-Tango | 0.017 | 0.021 | −0.807 | −0.617 | −0.460 | −0.709 | −0.429 | −0.160 |
| GPRC6A-Tango | −0.002 | −0.090 | −1.281 | −0.491 | −0.790 | −0.628 | −0.648 | 0.205 |
| GRM1-Tango | 0.032 | −0.309 | −1.198 | −0.153 | −0.747 | −0.178 | −0.789 | 0.214 |
| GRM2-Tango | −0.350 | −0.170 | −0.856 | −0.760 | −0.688 | −0.880 | −0.709 | −0.305 |
| GRM4-Tango | 0.114 | 0.119 | −0.399 | −0.698 | −0.165 | −1.096 | −0.379 | 0.367 |
| GRM5-Tango | −0.403 | −0.209 | −1.166 | −0.984 | −0.378 | −1.233 | −0.309 | 0.236 |
| GRM6-Tango | −0.210 | −0.330 | −0.745 | −0.850 | −0.184 | −0.877 | −0.773 | 0.256 |
| GRM7-Tango | −0.190 | −0.161 | −1.645 | −0.848 | −1.463 | −1.094 | −1.253 | −0.318 |
| GRM8-Tango | −0.539 | −0.350 | −0.829 | −0.806 | −0.361 | −0.922 | −0.531 | −0.294 |
| GRPR-Tango | 0.335 | 0.086 | 0.014 | 0.074 | −0.199 | −0.615 | −0.389 | 0.000 |
| HCA1-Tango | 0.163 | −0.124 | 0.144 | 0.457 | −0.230 | −0.257 | −0.481 | −0.077 |
| HCA2-Tango | 0.241 | 0.272 | −0.217 | −0.356 | 0.028 | −0.428 | −0.433 | 0.246 |
| HCA3-Tango | −0.328 | −0.440 | −0.382 | 0.471 | −0.755 | 0.077 | −0.193 | 0.065 |
| HCTR1-Tango | −0.521 | −0.111 | −0.078 | 0.122 | −0.623 | 0.000 | −0.261 | 0.087 |
| HCTR2-Tango | −0.549 | 0.106 | −0.686 | −1.234 | −0.255 | −0.112 | 0.060 | −1.280 |
| HRH1-Tango | −0.774 | −0.605 | −0.098 | −0.473 | 0.329 | −0.212 | −0.203 | 0.174 |
| HRH2-Tango | −0.569 | −0.253 | −0.068 | −0.069 | −0.022 | −0.377 | 0.132 | 0.511 |
| HRH3-Tango | −0.269 | 0.516 | −0.442 | −0.515 | −0.456 | 0.454 | −0.068 | 0.634 |
| HRH4-Tango | −0.075 | 0.228 | −0.231 | −0.310 | 0.160 | −0.037 | −0.144 | 0.716 |
| HTR1A-Tango | 0.080 | −0.007 | 0.143 | −0.215 | 0.587 | −0.374 | −0.078 | 0.736 |
| HTR1B-Tango | −0.079 | −0.273 | −0.111 | −0.636 | 0.446 | −0.618 | −0.173 | 0.686 |
| HTR1D-Tango | −0.210 | −0.476 | −0.182 | −0.374 | 0.168 | −0.312 | 0.000 | 0.404 |
| HTR1E-Tango | −0.509 | −0.293 | 0.189 | −0.223 | 0.789 | −0.297 | −0.097 | 0.347 |
| HTR1F-Tango | −0.237 | −0.224 | 0.182 | −0.434 | 0.451 | −0.454 | −0.699 | 0.838 |
| HTR2A-Tango | −0.066 | −0.277 | −0.066 | −0.747 | 0.148 | −0.471 | −0.534 | 0.276 |
| HTR2B-Tango | −0.210 | −0.491 | −0.585 | −0.911 | −0.317 | −0.915 | −0.598 | 0.607 |
| HTR2C-Tango | −0.032 | −0.320 | −0.101 | −0.595 | −0.259 | −0.827 | −0.360 | −0.187 |
| HTR4-Tango | −0.264 | −0.342 | −0.208 | −0.197 | 0.243 | −0.795 | −0.592 | 0.029 |
| HTR5-Tango | −0.443 | 0.035 | 0.373 | 0.209 | 0.504 | −0.241 | −0.566 | 0.768 |
| HTR6-Tango | 0.431 | 0.274 | −0.259 | −0.455 | −0.136 | −0.573 | 0.082 | 0.200 |
| HTR7-Tango | 0.275 | 0.743 | 0.041 | −0.785 | 0.397 | −0.605 | −0.281 | 0.487 |
| KISSPEPTIN-R- | −0.267 | 0.477 | 0.204 | 0.127 | 0.000 | −0.531 | 0.073 | 0.000 |
| Tango | | | | | | | | |
| LHCGR-Tango | −0.231 | −0.055 | −0.716 | −0.089 | −0.422 | −0.142 | −0.541 | −0.300 |
| LPAR1-Tango | −0.290 | 0.364 | −0.352 | −0.430 | 0.108 | −0.529 | −0.448 | −0.155 |
| LPAR2-Tango | −0.384 | −0.045 | −0.540 | −0.343 | −0.421 | −0.814 | −0.138 | −0.582 |
| LPAR4-Tango | −0.275 | −0.161 | −0.154 | −0.670 | −0.012 | −1.036 | −0.529 | −0.477 |
| LPAR5-Tango | −0.287 | −0.334 | −0.439 | −0.642 | −0.017 | −0.702 | −0.542 | −0.934 |
| LPAR6-Tango | −0.146 | 0.408 | −0.132 | −0.095 | 0.544 | −0.251 | −0.394 | 0.543 |
| LTB4R-Tango | −0.175 | 0.076 | 0.165 | −0.153 | 0.537 | −0.117 | −0.178 | −0.027 |
| LTB4R2B-Tango | −0.142 | 0.082 | −0.407 | 0.115 | 0.188 | −0.373 | −0.450 | 0.129 |
| MAS1-Tango | −0.074 | −0.162 | −0.327 | −0.338 | 0.006 | −0.294 | −0.582 | 0.256 |
| MAS1L-Tango | −0.213 | −0.081 | −0.303 | −0.644 | 0.005 | −0.721 | −0.497 | −0.459 |
| MC1R-Tango | 0.096 | −0.079 | −0.204 | −0.354 | 0.278 | −0.605 | −0.127 | −0.446 |
| MC2R-Tango | 0.107 | 0.171 | −0.055 | −0.482 | −0.055 | −0.317 | −0.495 | 0.426 |
| MC3R-Tango | 0.239 | 0.995 | 0.364 | 0.132 | 0.267 | −1.216 | −0.720 | 0.560 |
| MC4R-Tango | 0.166 | 0.953 | −0.100 | −0.122 | −0.429 | −0.032 | −0.467 | 0.395 |
| MC5R-Tango | 0.531 | 1.081 | 0.344 | 0.339 | 0.314 | −0.585 | −0.441 | 0.344 |
| MCHR1-Tango | −1.287 | −0.819 | −0.232 | −0.177 | −0.417 | −0.368 | −0.996 | −0.599 |
| MCHR2-Tango | −0.430 | −0.170 | −0.018 | −0.835 | −0.129 | −0.568 | −0.312 | −0.296 |
| MLNR-Tango | −0.134 | −0.391 | −0.363 | −0.381 | 0.116 | −1.064 | 0.280 | 0.244 |
| MRGPRD-Tango | −0.696 | −0.723 | −0.011 | −0.357 | 0.357 | −0.285 | −0.438 | −0.157 |
| MRGPRE-Tango | −0.209 | −0.143 | −0.604 | −0.484 | −0.329 | −0.588 | −0.238 | −0.039 |
| MRGPRF-Tango | −0.113 | 0.008 | −0.055 | −0.520 | 0.461 | −0.608 | −0.779 | 0.038 |
| MRGPRG-Tango | −0.622 | −0.815 | −0.448 | −0.793 | 0.199 | −0.636 | −0.831 | 0.027 |
| MRGPRX1-Tango | −0.766 | −0.905 | −0.334 | −0.067 | −0.029 | −0.236 | −0.807 | 0.013 |
| MRGPRX2-Tango | −0.220 | −0.554 | −0.185 | −1.030 | −0.160 | −1.112 | −0.832 | −0.262 |
| MRGPRX3-Tango | −0.327 | −0.420 | −0.417 | −0.485 | −0.487 | −1.340 | −0.482 | −0.141 |
| MRGPRX4-Tango | −0.882 | −0.361 | −1.011 | −1.332 | −0.372 | −1.673 | −1.183 | −0.815 |
| MTNR1A-Tango | −0.126 | 0.126 | −0.050 | −0.784 | −0.172 | −0.700 | −0.956 | −0.109 |
| MTNR1B-Tango | −0.058 | −0.352 | 0.117 | −0.334 | 0.032 | −0.443 | −0.762 | −0.273 |
| NMBR-Tango | 0.161 | 0.316 | 0.137 | 0.009 | −0.182 | −0.891 | −0.594 | 0.119 |
| NMUR1-Tango | −0.564 | −0.234 | −0.545 | 0.328 | −0.545 | −0.315 | −0.762 | −0.406 |
| NMUR2-Tango | 0.357 | 0.290 | −0.569 | −0.290 | −0.642 | −0.097 | −0.833 | −0.363 |
| NPBW1-Tango | −0.463 | −0.108 | 0.523 | −0.955 | 0.480 | −1.406 | 0.368 | 0.631 |
| NPBW2-Tango | −0.672 | −0.195 | −0.560 | −1.247 | −0.163 | −1.266 | −0.550 | −0.323 |
| NPFF1-Tango | 0.220 | −0.052 | −0.128 | −0.675 | −0.267 | −1.212 | −0.331 | −0.458 |
| NPFF2-Tango | 0.139 | 0.575 | −0.388 | 0.171 | −0.173 | −0.237 | −0.329 | −0.776 |
| NPS-Tango | 0.005 | 0.163 | −0.865 | 0.280 | −0.030 | −0.519 | −0.489 | −0.317 |
| NPY1R-Tango | 0.005 | 0.421 | −0.143 | −0.050 | 0.259 | −0.876 | −0.403 | −1.168 |
| NPY2R-Tango | 0.014 | 0.416 | −0.815 | −0.822 | 0.104 | −1.578 | −0.336 | −0.020 |
| NPY4R-Tango | −0.540 | −0.126 | −0.805 | −0.628 | −0.504 | −1.350 | −0.444 | −0.148 |
| NPY5R-Tango | −0.213 | −0.168 | −0.710 | −0.468 | −0.344 | −1.293 | −0.452 | −0.249 |
| NTSR1-Tango | −0.365 | 0.108 | −1.005 | −1.108 | −0.544 | −1.637 | −0.715 | −0.506 |
| NTSR2-Tango | 0.125 | 0.125 | −0.561 | −0.119 | −0.229 | −0.720 | −0.544 | −0.386 |
| OPN3-Tango | 0.000 | −0.077 | 0.537 | −0.311 | 0.165 | −0.896 | −0.155 | 0.027 |
| OPN5-Tango | −0.643 | −0.748 | −0.600 | −0.863 | −0.056 | −0.966 | −1.033 | −0.608 |
| OPRD1-Tango | −0.325 | −0.551 | −0.749 | −0.440 | −0.595 | −0.715 | −0.586 | −0.018 |
| OPRK1-Tango | 0.146 | 0.401 | −0.113 | −0.307 | 0.269 | −0.277 | 0.160 | 0.294 |
| OPRL1-Tango | −0.206 | −0.159 | −0.202 | −0.831 | −0.092 | −1.143 | −0.205 | 0.585 |
| OPRM1-Tango | 0.242 | 0.463 | −0.484 | −0.493 | −0.553 | −0.084 | −0.503 | −1.406 |
| OXER1-Tango | −0.380 | 0.203 | −0.322 | −0.835 | 0.066 | −0.683 | −0.804 | −0.288 |
| OXGR1-Tango | −0.587 | −0.201 | −0.480 | −0.204 | −0.343 | −0.548 | −0.759 | −0.943 |
| OXTR-Tango | −0.036 | 0.348 | −0.352 | −0.509 | −0.481 | −0.848 | −0.550 | −0.085 |
| P2RY1-Tango | −0.410 | −0.333 | −0.844 | −0.971 | −0.793 | −0.756 | −0.177 | −0.004 |
| P2RY10-Tango | −0.017 | 0.020 | −0.805 | −0.945 | −0.832 | −0.552 | −0.216 | −0.200 |
| P2RY11-Tango | −0.564 | −0.286 | −1.324 | −0.585 | −0.859 | −0.852 | −0.449 | −0.284 |
| P2RY12-Tango | −0.246 | 0.121 | −0.292 | −1.455 | −0.620 | −1.290 | −1.377 | −0.199 |
| P2RY13-Tango | −0.576 | −0.744 | −0.389 | −0.261 | −0.169 | −0.310 | −0.336 | −0.237 |
| P2RY14-Tango | −1.017 | −0.653 | −0.505 | −0.996 | −0.167 | −0.852 | −0.238 | −0.273 |
| P2RY2-Tango | −0.910 | −0.944 | −0.784 | −1.056 | −0.617 | −1.099 | −0.517 | −0.305 |
| P2RY4-Tango | −1.134 | −1.261 | −0.666 | −1.008 | −0.481 | −0.957 | −0.707 | 0.011 |
| P2RY6-Tango | −0.868 | −1.128 | −0.831 | −0.952 | −0.415 | −1.037 | −0.210 | 0.288 |
| P2RY8-Tango | −1.023 | −1.245 | −0.775 | −0.372 | −0.500 | −0.849 | −0.700 | −0.026 |
| PK1-Tango | −0.627 | −0.720 | −0.110 | −0.893 | −0.300 | −0.475 | −0.220 | 0.036 |
| PK2-Tango | −0.377 | −0.467 | −1.317 | −0.227 | −0.585 | −0.435 | −0.013 | 0.355 |
| PRRP-Tango | −0.465 | −0.472 | −0.337 | −1.183 | −0.450 | −0.832 | −0.106 | 0.050 |
| PTAFR-Tango | −0.685 | −0.468 | −0.687 | −1.056 | −0.446 | −0.850 | −0.411 | 0.579 |
| PTGDR-Tango | −0.426 | −0.534 | −0.443 | −0.780 | −0.317 | −0.496 | −0.408 | −0.185 |
| PTGER1-Tango | −0.023 | 0.188 | 0.292 | −0.600 | 0.037 | −0.366 | −0.071 | −0.420 |
| PTGER2-Tango | 0.183 | −0.339 | −0.463 | 0.165 | −0.399 | 0.000 | −0.146 | 0.397 |
| PTGER3-Tango | −0.589 | −0.672 | −0.419 | −0.267 | −0.397 | −0.225 | −0.612 | 0.134 |
| PTGER4-Tango | 0.250 | 0.667 | 0.339 | −0.128 | 0.517 | −0.019 | −0.240 | 0.696 |
| PTGFR-Tango | −0.316 | −0.036 | −0.200 | 0.077 | 0.098 | −0.923 | −0.512 | 0.260 |
| PTGIR-Tango | −0.627 | −1.093 | −0.437 | −0.073 | −0.553 | −0.423 | 0.045 | 1.148 |
| PTH1R-Tango | −0.091 | −0.286 | −0.931 | −0.814 | −0.301 | −0.535 | −0.436 | 0.477 |
| PTH2R-Tango | −1.150 | −1.345 | −1.214 | −1.006 | −0.738 | −0.443 | −0.921 | 0.606 |
| QRFP-Tango | −0.084 | −0.350 | −0.780 | −0.627 | −1.070 | −0.647 | −0.410 | 0.243 |
| RXFP1-Tango | −0.764 | −1.139 | −0.606 | 0.085 | −0.408 | −0.430 | −0.871 | −0.419 |
| RXFP2-Tango | −1.158 | −1.117 | −1.071 | −0.587 | −0.824 | −0.857 | −0.813 | 0.123 |
| RXFP3-Tango | −0.323 | −0.167 | −0.750 | −0.277 | −1.229 | −0.556 | −1.168 | −0.483 |
| RXFP4-Tango | −0.790 | −1.025 | −0.888 | −0.054 | −1.122 | −0.054 | −0.907 | −0.062 |
| S1PR1-Tango | −0.580 | −0.410 | −0.428 | 0.539 | 0.420 | 0.564 | −0.439 | −0.458 |
| S1PR2-Tango | −0.741 | −0.391 | −0.362 | 0.144 | 0.208 | −0.144 | −0.378 | −0.183 |
| S1PR3-Tango | 0.215 | −0.505 | −0.240 | 0.465 | 0.100 | 0.000 | −0.448 | 0.230 |
| S1PR4-Tango | −0.402 | 0.366 | −0.234 | −0.482 | 0.285 | 0.572 | −0.869 | −0.856 |
| S1PR5-Tango | −0.125 | 0.132 | −0.523 | 0.000 | 0.000 | 0.336 | −0.394 | 0.721 |
| SCTR-Tango | 0.077 | 0.263 | 0.284 | −0.200 | 0.210 | 0.060 | −0.009 | 0.766 |
| SSTR1-Tango | 0.000 | 0.189 | 0.364 | −0.260 | 0.603 | −0.052 | −0.476 | −0.538 |
| SSTR2-Tango | 0.547 | 0.493 | 0.091 | 0.078 | 0.883 | −0.329 | −0.593 | 0.179 |
| SSTR3-Tango | 0.272 | 0.077 | 0.564 | 0.497 | 0.411 | −0.415 | −0.316 | 1.148 |
| SSTR4-Tango | 0.164 | 0.000 | 0.729 | 0.255 | 0.940 | −0.050 | −0.378 | 0.913 |
| SSTR5-Tango | 0.733 | 0.489 | 2.043 | 0.807 | 2.316 | 0.198 | 0.651 | 0.684 |
| SUCNR1-Tango | 2.558 | 2.703 | 3.885 | 3.097 | 4.249 | 3.176 | 3.289 | 2.569 |
| TA1-Tango | −0.340 | 0.101 | 0.085 | 0.118 | 0.427 | −0.595 | −0.275 | 1.193 |
| TAAR2-Tango | 0.065 | −0.076 | −0.345 | 0.307 | 0.050 | 0.132 | −0.420 | 0.143 |
| TAAR5-Tango | −0.552 | −0.539 | −0.562 | −0.131 | 0.009 | −0.230 | −0.284 | 0.079 |
| TAAR6-Tango | −0.875 | −0.770 | −0.484 | −0.607 | 0.373 | −0.816 | −0.759 | −0.347 |
| TAAR8-Tango | −0.608 | −0.750 | −0.987 | 0.090 | −0.335 | −0.413 | −0.556 | −0.300 |
| TAAR9-Tango | −0.518 | −0.716 | −0.632 | −0.315 | −0.428 | −0.422 | −0.554 | −0.581 |
| TACR1-Tango | −0.693 | −0.624 | −0.496 | −0.621 | −0.149 | −0.933 | −0.640 | 0.079 |
| TACR2-Tango | −0.503 | −0.604 | −0.233 | −0.132 | −0.063 | −0.444 | −0.368 | 0.050 |
| TACR3-Tango | −0.374 | −0.652 | −0.308 | −0.348 | −0.049 | −0.730 | −0.697 | −0.126 |
| TBXA2R-Tango | −0.409 | −0.693 | −0.423 | −0.503 | −0.280 | −0.788 | −0.428 | −0.392 |
| TSHR-Tango | −0.842 | −1.096 | −0.529 | −0.491 | −0.020 | −0.816 | −0.539 | −0.192 |
| UTS2R-Tango | −0.086 | −0.199 | −0.050 | 0.251 | 0.260 | −0.094 | 0.029 | −1.097 |
| VIPR1-Tango | −0.256 | −0.083 | −0.148 | −0.673 | −0.089 | −0.759 | −0.924 | −0.993 |
| VIPR2-Tango | −0.023 | 0.725 | 1.685 | −0.387 | 1.578 | −0.260 | 0.240 | 1.255 |

| PRESTO-Tango | Parabacteroides | Fusobacterium | Fusobacterium | Collinsella | Collinsella | Collinsella | Collinsella | Collinsella |
|---|---|---|---|---|---|---|---|---|
| with GMM | distasonis-2# | spp-1# | spp-2# | aerofaciens-1# | aerofaciens-2# | aerofaciens-3# | stercoris-1# | stercoris-2# |
| ADCYAP1R1- | −0.731 | −0.891 | −1.163 | −0.043 | −0.262 | 0.024 | 0.105 | −0.159 |
| Tango | | | | | | | | |
| ADORA1-Tango | −0.779 | −1.830 | −1.785 | −1.003 | −0.847 | −0.492 | −0.515 | −0.310 |
| ADORA2A-Tango | −0.083 | −0.711 | −0.260 | −0.227 | −0.461 | 0.234 | −0.440 | −0.425 |
| ADORA2B-Tango | −0.437 | −0.443 | −0.706 | −0.269 | −0.432 | 0.011 | −0.210 | −0.433 |
| ADORA3-Tango | −0.260 | −0.615 | −0.703 | 0.240 | 0.035 | 0.443 | −0.177 | 0.110 |
| ADRA1A-Tango | −0.111 | −0.532 | −0.786 | −0.134 | −0.474 | −0.071 | −0.133 | −0.328 |
| ADRA1B-Tango | −0.002 | −0.734 | −0.881 | 0.154 | 0.181 | 0.252 | −0.380 | 0.115 |
| ADRA1D-Tango | −0.427 | −0.921 | −0.699 | −0.089 | −0.363 | −0.204 | −0.470 | 0.180 |
| ADRA2A-Tango | −0.086 | −0.346 | −0.803 | −0.175 | −0.367 | 0.612 | −0.412 | −0.473 |
| ADRA2B-Tango | −1.200 | −1.807 | −0.993 | −0.903 | −0.720 | −0.144 | −0.750 | −0.157 |
| ADRA2C-Tango | 0.626 | −0.462 | −0.267 | −0.882 | −0.197 | 0.908 | −0.158 | −0.030 |
| ADRB1-Tango | −0.886 | −0.463 | −1.303 | 0.032 | −0.160 | 0.727 | 0.760 | 0.288 |
| ADRB2-Tango | −0.774 | −0.993 | −1.068 | 0.265 | 0.126 | 0.645 | 0.124 | −0.086 |
| ADRB3-Tango | 0.352 | −0.434 | −0.148 | 0.350 | 0.507 | −0.127 | 0.156 | −0.104 |
| AGTR1-Tango | 0.419 | −0.968 | −0.934 | 0.011 | −0.016 | 0.088 | 0.073 | −0.154 |
| AGTR2-Tango | −0.069 | −1.347 | −1.307 | −0.217 | −0.233 | 0.049 | 0.325 | 0.190 |
| APJ-Tango | −0.455 | −1.765 | −1.070 | −0.624 | −0.716 | 0.437 | −0.143 | 0.365 |
| AVPR1A-Tango | −0.390 | −1.118 | −0.955 | 0.401 | 0.348 | 0.459 | −0.322 | 0.097 |
| AVPR1B-Tango | 0.025 | −1.213 | −1.214 | 0.216 | 0.051 | 0.233 | −0.082 | 0.031 |
| AVPR2-Tango | −0.337 | −1.248 | −1.524 | −0.396 | −0.366 | 0.254 | −0.050 | 0.234 |
| BB3-Tango | −0.689 | −1.328 | −1.706 | −0.370 | −0.447 | −0.246 | 0.129 | −0.065 |
| BDKBR1-Tango | 0.117 | −0.473 | −0.764 | −0.313 | −0.228 | 0.426 | 0.241 | 0.307 |
| BDKBR2-Tango | 0.150 | −1.147 | −1.200 | 0.193 | 0.287 | 0.434 | 0.399 | 0.266 |
| C3AR1-Tango | −0.665 | −0.992 | −1.255 | −0.101 | −0.032 | 0.060 | −0.192 | 0.000 |
| C5A-Tango | 0.023 | −0.887 | −0.719 | 0.483 | 0.407 | 0.435 | 0.198 | 0.047 |
| CALCRb-Tango | −0.242 | −0.056 | −0.548 | 0.287 | 0.024 | 0.200 | −0.021 | −0.010 |
| CALCRL-Tango | 0.057 | −0.147 | −0.244 | −0.144 | −0.040 | −0.112 | −0.205 | −0.373 |
| CASR-Tango | −0.046 | −1.160 | −1.011 | 0.181 | 0.385 | 0.276 | 0.081 | 0.330 |
| CCKAR-Tango | 0.059 | −1.068 | −0.878 | 0.323 | 0.274 | 0.129 | 0.058 | 0.305 |
| CCKBR-Tango | −0.344 | −0.902 | −0.725 | −0.382 | −0.218 | 0.231 | −0.014 | 0.234 |
| CCR10-Tango | −0.363 | −0.705 | −0.938 | 0.048 | 0.138 | 0.234 | −0.130 | 0.183 |
| CCR2-Tango | −0.555 | −0.903 | −0.666 | −0.336 | −0.147 | 0.562 | 0.067 | 0.413 |
| CCR3-Tango | −0.753 | −1.669 | −1.862 | −0.426 | −0.526 | −0.268 | −0.202 | −0.091 |
| CCR4-Tango | −0.589 | −1.184 | −0.915 | 0.742 | 0.556 | 0.592 | −0.074 | 0.310 |
| CCR5-Tango | 0.000 | −0.948 | −0.889 | 0.507 | 0.751 | 0.252 | 0.075 | 0.159 |
| CCR6-Tango | 0.000 | −0.962 | −1.307 | 0.758 | 0.657 | 0.531 | 0.057 | 0.435 |
| CCR7-Tango | −0.808 | −1.187 | −0.766 | 0.537 | 0.333 | 0.120 | 0.026 | 0.035 |
| CCR8-Tango | −0.818 | −1.076 | −0.585 | −0.187 | −0.320 | 0.623 | −0.204 | 0.313 |
| CCRL2-Tango | −0.718 | −0.932 | −0.415 | 0.285 | 0.354 | 0.291 | −0.468 | −0.468 |
| CD97-Tango | −0.053 | −1.285 | −1.437 | −1.234 | −0.671 | 0.184 | −0.140 | −0.054 |
| CHRM1-Tango | 0.422 | −0.198 | −0.260 | −0.184 | −0.754 | 0.252 | 0.000 | −0.337 |
| CHRM2-Tango | 0.471 | −1.398 | −1.469 | −0.650 | −0.403 | 0.259 | 0.196 | 0.262 |
| CHRM3-Tango | −0.041 | −0.415 | −0.807 | −0.497 | −0.628 | 0.343 | 0.021 | 0.121 |
| CHRM4-Tango | 0.171 | −0.274 | −0.442 | 0.151 | 0.327 | 0.208 | 0.034 | 0.067 |
| CHRM5-Tango | 0.092 | −1.193 | −1.101 | 0.220 | 0.355 | 0.112 | 0.458 | 0.196 |
| CMKLR1-Tango | 0.004 | −0.677 | −0.778 | 0.216 | 0.255 | 0.302 | 0.050 | 0.134 |
| CNR1-Tango | 0.064 | −1.211 | −0.742 | 0.112 | 0.148 | 0.392 | −0.023 | 0.406 |
| CNR2-Tango | 0.000 | −0.923 | −1.101 | −0.319 | −0.300 | 0.126 | −0.158 | 0.196 |
| CRHR1-Tango | −0.292 | −1.079 | −0.722 | −0.146 | −0.411 | 0.260 | 0.068 | −0.234 |
| CRHR2-Tango | −0.112 | −0.826 | −0.845 | 0.039 | −0.218 | 0.367 | −0.069 | 0.217 |
| CX3C1-Tango | −0.444 | −0.451 | −0.520 | −0.230 | −0.451 | 0.552 | 0.220 | −0.118 |
| CXCR1-Tango | −0.033 | −1.210 | 0.094 | 0.479 | 0.418 | 0.705 | 0.109 | 0.024 |
| CXCR2-Tango | 0.028 | −0.481 | −0.343 | −0.365 | −0.419 | −0.072 | 0.661 | 0.202 |
| CXCR3-Tango | −0.016 | −0.994 | −0.554 | −0.348 | −0.590 | 0.375 | 0.214 | 0.178 |
| CXCR4-Tango | 0.266 | −0.949 | −1.401 | 0.003 | −0.135 | −0.100 | 0.420 | 0.082 |
| CXCR5-Tango | 0.163 | −0.198 | −0.604 | −0.121 | −0.282 | 0.117 | 0.197 | 0.099 |
| CXCR6-Tango | 0.028 | −0.705 | −0.769 | 0.524 | 0.340 | 0.595 | 0.239 | 0.227 |
| CXCR7-Tango | −0.412 | −1.854 | −1.919 | −0.746 | −0.709 | 0.122 | 0.277 | −0.028 |
| CYSLTR1-Tango | −0.006 | −1.508 | −0.679 | −0.231 | −0.288 | −0.049 | 0.236 | 0.054 |
| CYSLTR2-Tango | 0.088 | −0.815 | −0.140 | 0.245 | 0.225 | 0.489 | 0.299 | 0.108 |
| DRD1-Tango | −0.562 | −1.904 | −1.199 | 0.231 | 0.271 | 0.866 | −0.383 | 0.008 |
| DRD2-Tango | −0.882 | −1.117 | −2.062 | −0.223 | 0.000 | 1.027 | −0.140 | 0.060 |
| DRD3-Tango | 0.190 | −0.918 | −0.737 | 0.477 | 0.415 | 2.232 | −0.068 | −0.386 |
| DRD4-Tango | −0.070 | −0.745 | −1.322 | 0.867 | 1.122 | 2.078 | 0.290 | −0.108 |
| DRD5-Tango | −0.666 | −1.067 | −0.388 | −0.241 | −0.347 | −0.148 | −0.315 | −0.357 |
| EDNRA-Tango | −0.208 | −1.255 | −0.688 | 0.158 | 0.219 | 0.325 | 0.186 | 0.509 |
| EDNRB-Tango | 0.119 | −0.366 | −0.263 | −0.245 | −0.245 | 0.148 | 0.229 | −0.206 |
| ELTD1-Tango | −0.658 | −1.939 | −1.141 | −0.709 | −0.555 | −0.379 | −0.055 | 0.013 |
| F2R-Tango | 0.054 | −1.532 | −0.859 | 0.389 | 0.575 | 0.319 | −0.134 | 0.211 |
| F2RL1-Tango | 0.276 | −1.596 | −0.784 | 0.356 | 0.621 | 0.202 | 0.065 | 0.366 |
| F2RL2-Tango | 0.036 | −2.049 | −1.239 | 0.392 | 0.440 | 0.228 | −0.024 | 0.278 |
| F2RL3-Tango | −0.393 | −1.311 | −1.161 | 0.139 | 0.286 | 0.280 | 0.077 | 0.476 |
| FFA1-Tango | 0.125 | −1.446 | −0.904 | −0.168 | −0.211 | 0.086 | 0.397 | 0.225 |
| FFA2-Tango | 0.166 | −0.890 | −0.615 | −0.167 | −0.083 | 0.249 | −0.002 | 0.045 |
| FFA3-Tango | 0.242 | −1.556 | −0.809 | 0.796 | 0.735 | 0.537 | 0.076 | 0.336 |
| FPR1-Tango | 0.508 | −1.664 | −0.882 | 1.101 | 1.004 | 1.038 | 0.788 | −0.064 |
| FPR2-Tango | 0.558 | −0.879 | −0.079 | 0.628 | 0.494 | 0.941 | 0.201 | 0.292 |
| FPR3-Tango | 0.170 | −1.603 | −1.043 | −0.289 | −0.249 | 0.014 | 0.161 | 0.255 |
| FSHR-Tango | −0.199 | −1.615 | −0.941 | −0.196 | −0.189 | −0.039 | −0.150 | −0.066 |
| GABBR1-Tango | 0.124 | −0.364 | −0.140 | −0.091 | −0.125 | 0.455 | −0.038 | −0.276 |
| GAL1-Tango | −1.074 | −1.293 | −0.611 | −1.174 | −1.080 | 0.252 | 0.112 | −0.373 |
| GAL2-Tango | −0.258 | −1.737 | −1.627 | −0.094 | −0.216 | 0.698 | −0.335 | 0.026 |
| GAL3-Tango | −0.391 | −0.827 | −0.680 | −0.197 | −0.235 | 0.889 | 0.133 | 0.435 |
| GCGR-Tango | −0.618 | −0.909 | −0.493 | 0.072 | −1.076 | 0.314 | −0.655 | −0.206 |
| GHRHR-Tango | −0.442 | −0.780 | −0.942 | 0.100 | −0.180 | 0.494 | 0.365 | 0.177 |
| GHSR-Tango | −1.166 | −1.640 | −1.785 | −0.101 | 0.030 | 0.114 | −0.308 | 0.085 |
| GIPR-Tango | −0.834 | −1.149 | −1.132 | 0.415 | 0.430 | −0.247 | −0.850 | −0.284 |
| GLP1R-Tango | −1.171 | −1.572 | −1.557 | −0.014 | 0.189 | 0.416 | −0.596 | 0.464 |
| GLP2R-Tango | −0.148 | −1.191 | −1.326 | −0.179 | −0.187 | 0.047 | −0.289 | 0.088 |
| GNRHR-Tango | −0.379 | −0.626 | −0.285 | 0.043 | 0.229 | 0.038 | −0.039 | 0.264 |
| GPBA-Tango | −0.081 | −0.453 | −0.690 | −0.401 | −0.607 | 0.432 | −0.142 | 0.367 |
| GPER-Tango | −0.660 | −0.758 | −0.731 | −0.393 | −0.092 | 0.317 | 0.064 | 0.037 |
| GPR1-Tango | −0.702 | −1.218 | −0.853 | −0.126 | −0.072 | 0.004 | 0.033 | 0.369 |
| GPR101-Tango | 0.246 | −0.970 | −0.448 | −0.439 | −0.186 | 0.125 | 0.348 | 0.261 |
| GPR110-Tango | −0.274 | −1.376 | −1.679 | −0.395 | −0.169 | 0.154 | −0.444 | −0.079 |
| GPR111-Tango | −0.077 | −0.781 | −0.696 | −0.550 | −0.135 | 0.372 | 0.102 | −0.087 |
| GPR113-Tango | −0.781 | −1.037 | −0.481 | 0.000 | 0.165 | 0.145 | −0.383 | −0.177 |
| GPR114-Tango | −0.266 | −0.147 | 0.066 | −0.010 | −0.102 | −0.099 | −0.126 | −0.306 |
| GPR115-Tango | 0.159 | −0.930 | −0.870 | −0.226 | −0.294 | −0.191 | −0.271 | −0.136 |
| GPR116-Tango | −0.421 | −0.656 | −1.018 | −0.640 | −0.315 | −0.256 | −0.566 | −0.586 |
| GPR119-Tango | 0.040 | −0.347 | −0.538 | −0.060 | −0.203 | 0.187 | −0.097 | −0.377 |
| GPR12-Tango | −0.562 | −1.160 | −1.314 | −0.373 | −0.166 | 0.361 | 0.268 | 0.473 |
| GPR120-Tango | 0.078 | −0.072 | −0.575 | 0.268 | 0.288 | 0.040 | 0.167 | −0.109 |
| GPR123-Tango | −0.072 | −1.045 | −0.817 | 0.078 | 0.310 | 0.227 | −0.291 | 0.050 |
| GPR124-Tango | 0.131 | −0.649 | −1.454 | −0.170 | −0.271 | −0.106 | −0.426 | −0.371 |
| GPR125-Tango | 0.046 | −0.921 | −1.519 | −0.171 | −0.065 | −0.484 | −0.349 | −0.482 |
| GPR126-Tango | −0.002 | −0.878 | −1.169 | −0.318 | −0.372 | −0.143 | −0.395 | −0.210 |
| GPR132-Tango | −0.344 | −1.343 | −1.170 | −0.380 | −0.321 | 0.031 | −0.647 | 0.094 |
| GPR133-Tango | 0.332 | −1.308 | −1.755 | −0.367 | −0.304 | −0.337 | −0.430 | −0.353 |
| GPR135-Tango | −0.021 | −0.866 | −0.836 | −0.168 | −0.044 | 0.104 | −0.038 | −0.336 |
| GPR141-Tango | −0.269 | −0.397 | −1.515 | −0.204 | −0.596 | 0.652 | 0.526 | 0.449 |
| GPR142-Tango | −0.382 | −0.913 | −1.745 | −0.174 | 0.067 | 0.512 | −0.604 | 0.308 |
| GPR143-Tango | −0.530 | −1.717 | −0.850 | 0.444 | 0.569 | 0.729 | −0.168 | 0.722 |
| GPR144-Tango | −0.298 | −1.443 | −1.142 | −0.596 | −0.501 | 0.192 | 0.000 | 0.087 |
| GPR146-Tango | −0.369 | −0.982 | −0.554 | 0.632 | 0.350 | 0.260 | 0.435 | 0.208 |
| GPR148-Tango | 0.700 | −0.503 | −1.904 | −0.122 | −1.122 | 0.000 | −0.087 | 0.160 |
| GPR149-Tango | −0.051 | −1.452 | −1.374 | 0.000 | 0.541 | 0.262 | 0.193 | −0.180 |
| GPR15-Tango | −0.700 | −1.449 | −0.705 | 0.206 | 0.458 | 0.377 | −0.394 | 0.829 |
| GPR150-Tango | 0.140 | −0.251 | −0.420 | 0.415 | 0.282 | 0.752 | 0.251 | 0.156 |
| GPR151-Tango | 0.421 | −1.532 | −0.781 | −0.128 | 0.118 | 0.755 | 0.404 | 0.816 |
| GPR152-Tango | −0.005 | −0.603 | −0.994 | −0.004 | −0.204 | 0.139 | 0.149 | 0.109 |
| GPR153-Tango | 0.133 | −1.374 | −1.290 | 0.293 | 0.250 | 0.042 | 0.275 | 0.113 |
| GPR156-Tango | 0.104 | −0.228 | −1.223 | −0.789 | −0.779 | −0.126 | −0.113 | −0.486 |
| GPR157-Tango | 0.202 | −0.287 | −0.913 | 0.200 | 0.129 | 0.588 | 0.255 | 0.034 |
| GPR158-Tango | −0.093 | −0.624 | −0.844 | −0.418 | −0.023 | 0.115 | −0.198 | −0.364 |
| GPR160-Tango | 0.075 | −0.532 | −0.557 | 0.527 | 0.513 | 0.506 | 0.236 | 0.358 |
| GPR161-Tango | 0.134 | −0.978 | −1.103 | 0.337 | 0.398 | 0.148 | 0.402 | 0.142 |
| GPR162-Tango | −0.281 | −0.575 | −0.704 | 0.006 | 0.014 | −0.089 | −0.030 | −0.219 |
| GPR17-Tango | 0.407 | −0.929 | −0.885 | 0.154 | 0.089 | 0.064 | 0.588 | 0.307 |
| GPR171-Tango | 0.247 | −0.200 | −0.436 | 0.063 | 0.214 | 0.521 | 0.312 | 0.392 |
| GPR173-Tango | 0.078 | −0.476 | −0.651 | 0.326 | 0.084 | 0.346 | 0.166 | 0.027 |
| GPR174-Tango | −0.006 | −0.738 | −0.705 | −0.032 | 0.198 | 0.049 | 0.275 | 0.082 |
| GPR18-Tango | −0.143 | −1.021 | −0.672 | 0.125 | −0.023 | 0.181 | 0.489 | 0.330 |
| GPR182-Tango | 0.002 | −1.222 | −1.007 | 0.076 | 0.190 | −0.334 | 0.149 | 0.060 |
| GPR183-Tango | −0.184 | −0.791 | −0.646 | 0.069 | 0.020 | 0.394 | 0.311 | 0.130 |
| GPR19-Tango | −0.221 | −1.096 | −1.087 | 0.440 | 0.448 | 0.142 | 0.074 | 0.097 |
| GPR20-Tango | 0.285 | −1.122 | −0.312 | 0.350 | 0.433 | 0.222 | 0.186 | 0.082 |
| GPR21-Tango | 0.036 | −0.670 | −0.712 | −0.267 | −0.128 | 0.324 | 0.076 | −0.185 |
| GPR22-Tango | 0.145 | −0.953 | −1.280 | 0.294 | 0.358 | 0.260 | 0.157 | 0.173 |
| GPR25-Tango | −0.077 | −0.805 | −0.473 | −0.129 | −0.335 | 0.700 | 0.666 | 0.551 |
| GPR26-Tango | −0.419 | −1.225 | −1.310 | 0.564 | 0.322 | 0.131 | 0.111 | 0.657 |
| GPR27-Tango | 0.192 | 0.143 | −0.365 | −0.160 | 0.070 | 0.408 | 0.677 | 0.512 |
| GPR3-Tango | 0.121 | −1.521 | −1.314 | −0.271 | −0.372 | 0.142 | 0.972 | 0.428 |
| GPR31-Tango | −0.187 | −1.444 | −0.993 | −0.228 | −0.038 | 0.151 | 0.190 | 0.319 |
| GPR32-Tango | −0.471 | −0.508 | −0.628 | −0.160 | −0.691 | 0.057 | −0.578 | −0.110 |
| GPR34-Tango | −0.246 | −0.903 | −0.798 | −0.489 | −0.735 | 0.134 | 0.085 | 0.245 |
| GPR35-Tango | −0.291 | −1.580 | −1.554 | −0.366 | −0.228 | 0.284 | 0.374 | 0.218 |
| GPR37-Tango | 0.067 | −1.419 | −1.506 | −0.129 | −0.200 | 0.265 | 0.013 | 0.598 |
| GPR37L1-Tango | −0.163 | −0.629 | −1.228 | 0.038 | 0.162 | 0.015 | 0.209 | 0.246 |
| GPR39-Tango | −0.375 | −0.766 | −0.744 | −0.483 | −0.328 | 0.237 | 0.694 | 0.407 |
| GPR4-Tango | −0.176 | −1.198 | −1.181 | 0.153 | 0.095 | 0.353 | 0.359 | 0.487 |
| GPR44-Tango | −0.383 | −0.489 | −0.899 | −0.277 | −0.270 | 0.093 | 0.168 | −0.069 |
| GPR45-Tango | −1.020 | −1.170 | −0.463 | −0.146 | 0.057 | −0.131 | −0.216 | −0.216 |
| GPR50-Tango | 0.129 | −0.619 | −0.435 | 0.717 | 0.927 | 0.466 | 0.231 | 0.193 |
| GPR52-Tango | 0.119 | −0.856 | −0.856 | −0.062 | 0.227 | 0.457 | 0.665 | 0.651 |
| GPR55-Tango | −0.152 | −1.218 | −1.280 | 0.023 | 0.204 | 0.299 | 0.194 | 0.597 |
| GPR56-Tango | 0.377 | −1.540 | −2.197 | −0.118 | −0.020 | −0.165 | 0.111 | 0.521 |
| GPR6-Tango | 0.145 | −1.101 | −1.019 | 0.023 | 0.076 | 0.234 | 0.312 | 0.315 |
| GPR61-Tango | −0.022 | −1.750 | −1.151 | 0.429 | 0.609 | 0.472 | 0.464 | 0.637 |
| GPR62-Tango | 0.324 | −0.275 | −0.402 | −0.692 | 0.158 | −0.245 | −0.159 | −0.091 |
| GPR63-Tango | 0.072 | −0.693 | −1.011 | −0.328 | −0.334 | −0.216 | 0.042 | −0.338 |
| GPR64-Tango | −0.072 | −0.892 | −0.839 | −0.154 | −0.447 | −0.375 | −0.030 | −0.288 |
| GPR65-Tango | −0.121 | −0.881 | −0.488 | 0.026 | 0.029 | −0.248 | −0.222 | −0.156 |
| GPR68-Tango | −0.509 | −1.082 | −0.891 | −0.148 | −0.115 | −0.407 | −0.656 | −0.367 |
| GPR75-Tango | −0.342 | −1.034 | −0.347 | 0.140 | 0.231 | −0.095 | −0.225 | −0.048 |
| GPR77-Tango | −0.946 | −1.011 | −1.106 | −0.216 | −0.372 | 0.047 | −0.387 | 0.096 |
| GPR78-Tango | −0.124 | −1.050 | −0.575 | 0.395 | 0.590 | 0.204 | 0.028 | 0.103 |
| GPR82-Tango | −0.729 | −2.014 | −2.025 | −0.024 | −0.076 | −0.061 | −0.084 | −0.015 |
| GPR83-Tango | −0.380 | −0.930 | −0.972 | 0.177 | 0.098 | 0.504 | 0.003 | 0.130 |
| GPR84-Tango | −0.245 | −1.284 | −1.562 | −0.320 | −0.221 | 0.123 | −0.014 | 0.277 |
| GPR85-Tango | −0.115 | −0.323 | −0.700 | −0.325 | −0.445 | 0.167 | 0.193 | −0.062 |
| GPR87-Tango | −0.030 | −1.054 | −1.050 | −0.060 | −0.208 | −0.058 | 0.370 | 0.029 |
| GPR88-Tango | −0.063 | −0.197 | −0.298 | 0.176 | −0.017 | 0.205 | 0.184 | 0.053 |
| GPR97-Tango | −0.479 | −0.263 | −0.553 | −0.050 | −0.156 | 0.041 | −0.042 | −0.002 |
| GPRC5A-Tango | −0.289 | −1.452 | −1.362 | 0.117 | 0.030 | −0.121 | −0.076 | −0.065 |
| GPRC5B-Tango | −0.169 | −1.354 | −0.973 | 0.005 | −0.069 | 0.195 | −0.019 | 0.295 |
| GPRC5C-Tango | −0.180 | −0.621 | −0.376 | 0.162 | 0.132 | 0.343 | −0.145 | 0.077 |
| GPRC5D-Tango | −0.363 | −0.487 | −1.119 | −0.240 | −0.105 | −0.095 | −0.123 | 0.038 |
| GPRC6A-Tango | −0.291 | −0.627 | −1.784 | −0.409 | −0.464 | 0.039 | −0.074 | 0.050 |
| GRM1-Tango | −0.301 | −0.662 | −1.549 | −0.590 | −0.360 | 0.013 | −0.439 | 0.073 |
| GRM2-Tango | −0.447 | −0.339 | −0.826 | −0.081 | −0.263 | 0.024 | −0.400 | 0.064 |
| GRM4-Tango | −0.431 | −0.955 | −1.206 | 0.554 | 0.545 | 0.642 | −0.024 | 0.609 |
| GRM5-Tango | −0.257 | −0.908 | −1.191 | −0.075 | 0.008 | −0.111 | −0.455 | 0.019 |
| GRM6-Tango | −0.577 | −0.895 | −1.242 | −0.481 | −0.269 | 0.032 | −0.216 | 0.000 |
| GRM7-Tango | −0.837 | −1.306 | −2.074 | −1.269 | −0.812 | −0.374 | −0.497 | −0.446 |
| GRM8-Tango | −0.278 | −0.877 | −1.908 | −0.171 | −0.222 | −0.146 | −0.425 | −0.017 |
| GRPR-Tango | −0.209 | −0.829 | −0.773 | 0.478 | 0.508 | 0.414 | −0.313 | 0.016 |
| HCA1-Tango | 0.151 | −1.055 | −0.606 | 0.034 | 0.112 | 0.309 | −0.171 | 0.160 |
| HCA2-Tango | 0.539 | −1.132 | −0.880 | −0.172 | −0.181 | 0.375 | 0.479 | 0.726 |
| HCA3-Tango | −0.069 | −0.957 | −0.564 | −0.332 | −0.277 | 0.584 | 0.108 | 0.344 |
| HCTR1-Tango | −0.033 | −0.265 | −0.874 | 0.434 | 0.000 | −0.038 | 0.261 | 0.037 |
| HCTR2-Tango | 0.117 | −0.585 | −0.082 | 0.623 | 0.623 | 0.044 | −0.376 | 0.170 |
| HRH1-Tango | 0.330 | −1.768 | −1.611 | −0.073 | −0.081 | 0.417 | 0.902 | 0.521 |
| HRH2-Tango | 0.469 | 0.123 | −0.554 | −0.068 | −0.034 | 0.356 | 0.464 | 0.300 |
| HRH3-Tango | 0.505 | −0.067 | −0.798 | −0.108 | 0.000 | 0.567 | 1.007 | 0.345 |
| HRH4-Tango | 1.046 | −0.711 | −0.883 | −0.254 | −0.231 | 0.127 | 1.023 | 0.052 |
| HTR1A-Tango | 0.323 | −0.363 | −0.976 | 0.138 | 0.172 | 0.106 | 0.393 | 0.262 |
| HTR1B-Tango | 0.117 | −1.364 | −1.453 | −0.273 | −0.217 | 0.112 | 0.504 | 0.330 |
| HTR1D-Tango | 0.038 | −1.134 | −1.039 | −0.131 | −0.251 | 0.205 | 0.259 | 0.124 |
| HTR1E-Tango | 0.177 | −1.172 | −1.706 | 0.131 | 0.113 | 0.368 | 0.549 | 0.547 |
| HTR1F-Tango | −0.369 | −1.405 | −1.024 | 0.408 | 0.081 | −0.031 | −0.423 | −0.582 |
| HTR2A-Tango | −0.314 | −0.949 | −0.824 | 0.045 | −0.012 | 0.127 | −0.111 | 0.141 |
| HTR2B-Tango | −0.084 | −2.261 | −1.518 | −0.133 | −0.255 | 0.024 | 0.261 | 0.223 |
| HTR2C-Tango | −0.066 | −0.911 | −0.755 | 0.138 | 0.088 | 0.143 | −0.094 | 0.165 |
| HTR4-Tango | −0.443 | −1.132 | −0.358 | 0.249 | 0.076 | 0.570 | 0.460 | 0.098 |
| HTR5-Tango | −0.150 | −2.148 | −1.510 | 0.394 | 0.216 | 0.339 | 0.141 | 0.391 |
| HTR6-Tango | 0.015 | −0.545 | −0.957 | 0.220 | 0.145 | 0.506 | 0.278 | 0.221 |
| HTR7-Tango | −0.019 | −1.227 | −1.084 | 0.592 | 0.572 | 0.007 | −0.183 | −0.372 |
| KISSPEPTIN-R- | 0.214 | −0.347 | −0.355 | 0.000 | 0.106 | 0.041 | 0.075 | −0.310 |
| Tango | | | | | | | | |
| LHCGR-Tango | −0.325 | −1.178 | −1.429 | 0.038 | 0.038 | 0.334 | 0.128 | 0.175 |
| LPAR1-Tango | 0.036 | −1.827 | −0.738 | 0.198 | 0.265 | 0.530 | 0.531 | 0.609 |
| LPAR2-Tango | −0.241 | −0.892 | −0.881 | −0.289 | −0.262 | 0.538 | 0.353 | 0.263 |
| LPAR4-Tango | −0.263 | −1.788 | −1.153 | 0.422 | 0.469 | 0.357 | 0.122 | 0.434 |
| LPAR5-Tango | −0.425 | −0.862 | −0.932 | 0.132 | 0.284 | 0.604 | 0.151 | 0.493 |
| LPAR6-Tango | 0.180 | −2.171 | −1.307 | 0.636 | 0.791 | 0.468 | 0.333 | 0.459 |
| LTB4R-Tango | −0.188 | −0.526 | −0.649 | 0.601 | 0.323 | 0.316 | 0.301 | 0.284 |
| LTB4R2B-Tango | 0.037 | −1.157 | −0.973 | 0.424 | 0.144 | 0.423 | −0.079 | 0.126 |
| MAS1-Tango | 0.068 | −1.003 | −0.952 | −0.062 | −0.058 | −0.066 | 0.255 | 0.084 |
| MAS1L-Tango | 0.040 | −1.217 | −1.356 | 0.064 | 0.201 | 0.006 | 0.001 | −0.022 |
| MC1R-Tango | 0.175 | −0.165 | −0.270 | −0.006 | 0.306 | 0.069 | 0.153 | −0.039 |
| MC2R-Tango | −0.206 | −0.283 | 0.255 | 0.514 | 0.316 | 0.339 | 0.052 | 0.130 |
| MC3R-Tango | −0.149 | −1.105 | −0.475 | 0.916 | 0.776 | 0.938 | 0.154 | 0.648 |
| MC4R-Tango | −0.429 | −0.449 | −0.642 | −0.074 | 0.324 | 0.566 | 0.359 | 0.559 |
| MC5R-Tango | −0.079 | −1.545 | −1.523 | −0.058 | 0.634 | 0.060 | 0.663 | 0.472 |
| MCHR1-Tango | −0.492 | −1.100 | −2.041 | 0.368 | 0.498 | 0.041 | 0.089 | −0.053 |
| MCHR2-Tango | 0.000 | −0.627 | −0.807 | 0.000 | 0.391 | 0.093 | −0.298 | −0.150 |
| MLNR-Tango | 0.135 | −0.011 | −0.404 | 0.136 | −0.206 | −0.024 | 0.129 | −0.048 |
| MRGPRD-Tango | −0.232 | −1.672 | −1.310 | −0.037 | −0.168 | 0.073 | 0.320 | 0.184 |
| MRGPRE-Tango | 0.139 | −0.218 | −0.494 | −0.196 | −0.189 | 0.169 | 0.349 | 0.112 |
| MRGPRF-Tango | −0.291 | −1.021 | −0.639 | −0.152 | −0.023 | 0.128 | 0.435 | 0.189 |
| MRGPRG-Tango | 0.025 | −0.631 | −1.155 | −0.171 | −0.066 | 0.071 | 0.216 | −0.195 |
| MRGPRX1-Tango | −0.457 | −1.484 | −1.645 | 0.290 | 0.215 | 0.231 | −0.026 | 0.193 |
| MRGPRX2-Tango | −0.319 | −1.371 | −1.219 | 0.127 | 0.206 | 0.127 | 0.194 | −0.004 |
| MRGPRX3-Tango | −0.345 | −0.457 | −0.906 | 0.000 | −0.019 | 0.423 | −0.403 | −0.296 |
| MRGPRX4-Tango | −1.261 | −1.965 | −2.479 | −0.372 | −0.483 | −0.213 | −0.717 | 0.003 |
| MTNR1A-Tango | −0.941 | −1.577 | −1.487 | −0.380 | −0.178 | 0.319 | 0.340 | 0.640 |
| MTNR1B-Tango | −0.895 | −1.503 | −1.391 | −0.419 | 0.049 | 0.272 | −0.017 | 0.351 |
| NMBR-Tango | −0.474 | −0.903 | 0.000 | 0.077 | 0.284 | 0.603 | 0.115 | 0.202 |
| NMUR1-Tango | −0.475 | −0.861 | −1.667 | −0.340 | −0.082 | 0.202 | −0.508 | −0.341 |
| NMUR2-Tango | −1.129 | −1.723 | −1.867 | −0.016 | 0.130 | 0.643 | 0.217 | 0.655 |
| NPBW1-Tango | 0.156 | 0.263 | −0.241 | 0.310 | 0.260 | 0.064 | −0.429 | −0.573 |
| NPBW2-Tango | 0.027 | −1.131 | −1.162 | −0.934 | −0.891 | −1.005 | −0.683 | −0.792 |
| NPFF1-Tango | 0.166 | 0.189 | −0.447 | 0.235 | −0.308 | 0.824 | 0.310 | −0.142 |
| NPFF2-Tango | 0.118 | −1.720 | −0.839 | 0.016 | 0.189 | 0.656 | −0.054 | 0.411 |
| NPS-Tango | −0.336 | −1.130 | −0.972 | 0.157 | 0.015 | −0.035 | −0.780 | −0.086 |
| NPY1R-Tango | 0.174 | −1.267 | −0.680 | 0.120 | 0.196 | 0.496 | −0.369 | −0.045 |
| NPY2R-Tango | −0.005 | −0.401 | −1.169 | 0.124 | 0.200 | 0.271 | −0.347 | −0.167 |
| NPY4R-Tango | −0.085 | −0.398 | −0.823 | −0.412 | −0.051 | −0.038 | −0.191 | −0.290 |
| NPY5R-Tango | −0.185 | −0.350 | −0.716 | −0.415 | −0.312 | 0.280 | 0.000 | 0.239 |
| NTSR1-Tango | −0.250 | −1.746 | −1.160 | −0.866 | −0.821 | 0.056 | 0.213 | 0.139 |
| NTSR2-Tango | −0.156 | −1.088 | −0.260 | 0.010 | −0.450 | 0.057 | −0.134 | −0.311 |
| OPN3-Tango | −0.138 | −0.197 | 0.000 | 0.110 | 0.229 | 0.602 | 0.472 | −0.053 |
| OPN5-Tango | −0.897 | −1.889 | −1.016 | −0.683 | −0.506 | 0.167 | 0.281 | 0.806 |
| OPRD1-Tango | −0.835 | −1.169 | −0.916 | −0.670 | −0.584 | 0.094 | −0.455 | 0.183 |
| OPRK1-Tango | 0.412 | −1.115 | −0.868 | 0.436 | 0.720 | 0.567 | 0.016 | 0.315 |
| OPRL1-Tango | 0.304 | −0.634 | −0.953 | 0.065 | −0.164 | 0.046 | 0.080 | −0.334 |
| OPRM1-Tango | −0.309 | −0.394 | −1.197 | −0.198 | −0.334 | 0.320 | −0.275 | −0.285 |
| OXER1-Tango | 0.287 | −1.190 | −0.747 | −0.034 | 0.007 | 0.229 | 0.117 | 0.186 |
| OXGR1-Tango | −0.786 | −1.726 | −1.236 | −0.259 | −0.412 | 0.077 | −0.299 | 0.130 |
| OXTR-Tango | −0.028 | −0.171 | 0.059 | −0.441 | −0.094 | 0.436 | 0.225 | −0.050 |
| P2RY1-Tango | 0.230 | −1.894 | −1.362 | −0.680 | −0.794 | −0.078 | 0.162 | 0.232 |
| P2RY10-Tango | −0.282 | −0.770 | −0.687 | −0.318 | −0.467 | 0.481 | 0.050 | 0.160 |
| P2RY11-Tango | −0.327 | −1.059 | −0.556 | −0.250 | −0.232 | 0.574 | 0.010 | −0.050 |
| P2RY12-Tango | −1.153 | −1.061 | −0.577 | −0.008 | −0.262 | 0.884 | −0.013 | 1.407 |
| P2RY13-Tango | −0.164 | −1.172 | −1.387 | −0.221 | −0.320 | 0.187 | 0.273 | 0.385 |
| P2RY14-Tango | 0.059 | −1.121 | −1.345 | −0.348 | −0.380 | 0.102 | 0.270 | 0.235 |
| P2RY2-Tango | −0.420 | −1.286 | −1.087 | −0.165 | −0.421 | 0.251 | 0.182 | 0.353 |
| P2RY4-Tango | −0.366 | −1.382 | −1.663 | −0.172 | −0.161 | 0.396 | 0.154 | 0.484 |
| P2RY6-Tango | −0.189 | −1.290 | −1.133 | −0.450 | −0.496 | 0.269 | 0.151 | 0.362 |
| P2RY8-Tango | −0.157 | −1.207 | −1.483 | −0.219 | −0.073 | 0.205 | −0.020 | 0.092 |
| PK1-Tango | 0.036 | −0.487 | −0.664 | 0.031 | 0.482 | 0.107 | −0.155 | −0.304 |
| PK2-Tango | −0.316 | −0.354 | −0.152 | −0.317 | 0.044 | 0.050 | −0.118 | 0.109 |
| PRRP-Tango | −0.085 | −0.478 | −0.195 | −0.312 | −0.205 | 0.109 | 0.058 | 0.359 |
| PTAFR-Tango | 0.030 | −1.465 | −0.937 | −0.531 | −0.672 | 0.092 | 0.380 | 0.447 |
| PTGDR-Tango | −0.433 | −1.025 | −0.607 | −0.523 | −0.466 | 0.295 | 0.047 | 0.113 |
| PTGER1-Tango | −0.053 | −0.485 | −0.267 | 0.482 | 0.261 | 0.408 | −0.033 | 0.556 |
| PTGER2-Tango | −0.067 | −0.531 | −0.564 | −0.202 | 0.202 | 0.721 | 0.661 | 0.251 |
| PTGER3-Tango | −0.263 | −1.393 | −0.819 | 0.087 | 0.316 | 0.284 | 0.340 | 0.463 |
| PTGER4-Tango | 0.354 | −0.831 | −0.987 | 0.927 | 0.886 | 0.557 | 0.573 | 0.577 |
| PTGFR-Tango | −0.489 | −1.028 | −0.985 | 0.345 | 0.740 | 0.770 | 0.423 | 0.668 |
| PTGIR-Tango | −0.225 | −0.280 | −1.163 | 0.048 | −0.050 | −0.272 | −0.420 | −0.722 |
| PTH1R-Tango | −0.226 | −0.436 | −1.228 | 0.029 | 0.097 | 0.499 | 0.435 | 0.201 |
| PTH2R-Tango | −0.500 | −0.892 | −1.586 | −0.403 | −0.231 | 0.387 | −0.009 | 0.327 |
| QRFP-Tango | −0.418 | −0.408 | −0.868 | −0.398 | −0.322 | 0.187 | −0.174 | 0.187 |
| RXFP1-Tango | −0.231 | −1.416 | −1.188 | −0.042 | −0.763 | 0.148 | −0.147 | −0.129 |
| RXFP2-Tango | −0.468 | −1.601 | −1.842 | −0.338 | −0.245 | 0.193 | −0.063 | 0.404 |
| RXFP3-Tango | −0.784 | −0.544 | −1.527 | −0.212 | −0.372 | −0.085 | −0.264 | −0.270 |
| RXFP4-Tango | −0.535 | −0.555 | −1.076 | −0.772 | 0.000 | −0.085 | −0.085 | −0.063 |
| S1PR1-Tango | −0.619 | −0.758 | 0.955 | −0.352 | −0.492 | 0.903 | 0.850 | 0.798 |
| S1PR2-Tango | −0.322 | −1.517 | −0.255 | 0.173 | −0.032 | 0.481 | 0.388 | 0.423 |
| S1PR3-Tango | −0.510 | −1.042 | −0.245 | 0.242 | 0.080 | 0.754 | 0.123 | 0.485 |
| S1PR4-Tango | −1.062 | −1.944 | −0.368 | −0.797 | −0.813 | −0.125 | 0.029 | 0.267 |
| S1PR5-Tango | −0.232 | −0.074 | −0.812 | −0.189 | −0.778 | 0.258 | −0.315 | −0.289 |
| SCTR-Tango | −0.310 | −1.000 | −0.243 | −0.255 | 0.016 | −0.023 | 0.159 | 0.054 |
| SSTR1-Tango | −0.422 | −1.107 | −1.507 | 0.503 | 0.395 | 0.720 | 0.160 | 0.698 |
| SSTR2-Tango | −0.374 | −0.986 | −0.247 | 0.447 | 0.318 | 0.422 | −0.474 | 0.000 |
| SSTR3-Tango | 0.216 | −0.553 | −0.213 | 0.118 | 0.468 | 0.435 | 0.200 | −0.180 |
| SSTR4-Tango | −0.193 | −1.165 | −1.603 | 0.405 | 0.405 | 0.165 | −0.140 | 0.369 |
| SSTR5-Tango | 1.335 | −0.100 | −0.428 | 1.085 | 1.229 | 0.258 | 0.504 | −0.029 |
| SUCNR1-Tango | 3.978 | 1.204 | 0.480 | 0.614 | 0.449 | −0.119 | 3.054 | −0.547 |
| TA1-Tango | −0.122 | −0.993 | −0.495 | 0.441 | 0.223 | 0.273 | −0.140 | 0.247 |
| TAAR2-Tango | −0.239 | −0.922 | −0.584 | 0.101 | 0.101 | 0.150 | 0.071 | 0.205 |
| TAAR5-Tango | −0.081 | −0.328 | −1.137 | −0.429 | −0.297 | −0.462 | −0.242 | −0.473 |
| TAAR6-Tango | −0.380 | −0.864 | −1.487 | −0.189 | −0.220 | −0.489 | −0.559 | −0.354 |
| TAAR8-Tango | −0.293 | −0.912 | −1.473 | −0.671 | −0.411 | 0.100 | −0.131 | −0.197 |
| TAAR9-Tango | −0.167 | −0.880 | −1.384 | −0.311 | −0.381 | 0.149 | −0.024 | 0.169 |
| TACR1-Tango | −0.211 | −0.839 | −1.018 | −0.190 | −0.157 | 0.229 | 0.570 | 0.133 |
| TACR2-Tango | −0.046 | −0.745 | −0.209 | 0.017 | −0.262 | 0.298 | 0.356 | 0.397 |
| TACR3-Tango | −0.449 | −0.909 | −0.766 | 0.067 | 0.000 | 0.259 | −0.066 | 0.194 |
| TBXA2R-Tango | −0.225 | −0.413 | −0.340 | −0.064 | 0.099 | 0.317 | 0.045 | −0.191 |
| TSHR-Tango | −0.578 | −1.170 | −1.804 | −0.065 | 0.187 | −0.112 | −0.825 | −0.178 |
| UTS2R-Tango | −0.269 | −0.310 | −0.836 | −0.020 | 0.184 | −0.133 | −0.309 | −0.647 |
| VIPR1-Tango | −0.505 | −1.528 | −1.306 | −0.104 | 0.489 | 0.177 | 0.403 | 0.758 |
| VIPR2-Tango | 1.353 | −0.050 | 0.467 | 0.298 | 0.649 | 0.531 | 1.343 | 1.464 |

| | | Bifidobac- | Bifidobac- | Bifidobac- | Bifidobac- | Bifidobac- | Bifidobac- | Bifidobac- |
|---|---|---|---|---|---|---|---|---|
| PRESTO-Tango | Eggerthela | terium | terium | terium | terium | terium | terium | terium |
| with GMM | lenta | spp | UC-1# | UC-2# | UC-3# | UC-4# | UC-5# | UC-6# |
| ADCYAP1R1- | 0.114 | 0.510 | 0.141 | −0.456 | 0.082 | 0.438 | −0.302 | 0.213 |
| Tango | | | | | | | | |
| ADORA1-Tango | −0.333 | 0.423 | −0.340 | −0.863 | −0.291 | 0.356 | 0.160 | −0.091 |
| ADORA2A-Tango | −0.663 | 0.694 | −0.589 | −0.223 | 0.076 | 0.309 | −0.343 | −0.256 |
| ADORA2B-Tango | −0.239 | 0.093 | −0.057 | −0.258 | −0.268 | −0.079 | −0.418 | 0.024 |
| ADORA3-Tango | −0.077 | 0.905 | 0.518 | −0.248 | 0.383 | 0.508 | 0.380 | 0.387 |
| ADRA1A-Tango | −0.211 | 0.554 | 0.029 | −0.188 | −0.005 | 0.214 | −0.205 | −0.120 |
| ADRA1B-Tango | −0.371 | 0.651 | −0.009 | −0.620 | −0.676 | 0.263 | −0.232 | −0.853 |
| ADRA1D-Tango | −0.376 | 0.423 | −0.309 | −0.329 | −0.691 | 0.335 | −0.443 | −0.834 |
| ADRA2A-Tango | 0.033 | 0.182 | 0.964 | 0.427 | 0.302 | 0.000 | −0.533 | 0.745 |
| ADRA2B-Tango | 0.000 | −0.306 | −0.144 | −0.178 | 0.000 | −0.160 | −1.178 | −0.515 |
| ADRA2C-Tango | −0.291 | 0.733 | 0.505 | −0.115 | 0.335 | 0.000 | −1.133 | −0.258 |
| ADRB1-Tango | 0.200 | 1.124 | 0.465 | 0.082 | −0.292 | −0.122 | −0.577 | 0.551 |
| ADRB2-Tango | −0.234 | 0.555 | −0.195 | −0.228 | 0.065 | −0.030 | −0.360 | −0.234 |
| ADRB3-Tango | 0.023 | 0.684 | 0.057 | 0.146 | −0.411 | −0.111 | −0.136 | 0.225 |
| AGTR1-Tango | −0.091 | 0.256 | −0.179 | −0.306 | −0.627 | 0.349 | −0.331 | −0.131 |
| AGTR2-Tango | 0.238 | 0.570 | −0.148 | −0.095 | −0.133 | 0.030 | −0.494 | −0.065 |
| APJ-Tango | −0.168 | 0.486 | −0.413 | −0.010 | 0.131 | −0.154 | −0.073 | 0.164 |
| AVPR1A-Tango | −0.499 | 0.682 | 0.262 | 0.092 | 0.323 | 0.267 | −0.124 | 0.428 |
| AVPR1B-Tango | −0.055 | 0.318 | −0.339 | −0.255 | −0.207 | 0.166 | −0.180 | −0.062 |
| AVPR2-Tango | 0.228 | 0.466 | 0.109 | −0.111 | −0.331 | 0.297 | −0.230 | 0.496 |
| BB3-Tango | 0.287 | 0.371 | −0.815 | −0.656 | −0.340 | 0.348 | −0.159 | −0.539 |
| BDKBR1-Tango | 0.037 | 0.444 | −0.428 | −0.256 | −0.524 | 0.321 | −0.387 | 0.115 |
| BDKBR2-Tango | 0.430 | 0.974 | −0.259 | −0.217 | −0.442 | 0.261 | 0.016 | 0.882 |
| C3AR1-Tango | 0.438 | 0.280 | −0.710 | −0.813 | −0.533 | 0.280 | −0.265 | 0.239 |
| C5A-Tango | −0.339 | 0.723 | −0.023 | 0.230 | 0.313 | 0.463 | −0.357 | 0.286 |
| CALCRb-Tango | −0.348 | 0.413 | 0.090 | −0.037 | −0.129 | 0.152 | −0.628 | 0.177 |
| CALCRL-Tango | −0.250 | 0.246 | 0.009 | −0.006 | 0.045 | 0.128 | −0.341 | −0.362 |
| CASR-Tango | −0.569 | 0.850 | −0.054 | −0.338 | 0.110 | 0.482 | 0.133 | −0.662 |
| CCKAR-Tango | 0.239 | 0.856 | −0.203 | −0.166 | 0.134 | 0.264 | 0.110 | 0.145 |
| CCKBR-Tango | −0.031 | 0.621 | −0.466 | −0.091 | −0.303 | 0.052 | −0.334 | −0.006 |
| CCR10-Tango | −0.162 | −0.084 | −0.269 | 0.025 | −0.374 | −0.208 | −0.459 | 0.152 |
| CCR2-Tango | −0.206 | 0.456 | −0.228 | −0.346 | −0.231 | 0.200 | −0.248 | 0.189 |
| CCR3-Tango | −0.188 | 0.660 | 0.063 | −0.596 | −0.311 | 0.561 | 0.161 | 0.238 |
| CCR4-Tango | −0.379 | 0.820 | −0.420 | −0.242 | −0.346 | 0.474 | 0.000 | −0.417 |
| CCR5-Tango | −0.339 | 0.832 | −0.461 | −0.062 | 0.210 | 0.298 | −0.443 | −0.100 |
| CCR6-Tango | −0.270 | 0.706 | −0.330 | −0.470 | −0.420 | 0.030 | −0.315 | 0.107 |
| CCR7-Tango | −0.125 | 0.452 | −0.727 | −0.115 | −0.608 | 0.137 | −0.022 | −0.619 |
| CCR8-Tango | −0.767 | 0.044 | −0.395 | −0.103 | −0.263 | −0.528 | −1.062 | −0.360 |
| CCRL2-Tango | −0.631 | 1.163 | −0.341 | −0.213 | −0.263 | 0.193 | −0.155 | −0.077 |
| CD97-Tango | −0.426 | 0.368 | −0.680 | −0.459 | −0.404 | 0.280 | −0.284 | −0.968 |
| CHRM1-Tango | −0.604 | 0.291 | −0.176 | 0.155 | 0.234 | 0.210 | −0.814 | −0.284 |
| CHRM2-Tango | 0.164 | 0.612 | −0.485 | −0.126 | 0.568 | 0.090 | −0.120 | −0.195 |
| CHRM3-Tango | −0.153 | 0.501 | −0.164 | 0.052 | 0.173 | 0.232 | −0.277 | −0.493 |
| CHRM4-Tango | −0.129 | 0.432 | −0.115 | −0.076 | −0.086 | 0.175 | −0.391 | −0.142 |
| CHRM5-Tango | 0.070 | 0.725 | −0.334 | 0.003 | 0.297 | 0.260 | −0.163 | 0.025 |
| CMKLR1-Tango | −0.190 | 0.619 | −0.141 | −0.033 | −0.071 | 0.232 | −0.174 | 0.479 |
| CNR1-Tango | −0.048 | 0.657 | −0.369 | −0.324 | 0.105 | 0.097 | −0.311 | −0.213 |
| CNR2-Tango | −0.201 | 0.536 | −0.496 | −0.412 | −0.334 | 0.058 | −0.369 | 0.132 |
| CRHR1-Tango | −0.040 | 0.270 | 0.057 | −0.722 | −0.044 | 0.170 | −0.373 | −0.040 |
| CRHR2-Tango | −0.334 | 0.557 | −0.178 | −0.252 | 0.318 | −0.232 | −0.579 | 0.230 |
| CX3C1-Tango | 0.447 | 0.537 | −0.985 | −0.255 | −0.520 | 0.026 | −0.072 | 0.318 |
| CXCR1-Tango | −0.875 | 0.475 | 0.038 | 0.354 | 0.354 | 0.361 | −0.370 | −0.064 |
| CXCR2-Tango | −0.152 | 0.560 | 0.090 | −0.273 | 0.074 | 0.248 | −0.177 | 0.244 |
| CXCR3-Tango | 0.127 | 0.486 | −0.138 | 0.357 | 0.130 | 0.040 | −0.453 | 0.127 |
| CXCR4-Tango | 0.424 | 0.995 | −0.098 | 0.124 | 0.076 | 0.627 | 0.033 | 0.221 |
| CXCR5-Tango | 0.223 | 0.703 | 0.184 | −0.263 | −0.129 | 0.303 | −0.072 | 0.284 |
| CXCR6-Tango | 0.500 | −0.209 | −0.260 | −0.079 | −0.263 | −0.093 | −0.335 | 0.455 |
| CXCR7-Tango | 0.035 | −0.155 | −0.642 | −0.635 | −0.334 | 0.193 | −0.372 | −0.480 |
| CYSLTR1-Tango | −0.078 | 0.167 | −0.414 | −0.051 | 0.281 | 0.297 | −0.169 | −0.232 |
| CYSLTR2-Tango | −0.620 | 0.496 | 0.265 | 0.463 | 0.071 | 0.451 | −1.212 | −0.101 |
| DRD1-Tango | −0.342 | 0.743 | −0.315 | 0.240 | 0.155 | 0.875 | −0.605 | 0.362 |
| DRD2-Tango | −0.913 | 0.952 | 0.170 | 1.420 | −0.293 | 1.091 | 0.049 | −0.016 |
| DRD3-Tango | −1.303 | 0.733 | −0.503 | 0.876 | 0.098 | 1.628 | −0.360 | 0.302 |
| DRD4-Tango | −0.526 | 1.186 | −0.369 | 0.521 | 0.305 | 1.328 | 0.594 | −0.328 |
| DRD5-Tango | −0.234 | −0.293 | −0.687 | −0.203 | −0.378 | −0.420 | −0.984 | 0.154 |
| EDNRA-Tango | 0.037 | 0.696 | −0.390 | −0.448 | 0.032 | 0.294 | −0.055 | 0.242 |
| EDNRB-Tango | −0.306 | 0.349 | −0.571 | −0.114 | −0.160 | 0.157 | −0.078 | −0.012 |
| ELTD1-Tango | −0.438 | 0.254 | −0.978 | −0.665 | −0.028 | 0.326 | 0.076 | −0.608 |
| F2R-Tango | −0.192 | 0.497 | −0.594 | −0.501 | −0.089 | 0.064 | −0.343 | 0.392 |
| F2RL1-Tango | −0.165 | 0.445 | −0.553 | −0.371 | −0.039 | 0.153 | −0.316 | 0.018 |
| F2RL2-Tango | −0.118 | 0.854 | −0.052 | −0.537 | −0.089 | 0.474 | 0.101 | −0.114 |
| F2RL3-Tango | −0.202 | 0.644 | −0.484 | −0.740 | −0.242 | 0.318 | −0.201 | −0.194 |
| FFA1-Tango | 0.118 | 0.671 | −0.256 | −0.101 | 0.172 | 0.502 | 0.720 | 0.219 |
| FFA2-Tango | −0.067 | 0.641 | −0.025 | 0.095 | 0.316 | 0.450 | 0.251 | 0.164 |
| FFA3-Tango | 0.210 | 0.817 | −0.022 | −0.244 | 0.034 | 0.286 | 0.167 | 0.256 |
| FPR1-Tango | 0.203 | −0.127 | −0.609 | −0.113 | 0.197 | 0.988 | −0.283 | −0.587 |
| FPR2-Tango | 0.106 | 0.571 | −0.200 | 0.095 | 0.278 | 0.369 | −0.072 | 0.153 |
| FPR3-Tango | 0.056 | 0.478 | −0.280 | 0.018 | 0.142 | 0.324 | 0.043 | −0.093 |
| FSHR-Tango | 0.324 | 0.569 | −0.420 | −0.081 | 0.396 | 0.556 | 0.126 | −0.020 |
| GABBR1-Tango | −0.313 | 0.320 | −0.103 | −0.033 | −0.109 | 0.244 | −0.596 | 0.008 |
| GAL1-Tango | 0.073 | −0.044 | −0.790 | 0.267 | −0.670 | −0.328 | −0.184 | −0.399 |
| GAL2-Tango | −0.071 | 0.721 | −0.234 | 0.265 | 0.428 | 0.721 | −0.420 | 0.089 |
| GAL3-Tango | −0.354 | −0.057 | −0.303 | −0.870 | 0.194 | −0.136 | −0.355 | 0.219 |
| GCGR-Tango | −0.050 | 0.431 | −0.240 | −0.043 | 0.339 | 0.377 | −0.040 | 0.347 |
| GHRHR-Tango | 0.257 | 0.214 | −0.422 | −0.155 | 0.084 | −0.063 | −0.751 | 0.738 |
| GHSR-Tango | 0.159 | 0.488 | −0.636 | −0.667 | −0.435 | 0.343 | −0.110 | 0.533 |
| GIPR-Tango | −0.072 | −0.090 | −0.724 | −0.567 | −0.244 | −0.141 | −0.861 | 0.519 |
| GLP1R-Tango | −0.063 | 0.508 | −0.451 | −0.651 | 0.035 | 0.218 | 0.390 | 0.110 |
| GLP2R-Tango | −0.247 | 0.336 | −0.158 | −0.009 | 0.107 | 0.125 | −0.139 | 0.324 |
| GNRHR-Tango | −0.106 | 0.170 | 0.086 | −0.111 | 0.000 | 0.347 | −0.229 | −0.266 |
| GPBA-Tango | 0.206 | 0.165 | −0.384 | −0.094 | 0.108 | 0.000 | −0.339 | −0.304 |
| GPER-Tango | −0.178 | 0.131 | −0.113 | −0.225 | −0.225 | 0.476 | −0.461 | 0.128 |
| GPR1-Tango | 0.068 | 0.382 | −0.392 | −0.075 | −0.071 | 0.386 | −0.317 | −0.280 |
| GPR101-Tango | −0.008 | 0.181 | −0.506 | 0.157 | 0.131 | 0.275 | −0.175 | 0.221 |
| GPR110-Tango | 0.019 | 0.365 | −0.312 | −0.723 | −0.206 | 0.251 | −0.332 | −0.358 |
| GPR111-Tango | −0.191 | 0.193 | −0.194 | −0.643 | −0.586 | 0.093 | −0.636 | −0.163 |
| GPR113-Tango | −0.262 | 0.141 | −0.348 | −0.306 | −0.057 | 0.107 | −0.180 | −0.164 |
| GPR114-Tango | 0.018 | −0.024 | −0.015 | 0.141 | 0.230 | −0.234 | −0.491 | −0.087 |
| GPR115-Tango | −0.104 | 0.352 | −0.030 | −0.547 | −0.133 | 0.263 | 0.009 | −0.331 |
| GPR116-Tango | −0.293 | 0.263 | 0.009 | −0.531 | −0.419 | 0.288 | −0.089 | −0.366 |
| GPR119-Tango | −0.225 | 0.280 | 0.066 | 0.028 | −0.119 | 0.139 | −0.473 | −0.091 |
| GPR12-Tango | 0.134 | 0.566 | −0.253 | 0.163 | −0.299 | 0.446 | −0.347 | 0.342 |
| GPR120-Tango | −0.144 | 0.260 | 0.057 | −0.397 | −0.256 | 0.349 | −0.503 | −0.293 |
| GPR123-Tango | 0.005 | 0.604 | 0.238 | −0.394 | 0.432 | 0.354 | −0.185 | 0.296 |
| GPR124-Tango | −0.368 | 0.577 | 0.172 | −0.824 | −1.003 | 0.442 | 0.117 | −0.778 |
| GPR125-Tango | −0.442 | 0.060 | 0.017 | −0.875 | −0.919 | −0.017 | −0.310 | −0.519 |
| GPR126-Tango | −0.210 | 0.275 | −0.199 | −0.678 | −0.598 | −0.005 | −0.439 | 0.192 |
| GPR132-Tango | −0.402 | 0.821 | 0.094 | −0.906 | −0.169 | 0.525 | −0.015 | −0.204 |
| GPR133-Tango | −0.019 | 0.669 | −0.089 | −0.499 | −0.157 | 0.541 | −0.078 | −0.149 |
| GPR135-Tango | −0.252 | 0.078 | −0.163 | −0.458 | −0.363 | −0.097 | −0.902 | 0.583 |
| GPR141-Tango | −0.522 | 0.533 | −0.009 | −0.925 | −0.806 | 0.381 | −0.138 | 0.205 |
| GPR142-Tango | −0.573 | 0.771 | −0.382 | −0.940 | −0.549 | 0.278 | −0.115 | −0.696 |
| GPR143-Tango | −0.528 | 0.861 | 0.044 | −0.467 | 0.364 | 0.577 | 0.280 | −0.670 |
| GPR144-Tango | −0.569 | 0.465 | −0.158 | −0.477 | −0.381 | 0.485 | −0.141 | −0.786 |
| GPR146-Tango | −0.745 | 0.531 | 0.410 | −1.082 | −0.420 | 0.812 | 0.531 | 0.133 |
| GPR148-Tango | −0.477 | 0.093 | −0.333 | −0.591 | −0.951 | 0.658 | 0.341 | −0.828 |
| GPR149-Tango | −0.070 | 0.790 | 0.017 | 0.371 | 0.425 | 0.693 | 0.515 | −0.902 |
| GPR15-Tango | −0.152 | 0.726 | −0.186 | 0.501 | 0.471 | 0.692 | 0.033 | −0.444 |
| GPR150-Tango | −0.778 | 0.181 | −0.405 | −0.220 | −0.420 | 0.599 | −0.881 | 0.170 |
| GPR151-Tango | −0.478 | 0.784 | −0.087 | 0.167 | 0.361 | 0.784 | 0.260 | −0.343 |
| GPR152-Tango | −0.060 | 0.148 | 0.369 | −0.258 | 0.003 | 0.098 | −0.130 | −0.244 |
| GPR153-Tango | 0.241 | 0.149 | 0.367 | −0.191 | 0.102 | 0.239 | −0.061 | 0.000 |
| GPR156-Tango | 0.052 | −0.287 | 0.464 | −0.286 | −0.296 | −0.149 | −0.244 | 0.059 |
| GPR157-Tango | −0.066 | 0.209 | 0.173 | −0.295 | −0.338 | −0.087 | −0.407 | −0.407 |
| GPR158-Tango | −0.147 | 0.265 | 0.271 | −0.126 | 0.062 | 0.244 | −0.677 | −0.126 |
| GPR160-Tango | 0.041 | 0.686 | 0.143 | −0.137 | 0.276 | 0.377 | −0.117 | −0.266 |
| GPR161-Tango | −0.047 | 0.422 | 0.132 | −0.268 | −0.191 | 0.317 | 0.035 | −0.484 |
| GPR162-Tango | −0.197 | −0.105 | −0.239 | −0.449 | −0.330 | −0.134 | −0.471 | −0.257 |
| GPR17-Tango | −0.074 | 0.445 | 0.370 | 0.380 | 0.408 | 0.315 | 0.438 | −0.778 |
| GPR171-Tango | −0.201 | 0.706 | 0.731 | −0.081 | 0.131 | 0.474 | 0.058 | −0.420 |
| GPR173-Tango | −0.047 | −0.047 | −0.055 | −0.186 | −0.157 | 0.201 | −0.675 | 0.070 |
| GPR174-Tango | −0.254 | 1.075 | −0.027 | −0.225 | −0.425 | 0.550 | 0.152 | −0.254 |
| GPR18-Tango | −0.175 | 0.448 | −0.370 | −0.182 | −0.531 | 0.256 | −0.368 | −0.125 |
| GPR182-Tango | 0.042 | 0.770 | 0.011 | 0.074 | 0.089 | 0.531 | −0.055 | −0.506 |
| GPR183-Tango | 0.102 | 0.425 | −0.224 | −0.184 | 0.096 | 0.353 | 0.064 | 0.040 |
| GPR19-Tango | −0.032 | 0.935 | 0.139 | −0.483 | 0.138 | 0.473 | 0.219 | −0.573 |
| GPR20-Tango | 0.030 | 0.497 | 0.261 | −0.361 | −0.191 | 0.404 | 0.234 | 0.200 |
| GPR21-Tango | −0.039 | 0.714 | 0.215 | 0.361 | 0.124 | 0.087 | −0.142 | 0.104 |
| GPR22-Tango | 0.376 | 0.371 | 0.025 | −0.318 | 0.123 | 0.398 | 0.362 | −0.335 |
| GPR25-Tango | −0.179 | 0.184 | −0.099 | 0.156 | −0.373 | 0.275 | −0.082 | −0.078 |
| GPR26-Tango | 0.348 | 0.399 | −0.408 | −0.649 | −0.578 | 0.197 | −0.070 | 0.090 |
| GPR27-Tango | 0.014 | 0.484 | 0.547 | 0.225 | −0.343 | 0.214 | −0.142 | 0.125 |
| GPR3-Tango | −0.080 | 0.565 | −0.501 | 0.105 | −0.214 | 0.665 | 0.433 | 0.174 |
| GPR31-Tango | −0.098 | 0.807 | −0.467 | −0.185 | −0.467 | 0.907 | 0.205 | 0.149 |
| GPR32-Tango | 0.263 | 0.203 | −0.232 | 0.055 | 0.028 | −0.064 | −0.292 | 0.097 |
| GPR34-Tango | −0.182 | 0.500 | −0.301 | −0.447 | −0.045 | 0.404 | −0.197 | 0.009 |
| GPR35-Tango | −0.221 | 0.632 | 0.193 | −0.189 | −0.085 | 0.214 | 0.043 | 0.254 |
| GPR37-Tango | −0.179 | 0.702 | 0.068 | −0.220 | −0.291 | 0.436 | −0.021 | −0.037 |
| GPR37L1-Tango | 0.106 | 0.500 | 0.067 | −0.592 | −0.353 | 0.320 | −0.155 | −0.059 |
| GPR39-Tango | −0.177 | 0.158 | −0.186 | −0.452 | −0.436 | 0.039 | −0.496 | −0.170 |
| GPR4-Tango | −0.078 | 0.940 | 0.083 | −0.399 | −0.455 | 0.607 | 0.084 | 0.013 |
| GPR44-Tango | 0.090 | 0.310 | −0.233 | −0.404 | −0.200 | 0.222 | −0.603 | 0.279 |
| GPR45-Tango | −0.224 | 0.292 | −0.503 | −0.315 | 0.222 | 0.427 | −0.350 | 0.595 |
| GPR50-Tango | −0.216 | 1.007 | 0.592 | −0.392 | 0.109 | 0.468 | 0.345 | −0.143 |
| GPR52-Tango | −0.084 | 0.314 | 0.098 | 0.580 | −0.599 | −0.045 | −0.449 | 0.259 |
| GPR55-Tango | 0.086 | 0.812 | 0.425 | 0.274 | 0.341 | 0.396 | 0.116 | 0.125 |
| GPR56-Tango | −0.090 | 0.619 | 0.229 | −0.859 | −0.528 | 0.884 | 0.468 | −0.020 |
| GPR6-Tango | 0.044 | 1.132 | 0.375 | 0.136 | 0.058 | 0.767 | 0.233 | 0.020 |
| GPR61-Tango | −0.036 | 0.915 | 0.319 | −0.163 | −0.097 | 0.539 | −0.423 | 0.038 |
| GPR62-Tango | 0.113 | 0.873 | 0.657 | −0.455 | −0.295 | 0.407 | −0.405 | 0.340 |
| GPR63-Tango | −0.421 | −0.255 | 0.320 | 0.037 | 0.209 | 0.003 | −0.337 | −0.490 |
| GPR64-Tango | −0.064 | −0.180 | 0.166 | −0.392 | −0.020 | −0.125 | −0.346 | −0.102 |
| GPR65-Tango | 0.091 | 0.365 | 0.125 | 0.023 | 0.255 | 0.136 | −0.105 | −0.064 |
| GPR68-Tango | 0.055 | 0.124 | 0.087 | −0.571 | −0.016 | 0.319 | −0.165 | −0.120 |
| GPR75-Tango | −0.176 | 0.565 | 0.138 | 0.213 | 0.238 | 0.236 | −0.074 | −0.135 |
| GPR77-Tango | −0.153 | 0.162 | 0.016 | −0.227 | −0.059 | 0.029 | −0.745 | −0.157 |
| GPR78-Tango | −0.197 | 0.856 | 0.212 | 0.080 | −0.033 | 0.448 | −0.077 | −0.051 |
| GPR82-Tango | 0.048 | 0.500 | −0.125 | −0.551 | −0.287 | 0.047 | −0.222 | 0.084 |
| GPR83-Tango | 0.136 | 0.262 | 0.249 | −0.581 | 0.126 | 0.149 | 0.101 | −0.032 |
| GPR84-Tango | −0.033 | 0.483 | 0.020 | −0.283 | 0.013 | 0.410 | 0.190 | −0.552 |
| GPR85-Tango | −0.140 | 0.562 | 0.201 | −0.218 | −0.209 | 0.357 | 0.000 | −0.154 |
| GPR87-Tango | −0.189 | 0.378 | 0.042 | −0.029 | −0.065 | 0.445 | −0.153 | 0.051 |
| GPR88-Tango | 0.035 | 0.190 | 0.084 | 0.219 | −0.057 | 0.106 | −0.522 | 0.270 |
| GPR97-Tango | 0.144 | 0.535 | 0.209 | −0.100 | −0.190 | 0.215 | −0.402 | −0.510 |
| GPRC5A-Tango | −0.196 | 0.866 | 0.051 | −0.482 | 0.042 | 0.679 | 0.350 | −0.198 |
| GPRC5B-Tango | −0.004 | 0.888 | 0.008 | −0.168 | 0.038 | 0.578 | 0.360 | −0.539 |
| GPRC5C-Tango | −0.235 | 0.488 | 0.188 | 0.154 | 0.569 | 0.628 | 0.231 | −0.335 |
| GPRC5D-Tango | −0.047 | −0.042 | 0.230 | −0.270 | −0.229 | 0.056 | −0.345 | −0.208 |
| GPRC6A-Tango | 0.125 | 0.325 | 0.633 | −0.471 | −0.273 | 0.215 | −0.284 | −0.446 |
| GRM1-Tango | −0.006 | 0.288 | −0.042 | −0.556 | −0.466 | 0.139 | −0.360 | −0.311 |
| GRM2-Tango | −0.306 | 0.211 | 0.175 | −0.390 | −0.269 | −0.046 | −0.457 | −0.448 |
| GRM4-Tango | −0.119 | 0.879 | 0.426 | −0.524 | 0.312 | 0.703 | 0.354 | −0.356 |
| GRM5-Tango | −0.028 | −0.118 | 0.159 | −0.569 | −0.261 | −0.131 | −0.726 | −0.305 |
| GRM6-Tango | −0.427 | −0.663 | 0.131 | −0.332 | −0.358 | −0.478 | −0.264 | −0.876 |
| GRM7-Tango | −0.075 | −0.468 | 0.084 | −0.693 | −0.682 | −0.328 | −0.752 | −0.857 |
| GRM8-Tango | −0.140 | −0.255 | 0.047 | −0.534 | −0.683 | 0.007 | −0.363 | −0.921 |
| GRPR-Tango | 0.348 | 0.406 | 0.066 | −0.312 | 0.140 | 0.343 | −0.060 | 0.200 |
| HCA1-Tango | −0.141 | 0.712 | 0.191 | −0.297 | 0.198 | 0.381 | −0.089 | −0.421 |
| HCA2-Tango | −0.105 | 1.083 | 0.237 | −0.017 | 0.241 | 0.892 | 0.387 | −0.606 |
| HCA3-Tango | −0.280 | 1.059 | 0.000 | −0.339 | −0.095 | 0.842 | 0.253 | −0.560 |
| HCTR1-Tango | 0.377 | 1.012 | −0.387 | −1.033 | 0.095 | 0.350 | 0.313 | 0.317 |
| HCTR2-Tango | −0.093 | 0.722 | −0.234 | 0.078 | 0.705 | 0.377 | −0.341 | −0.497 |
| HRH1-Tango | 0.101 | 0.384 | −0.347 | −0.008 | −0.038 | 0.370 | 0.061 | −0.682 |
| HRH2-Tango | −0.080 | 0.277 | 0.193 | 0.404 | −0.187 | 0.365 | −0.086 | −0.113 |
| HRH3-Tango | −0.374 | −0.100 | 0.393 | 0.105 | −0.327 | −0.109 | −0.874 | −0.208 |
| HRH4-Tango | 0.492 | 0.393 | −0.496 | −0.345 | −0.686 | 0.280 | −0.327 | 0.221 |
| HTR1A-Tango | −0.162 | 0.974 | 0.300 | 0.051 | −0.028 | 0.827 | 0.165 | 0.000 |
| HTR1B-Tango | 0.332 | 0.813 | −0.236 | −0.259 | −0.178 | 0.598 | 0.239 | −0.397 |
| HTR1D-Tango | −0.402 | 0.654 | −0.381 | −0.094 | −0.139 | 0.480 | −0.027 | −0.221 |
| HTR1E-Tango | −0.266 | 0.742 | −0.007 | −0.206 | −0.127 | 0.454 | 0.086 | −0.264 |
| HTR1F-Tango | 0.105 | 0.619 | −0.460 | −0.168 | 0.226 | 0.435 | 0.066 | −0.478 |
| HTR2A-Tango | −0.441 | 0.377 | −0.353 | −0.195 | 0.150 | 0.177 | −0.359 | −0.874 |
| HTR2B-Tango | −0.005 | 0.928 | −0.141 | −0.213 | 0.004 | 0.607 | 0.132 | −0.766 |
| HTR2C-Tango | −0.290 | 0.318 | 0.174 | −0.283 | −0.287 | 0.073 | −0.770 | −0.703 |
| HTR4-Tango | 0.029 | 0.702 | −0.415 | 0.099 | 0.032 | 0.242 | −0.177 | 0.324 |
| HTR5-Tango | −0.195 | 0.701 | 0.000 | 0.052 | 0.295 | 0.550 | 0.070 | 0.110 |
| HTR6-Tango | −0.696 | 0.338 | −0.280 | −0.195 | −0.499 | 0.350 | −0.282 | −1.044 |
| HTR7-Tango | −0.436 | 0.888 | 0.373 | −0.430 | −0.142 | 0.299 | −0.004 | −0.419 |
| KISSPEPTIN-R- | 0.000 | −0.031 | −0.397 | 0.030 | −0.062 | −0.094 | −1.127 | 0.093 |
| Tango | | | | | | | | |
| LHCGR-Tango | −0.543 | 1.003 | −0.512 | −0.669 | −0.212 | 0.451 | 0.359 | −0.634 |
| LPAR1-Tango | 0.123 | 0.997 | −0.060 | −0.244 | 0.392 | 0.735 | 0.280 | −0.140 |
| LPAR2-Tango | −0.539 | 0.515 | −0.042 | −0.551 | −0.165 | 0.218 | −0.352 | −0.153 |
| LPAR4-Tango | −0.178 | 0.886 | 0.057 | −0.399 | −0.010 | 0.277 | −0.020 | −0.290 |
| LPAR5-Tango | −0.622 | 0.634 | 0.122 | −0.210 | −0.082 | 0.380 | −0.304 | −0.733 |
| LPAR6-Tango | −0.059 | 0.799 | 0.165 | −0.269 | 0.023 | 0.491 | 0.252 | −0.817 |
| LTB4R-Tango | −0.140 | 0.259 | 0.267 | −0.098 | −0.435 | 0.135 | −0.305 | −0.520 |
| LTB4R2B-Tango | −0.152 | 0.816 | −0.335 | −0.144 | 0.210 | 0.552 | 0.384 | −0.821 |
| MAS1-Tango | 0.351 | 0.387 | −0.112 | −0.293 | −0.010 | 0.274 | −0.373 | −0.288 |
| MAS1L-Tango | −0.026 | 0.274 | −0.302 | −0.325 | 0.079 | 0.256 | −0.051 | −0.751 |
| MC1R-Tango | −0.262 | 0.131 | −0.135 | 0.049 | 0.015 | 0.070 | −0.475 | −0.804 |
| MC2R-Tango | 0.041 | 0.432 | −0.057 | 0.608 | 0.666 | 0.227 | −0.537 | −0.357 |
| MC3R-Tango | −0.292 | 0.884 | −0.240 | −0.053 | 0.160 | 0.213 | −0.430 | −0.371 |
| MC4R-Tango | −0.852 | 0.681 | 0.160 | 0.087 | 0.022 | 0.647 | 0.141 | −0.325 |
| MC5R-Tango | 0.435 | 1.188 | −0.606 | −0.058 | −0.189 | 0.415 | −0.136 | 0.435 |
| MCHR1-Tango | 0.000 | −0.808 | 0.150 | −0.588 | −0.413 | −0.703 | −1.192 | −1.091 |
| MCHR2-Tango | −0.271 | −0.413 | 0.115 | −0.371 | −0.511 | −0.345 | −0.602 | −1.142 |
| MLNR-Tango | 0.085 | −0.067 | 0.341 | −0.357 | −0.532 | 0.047 | −0.272 | −0.276 |
| MRGPRD-Tango | 0.043 | 0.019 | −0.068 | −0.307 | −0.040 | 0.088 | −0.142 | −0.971 |
| MRGPRE-Tango | −0.144 | 0.056 | 0.327 | −0.125 | −0.009 | −0.071 | −0.345 | −0.118 |
| MRGPRF-Tango | 0.052 | 0.581 | −0.070 | 0.162 | 0.020 | 0.611 | −0.307 | −0.398 |
| MRGPRG-Tango | 0.050 | −0.292 | 0.053 | −0.239 | −0.075 | −0.485 | −0.642 | −0.310 |
| MRGPRX1-Tango | −0.071 | 0.064 | −0.312 | −0.801 | −0.596 | −0.417 | −0.865 | −0.720 |
| MRGPRX2-Tango | −0.653 | 0.043 | 0.065 | 0.174 | 0.352 | 0.121 | 0.052 | −1.278 |
| MRGPRX3-Tango | −0.547 | 0.246 | −0.275 | −0.383 | −0.095 | 0.034 | −0.214 | −0.426 |
| MRGPRX4-Tango | 0.182 | 0.062 | 0.012 | −0.216 | 0.091 | −0.494 | −0.021 | −1.103 |
| MTNR1A-Tango | 0.541 | 0.188 | −0.250 | −0.548 | −0.259 | 0.219 | −0.489 | −0.365 |
| MTNR1B-Tango | −0.732 | 0.410 | −0.146 | −0.096 | 0.111 | 0.054 | −0.643 | −0.845 |
| NMBR-Tango | 0.131 | 0.262 | −0.334 | 0.585 | 0.680 | 0.433 | −0.523 | −0.100 |
| NMUR1-Tango | 0.922 | 1.073 | −0.539 | −0.317 | −0.374 | 0.883 | −0.117 | 0.525 |
| NMUR2-Tango | −0.699 | 1.092 | 0.493 | −0.415 | −0.256 | 1.198 | 0.707 | −0.195 |
| NPBW1-Tango | −0.115 | 0.758 | −0.057 | −0.241 | 0.077 | 0.501 | 0.625 | 0.187 |
| NPBW2-Tango | −0.046 | 0.426 | 0.093 | −0.716 | 0.299 | 0.112 | 0.924 | −0.708 |
| NPFF1-Tango | −0.095 | −1.359 | 0.488 | −0.261 | −0.299 | −0.966 | −1.108 | 0.200 |
| NPFF2-Tango | −0.151 | 0.959 | 0.259 | 0.093 | 0.523 | 0.226 | 0.529 | −0.066 |
| NPS-Tango | 0.123 | 0.076 | −0.472 | −0.504 | −0.268 | −1.212 | −0.473 | −0.506 |
| NPY1R-Tango | 0.094 | 0.539 | −0.084 | 0.024 | 0.156 | −0.902 | −0.180 | −0.073 |
| NPY2R-Tango | 0.067 | 0.895 | 0.454 | −0.034 | 0.340 | −1.628 | 0.507 | −0.481 |
| NPY4R-Tango | 0.453 | 0.260 | 0.295 | −0.297 | −0.327 | −0.098 | −0.034 | −0.073 |
| NPY5R-Tango | −0.058 | 0.357 | 0.262 | 0.098 | 0.636 | 0.236 | 0.295 | −0.889 |
| NTSR1-Tango | 0.273 | 0.497 | −0.323 | 0.819 | 0.974 | 0.216 | 0.078 | −0.477 |
| NTSR2-Tango | −0.134 | 0.443 | −0.322 | 0.531 | 0.452 | 0.234 | −0.013 | −0.462 |
| OPN3-Tango | −0.068 | −0.207 | 0.280 | 0.465 | 0.146 | −0.266 | −0.266 | −0.155 |
| OPN5-Tango | 0.198 | 0.540 | 0.026 | 0.199 | 0.918 | 0.231 | 0.347 | −0.612 |
| OPRD1-Tango | −0.222 | 0.457 | 0.070 | −0.192 | −0.192 | 0.170 | −0.673 | −0.444 |
| OPRK1-Tango | −0.003 | 0.694 | −0.109 | −0.390 | 0.084 | 0.191 | −0.319 | −0.237 |
| OPRL1-Tango | −0.071 | −0.194 | −0.170 | −0.588 | −0.548 | −0.064 | −0.899 | 0.418 |
| OPRM1-Tango | −0.221 | 0.226 | 0.221 | −0.303 | 0.348 | 0.398 | −0.346 | −0.082 |
| OXER1-Tango | 0.334 | 0.336 | 0.008 | −0.107 | 0.547 | 0.163 | 0.122 | −0.237 |
| OXGR1-Tango | −0.011 | 0.297 | −0.018 | −0.038 | 0.103 | 0.345 | −0.069 | −0.217 |
| OXTR-Tango | −0.060 | 0.457 | 0.120 | 0.459 | 0.027 | 0.384 | −0.177 | −0.210 |
| P2RY1-Tango | −0.038 | 0.592 | −0.467 | −0.433 | −0.012 | 0.572 | 0.207 | −0.811 |
| P2RY10-Tango | −0.063 | 0.284 | −0.279 | −0.357 | −0.065 | 0.233 | −0.308 | −0.213 |
| P2RY11-Tango | −0.066 | 0.755 | −0.446 | −0.032 | 0.116 | 0.503 | 0.422 | −0.262 |
| P2RY12-Tango | −0.239 | 1.486 | 0.357 | 0.218 | 0.578 | 0.564 | 0.381 | 0.501 |
| P2RY13-Tango | −0.098 | 0.232 | 0.219 | −0.121 | 0.238 | 0.297 | 0.029 | −0.252 |
| P2RY14-Tango | 0.063 | −0.097 | 0.270 | −0.165 | 0.105 | −0.106 | −0.134 | −0.392 |
| P2RY2-Tango | −0.212 | 0.627 | 0.180 | −0.403 | −0.026 | 0.345 | −0.003 | −0.443 |
| P2RY4-Tango | 0.075 | 0.810 | 0.214 | −0.739 | −0.002 | 0.489 | 0.070 | −0.338 |
| P2RY6-Tango | 0.134 | 0.186 | −0.046 | −0.261 | −0.136 | −0.122 | −0.232 | −0.473 |
| P2RY8-Tango | 0.333 | 0.160 | −0.009 | −0.734 | −0.307 | −0.037 | −0.405 | −0.494 |
| PK1-Tango | −0.081 | 0.017 | −0.118 | −0.541 | −0.483 | −0.049 | −0.898 | −0.907 |
| PK2-Tango | 0.000 | 0.430 | 0.021 | 0.025 | 0.114 | 0.515 | 0.074 | 0.000 |
| PRRP-Tango | −0.238 | 0.436 | 0.187 | −0.044 | 0.487 | 0.365 | 0.118 | −0.452 |
| PTAFR-Tango | 0.071 | 0.410 | 0.097 | 0.248 | 0.423 | 0.528 | 0.373 | −0.914 |
| PTGDR-Tango | −0.346 | 0.646 | −0.311 | −0.196 | −0.380 | 0.248 | −0.305 | −0.620 |
| PTGER1-Tango | 0.415 | 0.639 | −0.122 | −0.092 | 0.000 | 0.188 | −0.216 | −0.165 |
| PTGER2-Tango | 0.506 | 0.707 | 0.058 | −0.071 | −0.489 | 0.748 | 0.034 | 0.126 |
| PTGER3-Tango | −0.409 | 0.675 | −0.211 | 0.016 | 0.380 | 0.675 | 0.385 | 0.118 |
| PTGER4-Tango | −0.422 | 0.902 | 0.005 | 0.326 | −0.129 | 0.439 | −0.027 | −0.422 |
| PTGFR-Tango | −0.087 | 0.449 | −0.716 | −0.180 | −0.386 | 0.819 | 0.220 | −0.628 |
| PTGIR-Tango | −0.463 | 0.733 | −0.076 | −0.382 | −0.640 | 0.913 | 0.699 | −0.611 |
| PTH1R-Tango | 0.280 | 0.512 | 0.035 | −0.363 | −0.147 | −0.166 | −0.354 | 0.054 |
| PTH2R-Tango | −0.182 | 0.406 | 0.313 | −0.316 | −0.308 | 0.357 | 0.252 | −0.852 |
| QRFP-Tango | −0.425 | 0.550 | −0.072 | −0.357 | −0.347 | 0.474 | −0.038 | −0.425 |
| RXFP1-Tango | −0.400 | 0.049 | −0.307 | −0.266 | −0.100 | −0.264 | −0.757 | −0.689 |
| RXFP2-Tango | −0.243 | 0.489 | 0.067 | −0.785 | −0.159 | 0.210 | 0.296 | −0.895 |
| RXFP3-Tango | −0.060 | 0.000 | 0.103 | −0.595 | −0.814 | −0.012 | −0.753 | −0.572 |
| RXFP4-Tango | 0.399 | 0.148 | −0.627 | 0.261 | 0.284 | 0.328 | 0.000 | −0.627 |
| S1PR1-Tango | 0.067 | 0.608 | 0.111 | 0.888 | 1.598 | 0.447 | 0.242 | −0.413 |
| S1PR2-Tango | −0.325 | 0.513 | −0.254 | 0.734 | 0.726 | 0.263 | 0.047 | −0.507 |
| S1PR3-Tango | 0.000 | 1.035 | 0.024 | 0.272 | −0.075 | 0.208 | 0.238 | −0.425 |
| S1PR4-Tango | 0.414 | 0.337 | −0.101 | 0.260 | 0.124 | 0.048 | −0.004 | −0.813 |
| S1PR5-Tango | −0.073 | 0.681 | −0.304 | 0.000 | 0.106 | 0.505 | 0.141 | −0.376 |
| SCTR-Tango | −0.239 | 0.749 | 0.086 | 0.435 | 0.154 | 0.260 | 0.086 | −0.358 |
| SSTR1-Tango | 0.485 | 0.592 | −0.341 | −0.482 | −1.130 | 0.672 | −0.541 | 0.127 |
| SSTR2-Tango | 0.062 | 0.239 | 0.050 | 0.445 | 0.333 | 0.405 | 0.221 | −0.503 |
| SSTR3-Tango | 0.115 | 0.670 | −0.080 | 0.000 | 0.556 | 0.564 | 0.564 | 0.047 |
| SSTR4-Tango | −0.092 | 0.322 | 0.428 | −0.045 | −0.100 | −0.038 | −0.399 | −1.531 |
| SSTR5-Tango | 0.126 | 0.775 | 1.253 | 0.475 | 0.108 | 0.820 | −0.287 | 0.541 |
| SUCNR1-Tango | −0.248 | 0.174 | 0.935 | 2.152 | −0.505 | 0.776 | −0.198 | 0.105 |
| TA1-Tango | −0.191 | 0.558 | 0.313 | −0.386 | 0.110 | 0.513 | −0.307 | −0.460 |
| TAAR2-Tango | −0.064 | 0.340 | −0.354 | −0.090 | −0.162 | 0.474 | −0.802 | −0.520 |
| TAAR5-Tango | −0.040 | −0.896 | −0.454 | −0.443 | −0.543 | −0.731 | −1.400 | −0.491 |
| TAAR6-Tango | 0.066 | −0.217 | 0.000 | −0.629 | −0.121 | −0.112 | −0.186 | −0.703 |
| TAAR8-Tango | −0.216 | −0.358 | −0.144 | −0.299 | −0.132 | −0.165 | −0.291 | −0.258 |
| TAAR9-Tango | 0.299 | −0.140 | −0.275 | −0.477 | −0.382 | −0.246 | −0.284 | −0.452 |
| TACR1-Tango | 0.015 | 0.052 | −0.030 | −0.116 | −0.021 | 0.041 | −0.004 | −0.601 |
| TACR2-Tango | −0.353 | 0.055 | −0.082 | −0.016 | −0.045 | 0.084 | −0.364 | −0.366 |
| TACR3-Tango | −0.147 | 0.589 | −0.034 | 0.028 | 0.257 | 0.388 | 0.258 | −0.675 |
| TBXA2R-Tango | −0.149 | −0.022 | 0.193 | −0.095 | −0.176 | −0.125 | −0.153 | −0.080 |
| TSHR-Tango | −0.071 | 0.121 | 0.102 | −0.849 | −0.413 | −0.257 | 0.061 | −0.753 |
| UTS2R-Tango | −0.052 | 0.327 | 0.318 | 0.037 | −0.266 | −0.142 | −0.160 | −0.143 |
| VIPR1-Tango | −0.696 | −0.786 | 0.042 | −0.200 | −0.200 | −0.975 | −1.097 | −0.759 |
| VIPR2-Tango | −0.896 | 0.340 | 1.100 | 0.000 | 0.000 | 0.303 | 0.758 | −0.615 |

| PRESTO-Tango | Bifidobacterium | Bifidobacterium | Bifidobacterium | Morganella | Morganella | Morganella | Morganella | Morganella |
|---|---|---|---|---|---|---|---|---|
| with GMM | adolescentis-1# | adolescentis-2# | adolescentis-3# | spp-1# | spp-2# | spp-3# | spp-4# | spp-5# |
| ADCYAP1R1- | −0.021 | −0.014 | −0.160 | −0.862 | −0.387 | −0.531 | 0.025 | −0.248 |
| Tango | | | | | | | | |
| ADORA1-Tango | −0.262 | −0.045 | −0.393 | −1.367 | −0.177 | −0.366 | −0.237 | −0.359 |
| ADORA2A-Tango | −0.178 | −0.430 | −0.270 | −1.277 | 0.021 | −0.017 | 0.209 | −0.540 |
| ADORA2B-Tango | −0.146 | −0.342 | −0.152 | −0.770 | −0.376 | −0.279 | −0.089 | −0.260 |
| ADORA3-Tango | 0.333 | 0.168 | 0.251 | −0.347 | −0.369 | −0.549 | −0.369 | −0.416 |
| ADRA1A-Tango | −0.216 | −0.203 | −0.114 | −0.623 | −0.235 | −0.285 | 0.012 | 0.098 |
| ADRA1B-Tango | −0.416 | −0.327 | 0.294 | −1.219 | −0.538 | −0.672 | −0.891 | −0.808 |
| ADRA1D-Tango | −0.748 | −0.150 | −0.138 | −0.886 | −0.229 | −0.434 | 0.174 | 0.017 |
| ADRA2A-Tango | 0.073 | 0.136 | −0.106 | 0.125 | 0.793 | 0.816 | 1.246 | 1.216 |
| ADRA2B-Tango | 0.078 | −0.055 | −0.515 | −1.660 | 0.252 | 0.662 | 0.258 | 0.679 |
| ADRA2C-Tango | 0.065 | −0.531 | −0.176 | −1.431 | 0.000 | 1.482 | 1.186 | 1.042 |
| ADRB1-Tango | 0.028 | −0.077 | 0.138 | −0.266 | 0.553 | 0.669 | 0.230 | 0.429 |
| ADRB2-Tango | −0.057 | −0.308 | −0.564 | −2.203 | −0.799 | −0.285 | 0.133 | −0.528 |
| ADRB3-Tango | −0.235 | −0.411 | −0.292 | −1.644 | −0.595 | 0.125 | −0.471 | 0.106 |
| AGTR1-Tango | −0.164 | −0.259 | −0.147 | −1.490 | −0.262 | 0.015 | 0.382 | 0.593 |
| AGTR2-Tango | −0.104 | −0.091 | 0.288 | −0.508 | 0.246 | −0.242 | 0.442 | 0.442 |
| APJ-Tango | 0.312 | 0.010 | 0.096 | −1.393 | −0.255 | −0.442 | −0.318 | 0.208 |
| AVPR1A-Tango | 0.547 | 0.296 | 0.280 | −2.619 | −0.642 | −0.514 | −0.520 | −0.595 |
| AVPR1B-Tango | −0.160 | −0.150 | −0.098 | −1.362 | −0.328 | −0.365 | −0.188 | −0.427 |
| AVPR2-Tango | −0.227 | −0.222 | −0.247 | −1.982 | −0.232 | −0.221 | −0.347 | −0.270 |
| BB3-Tango | −0.302 | −0.231 | −0.276 | −0.806 | −0.469 | 0.071 | −0.520 | −0.476 |
| BDKBR1-Tango | −0.409 | −0.198 | −0.116 | −1.122 | 0.176 | −0.018 | −0.341 | −0.026 |
| BDKBR2-Tango | −0.230 | −0.267 | 0.247 | −1.050 | −0.061 | −0.197 | 0.021 | 0.191 |
| C3AR1-Tango | −0.416 | −0.451 | −0.067 | −0.723 | −0.314 | 0.083 | 0.219 | −0.054 |
| C5A-Tango | 0.106 | 0.024 | 0.175 | −1.125 | −0.273 | −0.262 | −0.591 | −0.647 |
| CALCRb-Tango | −0.073 | −0.139 | −0.043 | −1.348 | −0.191 | −0.023 | −0.698 | −0.183 |
| CALCRL-Tango | −0.015 | −0.135 | −0.117 | −1.096 | −0.143 | −0.012 | −0.242 | −0.224 |
| CASR-Tango | 0.221 | 0.112 | −0.379 | −1.494 | −0.905 | −0.503 | −1.313 | −1.405 |
| CCKAR-Tango | 0.037 | −0.030 | 0.294 | −1.243 | −0.401 | −0.449 | −0.503 | −0.534 |
| CCKBR-Tango | −0.089 | −0.179 | 0.003 | −1.218 | −0.009 | −0.106 | −0.559 | −0.650 |
| CCR10-Tango | −0.264 | −0.578 | −0.402 | −1.876 | −0.394 | −0.580 | −0.549 | −0.564 |
| CCR2-Tango | −0.038 | −0.038 | 0.106 | −1.648 | −0.373 | 0.147 | −0.853 | −0.817 |
| CCR3-Tango | −0.155 | 0.100 | 0.225 | −2.027 | 0.139 | −0.065 | 0.213 | 0.445 |
| CCR4-Tango | 0.022 | −0.008 | 0.090 | −1.684 | −1.247 | −0.153 | −0.815 | −0.318 |
| CCR5-Tango | 0.149 | −0.372 | −0.036 | −1.419 | 0.107 | −0.566 | 0.308 | −0.454 |
| CCR6-Tango | −1.048 | −0.567 | 0.345 | −1.169 | 0.097 | 0.064 | 0.540 | 0.666 |
| CCR7-Tango | −0.288 | −0.020 | −0.681 | −1.422 | −0.532 | −0.471 | 0.878 | −0.565 |
| CCR8-Tango | 0.258 | −0.289 | −0.487 | −2.045 | −0.412 | −0.435 | 0.080 | 0.466 |
| CCRL2-Tango | −0.289 | −0.021 | −0.297 | −1.122 | −0.339 | −0.045 | −0.077 | 0.415 |
| CD97-Tango | −0.705 | −0.705 | −0.302 | −0.177 | 0.262 | 0.327 | −0.621 | −0.451 |
| CHRM1-Tango | 0.000 | −0.102 | 0.266 | −0.384 | −0.069 | −0.081 | 0.463 | 0.355 |
| CHRM2-Tango | 0.117 | 0.022 | −0.492 | −0.717 | −0.495 | −0.147 | −0.101 | −0.207 |
| CHRM3-Tango | 0.019 | −0.148 | −0.130 | −0.372 | −0.012 | 0.058 | 0.628 | 0.521 |
| CHRM4-Tango | 0.078 | −0.055 | 0.135 | −1.099 | 0.059 | −0.310 | 0.229 | 0.265 |
| CHRM5-Tango | −0.014 | −0.091 | −0.298 | −1.106 | −0.389 | −0.162 | −0.024 | −0.312 |
| CMKLR1-Tango | −0.046 | −0.330 | −0.032 | −1.368 | −0.432 | −0.386 | −0.170 | −0.342 |
| CNR1-Tango | −0.021 | −0.030 | −0.172 | −1.180 | −0.589 | −0.478 | −0.011 | −0.241 |
| CNR2-Tango | −0.537 | −0.580 | −0.046 | −1.461 | −0.272 | −0.381 | −0.077 | −0.058 |
| CRHR1-Tango | −0.170 | 0.000 | 0.261 | −1.396 | −0.399 | −0.730 | 0.796 | −0.341 |
| CRHR2-Tango | 0.433 | 0.239 | 0.417 | −1.546 | −0.298 | −0.919 | −0.095 | 0.533 |
| CX3C1-Tango | 0.050 | −0.477 | −0.069 | −1.420 | 0.358 | −0.643 | −0.034 | 0.180 |
| CXCR1-Tango | 0.302 | −0.033 | 0.149 | −1.902 | 0.069 | 0.023 | 0.942 | 0.336 |
| CXCR2-Tango | −0.336 | −0.129 | −0.212 | −0.503 | 0.530 | 0.828 | 1.120 | 1.024 |
| CXCR3-Tango | 0.010 | 0.165 | −0.330 | −1.438 | −0.139 | −0.152 | 0.326 | 0.252 |
| CXCR4-Tango | 0.036 | −0.203 | 0.031 | −1.075 | −0.141 | −0.117 | 0.371 | 0.357 |
| CXCR5-Tango | −0.263 | −0.511 | 0.037 | −0.475 | −0.297 | 0.294 | 0.365 | 0.123 |
| CXCR6-Tango | 0.155 | −0.229 | −0.147 | −1.505 | 0.087 | −0.133 | 1.000 | 1.129 |
| CXCR7-Tango | −0.154 | 0.130 | −0.428 | −1.316 | −0.271 | −0.076 | 0.609 | 0.456 |
| CYSLTR1-Tango | 0.321 | 0.068 | 0.180 | −1.455 | 0.209 | 0.475 | 0.646 | 0.599 |
| CYSLTR2-Tango | 0.224 | 0.540 | −0.058 | −0.852 | 0.062 | 0.510 | 0.615 | 0.500 |
| DRD1-Tango | 0.143 | −0.050 | −0.442 | −2.319 | −0.680 | 0.875 | 1.191 | 1.025 |
| DRD2-Tango | 0.025 | −0.259 | −0.659 | 2.541 | 3.090 | 3.791 | 4.037 | 4.196 |
| DRD3-Tango | −0.655 | 0.098 | −1.091 | 1.890 | 1.730 | 3.375 | 3.895 | 3.843 |
| DRD4-Tango | −1.087 | −0.052 | −1.000 | 2.421 | 1.149 | 2.979 | 2.966 | 3.231 |
| DRD5-Tango | −0.015 | 0.015 | −0.367 | −1.975 | −0.386 | −0.556 | −0.029 | 0.000 |
| EDNRA-Tango | 0.292 | 0.220 | 0.182 | −2.107 | −0.583 | −0.548 | −0.176 | 0.081 |
| EDNRB-Tango | 0.069 | −0.012 | −0.316 | −1.840 | −0.342 | −0.064 | −0.326 | −0.353 |
| ELTD1-Tango | −0.022 | −0.013 | −0.271 | −2.942 | −0.422 | 0.041 | −0.481 | −0.587 |
| F2R-Tango | −0.038 | −0.046 | 0.294 | −3.510 | −0.442 | −0.339 | −0.356 | −0.165 |
| F2RL1-Tango | 0.042 | −0.020 | 0.098 | −3.035 | −0.586 | −0.493 | −0.227 | −0.073 |
| F2RL2-Tango | −0.059 | −0.030 | −0.067 | −3.104 | −0.864 | −0.402 | −0.426 | −0.547 |
| F2RL3-Tango | 0.038 | −0.044 | 0.064 | −2.733 | −0.556 | −0.603 | −0.114 | −0.090 |
| FFA1-Tango | 0.343 | 0.318 | −0.224 | −1.180 | −0.173 | −0.094 | −0.223 | −0.158 |
| FFA2-Tango | 0.549 | 0.566 | −0.271 | −1.681 | −0.498 | 0.121 | −0.079 | −0.065 |
| FFA3-Tango | 0.239 | 0.322 | −0.056 | −1.266 | −0.581 | −0.050 | 0.256 | 0.245 |
| FPR1-Tango | 0.543 | 0.420 | −0.033 | −0.471 | 0.495 | 0.841 | 1.034 | 1.068 |
| FPR2-Tango | 0.131 | 0.106 | −0.239 | −1.179 | −0.212 | 0.202 | 0.458 | 0.386 |
| FPR3-Tango | 0.386 | 0.357 | −0.121 | −1.524 | −0.265 | −0.388 | 0.006 | 0.136 |
| FSHR-Tango | 0.268 | 0.370 | −0.132 | −1.948 | −0.858 | −0.435 | −0.495 | −0.437 |
| GABBR1-Tango | 0.098 | 0.154 | −0.111 | −0.149 | 0.581 | 0.516 | 0.589 | 0.576 |
| GAL1-Tango | −0.411 | −0.553 | −0.395 | −2.499 | −0.219 | −0.799 | −0.501 | −0.383 |
| GAL2-Tango | 0.166 | 0.496 | 0.362 | −2.824 | −0.671 | −1.016 | 0.885 | 0.885 |
| GAL3-Tango | −0.300 | −0.300 | 0.322 | −1.617 | −0.650 | −1.198 | −1.040 | −0.772 |
| GCGR-Tango | 0.372 | 0.154 | −0.140 | −1.878 | −0.777 | −0.534 | −0.258 | −0.301 |
| GHRHR-Tango | −0.042 | −0.244 | 0.228 | −1.560 | −0.195 | −0.635 | −0.606 | −0.575 |
| GHSR-Tango | 0.043 | −0.144 | −0.127 | −2.110 | −0.842 | −0.614 | −0.086 | 0.270 |
| GIPR-Tango | −0.132 | −0.341 | 0.043 | −2.087 | −0.476 | −0.893 | −0.335 | −0.040 |
| GLP1R-Tango | 0.039 | −0.086 | 0.369 | −2.126 | −0.519 | −0.960 | −0.743 | −0.330 |
| GLP2R-Tango | 0.407 | 0.091 | −0.279 | −1.840 | −0.059 | −0.647 | −0.075 | 0.414 |
| GNRHR-Tango | −0.054 | −0.667 | −0.307 | −1.217 | −0.627 | −0.333 | 0.200 | 0.250 |
| GPBA-Tango | −0.395 | −0.330 | 0.140 | −0.924 | 0.260 | −0.391 | 0.645 | 1.075 |
| GPER-Tango | −0.267 | −0.246 | 0.246 | −1.463 | −0.339 | 0.040 | 0.000 | 0.330 |
| GPR1-Tango | 0.256 | 0.033 | −0.317 | −1.195 | −0.459 | −0.579 | −0.341 | −0.311 |
| GPR101-Tango | −0.188 | 0.074 | −0.117 | −0.749 | −0.077 | −0.494 | 0.069 | −0.072 |
| GPR110-Tango | −0.410 | −0.473 | 0.019 | −1.462 | −0.566 | −0.488 | −0.480 | −0.480 |
| GPR111-Tango | −0.643 | −0.511 | −0.432 | −1.176 | −0.493 | −0.126 | −0.225 | 0.258 |
| GPR113-Tango | −0.255 | 0.219 | −0.510 | −1.285 | −0.820 | −0.609 | −0.441 | −0.185 |
| GPR114-Tango | 0.239 | 0.261 | −0.172 | −0.852 | −0.135 | −0.443 | −0.368 | −0.053 |
| GPR115-Tango | 0.095 | 0.142 | −0.398 | −1.433 | −0.603 | −0.532 | −0.598 | −0.553 |
| GPR116-Tango | −0.232 | −0.255 | −0.326 | −1.131 | −0.315 | −0.256 | −0.509 | −0.358 |
| GPR119-Tango | −0.082 | −0.035 | −0.230 | −1.510 | −0.268 | −0.126 | −0.018 | 0.141 |
| GPR12-Tango | −0.537 | −0.363 | −0.132 | −1.745 | 0.266 | 0.015 | 0.194 | 0.236 |
| GPR120-Tango | −0.347 | −0.490 | −0.190 | −0.754 | 0.126 | −0.164 | −0.198 | 0.054 |
| GPR123-Tango | 0.339 | 0.345 | 0.151 | −1.147 | −0.494 | −0.600 | 0.131 | 0.188 |
| GPR124-Tango | −0.503 | −0.612 | −0.748 | −1.302 | −0.748 | −0.259 | −0.640 | −0.578 |
| GPR125-Tango | −0.334 | −0.383 | −0.980 | −1.027 | −0.687 | −0.711 | −0.140 | −0.289 |
| GPR126-Tango | −0.323 | −0.366 | −0.857 | −1.516 | −0.145 | −0.359 | −0.307 | −0.437 |
| GPR132-Tango | −0.066 | −0.011 | −0.643 | −1.841 | −0.985 | −0.844 | −0.736 | −0.762 |
| GPR133-Tango | 0.004 | 0.031 | −0.826 | −1.622 | −0.947 | −0.546 | −0.864 | −0.683 |
| GPR135-Tango | −0.041 | −0.297 | −0.316 | −1.647 | −0.350 | −0.440 | −0.267 | −0.267 |
| GPR141-Tango | −0.925 | −0.937 | −0.598 | −0.738 | 0.023 | 0.525 | 0.411 | 0.203 |
| GPR142-Tango | −0.678 | −0.696 | −0.722 | −1.612 | −0.602 | −0.828 | −0.133 | 0.017 |
| GPR143-Tango | 0.396 | 0.141 | 0.074 | −2.535 | −0.769 | −0.704 | −0.368 | −0.461 |
| GPR144-Tango | 0.024 | −0.242 | −0.449 | −2.271 | −0.803 | −0.174 | −0.523 | −0.840 |
| GPR146-Tango | −0.241 | −0.485 | 0.117 | −1.780 | 0.358 | 0.812 | −0.217 | 0.481 |
| GPR148-Tango | −0.649 | −0.904 | 0.341 | −1.387 | −0.404 | −0.170 | 0.147 | 1.496 |
| GPR149-Tango | 0.454 | 0.106 | −1.300 | −2.534 | −0.522 | −0.160 | 0.523 | 0.208 |
| GPR15-Tango | 0.040 | 0.435 | −0.305 | −2.273 | −0.326 | 0.177 | 0.133 | 0.403 |
| GPR150-Tango | −0.542 | −0.420 | −0.054 | −1.063 | 0.052 | 0.144 | 0.282 | 1.200 |
| GPR151-Tango | 0.388 | 0.117 | −0.663 | −1.873 | −0.198 | −0.471 | −0.302 | −0.559 |
| GPR152-Tango | 0.060 | 0.034 | 0.010 | −0.983 | −0.480 | −0.664 | −0.591 | −0.644 |
| GPR153-Tango | 0.173 | 0.233 | 0.244 | −1.114 | −0.601 | −0.404 | −0.173 | −0.051 |
| GPR156-Tango | −0.082 | −0.027 | −0.189 | −0.180 | 0.026 | −0.706 | 0.082 | 0.250 |
| GPR157-Tango | −0.295 | −0.231 | −0.148 | −0.916 | −0.387 | −0.143 | −0.478 | 0.028 |
| GPR158-Tango | −0.050 | −0.068 | 0.058 | −1.272 | −0.100 | −0.394 | −0.570 | −0.440 |
| GPR160-Tango | 0.238 | 0.327 | −0.362 | −0.936 | −0.683 | −0.194 | 0.000 | 0.160 |
| GPR161-Tango | −0.106 | −0.123 | −0.518 | −1.177 | −0.195 | −0.030 | 0.246 | 0.550 |
| GPR162-Tango | −0.400 | −0.230 | −0.701 | −1.383 | −0.608 | −0.299 | −0.287 | −0.424 |
| GPR17-Tango | 0.684 | 0.693 | −0.658 | −0.358 | −0.288 | 0.048 | 0.544 | 0.415 |
| GPR171-Tango | 0.355 | 0.212 | −0.475 | −0.263 | −0.378 | 0.265 | 0.013 | 0.159 |
| GPR173-Tango | −0.420 | −0.157 | −0.441 | −1.135 | −0.298 | −0.047 | 0.000 | 0.076 |
| GPR174-Tango | −0.120 | −0.497 | −0.187 | −1.503 | −0.088 | 0.164 | 0.365 | 0.239 |
| GPR18-Tango | −0.192 | −0.477 | −0.581 | −1.757 | −0.508 | −0.344 | −0.112 | 0.320 |
| GPR182-Tango | 0.179 | 0.123 | −0.542 | −1.088 | −0.652 | −0.080 | −0.126 | −0.005 |
| GPR183-Tango | −0.027 | −0.180 | −0.541 | −1.381 | −0.336 | −0.205 | −0.051 | −0.295 |
| GPR19-Tango | 0.170 | 0.121 | −0.518 | −0.851 | −0.847 | −0.371 | −0.580 | −0.480 |
| GPR20-Tango | −0.361 | −0.312 | −0.753 | −1.303 | 0.047 | 0.000 | 0.511 | −0.621 |
| GPR21-Tango | −0.110 | 0.124 | −0.146 | −1.314 | 0.064 | 0.150 | 0.075 | 0.340 |
| GPR22-Tango | −0.118 | −0.489 | −0.234 | −1.339 | −0.038 | −0.270 | 0.405 | 0.547 |
| GPR25-Tango | −0.415 | −0.016 | −0.131 | −1.540 | 0.031 | −0.190 | −0.220 | 0.371 |
| GPR26-Tango | −0.241 | −0.696 | −0.402 | −1.851 | −0.054 | −0.288 | 0.093 | −0.374 |
| GPR27-Tango | 0.166 | −0.118 | 0.221 | −0.857 | 0.330 | 0.310 | 0.880 | 0.829 |
| GPR3-Tango | 0.223 | 0.156 | −0.247 | −1.470 | −0.498 | 0.079 | 0.478 | 0.423 |
| GPR31-Tango | −0.040 | −0.090 | −0.419 | −1.670 | −0.465 | 0.192 | 0.339 | 0.154 |
| GPR32-Tango | 0.266 | 0.290 | −0.353 | −1.689 | 0.000 | −0.710 | 0.064 | 0.100 |
| GPR34-Tango | 0.239 | −0.143 | −0.993 | −1.366 | −0.385 | −0.145 | 0.125 | 0.300 |
| GPR35-Tango | 0.047 | 0.127 | −0.298 | −2.127 | −0.502 | −0.614 | −0.152 | −0.085 |
| GPR37-Tango | −0.108 | −0.026 | −0.155 | −1.302 | −0.147 | −0.401 | 0.143 | 0.242 |
| GPR37L1-Tango | −0.372 | −0.379 | −0.330 | −1.620 | −0.188 | −0.631 | −0.367 | −0.374 |
| GPR39-Tango | −0.399 | −0.309 | −0.259 | −1.698 | −0.212 | −0.546 | −0.636 | −0.263 |
| GPR4-Tango | −0.136 | −0.222 | −0.144 | −1.464 | −0.061 | −0.349 | 0.123 | 0.236 |
| GPR44-Tango | −0.410 | −0.283 | −0.534 | −0.997 | −0.202 | −0.298 | 0.194 | −0.141 |
| GPR45-Tango | 0.293 | −0.087 | −0.515 | −2.048 | −0.551 | −0.144 | −0.455 | −0.737 |
| GPR50-Tango | 0.252 | 0.214 | 0.477 | −1.364 | −0.264 | 0.061 | 0.720 | 0.593 |
| GPR52-Tango | −0.401 | −0.222 | −0.215 | −0.695 | 0.812 | −0.102 | 0.805 | 0.565 |
| GPR55-Tango | 0.084 | 0.337 | 0.012 | −1.227 | −0.359 | −0.215 | 0.551 | 0.372 |
| GPR56-Tango | −0.271 | −0.387 | −0.016 | −1.318 | −0.379 | 0.114 | 0.173 | 0.217 |
| GPR6-Tango | 0.063 | 0.221 | −0.286 | −1.572 | 0.284 | 0.242 | 0.236 | 0.010 |
| GPR61-Tango | 0.373 | 0.334 | −0.038 | −1.380 | −0.146 | −0.598 | 0.020 | −0.018 |
| GPR62-Tango | 0.096 | 0.119 | −0.264 | −0.615 | −0.248 | 0.245 | −0.269 | −0.790 |
| GPR63-Tango | 0.010 | −0.225 | −0.166 | −0.862 | −0.554 | −0.769 | −0.437 | −0.351 |
| GPR64-Tango | −0.022 | −0.187 | −0.338 | −1.198 | −0.384 | −0.939 | −0.009 | −0.349 |
| GPR65-Tango | 0.414 | 0.453 | 0.032 | −1.372 | 0.015 | −0.473 | −0.129 | −0.332 |
| GPR68-Tango | 0.211 | −0.111 | −0.217 | −1.639 | −0.442 | −0.760 | −0.163 | −0.649 |
| GPR75-Tango | 0.137 | 0.258 | 0.139 | −1.441 | 0.115 | −0.436 | 0.229 | 0.000 |
| GPR77-Tango | 0.080 | 0.035 | −0.184 | −1.661 | −0.318 | −0.529 | −0.525 | −0.468 |
| GPR78-Tango | 0.272 | 0.077 | −0.159 | −1.695 | 0.053 | 0.043 | −0.096 | −0.571 |
| GPR82-Tango | −0.048 | −0.164 | −0.362 | −2.910 | −0.478 | −0.410 | −0.082 | −0.311 |
| GPR83-Tango | 0.148 | −0.056 | 0.130 | −1.472 | −0.044 | −0.680 | −0.176 | −0.234 |
| GPR84-Tango | 0.056 | 0.051 | 0.079 | −1.156 | −0.066 | −0.351 | −0.220 | −0.340 |
| GPR85-Tango | −0.121 | −0.130 | 0.023 | −0.929 | 0.133 | 0.059 | 0.023 | 0.051 |
| GPR87-Tango | −0.139 | 0.036 | 0.304 | −0.620 | 0.129 | −0.034 | 0.246 | 0.588 |
| GPR88-Tango | 0.030 | −0.168 | −0.165 | −0.804 | 0.277 | −0.297 | 0.121 | 0.346 |
| GPR97-Tango | 0.182 | 0.228 | −0.032 | −1.045 | −0.018 | −0.203 | −0.030 | 0.036 |
| GPRC5A-Tango | 0.069 | 0.114 | 0.142 | −1.396 | 0.041 | −0.337 | −0.391 | −0.144 |
| GPRC5B-Tango | 0.190 | −0.010 | 0.002 | −1.344 | −0.235 | −0.109 | −0.150 | −0.487 |
| GPRC5C-Tango | 0.508 | 0.258 | 0.693 | −0.732 | 0.497 | −0.343 | −0.002 | 0.098 |
| GPRC5D-Tango | −0.515 | −0.448 | 0.320 | −1.124 | −0.122 | −0.946 | −0.230 | −0.230 |
| GPRC6A-Tango | −0.431 | −0.557 | 0.469 | −1.150 | −0.016 | −0.827 | −0.347 | −0.481 |
| GRM1-Tango | −0.559 | −0.628 | 0.049 | −1.376 | −0.162 | −0.512 | −0.327 | −0.052 |
| GRM2-Tango | −0.541 | −0.449 | 0.062 | −1.132 | 0.050 | −0.282 | −0.073 | −0.236 |
| GRM4-Tango | 0.293 | 0.267 | 0.579 | −1.356 | −0.108 | −0.163 | −0.319 | −0.314 |
| GRM5-Tango | −0.325 | −0.354 | 0.229 | −1.146 | −0.362 | −0.923 | −0.347 | −0.309 |
| GRM6-Tango | −0.597 | −0.607 | −0.514 | −0.957 | −0.250 | −0.717 | 0.089 | 0.039 |
| GRM7-Tango | −0.804 | −0.749 | 0.316 | −1.681 | −0.067 | −0.980 | −0.434 | −0.198 |
| GRM8-Tango | −0.534 | −0.913 | 0.042 | −0.956 | −0.261 | −0.434 | 0.158 | 0.267 |
| GRPR-Tango | −0.023 | −0.531 | 0.485 | −1.659 | −0.097 | −0.446 | 0.420 | −0.219 |
| HCA1-Tango | 0.028 | −0.399 | −0.149 | −1.726 | −0.023 | −0.019 | −0.138 | −0.108 |
| HCA2-Tango | 0.366 | 0.190 | 0.806 | −1.168 | 0.495 | −0.332 | 0.574 | 0.930 |
| HCA3-Tango | −0.056 | −0.212 | 0.000 | −1.831 | −0.440 | 0.037 | 0.097 | 0.158 |
| HCTR1-Tango | −0.156 | −0.310 | −0.376 | −0.745 | −0.036 | 0.760 | 0.248 | 0.682 |
| HCTR2-Tango | 0.267 | 0.152 | −0.094 | −0.953 | −0.471 | −0.078 | 0.122 | 0.234 |
| HRH1-Tango | −0.033 | 0.277 | 0.092 | −1.485 | 0.311 | −0.449 | 0.770 | 0.552 |
| HRH2-Tango | 0.026 | 0.197 | 0.274 | −0.316 | 0.045 | 0.230 | 0.669 | 0.607 |
| HRH3-Tango | −0.381 | −0.213 | 0.050 | 1.247 | 1.510 | 0.805 | 2.290 | 2.201 |
| HRH4-Tango | −0.670 | −0.582 | −0.656 | 0.447 | 1.187 | 1.163 | 1.909 | 2.005 |
| HTR1A-Tango | 0.298 | −0.042 | 0.096 | −1.166 | 0.581 | 0.797 | 0.906 | 0.711 |
| HTR1B-Tango | −0.011 | 0.029 | 0.255 | −1.618 | 0.120 | −0.129 | 0.767 | 0.762 |
| HTR1D-Tango | −0.206 | −0.041 | −0.164 | −1.400 | 0.061 | 0.110 | 0.705 | 0.472 |
| HTR1E-Tango | −0.022 | −0.003 | −0.176 | −1.088 | −0.132 | −0.385 | 0.342 | 0.278 |
| HTR1F-Tango | 0.357 | 0.203 | 0.536 | −1.610 | −0.482 | −0.345 | 0.685 | 0.465 |
| HTR2A-Tango | 0.138 | 0.100 | 0.037 | −1.489 | −0.316 | −0.169 | 0.063 | 0.188 |
| HTR2B-Tango | 0.085 | 0.105 | −1.322 | −1.148 | −0.148 | −0.234 | 0.263 | 0.287 |
| HTR2C-Tango | 0.140 | −0.072 | −1.476 | −1.607 | −0.042 | −0.430 | −0.240 | −0.176 |
| HTR4-Tango | 0.125 | 0.089 | −1.597 | −1.676 | −0.156 | 0.195 | 0.181 | 0.648 |
| HTR5-Tango | 0.318 | 0.157 | −1.249 | −1.972 | −0.053 | −0.021 | 0.670 | 0.386 |
| HTR6-Tango | −0.380 | −0.195 | −1.114 | −1.252 | 0.264 | 0.508 | −0.047 | −0.047 |
| HTR7-Tango | 0.093 | −0.078 | −2.023 | −0.548 | 0.060 | −0.098 | 0.268 | 0.424 |
| KISSPEPTIN-R- | −0.031 | −0.330 | −0.092 | −1.962 | −0.308 | −0.127 | −1.000 | −0.333 |
| Tango | | | | | | | | |
| LHCGR-Tango | −0.541 | −0.410 | 0.562 | −2.806 | −0.426 | −0.472 | −0.486 | −0.540 |
| LPAR1-Tango | 0.191 | 0.256 | 0.591 | −3.406 | −0.037 | 0.046 | 0.165 | 0.036 |
| LPAR2-Tango | 0.006 | −0.141 | 0.246 | −2.775 | −0.434 | −0.459 | −0.503 | −0.481 |
| LPAR4-Tango | 0.064 | 0.047 | 0.344 | −2.953 | −0.022 | −0.280 | −0.334 | −0.521 |
| LPAR5-Tango | −0.046 | −0.039 | −0.078 | −2.420 | 0.007 | 0.065 | −0.142 | −0.012 |
| LPAR6-Tango | 0.106 | 0.057 | 0.165 | −1.407 | −0.166 | −0.164 | 0.500 | 0.553 |
| LTB4R-Tango | −0.022 | −0.194 | −0.468 | −1.629 | 0.000 | −0.212 | 0.338 | 0.455 |
| LTB4R2B-Tango | 0.118 | 0.316 | 0.486 | −1.530 | 0.022 | −0.457 | 0.086 | −0.153 |
| MAS1-Tango | −0.025 | 0.198 | 0.331 | −1.505 | 0.017 | 0.189 | −0.051 | 0.000 |
| MAS1L-Tango | 0.069 | 0.059 | 0.432 | −1.646 | −0.224 | −0.806 | −0.588 | −0.461 |
| MC1R-Tango | 0.082 | 0.173 | 0.160 | −1.223 | 0.047 | −0.323 | −0.198 | −0.384 |
| MC2R-Tango | 0.160 | 0.435 | 0.374 | −1.605 | −0.214 | −0.089 | 0.894 | 0.458 |
| MC3R-Tango | −0.115 | 0.160 | 0.391 | −1.439 | −0.117 | −0.284 | −0.122 | 0.112 |
| MC4R-Tango | 0.229 | −0.069 | 0.110 | −0.840 | 0.532 | 0.225 | 0.503 | 0.503 |
| MC5R-Tango | −0.333 | 0.135 | 0.229 | −2.578 | 0.754 | −0.260 | 0.768 | 0.514 |
| MCHR1-Tango | −0.372 | −0.672 | −0.850 | −2.015 | −1.113 | −1.061 | −0.298 | −0.269 |
| MCHR2-Tango | −0.037 | −0.395 | −0.568 | −0.913 | −0.568 | −0.780 | −0.071 | −0.098 |
| MLNR-Tango | 0.012 | −0.170 | −0.126 | −1.172 | 0.030 | −0.364 | 0.177 | 0.117 |
| MRGPRD-Tango | 0.088 | 0.056 | −0.171 | −1.924 | −0.517 | −0.402 | 0.100 | 0.229 |
| MRGPRE-Tango | 0.034 | 0.110 | −0.442 | −1.053 | −0.024 | −0.429 | 0.216 | 0.090 |
| MRGPRF-Tango | 0.148 | 0.003 | 0.711 | −0.687 | 0.423 | −0.258 | 0.323 | 0.433 |
| MRGPRG-Tango | −0.340 | −0.358 | 0.018 | −0.678 | −0.552 | −0.667 | −0.177 | 0.050 |
| MRGPRX1-Tango | −0.922 | −0.703 | −0.739 | −1.386 | −0.879 | −0.693 | 0.454 | 0.261 |
| MRGPRX2-Tango | 0.126 | 0.083 | −0.357 | −0.935 | −0.189 | −0.588 | 0.371 | 0.450 |
| MRGPRX3-Tango | −0.463 | −0.672 | −0.157 | −1.316 | −0.204 | −0.457 | 0.155 | −0.029 |
| MRGPRX4-Tango | −0.062 | −0.079 | −0.231 | −2.759 | −0.569 | −1.255 | −0.289 | −0.474 |
| MTNR1A-Tango | −0.227 | 0.084 | 0.389 | −2.351 | 0.415 | −0.547 | 0.255 | −0.771 |
| MTNR1B-Tango | 0.218 | 0.376 | 0.216 | −1.344 | 0.530 | −0.330 | 0.875 | 0.643 |
| NMBR-Tango | 0.831 | 0.742 | 0.695 | −1.362 | −0.123 | −0.876 | 0.105 | 0.022 |
| NMUR1-Tango | −0.626 | −0.140 | −0.071 | −1.591 | −0.361 | −0.179 | 0.042 | −0.766 |
| NMUR2-Tango | 0.329 | 0.236 | 0.357 | −2.693 | −0.313 | −0.596 | −0.272 | −0.480 |
| NPBW1-Tango | −0.485 | −0.298 | −0.798 | −0.613 | −0.077 | 0.340 | −0.047 | 0.350 |
| NPBW2-Tango | 0.148 | 0.201 | −0.880 | −0.932 | −0.230 | 0.579 | 0.413 | 0.196 |
| NPFF1-Tango | 0.221 | 0.265 | −0.124 | −0.850 | 0.138 | −1.408 | 0.214 | −0.411 |
| NPFF2-Tango | 0.543 | 0.407 | 0.337 | −2.348 | −0.125 | −0.755 | −0.392 | −0.183 |
| NPS-Tango | −0.239 | −0.361 | −0.865 | −1.779 | −0.478 | −1.363 | −0.407 | −1.153 |
| NPY1R-Tango | 0.285 | 0.389 | −0.528 | −2.304 | −0.077 | −1.066 | −0.499 | −0.642 |
| NPY2R-Tango | −0.782 | 0.571 | −0.358 | −0.913 | −0.183 | −0.754 | −0.365 | −0.628 |
| NPY4R-Tango | −0.710 | −0.523 | −0.589 | −1.260 | −0.260 | −0.671 | −0.065 | 0.000 |
| NPY5R-Tango | 0.636 | 0.591 | −0.117 | −0.789 | 0.132 | −0.123 | 0.096 | −0.035 |
| NTSR1-Tango | 1.125 | 0.938 | −0.011 | −2.297 | −0.111 | −0.250 | −0.029 | 0.019 |
| NTSR2-Tango | 0.572 | 0.849 | 0.354 | −2.188 | −0.111 | 0.112 | −0.565 | −0.635 |
| OPN3-Tango | 0.772 | 0.873 | 0.132 | −1.160 | 0.031 | −0.474 | −0.382 | −0.347 |
| OPN5-Tango | 1.172 | 1.249 | 0.015 | −2.310 | −0.286 | −1.200 | −0.107 | −0.113 |
| OPRD1-Tango | −0.020 | −0.058 | −0.228 | −1.962 | −0.308 | −1.218 | 0.098 | −0.125 |
| OPRK1-Tango | 0.020 | −0.074 | 0.538 | −1.777 | 0.006 | −0.630 | 0.000 | −0.152 |
| OPRL1-Tango | 0.032 | −0.213 | −0.261 | −1.152 | −0.405 | −0.575 | 0.505 | 0.420 |
| OPRM1-Tango | 0.333 | 0.031 | 0.886 | −1.267 | 0.577 | −0.617 | −0.291 | 0.000 |
| OXER1-Tango | 0.489 | 0.614 | 0.644 | −1.392 | −0.260 | −1.331 | −0.861 | −0.348 |
| OXGR1-Tango | 0.335 | 0.203 | 0.094 | −2.241 | −0.174 | −1.158 | −0.488 | −0.280 |
| OXTR-Tango | 0.145 | 0.213 | 0.000 | −0.850 | 0.180 | −0.149 | 0.034 | 0.000 |
| P2RY1-Tango | 0.200 | 0.233 | 0.315 | −2.084 | −0.206 | −0.173 | −0.054 | −0.107 |
| P2RY10-Tango | −0.041 | −0.145 | 0.121 | −1.635 | −0.132 | −0.616 | −0.015 | −0.392 |
| P2RY11-Tango | 0.331 | −0.075 | 0.140 | −2.242 | −0.261 | −0.033 | −0.237 | −0.627 |
| P2RY12-Tango | 0.684 | 0.678 | 0.630 | −1.816 | −0.060 | −1.005 | 0.535 | 0.768 |
| P2RY13-Tango | 0.293 | 0.200 | −0.110 | −0.915 | −0.392 | −0.400 | 0.167 | 0.275 |
| P2RY14-Tango | 0.382 | 0.204 | −0.340 | −0.999 | −0.710 | −0.609 | 0.041 | 0.338 |
| P2RY2-Tango | 0.218 | 0.059 | −0.261 | −1.691 | −0.483 | −0.334 | −0.145 | −0.150 |
| P2RY4-Tango | −0.021 | −0.073 | −0.148 | −2.040 | −0.886 | −0.761 | −0.027 | 0.007 |
| P2RY6-Tango | 0.080 | 0.144 | −0.362 | −1.877 | −0.196 | −0.600 | 0.240 | 0.165 |
| P2RY8-Tango | −0.149 | −0.125 | −0.234 | −1.669 | −0.827 | −0.917 | 0.007 | 0.074 |
| PK1-Tango | −0.312 | −0.459 | −0.491 | −1.302 | −0.267 | −0.811 | −0.089 | −0.162 |
| PK2-Tango | 0.339 | 0.220 | −0.247 | −0.799 | −0.262 | −0.255 | −0.573 | −0.376 |
| PRRP-Tango | 0.304 | 0.088 | −0.707 | −1.325 | −0.274 | −0.038 | 0.436 | 0.242 |
| PTAFR-Tango | 0.385 | 0.383 | 0.096 | −1.303 | −0.307 | −0.114 | 0.449 | 0.646 |
| PTGDR-Tango | −0.183 | −0.446 | −0.291 | −1.682 | −0.072 | 0.076 | 0.017 | −0.017 |
| PTGER1-Tango | −0.022 | −0.515 | −0.087 | −1.410 | 0.167 | 0.092 | −0.170 | −0.122 |
| PTGER2-Tango | −0.155 | −0.026 | −0.391 | −1.328 | −0.055 | −0.336 | 0.698 | 0.397 |
| PTGER3-Tango | 0.146 | 0.348 | 0.023 | −1.478 | −0.212 | −0.126 | 0.598 | 0.654 |
| PTGER4-Tango | 0.008 | 0.092 | 0.197 | −1.536 | 0.317 | 0.048 | 0.296 | 0.204 |
| PTGFR-Tango | −0.155 | −0.045 | −0.338 | −2.019 | −0.085 | −0.106 | 0.405 | −0.127 |
| PTGIR-Tango | −0.113 | −0.291 | −0.348 | −1.193 | −0.450 | −0.057 | 0.545 | 0.697 |
| PTH1R-Tango | −0.125 | −0.376 | −0.329 | −1.147 | −0.122 | −0.461 | 0.650 | 0.671 |
| PTH2R-Tango | −0.072 | −0.563 | −0.746 | −1.199 | −0.832 | −0.573 | 0.379 | 0.308 |
| QRFP-Tango | −0.227 | −0.546 | −0.140 | −1.669 | −0.140 | 0.000 | 0.353 | 0.092 |
| RXFP1-Tango | −0.151 | −0.405 | −0.410 | −2.068 | −0.495 | −0.017 | −0.419 | −0.543 |
| RXFP2-Tango | −0.125 | −0.351 | −0.750 | −1.538 | −1.033 | −0.804 | −0.183 | −0.208 |
| RXFP3-Tango | −0.460 | −0.773 | −0.312 | −1.206 | −0.084 | −0.555 | −0.198 | −0.428 |
| RXFP4-Tango | 0.284 | 0.027 | −0.533 | −1.018 | −0.560 | 0.000 | 0.117 | 0.060 |
| S1PR1-Tango | 1.595 | 1.306 | −0.251 | −0.704 | −0.500 | 0.030 | 0.308 | 0.064 |
| S1PR2-Tango | 1.018 | 0.991 | −0.245 | −2.812 | −0.643 | 0.017 | 0.101 | −0.619 |
| S1PR3-Tango | 0.548 | 0.508 | −0.157 | −1.835 | −0.119 | 0.154 | 0.591 | −0.285 |
| S1PR4-Tango | 0.406 | 0.729 | −0.601 | −2.428 | −0.231 | −0.699 | 0.089 | −0.035 |
| S1PR5-Tango | −0.094 | 0.491 | −0.240 | −0.993 | −0.219 | −0.087 | 0.000 | 0.148 |
| SCTR-Tango | 0.435 | 0.000 | −0.081 | −1.490 | −0.136 | −0.305 | 0.807 | 0.951 |
| SSTR1-Tango | −0.710 | −0.252 | −0.766 | −1.541 | −0.241 | −0.286 | 0.462 | 0.263 |
| SSTR2-Tango | 0.939 | 1.072 | −0.754 | −1.368 | 0.026 | −0.660 | −1.000 | −0.333 |
| SSTR3-Tango | 0.258 | 0.827 | −0.129 | −1.406 | 0.052 | −0.243 | 0.745 | 0.623 |
| SSTR4-Tango | −0.537 | −0.208 | −0.939 | −0.097 | −0.042 | −0.554 | 0.601 | 0.722 |
| SSTR5-Tango | −0.359 | −0.033 | −0.657 | 0.730 | 0.614 | 0.665 | 0.639 | −0.111 |
| SUCNR1-Tango | −0.455 | −0.483 | −0.529 | 3.142 | 2.409 | 2.875 | 2.663 | 2.606 |
| TA1-Tango | 0.283 | 0.248 | −0.300 | −1.276 | −0.188 | −0.133 | 0.538 | 0.461 |
| TAAR2-Tango | 0.177 | 0.063 | −0.250 | −1.073 | −0.076 | −0.256 | 0.256 | −0.020 |
| TAAR5-Tango | −0.457 | −0.642 | −0.512 | −1.404 | −0.189 | −1.031 | 0.011 | −0.513 |
| TAAR6-Tango | −0.136 | −0.290 | −0.059 | −0.611 | −0.881 | −0.810 | −0.447 | −0.514 |
| TAAR8-Tango | −0.677 | −0.385 | −0.143 | −1.075 | −0.508 | −0.952 | −0.488 | −0.488 |
| TAAR9-Tango | −0.481 | −0.473 | 0.080 | −1.166 | −0.641 | −0.826 | −0.455 | −0.373 |
| TACR1-Tango | −0.012 | 0.144 | −0.092 | −0.559 | −0.255 | −0.344 | 0.191 | 0.300 |
| TACR2-Tango | 0.067 | −0.107 | −0.558 | −0.842 | −0.448 | −0.319 | −0.036 | −0.302 |
| TACR3-Tango | 0.331 | 0.194 | 0.074 | −0.842 | −0.104 | −0.467 | −0.165 | −0.206 |
| TBXA2R-Tango | −0.064 | −0.164 | −0.446 | −0.993 | −0.364 | −0.502 | −0.427 | −0.045 |
| TSHR-Tango | −0.427 | −0.184 | 0.073 | −1.295 | −0.660 | −1.062 | −0.933 | −0.619 |
| UTS2R-Tango | −0.243 | 0.136 | −0.262 | −1.064 | −0.125 | −0.445 | −0.909 | −1.364 |
| VIPR1-Tango | −0.031 | −0.236 | −0.299 | −1.059 | −0.453 | −1.604 | −1.074 | −0.448 |
| VIPR2-Tango | 1.700 | 2.072 | 0.556 | 0.778 | −0.112 | 0.731 | 1.840 | 2.165 |

| | | Morganella | Morganella | | | | | |
|---|---|---|---|---|---|---|---|---|
| | | morganii- | morganii- | (Enterobac- | (Enterobac- | (Enterobac- | (Enterobac- | (Enterobac- |
| PRESTO-Tango | Morganella | ATCC | ATCC | teria)UC/ | teria)UC/ | teria)UC/ | teria)UC/ | teria)UC/ |
| with GMM | spp-6# | 25830 | 49948 | UC-1# | UC-2# | UC-3# | UC-4# | UC-5# |
| ADCYAP1R1- | −0.371 | 0.026 | 0.093 | −0.937 | −1.444 | 0.108 | −0.892 | −0.549 |
| Tango | | | | | | | | |
| ADORA1-Tango | −0.116 | 0.085 | −0.037 | −1.873 | −2.027 | −0.896 | −1.359 | −1.677 |
| ADORA2A-Tango | 0.094 | −0.146 | −0.071 | −1.246 | −1.627 | −0.942 | −0.488 | −0.602 |
| ADORA2B-Tango | −0.364 | −0.155 | −0.342 | −0.982 | −1.525 | −0.295 | −0.820 | −0.858 |
| ADORA3-Tango | −0.376 | −0.125 | −0.265 | −0.837 | −1.150 | 0.560 | −0.670 | −0.431 |
| ADRA1A-Tango | −0.252 | −0.247 | −0.226 | −1.041 | −1.386 | −0.136 | −0.684 | −0.681 |
| ADRA1B-Tango | −0.555 | −0.545 | −1.010 | −1.143 | −1.643 | −0.648 | −0.775 | −0.942 |
| ADRA1D-Tango | −0.374 | −0.540 | −0.647 | −1.021 | −1.455 | −1.019 | −0.570 | −0.306 |
| ADRA2A-Tango | 1.760 | 1.651 | 1.806 | −3.386 | −3.281 | 0.249 | −1.144 | −0.892 |
| ADRA2B-Tango | 0.349 | −0.037 | 0.488 | −3.332 | −2.654 | −1.514 | −1.194 | −1.163 |
| ADRA2C-Tango | 1.361 | 0.618 | 1.167 | −1.405 | −1.515 | −0.121 | −0.267 | −0.531 |
| ADRB1-Tango | 0.461 | 1.040 | 1.302 | −1.875 | −1.497 | 0.379 | −0.954 | −0.585 |
| ADRB2-Tango | −0.239 | −0.103 | −0.425 | −1.712 | −2.332 | −0.304 | −0.563 | −1.813 |
| ADRB3-Tango | 0.423 | 0.353 | 0.334 | −1.153 | −1.203 | 0.129 | −0.987 | −0.670 |
| AGTR1-Tango | 0.429 | −0.012 | −0.027 | −1.375 | −1.826 | 0.230 | −1.164 | −0.990 |
| AGTR2-Tango | 0.045 | −0.143 | −0.001 | −1.233 | −1.298 | 0.012 | −1.506 | −0.770 |
| APJ-Tango | −0.222 | 0.009 | 0.294 | −1.961 | −1.843 | −0.931 | −1.364 | −1.237 |
| AVPR1A-Tango | −0.390 | 0.342 | 0.173 | −1.845 | −2.306 | −0.451 | −1.227 | −0.972 |
| AVPR1B-Tango | −0.005 | −0.255 | −0.358 | −1.485 | −1.780 | −0.192 | −1.298 | −0.670 |
| AVPR2-Tango | 0.031 | 0.021 | 0.059 | −2.189 | −2.077 | −0.189 | −1.769 | −1.197 |
| BB3-Tango | 0.037 | −0.077 | −0.083 | −1.286 | −1.429 | −0.549 | −1.336 | −0.473 |
| BDKBR1-Tango | 0.240 | −0.020 | 0.220 | −0.608 | −1.269 | −0.109 | −1.345 | −0.524 |
| BDKBR2-Tango | −0.013 | 0.784 | 0.467 | −0.567 | −0.897 | 0.081 | −1.048 | −0.257 |
| C3AR1-Tango | −0.296 | 0.214 | 0.360 | −0.940 | −1.174 | −0.327 | −1.472 | −1.216 |
| C5A-Tango | −0.185 | −0.309 | −0.318 | −0.924 | −1.600 | 0.079 | −1.026 | −0.571 |
| CALCRb-Tango | −0.634 | −0.087 | −0.222 | −0.894 | −1.368 | −0.359 | −0.448 | −0.736 |
| CALCRL-Tango | −0.148 | 0.202 | −0.294 | −0.655 | −1.012 | −0.166 | −0.288 | −0.397 |
| CASR-Tango | −0.456 | −0.785 | −1.061 | −0.886 | −1.158 | −0.367 | −0.805 | −0.648 |
| CCKAR-Tango | −0.296 | −0.118 | −0.261 | −0.563 | −0.735 | −0.138 | −0.578 | −0.405 |
| CCKBR-Tango | 0.226 | −0.100 | 0.318 | −1.024 | −1.291 | −0.499 | −0.725 | −0.847 |
| CCR10-Tango | −0.206 | 0.166 | 0.126 | −1.314 | −1.350 | −1.067 | −0.502 | −0.922 |
| CCR2-Tango | −0.805 | 0.056 | 0.000 | −1.344 | −1.268 | −0.803 | −0.362 | −0.588 |
| CCR3-Tango | −0.455 | −0.379 | −0.334 | −2.076 | −2.362 | −0.930 | −1.973 | −1.273 |
| CCR4-Tango | −0.744 | −0.666 | −0.831 | −1.440 | −1.845 | −0.076 | −0.947 | −0.666 |
| CCR5-Tango | −0.384 | −0.068 | −0.533 | −1.350 | −1.459 | 0.153 | −0.844 | −0.163 |
| CCR6-Tango | 0.756 | 0.141 | 0.260 | −0.729 | −1.535 | 0.248 | −1.282 | −0.896 |
| CCR7-Tango | −0.549 | −0.031 | −0.206 | −1.399 | −1.903 | −0.098 | −1.203 | −0.328 |
| CCR8-Tango | −0.502 | −0.767 | −0.375 | −1.906 | −2.741 | −0.076 | −0.958 | −0.360 |
| CCRL2-Tango | 1.096 | 0.000 | 0.728 | −1.382 | −1.774 | 0.536 | −0.493 | −0.707 |
| CD97-Tango | 1.045 | −0.261 | 0.094 | −1.455 | −1.673 | −0.255 | −1.000 | −0.639 |
| CHRM1-Tango | 0.496 | 0.060 | 0.198 | −1.106 | −1.232 | −0.096 | −0.372 | −0.310 |
| CHRM2-Tango | 0.532 | 0.168 | 0.354 | −1.170 | −1.041 | 0.007 | −1.012 | −0.254 |
| CHRM3-Tango | 0.497 | −0.071 | 0.382 | −1.370 | −1.776 | 0.173 | −1.064 | −0.609 |
| CHRM4-Tango | 0.120 | −0.069 | −0.048 | −0.708 | −1.373 | −0.012 | −0.642 | −0.773 |
| CHRM5-Tango | −0.110 | 0.007 | 0.000 | −0.938 | −1.106 | 0.048 | −0.574 | −0.234 |
| CMKLR1-Tango | −0.058 | 0.022 | 0.055 | −1.417 | −1.641 | 0.259 | −0.674 | −0.773 |
| CNR1-Tango | −0.207 | −0.455 | −0.618 | −1.413 | −1.543 | −0.233 | −1.105 | −0.454 |
| CNR2-Tango | −0.155 | 0.326 | −0.064 | −1.327 | −1.882 | −0.020 | −1.084 | −0.720 |
| CRHR1-Tango | −0.292 | 0.100 | 0.100 | −1.801 | −2.005 | −0.229 | −0.480 | −0.480 |
| CRHR2-Tango | −0.028 | −0.461 | −0.304 | −0.989 | −1.211 | 0.228 | −0.889 | −0.659 |
| CX3C1-Tango | −0.728 | 0.664 | 0.528 | −1.160 | −1.482 | −0.750 | −0.792 | −1.196 |
| CXCR1-Tango | 0.902 | 0.125 | 0.305 | −1.451 | −1.729 | −0.515 | −0.160 | −0.033 |
| CXCR2-Tango | 1.691 | 1.101 | 1.282 | −0.699 | −1.775 | −0.087 | −0.278 | −0.048 |
| CXCR3-Tango | 0.457 | 0.043 | 0.169 | −1.621 | −2.372 | −0.339 | −0.786 | −0.990 |
| CXCR4-Tango | 0.121 | 0.189 | 0.204 | −0.924 | −1.309 | −0.083 | −0.405 | −0.913 |
| CXCR5-Tango | 0.357 | 0.172 | 0.447 | −0.930 | −1.580 | 0.276 | −0.714 | −1.065 |
| CXCR6-Tango | 0.244 | 0.500 | 1.019 | −0.555 | −1.519 | −0.231 | −0.819 | −0.434 |
| CXCR7-Tango | 0.449 | 0.418 | 0.616 | −1.593 | −1.282 | −0.737 | −1.744 | −0.566 |
| CYSLTR1-Tango | 1.076 | 0.403 | 0.505 | −1.551 | −1.996 | −0.750 | −0.478 | 0.460 |
| CYSLTR2-Tango | 0.655 | −0.017 | 0.142 | −1.155 | −1.722 | −0.325 | −0.037 | 0.193 |
| DRD1-Tango | 0.564 | 1.913 | 1.759 | −2.292 | −3.201 | −0.956 | −1.349 | −0.676 |
| DRD2-Tango | 3.630 | 4.089 | 4.073 | −2.278 | −2.826 | −0.045 | −1.922 | −0.869 |
| DRD3-Tango | 3.863 | 2.887 | 4.096 | −1.863 | −2.326 | 0.385 | −0.387 | −0.983 |
| DRD4-Tango | 2.678 | 2.627 | 3.559 | −2.415 | −2.065 | 0.455 | −1.200 | −1.087 |
| DRD5-Tango | −0.560 | 0.007 | 0.007 | −1.411 | −1.748 | −1.367 | −0.672 | −0.624 |
| EDNRA-Tango | −0.443 | −0.129 | −0.349 | −0.990 | −1.298 | −1.596 | −0.548 | −0.244 |
| EDNRB-Tango | 0.401 | 0.133 | −0.198 | −0.837 | −1.352 | −0.897 | −0.487 | −0.281 |
| ELTD1-Tango | −0.120 | −0.712 | −0.643 | −1.052 | −1.208 | −1.361 | −0.959 | −0.446 |
| F2R-Tango | −0.087 | −0.101 | −0.345 | −1.338 | −1.776 | −0.944 | −0.890 | −0.675 |
| F2RL1-Tango | −0.302 | −0.197 | −0.562 | −0.872 | −1.313 | −0.742 | −0.712 | −0.705 |
| F2RL2-Tango | −0.331 | −0.558 | −0.631 | −0.880 | −1.372 | −0.632 | −0.953 | −0.694 |
| F2RL3-Tango | −0.342 | −0.362 | −0.445 | −1.287 | −1.600 | −1.028 | −0.996 | −0.829 |
| FFA1-Tango | 0.268 | 0.276 | 0.355 | −0.944 | −0.814 | −0.535 | −0.804 | −0.159 |
| FFA2-Tango | 0.471 | −0.079 | 0.241 | −1.454 | −1.798 | −0.495 | −0.807 | −0.505 |
| FFA3-Tango | 0.027 | 0.035 | 0.027 | −1.150 | −1.418 | −0.118 | −0.597 | −0.654 |
| FPR1-Tango | 1.057 | 0.787 | 0.921 | −0.533 | −0.448 | −1.413 | −0.508 | 0.097 |
| FPR2-Tango | 0.802 | 0.344 | 0.347 | −1.074 | −1.541 | −0.251 | −0.357 | −0.090 |
| FPR3-Tango | 0.135 | −0.090 | 0.159 | −1.208 | −1.293 | −0.608 | −0.780 | −0.378 |
| FSHR-Tango | −0.208 | −0.315 | −0.416 | −0.656 | −0.945 | −0.360 | −0.563 | −0.109 |
| GABBR1-Tango | 1.022 | 0.225 | 1.355 | −0.490 | −0.896 | −0.350 | −0.731 | −0.484 |
| GAL1-Tango | 0.160 | 0.261 | 0.712 | −2.705 | −2.395 | −1.293 | −1.235 | −0.913 |
| GAL2-Tango | −0.853 | −0.252 | 0.320 | −1.845 | −1.812 | −0.840 | −1.534 | −1.031 |
| GAL3-Tango | −0.391 | −0.873 | −0.671 | −1.382 | −1.239 | −0.739 | −1.090 | −1.142 |
| GCGR-Tango | −0.547 | 0.210 | 0.284 | −1.359 | −1.690 | −0.413 | −0.523 | −0.778 |
| GHRHR-Tango | −0.771 | 0.325 | 0.288 | −1.948 | −1.990 | −0.313 | −1.070 | −1.119 |
| GHSR-Tango | −0.683 | −0.004 | 0.226 | −1.751 | −1.710 | −1.270 | −1.738 | −1.525 |
| GIPR-Tango | −1.076 | −0.035 | 0.063 | −1.493 | −2.076 | −1.103 | −1.336 | −0.969 |
| GLP1R-Tango | −1.332 | −0.631 | −0.841 | −1.974 | −2.572 | −0.915 | −1.775 | −1.157 |
| GLP2R-Tango | 0.027 | −0.021 | −0.134 | −2.178 | −1.757 | −0.143 | −0.843 | −0.553 |
| GNRHR-Tango | −0.053 | 0.202 | 0.267 | −1.377 | −1.555 | 0.086 | −0.374 | −0.667 |
| GPBA-Tango | 0.677 | −0.304 | 0.019 | −1.423 | −1.556 | 0.026 | −0.436 | −0.792 |
| GPER-Tango | 0.218 | 0.020 | 0.371 | −1.244 | −1.737 | −0.246 | −0.810 | −0.501 |
| GPR1-Tango | −0.232 | 0.192 | 0.057 | −1.462 | −2.014 | −0.343 | −1.000 | −0.364 |
| GPR101-Tango | 0.304 | 0.287 | 0.118 | −0.840 | −0.823 | −0.271 | −0.324 | 0.233 |
| GPR110-Tango | −0.524 | −0.638 | −0.576 | −0.918 | −1.029 | −0.573 | −1.142 | −0.575 |
| GPR111-Tango | 0.016 | −0.215 | −0.135 | −0.916 | −1.307 | −0.504 | −1.098 | −0.577 |
| GPR113-Tango | −0.487 | −0.298 | −0.368 | −1.559 | −1.660 | −0.751 | −0.709 | −0.592 |
| GPR114-Tango | −0.196 | −0.314 | −0.244 | −0.665 | −1.296 | −0.312 | −0.287 | −0.325 |
| GPR115-Tango | −0.467 | −0.475 | −0.507 | −0.689 | −1.002 | −0.499 | −0.658 | −0.105 |
| GPR116-Tango | 0.059 | −0.230 | −0.149 | −1.042 | −1.529 | −0.534 | −1.162 | −0.839 |
| GPR119-Tango | 0.184 | 0.249 | 0.198 | −1.059 | −1.593 | −0.562 | −0.448 | −0.201 |
| GPR12-Tango | 0.150 | 0.548 | 0.772 | −1.226 | −1.697 | −0.448 | −1.039 | −0.350 |
| GPR120-Tango | 0.740 | 0.086 | 0.605 | −0.809 | −1.565 | 0.204 | −0.417 | −0.681 |
| GPR123-Tango | −0.045 | 0.366 | 0.165 | −0.908 | −1.248 | −0.088 | −0.959 | −0.279 |
| GPR124-Tango | −0.085 | −0.543 | −0.388 | −1.375 | −1.469 | −0.173 | −1.180 | −0.531 |
| GPR125-Tango | 0.072 | −0.658 | −0.806 | −0.456 | −0.668 | −0.340 | −0.810 | −0.429 |
| GPR126-Tango | 0.165 | 0.097 | −0.065 | −0.892 | −1.408 | −0.648 | −1.084 | −0.853 |
| GPR132-Tango | −0.462 | −0.837 | −0.877 | −0.180 | −0.792 | −0.421 | −1.458 | −1.065 |
| GPR133-Tango | −0.123 | −0.326 | −0.376 | −0.442 | −0.907 | −0.078 | −0.906 | −0.667 |
| GPR135-Tango | −0.371 | 0.153 | 0.170 | −0.636 | −1.161 | −0.523 | −0.975 | −0.858 |
| GPR141-Tango | 0.445 | 0.124 | 0.415 | −0.748 | −0.609 | 0.247 | −1.325 | −1.515 |
| GPR142-Tango | −0.200 | −0.459 | −0.476 | −1.851 | −2.695 | −0.483 | −1.976 | −1.535 |
| GPR143-Tango | −0.861 | −1.073 | −1.370 | −2.461 | −2.461 | −0.306 | −1.055 | −0.544 |
| GPR144-Tango | −0.691 | −0.705 | −1.196 | −1.850 | −2.765 | −0.662 | −1.040 | −0.827 |
| GPR146-Tango | −0.523 | −0.369 | −0.528 | −1.991 | −2.935 | 0.204 | −1.186 | −1.298 |
| GPR148-Tango | 0.766 | −0.127 | −0.658 | −1.501 | −2.446 | −0.503 | −2.158 | −1.771 |
| GPR149-Tango | 0.966 | −0.902 | −0.855 | −2.038 | −2.809 | −0.390 | −1.016 | 0.046 |
| GPR15-Tango | −0.347 | 0.074 | 0.160 | −1.811 | −2.223 | −0.474 | −0.952 | −0.310 |
| GPR150-Tango | 0.176 | 0.369 | 0.170 | −0.702 | −1.271 | −0.145 | −0.675 | −1.203 |
| GPR151-Tango | 0.813 | −0.627 | −0.478 | −1.550 | −1.902 | −0.227 | −0.886 | 0.178 |
| GPR152-Tango | −0.290 | −0.200 | −0.282 | −0.914 | −0.998 | −0.238 | −0.981 | −0.503 |
| GPR153-Tango | 0.086 | −0.099 | −0.442 | −0.794 | −1.035 | −0.254 | −0.986 | −0.240 |
| GPR156-Tango | 0.337 | 0.099 | 0.247 | −0.470 | −0.887 | 0.276 | −1.016 | −0.377 |
| GPR157-Tango | 0.490 | −0.175 | −0.154 | −1.010 | −1.803 | −0.145 | −0.984 | −0.662 |
| GPR158-Tango | −0.202 | −0.093 | −0.434 | −0.762 | −1.225 | −0.092 | −0.546 | −0.461 |
| GPR160-Tango | 0.083 | −0.271 | −0.330 | −0.608 | −2.772 | 0.467 | −0.349 | 0.129 |
| GPR161-Tango | 0.328 | −0.399 | −0.345 | −0.315 | −0.635 | −0.016 | −0.796 | −0.350 |
| GPR162-Tango | −0.406 | −0.412 | −0.483 | −0.686 | −1.123 | −0.496 | −0.894 | −0.749 |
| GPR17-Tango | 0.656 | −0.446 | −0.516 | −1.029 | −0.982 | −0.074 | −0.669 | 0.121 |
| GPR171-Tango | 0.386 | −0.279 | −0.380 | −1.099 | −1.377 | 0.478 | −0.814 | −0.400 |
| GPR173-Tango | 0.176 | 0.013 | −0.290 | −1.305 | −1.265 | 0.015 | −0.140 | −0.906 |
| GPR174-Tango | −0.116 | −0.144 | −0.186 | −1.311 | −1.390 | −0.627 | −0.589 | −0.623 |
| GPR18-Tango | 0.047 | −0.228 | −0.093 | −1.174 | −1.368 | −0.544 | −0.834 | −0.866 |
| GPR182-Tango | 0.013 | −0.584 | −0.631 | −0.836 | −1.007 | −0.378 | −0.742 | −0.459 |
| GPR183-Tango | −0.411 | −0.089 | 0.052 | −1.105 | −1.618 | −0.283 | −0.635 | −0.698 |
| GPR19-Tango | −0.493 | −0.895 | −1.076 | −0.936 | −1.061 | −0.480 | −0.966 | −0.493 |
| GPR20-Tango | 0.605 | 0.200 | 0.000 | −0.808 | −1.514 | −0.491 | −0.866 | −0.463 |
| GPR21-Tango | 0.148 | 0.509 | 0.133 | −1.073 | −1.262 | −0.170 | −0.675 | −0.046 |
| GPR22-Tango | −0.042 | −0.394 | −0.405 | −0.683 | −0.962 | 0.095 | −0.659 | −0.087 |
| GPR25-Tango | 0.000 | −0.197 | −0.485 | −1.073 | −1.515 | −0.245 | −0.604 | −0.518 |
| GPR26-Tango | 0.424 | 0.522 | 0.636 | −1.946 | −2.172 | −0.959 | −1.098 | −0.759 |
| GPR27-Tango | 0.143 | −0.112 | −0.028 | −1.018 | −1.002 | 0.089 | −0.795 | −0.206 |
| GPR3-Tango | 0.567 | 0.267 | 0.408 | −1.057 | −0.977 | −0.733 | −0.745 | −0.083 |
| GPR31-Tango | 0.078 | 0.048 | 0.253 | −1.704 | −2.307 | −0.864 | −1.477 | −0.581 |
| GPR32-Tango | −0.259 | −0.076 | 0.110 | −1.633 | −1.592 | −1.093 | −0.628 | −0.628 |
| GPR34-Tango | 0.284 | 0.239 | 0.424 | −1.580 | −1.875 | −0.446 | −0.945 | −0.684 |
| GPR35-Tango | −0.080 | −0.014 | 0.005 | −1.701 | −1.766 | −0.832 | −1.338 | −0.431 |
| GPR37-Tango | 0.061 | 0.011 | −0.070 | −1.182 | −1.446 | −0.237 | −1.346 | −0.701 |
| GPR37L1-Tango | −0.110 | −0.169 | −0.117 | −1.336 | −1.653 | −0.099 | −1.182 | −0.622 |
| GPR39-Tango | −0.260 | −0.096 | −0.220 | −1.003 | −1.602 | −0.461 | −0.958 | −0.872 |
| GPR4-Tango | −0.313 | −0.115 | −0.064 | −0.620 | −0.948 | −0.409 | −1.073 | −0.676 |
| GPR44-Tango | −0.166 | 0.130 | 0.099 | −0.774 | −1.367 | −0.352 | −0.908 | −0.677 |
| GPR45-Tango | −0.624 | −0.119 | 0.340 | −1.462 | −1.416 | −0.399 | −0.650 | −0.523 |
| GPR50-Tango | 0.218 | 0.114 | −0.085 | −1.748 | −1.679 | 0.100 | −0.850 | 0.090 |
| GPR52-Tango | 0.459 | 0.496 | 0.777 | −1.174 | −1.399 | 0.154 | −0.930 | −0.269 |
| GPR55-Tango | 0.440 | −0.040 | 0.097 | −1.513 | −1.563 | 0.377 | −1.138 | −0.307 |
| GPR56-Tango | 0.080 | 0.024 | 0.097 | −1.103 | −1.765 | −0.431 | −1.499 | −1.059 |
| GPR6-Tango | 0.151 | −0.036 | 0.078 | −0.995 | −1.362 | −1.077 | −0.471 | −0.012 |
| GPR61-Tango | −0.168 | −0.024 | 0.093 | −1.012 | −1.255 | −1.213 | −0.838 | 0.014 |
| GPR62-Tango | −0.015 | 0.133 | 0.125 | −0.727 | −1.103 | −0.068 | −0.643 | −0.295 |
| GPR63-Tango | −0.130 | −0.356 | −0.301 | −0.335 | −0.252 | 0.041 | −0.187 | −0.081 |
| GPR64-Tango | −0.148 | −0.155 | −0.040 | −0.696 | −0.945 | −0.189 | −0.443 | −0.361 |
| GPR65-Tango | −0.141 | −0.192 | −0.100 | −0.597 | −0.668 | −0.172 | −0.312 | 0.002 |
| GPR68-Tango | −0.501 | −0.421 | −0.327 | −1.125 | −1.380 | −0.502 | −0.998 | −0.899 |
| GPR75-Tango | −0.291 | −0.155 | −0.105 | −0.774 | −1.200 | 0.182 | −0.362 | −0.521 |
| GPR77-Tango | −0.473 | −0.428 | −0.246 | −1.730 | −2.039 | −0.219 | −0.945 | −0.753 |
| GPR78-Tango | 0.141 | −0.150 | 0.025 | −1.266 | −1.792 | −0.527 | −0.290 | 0.124 |
| GPR82-Tango | −0.347 | −0.123 | 0.063 | −1.640 | −1.862 | −0.970 | −1.356 | −0.994 |
| GPR83-Tango | −0.634 | −0.571 | −0.450 | −1.015 | −1.501 | −0.344 | −1.228 | −0.920 |
| GPR84-Tango | −0.090 | −0.225 | −0.080 | −1.164 | −1.129 | −0.205 | −1.297 | −0.717 |
| GPR85-Tango | 0.128 | 0.094 | 0.031 | −1.003 | −1.468 | −0.049 | −1.177 | −0.516 |
| GPR87-Tango | −0.043 | 0.052 | 0.076 | −0.731 | −0.967 | −0.137 | −0.632 | −0.476 |
| GPR88-Tango | 0.162 | 0.111 | 0.182 | −0.869 | −1.491 | −0.041 | −0.564 | −0.610 |
| GPR97-Tango | 0.056 | −0.262 | −0.201 | −0.796 | −1.030 | 0.037 | −0.717 | −0.336 |
| GPRC5A-Tango | −0.144 | −0.193 | −0.086 | −1.019 | −1.205 | −0.142 | −1.311 | −0.826 |
| GPRC5B-Tango | −0.155 | −0.459 | −0.417 | −0.787 | −0.901 | −0.068 | −1.143 | −0.446 |
| GPRC5C-Tango | −0.342 | −0.510 | −0.714 | −0.602 | −0.849 | 0.099 | −0.670 | −0.109 |
| GPRC5D-Tango | −0.146 | −0.347 | −0.429 | −0.811 | −1.153 | −0.049 | −0.894 | −0.878 |
| GPRC6A-Tango | −0.472 | −0.442 | −0.757 | −0.789 | −0.887 | 0.057 | −1.006 | −0.419 |
| GRM1-Tango | −0.441 | −0.284 | −0.182 | −0.632 | −0.863 | −0.284 | −1.040 | −0.688 |
| GRM2-Tango | −0.112 | −0.415 | −0.212 | −0.866 | −1.157 | −0.203 | −0.789 | −0.762 |
| GRM4-Tango | −0.695 | −0.616 | −0.431 | −0.510 | −0.749 | −0.157 | −0.790 | −0.334 |
| GRM5-Tango | −0.520 | −0.351 | −0.752 | −0.116 | −0.060 | −0.284 | −0.794 | −0.220 |
| GRM6-Tango | −0.385 | −0.285 | −0.465 | −1.510 | −1.404 | −0.430 | −1.048 | −0.567 |
| GRM7-Tango | −0.798 | −0.585 | −0.641 | −0.921 | −1.287 | −0.759 | −1.274 | −0.217 |
| GRM8-Tango | −0.100 | −0.511 | −0.250 | −1.134 | −1.561 | −0.662 | −1.443 | −0.696 |
| GRPR-Tango | −0.675 | 0.051 | −0.153 | −1.309 | −2.068 | 0.161 | −1.659 | −0.925 |
| HCA1-Tango | −0.076 | −0.352 | 0.000 | −1.363 | −2.254 | 0.109 | −0.758 | −1.363 |
| HCA2-Tango | 0.444 | −0.685 | −0.785 | −1.040 | −1.378 | −0.012 | −1.003 | −0.674 |
| HCA3-Tango | 0.100 | −0.482 | −0.520 | −1.355 | −1.832 | −0.359 | −1.042 | −0.473 |
| HCTR1-Tango | −0.089 | 0.189 | −0.078 | −1.228 | −2.079 | −0.100 | −1.138 | −1.250 |
| HCTR2-Tango | −0.127 | 0.000 | −0.093 | −1.217 | −1.552 | −0.585 | −0.545 | −0.545 |
| HRH1-Tango | 0.535 | −0.595 | −0.542 | −1.425 | −1.510 | −0.708 | −1.297 | −0.273 |
| HRH2-Tango | 0.634 | 0.233 | 0.560 | −0.868 | −1.317 | 0.138 | −0.820 | −0.010 |
| HRH3-Tango | 1.883 | 1.289 | 1.318 | −0.295 | −1.260 | 0.214 | −0.053 | 0.343 |
| HRH4-Tango | 1.912 | 1.740 | 1.631 | −0.096 | −0.901 | −0.241 | −0.638 | 0.056 |
| HTR1A-Tango | 0.583 | 0.068 | 0.756 | −0.856 | −1.621 | −0.037 | −1.213 | −0.512 |
| HTR1B-Tango | 0.157 | −0.480 | −0.366 | −0.998 | −1.285 | −0.524 | −1.281 | −0.381 |
| HTR1D-Tango | 0.419 | −0.139 | −0.102 | −1.232 | −1.526 | −0.673 | −1.235 | −0.553 |
| HTR1E-Tango | 0.149 | −0.282 | −0.325 | −0.963 | −1.146 | −0.179 | −1.217 | −0.403 |
| HTR1F-Tango | −0.314 | −0.274 | −0.136 | −1.980 | −1.475 | −0.480 | −1.146 | −0.753 |
| HTR2A-Tango | −0.145 | −0.702 | −0.779 | −1.055 | −2.038 | −0.461 | −0.950 | −0.915 |
| HTR2B-Tango | −0.072 | −0.698 | −0.627 | −0.900 | −1.067 | −0.293 | −0.768 | −0.405 |
| HTR2C-Tango | 0.176 | −0.703 | −0.389 | −1.525 | −2.147 | −0.547 | −0.980 | −0.809 |
| HTR4-Tango | −0.281 | 0.347 | 0.324 | −0.426 | −1.472 | −0.732 | −0.682 | 0.099 |
| HTR5-Tango | −0.222 | 0.186 | 0.308 | −0.934 | −1.157 | −0.541 | −1.462 | −1.202 |
| HTR6-Tango | 0.098 | −0.810 | −0.475 | −0.169 | −1.099 | −0.293 | −0.860 | −0.608 |
| HTR7-Tango | 0.417 | 0.045 | 0.632 | 0.791 | −0.661 | 0.079 | −1.069 | −0.283 |
| KISSPEPTIN-R- | 0.196 | 0.181 | 1.063 | −1.145 | −1.525 | −0.689 | −0.520 | −0.094 |
| Tango | | | | | | | | |
| LHCGR-Tango | −0.436 | −0.835 | −1.011 | −1.042 | −1.432 | −0.589 | −1.114 | −0.870 |
| LPAR1-Tango | 0.207 | −0.087 | −0.262 | −1.110 | −1.505 | −0.501 | −0.690 | −0.781 |
| LPAR2-Tango | −0.079 | 0.058 | −0.245 | −1.204 | −1.654 | −0.622 | −1.117 | −0.903 |
| LPAR4-Tango | −0.073 | −0.098 | −0.256 | −1.683 | −1.545 | −1.716 | −1.153 | −1.153 |
| LPAR5-Tango | 0.079 | −0.575 | −0.318 | −1.094 | −1.392 | −0.906 | −1.033 | −1.021 |
| LPAR6-Tango | 0.087 | −0.425 | −0.519 | −0.555 | −1.030 | −0.240 | −1.185 | −0.804 |
| LTB4R-Tango | −0.178 | −0.520 | −0.340 | −0.928 | −1.509 | −0.075 | −0.900 | −0.949 |
| LTB4R2B-Tango | 0.163 | −0.984 | −0.833 | −0.922 | −1.384 | −0.509 | −0.723 | −0.519 |
| MAS1-Tango | 0.630 | 0.105 | 0.181 | −1.349 | −1.752 | −0.575 | −0.769 | −0.344 |
| MAS1L-Tango | 0.422 | −0.634 | −0.818 | −0.841 | −1.327 | −0.262 | −0.669 | −0.498 |
| MC1R-Tango | 0.589 | −0.551 | −0.539 | −0.837 | −1.458 | −0.135 | −0.472 | −0.472 |
| MC2R-Tango | 0.069 | −0.206 | 0.000 | −1.029 | −1.550 | −0.247 | 0.229 | 0.413 |
| MC3R-Tango | 0.175 | −0.834 | −0.504 | −1.022 | −1.412 | 0.115 | −0.304 | −0.264 |
| MC4R-Tango | 0.602 | 0.000 | 0.532 | −1.484 | −1.822 | −0.506 | −0.541 | −1.167 |
| MC5R-Tango | 0.869 | 0.449 | 0.939 | −1.777 | −2.139 | −1.100 | −1.463 | −1.087 |
| MCHR1-Tango | −0.707 | −0.666 | −0.880 | −1.409 | −1.869 | −0.167 | −1.472 | −0.782 |
| MCHR2-Tango | −0.094 | −0.915 | −0.992 | −1.230 | −1.739 | −0.145 | −0.695 | −1.100 |
| MLNR-Tango | 0.445 | −0.077 | −0.216 | −0.914 | −1.243 | 0.052 | −0.615 | −0.597 |
| MRGPRD-Tango | −0.164 | −0.689 | −0.694 | −1.125 | −1.309 | −0.229 | −1.120 | −0.307 |
| MRGPRE-Tango | 0.214 | −0.175 | 0.036 | −0.605 | −0.987 | 0.330 | −0.462 | −0.531 |
| MRGPRF-Tango | 0.079 | 0.293 | 0.326 | −0.531 | −1.094 | 0.276 | −0.608 | −0.397 |
| MRGPRG-Tango | −0.008 | −0.288 | −0.170 | −0.035 | −0.111 | 0.249 | −0.482 | −0.142 |
| MRGPRX1-Tango | −0.037 | −0.473 | 0.108 | −0.836 | −1.283 | −0.191 | −1.563 | −1.288 |
| MRGPRX2-Tango | −0.101 | −0.660 | −0.582 | −1.647 | −1.448 | 0.049 | −0.772 | −0.461 |
| MRGPRX3-Tango | −0.106 | −0.426 | −0.300 | −1.371 | −1.991 | −0.163 | −1.125 | −1.033 |
| MRGPRX4-Tango | −0.654 | −0.720 | −1.138 | −2.281 | −2.738 | −1.216 | −2.033 | −1.854 |
| MTNR1A-Tango | 0.064 | 0.263 | 0.354 | −2.107 | −2.164 | −0.425 | −2.050 | −1.102 |
| MTNR1B-Tango | 0.103 | −0.186 | 0.639 | −2.001 | −2.390 | −0.770 | −1.166 | −0.634 |
| NMBR-Tango | −0.603 | −0.365 | −0.171 | −1.257 | −1.579 | 0.249 | −0.054 | −0.143 |
| NMUR1-Tango | −0.888 | 0.000 | −0.160 | −1.476 | −1.940 | −0.691 | −0.930 | −1.507 |
| NMUR2-Tango | −0.881 | −0.835 | −0.629 | −1.497 | −1.364 | −0.295 | −2.108 | −1.966 |
| NPBW1-Tango | 0.597 | 0.437 | 0.532 | −1.657 | −1.038 | −0.228 | −0.675 | −0.420 |
| NPBW2-Tango | 0.541 | 0.000 | 0.424 | −1.186 | −1.067 | −0.637 | −1.439 | −0.194 |
| NPFF1-Tango | 0.439 | 0.266 | 0.200 | −0.998 | −1.131 | 0.519 | −0.611 | −0.815 |
| NPFF2-Tango | −0.519 | −0.274 | −0.497 | −1.434 | −1.454 | −0.212 | −0.990 | −0.301 |
| NPS-Tango | −0.452 | −0.535 | −0.679 | −1.234 | −1.589 | −0.232 | −1.741 | −0.744 |
| NPY1R-Tango | −0.126 | −0.750 | −0.474 | −1.237 | −1.381 | −0.518 | −1.239 | 0.097 |
| NPY2R-Tango | −0.272 | −0.580 | −0.351 | −0.807 | −0.990 | 0.418 | −0.856 | −0.159 |
| NPY4R-Tango | −0.142 | 0.188 | 0.249 | −0.926 | −1.348 | −0.110 | −1.703 | −0.509 |
| NPY5R-Tango | −0.051 | −0.446 | −0.312 | −1.109 | −1.596 | 0.262 | −0.656 | −0.613 |
| NTSR1-Tango | 0.005 | −0.218 | −0.200 | −1.214 | −1.198 | −0.654 | −0.144 | −0.083 |
| NTSR2-Tango | 0.196 | −0.324 | −0.462 | −1.371 | −1.830 | −0.711 | −0.504 | −0.644 |
| OPN3-Tango | 0.028 | 0.120 | 0.000 | −1.182 | −1.614 | −0.229 | −0.065 | −0.411 |
| OPN5-Tango | −0.171 | −0.679 | −0.525 | −1.580 | −1.939 | −1.036 | −0.534 | −0.111 |
| OPRD1-Tango | −0.201 | −0.283 | −0.465 | −1.560 | −2.095 | −0.689 | −1.169 | −1.068 |
| OPRK1-Tango | 0.509 | −0.291 | −0.327 | −0.828 | −0.933 | −0.348 | −0.935 | −0.545 |
| OPRL1-Tango | −0.048 | 0.089 | −0.411 | −0.870 | −1.173 | −0.193 | −0.892 | −0.922 |
| OPRM1-Tango | −0.150 | −0.374 | 0.070 | −1.201 | −1.544 | −0.518 | −0.367 | −0.647 |
| OXER1-Tango | −0.084 | −0.470 | −0.822 | −0.801 | −1.156 | −0.061 | −0.358 | −0.294 |
| OXGR1-Tango | −0.469 | −0.645 | −0.510 | −2.202 | −2.582 | −0.912 | −1.250 | −1.117 |
| OXTR-Tango | 0.281 | −0.117 | −0.193 | −0.960 | −1.573 | −0.194 | −0.218 | −0.067 |
| P2RY1-Tango | 0.185 | −0.914 | −1.038 | −1.254 | −1.388 | −0.614 | −1.108 | −0.650 |
| P2RY10-Tango | 0.258 | −0.177 | −0.562 | −0.885 | −1.629 | −0.401 | −0.927 | −0.705 |
| P2RY11-Tango | −0.174 | −0.835 | −0.365 | −1.106 | −1.701 | −1.394 | −0.669 | −0.431 |
| P2RY12-Tango | −0.240 | −0.111 | −0.496 | −1.404 | −1.840 | −0.747 | −0.550 | −0.586 |
| P2RY13-Tango | 0.071 | −0.255 | −0.365 | −1.730 | −1.699 | −0.516 | −1.253 | −0.821 |
| P2RY14-Tango | 0.304 | −0.538 | −0.612 | −1.201 | −1.635 | −0.341 | −1.060 | −0.305 |
| P2RY2-Tango | −0.112 | −0.648 | −0.685 | −1.684 | −1.949 | −0.840 | −1.104 | −0.702 |
| P2RY4-Tango | −0.322 | −0.543 | −0.744 | −1.806 | −2.051 | −1.043 | −1.602 | −1.299 |
| P2RY6-Tango | 0.074 | −0.607 | −0.634 | −1.664 | −2.068 | −0.296 | −1.023 | −0.776 |
| P2RY8-Tango | −0.148 | −0.397 | −0.637 | −1.313 | −1.845 | −0.527 | −1.476 | −0.815 |
| PK1-Tango | 0.168 | −0.246 | −0.637 | −1.323 | −1.908 | −0.527 | −0.788 | −0.919 |
| PK2-Tango | −0.013 | 0.255 | −0.181 | −1.328 | −1.574 | −0.079 | −0.444 | −0.444 |
| PRRP-Tango | 0.100 | −0.334 | −0.202 | −1.525 | −1.978 | −0.286 | −0.442 | −0.323 |
| PTAFR-Tango | 0.029 | −0.840 | −0.902 | −0.986 | −1.366 | −0.258 | −0.586 | 0.079 |
| PTGDR-Tango | −0.133 | −0.580 | −0.822 | −1.355 | −1.777 | −0.646 | −0.674 | −0.958 |
| PTGER1-Tango | −0.021 | 0.293 | 0.762 | −0.755 | −1.296 | −0.085 | −0.613 | −0.756 |
| PTGER2-Tango | 0.356 | 0.471 | 0.898 | −1.303 | −1.389 | −0.074 | −0.564 | −0.328 |
| PTGER3-Tango | −0.010 | −0.409 | −0.125 | −1.164 | −1.462 | −0.057 | −0.825 | −0.226 |
| PTGER4-Tango | 0.026 | −0.343 | −0.051 | −1.074 | −1.699 | −0.038 | −0.796 | −0.499 |
| PTGFR-Tango | −0.136 | −0.580 | −0.032 | −1.795 | −2.596 | −0.592 | −1.942 | −0.615 |
| PTGIR-Tango | −0.027 | −0.411 | −1.394 | −1.215 | −1.412 | −1.156 | −1.291 | −0.493 |
| PTH1R-Tango | −0.436 | −0.401 | −0.457 | −1.095 | −2.021 | −0.094 | −1.147 | −0.867 |
| PTH2R-Tango | −0.425 | −0.678 | −0.696 | −0.921 | −1.248 | −0.049 | −1.130 | −0.201 |
| QRFP-Tango | −0.474 | −0.142 | −0.206 | −1.147 | −1.787 | −0.091 | −0.971 | −0.601 |
| RXFP1-Tango | −0.588 | −0.476 | −0.792 | −0.573 | −0.866 | −0.636 | −0.697 | −0.400 |
| RXFP2-Tango | −0.926 | −1.037 | −1.199 | −1.230 | −1.536 | −0.460 | −1.215 | −1.015 |
| RXFP3-Tango | −0.436 | −0.525 | −0.922 | −0.943 | −1.548 | −0.121 | −1.285 | −1.442 |
| RXFP4-Tango | −0.495 | −0.437 | −0.505 | −1.070 | −1.461 | −0.284 | −0.465 | −0.355 |
| S1PR1-Tango | −0.455 | −0.038 | 0.215 | −0.812 | −1.141 | −0.171 | 0.592 | 1.055 |
| S1PR2-Tango | −0.578 | −0.465 | −0.927 | −1.865 | −2.063 | −0.718 | 0.023 | 0.248 |
| S1PR3-Tango | −0.529 | −0.160 | 0.080 | −1.458 | −1.597 | 0.032 | −0.389 | −0.155 |
| S1PR4-Tango | −0.446 | −0.483 | −0.334 | −2.274 | −2.361 | −0.383 | −0.817 | −0.524 |
| S1PR5-Tango | −0.114 | 0.125 | −0.136 | −1.222 | −1.897 | 0.284 | −1.350 | −0.216 |
| SCTR-Tango | 0.132 | 0.620 | 1.201 | −1.684 | −1.808 | −0.224 | 0.154 | −0.723 |
| SSTR1-Tango | −0.690 | −0.068 | −0.068 | −1.867 | −2.063 | −0.490 | −1.252 | −0.545 |
| SSTR2-Tango | −0.729 | 0.322 | −0.333 | −1.075 | −1.403 | −0.228 | 0.140 | 0.000 |
| SSTR3-Tango | 0.000 | −0.697 | 0.243 | −1.029 | −1.305 | −0.021 | −0.718 | 0.082 |
| SSTR4-Tango | 0.287 | −0.468 | 0.127 | −1.688 | −1.442 | 0.428 | −1.148 | −0.392 |
| SSTR5-Tango | 1.129 | 0.660 | 0.399 | −0.773 | −0.985 | 1.286 | −0.268 | −0.284 |
| SUCNR1-Tango | 3.428 | 2.283 | 2.551 | 2.860 | 2.371 | 3.911 | 1.794 | 2.560 |
| TA1-Tango | −0.359 | −0.509 | −0.614 | −0.632 | −1.625 | 0.602 | −0.653 | −0.289 |
| TAAR2-Tango | −0.050 | −0.764 | −0.856 | −0.571 | −1.527 | −0.109 | −0.379 | −0.657 |
| TAAR5-Tango | 0.305 | −0.491 | −0.676 | −1.021 | −1.668 | −0.057 | −1.000 | −0.843 |
| TAAR6-Tango | −0.480 | −0.750 | −0.866 | −0.734 | −0.894 | 0.032 | −0.962 | −0.373 |
| TAAR8-Tango | −0.306 | −0.573 | −0.447 | −0.792 | −1.145 | 0.030 | −0.623 | −0.336 |
| TAAR9-Tango | −0.210 | −0.261 | −0.657 | −0.713 | −1.020 | −0.231 | −1.152 | −0.463 |
| TACR1-Tango | −0.197 | −0.666 | −0.471 | −0.841 | −1.058 | −0.068 | −0.790 | 0.021 |
| TACR2-Tango | −0.086 | −0.246 | −0.360 | −0.831 | −1.397 | 0.084 | −0.416 | −0.409 |
| TACR3-Tango | −0.407 | −0.764 | −0.723 | −1.293 | −1.867 | 0.139 | −0.577 | −0.238 |
| TBXA2R-Tango | −0.184 | −0.333 | −0.315 | −0.917 | −1.925 | −0.051 | −0.451 | −0.567 |
| TSHR-Tango | −0.745 | −1.244 | −1.178 | −1.271 | −1.865 | −0.428 | −1.503 | −0.845 |
| UTS2R-Tango | −0.054 | 0.176 | −0.503 | −1.025 | −1.816 | −0.101 | −0.858 | −0.629 |
| VIPR1-Tango | −0.060 | −0.550 | −0.266 | −1.823 | −1.948 | −0.242 | −1.677 | −0.842 |
| VIPR2-Tango | 1.222 | −0.305 | −0.405 | −1.361 | −1.604 | 0.300 | 0.270 | 1.372 |
| | (Enterobac- | (Enterobac- | (Enterobac- | (Enterobac- | (Enterobac- | (Enterobac- | (Enterobac- | (Enterobac- |
| PRESTO-Tango | teria)UC/ | teria)UC/ | teria)UC/ | teria)UC/ | teria)UC/ | teria)UC/ | teria)UC/ | teria)UC/ |
| with GMM | UC-6# | UC-7# | UC-8# | UC-9# | UC-10# | UC-11# | UC-12# | UC-13# |
| ADCYAP1R1- | −0.406 | −0.401 | −0.354 | 0.212 | −0.306 | 0.159 | 0.457 | 0.389 |
| Tango | | | | | | | | |
| ADORA1-Tango | −1.118 | −0.770 | −0.603 | 0.241 | −1.284 | −0.643 | −0.026 | −0.028 |
| ADORA2A-Tango | 0.021 | −0.458 | −0.154 | 0.434 | −0.651 | −0.331 | 0.089 | 0.327 |
| ADORA2B-Tango | −0.358 | −0.248 | −0.156 | −0.133 | −0.372 | −0.534 | −0.223 | 0.022 |
| ADORA3-Tango | −0.130 | 0.139 | 0.241 | 0.508 | −0.303 | 0.343 | −0.064 | 0.537 |
| ADRA1A-Tango | −0.188 | −0.006 | 0.209 | 0.155 | −0.409 | −0.451 | −0.284 | 0.404 |
| ADRA1B-Tango | −0.119 | −0.095 | 0.078 | 0.262 | −0.467 | 0.154 | −0.808 | −0.129 |
| ADRA1D-Tango | −0.178 | −0.082 | 0.019 | 0.104 | −0.467 | −0.425 | −0.224 | 0.411 |
| ADRA2A-Tango | −0.316 | −0.220 | −0.060 | −0.038 | −1.032 | −0.058 | 0.181 | 0.405 |
| ADRA2B-Tango | −1.089 | −0.916 | −0.887 | −0.360 | −1.423 | −1.044 | −0.501 | −0.906 |
| ADRA2C-Tango | −0.830 | −0.631 | −0.563 | 0.062 | −0.600 | −0.288 | 0.282 | 0.631 |
| ADRB1-Tango | −0.224 | −0.325 | 0.474 | −0.316 | 0.202 | 0.574 | 0.032 | 0.468 |
| ADRB2-Tango | −0.974 | −0.629 | −0.669 | −0.360 | −0.044 | −0.542 | 0.455 | 0.133 |
| ADRB3-Tango | −0.330 | −0.690 | −0.792 | 0.035 | 0.122 | −0.087 | −0.397 | −0.549 |
| AGTR1-Tango | −0.040 | −0.461 | −0.227 | 0.328 | −0.199 | −0.430 | 0.453 | 0.616 |
| AGTR2-Tango | −0.500 | 0.010 | −0.222 | 0.207 | −0.369 | 0.151 | 0.493 | 0.730 |
| APJ-Tango | −0.659 | −0.472 | −0.023 | −0.014 | −1.107 | −1.162 | 0.232 | −0.034 |
| AVPR1A-Tango | −0.615 | −0.389 | −0.092 | 0.021 | −0.189 | −0.962 | −0.395 | −0.127 |
| AVPR1B-Tango | −0.280 | −0.233 | −0.204 | 0.100 | −0.265 | −0.424 | −0.464 | −0.374 |
| AVPR2-Tango | −1.137 | −0.847 | −0.579 | 0.053 | −0.985 | −0.834 | 0.232 | 0.359 |
| BB3-Tango | −0.451 | −0.311 | −0.258 | 0.155 | −0.298 | −0.213 | −0.581 | −0.549 |
| BDKBR1-Tango | −0.081 | 0.236 | −0.210 | 0.150 | −0.106 | −0.672 | −0.559 | −0.613 |
| BDKBR2-Tango | 0.271 | 0.161 | 0.291 | 0.209 | 0.020 | 0.368 | 0.052 | 0.364 |
| C3AR1-Tango | −0.384 | −0.407 | −0.119 | 0.174 | −0.149 | −0.266 | −0.467 | −0.105 |
| C5A-Tango | 0.187 | 0.086 | 0.548 | 0.458 | −0.047 | −0.027 | −0.530 | 0.308 |
| CALCRb-Tango | −0.107 | 0.000 | 0.068 | 0.293 | −0.060 | −0.705 | −0.413 | −0.474 |
| CALCRL-Tango | −0.251 | −0.087 | 0.027 | 0.355 | −0.120 | −0.389 | −0.468 | −0.047 |
| CASR-Tango | −0.327 | −0.389 | −0.147 | 0.227 | 0.297 | −0.135 | −1.413 | −1.094 |
| CCKAR-Tango | 0.064 | 0.258 | 0.271 | 0.262 | −0.067 | 0.207 | −0.588 | 0.094 |
| CCKBR-Tango | −0.336 | −0.053 | −0.234 | −0.023 | −0.425 | −0.851 | −0.448 | −0.739 |
| CCR10-Tango | −0.628 | −0.693 | −0.363 | −0.315 | −0.369 | −0.632 | −0.384 | −0.299 |
| CCR2-Tango | −0.284 | 0.038 | −0.059 | −0.013 | −0.210 | −0.575 | −0.993 | −0.748 |
| CCR3-Tango | 0.026 | 0.451 | 0.073 | 0.364 | −0.810 | −0.237 | 0.266 | 0.604 |
| CCR4-Tango | −0.429 | 0.130 | −0.188 | 0.335 | −0.341 | −0.179 | −0.172 | 0.142 |
| CCR5-Tango | 0.203 | 0.366 | 0.479 | 0.131 | 0.297 | 0.203 | 0.371 | 0.457 |
| CCR6-Tango | 0.148 | 0.469 | 0.469 | 0.093 | 0.203 | 0.297 | 0.489 | 0.616 |
| CCR7-Tango | −0.470 | −0.328 | −0.101 | 0.175 | −0.221 | −0.160 | −0.094 | −0.330 |
| CCR8-Tango | −0.487 | −0.265 | −0.302 | −0.355 | −0.642 | −0.705 | 0.181 | 0.373 |
| CCRL2-Tango | −0.135 | 0.108 | 0.218 | 0.575 | −0.547 | −0.055 | 0.415 | 0.565 |
| CD97-Tango | −0.302 | −0.435 | −0.022 | 0.627 | −0.514 | −0.354 | −0.687 | −0.994 |
| CHRM1-Tango | −0.537 | −0.963 | −0.413 | −0.118 | −0.876 | −0.589 | 0.136 | 0.463 |
| CHRM2-Tango | −0.916 | −0.880 | −0.827 | 0.026 | −0.852 | −0.966 | −0.452 | −0.835 |
| CHRM3-Tango | −0.990 | −0.754 | −0.826 | 0.394 | −0.483 | −1.493 | 0.249 | 0.415 |
| CHRM4-Tango | −0.415 | −0.011 | −0.205 | 0.062 | 0.070 | −0.065 | −0.082 | 0.216 |
| CHRM5-Tango | −0.399 | −0.238 | −0.263 | 0.180 | 0.350 | 0.460 | −0.133 | 0.154 |
| CMKLR1-Tango | −0.220 | −0.212 | 0.000 | 0.159 | −0.535 | −0.493 | −0.228 | −0.170 |
| CNR1-Tango | −0.477 | −0.422 | −0.416 | 0.280 | −0.107 | −1.473 | −0.218 | −0.046 |
| CNR2-Tango | −0.288 | −0.201 | −0.183 | 0.311 | −0.678 | −0.704 | 0.055 | 0.601 |
| CRHR1-Tango | −0.064 | 0.455 | 0.037 | 0.225 | 0.057 | −0.336 | 0.525 | 0.000 |
| CRHR2-Tango | −0.702 | −0.348 | 0.088 | 0.040 | −0.195 | −0.353 | −0.345 | 1.160 |
| CX3C1-Tango | 0.119 | −0.286 | 0.055 | −0.045 | −0.186 | −0.604 | −0.445 | −0.245 |
| CXCR1-Tango | 0.371 | −0.040 | −0.089 | −0.160 | 0.504 | 0.327 | −0.129 | 1.017 |
| CXCR2-Tango | 0.670 | 1.019 | 0.660 | −0.052 | 0.625 | 0.867 | 0.859 | 1.344 |
| CXCR3-Tango | −0.289 | −0.077 | −0.485 | 0.104 | −0.394 | −0.441 | 0.404 | 0.850 |
| CXCR4-Tango | −0.020 | −0.146 | −0.052 | 0.604 | −0.017 | 0.158 | 0.661 | 1.264 |
| CXCR5-Tango | 0.139 | −0.143 | 0.066 | 0.375 | −0.130 | −0.196 | 0.048 | 0.610 |
| CXCR6-Tango | −0.705 | −0.665 | −0.120 | −0.413 | 0.500 | −0.445 | 1.415 | 1.595 |
| CXCR7-Tango | −0.823 | −0.740 | −0.565 | 0.057 | −0.720 | −0.828 | 0.564 | 0.584 |
| CYSLTR1-Tango | −0.073 | 0.134 | −0.023 | 0.345 | 0.019 | −0.037 | 0.575 | 0.951 |
| CYSLTR2-Tango | 0.021 | 0.096 | −0.473 | 0.031 | 0.211 | −0.405 | 0.313 | 0.960 |
| DRD1-Tango | −1.165 | −1.047 | −0.986 | −0.016 | 0.295 | −0.819 | 0.928 | 1.006 |
| DRD2-Tango | −0.391 | −0.640 | −1.126 | 0.299 | −0.628 | −1.011 | 0.084 | 0.318 |
| DRD3-Tango | −0.100 | −0.728 | −1.451 | 0.447 | 0.594 | −0.246 | 0.565 | 0.437 |
| DRD4-Tango | −0.792 | −0.642 | −0.520 | 0.787 | 0.234 | 0.234 | −0.433 | 0.455 |
| DRD5-Tango | −0.478 | −0.392 | −0.273 | −0.284 | −0.040 | −0.682 | 0.029 | 0.318 |
| EDNRA-Tango | −0.254 | −0.019 | −0.031 | 0.369 | −0.218 | −0.301 | −0.060 | 0.226 |
| EDNRB-Tango | −0.214 | −0.277 | −0.218 | 0.191 | −0.278 | −0.684 | −0.138 | −0.081 |
| ELTD1-Tango | −0.437 | −0.485 | −0.432 | 0.308 | −0.561 | −1.097 | −0.604 | −0.844 |
| F2R-Tango | −0.149 | −0.086 | −0.252 | 0.179 | −0.226 | −0.865 | −0.222 | −0.033 |
| F2RL1-Tango | −0.335 | −0.211 | −0.366 | 0.100 | 0.037 | −0.449 | −0.336 | −0.090 |
| F2RL2-Tango | −0.609 | −0.448 | −0.405 | 0.377 | 0.050 | −0.071 | −0.153 | −0.282 |
| F2RL3-Tango | −0.280 | −0.416 | −0.379 | 0.330 | −0.184 | −0.598 | 0.003 | −0.005 |
| FFA1-Tango | −0.505 | −0.206 | −0.230 | 0.670 | −0.036 | 0.028 | −0.029 | −0.117 |
| FFA2-Tango | −0.729 | −0.735 | −0.782 | 0.413 | −0.417 | −0.644 | −0.050 | 0.013 |
| FFA3-Tango | −0.893 | −0.585 | −0.379 | 0.248 | 0.297 | −0.257 | 0.020 | 0.351 |
| FPR1-Tango | −0.061 | −0.005 | 0.224 | 0.446 | 0.766 | 0.030 | 0.975 | 0.210 |
| FPR2-Tango | −0.264 | −0.120 | −0.109 | 0.096 | 0.677 | −1.000 | 0.313 | 0.104 |
| FPR3-Tango | −0.857 | −0.442 | −0.323 | 0.291 | −0.394 | −0.410 | 0.298 | 0.170 |
| FSHR-Tango | −0.650 | −0.432 | −0.267 | 0.443 | −0.196 | −0.444 | −0.601 | −0.480 |
| GABBR1-Tango | −0.097 | 0.038 | −0.369 | −0.062 | 0.026 | −0.572 | 0.123 | −0.260 |
| GAL1-Tango | −0.940 | −0.330 | −0.940 | −0.361 | −1.117 | −1.296 | −0.470 | −0.698 |
| GAL2-Tango | −0.124 | 0.267 | −0.079 | 0.515 | −1.094 | −0.982 | 0.791 | 1.322 |
| GAL3-Tango | −0.444 | −0.415 | −0.263 | −0.198 | −0.501 | −0.968 | −0.962 | 0.026 |
| GCGR-Tango | −0.366 | −0.240 | −0.289 | 0.194 | −0.285 | −0.876 | −0.301 | 0.125 |
| GHRHR-Tango | −0.467 | −0.115 | −0.284 | 0.241 | −0.131 | −0.942 | −0.210 | −0.079 |
| GHSR-Tango | −0.977 | −0.721 | −0.458 | 0.429 | −0.650 | −0.622 | 0.169 | 0.532 |
| GIPR-Tango | −0.677 | −0.544 | −0.299 | −0.054 | −0.342 | −1.245 | 0.000 | 0.000 |
| GLP1R-Tango | −0.753 | −0.303 | −0.354 | 0.276 | −0.970 | −1.348 | −0.571 | −0.348 |
| GLP2R-Tango | −0.178 | 0.309 | 0.309 | 0.005 | −0.422 | −0.579 | 0.261 | 0.614 |
| GNRHR-Tango | −0.592 | −0.235 | −0.393 | −0.293 | −0.542 | −1.044 | 0.000 | −0.060 |
| GPBA-Tango | −0.990 | −0.527 | −0.379 | 0.371 | −0.510 | −0.220 | 0.945 | 0.803 |
| GPER-Tango | −0.034 | 0.165 | 0.229 | 0.343 | −0.057 | −0.468 | −0.312 | 0.330 |
| GPR1-Tango | −0.543 | −0.366 | −0.169 | 0.141 | −0.343 | −0.265 | 0.050 | −0.089 |
| GPR101-Tango | −0.058 | 0.036 | −0.082 | 0.261 | 0.059 | 0.065 | 0.359 | 0.291 |
| GPR110-Tango | −0.312 | −0.217 | −0.042 | −0.018 | −0.432 | −0.408 | −0.538 | −0.460 |
| GPR111-Tango | −0.195 | −0.120 | −0.048 | −0.201 | −0.135 | −0.579 | −0.377 | −0.467 |
| GPR113-Tango | −0.963 | −0.582 | −0.510 | 0.089 | −0.331 | −0.820 | −0.441 | −0.153 |
| GPR114-Tango | −0.281 | −0.322 | −0.400 | −0.204 | −0.274 | −0.938 | −0.053 | −0.421 |
| GPR115-Tango | −0.227 | −0.445 | −0.497 | 0.050 | −0.519 | −0.272 | −0.631 | −0.493 |
| GPR116-Tango | −0.221 | −0.176 | −0.237 | 0.101 | −0.835 | −0.778 | −0.609 | −0.771 |
| GPR119-Tango | −0.402 | −0.101 | −0.268 | 0.094 | −0.509 | −0.687 | 0.000 | 0.188 |
| GPR12-Tango | −0.335 | 0.084 | −0.089 | 0.376 | 0.149 | −0.098 | 0.349 | 0.518 |
| GPR120-Tango | −0.109 | 0.072 | −0.296 | 0.015 | −0.477 | −0.500 | −0.355 | −0.105 |
| GPR123-Tango | −0.319 | −0.115 | 0.231 | 0.291 | −0.113 | 0.015 | 0.202 | 0.778 |
| GPR124-Tango | −0.292 | −0.664 | −0.623 | 0.099 | −0.130 | −0.423 | −0.335 | 0.133 |
| GPR125-Tango | −0.500 | −0.562 | −0.300 | −0.209 | 0.256 | 0.076 | −0.147 | 0.109 |
| GPR126-Tango | −0.748 | −0.489 | −0.689 | −0.088 | −0.431 | −0.924 | −0.548 | −0.586 |
| GPR132-Tango | −1.342 | −0.897 | −0.878 | 0.397 | −1.119 | −1.261 | −0.601 | −0.372 |
| GPR133-Tango | −0.644 | −0.629 | −0.634 | 0.281 | −0.307 | −0.436 | −0.527 | −0.181 |
| GPR135-Tango | −0.473 | −0.562 | −0.409 | −0.185 | −0.019 | −0.727 | −0.040 | 0.100 |
| GPR141-Tango | −0.023 | −0.301 | −0.414 | 0.635 | −0.093 | −0.422 | 0.286 | 0.314 |
| GPR142-Tango | −0.492 | −0.593 | −0.501 | 0.125 | −0.657 | −0.785 | 0.068 | 0.575 |
| GPR143-Tango | −0.736 | −0.579 | −0.390 | 0.400 | −0.694 | −0.763 | −0.605 | −0.651 |
| GPR144-Tango | −0.927 | −0.358 | −0.449 | −0.029 | −0.846 | −1.968 | −0.840 | −0.523 |
| GPR146-Tango | 0.154 | −0.395 | 0.745 | 0.531 | 0.766 | −0.368 | −0.104 | 0.000 |
| GPR148-Tango | 0.341 | 0.968 | 0.968 | −0.066 | 0.282 | 0.000 | 1.026 | −0.284 |
| GPR149-Tango | −0.260 | 0.038 | −0.135 | 0.144 | 0.506 | 0.126 | −0.317 | 0.816 |
| GPR15-Tango | −0.243 | −0.170 | −0.132 | 0.361 | −0.358 | −0.554 | 0.313 | 0.718 |
| GPR150-Tango | 0.437 | 0.102 | 0.357 | −0.160 | 0.000 | −0.463 | 0.112 | 0.376 |
| GPR151-Tango | −0.414 | −0.165 | −0.526 | 0.544 | 0.195 | 0.054 | −0.329 | −0.302 |
| GPR152-Tango | −0.612 | −0.392 | −0.574 | −0.077 | −0.384 | −0.908 | −0.607 | −0.398 |
| GPR153-Tango | −0.370 | −0.434 | −0.446 | 0.087 | 0.063 | 0.147 | 0.016 | 0.301 |
| GPR156-Tango | −0.570 | −0.536 | −0.678 | −0.232 | −0.793 | −1.106 | −0.002 | 0.023 |
| GPR157-Tango | −0.423 | −0.528 | −0.331 | 0.215 | −0.169 | −0.956 | 0.058 | 0.150 |
| GPR158-Tango | 0.029 | −0.114 | −0.114 | 0.065 | −0.178 | −0.934 | −0.672 | −0.526 |
| GPR160-Tango | −0.315 | −0.390 | −0.315 | 0.247 | 0.041 | −0.219 | −0.158 | 0.218 |
| GPR161-Tango | −0.356 | −0.332 | −0.237 | 0.176 | 0.441 | 0.115 | 0.227 | 0.618 |
| GPR162-Tango | −0.601 | −0.386 | −0.714 | −0.092 | −0.098 | −0.431 | −0.287 | 0.203 |
| GPR17-Tango | −0.854 | −0.774 | −0.750 | 0.463 | 0.363 | 0.393 | 0.446 | 0.603 |
| GPR171-Tango | −0.631 | −0.503 | −0.592 | 0.190 | −0.341 | −0.547 | 0.200 | 0.411 |
| GPR173-Tango | −0.614 | −0.371 | −0.494 | −0.174 | 0.326 | −0.532 | 0.163 | 0.186 |
| GPR174-Tango | −0.043 | −0.306 | −0.360 | 0.526 | −0.211 | −0.495 | 0.265 | 0.688 |
| GPR18-Tango | −0.469 | −0.196 | −0.405 | 0.316 | −0.218 | −0.672 | 0.278 | 0.404 |
| GPR182-Tango | −0.692 | −0.671 | −0.716 | 0.602 | 0.107 | 0.075 | 0.106 | 0.241 |
| GPR183-Tango | −0.344 | −0.279 | −0.661 | 0.507 | −0.165 | −0.591 | 0.204 | 0.000 |
| GPR19-Tango | −0.774 | −0.823 | −0.749 | 0.652 | 0.132 | 0.079 | −0.488 | −0.351 |
| GPR20-Tango | −0.048 | 0.423 | 0.000 | 1.322 | 1.036 | −0.368 | 0.104 | 0.350 |
| GPR21-Tango | 0.105 | 0.022 | −0.344 | 0.629 | 0.033 | −0.613 | 0.000 | 0.118 |
| GPR22-Tango | 0.016 | 0.236 | 0.125 | 0.293 | 0.082 | −0.118 | 0.642 | 0.660 |
| GPR25-Tango | −0.299 | 0.226 | 0.132 | 0.070 | 0.077 | −0.728 | 0.165 | 0.301 |
| GPR26-Tango | −0.431 | −0.259 | −0.243 | 0.332 | 0.627 | 0.031 | 0.054 | 0.216 |
| GPR27-Tango | 0.221 | 0.023 | −0.428 | 0.214 | 0.214 | −0.534 | 0.803 | 0.595 |
| GPR3-Tango | −0.762 | −0.852 | −0.812 | 0.559 | −0.179 | −0.185 | 0.620 | 0.668 |
| GPR31-Tango | −0.842 | −1.057 | −0.998 | 0.750 | −0.403 | −0.218 | 0.154 | 0.503 |
| GPR32-Tango | −0.019 | −0.307 | −0.162 | 0.428 | −0.379 | −0.799 | 0.293 | 0.171 |
| GPR34-Tango | −0.549 | −0.288 | −0.350 | 0.288 | −0.669 | −0.650 | 0.172 | 0.372 |
| GPR35-Tango | −1.176 | −0.564 | −0.596 | 0.396 | −0.783 | −0.480 | 0.225 | 0.168 |
| GPR37-Tango | −0.526 | −0.068 | 0.119 | 0.482 | −0.274 | 0.173 | 0.539 | 0.839 |
| GPR37L1-Tango | −0.259 | −0.092 | −0.087 | 0.106 | 0.205 | 0.063 | −0.329 | −0.256 |
| GPR39-Tango | −0.259 | −0.129 | −0.185 | −0.035 | −0.304 | −0.705 | −0.446 | −0.570 |
| GPR4-Tango | 0.100 | 0.065 | 0.083 | 0.372 | −0.135 | 0.138 | 0.389 | 0.606 |
| GPR44-Tango | −0.385 | −0.345 | −0.350 | 0.023 | −0.090 | −0.566 | 0.050 | −0.057 |
| GPR45-Tango | −0.409 | −0.515 | −0.655 | 0.108 | −0.411 | −0.293 | 0.385 | 0.145 |
| GPR50-Tango | 0.516 | 0.436 | 0.206 | 0.475 | 0.160 | 0.365 | 0.453 | 1.368 |
| GPR52-Tango | 0.216 | −0.020 | 0.233 | −0.211 | 0.108 | 0.175 | 0.898 | 0.502 |
| GPR55-Tango | −0.410 | −0.164 | 0.009 | 0.260 | −0.560 | −0.117 | 0.490 | 0.358 |
| GPR56-Tango | −0.366 | 0.039 | −0.026 | 0.352 | −0.432 | −0.192 | −0.369 | −0.006 |
| GPR6-Tango | −0.052 | −0.058 | 0.357 | 0.540 | 0.037 | 0.444 | 0.626 | 0.915 |
| GPR61-Tango | −0.357 | −0.153 | −0.185 | 0.250 | 0.025 | 0.160 | 0.397 | 0.381 |
| GPR62-Tango | −0.324 | 0.013 | 0.144 | 0.303 | −0.269 | −0.112 | −0.379 | 0.140 |
| GPR63-Tango | −0.246 | 0.188 | −0.213 | −0.252 | −0.156 | −0.123 | −0.486 | −0.224 |
| GPR64-Tango | −0.124 | −0.091 | −0.177 | −0.293 | −0.031 | −0.247 | −0.143 | 0.196 |
| GPR65-Tango | 0.325 | 0.483 | 0.198 | −0.035 | −0.044 | 0.153 | 0.048 | 0.251 |
| GPR68-Tango | −0.111 | −0.121 | −0.139 | −0.055 | −0.403 | −0.545 | −0.181 | 0.149 |
| GPR75-Tango | 0.179 | 0.510 | 0.619 | 0.219 | 0.069 | −0.003 | 0.586 | 1.155 |
| GPR77-Tango | −0.561 | −0.245 | −0.221 | −0.058 | −0.495 | −0.547 | −0.052 | 0.315 |
| GPR78-Tango | −0.285 | 0.189 | 0.224 | 0.208 | 0.318 | −0.088 | −0.008 | 0.378 |
| GPR82-Tango | −1.345 | −0.674 | −0.334 | −0.066 | −0.491 | −0.331 | 0.098 | 1.013 |
| GPR83-Tango | −0.115 | −0.004 | 0.181 | 0.239 | −0.326 | −0.423 | 0.009 | 0.428 |
| GPR84-Tango | −0.037 | 0.157 | 0.248 | 0.215 | −0.633 | −0.459 | 0.369 | 0.831 |
| GPR85-Tango | 0.210 | 0.063 | 0.179 | 0.110 | −0.605 | −0.513 | 0.183 | 0.454 |
| GPR87-Tango | −0.138 | 0.404 | 0.472 | 0.230 | −0.218 | 0.277 | 0.612 | 1.513 |
| GPR88-Tango | −0.029 | 0.178 | −0.022 | 0.090 | −0.370 | −0.417 | 0.524 | 0.631 |
| GPR97-Tango | −0.286 | −0.025 | −0.025 | 0.335 | −0.347 | −0.655 | 0.272 | 0.305 |
| GPRC5A-Tango | −0.538 | −0.157 | −0.118 | 0.470 | −0.417 | −0.188 | 0.251 | 0.480 |
| GPRC5B-Tango | −0.339 | −0.085 | 0.131 | 0.516 | −0.513 | −0.239 | 0.054 | 0.645 |
| GPRC5C-Tango | 0.240 | 0.596 | 0.584 | 0.523 | −0.317 | −0.227 | 0.597 | 0.553 |
| GPRC5D-Tango | −0.054 | 0.007 | 0.028 | −0.040 | −0.077 | −0.667 | −0.209 | −0.110 |
| GPRC6A-Tango | 0.187 | 0.356 | 0.637 | −0.069 | −0.579 | −0.813 | −0.112 | 0.014 |
| GRM1-Tango | 0.070 | −0.038 | 0.252 | −0.202 | 0.036 | −0.896 | −0.071 | −0.450 |
| GRM2-Tango | 0.000 | −0.052 | 0.043 | −0.125 | −0.386 | −1.006 | −0.226 | −0.116 |
| GRM4-Tango | 0.065 | 0.307 | 0.408 | 0.598 | −0.115 | 0.176 | −0.372 | 0.125 |
| GRM5-Tango | 0.251 | 0.259 | 0.937 | −0.349 | 0.198 | −0.602 | 0.061 | −0.107 |
| GRM6-Tango | −0.339 | −0.124 | −0.035 | −0.478 | 0.218 | −0.556 | −0.159 | 0.125 |
| GRM7-Tango | 0.367 | 0.312 | 0.230 | −0.476 | −0.775 | −1.329 | −0.548 | −0.443 |
| GRM8-Tango | −0.359 | −0.180 | −0.171 | −0.154 | −0.335 | −0.865 | −0.117 | −0.650 |
| GRPR-Tango | −0.075 | 0.226 | 0.817 | 0.313 | −0.232 | −0.614 | 0.745 | 1.136 |
| HCA1-Tango | −0.111 | −0.023 | −0.055 | 0.211 | −0.122 | −0.457 | 0.202 | 0.550 |
| HCA2-Tango | 0.247 | 0.671 | 0.752 | 0.925 | −0.553 | −0.400 | 1.186 | 0.741 |
| HCA3-Tango | −0.793 | −0.381 | −0.165 | 0.597 | −0.643 | −1.040 | 0.291 | 0.437 |
| HCTR1-Tango | −0.376 | −0.216 | −0.136 | 0.840 | 0.577 | −0.248 | 0.719 | 0.322 |
| HCTR2-Tango | −1.350 | −0.350 | −0.154 | −0.248 | 0.545 | 0.282 | −0.387 | −0.133 |
| HRH1-Tango | −0.526 | −0.255 | −0.467 | 0.201 | −0.138 | 0.307 | 0.708 | 0.845 |
| HRH2-Tango | −0.087 | −0.365 | 1.440 | 0.145 | −0.056 | −0.081 | 0.276 | 0.343 |
| HRH3-Tango | 0.251 | 0.452 | 0.820 | −0.568 | 0.162 | 0.338 | 0.817 | 1.741 |
| HRH4-Tango | 0.650 | 1.103 | 0.871 | 0.081 | 0.625 | 0.436 | 0.511 | 1.860 |
| HTR1A-Tango | 0.198 | 0.406 | 0.221 | 0.278 | 0.041 | 0.556 | 0.395 | 1.002 |
| HTR1B-Tango | 0.022 | 0.243 | 0.056 | 0.398 | −0.201 | 0.526 | 0.731 | 1.170 |
| HTR1D-Tango | −0.381 | −0.088 | −0.106 | 0.249 | −0.474 | 0.263 | 0.472 | 0.824 |
| HTR1E-Tango | −0.132 | 0.111 | 0.138 | 0.396 | −0.006 | 0.797 | 0.397 | 1.073 |
| HTR1F-Tango | −1.334 | −0.763 | −0.212 | 0.691 | −0.365 | 0.344 | 0.498 | 0.741 |
| HTR2A-Tango | −0.183 | 0.052 | 0.241 | −0.118 | −0.248 | −0.396 | 0.443 | 0.419 |
| HTR2B-Tango | −0.149 | 0.127 | 0.269 | 0.336 | −0.243 | −0.420 | 0.336 | 0.359 |
| HTR2C-Tango | −0.262 | −0.220 | −0.266 | −0.087 | −0.065 | −0.681 | −0.402 | −0.208 |
| HTR4-Tango | 0.100 | −0.071 | 0.090 | 0.286 | 0.306 | 0.194 | 0.579 | 0.513 |
| HTR5-Tango | −1.302 | −0.684 | −0.146 | 0.528 | 0.108 | 0.121 | 0.991 | 1.311 |
| HTR6-Tango | 0.120 | 0.187 | 0.413 | 0.399 | 0.053 | −0.149 | 0.159 | 0.495 |
| HTR7-Tango | 0.312 | 0.625 | 0.694 | 0.400 | 0.367 | 0.439 | 0.304 | 0.604 |
| KISSPEPTIN-R- | −0.092 | 0.127 | −0.092 | −0.642 | 0.326 | −0.631 | 0.122 | −0.065 |
| Tango | | | | | | | | |
| LHCGR-Tango | −0.457 | −0.357 | −0.006 | 0.303 | −0.489 | −0.284 | −0.379 | −0.038 |
| LPAR1-Tango | −0.917 | −0.262 | 0.078 | 0.410 | −0.140 | −0.230 | 0.306 | 0.602 |
| LPAR2-Tango | −1.047 | −0.624 | −0.223 | 0.035 | −0.312 | −0.753 | −0.120 | −0.015 |
| LPAR4-Tango | −1.946 | −1.012 | −0.545 | 0.215 | −0.173 | −0.296 | 0.100 | 0.360 |
| LPAR5-Tango | −0.656 | −0.486 | −0.392 | −0.030 | −0.152 | −0.816 | −0.048 | −0.012 |
| LPAR6-Tango | −0.386 | −0.086 | 0.049 | 0.428 | 0.460 | 0.610 | 0.720 | 0.992 |
| LTB4R-Tango | −0.468 | −0.089 | −0.295 | 0.036 | 0.246 | −0.097 | 0.257 | 0.635 |
| LTB4R2B-Tango | 0.182 | 0.222 | 0.178 | 0.360 | −0.132 | 0.030 | 0.244 | 0.328 |
| MAS1-Tango | −0.191 | 0.246 | 0.108 | −0.037 | −0.242 | −0.008 | 0.089 | 0.162 |
| MAS1L-Tango | 0.001 | 0.161 | 0.311 | −0.123 | 0.050 | −0.009 | −0.273 | −0.544 |
| MC1R-Tango | −0.162 | 0.252 | 0.047 | −0.010 | −0.025 | −0.568 | 0.102 | 0.065 |
| MC2R-Tango | 0.123 | −0.200 | 0.096 | 0.242 | 0.171 | −0.163 | 0.520 | 0.628 |
| MC3R-Tango | −0.100 | −0.117 | 0.155 | 0.078 | −0.234 | −0.098 | −0.122 | 0.049 |
| MC4R-Tango | 0.000 | 0.405 | 0.381 | 0.681 | −0.049 | −0.429 | 0.705 | 0.603 |
| MC5R-Tango | −0.130 | 0.085 | 0.442 | 0.509 | 0.273 | 0.031 | 0.390 | 0.435 |
| MCHR1-Tango | −0.709 | −0.691 | −0.670 | −1.153 | 0.153 | −0.124 | −0.441 | −0.094 |
| MCHR2-Tango | −0.209 | −0.518 | 0.157 | −0.246 | 0.235 | −0.178 | 0.026 | −0.418 |
| MLNR-Tango | −0.310 | −0.023 | −0.310 | −0.067 | −0.215 | −0.252 | 0.321 | 0.117 |
| MRGPRD-Tango | −0.471 | −0.170 | −0.410 | 0.068 | −0.169 | 0.071 | 0.033 | 0.244 |
| MRGPRE-Tango | −0.213 | −0.106 | −0.332 | −0.169 | −0.040 | −0.159 | 0.325 | 0.331 |
| MRGPRF-Tango | 0.035 | 0.327 | 0.295 | 0.312 | −0.132 | −0.012 | 0.319 | 0.412 |
| MRGPRG-Tango | 0.183 | 0.058 | 0.038 | −0.483 | 0.046 | 0.238 | −0.271 | −0.203 |
| MRGPRX1-Tango | −0.459 | −0.527 | −0.599 | −0.555 | 0.215 | −0.046 | −0.100 | 0.229 |
| MRGPRX2-Tango | −0.518 | −0.205 | 0.242 | −0.118 | −0.012 | −0.156 | 0.355 | 0.480 |
| MRGPRX3-Tango | −0.366 | −0.386 | 0.165 | −0.167 | −0.078 | −1.367 | 0.364 | −0.079 |
| MRGPRX4-Tango | −1.078 | −0.797 | −0.588 | −0.459 | −1.064 | −1.628 | −0.149 | −0.053 |
| MTNR1A-Tango | −0.497 | −0.014 | −0.023 | 0.171 | −0.714 | −0.483 | 0.173 | 0.748 |
| MTNR1B-Tango | −0.324 | 0.242 | −0.172 | 0.214 | −0.261 | −0.532 | 0.612 | 0.733 |
| NMBR-Tango | 0.141 | 0.074 | 0.455 | 0.134 | −0.291 | −0.257 | 0.284 | 0.733 |
| NMUR1-Tango | −0.418 | 0.116 | 0.186 | 0.318 | −0.930 | −1.130 | −0.650 | −0.087 |
| NMUR2-Tango | −0.780 | 0.357 | −0.145 | 1.170 | −0.445 | −0.992 | 0.894 | 1.085 |
| NPBW1-Tango | −0.543 | −0.848 | −0.750 | 0.564 | −0.127 | −0.196 | 0.313 | −0.199 |
| NPBW2-Tango | −0.592 | −0.249 | −0.498 | 0.640 | −0.733 | −0.011 | −0.570 | −0.429 |
| NPFF1-Tango | −0.052 | 0.074 | −0.070 | −1.074 | 0.033 | −0.665 | −0.608 | −0.293 |
| NPFF2-Tango | −0.358 | −0.194 | 0.124 | 0.062 | −0.262 | −0.480 | −0.041 | 0.673 |
| NPS-Tango | −0.429 | −0.311 | −0.202 | −0.426 | −0.292 | −0.149 | −0.215 | −0.087 |
| NPY1R-Tango | 0.093 | 0.026 | −0.130 | −0.031 | −0.087 | −0.236 | −0.381 | −0.326 |
| NPY2R-Tango | 0.262 | −0.178 | −0.055 | 0.155 | −0.449 | −0.088 | −0.171 | 0.265 |
| NPY4R-Tango | −0.423 | −0.486 | −0.177 | −0.017 | −0.435 | −0.720 | 0.379 | 0.360 |
| NPY5R-Tango | −0.047 | 0.012 | 0.208 | 0.065 | −0.778 | −0.790 | 0.063 | 0.197 |
| NTSR1-Tango | −0.298 | 0.029 | 0.085 | 0.401 | −1.176 | −0.789 | 0.323 | 0.263 |
| NTSR2-Tango | 0.064 | 0.413 | 0.265 | 0.396 | −0.492 | −0.863 | 0.137 | 0.401 |
| OPN3-Tango | 0.103 | 0.000 | −0.152 | 0.153 | −0.055 | 0.000 | 0.000 | −0.347 |
| OPN5-Tango | −0.695 | −0.262 | −0.281 | 0.477 | −0.865 | −0.871 | 0.122 | 0.321 |
| OPRD1-Tango | −0.521 | −0.390 | −0.616 | 0.299 | −0.636 | −1.390 | −0.219 | 0.330 |
| OPRK1-Tango | 0.514 | 0.532 | 0.356 | 0.437 | −0.151 | 0.082 | 0.402 | 0.560 |
| OPRL1-Tango | 0.026 | 0.243 | −0.091 | −0.336 | −0.202 | −0.563 | 0.846 | 0.505 |
| OPRM1-Tango | 0.299 | 0.012 | 0.610 | 0.299 | −0.284 | −0.365 | −0.313 | −0.291 |
| OXER1-Tango | 0.255 | 0.199 | 0.355 | 0.261 | −0.595 | 0.241 | −0.324 | −0.386 |
| OXGR1-Tango | −1.044 | −0.437 | −0.165 | 0.194 | −0.935 | −1.101 | 0.203 | 0.298 |
| OXTR-Tango | 0.074 | −0.160 | 0.027 | 0.157 | −0.094 | −1.110 | 0.162 | 0.162 |
| P2RY1-Tango | −0.480 | 0.099 | −0.090 | 0.421 | −1.167 | −0.780 | 0.019 | 0.432 |
| P2RY10-Tango | −0.121 | 0.304 | 0.149 | −0.222 | −0.619 | −1.031 | 0.224 | 0.069 |
| P2RY11-Tango | −0.179 | −0.248 | −0.050 | 0.287 | −0.645 | −1.214 | −0.260 | 0.019 |
| P2RY12-Tango | −0.230 | −0.081 | 0.069 | 0.494 | −1.236 | −0.937 | 0.401 | 0.468 |
| P2RY13-Tango | −0.770 | −0.433 | −0.664 | 0.293 | −0.451 | −0.261 | 0.436 | 0.526 |
| P2RY14-Tango | −0.517 | −0.755 | −0.729 | −0.059 | −0.449 | −0.067 | 0.244 | −0.035 |
| P2RY2-Tango | −0.660 | −0.387 | −0.750 | 0.187 | −0.777 | −0.842 | −0.002 | −0.207 |
| P2RY4-Tango | −0.975 | −0.965 | −0.920 | 0.326 | −0.631 | −0.820 | 0.190 | 0.318 |
| P2RY6-Tango | −0.719 | −0.881 | −0.708 | −0.080 | −0.728 | −0.648 | 0.333 | 0.625 |
| P2RY8-Tango | −0.553 | −0.745 | −0.471 | −0.306 | −0.434 | −0.581 | 0.012 | 0.460 |
| PK1-Tango | −0.500 | −0.635 | −0.384 | −0.255 | 0.087 | −0.373 | −0.128 | 0.118 |
| PK2-Tango | −0.128 | −0.330 | 0.118 | 0.263 | 0.000 | −0.626 | 0.125 | 0.125 |
| PRRP-Tango | −0.375 | −0.444 | −0.395 | 0.301 | −0.727 | −0.070 | 0.848 | 0.379 |
| PTAFR-Tango | −0.175 | −0.177 | −0.115 | 0.364 | −0.685 | −0.020 | 0.715 | 0.823 |
| PTGDR-Tango | −0.008 | −0.080 | −0.222 | 0.076 | −0.532 | −0.792 | 0.033 | 0.164 |
| PTGER1-Tango | 0.401 | 0.413 | 0.082 | 0.553 | −0.273 | −0.229 | 0.191 | 0.085 |
| PTGER2-Tango | 0.165 | 0.296 | −0.156 | 0.494 | −0.798 | −0.703 | 0.822 | 0.792 |
| PTGER3-Tango | −0.099 | 0.147 | 0.219 | 0.453 | −0.553 | −0.367 | 0.680 | 0.748 |
| PTGER4-Tango | 0.386 | 0.780 | 0.788 | 0.494 | 0.372 | 0.431 | 0.648 | 0.615 |
| PTGFR-Tango | −0.054 | −0.258 | −0.143 | 0.848 | −0.106 | −0.200 | 0.523 | 0.310 |
| PTGIR-Tango | −0.785 | −0.073 | −0.550 | 0.288 | −0.372 | −1.250 | 0.623 | 0.463 |
| PTH1R-Tango | −0.274 | −0.010 | −0.010 | 0.038 | −0.521 | −0.731 | 0.177 | 0.570 |
| PTH2R-Tango | −0.523 | −0.603 | −0.561 | 0.087 | −0.533 | −0.928 | 0.265 | 0.327 |
| QRFP-Tango | −0.039 | −0.147 | −0.155 | 0.266 | −0.817 | −1.152 | 0.053 | 0.196 |
| RXFP1-Tango | −0.205 | −0.209 | −0.320 | 0.102 | −0.310 | −1.234 | −0.706 | −0.817 |
| RXFP2-Tango | −1.088 | −0.893 | −1.081 | 0.056 | −0.782 | −0.904 | −0.141 | −0.086 |
| RXFP3-Tango | −0.624 | −0.262 | −0.702 | −0.093 | −0.425 | −1.194 | −0.020 | −0.139 |
| RXFP4-Tango | −0.362 | −0.386 | −0.666 | 0.350 | −0.645 | −0.645 | 0.208 | 0.405 |
| S1PR1-Tango | −0.070 | 0.020 | 0.240 | 0.589 | −0.663 | −0.235 | 0.038 | 0.218 |
| S1PR2-Tango | −0.655 | −0.118 | −0.264 | 0.503 | −0.515 | −0.185 | −0.019 | 0.258 |
| S1PR3-Tango | 0.375 | 0.478 | 0.527 | 0.610 | −0.684 | −0.543 | 0.230 | 0.537 |
| S1PR4-Tango | −1.248 | −0.733 | −0.488 | 0.180 | −0.918 | −0.737 | 0.398 | 0.553 |
| S1PR5-Tango | −0.453 | −0.713 | −0.199 | 0.000 | −0.585 | −0.415 | 0.376 | 0.376 |
| SCTR-Tango | −0.064 | −0.246 | 0.150 | 0.535 | −0.100 | 0.109 | 1.212 | 1.243 |
| SSTR1-Tango | 0.143 | 0.143 | 0.372 | 0.500 | −0.170 | −0.082 | 0.596 | 0.918 |
| SSTR2-Tango | 0.246 | 0.076 | 0.628 | 0.340 | −0.244 | 0.318 | −0.661 | 0.000 |
| SSTR3-Tango | 0.377 | 0.357 | 0.077 | 0.645 | 0.501 | 0.376 | 0.940 | 1.250 |
| SSTR4-Tango | −0.021 | 0.114 | 0.255 | −0.064 | 0.206 | 0.545 | 0.303 | 0.848 |
| SSTR5-Tango | 0.562 | 0.543 | 0.460 | 0.640 | 1.000 | 1.918 | 0.742 | 0.857 |
| SUCNR1-Tango | 2.594 | 2.560 | 2.697 | −0.075 | 3.681 | 4.105 | 2.733 | 3.230 |
| TA1-Tango | −0.090 | −0.265 | −0.135 | 0.313 | 0.047 | 0.273 | 0.603 | 0.852 |
| TAAR2-Tango | 0.057 | 0.014 | 0.165 | −0.160 | −0.422 | −0.415 | 0.070 | 0.520 |
| TAAR5-Tango | −0.241 | −0.271 | −0.438 | −0.860 | −0.390 | −0.526 | −0.157 | −0.157 |
| TAAR6-Tango | −0.269 | −0.258 | −0.334 | −0.484 | −0.073 | −0.037 | −0.356 | −0.316 |
| TAAR8-Tango | −0.625 | 0.191 | 0.135 | −0.721 | −0.225 | −0.213 | −0.238 | −0.365 |
| TAAR9-Tango | −0.376 | −0.196 | −0.380 | −0.715 | −0.030 | −0.311 | −0.357 | −0.426 |
| TACR1-Tango | −0.255 | −0.218 | −0.417 | −0.412 | −0.261 | 0.158 | 0.457 | 0.370 |
| TACR2-Tango | −0.119 | −0.175 | −0.206 | −0.299 | −0.318 | −0.252 | 0.015 | 0.330 |
| TACR3-Tango | −0.086 | 0.047 | 0.119 | −0.043 | −0.675 | −0.484 | 0.479 | 0.514 |
| TBXA2R-Tango | −0.243 | −0.220 | −0.419 | −0.192 | −0.131 | −0.176 | 0.212 | −0.100 |
| TSHR-Tango | −0.403 | −0.395 | −0.295 | −0.167 | −0.295 | −0.142 | −0.662 | −0.662 |
| UTS2R-Tango | −0.055 | 0.190 | −0.343 | −0.116 | 0.260 | −0.144 | −0.490 | −0.299 |
| VIPR1-Tango | −0.156 | −0.090 | 0.053 | −1.117 | −0.331 | −0.023 | −0.700 | −0.700 |
| VIPR2-Tango | 0.348 | 0.221 | 0.401 | 0.030 | 0.545 | 1.250 | 0.718 | 1.196 |

| | (Enterobac- | (Enterobac- | (Enterobac- | (Enterobac- | (Enterobac- | (Enterobac- | (Enterobac- |
|---|---|---|---|---|---|---|---|
| PRESTO-Tango | teria)UC/ | teria)UC/ | teria)UC/ | teria)UC/ | teria)UC/ | teria)UC/ | teria)UC/ |
| with GMM | UC-14# | UC-15# | UC-16# | UC-17# | UC-18# | UC-19# | UC-20# |
| ADCYAP1R1- | 0.475 | −0.223 | −0.137 | 0.864 | 0.380 | 0.245 | 0.210 |
| Tango | | | | | | | |
| ADORA1-Tango | −0.981 | −0.212 | −1.163 | 0.945 | 0.035 | −0.363 | −0.545 |
| ADORA2A-Tango | −0.457 | −0.700 | −0.396 | 0.258 | 0.080 | −0.178 | −0.248 |
| ADORA2B-Tango | −0.194 | −0.877 | −0.210 | −0.134 | −0.087 | −0.462 | −0.285 |
| ADORA3-Tango | −0.036 | −0.379 | −0.060 | 0.066 | 0.016 | −0.245 | −0.373 |
| ADRA1A-Tango | 0.093 | −0.389 | −0.118 | 0.664 | −0.023 | −0.282 | −0.268 |
| ADRA1B-Tango | −0.692 | −1.055 | −0.200 | 0.295 | −0.213 | −0.351 | −0.494 |
| ADRA1D-Tango | −0.292 | −0.244 | −0.318 | 0.731 | −0.450 | −0.754 | −0.543 |
| ADRA2A-Tango | −0.410 | −0.129 | −0.490 | 3.260 | 0.375 | 0.226 | 0.236 |
| ADRA2B-Tango | −1.033 | −0.788 | −1.467 | 1.971 | 0.009 | −0.442 | −0.273 |
| ADRA2C-Tango | 0.477 | 0.631 | −0.125 | 3.097 | 0.733 | 0.367 | 0.304 |
| ADRB1-Tango | −0.383 | 0.559 | 0.165 | 2.055 | 0.961 | 0.982 | 0.877 |
| ADRB2-Tango | −0.528 | −0.047 | 0.054 | 0.463 | 0.663 | −0.028 | 0.709 |
| ADRB3-Tango | 0.204 | −0.812 | −0.022 | 0.452 | 0.262 | 0.280 | 0.207 |
| AGTR1-Tango | 0.157 | 0.298 | −0.125 | 0.334 | 0.370 | 0.499 | 0.313 |
| AGTR2-Tango | 0.051 | 0.689 | 0.185 | 0.932 | 0.735 | 0.628 | 0.625 |
| APJ-Tango | −0.441 | −0.297 | −0.275 | 1.096 | 0.655 | 0.644 | 0.175 |
| AVPR1A-Tango | −0.520 | −0.940 | −0.296 | 0.097 | 0.086 | −0.191 | −0.494 |
| AVPR1B-Tango | −0.219 | −0.132 | 0.135 | 0.611 | 0.293 | 0.116 | −0.351 |
| AVPR2-Tango | −0.661 | 0.113 | −0.611 | 1.026 | 0.267 | −0.108 | −0.098 |
| BB3-Tango | −0.560 | −0.229 | −0.235 | 1.159 | 0.489 | 0.284 | 0.064 |
| BDKBR1-Tango | −0.057 | −0.426 | 0.172 | 1.408 | 0.671 | 0.540 | 0.342 |
| BDKBR2-Tango | 0.046 | 0.264 | 0.272 | 0.309 | 0.739 | 0.481 | 0.195 |
| C3AR1-Tango | −0.187 | −0.259 | −0.354 | 0.469 | 0.097 | 0.088 | 0.012 |
| C5A-Tango | −0.720 | −0.478 | 0.245 | 0.270 | 0.604 | 0.311 | 0.124 |
| CALCRb-Tango | −0.443 | −0.838 | −0.042 | −0.031 | −0.071 | −0.329 | −0.212 |
| CALCRL-Tango | −0.047 | −0.779 | −0.180 | −0.072 | 0.021 | 0.037 | −0.008 |
| CASR-Tango | −1.354 | −1.444 | −0.055 | 0.276 | 0.267 | 0.184 | −0.010 |
| CCKAR-Tango | −0.383 | −0.317 | −0.074 | −0.051 | 0.115 | 0.028 | −0.324 |
| CCKBR-Tango | −1.082 | −0.885 | −0.362 | 1.348 | 0.015 | 0.118 | −0.096 |
| CCR10-Tango | −1.091 | −0.398 | −0.255 | 0.444 | 0.159 | 0.310 | 0.114 |
| CCR2-Tango | −1.444 | −1.194 | −0.065 | 1.065 | 0.314 | 0.262 | 0.171 |
| CCR3-Tango | −0.326 | 1.462 | −0.672 | 0.795 | 0.313 | 0.050 | −0.244 |
| CCR4-Tango | −0.594 | 0.238 | −0.100 | 0.670 | 0.584 | 0.190 | 0.177 |
| CCR5-Tango | −0.029 | 0.784 | −0.095 | −0.033 | 0.357 | −0.039 | −0.230 |
| CCR6-Tango | 0.209 | 1.039 | 0.011 | 0.994 | 0.352 | 0.079 | −0.122 |
| CCR7-Tango | −0.292 | 0.208 | −0.219 | 0.069 | 0.156 | −0.174 | −0.185 |
| CCR8-Tango | −0.042 | 0.181 | 0.073 | 1.044 | 0.175 | −0.144 | −0.230 |
| CCRL2-Tango | 0.044 | −0.318 | −0.267 | 0.317 | 0.158 | 0.033 | 0.592 |
| CD97-Tango | −0.660 | 0.091 | −0.160 | 2.573 | 0.795 | 0.748 | 0.160 |
| CHRM1-Tango | 0.136 | −0.040 | −0.472 | 0.801 | 0.379 | 0.477 | −0.026 |
| CHRM2-Tango | −0.859 | −0.556 | 0.082 | 1.375 | 0.718 | 0.534 | 0.457 |
| CHRM3-Tango | −0.280 | 0.109 | −0.062 | 1.679 | 0.552 | 0.469 | 0.314 |
| CHRM4-Tango | 0.348 | −0.106 | 0.122 | 0.345 | 0.507 | 0.436 | 0.403 |
| CHRM5-Tango | 0.000 | 0.436 | 0.238 | 0.174 | 0.881 | 0.643 | 0.423 |
| CMKLR1-Tango | −0.304 | −0.607 | −0.033 | 0.341 | 0.328 | 0.384 | 0.226 |
| CNR1-Tango | −0.086 | −0.189 | −0.146 | 0.144 | 0.113 | −0.234 | −0.251 |
| CNR2-Tango | 0.055 | −0.257 | −0.345 | −0.047 | −0.047 | −0.012 | −0.362 |
| CRHR1-Tango | −0.160 | −0.407 | 0.000 | 0.802 | 0.167 | 0.188 | 0.147 |
| CRHR2-Tango | 0.445 | −0.047 | 0.118 | 0.491 | 0.207 | 0.129 | 0.140 |
| CX3C1-Tango | −0.069 | −0.445 | −0.102 | 0.521 | 0.514 | 0.302 | 0.082 |
| CXCR1-Tango | 0.620 | 0.602 | 0.239 | 0.455 | 0.624 | 0.557 | 0.224 |
| CXCR2-Tango | 1.019 | 1.652 | 0.490 | 0.448 | 1.035 | 0.887 | 0.648 |
| CXCR3-Tango | 0.205 | 0.282 | −0.050 | 0.835 | 0.788 | 0.513 | 0.313 |
| CXCR4-Tango | 0.185 | 0.660 | 0.118 | 0.244 | 0.424 | 0.467 | 0.359 |
| CXCR5-Tango | −0.010 | −0.559 | −0.032 | 0.785 | 0.371 | 0.398 | 0.413 |
| CXCR6-Tango | 1.235 | 1.310 | −0.010 | 0.946 | 0.859 | 0.588 | 0.693 |
| CXCR7-Tango | −0.206 | 0.896 | −0.411 | 1.194 | 0.945 | 0.980 | 0.653 |
| CYSLTR1-Tango | 0.731 | 1.218 | −0.284 | 0.621 | 0.492 | 0.487 | 0.148 |
| CYSLTR2-Tango | 0.570 | 0.150 | 0.311 | 0.890 | 0.775 | 0.738 | 0.473 |
| DRD1-Tango | 0.750 | 0.360 | −0.099 | 0.986 | 0.384 | 0.350 | 0.051 |
| DRD2-Tango | 0.629 | 0.506 | −0.073 | 4.161 | 1.146 | 0.789 | 3.080 |
| DRD3-Tango | 1.534 | 0.624 | 0.604 | 4.937 | 1.926 | 1.339 | 1.193 |
| DRD4-Tango | 0.655 | 1.040 | 0.877 | 5.482 | 2.078 | 0.887 | 1.028 |
| DRD5-Tango | 0.255 | 0.111 | −0.367 | 0.143 | −0.298 | −0.373 | −0.272 |
| EDNRA-Tango | −0.085 | 0.409 | 0.097 | 0.159 | 0.318 | 0.210 | −0.050 |
| EDNRB-Tango | −0.051 | −0.517 | −0.063 | 0.181 | 0.496 | 0.173 | 0.249 |
| ELTD1-Tango | −0.788 | −0.521 | −0.334 | 0.803 | 0.586 | 0.361 | 0.141 |
| F2R-Tango | −0.042 | −0.243 | −0.200 | 0.156 | −0.027 | 0.058 | −0.350 |
| F2RL1-Tango | −0.039 | −0.230 | −0.090 | 0.003 | 0.177 | 0.031 | −0.183 |
| F2RL2-Tango | −0.285 | −0.009 | −0.089 | 0.014 | −0.035 | −0.050 | −0.257 |
| F2RL3-Tango | −0.135 | 0.000 | −0.141 | 0.232 | 0.049 | −0.116 | −0.141 |
| FFA1-Tango | −0.423 | 0.051 | 0.152 | 0.642 | 0.718 | 0.542 | 0.513 |
| FFA2-Tango | −0.400 | 0.176 | −0.242 | 0.837 | 0.231 | 0.021 | −0.013 |
| FFA3-Tango | −0.023 | 0.384 | 0.059 | 0.678 | 0.254 | 0.555 | 0.471 |
| FPR1-Tango | 0.540 | 1.109 | 0.644 | 1.231 | 1.381 | 1.147 | 1.013 |
| FPR2-Tango | 0.111 | 0.313 | 0.483 | 2.992 | 0.836 | 0.508 | 0.301 |
| FPR3-Tango | −0.334 | 0.402 | −0.270 | 0.574 | 0.266 | 0.145 | 0.144 |
| FSHR-Tango | −0.539 | −0.262 | −0.202 | 0.194 | 0.221 | 0.118 | −0.234 |
| GABBR1-Tango | 0.038 | −0.284 | 0.024 | 2.995 | 0.513 | 0.241 | 0.176 |
| GAL1-Tango | −0.646 | −1.222 | −0.293 | 1.984 | 0.700 | 0.225 | 0.412 |
| GAL2-Tango | 0.885 | 0.254 | −0.072 | 0.670 | 0.102 | −0.208 | −0.392 |
| GAL3-Tango | −0.707 | −0.491 | −0.147 | 1.518 | −0.280 | 0.170 | −0.120 |
| GCGR-Tango | −0.521 | −0.471 | −0.402 | 0.553 | 0.077 | −0.402 | −0.402 |
| GHRHR-Tango | −0.336 | −0.575 | 0.198 | 0.679 | 0.234 | 0.118 | 0.234 |
| GHSR-Tango | −0.247 | −0.217 | −0.816 | 0.470 | 0.020 | −0.234 | −0.294 |
| GIPR-Tango | −1.122 | −0.840 | −0.455 | 0.091 | −0.455 | −0.467 | −0.591 |
| GLP1R-Tango | −0.592 | −0.958 | −0.705 | −0.065 | −0.504 | −0.914 | −1.447 |
| GLP2R-Tango | −0.029 | −0.116 | −0.325 | 0.142 | −0.136 | −0.164 | −0.378 |
| GNRHR-Tango | 0.148 | 0.094 | 0.221 | 0.141 | 0.051 | 0.327 | 0.337 |
| GPBA-Tango | −0.532 | 0.855 | 0.000 | 1.069 | 0.328 | 0.118 | 0.298 |
| GPER-Tango | −0.191 | 0.146 | 0.125 | 1.039 | 0.412 | 0.252 | 0.176 |
| GPR1-Tango | −0.547 | 0.128 | −0.191 | 0.650 | 0.424 | 0.424 | 0.281 |
| GPR101-Tango | −0.163 | 0.271 | 0.137 | 0.530 | 0.520 | 0.369 | 0.226 |
| GPR110-Tango | −0.201 | −0.354 | −0.434 | 0.814 | −0.172 | −0.418 | −0.563 |
| GPR111-Tango | −0.108 | −0.417 | 0.102 | 0.776 | 0.429 | 0.076 | −0.070 |
| GPR113-Tango | −0.185 | −0.166 | −0.152 | 0.138 | −0.206 | −0.226 | −0.491 |
| GPR114-Tango | −0.436 | −0.634 | −0.108 | 0.248 | 0.179 | 0.079 | −0.036 |
| GPR115-Tango | −0.455 | −0.173 | −0.346 | 0.134 | 0.026 | −0.023 | −0.371 |
| GPR116-Tango | −0.671 | −0.496 | −0.405 | 1.028 | 0.042 | −0.100 | −0.377 |
| GPR119-Tango | 0.114 | −0.123 | 0.013 | 0.462 | 0.212 | 0.055 | −0.038 |
| GPR12-Tango | −0.380 | 0.310 | 0.096 | 1.154 | 0.357 | 0.133 | 0.050 |
| GPR120-Tango | −0.355 | −0.571 | 0.008 | 1.719 | 0.466 | 0.448 | 0.514 |
| GPR123-Tango | 0.196 | 0.047 | −0.067 | 0.825 | 0.123 | −0.193 | −0.196 |
| GPR124-Tango | −0.272 | −0.025 | −0.185 | 0.104 | 0.214 | 0.052 | −0.024 |
| GPR125-Tango | −0.010 | 0.157 | −0.295 | −0.253 | 0.218 | 0.073 | −0.085 |
| GPR126-Tango | −0.502 | −0.565 | −0.229 | 0.879 | 0.077 | −0.159 | −0.127 |
| GPR132-Tango | −0.762 | −0.667 | −0.551 | 0.482 | −0.507 | −0.371 | −0.647 |
| GPR133-Tango | −0.458 | −0.331 | −0.499 | −0.077 | 0.032 | −0.111 | −0.391 |
| GPR135-Tango | −0.289 | −0.343 | 0.043 | 0.255 | 0.195 | 0.037 | −0.116 |
| GPR141-Tango | −0.570 | 0.162 | 0.484 | 1.873 | 0.858 | 0.541 | 0.555 |
| GPR142-Tango | −0.225 | −0.065 | −0.119 | 0.301 | 0.179 | 0.019 | −0.180 |
| GPR143-Tango | −0.560 | 0.044 | −0.145 | 0.000 | 0.099 | −0.090 | −0.496 |
| GPR144-Tango | −1.718 | −0.315 | 0.040 | 0.155 | 0.453 | 0.024 | 0.064 |
| GPR146-Tango | −0.217 | 0.246 | −0.477 | 0.405 | −0.062 | 0.000 | −0.395 |
| GPR148-Tango | 0.280 | 0.684 | 0.160 | 0.631 | 0.282 | 0.082 | 0.234 |
| GPR149-Tango | 0.000 | 0.670 | −0.155 | 0.932 | 0.262 | 0.489 | 0.193 |
| GPR15-Tango | −0.121 | 0.350 | −0.311 | 0.938 | −0.127 | −0.564 | −0.192 |
| GPR150-Tango | 0.112 | 0.000 | −0.037 | 0.954 | −0.216 | −0.037 | 0.204 |
| GPR151-Tango | −0.592 | 0.212 | 0.524 | 0.459 | 0.501 | 0.392 | 0.347 |
| GPR152-Tango | −0.419 | −0.739 | −0.096 | 0.499 | 0.253 | 0.357 | 0.105 |
| GPR153-Tango | −0.136 | 0.239 | −0.191 | 0.134 | 0.447 | 0.332 | 0.166 |
| GPR156-Tango | −0.343 | −0.148 | −0.132 | 1.433 | 0.274 | 0.328 | 0.094 |
| GPR157-Tango | −0.061 | −0.050 | −0.025 | 0.791 | 0.442 | 0.255 | 0.377 |
| GPR158-Tango | −0.400 | −0.526 | −0.126 | 0.526 | −0.102 | 0.130 | −0.140 |
| GPR160-Tango | 0.000 | 0.205 | 0.210 | 0.278 | 0.353 | 0.043 | −0.066 |
| GPR161-Tango | −0.061 | 0.538 | 0.224 | 0.435 | 0.246 | 0.237 | 0.014 |
| GPR162-Tango | −0.424 | −0.471 | −0.142 | 0.255 | 0.004 | −0.109 | −0.145 |
| GPR17-Tango | 0.289 | 0.515 | 0.351 | 0.747 | 0.664 | 0.670 | 0.632 |
| GPR171-Tango | −0.165 | 0.058 | 0.096 | 0.754 | 0.277 | 0.560 | 0.455 |
| GPR173-Tango | −0.489 | −0.184 | 0.209 | 0.561 | 0.535 | 0.614 | 0.407 |
| GPR174-Tango | 0.061 | 0.428 | −0.095 | 0.674 | 0.788 | 0.625 | 0.564 |
| GPR18-Tango | −0.009 | −0.249 | −0.020 | 0.493 | 0.544 | 0.410 | 0.372 |
| GPR182-Tango | −0.263 | 0.211 | −0.172 | 0.293 | 0.630 | 0.620 | 0.407 |
| GPR183-Tango | −0.369 | −0.592 | 0.059 | 0.487 | 0.609 | 0.556 | 0.441 |
| GPR19-Tango | −0.330 | −0.372 | −0.001 | 0.333 | 0.560 | 0.219 | −0.068 |
| GPR20-Tango | 0.170 | −0.368 | −0.263 | 0.737 | 0.350 | 0.328 | 0.328 |
| GPR21-Tango | −0.405 | −0.648 | 0.372 | 0.469 | 0.017 | −0.318 | 0.232 |
| GPR22-Tango | 0.358 | 0.295 | 0.204 | 0.148 | 0.543 | 0.168 | −0.054 |
| GPR25-Tango | 0.191 | −0.542 | 0.385 | 0.863 | 0.577 | 0.910 | 0.526 |
| GPR26-Tango | −1.410 | −0.564 | −0.440 | 0.947 | 0.142 | 0.472 | 0.198 |
| GPR27-Tango | 0.266 | 0.501 | 0.326 | 0.547 | 0.651 | 0.748 | 0.781 |
| GPR3-Tango | −0.033 | 0.722 | 0.112 | 0.655 | 0.536 | 0.646 | 0.716 |
| GPR31-Tango | −0.289 | 0.431 | −0.326 | 0.791 | 0.115 | 0.186 | 0.247 |
| GPR32-Tango | −0.437 | −0.080 | −0.889 | 0.482 | 0.146 | −0.201 | −0.461 |
| GPR34-Tango | −0.766 | 0.300 | −0.308 | −0.203 | −0.125 | −0.299 | −0.136 |
| GPR35-Tango | −0.688 | 0.480 | 0.301 | 1.155 | 0.704 | 0.485 | 0.490 |
| GPR37-Tango | −0.280 | 0.622 | −0.221 | 1.371 | 0.288 | 0.499 | 0.358 |
| GPR37L1-Tango | −0.285 | −0.360 | −0.234 | 1.262 | 0.015 | −0.145 | −0.465 |
| GPR39-Tango | −0.743 | −0.765 | 0.149 | 0.408 | 0.521 | 0.547 | 0.817 |
| GPR4-Tango | −0.176 | 0.775 | 0.060 | 0.328 | 0.516 | 0.697 | 0.388 |
| GPR44-Tango | −0.166 | −0.096 | −0.157 | 0.070 | 0.120 | 0.218 | 0.193 |
| GPR45-Tango | −0.106 | −0.918 | −0.436 | 0.987 | −0.019 | 0.031 | −0.131 |
| GPR50-Tango | 0.720 | −0.258 | −0.105 | 0.171 | 0.067 | 0.044 | 0.000 |
| GPR52-Tango | 0.379 | −0.370 | 0.388 | 1.352 | 0.816 | 0.532 | 0.803 |
| GPR55-Tango | −0.516 | −0.566 | −0.159 | 1.362 | 0.135 | 0.385 | 0.177 |
| GPR56-Tango | −0.176 | −0.759 | −0.180 | 1.193 | −0.363 | −0.576 | −0.941 |
| GPR6-Tango | −0.248 | −0.696 | 0.283 | 0.376 | 0.392 | 0.319 | 0.318 |
| GPR61-Tango | 0.183 | −0.073 | 0.284 | 0.531 | 0.536 | 0.528 | 0.503 |
| GPR62-Tango | −0.896 | −0.896 | −0.120 | 0.086 | −0.400 | −0.091 | −0.174 |
| GPR63-Tango | 0.027 | −0.594 | −0.176 | 0.607 | 0.738 | 0.142 | 0.136 |
| GPR64-Tango | −0.174 | −0.269 | −0.413 | 0.315 | 0.335 | 0.004 | 0.016 |
| GPR65-Tango | 0.122 | −0.191 | −0.306 | −0.030 | −0.074 | −0.103 | −0.134 |
| GPR68-Tango | 0.035 | −0.580 | −0.711 | −0.208 | −0.493 | −0.536 | −0.717 |
| GPR75-Tango | 0.392 | −0.490 | −0.477 | 0.498 | 0.134 | −0.412 | −0.389 |
| GPR77-Tango | −1.013 | −0.286 | −0.656 | 0.684 | −0.031 | −0.260 | −0.252 |
| GPR78-Tango | −1.025 | −0.344 | 0.024 | 0.591 | 0.414 | 0.240 | 0.299 |
| GPR82-Tango | −0.566 | 0.671 | −0.766 | 0.389 | 0.212 | −0.313 | −0.340 |
| GPR83-Tango | −0.424 | −0.566 | 0.016 | 1.313 | 0.544 | 0.101 | 0.124 |
| GPR84-Tango | −0.616 | 0.269 | −0.466 | 1.016 | 0.609 | 0.162 | 0.034 |
| GPR85-Tango | −0.226 | −0.870 | −0.074 | 0.681 | 0.253 | 0.264 | 0.226 |
| GPR87-Tango | −0.273 | 0.315 | −0.129 | 0.427 | 0.572 | 0.340 | 0.281 |
| GPR88-Tango | −0.076 | −0.754 | 0.015 | 0.540 | 0.432 | 0.264 | 0.184 |
| GPR97-Tango | −0.617 | −0.058 | −0.288 | 0.945 | 0.264 | −0.010 | −0.141 |
| GPRC5A-Tango | −0.886 | −0.567 | −0.419 | 0.942 | 0.074 | 0.026 | 0.013 |
| GPRC5B-Tango | −0.388 | −0.450 | −0.129 | 0.461 | 0.360 | 0.122 | 0.097 |
| GPRC5C-Tango | 0.218 | 0.368 | −0.014 | 0.411 | 0.123 | −0.041 | 0.131 |
| GPRC5D-Tango | −0.262 | −0.869 | −0.321 | 0.983 | 0.030 | −0.064 | −0.042 |
| GPRC6A-Tango | 0.020 | −0.476 | −0.131 | 0.072 | 0.038 | −0.089 | −0.243 |
| GRM1-Tango | 0.099 | −0.423 | 0.077 | 0.358 | 0.024 | −0.147 | −0.332 |
| GRM2-Tango | −0.510 | −0.720 | −0.027 | 1.328 | −0.225 | −0.348 | −0.170 |
| GRM4-Tango | −0.148 | −0.468 | −0.113 | 0.597 | −0.111 | −0.353 | −0.507 |
| GRM5-Tango | −0.132 | −0.451 | −0.245 | 0.299 | −0.205 | −0.297 | −0.448 |
| GRM6-Tango | 0.000 | 0.000 | −0.204 | 0.166 | 0.235 | 0.294 | 0.166 |
| GRM7-Tango | −0.144 | 0.232 | −0.517 | −0.169 | −0.169 | −0.169 | −0.336 |
| GRM8-Tango | −0.250 | −0.862 | −0.305 | 0.953 | −0.188 | −0.068 | −0.630 |
| GRPR-Tango | −0.127 | −0.219 | −0.359 | 0.153 | 0.130 | −0.257 | −0.288 |
| HCA1-Tango | −0.327 | −0.628 | −0.246 | 0.582 | 0.382 | 0.465 | 0.153 |
| HCA2-Tango | −0.012 | 0.792 | −0.019 | 1.527 | 0.595 | 0.658 | 0.518 |
| HCA3-Tango | 0.127 | 0.065 | −0.376 | 0.716 | 0.470 | 0.280 | 0.462 |
| HCTR1-Tango | −0.778 | −0.159 | 0.261 | 0.482 | 0.712 | 0.322 | 0.292 |
| HCTR2-Tango | −0.766 | 0.339 | 0.087 | 0.273 | −0.281 | −0.497 | −0.126 |
| HRH1-Tango | 0.245 | 0.508 | 0.276 | 0.426 | 1.106 | 0.723 | 0.823 |
| HRH2-Tango | 0.382 | 0.045 | 0.281 | 0.544 | 0.665 | 0.751 | 1.018 |
| HRH3-Tango | 0.960 | 1.303 | 0.961 | 0.715 | 1.444 | 1.148 | 2.341 |
| HRH4-Tango | 0.782 | 1.451 | 0.726 | 0.671 | 1.567 | 1.281 | 2.012 |
| HTR1A-Tango | 0.345 | 0.298 | 0.058 | 1.479 | 0.498 | 0.184 | 0.352 |
| HTR1B-Tango | 0.708 | 0.907 | 0.110 | 0.694 | 0.669 | 0.545 | 0.400 |
| HTR1D-Tango | 0.142 | 0.434 | 0.122 | 0.620 | 0.453 | 0.263 | 0.359 |
| HTR1E-Tango | 0.061 | 0.661 | 0.052 | 0.459 | 0.484 | 0.379 | 0.238 |
| HTR1F-Tango | 0.172 | 0.343 | −0.256 | 0.353 | 0.037 | 0.020 | 0.177 |
| HTR2A-Tango | −0.136 | 0.025 | −0.354 | 1.172 | 0.271 | 0.155 | 0.049 |
| HTR2B-Tango | 0.143 | 0.235 | −0.109 | 0.516 | 0.234 | 0.455 | 0.138 |
| HTR2C-Tango | −0.512 | −0.939 | −0.164 | 1.055 | 0.161 | 0.035 | 0.029 |
| HTR4-Tango | 0.057 | −0.141 | 0.312 | 1.445 | 0.577 | 0.507 | 0.467 |
| HTR5-Tango | 0.320 | 0.817 | −0.277 | 0.595 | 0.319 | 0.254 | 0.213 |
| HTR6-Tango | −0.133 | −0.460 | 0.019 | 1.115 | 0.520 | 0.471 | 0.573 |
| HTR7-Tango | −0.033 | 0.130 | −0.170 | 1.024 | 0.410 | 0.328 | 0.216 |
| KISSPEPTIN-R- | −0.596 | −0.596 | 0.496 | 0.378 | 0.219 | 0.451 | −0.194 |
| Tango | | | | | | | |
| LHCGR-Tango | −0.647 | −1.177 | −0.086 | 0.394 | 0.153 | 0.120 | −0.075 |
| LPAR1-Tango | −0.458 | 0.189 | 0.182 | 1.022 | 0.736 | 0.903 | 0.708 |
| LPAR2-Tango | −0.766 | −1.120 | 0.081 | 0.469 | 0.469 | 0.333 | 0.326 |
| LPAR4-Tango | −0.759 | −0.849 | −0.196 | 0.789 | 0.304 | 0.342 | 0.066 |
| LPAR5-Tango | −0.416 | −0.677 | 0.276 | 1.460 | 0.636 | 0.523 | 0.382 |
| LPAR6-Tango | 0.543 | 0.338 | 0.130 | 0.321 | 0.234 | 0.197 | 0.290 |
| LTB4R-Tango | 0.234 | −0.194 | −0.103 | 0.528 | 0.242 | 0.284 | 0.000 |
| LTB4R2B-Tango | −0.115 | 0.197 | 0.029 | 0.079 | 0.342 | −0.031 | −0.129 |
| MAS1-Tango | 0.016 | 0.054 | −0.337 | 0.566 | 0.412 | 0.170 | 0.155 |
| MAS1L-Tango | −0.613 | −0.329 | −0.053 | −0.021 | 0.347 | 0.247 | −0.036 |
| MC1R-Tango | −0.549 | −0.713 | 0.153 | 0.455 | 0.257 | 0.127 | 0.167 |
| MC2R-Tango | 0.265 | 0.165 | 0.328 | 0.676 | 0.392 | 0.000 | 0.248 |
| MC3R-Tango | −0.025 | 0.112 | 0.245 | 0.916 | 0.541 | −0.245 | 0.280 |
| MC4R-Tango | −0.295 | 0.140 | 0.141 | 1.538 | 0.679 | 0.566 | 0.653 |
| MC5R-Tango | −0.672 | 0.255 | −0.057 | 0.633 | 0.788 | 0.336 | 0.170 |
| MCHR1-Tango | −0.383 | 0.093 | 0.047 | 0.573 | 0.556 | 0.226 | 0.182 |
| MCHR2-Tango | −0.328 | −0.184 | −0.191 | −0.025 | 0.112 | −0.088 | −0.121 |
| MLNR-Tango | 0.075 | −0.413 | −0.128 | 0.273 | 0.243 | 0.230 | 0.168 |
| MRGPRD-Tango | 0.031 | 0.366 | −0.223 | 0.226 | 0.502 | 0.133 | 0.171 |
| MRGPRE-Tango | 0.024 | −0.147 | 0.563 | 0.636 | 0.557 | 0.571 | 0.356 |
| MRGPRF-Tango | −0.105 | 0.056 | 0.120 | 1.195 | 1.074 | 0.909 | 0.942 |
| MRGPRG-Tango | −0.133 | −0.039 | 0.072 | 0.148 | 0.616 | 0.779 | 0.599 |
| MRGPRX1-Tango | −0.137 | 0.094 | −0.053 | 0.779 | 0.470 | 0.224 | 0.136 |
| MRGPRX2-Tango | −0.296 | 0.536 | 0.098 | 1.086 | 0.638 | 0.401 | 0.349 |
| MRGPRX3-Tango | 0.295 | −0.298 | 0.185 | 0.621 | 0.344 | 0.306 | 0.260 |
| MRGPRX4-Tango | −0.799 | 0.057 | −1.082 | 1.117 | 0.149 | −0.713 | −0.968 |
| MTNR1A-Tango | −0.660 | 0.444 | −0.238 | 1.103 | 0.806 | 0.364 | 0.239 |
| MTNR1B-Tango | −0.405 | 0.303 | −0.511 | 2.202 | 0.575 | 0.407 | 0.272 |
| NMBR-Tango | 0.085 | 0.064 | −0.150 | 1.157 | 0.851 | 0.288 | 0.355 |
| NMUR1-Tango | −0.263 | −0.840 | 0.164 | 0.379 | 0.307 | 0.089 | −0.321 |
| NMUR2-Tango | −0.874 | 0.710 | −0.347 | 0.346 | 0.034 | −0.028 | −0.548 |
| NPBW1-Tango | 0.068 | −0.199 | 0.081 | 0.964 | 0.855 | 0.018 | −0.145 |
| NPBW2-Tango | −0.090 | −0.082 | −1.050 | 1.433 | 0.553 | −0.086 | −0.239 |
| NPFF1-Tango | 0.057 | −1.012 | 0.293 | 1.033 | 0.854 | 0.410 | 0.425 |
| NPFF2-Tango | −0.372 | −0.115 | −0.135 | 0.390 | 0.447 | −0.102 | 0.139 |
| NPS-Tango | −0.481 | −1.175 | −0.326 | −0.042 | −0.035 | −0.461 | −0.152 |
| NPY1R-Tango | −0.742 | −0.966 | −0.051 | 0.436 | 0.699 | 0.139 | 0.011 |
| NPY2R-Tango | −0.384 | −0.645 | −0.530 | 0.161 | 0.116 | −0.263 | −0.227 |
| NPY4R-Tango | −0.235 | −0.798 | 0.012 | −0.126 | −0.163 | −0.177 | −0.260 |
| NPY5R-Tango | −0.356 | −0.098 | −0.135 | 1.087 | 0.454 | 0.316 | 0.254 |
| NTSR1-Tango | −0.143 | 0.468 | −0.339 | 0.552 | 0.622 | 0.288 | 0.341 |
| NTSR2-Tango | −0.386 | −0.520 | −0.348 | 0.338 | 0.194 | 0.009 | −0.097 |
| OPN3-Tango | −0.258 | −0.455 | 0.044 | 0.555 | 0.286 | 0.131 | 0.051 |
| OPN5-Tango | −0.720 | 0.064 | −0.475 | 1.664 | −0.158 | −0.496 | −0.709 |
| OPRD1-Tango | −0.656 | −0.453 | −0.785 | 0.906 | −0.215 | −0.428 | −0.489 |
| OPRK1-Tango | −0.058 | −0.024 | 0.059 | 0.693 | 0.294 | 0.443 | 0.269 |
| 0PRL1-Tango | 0.846 | 0.505 | −0.035 | 0.486 | 0.196 | −0.122 | 0.063 |
| OPRM1-Tango | −0.558 | −0.518 | −0.285 | 1.466 | 0.190 | 0.339 | −0.195 |
| OXER1-Tango | −0.598 | 0.133 | 0.121 | 0.549 | 0.453 | 0.226 | 0.171 |
| OXGR1-Tango | −0.913 | 0.234 | −0.609 | 1.142 | 0.231 | −0.020 | −0.297 |
| OXTR-Tango | −0.042 | −0.692 | 0.125 | 0.650 | 0.563 | 0.393 | 0.295 |
| P2RY1-Tango | −0.295 | 0.267 | −0.424 | 0.944 | 0.393 | 0.272 | 0.381 |
| P2RY10-Tango | −0.314 | −0.392 | −0.080 | 1.065 | 0.370 | 0.214 | 0.197 |
| P2RY11-Tango | −0.193 | −0.658 | 0.124 | 0.739 | 0.433 | 0.124 | 0.085 |
| P2RY12-Tango | −0.068 | 0.860 | −0.231 | 1.820 | 0.780 | 0.575 | 0.488 |
| P2RY13-Tango | −0.127 | 0.670 | 0.056 | 0.599 | 0.358 | 0.318 | 0.280 |
| P2RY14-Tango | −0.111 | 0.749 | −0.067 | 0.569 | 0.391 | 0.276 | 0.297 |
| P2RY2-Tango | −0.417 | −0.228 | −0.065 | 0.735 | 0.317 | 0.239 | 0.091 |
| P2RY4-Tango | −0.341 | 0.225 | −0.313 | 0.901 | 0.229 | 0.111 | 0.001 |
| P2RY6-Tango | −0.096 | 0.489 | −0.045 | 0.905 | 0.211 | 0.144 | 0.162 |
| P2RY8-Tango | −0.093 | −0.023 | −0.191 | 0.665 | 0.168 | −0.073 | 0.003 |
| PK1-Tango | −0.183 | −0.619 | 0.086 | 0.384 | 0.258 | 0.265 | 0.265 |
| PK2-Tango | −0.216 | −0.036 | 0.440 | 0.314 | 0.341 | 0.050 | 0.263 |
| PRRP-Tango | 0.138 | −0.026 | −0.371 | 0.778 | 0.287 | 0.140 | −0.099 |
| PTAFR-Tango | 0.231 | 0.617 | −0.218 | 0.615 | 0.585 | 0.549 | 0.355 |
| PTGDR-Tango | −0.098 | −0.956 | 0.063 | 0.846 | 0.232 | 0.128 | 0.000 |
| PTGER1-Tango | −0.044 | −0.722 | −0.485 | 1.238 | 0.037 | 0.045 | −0.063 |
| PTGER2-Tango | 0.272 | 0.150 | 0.362 | 1.314 | 0.938 | 0.676 | 0.284 |
| PTGER3-Tango | 0.019 | 0.019 | 0.179 | 1.363 | 0.530 | 0.634 | 0.363 |
| PTGER4-Tango | −0.156 | 0.123 | 0.348 | 0.902 | 0.780 | 0.906 | 0.860 |
| PTGFR-Tango | 0.310 | 0.117 | −0.366 | 0.904 | 0.622 | 0.808 | 0.395 |
| PTGIR-Tango | −0.122 | 0.623 | −0.122 | −0.170 | −0.957 | −0.675 | −1.138 |
| PTH1R-Tango | 0.335 | −0.164 | −0.240 | 0.465 | 0.262 | 0.174 | 0.109 |
| PTH2R-Tango | 0.313 | 0.617 | −0.219 | 0.967 | 0.115 | −0.119 | −0.391 |
| QRFP-Tango | 0.111 | 0.214 | 0.098 | 0.026 | 0.143 | 0.187 | −0.245 |
| RXFP1-Tango | −0.454 | −0.758 | 0.098 | 0.287 | −0.234 | −0.369 | −0.460 |
| RXFP2-Tango | −0.281 | −0.101 | −0.130 | 0.312 | −0.025 | 0.136 | −0.246 |
| RXFP3-Tango | −0.681 | −0.974 | −0.319 | 0.156 | 0.095 | −0.426 | −0.280 |
| RXFP4-Tango | 0.000 | 0.405 | 0.162 | −0.214 | 0.020 | −0.144 | −0.280 |
| S1PR1-Tango | −0.309 | 0.179 | 0.792 | 1.324 | 0.686 | 0.575 | 0.717 |
| S1PR2-Tango | −0.877 | 0.211 | 0.296 | 0.919 | 0.396 | 0.267 | 0.388 |
| S1PR3-Tango | −0.160 | 0.144 | 0.130 | 0.522 | 0.261 | −0.034 | −0.011 |
| S1PR4-Tango | −0.873 | 0.904 | −0.224 | 1.311 | 0.373 | 0.004 | 0.040 |
| S1PR5-Tango | 0.345 | 0.611 | 0.447 | 0.186 | 0.517 | −0.103 | −0.126 |
| SCTR-Tango | 0.722 | 1.861 | 0.273 | 1.414 | 1.078 | 0.678 | 0.874 |
| SSTR1-Tango | −0.282 | 0.415 | 0.255 | 0.873 | 1.224 | 0.363 | 0.675 |
| SSTR2-Tango | −0.133 | −0.503 | −0.298 | −0.079 | 0.000 | 0.000 | −0.474 |
| SSTR3-Tango | 0.697 | 1.057 | 0.500 | 0.484 | 0.484 | −0.206 | 0.087 |
| SSTR4-Tango | 0.303 | 0.397 | −0.276 | 0.837 | 0.025 | −0.174 | −0.047 |
| SSTR5-Tango | 0.742 | 0.529 | 0.042 | 0.714 | 0.737 | 0.246 | 0.354 |
| SUCNR1-Tango | 2.847 | 3.291 | 2.872 | 1.134 | 3.876 | 0.185 | −0.125 |
| TA1-Tango | 0.642 | 0.534 | −0.090 | 0.589 | 0.043 | −0.300 | −0.077 |
| TAAR2-Tango | 0.284 | −0.404 | 0.049 | 0.042 | 0.008 | 0.104 | −0.156 |
| TAAR5-Tango | −0.373 | −0.661 | −0.354 | −0.431 | −0.273 | −0.237 | −0.258 |
| TAAR6-Tango | −0.568 | 0.025 | −0.541 | −0.541 | −0.088 | −0.479 | −0.071 |
| TAAR8-Tango | −0.379 | −0.578 | −0.039 | 0.087 | 0.373 | 0.528 | 0.129 |
| TAAR9-Tango | −0.581 | −0.809 | −0.030 | 0.353 | 0.495 | 0.450 | 0.179 |
| TACR1-Tango | −0.094 | 0.474 | 0.318 | 1.168 | 0.689 | 0.697 | 0.557 |
| TACR2-Tango | −0.125 | −0.280 | 0.317 | 1.122 | 0.731 | 0.731 | 0.387 |
| TACR3-Tango | −0.618 | 0.278 | −0.236 | 1.012 | 0.364 | 0.156 | 0.032 |
| TBXA2R-Tango | −0.302 | −0.358 | 0.154 | 0.317 | 0.442 | 0.081 | 0.081 |
| TSHR-Tango | −0.752 | −0.803 | −0.562 | −0.187 | −0.534 | −0.609 | −0.886 |
| UTS2R-Tango | −0.969 | −1.447 | −0.246 | 0.051 | −0.115 | −0.525 | −0.413 |
| VIPR1-Tango | −1.033 | −0.822 | 0.091 | 1.576 | 0.232 | 0.633 | 0.341 |
| VIPR2-Tango | 1.600 | 2.101 | 0.177 | 1.014 | 1.105 | 1.118 | 1.447 |

TABLE 3
PRESTO-Tango with Gifu Medium

| | | Erysipelo- | Erysipelo- | Erysipelo- | | | | |
|---|---|---|---|---|---|---|---|---|
| PRESTO-Tango | Gifu | trichaceae | trichaceae | trichaceae | Ruminococcus | Ruminococcus | Ruminococcus | Blautia |
| with Gifu Medium | Medium | spp-1# | spp-2# | spp-3# | gnavus-1# | gnavus-2# | torques | producta |
| GPR1-Tango | 0 | 0.411 | 0.307 | 0.319 | 0.978 | 0.562 | −0.184 | 0.156 |
| GPR101-Tango | 0 | 0.544 | 0.454 | 0.671 | 0.603 | 0.682 | 0.225 | 0.033 |
| GPR110-Tango | 0 | 0.292 | 0.262 | 0.203 | −0.025 | 0.065 | 0.220 | 0.125 |
| GPR111-Tango | 0 | 0.745 | 0.373 | 0.252 | 0.786 | 0.252 | 0.286 | 0.341 |
| GPR113-Tango | 0 | −0.127 | 0.171 | −0.947 | 0.448 | 0.046 | −0.149 | −0.384 |
| GPR114-Tango | 0 | −0.420 | 0.515 | 0.325 | 0.759 | −0.186 | 0.165 | −0.554 |
| GPR115-Tango | 0 | −0.014 | −0.100 | −0.570 | 0.028 | 0.070 | −0.108 | −0.057 |
| GPR116-Tango | 0 | 0.250 | 0.263 | −0.039 | 0.301 | 0.291 | 0.165 | 0.151 |
| GPR119-Tango | 0 | 0.911 | 0.189 | 1.029 | −0.152 | 0.566 | −0.059 | 0.310 |
| GPR12-Tango | 0 | −0.232 | 0.106 | 0.141 | 1.208 | 0.828 | −0.508 | −0.124 |
| GPR120-Tango | 0 | 0.878 | −0.122 | 1.112 | 1.463 | 1.000 | −0.122 | 1.463 |
| GPR123-Tango | 0 | 0.590 | 0.590 | 0.126 | 0.403 | 0.340 | −0.376 | 0.298 |
| GPR124-Tango | 0 | −0.048 | 0.492 | 1.405 | 1.165 | 0.927 | 0.047 | 0.122 |
| GPR125-Tango | 0 | −0.103 | −0.154 | 0.211 | −0.067 | −0.097 | −0.145 | −0.260 |
| GPR126-Tango | 0 | 0.333 | 0.588 | 1.096 | 0.688 | 0.620 | −0.412 | 0.008 |
| GPR132-Tango | 0 | −0.168 | −0.139 | −0.788 | −0.666 | −0.557 | 0.444 | −0.258 |
| GPR133-Tango | 0 | 0.064 | 0.129 | −0.412 | −0.018 | 0.123 | −0.047 | −0.067 |
| GPR135-Tango | 0 | 0.053 | 0.218 | −0.383 | −0.231 | −0.156 | −0.674 | −0.846 |
| GPR141-Tango | 0 | 0.200 | −0.104 | −0.449 | 1.532 | 0.971 | −0.449 | −0.449 |
| GPR142-Tango | 0 | 0.293 | 0.509 | −0.209 | 0.960 | 0.415 | 0.920 | 0.472 |
| GPR143-Tango | 0 | 0.235 | 0.312 | −1.274 | 0.037 | −0.109 | 0.297 | 0.129 |
| GPR144-Tango | 0 | −0.032 | 0.323 | 2.738 | −0.560 | −0.272 | 0.196 | −0.112 |
| GPR146-Tango | 0 | −0.234 | 0.000 | 0.000 | 1.350 | 0.485 | −0.234 | 0.000 |
| GPR148-Tango | 0 | 0.783 | 0.274 | −0.104 | 0.381 | 0.381 | 0.528 | −0.104 |
| GPR149-Tango | 0 | 1.034 | 1.126 | 2.198 | 1.034 | 0.899 | 0.336 | 0.695 |
| GPR15-Tango | 0 | −0.252 | 0.392 | −0.381 | −0.556 | −0.506 | −1.012 | −1.133 |
| GPR150-Tango | 0 | −0.241 | 1.000 | 0.387 | −0.241 | 1.107 | 0.107 | 0.823 |
| GPR151-Tango | 0 | −0.076 | −0.144 | 0.410 | 0.311 | −0.426 | −0.426 | −0.191 |
| GPR152-Tango | 0 | 0.116 | −0.329 | −0.187 | −0.139 | −0.057 | 0.317 | 0.466 |
| GPR153-Tango | 0 | 0.011 | 0.042 | −0.891 | 0.405 | 0.286 | 0.213 | −0.024 |
| GPR156-Tango | 0 | 0.890 | 1.129 | 1.355 | 1.000 | 0.809 | 0.224 | 0.737 |
| GPR157-Tango | 0 | −0.039 | 0.384 | −0.640 | 1.147 | 0.508 | 0.082 | 0.541 |
| GPR158-Tango | 0 | −0.102 | 0.168 | 0.010 | −0.295 | 0.096 | −0.040 | 0.068 |
| GPR160-Tango | 0 | 0.351 | 0.261 | −0.041 | 0.116 | 0.260 | −0.203 | −0.071 |
| GPR161-Tango | 0 | 0.022 | −0.129 | 0.100 | 0.344 | 0.273 | −0.195 | −0.207 |
| GPR162-Tango | 0 | −0.140 | 0.193 | −0.140 | 0.819 | 0.059 | −0.685 | 0.059 |
| GPR17-Tango | 0 | 0.111 | 0.127 | −0.352 | 0.404 | 0.342 | 0.008 | −0.006 |
| GPR171-Tango | 0 | 0.564 | 0.655 | −0.520 | −0.097 | 0.044 | 0.090 | 0.395 |
| GPR173-Tango | 0 | 1.246 | 0.381 | −0.104 | 1.407 | 0.783 | 0.528 | 0.381 |
| GPR174-Tango | 0 | 0.387 | 0.300 | −0.136 | 0.072 | −0.331 | 0.429 | 0.300 |
| GPR18-Tango | 0 | 0.273 | 0.808 | −0.450 | 0.637 | 0.553 | 0.273 | 0.839 |
| GPR182-Tango | 0 | 0.508 | 0.447 | 0.613 | 0.690 | 0.399 | 0.020 | −0.290 |
| GPR183-Tango | 0 | 0.340 | 0.114 | −0.297 | 0.095 | −0.080 | −0.231 | −0.161 |
| GPR19-Tango | 0 | 0.440 | 0.475 | 0.137 | −0.006 | 0.010 | −0.092 | 0.302 |
| GPR20-Tango | 0 | −0.801 | −0.170 | −1.231 | −0.329 | −0.801 | −0.545 | −0.082 |
| GPR21-Tango | 0 | 1.000 | 0.888 | 0.888 | 0.632 | −0.515 | −1.737 | 1.263 |
| GPR22-Tango | 0 | 0.422 | 0.414 | 0.458 | 0.313 | 0.590 | 0.070 | 0.060 |
| GPR25-Tango | 0 | 1.112 | 1.697 | 1.112 | 1.217 | −0.122 | 0.491 | 1.697 |
| GPR26-Tango | 0 | 0.868 | 0.846 | 0.519 | 0.791 | 0.742 | −0.022 | 0.611 |
| GPR27-Tango | 0 | −1.369 | −1.369 | −2.369 | −0.147 | −0.784 | −0.147 | −0.495 |
| GPR3-Tango | 0 | 0.361 | 0.368 | 0.521 | 1.234 | 0.895 | −0.131 | −0.017 |
| GPR31-Tango | 0 | 0.419 | 0.386 | 0.543 | 0.978 | 0.747 | −0.129 | −0.370 |
| GPR32-Tango | 0 | 1.007 | 0.732 | −0.486 | 2.107 | 1.519 | 1.052 | 1.308 |
| GPR34-Tango | 0 | 0.255 | 0.476 | 0.787 | 1.255 | 1.393 | 0.850 | 0.476 |
| GPR35-Tango | 0 | 0.445 | 0.413 | −0.296 | 1.177 | 0.752 | 0.404 | 0.396 |
| GPR37-Tango | 0 | 0.468 | 0.491 | 0.068 | 1.221 | 1.132 | 0.243 | 0.503 |
| GPR37L1-Tango | 0 | 0.474 | 0.454 | −0.287 | 0.290 | 0.575 | 0.403 | 0.593 |
| GPR39-Tango | 0 | 0.087 | 0.629 | 0.267 | −0.328 | 0.087 | 0.150 | 0.022 |
| GPR4-Tango | 0 | 0.160 | 0.138 | 0.352 | 0.586 | 0.379 | −0.155 | 0.190 |
| GPR44-Tango | 0 | 0.609 | 0.544 | 0.205 | 1.577 | 0.702 | 0.790 | 0.609 |
| GPR45-Tango | 0 | 0.190 | 0.455 | −1.400 | 0.982 | 0.236 | −0.407 | 0.331 |
| GPR50-Tango | 0 | 0.553 | 0.620 | 0.125 | 0.813 | 0.093 | 0.291 | 0.345 |
| GPR52-Tango | 0 | 0.885 | 0.796 | 0.338 | 0.830 | 0.830 | −0.148 | 0.188 |
| GPR55-Tango | 0 | −0.049 | 0.130 | −0.227 | 0.408 | 0.021 | −0.117 | 0.052 |
| GPR56-Tango | 0 | 0.606 | 0.612 | 0.825 | 0.772 | 0.433 | −0.209 | 0.411 |
| GPR6-Tango | 0 | 0.825 | 0.989 | 1.181 | 1.443 | 1.158 | −0.092 | −0.037 |
| GPR61-Tango | 0 | −0.557 | −0.187 | −0.164 | −0.426 | −0.383 | −0.784 | −0.459 |
| GPR62-Tango | 0 | 0.485 | −0.152 | 0.485 | 0.485 | 0.485 | −0.152 | 0.138 |
| GPR63-Tango | 0 | 0.170 | 0.187 | −1.100 | 0.254 | 0.280 | 0.008 | −0.058 |
| GPR64-Tango | 0 | −0.151 | −0.134 | 0.407 | 0.057 | −0.003 | −0.281 | −0.328 |
| GPR65-Tango | 0 | −0.065 | −0.080 | −0.040 | −0.198 | −0.210 | −0.009 | −0.157 |
| GPR68-Tango | 0 | 0.026 | 0.068 | −0.544 | −0.519 | −0.638 | 0.166 | −0.150 |
| GPR75- Tango | 0 | 0.107 | 0.132 | −0.281 | 0.294 | 0.076 | −0.872 | 0.105 |
| GPR77-Tango | 0 | 0.595 | 0.380 | 0.681 | 0.738 | 0.943 | −0.587 | 0.322 |
| GPR78-Tango | 0 | −0.187 | 0.282 | 0.245 | 0.442 | 0.357 | −0.257 | 0.018 |
| GPR82-Tango | 0 | 0.310 | 0.068 | 0.638 | 1.036 | 0.842 | −0.360 | −0.284 |
| GPR83-Tango | 0 | 0.011 | 0.079 | −1.500 | −0.006 | 0.095 | 0.320 | 0.253 |
| GPR84-Tango | 0 | 0.321 | 0.140 | 0.030 | 0.561 | 0.543 | 0.042 | 0.099 |
| GPR85-Tango | 0 | 0.459 | 0.551 | −0.163 | 0.677 | 0.722 | 0.311 | 0.336 |
| GPR87-Tango | 0 | 0.049 | 0.131 | −0.482 | 0.805 | 0.634 | 0.109 | 0.223 |
| GPR88-Tango | 0 | −0.163 | 0.252 | 0.218 | 0.636 | 0.339 | −0.915 | −0.391 |
| GPR97-Tango | 0 | 1.023 | 1.149 | 1.521 | 1.564 | 1.127 | −0.016 | 0.191 |
| GPRC5A-Tango | 0 | −0.206 | −0.144 | −0.047 | 0.093 | −0.124 | 0.011 | 0.075 |
| GPRC5B-Tango | 0 | 0.045 | 0.203 | −0.143 | 0.311 | 0.328 | −0.016 | −0.348 |
| GPRC5C-Tango | 0 | 0.265 | 0.047 | −1.061 | −0.158 | 0.054 | −0.153 | 0.023 |
| GPRC5D-Tango | 0 | 0.413 | 0.472 | −0.665 | 0.369 | 0.277 | 0.071 | 0.273 |
| GPRC6A-Tango | 0 | −0.048 | −0.064 | −2.030 | −0.245 | −0.118 | 0.044 | −0.233 |
| GRM7-Tango | 0 | −0.202 | −0.231 | −0.957 | −0.213 | −0.261 | −0.528 | −0.240 |
| GRM8-Tango | 0 | −0.113 | −0.321 | −0.333 | −0.122 | −0.109 | −0.129 | −0.125 |
| GRPR-Tango | 0 | 0.013 | −0.308 | −1.919 | −0.200 | 0.079 | −0.394 | −0.269 |

| PRESTO-Tango | Faecalibacterium | Oscillospira | Oscillospira | Oscillospira | Lactobacillus | Lactobacillus | Lactobacillus | Lactobacillus |
|---|---|---|---|---|---|---|---|---|
| with Gifu Medium | prausnitzii | spp-1# | spp-2# | spp-3# | reuteri-1# | reuteri-2# | reuteri-3# | UC |
| GPR1-Tango | −0.284 | −0.022 | −0.559 | −1.170 | 0.123 | 0.361 | 0.107 | −0.241 |
| GPR101-Tango | −0.102 | −0.045 | −0.045 | −0.349 | 0.159 | 0.074 | 0.074 | 0.268 |
| GPR110-Tango | −0.272 | −0.279 | −0.563 | −0.587 | 0.224 | −0.226 | 0.112 | −0.011 |
| GPR111-Tango | 0.485 | 1.297 | 0.436 | 0.543 | 0.695 | 0.809 | 0.216 | 0.308 |
| GPR113-Tango | −0.172 | −0.007 | −0.242 | −0.686 | 0.487 | −0.149 | −0.172 | −0.258 |
| GPR114-Tango | 0.165 | 0.580 | −0.016 | 0.247 | −0.298 | 0.325 | −0.700 | 0.077 |
| GPR115-Tango | 0.017 | −0.290 | −0.413 | −0.524 | −0.056 | −0.123 | −0.097 | −0.120 |
| GPR116-Tango | −0.072 | 0.259 | −0.317 | −0.515 | 0.265 | 0.165 | 0.134 | 0.070 |
| GPR119-Tango | 0.263 | 1.287 | 0.660 | 0.864 | 0.986 | 0.623 | 0.444 | 0.506 |
| GPR12-Tango | −0.083 | −0.215 | −0.099 | −0.330 | −0.367 | −0.417 | −0.156 | −0.628 |
| GPR120-Tango | 0.112 | 1.217 | 1.282 | −0.402 | 1.000 | 0.491 | 0.314 | −0.122 |
| GPR123-Tango | −0.294 | −0.211 | −1.015 | −0.633 | 0.015 | −0.051 | −0.294 | −0.316 |
| GPR124-Tango | 0.165 | 0.547 | 0.206 | 0.580 | 0.000 | 0.750 | 0.122 | 0.672 |
| GPR125-Tango | −0.029 | −0.344 | −0.335 | −0.628 | −0.203 | −0.163 | −0.111 | −0.388 |
| GPR126-Tango | 0.259 | 0.081 | −0.256 | −0.751 | −0.349 | 0.126 | 0.024 | 0.725 |
| GPR132-Tango | −0.168 | −0.490 | −0.960 | −1.133 | 0.050 | 0.170 | 0.204 | −0.192 |
| GPR133-Tango | −0.045 | −0.347 | −0.235 | −0.594 | −0.050 | 0.024 | −0.008 | −0.097 |
| GPR135-Tango | −1.011 | −1.733 | −1.885 | −1.231 | −1.181 | −1.490 | −0.820 | −0.585 |
| GPR141-Tango | −0.104 | −0.636 | −0.449 | −0.571 | −0.825 | −0.354 | −0.199 | −0.508 |
| GPR142-Tango | 0.472 | 0.415 | 0.160 | 0.960 | −0.472 | 0.228 | 0.835 | 0.415 |
| GPR143-Tango | −0.146 | −0.004 | −0.534 | −0.664 | 0.615 | 0.250 | 0.140 | −0.136 |
| GPR144-Tango | −0.006 | 0.229 | −0.617 | 0.173 | −0.154 | 0.373 | −0.072 | −0.367 |
| GPR146-Tango | 0.485 | −0.515 | −0.234 | −0.515 | 1.170 | −0.234 | 0.202 | 0.379 |
| GPR148-Tango | −0.339 | 0.528 | 0.783 | −0.104 | 0.097 | 0.274 | 0.528 | 0.783 |
| GPR149-Tango | −0.157 | 1.213 | 0.336 | 0.771 | 0.791 | 0.637 | 0.203 | 0.899 |
| GPR15-Tango | −1.012 | −0.878 | −1.878 | −0.816 | −0.381 | −0.608 | −0.381 | −0.293 |
| GPR150-Tango | 1.107 | −0.241 | 0.823 | 0.823 | 0.387 | −1.115 | 0.387 | 0.621 |
| GPR151-Tango | −0.513 | −0.144 | −0.191 | −0.265 | 0.206 | −0.672 | −0.144 | 0.531 |
| GPR152-Tango | −0.259 | 0.735 | 0.356 | 0.174 | 0.344 | 0.247 | 0.293 | 0.123 |
| GPR153-Tango | 0.096 | −0.110 | −0.328 | −0.501 | −0.070 | 0.379 | 0.122 | −0.048 |
| GPR156-Tango | 0.525 | 0.779 | 0.674 | −0.048 | 0.694 | 0.768 | 0.539 | 0.394 |
| GPR157-Tango | −0.459 | 0.811 | −0.087 | 0.167 | −0.337 | 0.126 | −0.138 | 0.082 |
| GPR158-Tango | −0.635 | 0.294 | −0.517 | −1.040 | −0.259 | 0.068 | 0.294 | −0.071 |
| GPR160-Tango | 0.025 | −0.411 | −1.085 | −1.008 | 0.210 | 0.264 | 0.186 | −0.255 |
| GPR161-Tango | −0.100 | −0.839 | −0.959 | −1.130 | −0.280 | −0.093 | −0.067 | −0.338 |
| GPR162-Tango | −0.685 | −0.807 | −1.807 | −1.573 | −0.310 | −0.469 | −0.573 | 0.012 |
| GPR17-Tango | −0.078 | −0.028 | 0.061 | −0.456 | 0.118 | 0.160 | 0.038 | −0.054 |
| GPR171-Tango | −0.077 | 0.631 | −0.275 | −0.027 | 0.740 | 0.600 | 0.141 | −0.253 |
| GPR173-Tango | −0.339 | 1.066 | 0.097 | −0.339 | −0.339 | −0.104 | 0.097 | 0.528 |
| GPR174-Tango | 0.269 | 0.469 | −0.613 | 0.018 | 0.174 | 0.222 | −0.453 | −0.331 |
| GPR18-Tango | 0.171 | 0.678 | 0.126 | 0.292 | 0.470 | 0.139 | 0.207 | −0.280 |
| GPR182-Tango | −0.316 | −0.187 | −0.191 | −0.667 | −0.063 | −0.311 | −0.316 | −0.116 |
| GPR183-Tango | −0.911 | −0.446 | −0.937 | −0.184 | −0.231 | 0.076 | 0.017 | −0.272 |
| GPR19-Tango | 0.234 | −0.790 | −1.294 | −1.186 | 0.391 | 0.274 | 0.310 | −0.065 |
| GPR20-Tango | 0.078 | 0.267 | −0.801 | −0.948 | −1.433 | −0.433 | −0.801 | −0.329 |
| GPR21-Tango | 0.766 | 1.511 | 1.000 | 1.350 | 1.433 | 0.379 | 0.632 | 1.104 |
| GPR22-Tango | 0.142 | −0.201 | −0.424 | −0.537 | 0.060 | 0.255 | −0.247 | 0.152 |
| GPR25-Tango | −0.122 | 0.745 | 0.112 | 0.878 | 0.598 | 1.000 | 1.463 | 0.314 |
| GPR26-Tango | 0.235 | 0.640 | 0.550 | 0.198 | 0.658 | 0.566 | 0.595 | 0.725 |
| GPR27-Tango | −0.147 | −0.495 | 0.133 | 0.368 | −0.495 | 0.746 | 0.368 | −1.369 |
| GPR3-Tango | 0.120 | −0.340 | −0.264 | −0.774 | −0.170 | −0.295 | −0.136 | −0.136 |
| GPR31-Tango | 0.258 | −0.614 | −1.549 | −0.370 | −0.549 | −0.730 | −1.052 | 0.036 |
| GPR32-Tango | 0.708 | 0.862 | −0.312 | 0.189 | 1.114 | 1.026 | 0.997 | 0.720 |
| GPR34-Tango | 0.365 | 1.255 | 0.286 | 0.962 | 0.728 | 0.943 | 0.829 | 0.683 |
| GPR35-Tango | 0.330 | 0.132 | −0.271 | −0.285 | 0.663 | 0.787 | 0.436 | 0.261 |
| GPR37-Tango | 0.415 | 0.184 | 0.245 | 0.006 | 0.187 | 0.414 | 0.296 | 0.173 |
| GPR37L1-Tango | 0.432 | 0.319 | −0.326 | −0.222 | 0.626 | 0.607 | 0.552 | 0.277 |
| GPR39-Tango | −0.046 | −0.093 | −0.093 | 0.585 | −0.093 | −0.167 | −0.046 | −0.328 |
| GPR4-Tango | 0.213 | −0.667 | −0.617 | −0.845 | −0.312 | −0.038 | 0.021 | −0.070 |
| GPR44-Tango | 0.790 | 1.421 | 0.205 | −0.561 | −0.182 | 0.609 | −0.075 | 0.609 |
| GPR45-Tango | 0.117 | 0.023 | −0.835 | −0.867 | 0.381 | 0.117 | 0.040 | −0.470 |
| GPR50-Tango | 0.263 | 0.966 | −0.200 | −0.487 | 0.774 | 0.635 | 0.291 | 0.319 |
| GPR52-Tango | −0.082 | 0.040 | −0.744 | 0.134 | 0.338 | 0.097 | −0.263 | 0.624 |
| GPR55-Tango | −0.081 | −0.216 | −0.322 | −0.766 | −0.089 | 0.458 | 0.094 | −0.216 |
| GPR56-Tango | 0.149 | −0.220 | −0.508 | −0.592 | 0.375 | 0.230 | 0.259 | 0.661 |
| GPR6-Tango | 0.287 | 0.026 | −0.272 | −0.473 | −0.072 | 0.114 | 0.054 | −0.224 |
| GPR61-Tango | −0.862 | −0.754 | −1.299 | −0.992 | −0.215 | −0.643 | −0.773 | −0.537 |
| GPR62-Tango | 1.202 | 0.766 | 1.202 | 1.379 | 0.766 | 0.485 | −0.737 | −0.152 |
| GPR63-Tango | 0.104 | −0.127 | −0.001 | −0.168 | 0.113 | 0.135 | 0.158 | −0.229 |
| GPR64-Tango | −0.182 | −0.095 | −0.261 | −0.419 | 0.083 | −0.085 | 0.013 | −0.343 |
| GPR65-Tango | −0.076 | −0.413 | −0.440 | −0.608 | −0.204 | −0.145 | −0.252 | −0.152 |
| GPR68-Tango | −0.406 | −0.599 | −1.205 | −0.838 | 0.073 | −0.083 | −0.044 | −0.321 |
| GPR75- Tango | −0.399 | −0.636 | −1.242 | −1.074 | 0.057 | −0.016 | −0.151 | −0.293 |
| GPR77-Tango | 0.022 | −0.736 | −1.021 | −1.030 | 0.025 | 0.127 | 0.008 | −0.184 |
| GPR78-Tango | −0.150 | −0.678 | −0.475 | −0.939 | −0.491 | −0.228 | −0.609 | 0.103 |
| GPR82-Tango | 0.011 | −0.478 | −0.901 | −0.814 | −0.535 | −0.129 | −0.348 | −0.334 |
| GPR83-Tango | −0.171 | 0.009 | −0.720 | −0.651 | 0.301 | 0.187 | 0.262 | −0.190 |
| GPR84-Tango | 0.067 | −0.313 | −0.470 | −0.568 | 0.152 | 0.214 | 0.067 | 0.059 |
| GPR85-Tango | 0.160 | 0.251 | −0.363 | −0.564 | 0.224 | 0.405 | 0.311 | 0.023 |
| GPR87-Tango | −0.010 | −0.104 | −0.165 | −0.234 | 0.007 | 0.186 | 0.126 | −0.106 |
| GPR88-Tango | −0.391 | −0.726 | −0.726 | −1.539 | −0.391 | −1.322 | −0.163 | −0.539 |
| GPR97-Tango | −0.064 | 0.407 | 0.031 | −0.569 | 0.149 | 0.442 | 0.532 | 0.499 |
| GPRC5A-Tango | −0.583 | −0.483 | −0.861 | −1.118 | −0.202 | −0.121 | −0.090 | −0.159 |
| GPRC5B-Tango | −0.097 | −0.741 | −1.073 | −1.051 | 0.110 | 0.040 | 0.176 | −0.354 |
| GPRC5C-Tango | −0.047 | −0.044 | −0.240 | −0.318 | −0.082 | −0.053 | −0.030 | −0.286 |
| GPRC5D-Tango | 0.174 | −0.075 | −0.361 | −0.533 | 0.413 | 0.453 | 0.267 | −0.422 |
| GPRC6A-Tango | −0.126 | −0.493 | −0.673 | −0.851 | −0.118 | −0.057 | −0.071 | −0.315 |
| GRM7-Tango | −0.178 | −0.613 | −0.786 | −0.790 | −0.059 | −0.140 | −0.242 | −0.528 |
| GRM8-Tango | −0.386 | −0.177 | −0.671 | −0.914 | 0.046 | 0.156 | −0.162 | −0.359 |
| GRPR-Tango | 0.256 | 0.234 | −0.736 | 0.241 | 0.557 | 0.761 | 0.487 | −0.065 |

| PRESTO-Tango | (Clostridiales) | Pediococcus | Pediococcus | Pediococcus | Streptococcus | Streptococcus | Streptococcus | Streptococcus |
|---|---|---|---|---|---|---|---|---|
| with Gifu Medium | UC/UC | UC-1# | UC-2# | UC-3# | luteciae | spp-1# | spp-2# | spp-3# |
| GPR1-Tango | −0.446 | −0.439 | −0.487 | −0.439 | 0.297 | 0.588 | −0.125 | 0.552 |
| GPR101-Tango | 0.039 | −0.004 | 0.499 | 0.130 | 0.234 | 0.725 | 0.422 | 1.613 |
| GPR110-Tango | 0.167 | 0.112 | −0.100 | 0.194 | 0.499 | 0.775 | 0.020 | 1.932 |
| GPR111-Tango | 1.753 | −0.160 | 0.466 | 0.626 | −0.357 | 0.543 | 0.669 | −0.357 |
| GPR113-Tango | 0.046 | −0.195 | 0.104 | −0.090 | −0.402 | 0.085 | −0.384 | −1.157 |
| GPR114-Tango | 0.165 | −0.807 | −0.016 | −0.298 | 0.077 | 0.515 | 0.077 | −0.807 |
| GPR115-Tango | −0.196 | −0.241 | −0.154 | −0.129 | −0.125 | 0.121 | −0.024 | −0.758 |
| GPR116-Tango | 0.082 | 0.009 | 0.542 | 0.185 | 0.157 | 0.695 | 0.295 | −0.230 |
| GPR119-Tango | 0.566 | −0.434 | −0.837 | 0.660 | 1.111 | 0.189 | 1.322 | 0.566 |
| GPR12-Tango | −0.312 | −0.986 | 0.429 | −0.039 | −0.348 | −0.571 | −0.886 | 0.798 |
| GPR120-Tango | −0.402 | 0.745 | 0.598 | 0.112 | −0.402 | 1.112 | 1.545 | −0.122 |
| GPR123-Tango | 0.214 | −0.606 | −0.606 | −0.033 | 0.654 | 0.706 | −0.746 | 1.975 |
| GPR124-Tango | 0.077 | 0.458 | 0.165 | 0.000 | −0.241 | 1.697 | 0.312 | 3.000 |
| GPR125-Tango | −0.246 | −0.158 | −0.198 | −0.232 | −0.035 | 0.237 | 0.062 | 0.014 |
| GPR126-Tango | −0.256 | 0.208 | −0.564 | 0.112 | 0.346 | 1.978 | −0.103 | 2.965 |
| GPR132-Tango | −0.533 | −0.027 | −0.570 | 0.035 | 0.066 | −0.203 | −0.312 | −0.513 |
| GPR133-Tango | −0.104 | −0.043 | 0.059 | 0.120 | −0.068 | −0.023 | 0.137 | −0.486 |
| GPR135-Tango | −0.885 | −0.846 | −0.674 | −0.206 | −0.300 | −0.411 | −0.585 | −0.283 |
| GPR141-Tango | −0.249 | −1.166 | 0.162 | −0.249 | −0.449 | −0.903 | −0.354 | 2.270 |
| GPR142-Tango | −0.368 | 0.293 | 0.293 | 0.509 | 0.760 | 1.325 | 0.665 | 1.954 |
| GPR143-Tango | 0.163 | −0.023 | −0.508 | 0.325 | 0.391 | −0.102 | 0.067 | −1.379 |
| GPR144-Tango | −0.006 | −0.367 | −0.006 | −0.367 | −0.112 | −0.006 | 0.196 | 1.445 |
| GPR146-Tango | 0.485 | −0.515 | −0.862 | 0.632 | 0.000 | 1.000 | −1.152 | −0.234 |
| GPR148-Tango | 0.896 | −1.841 | −0.104 | 1.246 | 0.097 | 0.097 | −0.619 | −1.256 |
| GPR149-Tango | 0.280 | −0.305 | 0.737 | 0.652 | 0.849 | 1.198 | 1.017 | 1.604 |
| GPR15-Tango | −0.943 | −1.878 | −1.012 | −0.878 | −0.171 | −0.756 | −1.540 | 1.409 |
| GPR150-Tango | 1.000 | −0.241 | 0.107 | 1.107 | 0.387 | 1.107 | 0.107 | 1.387 |
| GPR151-Tango | 0.052 | −0.191 | 0.111 | 0.111 | −0.265 | 0.797 | −0.144 | 1.833 |
| GPR152-Tango | 0.435 | 0.116 | −0.148 | 0.472 | 0.028 | −0.021 | 0.192 | −0.078 |
| GPR153-Tango | 0.084 | −0.177 | 0.459 | 0.035 | 0.127 | −0.063 | −0.043 | −0.925 |
| GPR156-Tango | 0.565 | −0.158 | 0.490 | 0.690 | 0.586 | 1.906 | 0.752 | 2.074 |
| GPR157-Tango | 0.682 | −1.055 | 0.682 | −0.280 | −0.189 | 0.541 | −0.337 | −0.243 |
| GPR158-Tango | 0.545 | −0.476 | −0.370 | 0.039 | 0.245 | 1.314 | −0.190 | 2.020 |
| GPR160-Tango | 0.020 | −0.207 | −0.461 | −0.092 | 0.086 | 0.287 | −0.144 | 0.832 |
| GPR161-Tango | −0.847 | −0.188 | −0.242 | −0.461 | −0.128 | 0.764 | −0.479 | 1.147 |
| GPR162-Tango | −0.062 | −0.573 | −0.685 | −0.469 | 0.128 | −0.807 | −0.685 | 0.128 |
| GPR17-Tango | −0.011 | −0.002 | 0.099 | −0.032 | 0.194 | 0.269 | 0.033 | −0.563 |
| GPR171-Tango | 0.677 | −0.849 | −0.190 | 0.807 | 0.564 | 0.168 | 0.027 | −0.420 |
| GPR173-Tango | 0.661 | −0.339 | 0.274 | 0.783 | 0.097 | 0.661 | −0.104 | −0.619 |
| GPR174-Tango | 0.645 | −0.076 | 0.072 | −0.136 | 0.573 | 0.429 | 0.072 | −0.331 |
| GPR18-Tango | 0.894 | −0.874 | −0.119 | 0.628 | 0.669 | 0.678 | 0.171 | −0.874 |
| GPR182-Tango | 0.128 | −0.149 | 0.966 | −0.218 | 0.149 | 0.263 | 0.137 | 0.963 |
| GPR183-Tango | 0.263 | −0.873 | 0.163 | −0.348 | −0.102 | −0.297 | −1.017 | 0.631 |
| GPR19-Tango | −0.063 | 0.149 | −0.484 | 0.102 | 0.289 | 0.335 | −0.090 | 1.119 |
| GPR20-Tango | 0.199 | −1.231 | 0.000 | 0.333 | 0.000 | −0.329 | 0.078 | −0.948 |
| GPR21-Tango | 1.982 | 0.485 | −0.515 | 0.632 | 1.433 | −0.234 | 0.379 | 1.000 |
| GPR22-Tango | 0.180 | −0.037 | −0.247 | 0.080 | 0.770 | 1.031 | −0.226 | 1.885 |
| GPR25-Tango | 0.598 | 0.598 | 0.491 | 1.745 | 1.376 | −0.122 | 1.000 | 1.000 |
| GPR26-Tango | 0.734 | 0.150 | 0.530 | 0.884 | 0.630 | 0.782 | 0.323 | 0.150 |
| GPR27-Tango | −0.784 | −1.369 | −0.147 | −0.495 | 0.133 | 0.133 | −0.147 | 0.746 |
| GPR3-Tango | −0.222 | −0.656 | 0.818 | −0.080 | −0.247 | −0.097 | −0.109 | 1.086 |
| GPR31-Tango | 0.158 | −1.145 | 0.294 | −0.333 | 0.036 | −0.427 | −0.487 | 2.699 |
| GPR32-Tango | 0.732 | 0.273 | 0.638 | 1.657 | 0.828 | 0.074 | 0.684 | −0.806 |
| GPR34-Tango | 0.545 | −0.279 | 0.620 | 0.765 | −0.842 | 0.808 | 0.316 | 0.335 |
| GPR35-Tango | 0.127 | 0.037 | 0.088 | 0.706 | 0.341 | 0.139 | 0.014 | −0.564 |
| GPR37-Tango | 0.407 | 0.221 | 0.643 | 0.455 | 0.167 | 0.471 | 0.456 | −0.062 |
| GPR37L1-Tango | 0.316 | 0.261 | 0.391 | 0.547 | 0.555 | 0.387 | 0.590 | −0.431 |
| GPR39-Tango | 0.304 | 0.022 | −0.245 | 0.022 | 0.409 | 0.555 | 0.022 | 1.229 |
| GPR4-Tango | −0.110 | −0.205 | −0.030 | 0.371 | 0.259 | 0.405 | 0.239 | 1.191 |
| GPR44-Tango | 0.872 | −0.561 | 0.072 | 0.609 | 1.672 | 0.790 | −0.561 | 2.925 |
| GPR45-Tango | −0.282 | −0.614 | −0.147 | 0.190 | 0.190 | −0.282 | 0.317 | −2.199 |
| GPR50-Tango | 0.505 | −0.585 | −0.487 | 0.813 | 0.851 | −0.064 | 0.489 | 0.319 |
| GPR52-Tango | −0.337 | −0.263 | −0.082 | −0.148 | 0.290 | −0.263 | −0.848 | 1.874 |
| GPR55-Tango | 0.181 | −0.044 | −0.202 | 0.253 | 0.352 | 0.495 | 0.145 | 0.114 |
| GPR56-Tango | 0.205 | −0.016 | −0.123 | 0.256 | 0.291 | 1.235 | 0.418 | 1.732 |
| GPR6-Tango | 0.472 | −0.211 | 1.190 | −0.026 | 0.696 | 0.683 | 0.553 | 1.371 |
| GPR61-Tango | −0.199 | −0.932 | −0.613 | −0.754 | −0.493 | −0.172 | −0.819 | 0.733 |
| GPR62-Tango | 0.138 | 0.138 | 0.485 | −1.737 | 0.485 | 1.379 | 1.000 | 1.379 |
| GPR63-Tango | −0.112 | −0.342 | 0.385 | −0.045 | −0.168 | 0.323 | 0.262 | −1.169 |
| GPR64-Tango | −0.229 | −0.274 | −0.125 | −0.167 | −0.316 | 0.113 | −0.035 | −0.215 |
| GPR65-Tango | −0.357 | −0.095 | −0.327 | −0.092 | 0.019 | 0.211 | −0.081 | 0.448 |
| GPR68-Tango | −0.195 | −0.206 | −0.625 | 0.205 | 0.185 | −0.144 | −0.173 | −0.715 |
| GPR75- Tango | −0.656 | −1.039 | −0.900 | −0.171 | 0.102 | 0.088 | −0.401 | 0.804 |
| GPR77-Tango | −0.354 | −0.627 | −0.044 | 0.103 | −0.178 | 0.163 | −0.469 | 1.288 |
| GPR78-Tango | −0.064 | −0.414 | −0.537 | −0.311 | −0.281 | −0.414 | −0.475 | 1.312 |
| GPR82-Tango | −0.185 | −0.549 | 1.050 | −0.159 | −0.268 | −0.318 | −0.224 | 1.545 |
| GPR83-Tango | 0.079 | −0.157 | −0.202 | 0.239 | 0.028 | −0.149 | −0.176 | −1.751 |
| GPR84-Tango | 0.187 | 0.071 | 0.100 | 0.074 | 0.220 | 0.452 | 0.332 | 0.000 |
| GPR85-Tango | −0.253 | −0.418 | 0.323 | 0.731 | 0.393 | 0.843 | 0.459 | −0.008 |
| GPR87-Tango | 0.084 | −0.065 | 0.169 | 0.158 | 0.078 | 0.261 | −0.011 | −0.636 |
| GPR88-Tango | −0.794 | −2.225 | −0.339 | −0.794 | −0.240 | −0.794 | −0.726 | 0.799 |
| GPR97-Tango | −0.257 | −0.114 | 0.231 | 0.595 | 0.407 | 1.303 | 0.532 | 2.905 |
| GPRC5A-Tango | −0.452 | −0.142 | −0.500 | −0.235 | 0.078 | 0.759 | −0.030 | 1.279 |
| GPRC5B-Tango | −0.278 | −0.525 | −0.146 | −0.642 | 0.176 | 0.620 | −0.217 | 1.382 |
| GPRC5C-Tango | 0.053 | −0.520 | −0.267 | 0.160 | 0.099 | 0.067 | −0.133 | −1.202 |
| GPRC5D-Tango | −0.048 | −0.215 | 0.064 | 0.388 | 0.424 | 0.363 | 0.158 | −0.543 |
| GPRC6A-Tango | −0.400 | −0.164 | −0.291 | −0.101 | −0.037 | −0.333 | 0.019 | −1.629 |
| GRM7-Tango | −0.438 | −0.432 | −0.479 | −0.202 | −0.288 | −0.194 | −0.372 | −1.321 |
| GRM8-Tango | −0.399 | −0.194 | −0.363 | 0.037 | 0.064 | 0.388 | −0.180 | −0.371 |
| GRPR-Tango | 0.256 | −0.544 | −0.993 | 0.445 | −0.200 | 0.062 | −0.230 | −2.356 |

| | Acidamino- | Acidamino- | Acidamino- | Acidamino- | | | | |
|---|---|---|---|---|---|---|---|---|
| PRESTO-Tango | coccus | coccus | coccus | coccus | Megasphaera | Megasphaera | Peptoniphilus | Peptoniphilus |
| with Gifu Medium | spp-1# | spp-2# | spp-3# | spp-4# | spp-1# | spp-2# | spp-1# | spp-2# |
| GPR1-Tango | −0.301 | 0.091 | 0.152 | −0.027 | −0.739 | −0.660 | 0.148 | −0.145 |
| GPR101-Tango | 0.045 | 0.261 | 0.274 | 0.189 | 0.164 | 0.093 | 0.242 | 0.164 |
| GPR110-Tango | 0.009 | 0.035 | 0.267 | 0.229 | −0.434 | 0.271 | 0.009 | 0.224 |
| GPR111-Tango | 0.770 | 1.420 | 1.357 | 0.937 | 0.643 | 1.430 | 0.958 | 1.181 |
| GPR113-Tango | 0.066 | 0.153 | 0.516 | 0.303 | 0.558 | 0.346 | 0.607 | 0.189 |
| GPR114-Tango | −0.016 | 0.247 | 0.247 | 0.077 | 0.077 | 0.952 | 0.077 | −0.016 |
| GPR115-Tango | −0.203 | −0.180 | −0.146 | −0.145 | −0.323 | −0.168 | −0.313 | −0.151 |
| GPR116-Tango | 0.338 | 0.234 | 0.264 | 0.345 | −0.131 | 0.234 | 0.317 | 0.282 |
| GPR119-Tango | 0.766 | 1.356 | 0.941 | 1.251 | 0.029 | 1.411 | 1.176 | 0.766 |
| GPR12-Tango | −1.063 | −0.436 | 0.065 | −0.312 | −0.014 | −0.199 | −0.199 | −0.109 |
| GPR120-Tango | −0.122 | −1.040 | 0.491 | −0.122 | 1.282 | 1.217 | 0.598 | 0.314 |
| GPR123-Tango | −0.353 | 0.110 | 0.126 | 0.298 | −0.858 | 0.786 | −0.316 | 0.032 |
| GPR124-Tango | 0.580 | −0.601 | 0.547 | 0.515 | 0.832 | 0.721 | 1.016 | 1.016 |
| GPR125-Tango | −0.397 | −0.124 | −0.184 | −0.213 | −0.216 | −0.103 | −0.265 | −0.350 |
| GPR126-Tango | −0.103 | 0.056 | 0.272 | 0.126 | −0.069 | 0.495 | 0.096 | 0.371 |
| GPR132-Tango | −0.592 | −0.449 | −0.594 | −0.650 | −0.953 | −0.468 | −0.635 | −0.601 |
| GPR133-Tango | 0.100 | −0.208 | −0.073 | −0.086 | −0.542 | −0.366 | −0.132 | −0.234 |
| GPR135-Tango | −1.055 | −0.383 | −0.283 | −1.011 | −0.640 | −0.710 | −0.640 | −0.846 |
| GPR141-Tango | −0.986 | −0.150 | −0.704 | −0.073 | −0.903 | −0.508 | −0.508 | −0.104 |
| GPR142-Tango | −0.209 | 0.791 | 0.713 | 0.835 | 0.665 | 0.563 | 0.835 | 1.100 |
| GPR143-Tango | −0.219 | 0.052 | −0.036 | 0.055 | −0.620 | 0.008 | −0.048 | −0.129 |
| GPR144-Tango | 0.031 | 0.229 | 0.196 | 0.068 | 0.104 | 0.292 | 0.031 | 0.402 |
| GPR146-Tango | 0.379 | −0.234 | 0.632 | 0.766 | 0.485 | 1.000 | 0.632 | 0.202 |
| GPR148-Tango | 0.097 | 1.159 | 1.481 | 0.097 | 1.246 | 0.661 | −0.619 | 0.097 |
| GPR149-Tango | 0.252 | 1.331 | 1.252 | 1.266 | 2.306 | 1.784 | 1.167 | 1.167 |
| GPR15-Tango | −0.816 | −1.211 | −1.012 | −0.699 | −0.556 | −1.059 | −0.252 | −0.381 |
| GPR150-Tango | 0.107 | −0.241 | −0.241 | 0.621 | 1.387 | 0.107 | −0.531 | 0.387 |
| GPR151-Tango | 0.722 | 0.111 | 0.410 | 1.082 | 0.696 | 0.833 | 0.111 | 0.206 |
| GPR152-Tango | 0.389 | 0.404 | 0.389 | 0.617 | −0.611 | 0.258 | 0.323 | 0.415 |
| GPR153-Tango | −0.076 | 0.093 | −0.133 | −0.170 | −0.084 | −0.104 | −0.215 | −0.166 |
| GPR156-Tango | 0.079 | 0.272 | 0.698 | 0.662 | 0.596 | 0.677 | 0.748 | 0.775 |
| GPR157-Tango | 0.653 | 1.147 | 0.953 | 0.977 | 1.401 | 0.888 | 0.633 | 0.541 |
| GPR158-Tango | −0.010 | 0.787 | 0.294 | 0.245 | 0.010 | 0.294 | 0.096 | 0.194 |
| GPR160-Tango | −0.559 | −0.425 | −0.215 | −0.163 | −0.418 | −0.314 | −0.269 | −0.205 |
| GPR161-Tango | −0.588 | −0.489 | −0.422 | −0.563 | −0.357 | −0.521 | −0.441 | −0.405 |
| GPR162-Tango | −0.807 | 0.059 | −0.573 | −0.062 | 0.059 | −0.310 | −0.807 | −0.310 |
| GPR17-Tango | −0.208 | −0.024 | −0.048 | −0.036 | 0.271 | −0.102 | 0.005 | 0.048 |
| GPR171-Tango | −0.077 | 0.271 | 0.480 | 0.518 | 0.215 | 0.769 | 0.480 | 0.729 |
| GPR173-Tango | 0.896 | 1.066 | 0.097 | 0.783 | 0.661 | 0.274 | 0.783 | 0.274 |
| GPR174-Tango | 0.174 | 0.834 | 0.802 | 0.943 | 0.300 | 0.770 | 0.737 | 0.609 |
| GPR18-Tango | 0.014 | 0.808 | 1.250 | 1.120 | 1.020 | 0.824 | 0.694 | 0.655 |
| GPR182-Tango | 0.083 | 0.156 | 0.194 | 0.146 | 0.218 | −0.142 | 0.123 | 0.037 |
| GPR183-Tango | −0.374 | 0.127 | 0.217 | 0.372 | −0.080 | −0.102 | −0.161 | −0.117 |
| GPR19-Tango | −0.366 | −0.144 | 0.129 | −0.045 | −0.619 | −0.061 | −0.205 | −0.123 |
| GPR20-Tango | 0.267 | −0.082 | 0.395 | 0.918 | −0.667 | 0.152 | 0.199 | 0.333 |
| GPR21-Tango | 1.104 | 1.350 | 0.379 | 1.104 | 1.170 | 1.982 | 1.511 | 1.945 |
| GPR22-Tango | 0.214 | −0.177 | 0.296 | 0.385 | 0.536 | 0.430 | 0.107 | 0.369 |
| GPR25-Tango | 0.491 | 1.000 | 1.376 | 0.745 | 0.878 | 1.745 | 1.000 | 1.376 |
| GPR26-Tango | 0.904 | 0.904 | 1.325 | 1.296 | 0.734 | 1.262 | 1.117 | 1.358 |
| GPR27-Tango | 0.133 | 0.133 | −1.369 | −0.147 | 0.000 | −0.147 | −0.784 | 0.133 |
| GPR3-Tango | −0.295 | −0.034 | 0.013 | −0.113 | 0.507 | −0.222 | 0.180 | 0.138 |
| GPR31-Tango | −1.427 | −0.177 | −0.804 | −0.487 | −0.082 | −0.370 | −0.007 | 0.158 |
| GPR32-Tango | 0.055 | 0.752 | 1.123 | 0.922 | 0.472 | 0.625 | 1.223 | 1.090 |
| GPR34-Tango | 0.659 | 1.096 | 0.904 | 0.981 | 0.943 | 0.808 | 0.924 | 1.067 |
| GPR35-Tango | −0.070 | 0.355 | 0.603 | 0.463 | 0.355 | 0.501 | 0.287 | 0.253 |
| GPR37-Tango | 0.087 | 0.369 | 0.323 | 0.272 | 0.421 | 0.200 | 0.219 | 0.355 |
| GPR37L1-Tango | 0.146 | 0.357 | 0.518 | 0.451 | 0.144 | 0.573 | 0.233 | 0.290 |
| GPR39-Tango | −0.678 | 0.672 | −0.167 | 0.820 | −0.415 | 0.150 | −0.678 | 0.209 |
| GPR4-Tango | −0.165 | −0.215 | 0.265 | −0.061 | −0.162 | −0.182 | −0.170 | 0.083 |
| GPR44-Tango | 0.439 | 0.544 | 0.205 | 1.140 | 0.609 | −0.795 | 0.439 | 0.072 |
| GPR45-Tango | −0.166 | 0.538 | 0.149 | 1.112 | −0.431 | −0.325 | 0.221 | 0.265 |
| GPR50-Tango | 0.620 | 0.678 | 1.181 | 1.150 | 0.754 | 1.000 | 0.720 | 0.954 |
| GPR52-Tango | −0.497 | 0.585 | 0.338 | −0.082 | 0.188 | 0.134 | −0.585 | 0.415 |
| GPR55-Tango | −0.349 | 0.090 | −0.196 | −0.247 | 0.553 | −0.109 | −0.143 | −0.156 |
| GPR56-Tango | −0.265 | −0.116 | 0.166 | 0.044 | 0.114 | 0.099 | 0.306 | 0.314 |
| GPR6-Tango | 0.338 | 0.735 | 0.748 | 0.560 | 0.802 | 0.097 | 0.609 | 0.772 |
| GPR61-Tango | −0.566 | −0.418 | −0.243 | −0.199 | −0.512 | −0.743 | −0.692 | −0.426 |
| GPR62-Tango | −0.152 | 0.766 | 1.000 | 0.138 | 0.000 | 1.202 | −0.152 | 1.632 |
| GPR63-Tango | −0.143 | −0.099 | −0.078 | −0.047 | −0.165 | 0.086 | −0.089 | −0.054 |
| GPR64-Tango | −0.132 | −0.129 | −0.145 | 0.046 | −0.468 | −0.111 | −0.092 | −0.046 |
| GPR65-Tango | −0.266 | −0.163 | −0.199 | −0.163 | −0.366 | −0.433 | −0.399 | −0.409 |
| GPR68-Tango | −0.532 | −0.261 | −0.318 | −0.381 | −0.825 | −0.453 | −0.372 | −0.310 |
| GPR75- Tango | −0.385 | −0.546 | −0.149 | −0.125 | −0.497 | −0.065 | −0.103 | −0.106 |
| GPR77-Tango | −0.494 | −0.549 | −0.108 | −0.525 | 0.284 | −0.280 | −0.030 | −0.178 |
| GPR78-Tango | −0.609 | 0.018 | −0.137 | −0.006 | −0.257 | 0.165 | −0.111 | 0.187 |
| GPR82-Tango | 0.246 | 0.415 | 0.291 | 0.143 | −0.139 | −0.043 | 0.140 | 0.307 |
| GPR83-Tango | −0.520 | 0.028 | −0.190 | −0.299 | −0.557 | −0.222 | −0.087 | −0.133 |
| GPR84-Tango | 0.032 | 0.149 | 0.052 | 0.158 | 0.128 | −0.108 | 0.296 | 0.201 |
| GPR85-Tango | −0.163 | 0.034 | 0.405 | 0.211 | 0.826 | 0.582 | −0.056 | 0.311 |
| GPR87-Tango | 0.042 | 0.392 | 0.089 | 0.017 | 0.331 | 0.231 | 0.031 | 0.097 |
| GPR88-Tango | −0.444 | −0.598 | −0.993 | −0.726 | −0.240 | −0.661 | −0.794 | −0.661 |
| GPR97-Tango | 0.106 | 0.334 | 0.061 | 0.626 | 0.705 | 0.734 | 0.476 | 1.149 |
| GPRC5A-Tango | −0.657 | −0.308 | −0.243 | −0.414 | −0.803 | −0.492 | −0.555 | −0.421 |
| GPRC5B-Tango | −0.616 | −0.293 | −0.348 | −0.417 | −0.699 | −0.525 | −0.411 | −0.367 |
| GPRC5C-Tango | −0.222 | −0.138 | −0.142 | −0.106 | −0.195 | 0.035 | −0.339 | −0.336 |
| GPRC5D-Tango | −0.477 | −0.125 | 0.053 | −0.007 | 0.019 | −0.148 | 0.032 | −0.110 |
| GPRC6A-Tango | −0.494 | −0.265 | −0.290 | −0.434 | −0.834 | −0.621 | −0.519 | −0.462 |
| GRM7-Tango | −0.399 | −0.396 | −0.427 | −0.425 | −0.622 | −0.395 | −0.422 | −0.469 |
| GRM8-Tango | −0.077 | −0.045 | −0.026 | −0.125 | −0.746 | −0.478 | 0.019 | 0.095 |
| GRPR-Tango | −0.383 | −0.383 | −0.089 | 0.309 | −0.319 | 0.349 | 0.214 | 0.030 |

| PRESTO-Tango | Peptoniphilus | Bacteroids | Bacteroids | Bacteroids | Bacteroides | Bacteroides | Bacteroides | Bacteroides |
|---|---|---|---|---|---|---|---|---|
| with Gifu Medium | spp-3# | fragilis-1# | fragilis-2# | fragilis-3# | ovatus-1# | ovatus-2# | ovatus-3# | ovatus-4# |
| GPR1-Tango | −0.106 | −0.726 | 0.188 | −0.871 | −0.054 | −0.154 | −0.633 | −0.149 |
| GPR101-Tango | 0.070 | 0.372 | 0.282 | 0.381 | 0.656 | 0.501 | 0.579 | 0.429 |
| GPR110-Tango | 0.167 | 0.402 | −0.121 | 0.162 | 0.429 | 0.379 | 0.312 | 0.229 |
| GPR111-Tango | 1.357 | 0.080 | 0.181 | −2.392 | 0.571 | 0.373 | 1.181 | 1.682 |
| GPR113-Tango | 0.393 | 0.377 | −0.562 | −1.438 | 0.580 | 0.346 | 0.620 | 0.794 |
| GPR114-Tango | 0.165 | 0.165 | −0.700 | −0.420 | 0.325 | 0.580 | 0.796 | 0.446 |
| GPR115-Tango | −0.309 | −0.173 | −0.289 | −2.399 | 0.077 | 0.282 | 0.145 | −0.208 |
| GPR116-Tango | 0.224 | 0.249 | −0.024 | −2.192 | 0.414 | 0.661 | 0.479 | 0.347 |
| GPR119-Tango | 0.714 | 0.941 | −0.218 | −0.434 | 1.251 | 0.816 | 0.986 | 1.379 |
| GPR12-Tango | 0.224 | 0.042 | 0.327 | −0.099 | 0.440 | −0.068 | 0.401 | 0.212 |
| GPR120-Tango | 0.491 | 0.314 | 0.745 | 1.112 | 1.112 | 0.491 | 0.878 | 0.598 |
| GPR123-Tango | 0.099 | 0.776 | −0.399 | −0.033 | 1.150 | 0.601 | 0.786 | 0.510 |
| GPR124-Tango | 0.611 | 0.805 | 0.850 | 1.143 | 0.850 | 1.165 | 0.642 | 0.750 |
| GPR125-Tango | −0.364 | −0.224 | −0.346 | −2.458 | 0.060 | 0.297 | 0.080 | −0.149 |
| GPR126-Tango | 0.071 | 0.483 | 0.245 | 1.061 | 0.630 | 1.227 | 0.570 | 0.668 |
| GPR132-Tango | −0.514 | 0.383 | −1.123 | −2.801 | 0.151 | 0.192 | 0.313 | −0.259 |
| GPR133-Tango | −0.268 | −0.081 | −0.345 | −1.810 | 0.059 | 0.193 | 0.044 | −0.127 |
| GPR135-Tango | −0.257 | 0.011 | −0.745 | −0.585 | 0.032 | −1.011 | −0.300 | −0.257 |
| GPR141-Tango | −0.636 | −0.150 | 0.725 | 0.364 | 0.308 | 0.776 | 0.522 | 0.397 |
| GPR142-Tango | 1.064 | 0.563 | 0.415 | 0.835 | 0.713 | 0.563 | 0.760 | 1.136 |
| GPR143-Tango | 0.149 | 0.492 | −1.030 | −2.396 | 0.526 | 0.173 | 0.660 | 0.325 |
| GPR144-Tango | 0.323 | −0.617 | −0.319 | −2.303 | −0.272 | −0.367 | 0.486 | 0.513 |
| GPR146-Tango | −0.234 | 0.766 | 0.202 | −2.737 | 0.379 | −0.515 | 1.263 | 0.766 |
| GPR148-Tango | 0.528 | −0.339 | 0.783 | −0.967 | −0.104 | 0.097 | −0.967 | 0.274 |
| GPR149-Tango | 1.331 | 1.078 | 0.953 | −0.568 | 1.213 | 1.294 | 0.791 | 1.674 |
| GPR15-Tango | −0.146 | 0.057 | −1.133 | −1.474 | 0.392 | −0.146 | 0.347 | −0.506 |
| GPR150-Tango | 0.621 | 0.823 | 0.387 | 1.000 | 0.823 | 1.254 | 1.000 | 1.387 |
| GPR151-Tango | 0.616 | 0.924 | 0.260 | 0.410 | 1.052 | 0.989 | 0.722 | 0.657 |
| GPR152-Tango | 0.477 | 0.521 | −0.498 | −2.031 | 0.317 | −0.049 | 0.424 | 0.555 |
| GPR153-Tango | −0.229 | 0.115 | −0.027 | −2.151 | 0.185 | 0.204 | 0.220 | −0.057 |
| GPR156-Tango | 0.298 | 0.877 | 0.815 | 1.121 | 0.771 | 0.834 | 0.616 | 0.790 |
| GPR157-Tango | 0.739 | 0.953 | 0.008 | −1.846 | 0.710 | 0.710 | 1.082 | 1.126 |
| GPR158-Tango | −0.010 | 0.741 | −0.435 | −0.224 | 0.425 | 0.787 | 0.294 | 0.220 |
| GPR160-Tango | −0.157 | 0.141 | −0.810 | −0.373 | 0.241 | 0.275 | 0.226 | 0.034 |
| GPR161-Tango | −0.719 | −0.278 | −0.459 | −0.434 | −0.013 | 0.313 | 0.074 | −0.243 |
| GPR162-Tango | −0.403 | 0.193 | −0.807 | 0.128 | 0.819 | −0.062 | 0.193 | 0.690 |
| GPR17-Tango | −0.053 | 0.092 | −0.024 | −2.296 | 0.271 | 0.245 | 0.108 | −0.006 |
| GPR171-Tango | 0.543 | 1.192 | −1.046 | −2.319 | 1.018 | 0.677 | 1.438 | 0.817 |
| GPR173-Tango | 0.274 | 0.896 | −0.104 | −0.619 | 1.066 | 0.783 | 1.000 | 1.246 |
| GPR174-Tango | 0.802 | 0.535 | −0.263 | −0.963 | 1.483 | 0.300 | 1.214 | 1.503 |
| GPR18-Tango | 1.114 | 1.184 | −0.314 | −1.849 | 1.399 | 0.816 | 1.433 | 1.027 |
| GPR182-Tango | −0.184 | 0.289 | 0.806 | 0.130 | 0.524 | 0.390 | 0.474 | −0.067 |
| GPR183-Tango | 0.076 | 0.199 | −1.177 | −0.391 | 0.444 | 0.246 | 0.356 | 0.497 |
| GPR19-Tango | −0.406 | 0.369 | −0.993 | −0.441 | 0.737 | 0.324 | 0.771 | 0.254 |
| GPR20-Tango | 0.267 | −0.263 | −1.054 | −4.170 | 0.395 | 0.000 | 0.267 | 0.267 |
| GPR21-Tango | 1.350 | 1.263 | −0.234 | −2.737 | 1.632 | 0.888 | 2.036 | 1.433 |
| GPR22-Tango | 0.002 | 0.735 | 0.107 | 0.486 | 0.661 | 0.844 | 0.747 | 0.474 |
| GPR25-Tango | 0.745 | 1.000 | 0.491 | −1.040 | 0.491 | 0.878 | 0.598 | −0.122 |
| GPR26-Tango | 0.904 | 0.791 | 0.292 | −1.354 | 0.765 | 0.891 | 0.928 | 1.482 |
| GPR27-Tango | 1.000 | −0.147 | 0.569 | −1.369 | −0.495 | −0.784 | −0.495 | 0.368 |
| GPR3-Tango | −0.277 | 0.130 | 1.042 | 0.197 | 0.711 | 0.091 | 0.762 | 0.423 |
| GPR31-Tango | −0.333 | −0.007 | −0.370 | −0.129 | 0.184 | 0.649 | 0.451 | −0.227 |
| GPR32-Tango | 1.214 | 0.223 | −0.078 | −1.939 | 0.651 | 0.537 | 1.657 | 1.295 |
| GPR34-Tango | 1.130 | 0.751 | 0.191 | −1.655 | 0.850 | 0.683 | 1.365 | 1.130 |
| GPR35-Tango | 0.280 | 0.429 | −0.105 | −2.209 | 0.479 | 0.147 | 0.644 | 0.533 |
| GPR37-Tango | 0.262 | 0.269 | 0.422 | −1.728 | 0.456 | 0.669 | 0.764 | 0.288 |
| GPR37L1-Tango | 0.416 | 0.321 | −0.185 | −1.049 | 0.705 | 0.831 | 0.534 | 0.431 |
| GPR39-Tango | −0.245 | 0.858 | −0.415 | −0.508 | 0.714 | 0.087 | 1.000 | 1.180 |
| GPR4-Tango | −0.138 | 0.164 | −0.072 | −0.045 | 0.551 | 0.577 | 0.458 | 0.129 |
| GPR44-Tango | 0.872 | 0.072 | 0.609 | −0.359 | 1.072 | 0.439 | 0.872 | −0.075 |
| GPR45-Tango | 0.331 | 0.265 | −0.362 | −2.528 | 0.006 | −0.431 | 0.538 | 1.009 |
| GPR50-Tango | 0.657 | 0.966 | −0.409 | −1.907 | 0.900 | 0.415 | 1.564 | 1.050 |
| GPR52-Tango | 0.415 | 0.852 | 1.377 | 0.338 | 1.115 | −0.337 | 0.585 | 1.010 |
| GPR55-Tango | −0.227 | 0.550 | −0.401 | −1.858 | 0.684 | 0.740 | 0.628 | 0.021 |
| GPR56-Tango | 0.224 | 0.463 | 0.174 | 0.421 | 0.719 | 0.572 | 0.699 | 0.519 |
| GPR6-Tango | 0.144 | 1.060 | 1.051 | 0.103 | 1.275 | 1.070 | 0.918 | 0.434 |
| GPR61-Tango | −0.313 | −0.116 | −0.351 | −0.484 | 0.248 | 0.055 | 0.074 | 0.049 |
| GPR62-Tango | −1.737 | 0.138 | 0.138 | −0.737 | 0.138 | 0.485 | 0.766 | 0.485 |
| GPR63-Tango | −0.187 | −0.138 | −0.112 | −2.561 | 0.145 | 0.379 | 0.225 | 0.099 |
| GPR64-Tango | −0.093 | −0.453 | −0.356 | −2.602 | −0.074 | 0.259 | −0.184 | −0.060 |
| GPR65-Tango | −0.388 | −0.002 | −0.553 | −2.106 | 0.088 | 0.354 | 0.065 | −0.277 |
| GPR68-Tango | −0.244 | 0.107 | −1.270 | −2.578 | 0.128 | 0.473 | 0.231 | −0.296 |
| GPR75- Tango | 0.036 | −0.149 | −0.722 | −0.506 | −0.069 | −0.335 | −0.097 | −0.118 |
| GPR77-Tango | −0.300 | 0.022 | 0.144 | −0.178 | 0.259 | 0.444 | 0.360 | 0.135 |
| GPR78-Tango | −0.177 | 0.689 | −0.772 | 0.525 | 0.745 | 0.427 | 0.312 | 0.385 |
| GPR82-Tango | 0.087 | 0.316 | 0.489 | 0.070 | 0.503 | 0.415 | 0.413 | 0.018 |
| GPR83-Tango | −0.123 | 0.132 | −0.687 | −2.626 | 0.250 | 0.075 | 0.429 | −0.020 |
| GPR84-Tango | −0.035 | 0.085 | 0.189 | −2.181 | 0.398 | 0.400 | 0.400 | −0.033 |
| GPR85-Tango | 0.356 | 0.188 | −0.008 | −1.807 | 0.470 | 0.775 | 0.817 | 0.593 |
| GPR87-Tango | 0.004 | 0.237 | −0.257 | −0.925 | 0.153 | 0.588 | 0.289 | 0.122 |
| GPR88-Tango | −0.119 | 0.183 | −0.444 | −0.661 | 0.432 | −0.240 | 0.451 | −0.240 |
| GPR97-Tango | 0.031 | 0.061 | 0.676 | 0.860 | 1.225 | 0.626 | 0.911 | 0.834 |
| GPRC5A-Tango | −0.344 | 0.316 | −0.509 | −0.541 | 0.424 | 0.326 | 0.436 | 0.042 |
| GPRC5B-Tango | −0.562 | 0.327 | −0.528 | −0.626 | 0.345 | 0.309 | 0.358 | −0.092 |
| GPRC5C-Tango | −0.073 | 0.375 | −0.612 | −2.665 | 0.386 | 0.351 | 0.575 | 0.147 |
| GPRC5D-Tango | 0.053 | 0.108 | −0.062 | −2.334 | 0.570 | 0.399 | 0.723 | 0.249 |
| GPRC6A-Tango | −0.395 | −0.119 | −0.702 | −2.958 | −0.220 | −0.107 | −0.008 | −0.370 |
| GRM7-Tango | −0.495 | −0.381 | −0.783 | −2.520 | −0.142 | 0.130 | −0.161 | −0.369 |
| GRM8-Tango | 0.103 | 0.226 | −0.824 | −2.174 | −0.051 | 0.440 | 0.207 | −0.109 |
| GRPR-Tango | 0.175 | 0.062 | −0.870 | −3.356 | 0.183 | −0.179 | 0.013 | 0.206 |

| PRESTO-Tango | B. | B. | B. | B. | B. | B. | Bacteroides | Bacteroides |
|---|---|---|---|---|---|---|---|---|
| with Gifu Medium | ovatus-5# | thetao-1# | thetao-2# | eggerthii | Caccae | copri | uniformis-1# | uniformis-2# |
| GPR1-Tango | 0.512 | −0.284 | −0.718 | −1.254 | −0.564 | −0.682 | −0.981 | −0.335 |
| GPR101-Tango | 0.565 | 0.251 | 0.224 | 0.262 | 0.130 | −0.375 | 0.649 | 0.081 |
| GPR110-Tango | 0.739 | 0.083 | 0.271 | 0.267 | 0.415 | −0.330 | 0.837 | 0.189 |
| GPR111-Tango | 1.112 | 0.373 | 0.373 | 0.515 | 0.958 | 2.305 | 0.466 | 0.833 |
| GPR113-Tango | 0.104 | 0.870 | −0.258 | 0.726 | −0.007 | −0.921 | 0.418 | 0.207 |
| GPR114-Tango | 0.642 | 0.399 | 0.580 | 0.325 | 0.077 | 0.952 | 0.446 | 0.515 |
| GPR115-Tango | 0.016 | −0.095 | −0.161 | −0.052 | −0.135 | −0.424 | 0.126 | −0.165 |
| GPR116-Tango | 0.253 | 0.283 | 0.310 | 0.367 | 0.166 | 0.932 | 0.303 | 0.231 |
| GPR119-Tango | 0.864 | 1.029 | 1.176 | 0.714 | 0.660 | −0.837 | 0.766 | 1.029 |
| GPR12-Tango | 0.564 | −0.068 | −0.436 | 0.280 | 0.212 | 0.014 | 0.000 | −0.039 |
| GPR120-Tango | 0.314 | 1.000 | 1.217 | 0.878 | 0.112 | 1.697 | −0.122 | 0.598 |
| GPR123-Tango | 1.279 | 0.403 | 0.158 | 0.796 | 0.546 | 0.214 | 0.698 | −0.069 |
| GPR124-Tango | 0.952 | 0.805 | 0.165 | 0.805 | 0.077 | −0.485 | 0.832 | 0.515 |
| GPR125-Tango | −0.187 | −0.205 | −0.119 | −0.077 | −0.290 | −0.263 | 0.035 | −0.307 |
| GPR126-Tango | 1.516 | 0.285 | −0.115 | −0.103 | −0.187 | 0.588 | 0.891 | −0.019 |
| GPR132-Tango | 0.326 | 0.401 | 0.399 | 0.479 | 0.013 | −1.115 | 0.196 | −0.068 |
| GPR133-Tango | −0.044 | −0.025 | −0.105 | −0.192 | −0.211 | −0.331 | −0.006 | −0.146 |
| GPR135-Tango | −0.411 | −0.441 | −0.411 | −0.140 | −0.521 | −1.086 | −0.327 | −0.411 |
| GPR141-Tango | 0.956 | 0.237 | 0.014 | 0.097 | −0.301 | 0.522 | 0.541 | −0.199 |
| GPR142-Tango | 0.355 | 0.713 | 0.293 | 0.160 | 0.112 | −0.303 | 0.791 | 0.878 |
| GPR143-Tango | 0.547 | 0.580 | 0.192 | 0.539 | 0.370 | −0.436 | 0.455 | 0.530 |
| GPR144-Tango | −0.523 | −0.112 | −0.367 | 0.173 | 0.373 | −0.560 | −0.319 | 0.104 |
| GPR146-Tango | 0.202 | −1.152 | 0.766 | 1.104 | 0.379 | −0.862 | −0.234 | 0.485 |
| GPR148-Tango | 0.097 | 0.381 | 1.066 | −0.339 | −1.256 | 0.097 | 1.066 | 1.066 |
| GPR149-Tango | 0.652 | −0.047 | 0.280 | 0.523 | 0.570 | 0.045 | 0.849 | 1.266 |
| GPR15-Tango | 0.234 | −0.336 | −0.662 | −0.146 | −0.411 | −0.336 | −0.146 | −0.458 |
| GPR150-Tango | 0.823 | 0.107 | −2.115 | 1.107 | 1.000 | −0.241 | 1.387 | 1.254 |
| GPR151-Tango | 0.531 | 0.574 | −0.011 | 1.052 | 0.487 | −0.076 | 0.989 | 0.361 |
| GPR152-Tango | 0.441 | 0.477 | 0.430 | 0.607 | 0.497 | −0.195 | 0.327 | 0.516 |
| GPR153-Tango | 0.255 | 0.157 | 0.200 | 0.194 | −0.146 | −0.432 | 0.191 | −0.072 |
| GPR156-Tango | 1.322 | 0.646 | 0.185 | 0.424 | 0.490 | 1.097 | 0.858 | 0.831 |
| GPR157-Tango | 0.811 | 0.453 | 0.837 | 1.643 | 0.508 | −0.459 | 1.167 | 0.633 |
| GPR158-Tango | 0.770 | 0.656 | 0.294 | 0.068 | −0.395 | −0.517 | 1.068 | 0.687 |
| GPR160-Tango | 0.436 | 0.047 | 0.016 | 0.015 | −0.139 | −0.705 | 0.199 | −0.098 |
| GPR161-Tango | 0.412 | −0.170 | −0.025 | −0.171 | −0.197 | −0.435 | 0.124 | −0.391 |
| GPR162-Tango | 0.372 | 0.860 | 0.409 | 0.900 | 0.315 | −0.941 | 0.012 | −0.469 |
| GPR17-Tango | 0.276 | −0.097 | −0.181 | 0.010 | −0.051 | −0.141 | −0.049 | 0.033 |
| GPR171-Tango | 0.631 | 0.576 | 0.685 | 1.162 | 0.600 | −0.404 | 0.929 | 0.564 |
| GPR173-Tango | 0.528 | 0.896 | 0.661 | 0.896 | 0.896 | −0.967 | 0.274 | 0.381 |
| GPR174-Tango | 0.509 | 0.509 | 0.429 | 0.509 | 1.054 | −0.453 | 0.834 | 0.429 |
| GPR18-Tango | 0.975 | 0.867 | 0.499 | 1.190 | 1.325 | −0.104 | 0.975 | 0.949 |
| GPR182-Tango | 0.197 | 0.323 | 0.085 | 0.153 | −0.116 | −0.401 | 0.480 | 0.089 |
| GPR183-Tango | 0.340 | 0.217 | 0.388 | 0.773 | 0.577 | −0.418 | 0.199 | −0.038 |
| GPR19-Tango | 0.539 | 0.163 | 0.260 | 0.216 | −0.118 | −0.923 | 0.598 | 0.195 |
| GPR20-Tango | 0.800 | −0.329 | −1.433 | −1.231 | 0.152 | −1.948 | 0.152 | 0.333 |
| GPR21-Tango | 1.104 | 1.000 | 1.170 | 1.585 | 1.104 | −0.515 | 1.433 | 1.000 |
| GPR22-Tango | 1.129 | 0.692 | −0.081 | 0.136 | 0.180 | −0.346 | 0.786 | 0.296 |
| GPR25-Tango | 1.000 | 1.463 | 0.112 | 0.598 | −0.122 | −2.624 | 1.463 | 1.813 |
| GPR26-Tango | 0.904 | 0.450 | 0.461 | 0.509 | 0.683 | 0.658 | 0.605 | 0.710 |
| GPR27-Tango | −0.495 | −0.147 | 0.133 | 0.133 | 0.368 | 0.500 | 0.368 | −0.495 |
| GPR3-Tango | 0.507 | 0.344 | 0.295 | 0.284 | 0.033 | −0.978 | 0.304 | 0.300 |
| GPR31-Tango | 1.036 | 0.118 | −0.730 | −0.007 | −0.279 | 0.563 | 0.621 | 0.036 |
| GPR32-Tango | 1.564 | 0.828 | 0.625 | 0.939 | 0.932 | 0.720 | 1.191 | 1.445 |
| GPR34-Tango | 1.049 | 0.421 | 1.113 | 1.239 | 1.084 | 0.545 | 1.067 | 0.659 |
| GPR35-Tango | 0.784 | 0.269 | 0.472 | 0.569 | 0.398 | −0.889 | 0.475 | 0.648 |
| GPR37-Tango | 0.565 | 0.252 | 0.191 | 0.398 | 0.183 | −0.394 | 0.487 | 0.559 |
| GPR37L1-Tango | 0.367 | 0.091 | 0.063 | 0.399 | 0.374 | −0.020 | 0.321 | 0.484 |
| GPR39-Tango | 0.672 | 0.555 | 0.629 | 0.409 | 0.714 | 0.150 | 0.672 | 0.629 |
| GPR4-Tango | 0.491 | 0.221 | 0.048 | 0.164 | 0.062 | −0.601 | 0.440 | 0.414 |
| GPR44-Tango | 0.950 | 0.790 | 0.072 | 0.327 | −0.075 | 0.544 | 1.072 | 0.439 |
| GPR45-Tango | 0.750 | −0.303 | −0.431 | −0.078 | −0.041 | −0.641 | 0.250 | 0.101 |
| GPR50-Tango | 0.882 | 0.936 | 0.291 | 0.983 | 0.864 | −0.440 | 0.794 | 1.077 |
| GPR52-Tango | 1.115 | 0.239 | −0.585 | −0.082 | −0.020 | 0.980 | 0.040 | 0.239 |
| GPR55-Tango | 0.676 | 0.392 | 0.323 | 0.640 | 0.241 | 0.267 | 0.342 | 0.005 |
| GPR56-Tango | 1.220 | 0.646 | 0.278 | −0.066 | 0.349 | 0.725 | 1.103 | 0.327 |
| GPR6-Tango | 0.752 | 0.329 | −0.115 | 0.575 | 0.306 | −0.463 | 0.434 | 0.602 |
| GPR61-Tango | 0.183 | −0.050 | −0.343 | −0.006 | −0.172 | −0.743 | 0.097 | −0.537 |
| GPR62-Tango | 1.202 | 1.202 | 1.000 | 1.202 | −0.737 | −0.737 | −0.152 | −0.737 |
| GPR63-Tango | 0.138 | −0.052 | −0.052 | 0.066 | −0.429 | −0.171 | −0.035 | −0.077 |
| GPR64-Tango | −0.290 | −0.440 | −0.252 | −0.108 | −0.350 | −0.009 | −0.132 | −0.189 |
| GPR65-Tango | −0.015 | 0.095 | −0.074 | 0.104 | −0.133 | −0.613 | 0.042 | −0.229 |
| GPR68-Tango | −0.070 | 0.034 | 0.183 | 0.224 | 0.034 | −1.656 | 0.148 | 0.174 |
| GPR75- Tango | 0.142 | −0.219 | −0.416 | −0.072 | 0.057 | −0.215 | −0.346 | 0.181 |
| GPR77-Tango | 0.571 | 0.151 | −0.274 | −0.018 | −0.139 | −0.393 | −0.004 | 0.343 |
| GPR78-Tango | 0.757 | 0.347 | 0.018 | −0.039 | −0.431 | −0.609 | 0.486 | 0.245 |
| GPR82-Tango | 0.379 | 0.042 | −0.081 | 0.450 | 0.093 | −0.174 | 0.513 | −0.224 |
| GPR83-Tango | 0.322 | 0.304 | 0.267 | 0.488 | 0.241 | −0.199 | 0.362 | 0.205 |
| GPR84-Tango | 0.199 | 0.063 | 0.043 | 0.057 | −0.102 | 0.166 | 0.067 | 0.127 |
| GPR85-Tango | 0.866 | 0.620 | 0.522 | 0.908 | 0.843 | 0.369 | 0.600 | 0.712 |
| GPR87-Tango | 0.324 | 0.137 | −0.110 | 0.075 | 0.080 | −0.781 | 0.146 | 0.294 |
| GPR88-Tango | 0.402 | 0.007 | −0.119 | −0.339 | −0.240 | 0.451 | −0.391 | −0.539 |
| GPR97-Tango | 1.436 | 0.499 | 0.476 | −0.016 | 0.297 | 0.656 | 1.091 | 0.564 |
| GPRC5A-Tango | 0.917 | 0.163 | −0.327 | 0.172 | −0.274 | 0.070 | 0.591 | −0.135 |
| GPRC5B-Tango | 0.711 | 0.107 | −0.152 | −0.108 | −0.064 | −0.758 | 0.382 | −0.215 |
| GPRC5C-Tango | 0.523 | 0.359 | 0.187 | 0.476 | 0.271 | −0.342 | 0.299 | 0.062 |
| GPRC5D-Tango | 0.495 | 0.095 | 0.078 | 0.091 | 0.078 | −0.262 | 0.419 | 0.014 |
| GPRC6A-Tango | 0.068 | −0.087 | −0.002 | −0.020 | −0.324 | −0.413 | 0.038 | −0.249 |
| GRM7-Tango | −0.105 | −0.057 | −0.122 | −0.093 | −0.326 | −0.735 | −0.463 | −0.225 |
| GRM8-Tango | 0.081 | 0.075 | 0.184 | 0.149 | 0.218 | 1.132 | 0.292 | 0.365 |
| GRPR-Tango | 0.170 | −0.004 | −0.544 | −0.394 | 0.183 | −0.519 | −0.190 | 0.633 |

| PRESTO-Tango | Bacteroides | Bacteroides | Bacteroides | Bacteroides | Parabacteroides | Parabacteroides | Fusobacterium | Fusobacterium |
|---|---|---|---|---|---|---|---|---|
| with Gifu Medium | uniformis-3# | UC-1# | UC-2# | UC-3# | distasonis-1# | distasonis-2# | spp-1# | spp-2# |
| GPR1-Tango | −0.906 | 0.160 | −0.154 | 0.128 | −0.073 | −0.645 | −0.817 | −0.344 |
| GPR101-Tango | 0.302 | 0.870 | 0.231 | 0.191 | 0.295 | 0.164 | −0.587 | 0.168 |
| GPR110-Tango | 0.316 | 0.495 | 0.283 | 0.489 | 0.267 | −0.025 | −0.870 | −0.100 |
| GPR111-Tango | 0.958 | 1.594 | 0.915 | 1.162 | 0.786 | 0.405 | −0.505 | 1.181 |
| GPR113-Tango | 0.393 | 0.681 | 0.558 | 0.594 | 0.668 | −0.242 | −1.332 | 0.478 |
| GPR114-Tango | 0.165 | 0.515 | 0.446 | 0.702 | 0.796 | 0.580 | −2.338 | −0.082 |
| GPR115-Tango | −0.090 | 0.142 | −0.027 | 0.088 | −0.141 | −0.265 | −0.958 | −0.304 |
| GPR116-Tango | 0.388 | 0.482 | 0.322 | 0.446 | 0.222 | 0.253 | −0.359 | 0.077 |
| GPR119-Tango | 0.766 | 0.444 | 0.941 | 0.864 | 0.816 | 0.766 | 1.614 | 1.251 |
| GPR12-Tango | −0.188 | −0.140 | 0.368 | 0.338 | 0.273 | 0.106 | −1.529 | −0.455 |
| GPR120-Tango | −0.402 | 1.000 | 0.112 | 1.623 | 0.314 | 0.598 | −0.402 | −0.402 |
| GPR123-Tango | 0.142 | 0.716 | 1.017 | 0.489 | 0.390 | 0.312 | −1.588 | 0.126 |
| GPR124-Tango | 0.000 | 0.547 | 0.458 | 0.547 | 0.287 | 0.287 | 1.469 | 0.287 |
| GPR125-Tango | −0.163 | 0.078 | −0.190 | −0.085 | −0.077 | −0.300 | −0.311 | −0.071 |
| GPR126-Tango | 0.359 | 0.346 | 0.384 | 0.071 | 0.236 | −0.295 | 0.040 | 0.165 |
| GPR132-Tango | −0.188 | −0.184 | 0.157 | 0.178 | −0.349 | −0.145 | −1.243 | −0.607 |
| GPR133-Tango | −0.112 | 0.185 | −0.081 | 0.027 | −0.029 | −0.336 | −0.724 | −0.230 |
| GPR135-Tango | −0.926 | −0.156 | −0.026 | −0.521 | −0.490 | −0.521 | −1.807 | −1.132 |
| GPR141-Tango | −0.825 | 0.200 | 0.056 | 0.308 | −0.825 | −0.104 | −0.825 | −0.508 |
| GPR142-Tango | 0.760 | 0.878 | 0.791 | 0.713 | 0.760 | 0.112 | −0.303 | 0.509 |
| GPR143-Tango | 0.029 | 0.322 | 0.375 | 0.486 | −0.008 | 0.135 | −1.874 | −0.225 |
| GPR144-Tango | 0.373 | 0.196 | 0.323 | 0.292 | −0.242 | −0.417 | 1.743 | 0.557 |
| GPR146-Tango | 0.000 | 0.632 | 0.202 | −1.152 | 0.202 | −1.152 | −1.737 | 0.766 |
| GPR148-Tango | 0.783 | 0.783 | 0.661 | 0.274 | −0.104 | 0.097 | 1.000 | 0.661 |
| GPR149-Tango | 1.266 | 1.034 | 1.280 | 0.811 | 1.227 | 0.868 | 1.126 | 1.884 |
| GPR15-Tango | −0.171 | −0.293 | 0.034 | −0.211 | −0.381 | −0.556 | −2.265 | −0.699 |
| GPR150-Tango | 0.621 | 0.621 | −0.241 | 0.823 | 0.621 | −1.115 | 0.387 | 0.387 |
| GPR151-Tango | 0.052 | 0.797 | 0.574 | 0.574 | −0.343 | 0.260 | −0.672 | 0.052 |
| GPR152-Tango | 0.205 | −0.049 | 0.602 | 0.714 | 0.404 | 0.629 | −1.233 | −0.139 |
| GPR153-Tango | 0.079 | 0.132 | 0.138 | 0.141 | 0.021 | −0.085 | −1.651 | −0.161 |
| GPR156-Tango | 0.666 | 0.931 | 0.801 | 0.764 | 0.662 | 0.023 | 0.670 | 0.600 |
| GPR157-Tango | 0.937 | 0.311 | 1.167 | 1.126 | 0.862 | 0.888 | 0.126 | 0.653 |
| GPR158-Tango | 0.504 | 0.504 | 0.245 | 0.403 | 0.123 | −0.259 | 0.096 | 0.245 |
| GPR160-Tango | −0.326 | 0.357 | 0.025 | −0.089 | −0.102 | −0.462 | −1.161 | −0.366 |
| GPR161-Tango | −0.357 | 0.075 | −0.261 | −0.260 | −0.626 | −0.601 | −1.329 | −0.708 |
| GPR162-Tango | 0.315 | 0.613 | 0.255 | 0.255 | −0.469 | −0.140 | −2.435 | 0.059 |
| GPR17-Tango | −0.004 | 0.378 | 0.152 | 0.215 | 0.133 | −0.151 | −1.250 | 0.114 |
| GPR171-Tango | 0.432 | 1.042 | 0.769 | 0.769 | 0.913 | 0.354 | −1.882 | 0.838 |
| GPR173-Tango | 1.407 | 1.000 | 1.159 | −0.104 | 0.896 | 0.783 | −2.841 | 0.097 |
| GPR174-Tango | 0.895 | 0.429 | 0.645 | 1.198 | 1.072 | 1.098 | −0.963 | 0.387 |
| GPR18-Tango | 1.084 | 1.071 | 0.543 | 1.120 | 1.014 | 0.499 | −0.808 | 1.229 |
| GPR182-Tango | 0.021 | 0.639 | 0.065 | 0.304 | 0.182 | −0.238 | −0.600 | −0.042 |
| GPR183-Tango | −0.446 | 0.356 | 0.127 | 0.246 | 0.095 | 0.199 | −1.374 | −0.418 |
| GPR19-Tango | 0.238 | 0.323 | 0.305 | 0.535 | −0.103 | −0.332 | −1.182 | −0.396 |
| GPR20-Tango | −0.667 | −0.329 | 0.333 | −0.170 | −0.170 | 0.000 | −1.667 | −0.433 |
| GPR21-Tango | 1.511 | 1.000 | 0.485 | 1.433 | 0.379 | 0.888 | −1.152 | 1.888 |
| GPR22-Tango | 0.255 | 0.569 | 0.576 | 0.398 | 0.180 | −0.234 | 0.142 | −0.019 |
| GPR25-Tango | 1.112 | 0.598 | 1.217 | 0.878 | 1.745 | 1.217 | −0.750 | 1.463 |
| GPR26-Tango | 1.012 | 0.799 | 0.978 | 1.117 | 1.167 | 0.665 | −0.124 | 1.031 |
| GPR27-Tango | −0.495 | −0.495 | −0.495 | 0.853 | 0.133 | −0.495 | −1.369 | 0.368 |
| GPR3-Tango | −0.125 | 0.587 | 0.105 | 0.170 | 0.211 | −0.086 | −1.328 | −0.164 |
| GPR31-Tango | −0.227 | 0.847 | −0.129 | 0.386 | −0.427 | −0.804 | −1.682 | −0.804 |
| GPR32-Tango | 1.045 | 1.231 | 1.557 | 1.571 | 1.308 | 0.872 | −2.099 | 0.388 |
| GPR34-Tango | 0.520 | 0.765 | 1.270 | 0.904 | 0.871 | 0.476 | −1.109 | 0.924 |
| GPR35-Tango | 0.571 | 0.458 | 0.849 | 0.788 | 0.544 | 0.317 | −1.840 | 0.201 |
| GPR37-Tango | 0.329 | 0.510 | 0.696 | 0.523 | 0.323 | 0.239 | −0.685 | 0.299 |
| GPR37L1-Tango | 0.372 | 0.780 | 0.525 | 0.755 | 0.287 | 0.081 | 0.146 | 0.403 |
| GPR39-Tango | 0.304 | 0.672 | 0.267 | 0.304 | 0.672 | 0.459 | 0.087 | 0.629 |
| GPR4-Tango | 0.136 | 0.352 | 0.426 | 0.406 | 0.128 | −0.190 | −1.001 | −0.459 |
| GPR44-Tango | 0.439 | 0.205 | 0.544 | 0.872 | 0.702 | −0.075 | −2.298 | 0.205 |
| GPR45-Tango | 0.149 | 0.206 | 0.468 | 0.275 | 0.275 | −0.059 | −1.282 | −0.339 |
| GPR50-Tango | 0.832 | 0.291 | 1.000 | 0.657 | 0.774 | 0.389 | −0.239 | 0.882 |
| GPR52-Tango | 0.544 | 0.830 | 0.188 | 0.761 | −0.082 | −0.216 | −0.263 | −0.337 |
| GPR55-Tango | −0.221 | 0.600 | 0.383 | 0.628 | 0.204 | −0.232 | −0.477 | −0.017 |
| GPR56-Tango | 0.317 | 0.500 | 0.852 | 0.437 | 0.631 | 0.166 | −0.786 | −0.116 |
| GPR6-Tango | 0.065 | 0.825 | 0.226 | 0.443 | 0.582 | 0.443 | −0.859 | 0.306 |
| GPR61-Tango | −0.335 | −0.123 | 0.019 | −0.484 | −0.267 | −0.267 | −1.703 | −0.296 |
| GPR62-Tango | 0.766 | 1.000 | 1.485 | 1.485 | −0.152 | 0.485 | −0.737 | 0.138 |
| GPR63-Tango | 0.000 | 0.369 | −0.018 | −0.066 | −0.152 | −0.341 | −0.927 | 0.000 |
| GPR64-Tango | −0.270 | −0.021 | −0.087 | −0.245 | −0.346 | −0.254 | −1.869 | −0.019 |
| GPR65-Tango | −0.183 | 0.033 | −0.117 | 0.103 | −0.155 | −0.112 | −0.974 | −0.333 |
| GPR68-Tango | −0.056 | 0.044 | 0.100 | 0.165 | −0.063 | −0.139 | −1.255 | −0.396 |
| GPR75- Tango | 0.007 | −0.113 | 0.220 | −0.127 | −0.192 | −0.114 | −1.559 | −0.823 |
| GPR77-Tango | −0.452 | 0.346 | 0.578 | −0.004 | 0.079 | −0.219 | −1.423 | −0.736 |
| GPR78-Tango | −0.257 | 0.682 | −0.177 | −0.018 | −0.257 | 0.158 | −1.856 | −0.018 |
| GPR82-Tango | −0.182 | 0.368 | 0.302 | 0.223 | 0.299 | −0.074 | −1.033 | −0.060 |
| GPR83-Tango | −0.011 | 0.265 | 0.360 | 0.378 | −0.235 | 0.172 | −1.772 | −0.533 |
| GPR84-Tango | 0.028 | 0.392 | 0.265 | 0.164 | 0.366 | −0.241 | −1.468 | 0.092 |
| GPR85-Tango | 0.344 | 0.459 | 0.650 | 0.784 | 0.750 | 0.412 | −0.961 | 0.079 |
| GPR87-Tango | −0.040 | 0.301 | 0.219 | 0.280 | 0.299 | −0.075 | −1.398 | −0.054 |
| GPR88-Tango | −0.339 | 0.307 | −0.726 | −0.841 | −0.391 | −0.481 | −2.225 | −1.256 |
| GPR97-Tango | 0.499 | 1.091 | 0.960 | 0.676 | 0.789 | 0.334 | −0.257 | 0.271 |
| GPRC5A-Tango | 0.067 | 0.288 | 0.060 | 0.267 | −0.444 | −0.478 | −1.348 | −0.723 |
| GPRC5B-Tango | −0.186 | 0.247 | −0.097 | 0.050 | −0.279 | −0.389 | −1.165 | −0.798 |
| GPRC5C-Tango | 0.423 | −0.030 | 0.351 | 0.308 | 0.010 | 0.117 | −1.418 | −0.036 |
| GPRC5D-Tango | 0.294 | 0.419 | 0.458 | 0.181 | 0.277 | −0.193 | −1.270 | 0.273 |
| GPRC6A-Tango | −0.253 | −0.044 | −0.204 | 0.013 | −0.375 | −0.340 | −1.818 | −0.705 |
| GRM7-Tango | −0.274 | −0.245 | −0.161 | −0.049 | −0.283 | −0.267 | −0.881 | −0.498 |
| GRM8-Tango | 0.089 | 0.086 | 0.089 | 0.469 | −0.136 | −0.055 | −1.314 | −0.529 |
| GRPR-Tango | 0.290 | −0.532 | 0.567 | 0.214 | 0.249 | −0.248 | −2.312 | 0.036 |

| | | | | | | | Bifidobac- | Bifidobac- |
|---|---|---|---|---|---|---|---|---|
| PRESTO-Tango | Collinsella | Collinsella | Collinsella | Collinsella | Collinsella | Collinsella | terium | terium |
| with Gifu Medium | aerofaciens-1# | aerofaciens-2# | aerofaciens-3# | stercoris-1# | stercoris-2# | stercoris-3# | spp | UC |
| GPR1-Tango | 1.226 | 1.484 | 0.882 | 0.184 | 0.052 | 0.267 | −0.335 | −0.215 |
| GPR101-Tango | 1.008 | 0.900 | 0.877 | 0.232 | 0.205 | 0.221 | −0.251 | 0.330 |
| GPR110-Tango | 1.126 | 1.135 | 0.851 | 0.489 | 0.211 | 0.308 | −1.123 | −0.053 |
| GPR111-Tango | 0.571 | 0.720 | 1.246 | 0.252 | 0.599 | 0.373 | 0.571 | 0.373 |
| GPR113-Tango | 0.104 | 0.448 | 0.303 | −0.542 | −0.307 | −0.112 | −0.384 | 0.487 |
| GPR114-Tango | 0.247 | 0.515 | 0.796 | −0.298 | 0.399 | −0.420 | 0.247 | 0.399 |
| GPR115-Tango | 0.324 | 0.358 | 0.231 | 0.068 | −0.081 | −0.167 | −0.128 | −0.156 |
| GPR116-Tango | 0.672 | 0.663 | 0.587 | 0.115 | 0.344 | 0.168 | −0.704 | 0.296 |
| GPR119-Tango | 1.214 | 1.287 | 0.816 | −0.059 | 0.379 | 0.506 | −0.837 | 0.379 |
| GPR12-Tango | 0.574 | 0.679 | 0.408 | 0.292 | 0.338 | 0.280 | −1.277 | −0.743 |
| GPR120-Tango | 0.491 | 0.598 | 0.314 | 1.112 | 0.878 | 0.878 | 0.112 | 0.491 |
| GPR123-Tango | 1.596 | 1.732 | 1.365 | 0.214 | 0.099 | 0.214 | 0.982 | −0.316 |
| GPR124-Tango | 1.441 | 1.611 | 1.487 | −0.048 | 0.350 | 0.122 | −1.554 | 0.515 |
| GPR125-Tango | 0.287 | 0.247 | 0.105 | −0.042 | −0.126 | −0.129 | 0.466 | −0.010 |
| GPR126-Tango | 1.840 | 1.659 | 1.798 | 0.112 | 0.040 | 0.071 | −0.654 | 0.416 |
| GPR132-Tango | 0.208 | 0.301 | 0.008 | −0.175 | −0.094 | −0.209 | −2.009 | −0.274 |
| GPR133-Tango | 0.281 | 0.293 | 0.080 | 0.039 | −0.077 | 0.144 | −0.550 | −0.160 |
| GPR135-Tango | 0.255 | 0.290 | 0.205 | −0.490 | 0.074 | −0.327 | −0.846 | −1.618 |
| GPR141-Tango | 1.110 | 0.776 | 0.364 | −0.301 | 0.136 | −0.354 | −0.073 | −0.986 |
| GPR142-Tango | 1.228 | 1.463 | 1.261 | 0.509 | 0.472 | 0.791 | 1.136 | 0.228 |
| GPR143-Tango | 0.541 | 0.769 | 0.492 | 0.302 | 0.532 | 0.414 | −0.265 | −0.407 |
| GPR144-Tango | 0.139 | 0.031 | 0.173 | 0.104 | 0.031 | 0.031 | −0.154 | −0.560 |
| GPR146-Tango | −0.234 | 0.379 | 1.263 | 0.766 | 0.379 | 0.485 | −0.862 | −0.862 |
| GPR148-Tango | 0.896 | 0.896 | 1.066 | 0.274 | −0.104 | 0.381 | −0.339 | 0.274 |
| GPR149-Tango | 1.398 | 1.503 | 1.445 | 0.380 | 0.652 | 0.546 | 1.674 | 0.203 |
| GPR15-Tango | 0.861 | 0.822 | 0.707 | −0.381 | 0.000 | 0.000 | −1.878 | −1.381 |
| GPR150-Tango | 0.107 | 0.387 | 0.823 | −2.115 | 0.387 | 0.387 | −2.115 | −2.115 |
| GPR151-Tango | 1.260 | 1.159 | 0.797 | 0.111 | 0.487 | 0.169 | −0.076 | −0.513 |
| GPR152-Tango | 0.531 | 0.617 | 0.579 | 0.241 | 0.356 | 0.404 | 1.032 | −0.358 |
| GPR153-Tango | 0.303 | 0.525 | 0.161 | 0.143 | 0.117 | 0.174 | −0.223 | −0.215 |
| GPR156-Tango | 1.327 | 1.457 | 1.397 | 0.345 | 0.677 | 0.783 | −0.609 | −0.858 |
| GPR157-Tango | 0.888 | 1.598 | 1.541 | 0.384 | 0.475 | −0.039 | −0.243 | 0.082 |
| GPR158-Tango | 1.326 | 1.194 | 0.578 | −0.071 | 0.039 | −0.259 | −0.780 | −0.102 |
| GPR160-Tango | 0.681 | 0.729 | 0.613 | 0.077 | 0.152 | 0.086 | −1.803 | −0.416 |
| GPR161-Tango | 0.731 | 0.967 | 0.586 | −0.105 | −0.038 | −0.149 | −1.908 | −0.255 |
| GPR162-Tango | 0.660 | 0.193 | 0.565 | 0.012 | 0.372 | −0.062 | −1.371 | −1.087 |
| GPR17-Tango | 0.526 | 0.443 | 0.475 | 0.087 | 0.033 | −0.007 | 0.181 | −0.215 |
| GPR171-Tango | 0.957 | 1.042 | 0.982 | 0.445 | 0.858 | 0.779 | 0.009 | −0.136 |
| GPR173-Tango | 1.000 | 0.896 | 1.000 | 0.783 | −0.104 | 0.274 | 0.896 | 1.066 |
| GPR174-Tango | 1.000 | 1.645 | 1.149 | 0.645 | 0.864 | 0.469 | −0.136 | −0.076 |
| GPR18-Tango | 1.303 | 1.394 | 1.199 | 1.051 | 1.143 | 1.114 | 0.239 | 0.262 |
| GPR182-Tango | 0.657 | 0.541 | 0.146 | 0.376 | −0.043 | −0.142 | −1.356 | −0.154 |
| GPR183-Tango | 0.340 | 0.540 | 0.280 | 0.114 | −0.139 | 0.340 | −1.017 | −0.709 |
| GPR19-Tango | 1.246 | 1.346 | 0.977 | 0.541 | 0.639 | 0.093 | −1.689 | −0.631 |
| GPR20-Tango | 0.395 | 0.512 | 0.333 | −0.545 | −0.263 | 0.333 | −1.054 | −1.667 |
| GPR21-Tango | 1.170 | 1.982 | 1.585 | 1.433 | 1.263 | 1.104 | −0.234 | 0.485 |
| GPR22-Tango | 1.447 | 1.360 | 1.183 | 0.377 | 0.070 | 0.195 | 0.583 | 0.273 |
| GPR25-Tango | 0.598 | 1.282 | 1.463 | 1.217 | 0.314 | −0.402 | −0.122 | 0.491 |
| GPR26-Tango | 0.822 | 1.296 | 0.838 | 0.765 | 0.808 | 0.640 | 0.716 | 0.335 |
| GPR27-Tango | 1.000 | 2.133 | 1.538 | −2.369 | −0.495 | 0.133 | 0.569 | 0.133 |
| GPR3-Tango | 0.611 | 0.682 | 0.842 | 0.003 | −0.034 | 0.266 | −0.400 | −0.136 |
| GPR31-Tango | 1.341 | 1.240 | 1.057 | −0.333 | −0.487 | −0.227 | −0.882 | −0.427 |
| GPR32-Tango | 1.686 | 1.539 | 1.571 | 0.472 | 1.431 | 0.939 | −0.684 | −0.914 |
| GPR34-Tango | 1.326 | 1.255 | 1.520 | 0.570 | 0.924 | 1.067 | −0.714 | −0.405 |
| GPR35-Tango | 1.102 | 1.211 | 0.798 | 0.575 | 0.505 | 0.523 | −0.355 | −0.299 |
| GPR37-Tango | 0.759 | 0.851 | 0.713 | 0.531 | 0.314 | 0.262 | −0.666 | 0.268 |
| GPR37L1-Tango | 0.967 | 0.967 | 0.802 | 0.487 | 0.727 | 0.776 | 0.287 | 0.132 |
| GPR39-Tango | 1.087 | 0.755 | 0.714 | 1.000 | 0.150 | 0.672 | 0.555 | −0.046 |
| GPR4-Tango | 0.807 | 0.908 | 0.834 | 0.518 | 0.082 | 0.009 | −0.937 | −0.101 |
| GPR44-Tango | 0.950 | 1.072 | 0.327 | 0.205 | 0.609 | −0.561 | −0.561 | −0.182 |
| GPR45-Tango | 1.049 | 1.071 | 0.863 | 0.513 | 0.317 | 0.468 | −2.000 | −1.059 |
| GPR50-Tango | 1.263 | 1.050 | 1.225 | 0.553 | 0.919 | 0.851 | −0.365 | −0.409 |
| GPR52-Tango | 1.030 | 1.503 | 0.885 | 0.544 | 0.544 | −0.263 | 0.097 | 0.134 |
| GPR55-Tango | 0.589 | 0.738 | 0.692 | 0.267 | 0.121 | 0.285 | −0.267 | 0.072 |
| GPR56-Tango | 1.424 | 1.326 | 1.276 | 0.394 | 0.492 | 0.477 | −1.320 | 0.183 |
| GPR6-Tango | 1.750 | 1.203 | 0.983 | 0.844 | 0.504 | 0.443 | −0.659 | −0.819 |
| GPR61-Tango | 0.704 | 0.571 | 0.216 | −0.199 | −0.329 | −0.111 | −0.819 | −0.418 |
| GPR62-Tango | 0.766 | 0.766 | 0.766 | −0.152 | 1.000 | 0.766 | 0.000 | 0.138 |
| GPR63-Tango | 0.461 | 0.368 | 0.256 | 0.033 | 0.103 | 0.011 | −1.029 | −0.092 |
| GPR64-Tango | 0.333 | 0.198 | 0.123 | −0.272 | −0.224 | −0.229 | 0.073 | −0.149 |
| GPR65-Tango | 0.314 | 0.411 | 0.150 | −0.031 | −0.130 | 0.069 | −0.254 | 0.018 |
| GPR68-Tango | 0.706 | 0.676 | 0.463 | 0.004 | 0.159 | −0.014 | −0.150 | −0.261 |
| GPR75- Tango | 0.532 | 0.651 | 0.503 | −0.472 | 0.199 | −0.067 | −1.447 | −0.926 |
| GPR77-Tango | 1.229 | 0.741 | 0.845 | 0.312 | 0.340 | 0.291 | −1.423 | −0.111 |
| GPR78-Tango | 0.331 | 0.731 | 0.158 | −0.064 | −0.177 | 0.061 | −0.458 | 0.256 |
| GPR82-Tango | 0.789 | 1.098 | 0.832 | 0.220 | −0.068 | 0.015 | −1.410 | −0.174 |
| GPR83-Tango | 0.662 | 0.750 | 0.515 | 0.241 | 0.145 | 0.092 | −1.979 | −0.642 |
| GPR84-Tango | 0.482 | 0.619 | 0.476 | 0.215 | 0.171 | −0.052 | −0.021 | 0.174 |
| GPR85-Tango | 0.731 | 0.960 | 0.722 | 0.356 | 0.447 | 0.630 | −0.652 | 0.023 |
| GPR87-Tango | 0.457 | 0.601 | 0.582 | 0.213 | 0.294 | 0.258 | 0.220 | 0.007 |
| GPR88-Tango | 0.636 | 0.799 | 0.636 | −0.539 | −0.163 | 0.147 | −1.941 | −0.841 |
| GPR97-Tango | 2.293 | 2.076 | 1.825 | 0.789 | 0.789 | 0.960 | 1.211 | −0.501 |
| GPRC5A-Tango | 0.992 | 1.146 | 0.950 | −0.159 | 0.004 | −0.090 | 0.137 | −0.421 |
| GPRC5B-Tango | 1.048 | 1.194 | 0.988 | 0.369 | 0.097 | 0.147 | −0.498 | −0.699 |
| GPRC5C-Tango | 0.949 | 0.666 | 0.409 | 0.018 | 0.253 | 0.325 | −1.527 | −0.741 |
| GPRC5D-Tango | 0.681 | 0.904 | 0.819 | 0.614 | 0.369 | 0.239 | 0.639 | −0.245 |
| GPRC6A-Tango | 0.137 | 0.165 | −0.016 | −0.008 | −0.213 | −0.082 | −1.953 | −0.369 |
| GRM7-Tango | 0.409 | 0.334 | 0.141 | −0.267 | −0.198 | −0.367 | −0.783 | −0.445 |
| GRM8-Tango | 0.587 | 0.759 | 0.371 | −0.081 | 0.123 | 0.019 | −0.145 | −0.153 |
| GRPR-Tango | 0.363 | 0.590 | 0.606 | 0.138 | 0.342 | 0.363 | −1.326 | −0.930 |

| PRESTO-Tango | Bifidobacterium | Bifidobacterium | Bifidobacterium | Morganella | Morganella | Morganella | Morganella | Morganella |
|---|---|---|---|---|---|---|---|---|
| with Gifu Medium | adolescentis-1# | adolescentis-2# | adolescentis-3# | spp-1# | spp-2# | spp-3# | spp-4# | spp-5# |
| GPR1-Tango | 0.431 | 0.283 | −0.823 | 0.374 | 0.509 | 0.594 | 0.487 | 0.573 |
| GPR101-Tango | 0.465 | 0.420 | 0.332 | 0.881 | 0.359 | 0.553 | 0.503 | 0.459 |
| GPR110-Tango | 0.203 | 0.130 | 0.130 | 0.736 | −0.062 | 0.340 | 0.300 | 0.287 |
| GPR111-Tango | 0.308 | 0.543 | 0.436 | 0.695 | 0.856 | 1.000 | 1.216 | 1.420 |
| GPR113-Tango | −0.048 | −0.195 | 1.101 | −0.149 | 0.007 | 0.007 | 0.135 | 0.681 |
| GPR114-Tango | 0.325 | 0.446 | −0.082 | −0.082 | 0.399 | 0.580 | 0.902 | 1.077 |
| GPR115-Tango | −0.138 | −0.145 | −0.024 | −0.300 | −0.286 | −0.141 | −0.171 | −0.227 |
| GPR116-Tango | 0.407 | 0.476 | 0.365 | 0.685 | 0.664 | 0.576 | 0.641 | 0.674 |
| GPR119-Tango | 0.816 | −0.152 | 0.566 | 0.506 | 0.623 | 0.379 | 1.176 | 0.941 |
| GPR12-Tango | 0.401 | 0.924 | 0.554 | 0.590 | 0.408 | 0.760 | 0.419 | 0.751 |
| GPR120-Tango | 0.491 | 0.745 | 0.598 | 0.878 | 1.623 | 0.112 | 1.112 | 0.598 |
| GPR123-Tango | 1.702 | 1.596 | 0.958 | 0.312 | −0.316 | −0.033 | 0.110 | 0.259 |
| GPR124-Tango | 0.805 | 1.791 | 0.927 | 0.611 | 0.312 | 0.312 | 0.312 | 0.077 |
| GPR125-Tango | −0.007 | −0.197 | 0.043 | 0.041 | −0.326 | −0.159 | −0.271 | −0.277 |
| GPR126-Tango | 0.259 | 0.678 | 0.384 | 0.858 | 0.715 | 0.698 | 0.769 | 0.882 |
| GPR132-Tango | 0.098 | −0.005 | −0.394 | −0.145 | −0.311 | −0.301 | −0.327 | −0.273 |
| GPR133-Tango | −0.055 | 0.080 | −0.190 | −0.033 | −0.134 | −0.122 | −0.095 | −0.133 |
| GPR135-Tango | −0.460 | −0.181 | −0.327 | −0.521 | −0.885 | −0.411 | −0.181 | −0.490 |
| GPR141-Tango | 0.776 | 1.255 | 0.571 | 0.397 | −0.391 | 0.461 | 0.461 | 0.541 |
| GPR142-Tango | 1.026 | 1.000 | 0.112 | 0.760 | −0.368 | 0.791 | 0.355 | −0.368 |
| GPR143-Tango | 0.494 | 0.219 | −0.023 | −0.008 | −0.149 | −0.077 | 0.135 | 0.173 |
| GPR144-Tango | 0.139 | −0.154 | 0.173 | −0.367 | −0.417 | −0.072 | 0.486 | 0.608 |
| GPR146-Tango | −0.234 | 0.888 | −0.234 | 0.000 | 0.379 | 0.485 | 1.104 | 0.485 |
| GPR148-Tango | 0.381 | −0.104 | 0.274 | 1.066 | 0.097 | −0.339 | 0.661 | 0.528 |
| GPR149-Tango | 2.438 | 2.645 | 2.333 | 1.034 | 1.308 | 1.017 | 0.363 | 0.791 |
| GPR15-Tango | −0.506 | 0.034 | 0.089 | −0.458 | −0.878 | −0.293 | −0.816 | 0.320 |
| GPR150-Tango | 0.621 | 0.387 | 1.000 | 0.823 | 0.387 | 0.387 | −0.241 | −0.241 |
| GPR151-Tango | 0.760 | 1.472 | 1.595 | 0.531 | −0.343 | −0.011 | 0.260 | −0.343 |
| GPR152-Tango | 0.327 | 0.925 | 0.612 | −0.348 | −0.139 | 0.097 | −0.110 | 0.057 |
| GPR153-Tango | −0.013 | 0.156 | −0.001 | 0.204 | −0.016 | −0.290 | 0.026 | 0.004 |
| GPR156-Tango | 0.904 | 0.939 | 0.939 | 1.316 | 1.373 | 1.424 | 1.380 | 1.243 |
| GPR157-Tango | 0.311 | 0.384 | 0.248 | 0.311 | 0.710 | 0.419 | 1.000 | 1.221 |
| GPR158-Tango | 1.723 | 1.942 | 1.015 | 0.310 | −0.605 | −0.190 | −0.295 | 0.194 |
| GPR160-Tango | 0.150 | 0.393 | 0.254 | −0.260 | −0.405 | −0.207 | −0.328 | −0.145 |
| GPR161-Tango | −0.284 | 0.015 | −0.348 | 0.062 | −0.154 | −0.192 | −0.250 | 0.000 |
| GPR162-Tango | 1.036 | 0.409 | 0.059 | −0.222 | 0.193 | −0.573 | −0.140 | −1.087 |
| GPR17-Tango | 0.307 | 0.121 | −0.228 | 0.307 | 0.451 | 0.352 | 0.288 | 0.215 |
| GPR171-Tango | 0.696 | 1.059 | 0.666 | −0.097 | 0.493 | 0.817 | 1.018 | 0.991 |
| GPR173-Tango | −0.104 | 1.159 | 0.274 | 1.066 | 1.246 | 0.783 | 1.066 | 0.896 |
| GPR174-Tango | 1.054 | 0.737 | 0.222 | −0.263 | −0.379 | 0.509 | 0.509 | 0.509 |
| GPR18-Tango | 0.686 | 0.568 | 0.930 | 0.423 | 0.385 | 0.816 | 1.107 | 1.047 |
| GPR182-Tango | 0.705 | 0.722 | 0.777 | 0.715 | 0.282 | 0.738 | 0.718 | 0.639 |
| GPR183-Tango | −0.038 | 0.163 | 0.163 | −0.161 | −0.551 | −0.612 | −0.184 | −0.017 |
| GPR19-Tango | 0.823 | 1.225 | 0.837 | −0.118 | −0.443 | −0.184 | −0.297 | −0.095 |
| GPR20-Tango | 0.199 | −0.801 | −0.667 | −0.433 | −0.329 | 0.395 | 0.512 | 0.455 |
| GPR21-Tango | 1.585 | 1.104 | 1.170 | 0.632 | 1.263 | 1.766 | 2.087 | 1.982 |
| GPR22-Tango | 0.521 | 0.782 | 0.811 | 0.107 | −0.424 | 0.195 | 0.097 | 0.296 |
| GPR25-Tango | 1.813 | −0.122 | −0.402 | 0.598 | 1.813 | 1.217 | 1.000 | 1.545 |
| GPR26-Tango | 1.045 | 0.760 | 1.384 | 0.605 | 1.019 | 1.245 | 1.085 | 1.373 |
| GPR27-Tango | −0.784 | −0.495 | 0.133 | 0.569 | 0.133 | −0.495 | 0.746 | 0.569 |
| GPR3-Tango | 0.266 | 0.105 | −0.097 | 0.486 | 0.456 | 0.427 | 0.464 | 0.561 |
| GPR31-Tango | 0.933 | 0.258 | −0.082 | 0.543 | −0.333 | 0.676 | 0.649 | 1.276 |
| GPR32-Tango | −0.099 | −0.023 | −0.414 | 0.663 | 0.638 | 1.500 | 1.621 | 2.128 |
| GPR34-Tango | 0.829 | 0.787 | 0.520 | 0.476 | 1.444 | 0.962 | 1.640 | 1.444 |
| GPR35-Tango | −0.394 | −0.503 | 0.048 | 0.095 | 0.361 | 0.554 | 0.724 | 0.460 |
| GPR37-Tango | 0.506 | 0.465 | 0.505 | 0.594 | 0.491 | 0.729 | 0.612 | 0.518 |
| GPR37L1-Tango | 0.643 | 0.791 | 0.527 | 0.264 | 0.178 | 0.164 | 0.432 | 0.290 |
| GPR39-Tango | 0.672 | 0.358 | 1.087 | 0.672 | 0.459 | 0.209 | 0.267 | 0.629 |
| GPR4-Tango | 0.531 | 0.752 | 0.392 | 0.276 | −0.197 | 0.439 | 0.251 | 0.541 |
| GPR44-Tango | 0.205 | 2.236 | 1.527 | −0.075 | 0.072 | 1.327 | 0.702 | 0.205 |
| GPR45-Tango | 0.074 | 0.275 | −0.186 | 0.165 | 0.006 | 0.932 | 1.009 | 0.657 |
| GPR50-Tango | 1.314 | 1.000 | 0.851 | −0.365 | 0.319 | 0.155 | 0.774 | 1.034 |
| GPR52-Tango | 1.214 | 0.688 | 1.377 | 0.761 | 0.239 | 0.688 | 0.830 | 0.624 |
| GPR55-Tango | 0.526 | 0.231 | 0.582 | 0.306 | 0.555 | 0.458 | 0.428 | 0.396 |
| GPR56-Tango | 0.372 | 0.568 | 0.725 | 0.522 | 0.418 | 0.884 | 0.824 | 0.852 |
| GPR6-Tango | 1.266 | 1.185 | 0.994 | 1.309 | 1.065 | 1.457 | 0.951 | 1.115 |
| GPR61-Tango | 0.171 | −0.304 | −0.026 | −0.603 | −0.623 | −0.215 | −0.682 | −0.603 |
| GPR62-Tango | 2.104 | 1.632 | 1.000 | 1.202 | −0.737 | −0.152 | 0.766 | 0.485 |
| GPR63-Tango | 0.646 | 0.625 | −0.237 | 0.063 | −0.023 | 0.093 | 0.008 | −0.041 |
| GPR64-Tango | −0.254 | −0.063 | −0.030 | 0.155 | −0.041 | 0.001 | −0.152 | −0.038 |
| GPR65-Tango | −0.136 | 0.168 | −0.284 | −0.130 | −0.185 | −0.228 | −0.291 | −0.349 |
| GPR68-Tango | 0.120 | 0.216 | −0.104 | −0.453 | −0.702 | −0.347 | −0.428 | −0.358 |
| GPR75- Tango | −0.122 | 0.217 | −0.035 | −0.675 | −0.360 | −0.383 | −0.263 | 0.131 |
| GPR77-Tango | 0.774 | 0.645 | 0.543 | −0.071 | −0.198 | 0.451 | 0.192 | 0.871 |
| GPR78-Tango | 0.427 | 0.366 | 0.906 | −0.187 | −0.039 | 0.208 | −0.006 | −0.064 |
| GPR82-Tango | 0.179 | 0.625 | −0.491 | 0.895 | 0.206 | 0.717 | 0.672 | 0.657 |
| GPR83-Tango | −0.133 | −0.461 | −0.728 | −0.262 | −0.190 | −0.209 | −0.066 | −0.215 |
| GPR84-Tango | 0.018 | 0.144 | 0.145 | 0.255 | 0.359 | 0.272 | 0.555 | 0.296 |
| GPR85-Tango | 0.470 | 0.424 | 0.034 | 0.731 | 0.784 | 0.722 | 0.765 | 0.696 |
| GPR87-Tango | 0.089 | 0.219 | 0.095 | 0.169 | −0.041 | 0.173 | 0.351 | 0.325 |
| GPR88-Tango | 0.565 | 0.402 | 0.565 | −0.289 | −0.598 | −0.194 | 0.565 | 0.481 |
| GPR97-Tango | 1.023 | 1.149 | 1.651 | 0.334 | 1.170 | 1.636 | 1.371 | 1.454 |
| GPRC5A-Tango | 0.803 | 0.585 | 0.310 | 0.004 | −0.251 | 0.057 | −0.060 | −0.073 |
| GPRC5B-Tango | 0.103 | 0.727 | 0.615 | 0.005 | −0.465 | 0.001 | −0.316 | −0.245 |
| GPRC5C-Tango | −0.181 | −0.418 | −0.524 | −0.387 | −0.194 | 0.022 | −0.104 | −0.268 |
| GPRC5D-Tango | 0.158 | 0.472 | 0.910 | −0.014 | −0.352 | 0.158 | 0.128 | 0.164 |
| GPRC6A-Tango | −0.084 | −0.192 | −1.129 | −0.303 | −0.546 | −0.398 | −0.412 | −0.457 |
| GRM7-Tango | −0.107 | −0.073 | −0.311 | −0.703 | −0.572 | −0.589 | −0.367 | −0.528 |
| GRM8-Tango | −0.042 | 0.052 | −0.149 | 0.297 | 0.269 | 0.176 | 0.267 | 0.272 |
| GRPR-Tango | −0.566 | −0.947 | −1.371 | −0.886 | −0.153 | −0.383 | 0.363 | 0.170 |

| PRESTO-Tango | Morganella | (Enterobacteria) | (Enterobacteria) | (Enterobacteria) | (Enterobacteria) | (Enterobacteria) |
|---|---|---|---|---|---|---|
| with Gifu Medium | spp-6# | UC/UC-1# | UC/UC-2# | UC/UC-3# | UC/UC-4# | UC/UC-5# |
| GPR1-Tango | 0.361 | 0.727 | 0.371 | 0.632 | 0.406 | −2.054 |
| GPR101-Tango | 0.224 | 0.419 | 0.304 | 0.405 | 0.217 | −1.395 |
| GPR110-Tango | 0.415 | −0.316 | −0.341 | 0.267 | −0.117 | −1.813 |
| GPR111-Tango | 1.461 | −0.085 | −0.206 | 0.571 | 0.599 | −2.014 |
| GPR113-Tango | −0.090 | 0.303 | 0.085 | −0.384 | 0.236 | −2.069 |
| GPR114-Tango | 0.642 | −0.082 | −0.700 | 0.399 | 0.247 | −2.048 |
| GPR115-Tango | −0.351 | −0.130 | −0.336 | 0.025 | 0.063 | −2.158 |
| GPR116-Tango | 0.490 | 0.394 | −0.028 | 0.346 | 0.175 | −1.862 |
| GPR119-Tango | 1.070 | −0.322 | −0.556 | 0.111 | 0.310 | 1.322 |
| GPR12-Tango | 0.661 | 0.889 | 0.292 | 0.751 | 0.924 | −1.093 |
| GPR120-Tango | 0.314 | 0.112 | 1.112 | 0.745 | 0.745 | −0.402 |
| GPR123-Tango | −0.069 | −0.177 | −0.422 | 0.416 | 0.082 | −2.039 |
| GPR124-Tango | 0.458 | 0.047 | 0.350 | 0.206 | 0.312 | −1.082 |
| GPR125-Tango | −0.449 | −0.131 | −0.317 | −0.145 | −0.134 | −2.505 |
| GPR126-Tango | 0.939 | 0.333 | −0.035 | 0.359 | 0.208 | −0.629 |
| GPR132-Tango | −0.296 | −1.089 | −1.117 | −0.651 | −0.538 | −2.466 |
| GPR133-Tango | −0.387 | 0.026 | −0.221 | −0.004 | 0.134 | −2.238 |
| GPR135-Tango | −1.231 | −0.521 | −0.441 | −0.521 | −0.782 | −1.968 |
| GPR141-Tango | 0.397 | 0.397 | −0.104 | 0.342 | 0.522 | −1.851 |
| GPR142-Tango | 0.038 | −0.209 | 0.038 | −0.122 | 0.472 | −1.472 |
| GPR143-Tango | 0.184 | −0.461 | −0.654 | −0.317 | 0.019 | −2.068 |
| GPR144-Tango | −0.072 | 0.657 | −0.154 | 0.139 | 0.323 | −2.505 |
| GPR146-Tango | 0.485 | −1.737 | −0.515 | 0.202 | 0.485 | −0.515 |
| GPR148-Tango | 0.097 | 0.274 | 0.896 | −0.104 | 0.097 | −1.841 |
| GPR149-Tango | 0.280 | 1.345 | 0.830 | 0.652 | 0.757 | −0.518 |
| GPR15-Tango | −0.336 | −1.540 | −1.211 | −0.506 | −0.506 | −2.442 |
| GPR150-Tango | 0.621 | 0.387 | −1.115 | 0.107 | 0.107 | 1.000 |
| GPR151-Tango | −0.343 | 0.206 | −0.191 | 0.260 | −0.191 | −3.513 |
| GPR152-Tango | 0.102 | −0.991 | −0.509 | 0.090 | −0.086 | −2.181 |
| GPR153-Tango | 0.009 | −0.036 | 0.025 | 0.108 | 0.357 | −2.242 |
| GPR156-Tango | 1.085 | 0.921 | 0.952 | 0.797 | 1.014 | −0.797 |
| GPR157-Tango | 1.104 | 0.837 | 0.508 | 0.541 | 1.126 | −1.225 |
| GPR158-Tango | −0.435 | −0.224 | −0.010 | −0.135 | −0.435 | 0.123 |
| GPR160-Tango | −0.500 | −0.392 | −0.522 | 0.191 | 0.089 | −2.480 |
| GPR161-Tango | −0.483 | 0.001 | −0.377 | −0.051 | 0.011 | −2.141 |
| GPR162-Tango | −0.807 | 0.059 | 0.463 | −0.062 | 0.372 | −1.807 |
| GPR17-Tango | 0.349 | 0.410 | 0.267 | 0.562 | 0.319 | −1.620 |
| GPR171-Tango | 0.807 | −1.149 | −0.675 | 0.315 | 0.543 | −2.582 |
| GPR173-Tango | 0.274 | 1.159 | 0.097 | 1.159 | 1.528 | −0.967 |
| GPR174-Tango | 0.344 | −0.198 | −0.331 | −0.136 | 0.387 | −0.613 |
| GPR18-Tango | 0.831 | −0.037 | −0.297 | 0.637 | 1.014 | −1.553 |
| GPR182-Tango | 0.252 | 0.573 | 0.318 | 0.830 | 0.254 | −1.511 |
| GPR183-Tango | −0.297 | −0.837 | −0.612 | 0.217 | −0.161 | −1.582 |
| GPR19-Tango | −0.759 | −0.588 | −0.881 | 0.163 | −0.012 | −2.345 |
| GPR20-Tango | 0.753 | 0.078 | −0.801 | −0.433 | 0.874 | −1.667 |
| GPR21-Tango | 1.170 | −0.862 | 0.000 | 0.379 | 0.888 | 0.202 |
| GPR22-Tango | 0.040 | 0.528 | −0.177 | 0.080 | 0.369 | −1.424 |
| GPR25-Tango | 1.217 | 0.314 | 0.491 | 1.376 | 0.598 | 2.463 |
| GPR26-Tango | 1.252 | 0.860 | 0.854 | 1.005 | 0.998 | −1.012 |
| GPR27-Tango | 1.000 | −0.495 | −0.147 | 0.133 | 0.368 | 0.368 |
| GPR3-Tango | 0.192 | 0.927 | 0.726 | 0.880 | 0.995 | −1.755 |
| GPR31-Tango | 0.184 | 0.329 | −0.370 | 0.513 | −0.427 | −1.427 |
| GPR32-Tango | 1.550 | −0.142 | −0.099 | 1.183 | 1.332 | −1.684 |
| GPR34-Tango | 2.058 | 0.943 | 0.871 | 0.808 | 1.498 | −0.957 |
| GPR35-Tango | 0.652 | 0.223 | 0.168 | 0.679 | 1.177 | −1.916 |
| GPR37-Tango | 0.452 | 0.533 | 0.510 | 0.835 | 0.810 | −1.793 |
| GPR37L1-Tango | −0.029 | 0.192 | 0.019 | 0.403 | 0.425 | −1.630 |
| GPR39-Tango | 1.022 | −0.167 | 0.087 | 0.794 | 0.304 | −1.678 |
| GPR4-Tango | 0.112 | 0.267 | 0.178 | 0.370 | 0.153 | −2.142 |
| GPR44-Tango | 1.024 | 1.672 | 0.205 | 0.950 | 0.790 | −0.182 |
| GPR45-Tango | 0.538 | −0.206 | −0.384 | 0.750 | 0.513 | −2.121 |
| GPR50-Tango | 0.319 | 0.345 | 0.125 | 0.635 | 0.576 | −1.503 |
| GPR52-Tango | 0.503 | 0.796 | 1.030 | 0.544 | 0.503 | −0.020 |
| GPR55-Tango | 0.403 | −0.227 | −0.127 | 0.296 | 0.185 | −2.061 |
| GPR56-Tango | 0.614 | 0.495 | 0.486 | 0.340 | 0.481 | −1.640 |
| GPR6-Tango | 0.710 | 1.306 | 0.934 | 1.609 | 0.972 | −1.198 |
| GPR61-Tango | −0.766 | −0.682 | −0.593 | −0.071 | −0.839 | −2.534 |
| GPR62-Tango | −0.737 | 0.138 | −0.737 | 1.202 | −0.152 | −0.152 |
| GPR63-Tango | −0.012 | 0.325 | 0.055 | 0.001 | 0.309 | −2.288 |
| GPR64-Tango | −0.231 | −0.203 | −0.283 | −0.203 | 0.099 | −2.648 |
| GPR65-Tango | −0.389 | −0.328 | −0.220 | −0.250 | −0.128 | −2.278 |
| GPR68-Tango | −0.540 | −1.119 | −1.058 | −0.593 | −0.546 | −2.189 |
| GPR75- Tango | −0.149 | −0.200 | −0.258 | 0.114 | −0.010 | −2.571 |
| GPR77-Tango | 0.249 | 0.196 | 0.425 | 0.536 | 0.337 | −2.123 |
| GPR78-Tango | −0.243 | −0.089 | −0.257 | −0.124 | 0.006 | −2.076 |
| GPR82-Tango | 0.269 | 0.724 | 0.266 | 0.690 | 0.578 | −1.795 |
| GPR83-Tango | −0.345 | −0.430 | −0.569 | 0.124 | 0.165 | −2.626 |
| GPR84-Tango | 0.174 | 0.629 | 0.191 | 0.706 | 0.312 | −1.933 |
| GPR85-Tango | 0.582 | 0.650 | 0.298 | 0.600 | 0.968 | −1.844 |
| GPR87-Tango | 0.155 | 0.242 | 0.157 | 0.396 | 0.537 | −1.699 |
| GPR88-Tango | −0.444 | −0.661 | −0.794 | 0.432 | −0.240 | −2.048 |
| GPR97-Tango | 0.789 | 0.886 | 0.626 | 0.860 | 1.046 | −2.716 |
| GPRC5A-Tango | −0.265 | −0.603 | −0.729 | −0.037 | −0.093 | −2.340 |
| GPRC5B-Tango | −0.370 | −0.215 | −0.632 | 0.436 | −0.232 | −1.870 |
| GPRC5C-Tango | 0.149 | −0.505 | −0.488 | −0.257 | 0.222 | −2.427 |
| GPRC5D-Tango | 0.007 | −0.361 | −0.193 | 0.255 | 0.132 | −2.299 |
| GPRC6A-Tango | −0.571 | −0.583 | −0.541 | −0.257 | −0.222 | −2.582 |
| GRM7-Tango | −0.676 | −0.847 | −0.888 | −0.589 | −0.350 | −2.692 |
| GRM8-Tango | 0.297 | −0.696 | −0.596 | −0.096 | −0.162 | −2.567 |
| GRPR-Tango | −0.080 | −0.341 | −0.656 | 0.183 | 0.419 | −2.854 |

### Example 3. Human Gut Microbes Produce Compounds that Activate Aminergic Receptors

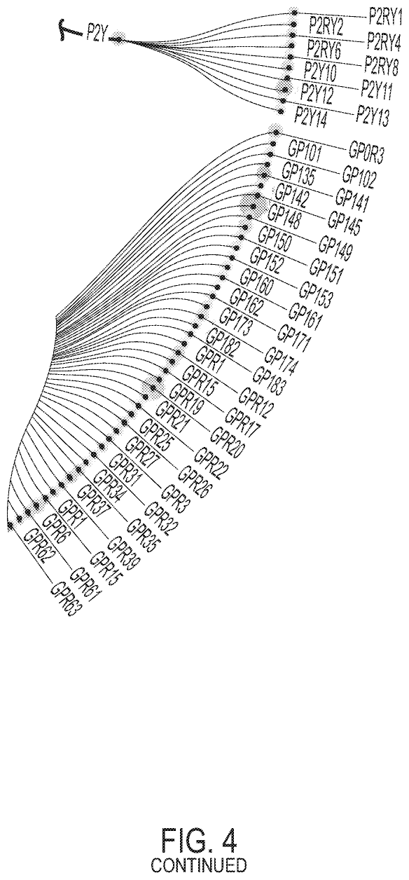

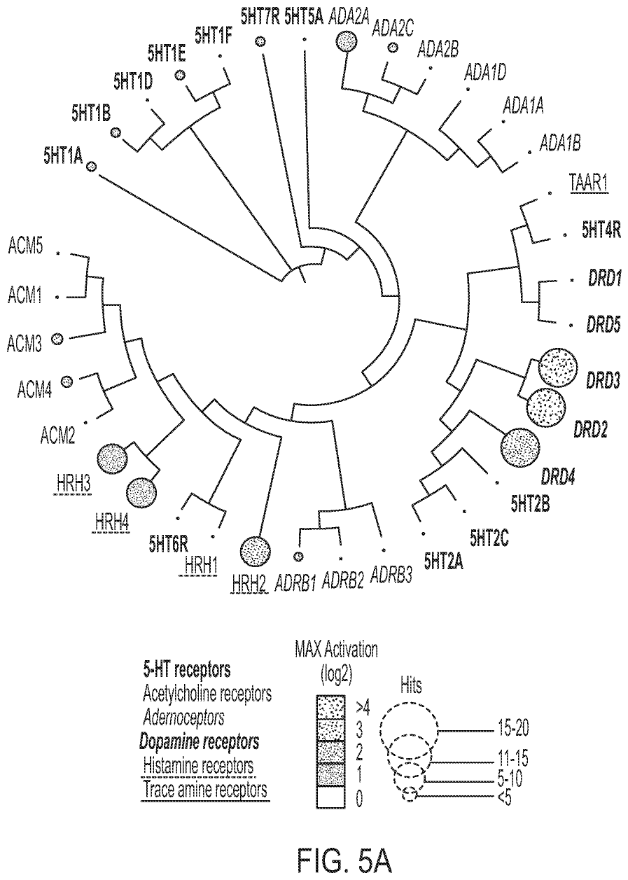

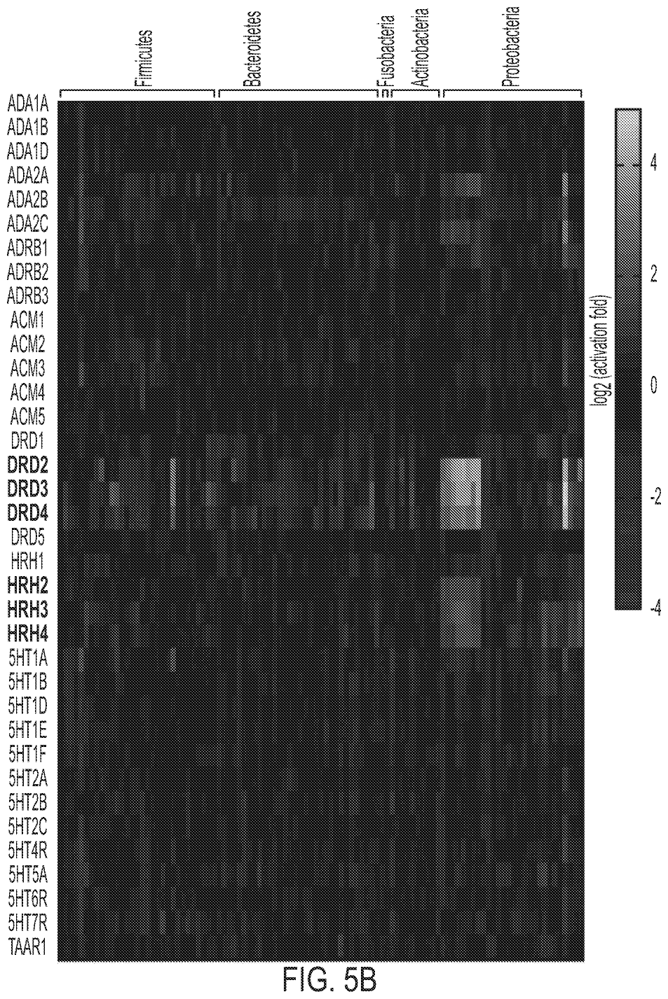

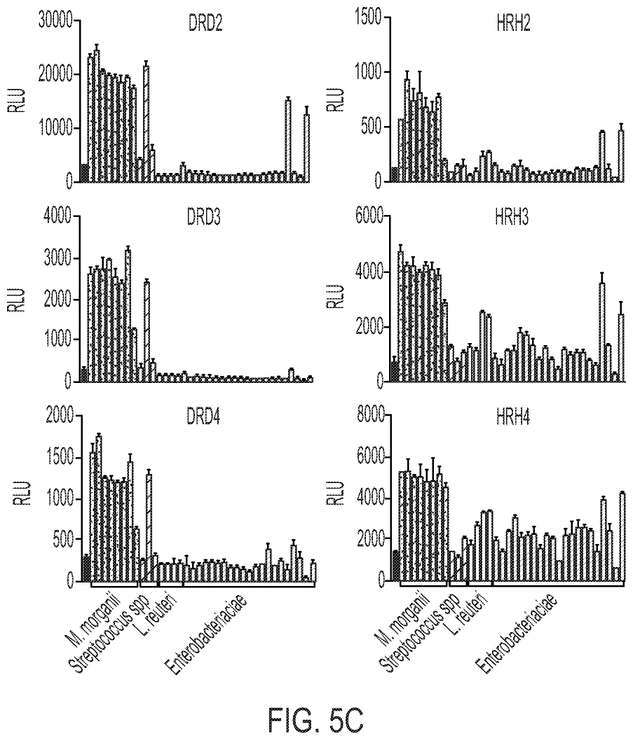

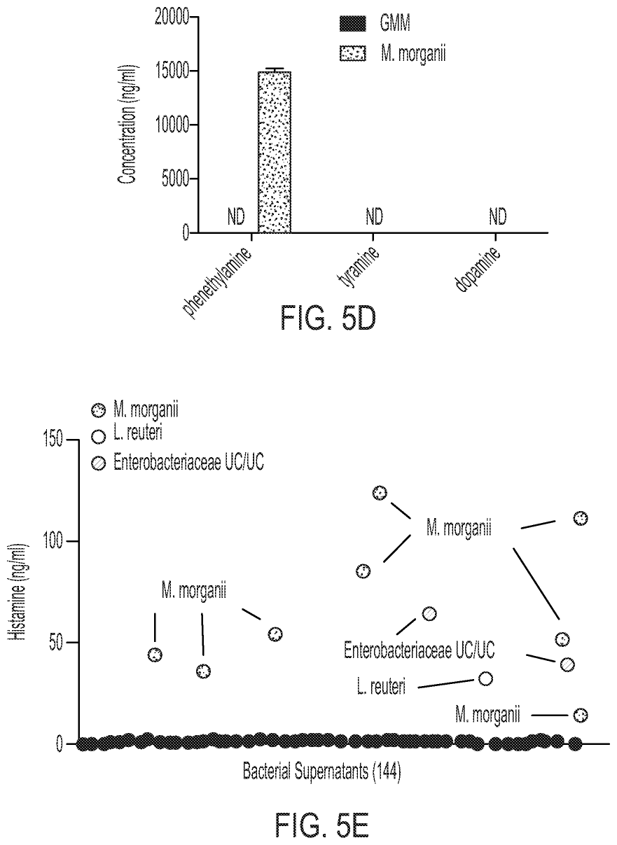

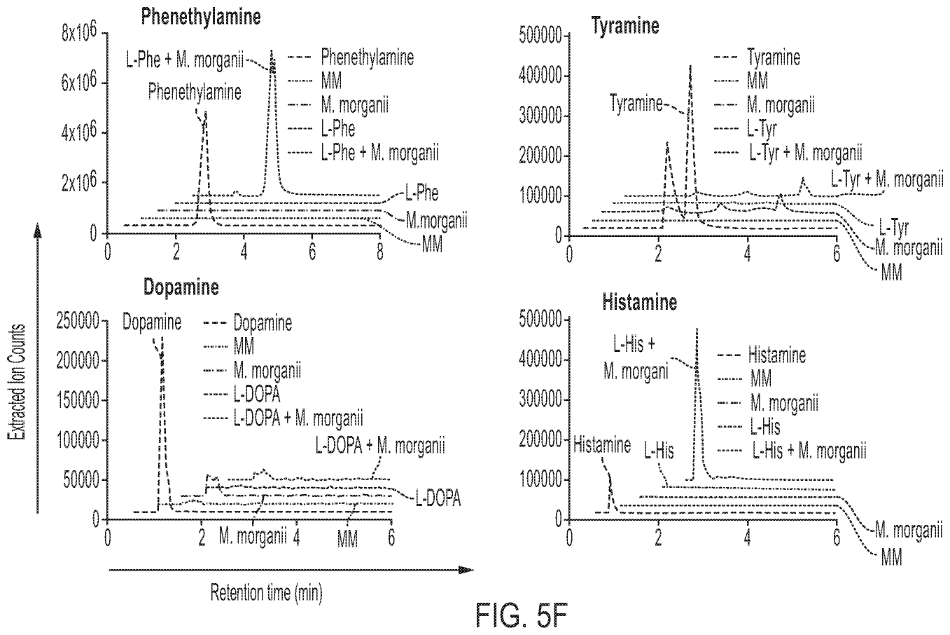

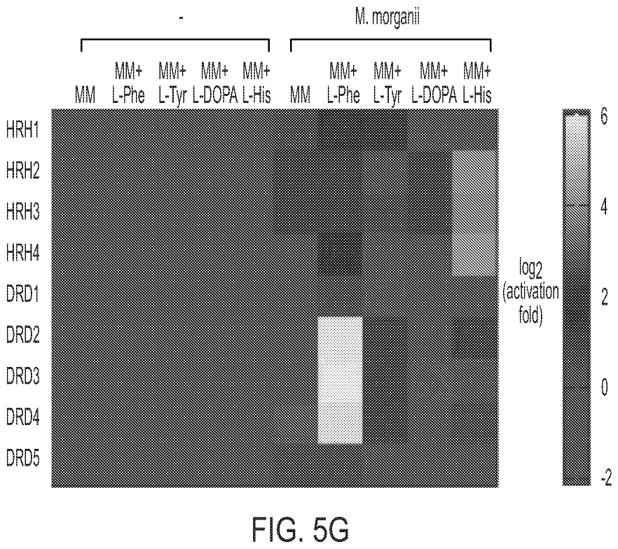

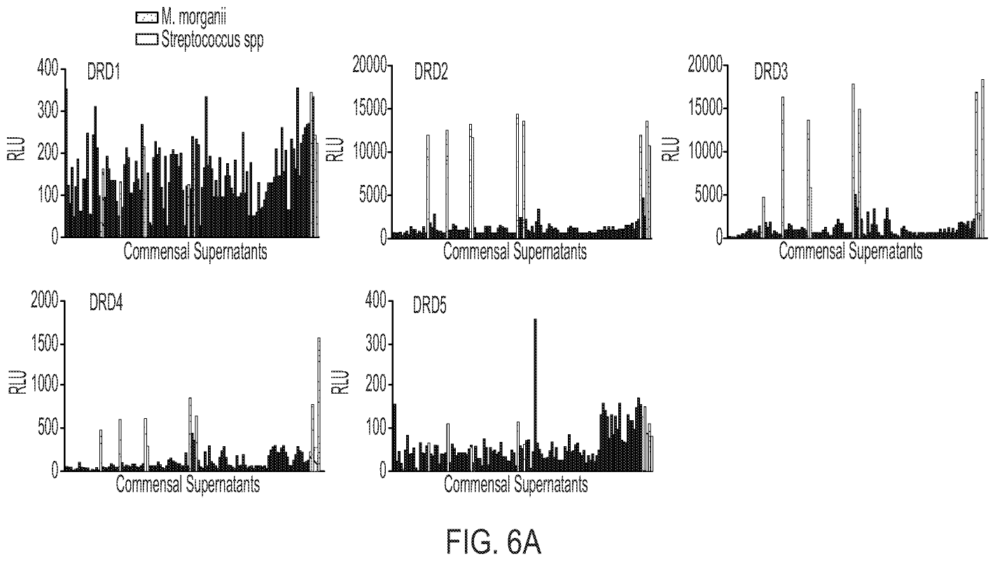

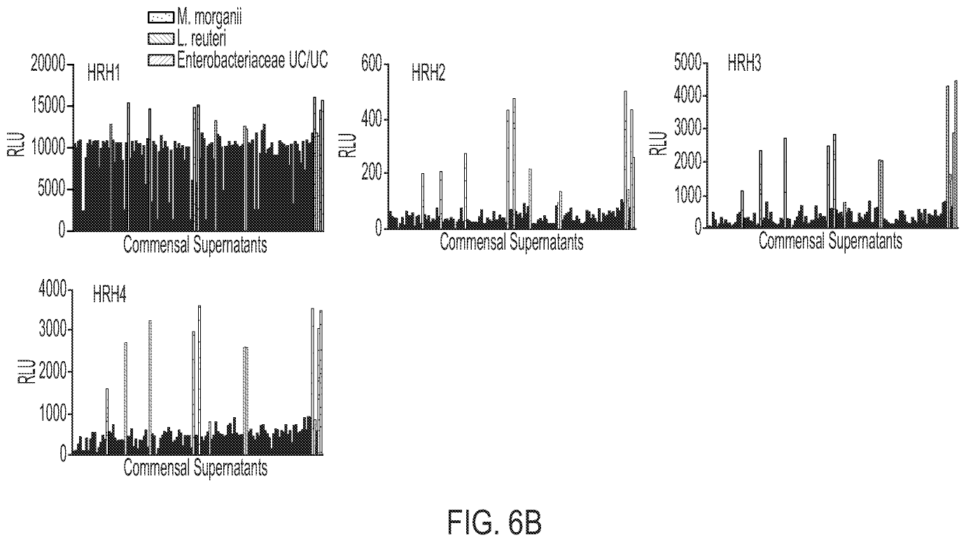

Besides the succinate receptor, the next most prevalent class of GPCRs activated by gut commensals was the aminergic receptors, which are expressed in diverse tissues and cell types and regulate a wide variety of core physiological processes ranging from neurotransmission to immunity. See, FIG. 4. (Albuquerque et al., 2009; Beaulieu and Gainetdinov, 2011; Thurmond et al., 2008). These hits included bacterial-derived activators of the dopamine (DRDs), histamine (HRHs) and adrenergic receptor families. More than a dozen commensal supernatants activated DRD2-4 or HRH2-4, as shown in FIGS. 5A, 6A and 6B. For example, ten strains from the phylum Proteobacteria activated both DRDs and HRHs, including eight strains from the species Morganella morganii. See, FIGS. 5B, 5C, 6A and 6B. The result suggests that production of DRD and HRH agonists is a conserved feature of M. morganii.

In contrast, two strains of L. reuteri that were found to activate HRHs, while two distinct strains of L. reuteri failed to activate HRHs despite displaying similar growth kinetics. See, FIGS. 5C, 6A and 6B. One strain of Streptococcus, but not two related isolates, activated DRD2-4, while two unclassified strains of Enterobacteriaceae activated HRH1-4 and DRD2 but failed to activate other DRDs. See FIGS. 5C, 6A and 6B.

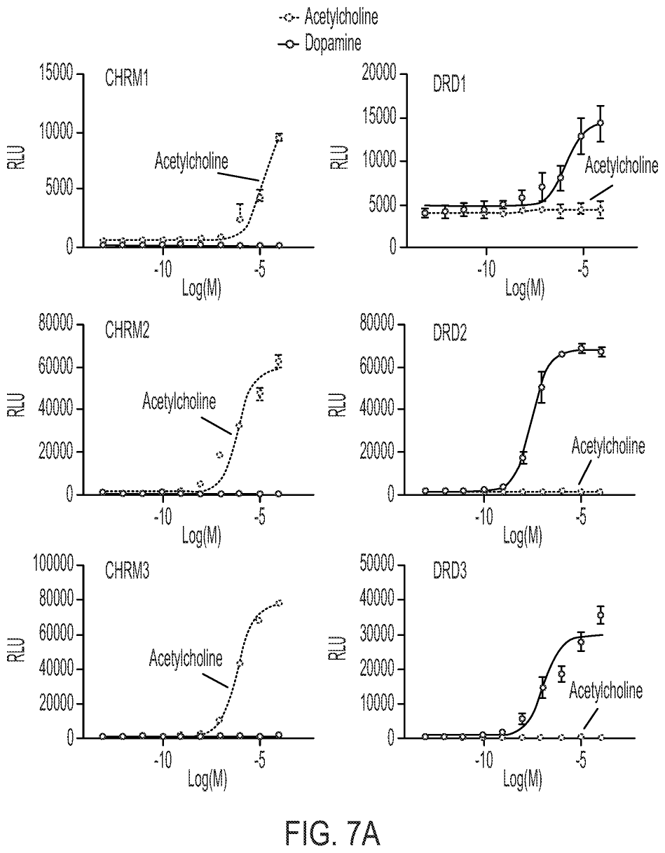

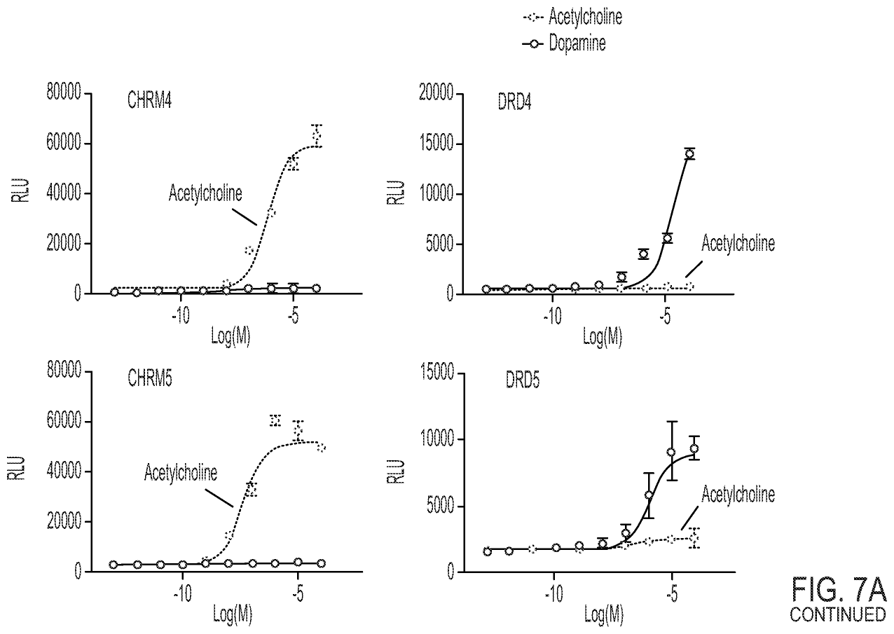

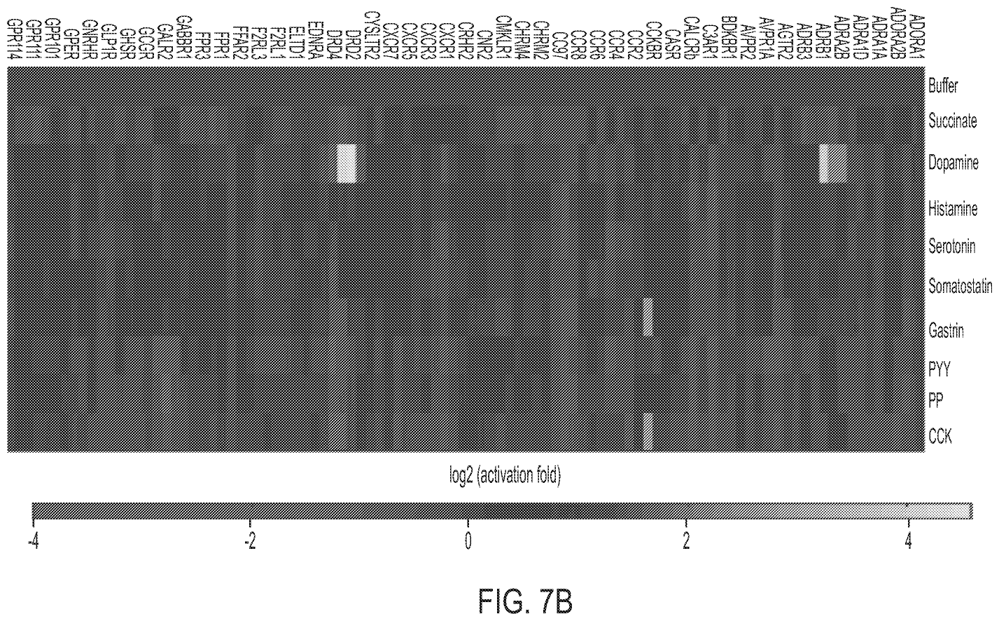

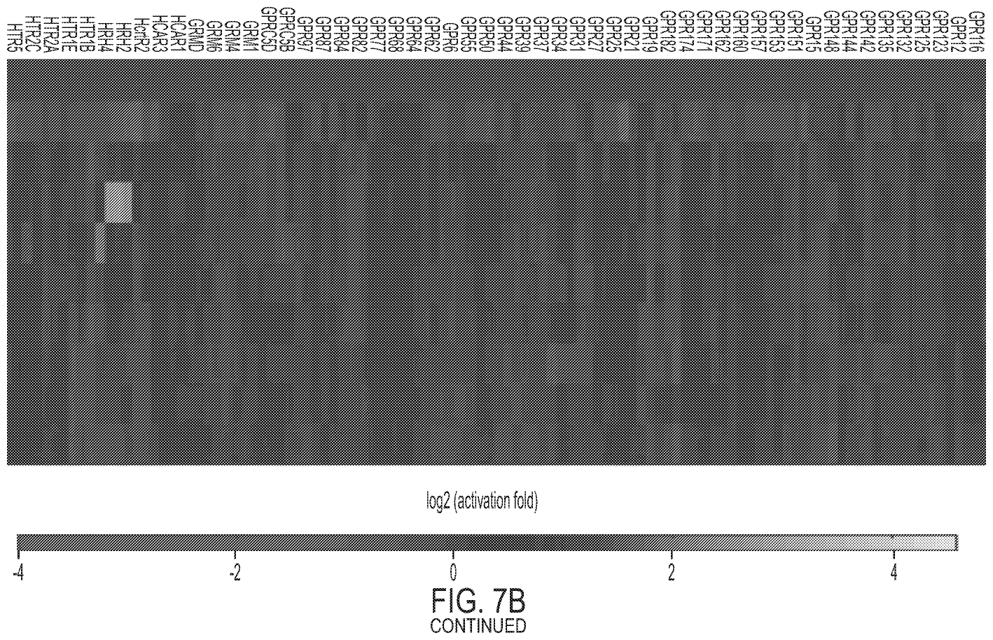

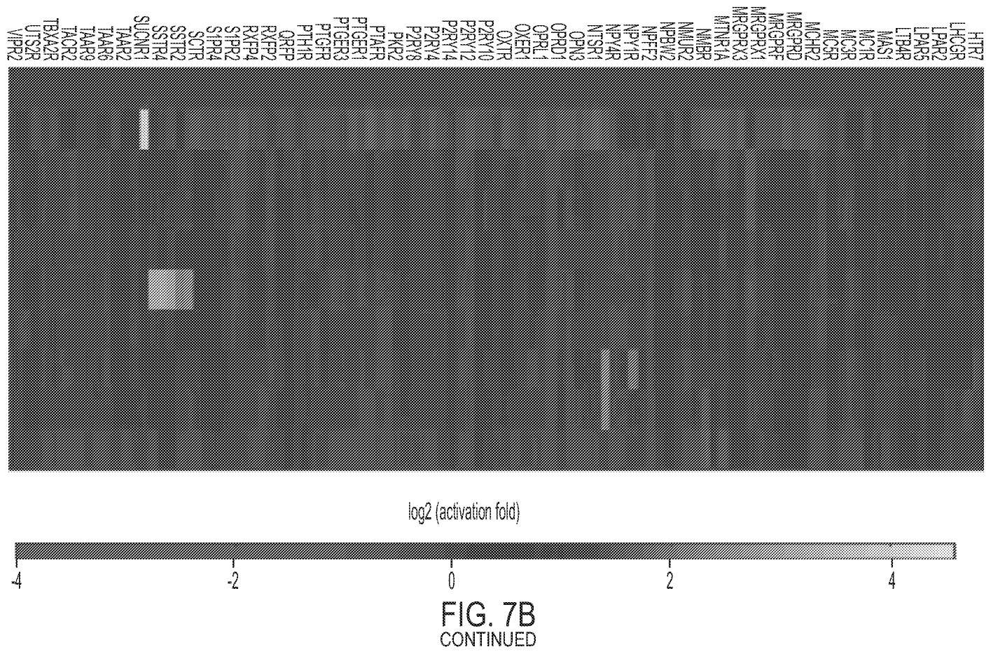

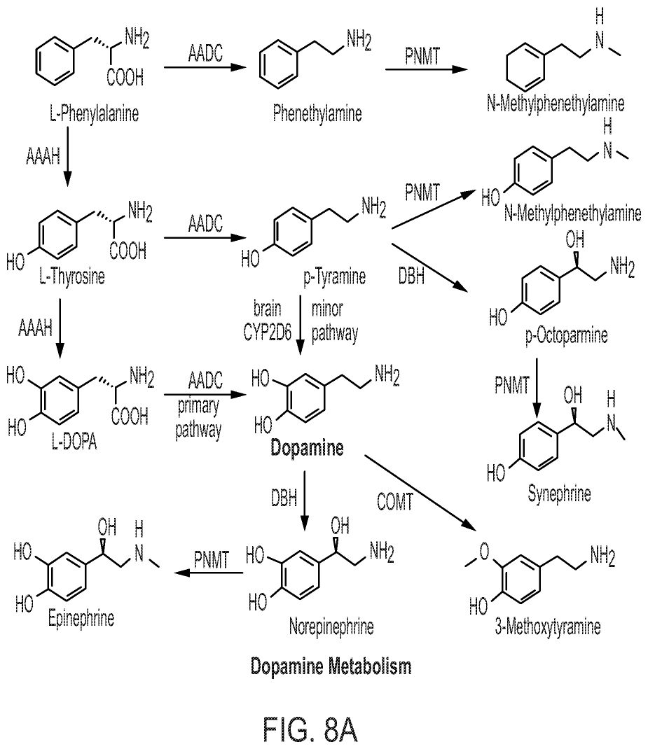

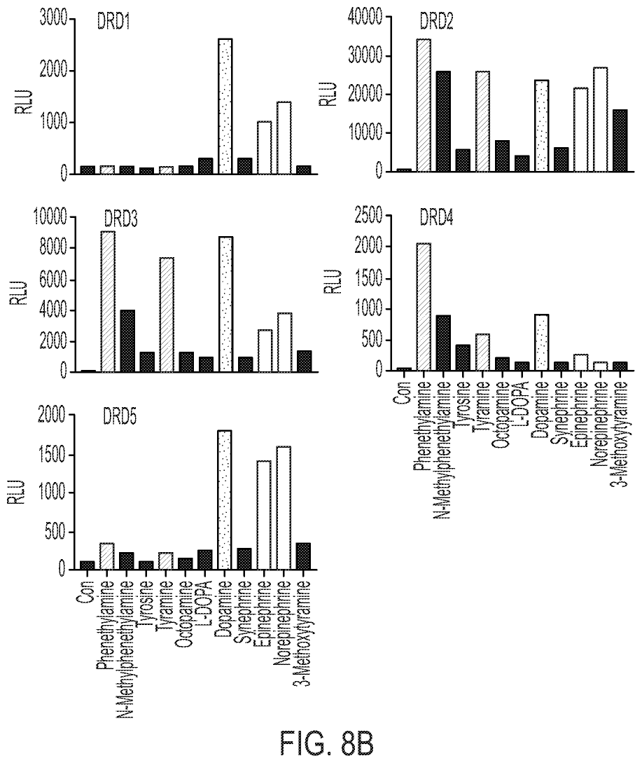

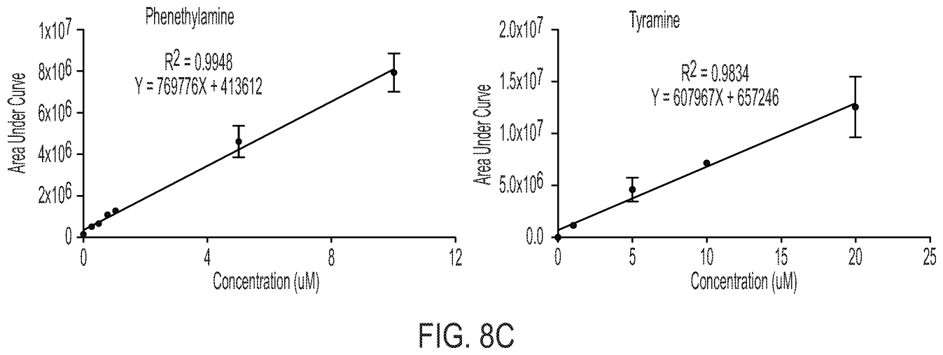

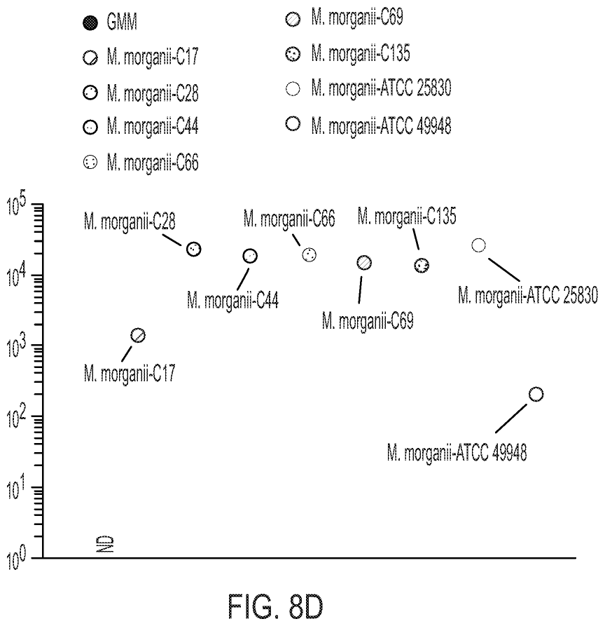

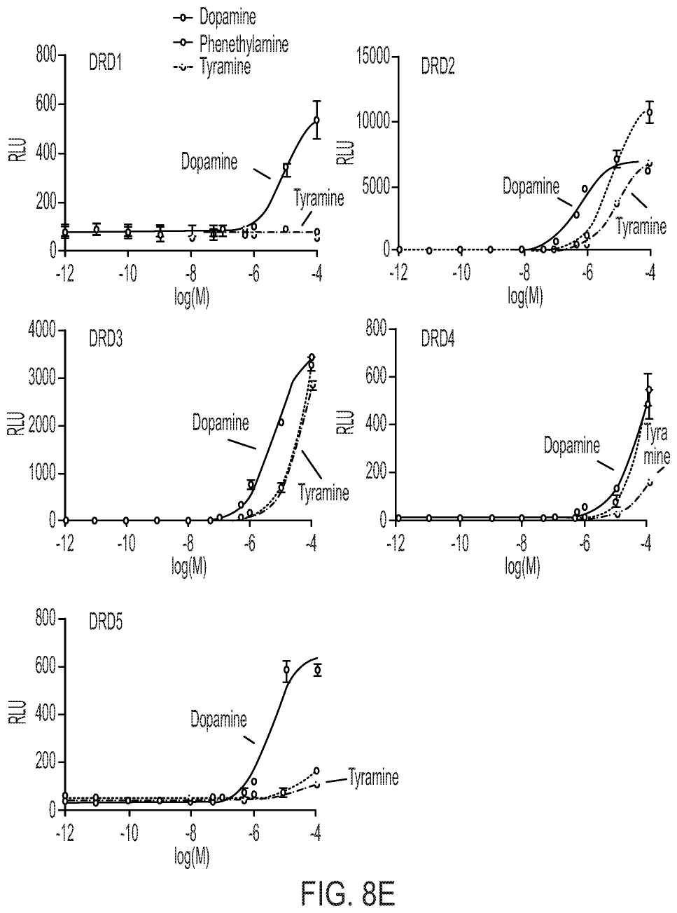

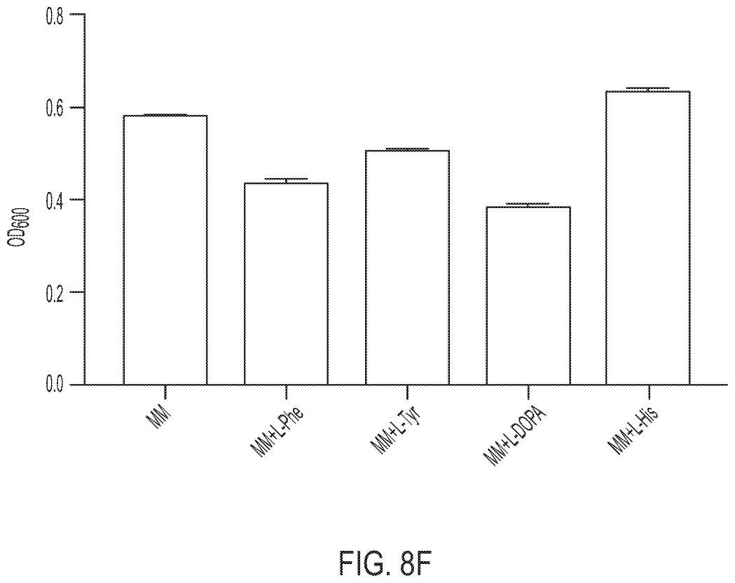

M. morganii was previously reported to produce dopamine as measured by high-performance liquid chromatography (HPLC) (Ozogul, 2004). However, it was observed that all M. morganii supernatants potently activated DRD2-4, but not DRD1 and DRD5, as shown in FIGS. 5A, 5B, 6A and 6B. In contrast, dopamine itself efficiently activated all five dopamine receptors as measured by PRESTO-Tango. See, FIG. 7A. Therefore, M. morganii might instead produce a metabolite that is structurally related to dopamine and can act as a selective ligand for DRD2-4 but not DRD1 or DRD5. See, FIG. 8A.

The inventors then examined the ability of all possible upstream and downstream metabolites in the mammalian dopamine pathway to activate DRD1-5 via PRESTO-Tango (FIGS. 8A, 8B). A variety of compounds in this pathway were found to activate various dopamine receptors; in particular, phenethylamine and tyramine showed identical activation patterns to M. morganii supernatant. See, FIGS. 8B and 8E. The concentrations of dopamine, phenethylamine, and tyramine in M. morganii supernatants were assayed. It was found that M. morganii produced only trace amounts of dopamine and no detectable tyramine. Instead M. morganii secreted significant quantities of the potent trace amine phenethylamine which, unlike dopamine and tyramine, can readily cross the blood-brain barrier. See, FIGS. 5D, 8C and 8D. (Oldendorf, 1971).

Previous reports have also suggested that M. morganii produces histamine (Ozogul, 2004). M. morganii strains indeed secreted significant amounts of histamine, as confirmed by ELISA (FIG. 5E). The histamine ELISA was conducted as follows. All strains were cultured in gut microbiota medium with or without 1% L-His for 24 hours and supernatants were collected via high-speed centrifugation. Cecal contents, colonic contents and fecal samples were collected and weighed; all samples were suspended in PBS at a ratio of 1:2 (w/v) and were homogenized by vortexing. Serum and brains were collected, weighed and suspended in PBS at a ratio of 1:2 (w/v). Brains were homogenized by passing them through a 21 G needle fifty times. All samples were centrifuged and supernatants were collected for histamine ELISA according to the manufacturer's protocol.

Similarly, two strains of L. reuteri and two strains from the Enterobacteriaceae family that activated histamine receptors also secreted histamine (FIG. 5E). Together, these data reveal that M. morganii secretes high levels of phenethylamine, which acts as a potent and selective agonist for a defined subset of the dopamine receptors, and that M. morganii and select strains of L. reuteri secrete histamine.

In mammals, phenethylamine, dopamine, and tyramine are produced via the decarboxylation of L-Phe, L-DOPA, and L-Tyr, respectively, by the aromatic L-amino acid decarboxylase (AADC; FIG. 8A) (Lovenberg, 1962). The inventors tested whether M. morganii would similarly process these three amino acids into their respective biogenic amines. A defined minimal medium (MM) lacking L-Phe, L-DOPA, L-Tyr, and L-His was used to culture M. morganii. Despite normal M. morganii growth, the inventors could not detect any production of phenethylamine, tyramine, dopamine, or histamine by liquid chromatography-mass spectrometry (LC-MS) (FIG. 5F).

However, supplementation with L-Phe or L-His led to the production of high levels of phenethylamine or histamine, and activation of DRD2-4 or HRH2-4, as shown in FIGS. 5F and 5G. In contrast, supplementation with L-DOPA or L-Tyr failed to lead to the production of dopamine or tyramine, or activation of DRDs by M. morganii supernatants despite similar bacterial growth in all conditions, as shown in FIG. 8F. This result suggests that, unlike mammalian AADC, M. morganii selectively converts L-Phe into phenethylamine and cannot efficiently convert L-DOPA or L-Tyr into dopamine or tyramine.

### Example 4. Microbiota-Derived Histamine Promotes Increased Colonic Motility

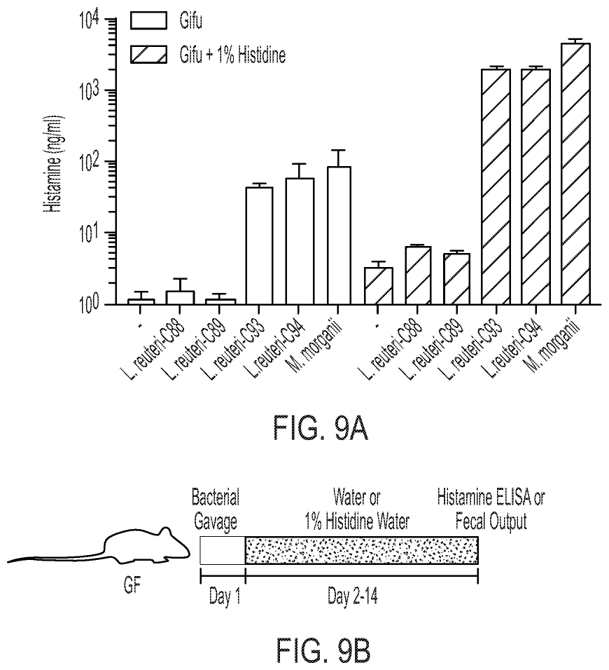

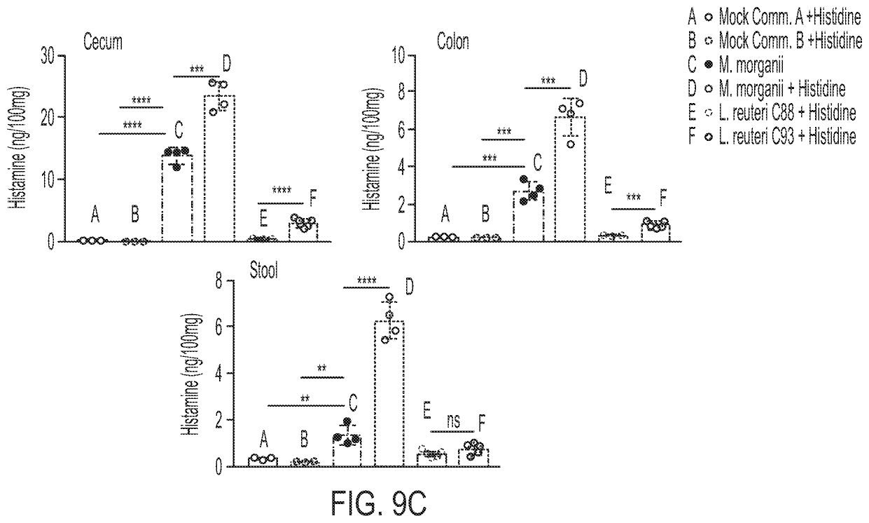

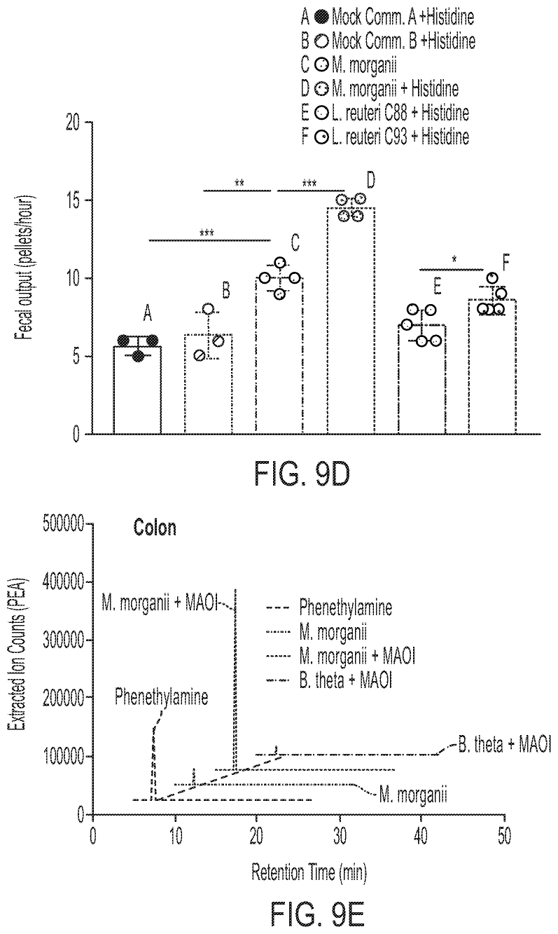

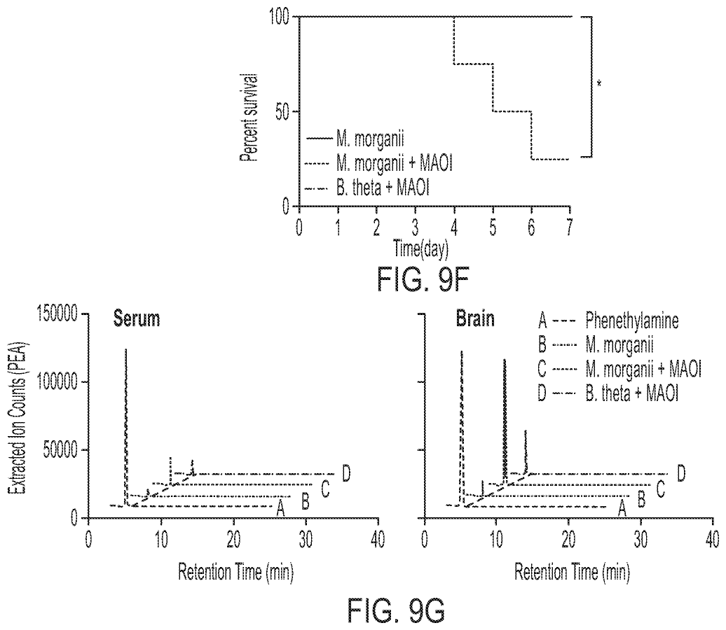

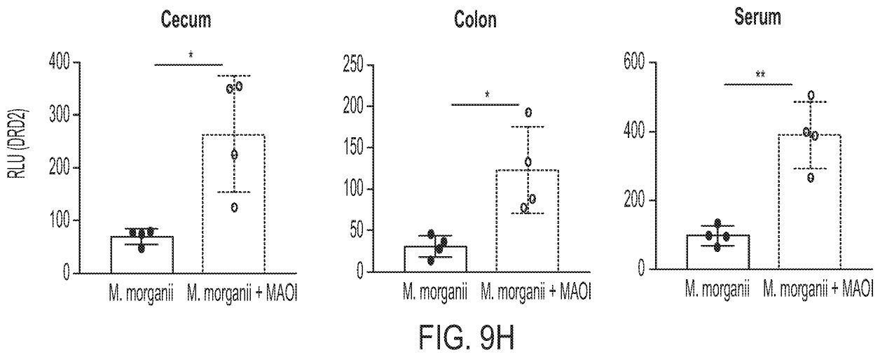

Histamine is generated via decarboxylation of L-His (Tannase, 1985). Eight strains of M. morganii and two strains of L. reuteri generated histamine in vitro. Supplementation with L-His significantly increased histamine production by these strains. In contrast, two distinct strains of L. reuteri failed to produce histamine regardless of supplementation with L-His, according to the results shown in FIG. 9A. When M. morganii was cultured in multiple media, either aerobically or anaerobically, M. morganii supernatants activated DRDs and HRHs regardless of culture conditions.

To test whether M. morganii and L. reuteri can also produce histamine in vivo, germ-free mice were colonized with two distinct mock communities containing (i) 9 or 10 diverse human gut microbes or (ii) M. morganii, with or without supplementation of 1% L-His in the drinking water ad libitum to approximate the effect of an L-His-rich diet, e.g., a meat-heavy diet. 6-12 weeks-old germ-free wild-type C57Bl/6 mice of both sexes were used in all experiments. The germ-free C57Bl/6 mice were colonized via oral gavage with 200 μl of individual bacterial cultures or mixed bacterial consortia. Mock communities A and B consisted of the following taxa: Community A: Streptococcus spp; C. perfringens; B. fragilis; Erisipelotrichaceae spp; C. aerofaciens; Bacteroides UC; B. producta; Allobaculum spp and Oscillospira spp; and Community B: Bacteroides spp; P. distasonis; Peptoniphilus spp; B. ovatus; Clostridiales UC/UC; Lachnospiraceae UC/UC; C. stercoris; B. uniformis and Parabacteroides spp. All gnotobiotic mice were maintained in Techniplast P Isocages and manipulated aseptically for the duration of the experiment.

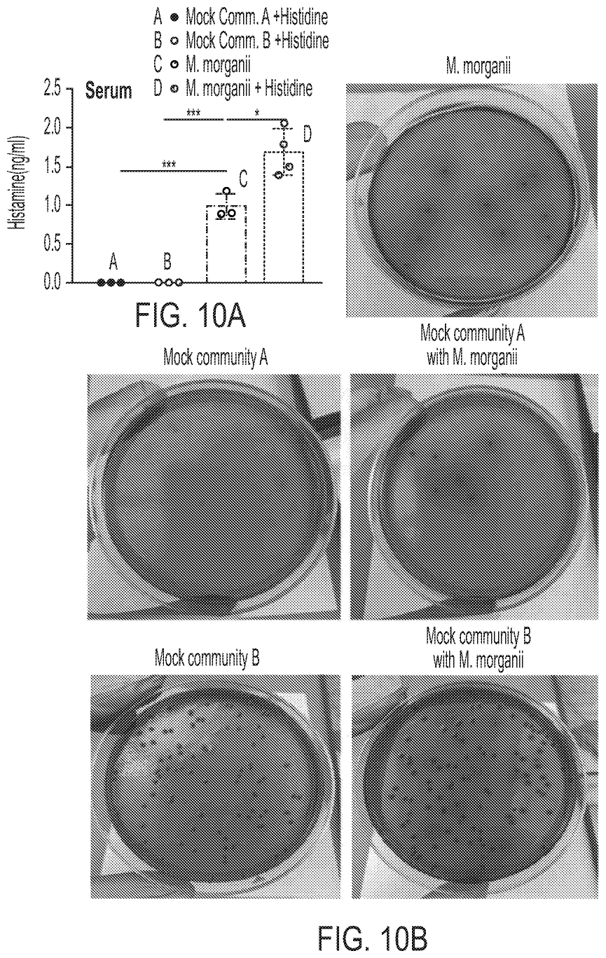

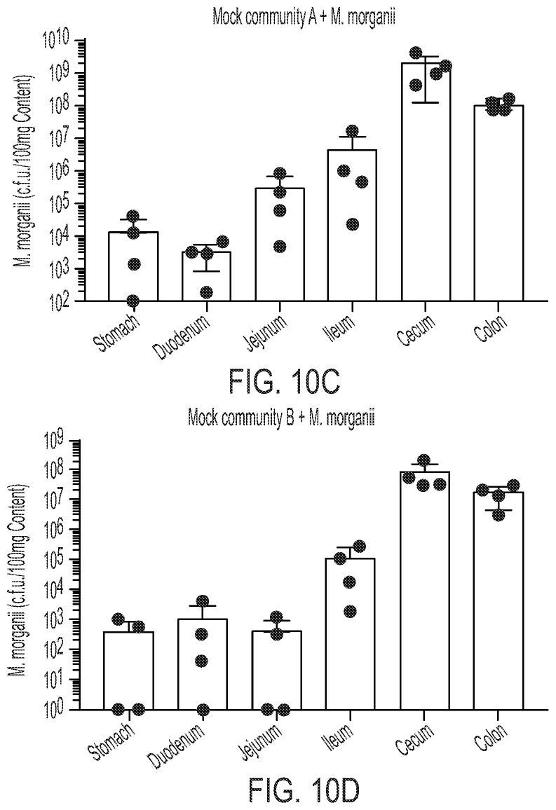

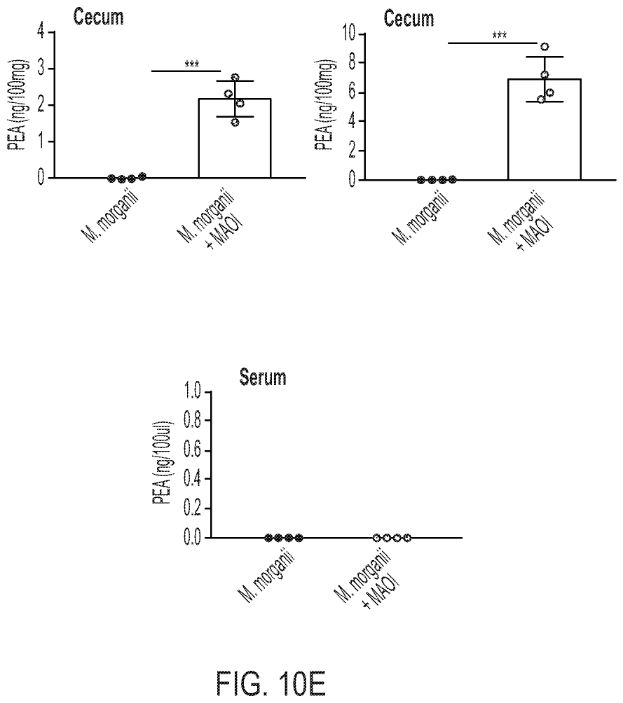

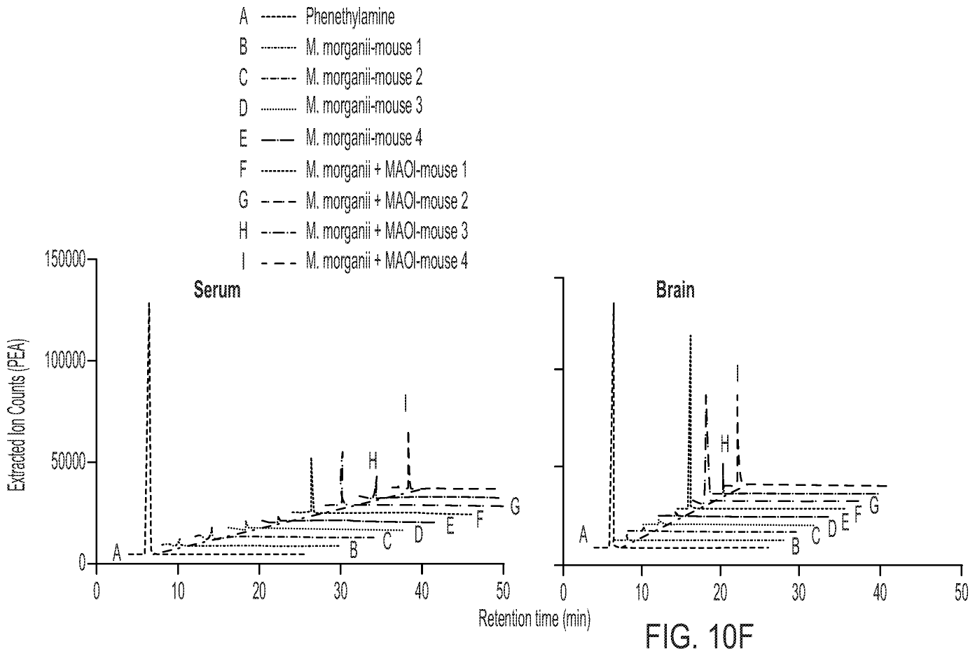

The data is shown in FIG. 9B. In addition, the inventors monocolonized mice with two strains of L. reuteri with divergent histamine production capabilities: L. reuteri C93, which produced significant histamine in vitro, and L. reuteri C88, which failed to produce histamine in vitro. Mice colonized with M. morganii or L. reuteri C93 exhibited high levels of intestinal histamine production, while mice colonized with the two mock communities or L. reuteri C88 showed nearly undetectable intestinal histamine, as shown in FIG. 9C. In addition, histamine production in M. morganii monocolonized mice was significantly increased upon supplementation with dietary L-His. See, FIG. 9C. As shown in FIG. 10A, increased histamine was detected in the serum of mice colonized with M. morganii, with or without feeding with additional L-His.

The location of M. morganii was next determined in vivo. Modified Niven's agar was used to enumerate M. morganii CFUs in gnotobiotic mice colonized with two mock communities of 9 or 10 diverse human gut microbes plus M. morganii (Mavromatis, 2002). The inventors found that M. morganii primarily inhabits the cecum and colon, and is nearly absent in the small intestine. See, FIG. 10B-10D. Previous studies in humans also indicated that M. morganii preferentially localizes in tissue- or mucus-associated niches in the colon (Eun et al., 2016).

Oral gavage with histamine has been reported to increase colon motility in rodents (Kim et al., 2011; Tyagi et al., 2009). The inventors thus hypothesized that gut microbe-derived histamine might also increase intestinal motility. The inventors assayed intestinal motility in mice colonized with two mock communities containing phylogenetically diverse human gut microbes or with M. morganii with or without administration of 1% L-His in the water. Intestinal motility was assayed by measuring fecal output as follows. Individual mice were housed in an empty container (¼ gallon) for 1 hour after which time the fecal pellets were counted and weighed. For mice fed with L-His, mice were given water containing 1% L-His ad libitum for 2 weeks before fecal output measurements.

Colonization with M. morganii led to a significant increase in fecal output, which was further increased upon supplementation with L-His. See, FIG. 9D. Similarly, mice colonized with L. reuteri C93 showed increased fecal output as compared to mice colonized with L. reuteri C88. See, FIG. 9D. These data indicate that microbiota-derived histamine can control intestinal motility and that dietary histidine can enhance these effects.

### Example 5. M. morganii can Trigger ‘Phenethylamine Poisoning’ when Combined with Monoamine Oxidase Inhibition

While abundant histamine production by M. morganii was detected both in vitro and in vivo, only low levels of phenethylamine were detected in the colons of M. morganii colonized mice (FIG. 9E). A possible explanation for this observation is that biogenic amines, such as dopamine, tyramine, and phenethylamine, are rapidly degraded in the intestine by mammalian monoamine oxidases (MAOs) (Glover, 1977). To reveal the potential production of phenethylamine in vivo, the inventors treated germ-free mice or mice monocolonized with M. morganii or a Bacteroides thetaiotaomicron (B. theta) strain that does not produce DRD agonists with the irreversible MAO inhibitor (MAOI) phenelzine.

Increased phenethylamine was observed in the colons of M. morganii colonized mice by triple quadrupole MS (FIG. 9E); in contrast, colonic phenethylamine was undetectable in germ-free mice and mice colonized with B. theta and treated with MAOI (FIG. 9E). Triple quadrupole (QQQ) MS was conducted as follows. Electron Spray Ionization-Triple Quadrupole-Tandem Mass Spectrometry ESI-QQQ-MS/MS was run using Multiple Reaction Monitoring (MRM) mode. An Agilent 6490 ESI-QQQ-MS/MS instrument with a Phenomenex Kinetex 1.7 μm C18 100 Å (100×2.10 mm) column was used for quantitation and calibration. Each standard (L-Phenylalanine, Phenethylamine, Histamine, Tyramine, Dopamine) was optimized using an Agilent Mass Hunter Optimizer. A calibration curve for each standard was established using various concentrations (0-25 μM range) in triplicate. The gradient constituted 10-100% acetonitrile in ddH₂O (with 0.1% Formic Acid), then a wash step with 100% acetonitrile. The triplicate data was then subjected to linear regression analysis to produce a linear calibration curve. Processing of the experimental samples involved lyophilization and extraction with 100% MeOH (20% volume of original culture volume) before injecting samples. Sample absorbance was subjected to linear calibration to calculate concentrations.

Unlike many other irreversible MAOIs, phenelzine is still used clinically for the treatment of major depressive disorder, as well as a variety of other psychological disorders including panic, social anxiety, and post-traumatic stress disorders (Fiedorowicz, 2004). The inventors found that mice colonized with M. morganii became lethargic within days after treatment with MAOI, and more than half of all mice colonized with M. morganii died before the seventh day of treatment. See, FIG. 9F. In contrast, mice monocolonized with B. theta and treated with MAOI appeared healthy.

Morbidity and mortality after MAOI treatment correlated with elevated levels of phenethylamine in the colon, serum and brains of M. morganii monocolonized mice treated with phenelzine, as measured by QQQ MS. The results are shown in FIGS. 9G, 10E and 10F. Finally, the inventors found that cecal and colonic contents and serum from MAOI-treated M. morganii colonized mice activated DRD2. See, FIG. 9H. These data show that M. morganii-derived phenethylamine can accumulate systemically and exert dramatic biological effects in mice treated with MAOIs.

### Example 6. A Unique Bacteroides Isolate Activates GPR56/AGRG1

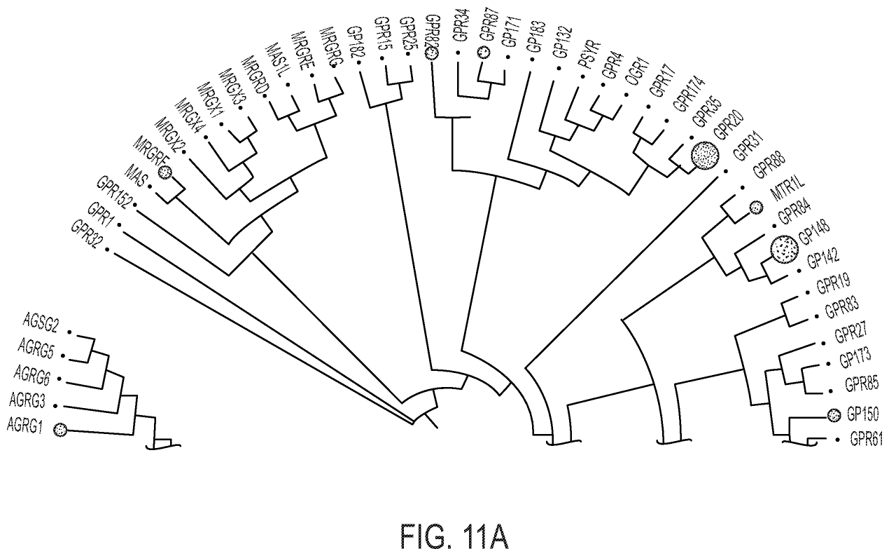

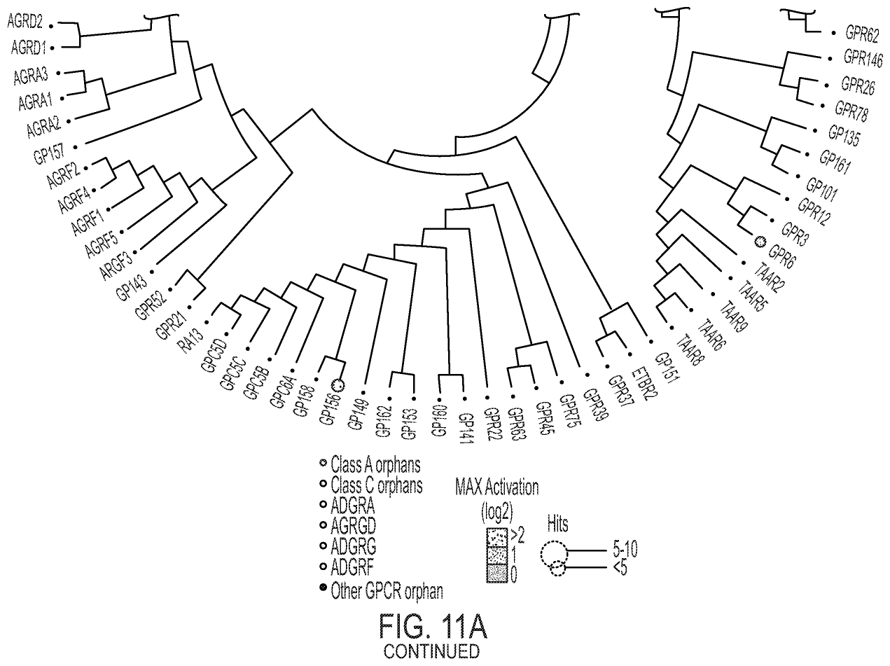

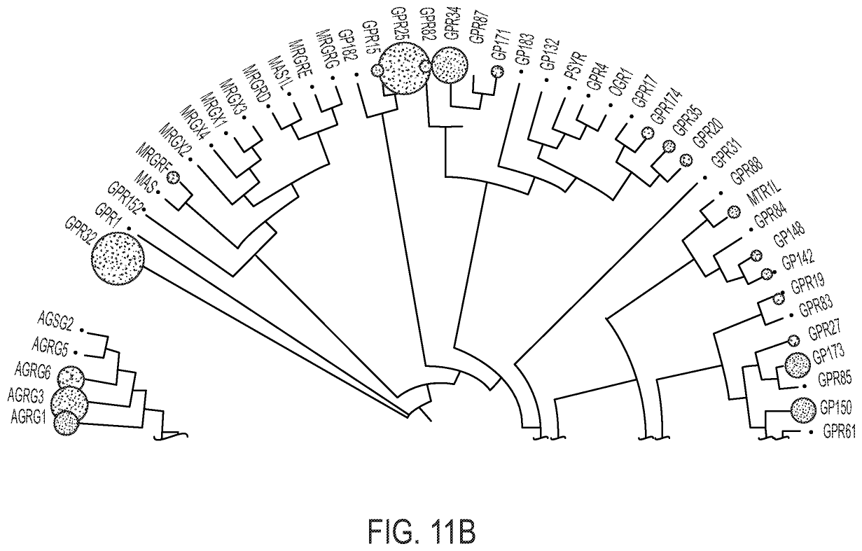

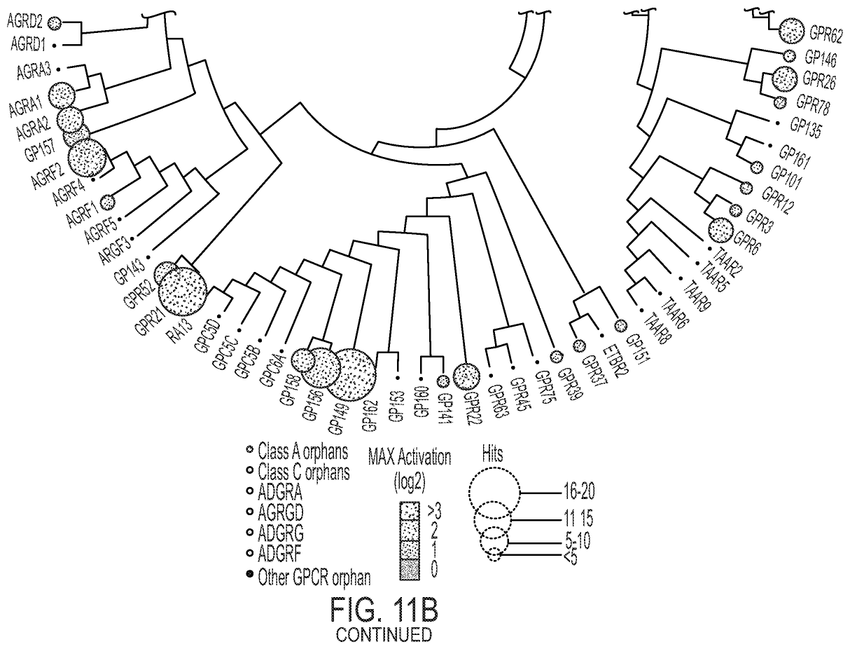

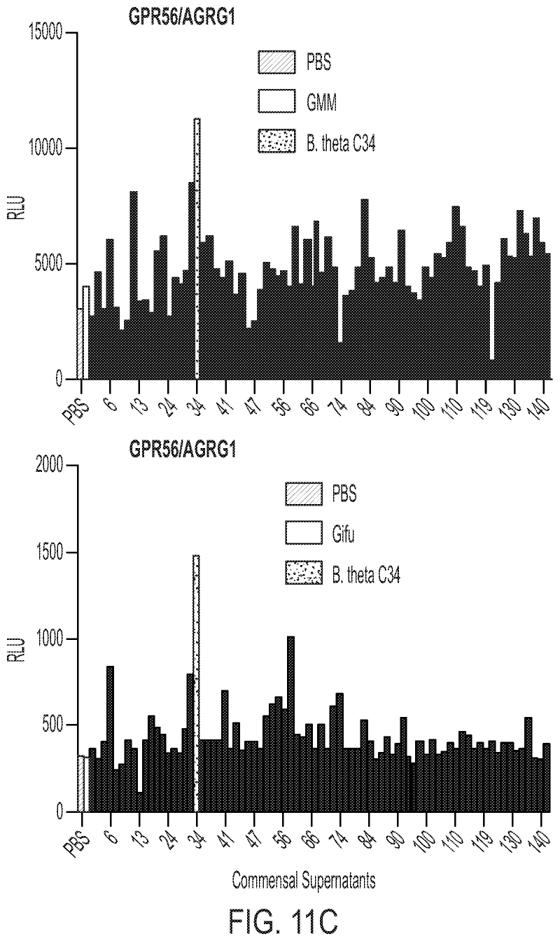

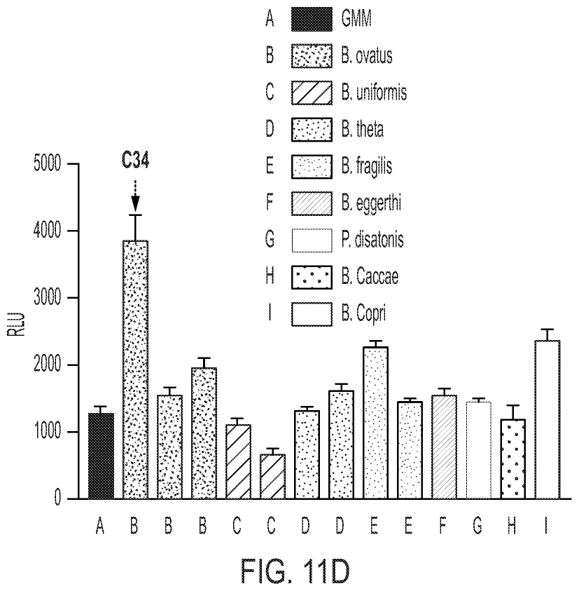

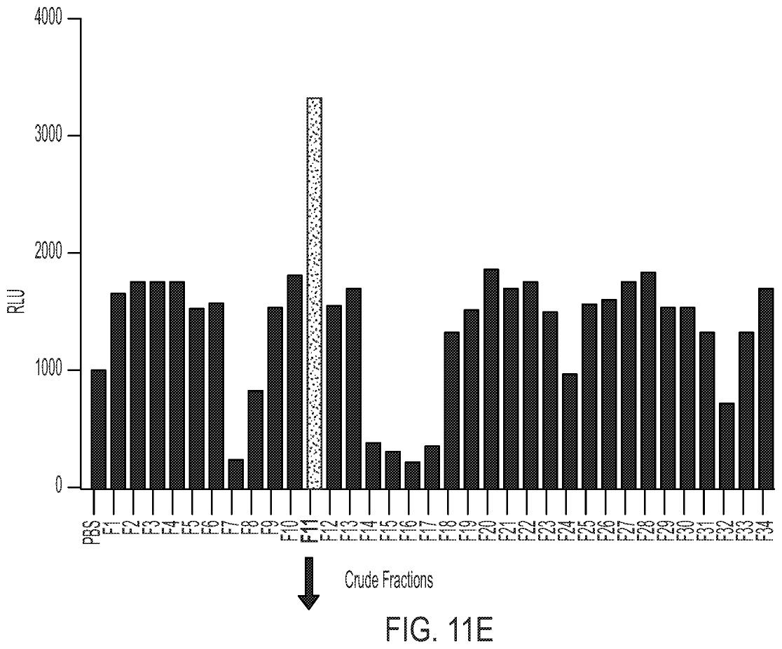

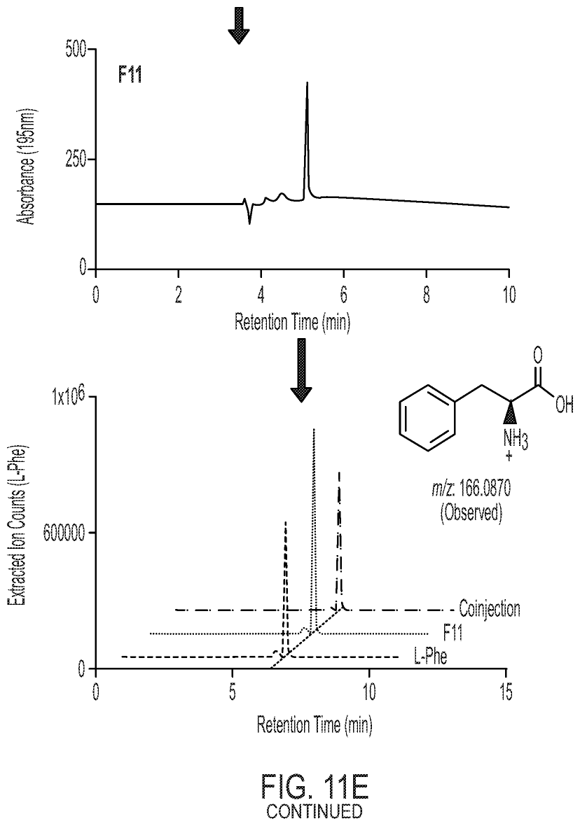

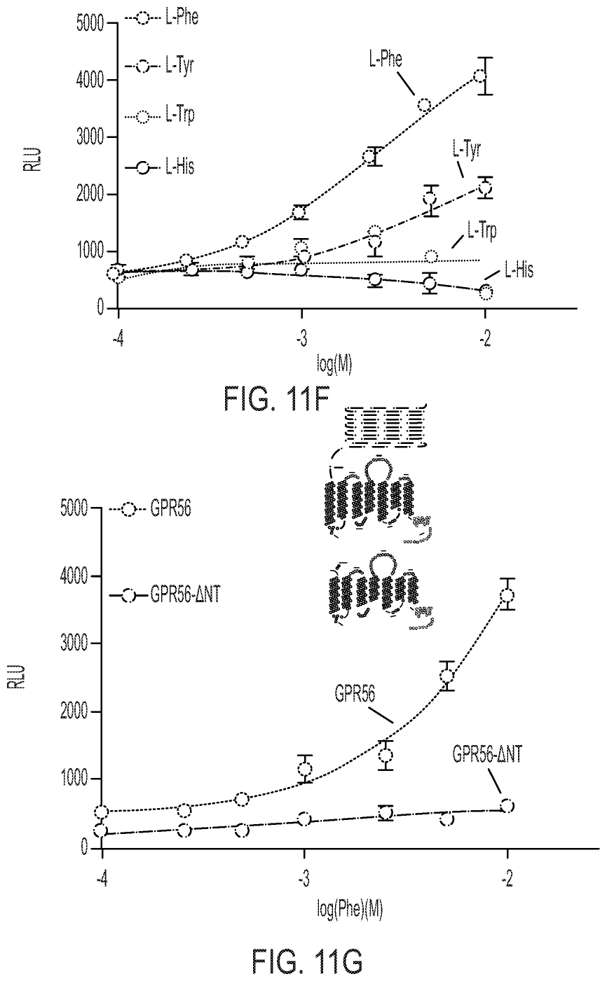

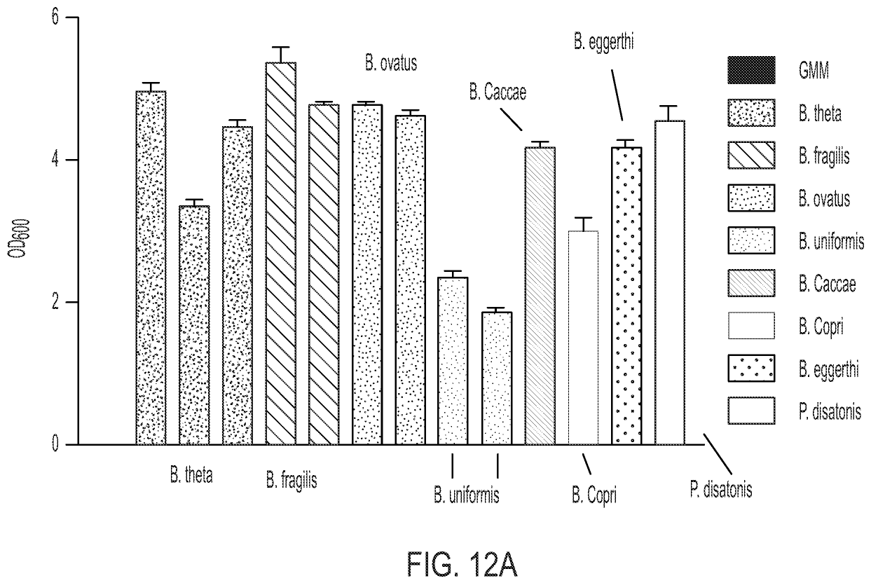

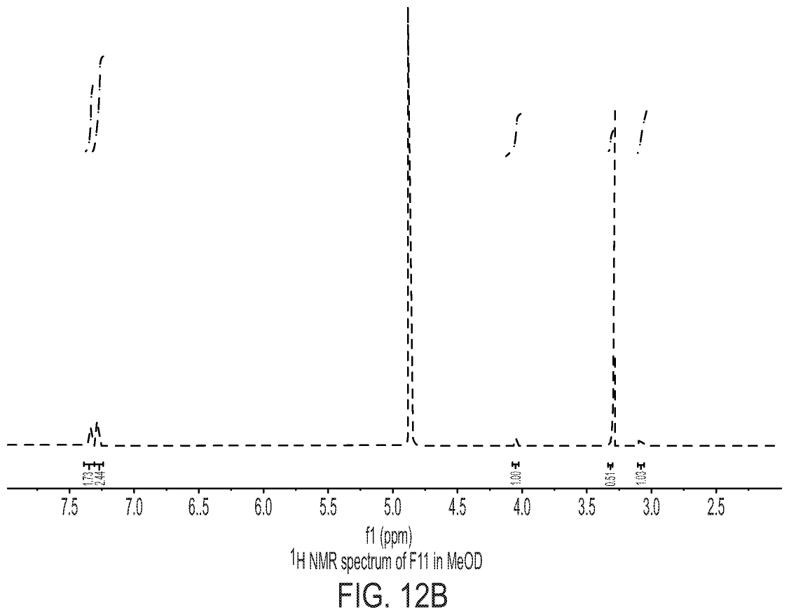

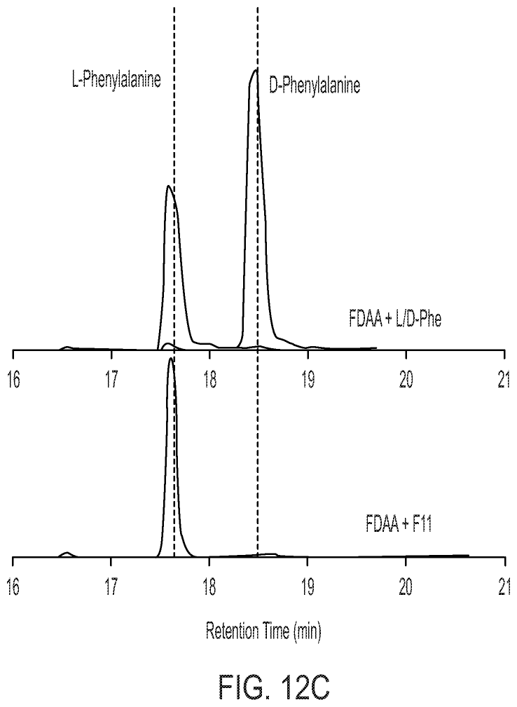

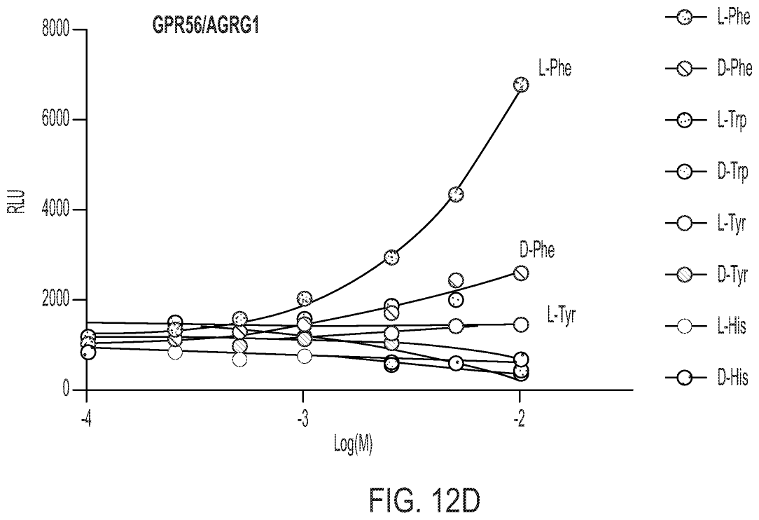

Specific bacterial supernatants were observed to activate select orphan GPCRs (FIG. 11A). To confirm, the inventors repeated our PRESTO-Tango screening procedure using a richer culture medium (Gifu) that supports more robust growth of most of the human gut microbes in our culture collection. This modified procedure significantly expanded the number of positive hits against orphan GPCRs: 17 orphan GPCRs showed greater than four-fold activation over media only controls in response to at least one bacterial supernatant, as shown in FIG. 11B. Metabolites from a strain assigned to the species B. theta (B. theta C34) activated GPR56/AGRG1 under both culture conditions (FIG. 11C). In contrast, other strains of B. theta, including commercially available strains of B. theta and multiple other Bacteroides species, failed to activate GPR56/AGRG1 (FIG. 11D) despite similar bacterial growth (FIG. 12A).

### Example 7. The Essential Amino Acid L-Phe Activates GPR56/AGRG1 and GPR97/AGRG3

Since there is no known endogenous small molecule ligand for GPR56/AGRG1 (Purcell, 2018), the inventors next attempted to identify the specific metabolite produced by B. theta C34 that activated GPR56/AGRG1. B. theta C34 supernatants were lyophilized, extracted with methanol and subjected to fractionation by reversed-phase HPLC. All resulting fractions were analyzed for activity via GPR56 PRESTO-Tango, with fraction 11 was identified as the active fraction. See, FIG. 11E.

Metabolomic assays were performed as follows. NMR spectra were taken using an Agilent 600 MHz NMR system with a cryoprobe. High-resolution MS and tandem MS (MS/MS) data were obtained using an Agilent iFunnel 6550 ESI-HRMS-QTOF (Electron Spray Ionization-High Resolution Mass Spectrometry-Quadrupole Time-of-Flight) instrument on Phenomenex Kinetex 5 μm C18 100 Å (4.6×250 mm) columns. The Agilent 1260 Infinity system with a Phenomenex Kinetex 5 μm C18 100 Å column (4.6×250 mm) or an Agilent Poroshell 120 EC-C18 2.7 μm (3.0×50 mm) column and a photodiode array (PDA) detector was used for routine sample analysis. An Agilent Prepstar HPLC system with an Agilent Polaris C18-A 5 μm (21.2×250 mm) columns were used for sample fractionation and purification.

High resolution mass spectrometry, NMR and coinjection analyses of F11 showed that the essential amino acid phenylalanine (Phe) is the primary constituent of F11 (FIGS. 11E and 12B). Finally, structural characterization using advanced Marfey's analysis confirmed that L-Phe is the likely bioactive ligand (FIG. 12C) (Bhushan and Bruckner, 2011).

The inventors next tested whether pure L-Phe or structurally related amino acids could activate GPR56/AGRG1 using GPR56-Tango. B. thetaiotaomicron strain C11 was grown in 10 mL of gut microbiota medium under anaerobic conditions at 37° C. for 24 hr. Supernatant was harvested, lyophilized and extracted with 2 mL methanol. The crude extract was then dried and fractionated using a preparative C18 HPLC system. The gradient used was 10-50% acetonitrile in water (with 0.01% trifluoroacetic acid) for 30 min, then 100% for 5 min. The fractions, which were collected every minute, were dried, resuspended in PBS buffer, and tested for bioactivity using PRESTO-Tango. The active fraction was characterized using ESI-HRMS-QTOF and NMR analyses. Stereochemistry was confirmed by advanced Marfey's analysis (FIG. 12D) (Bhushan and Bruckner, 2011).

L-Phe and, to a lesser extent, L-Tyr stereoselectively activated GPR56/AGRG1, while L-Trp and L-His, D-Phe, D-Trp, D-His and D-Tyr showed no activity. See, FIGS. 11F and 12D.

GPR56/AGRG1 is a member of the adhesion GPCR family. Adhesion GPCRs characteristically possess large extracellular domains that mediate interactions with a variety of protein ligands, such as components of the extracellular matrix (Purcell, 2018). The inventors assayed whether the extracellular domain of GPR56/AGRG1 was also required for activation by the small molecule L-Phe by constructing a truncation mutant of GPR56/AGRG1. Although this mutant is expressed normally (Kishore et al., 2016), it failed to respond to L-Phe. See, FIG. 11G. The data suggest that the extracellular domain of GPR56/AGRG1 is critical for its activation by L-Phe, a unique strain of B. theta secretes high levels of L-Phe, and L-Phe is a novel agonist of the adhesion GPCR GPR56/AGRG1.

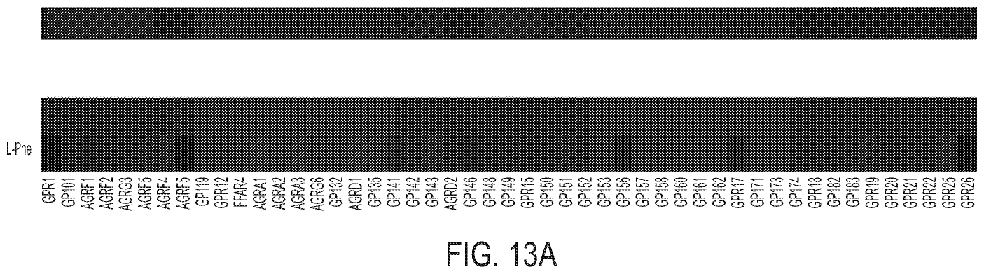

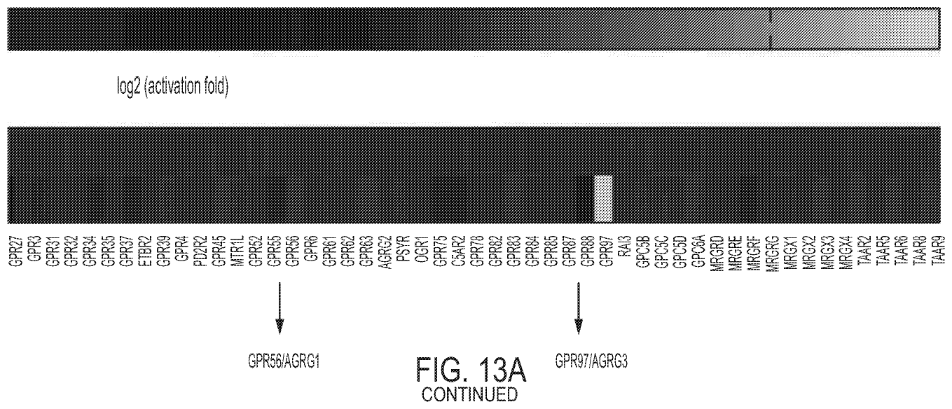

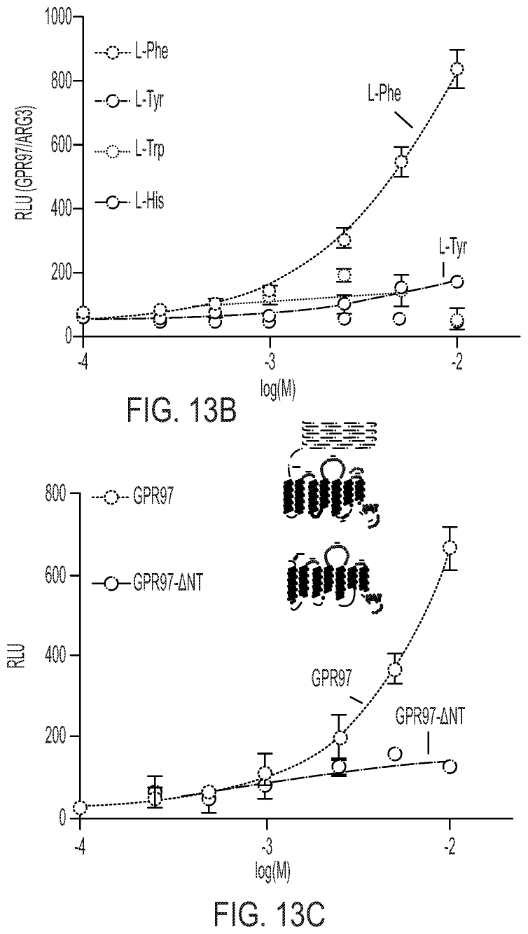

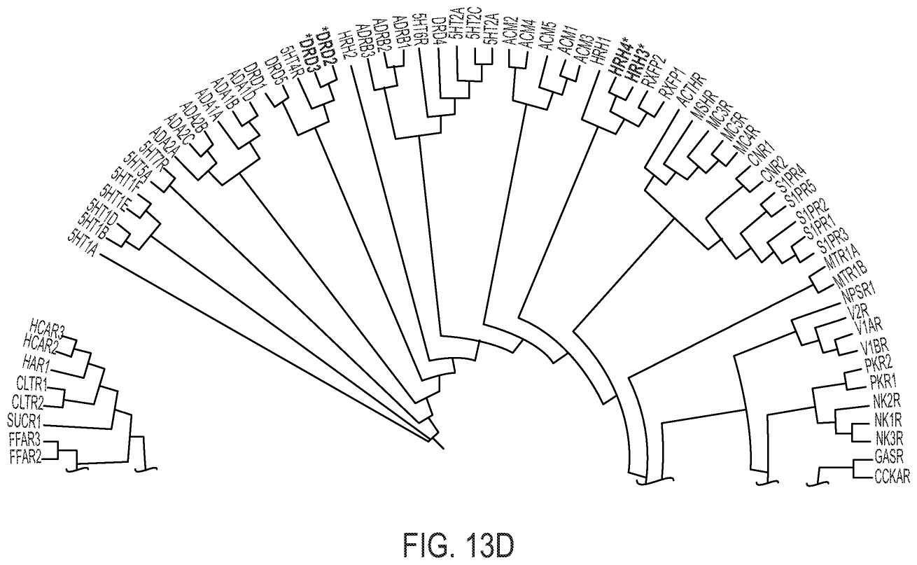

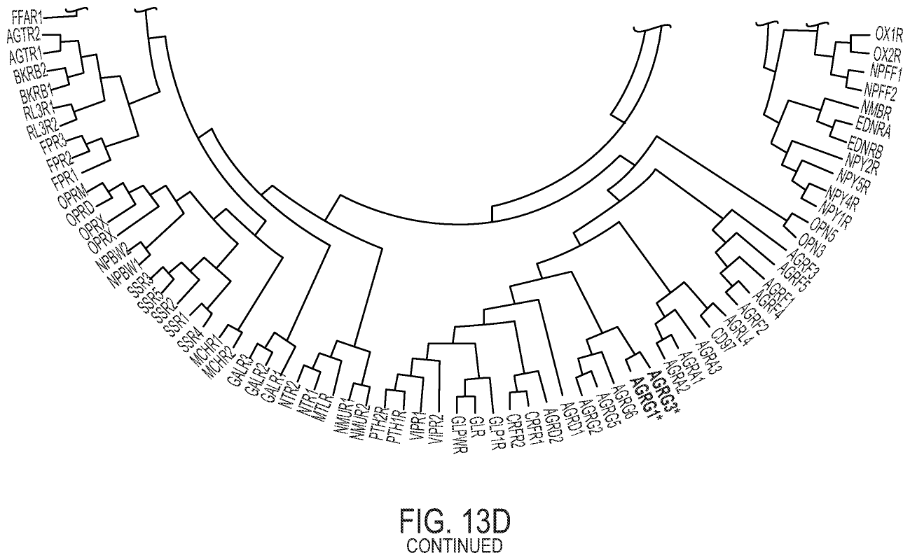

The inventors next examined whether other orphan GPCRs might also respond to L-Phe. PRESTO-Tango screening was performed on all adhesion and orphan GPCRs stimulated with L-Phe. It was found that GPR97/AGRG3 was also activated by this compound, as shown in FIG. 13A. Upon further analysis, GPR97/AGRG3 showed greater selectivity but lower sensitivity towards L-Phe than GPR56/AGRG1—L-Phe, but not L-Tyr, L-Trp, or L-His, activated GPR97/AGRG3 (FIG. 13B). The result may explain why no significant activation of GPR97/AGRG3 by B. theta C34 supernatant was initially observed. Like GPR56/AGRG1, the extracellular domain of GPR97/AGRG3 was required for its ability to respond to L-Phe (FIG. 13C). Notably, both GPR56/AGRG1 and GPR97/AGRG3 belong to the G family of adhesion GPCRs and are closely related evolutionarily, as shown in FIG. 13D, which may explain their shared ability to detect the essential amino acid L-Phe.

### Example 8. Bacterial Metabolic Exchange can Contribute to In Vivo Production of Phenethylamine

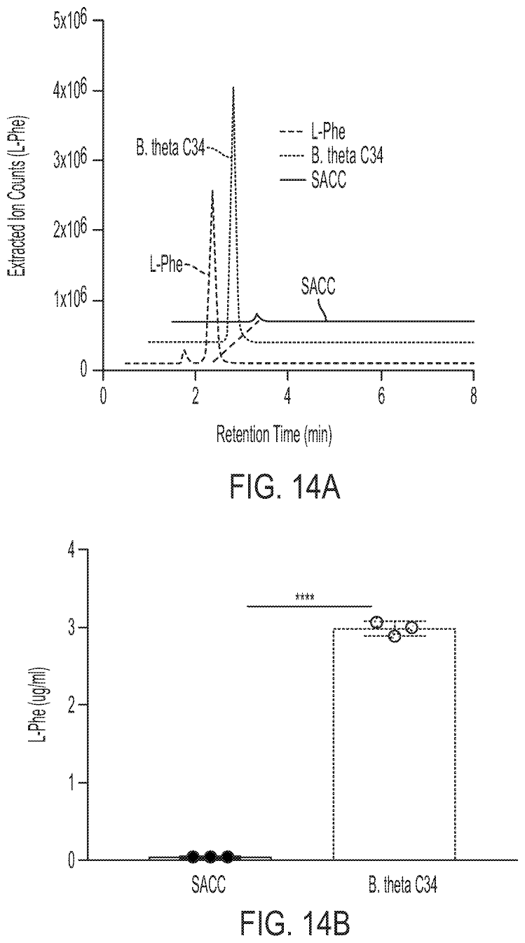

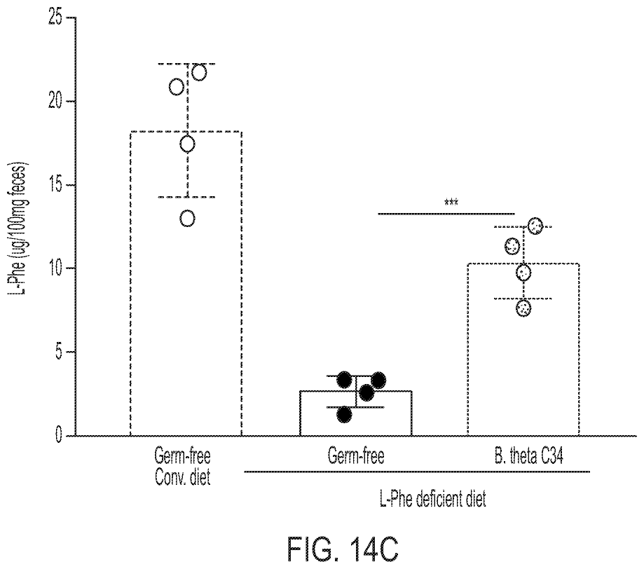

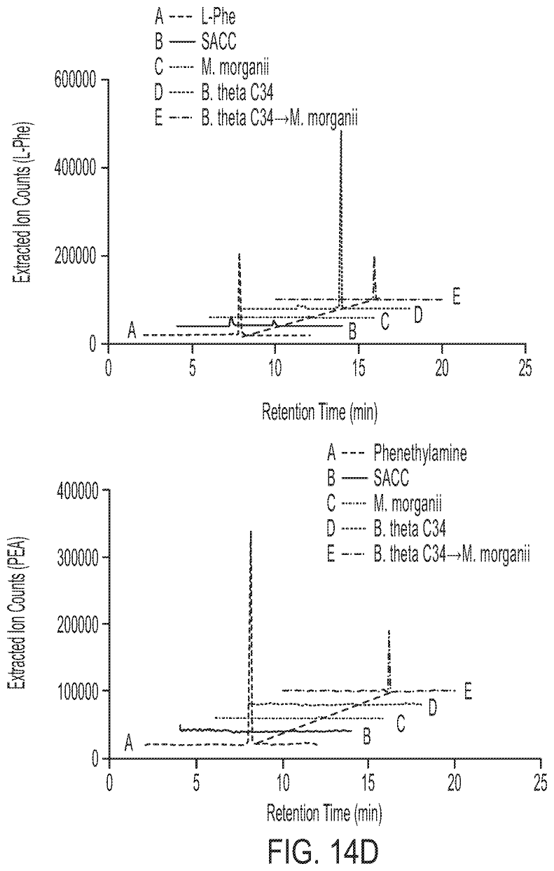

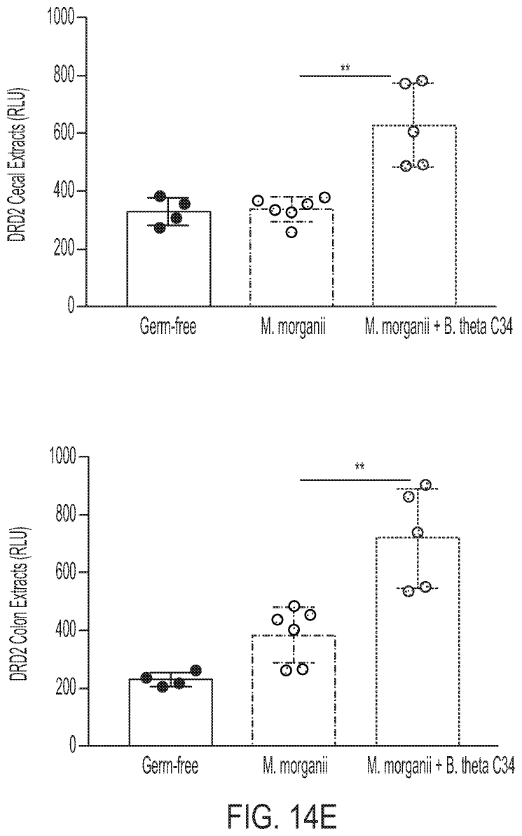

The above reductionist studies revealed that B. theta C34 produces large amounts of L-Phe while M. morganii can process L-Phe into the trace amine phenethylamine. The inventors then performed an assay to address whether these two bacteria might participate in an active metabolic exchange in vivo. The first step in investigating this hypothesis was to determine whether B. theta C34 can directly synthesize L-Phe. Using a defined minimal medium lacking L-Phe (SACC), the inventors observed that B. theta C34 could directly synthesize significant amounts of L-Phe in vitro (FIG. 14A, 14B). The inventors thus monocolonized mice fed an L-Phe deficient diet with B. theta C34 and evaluated the in vivo production of L-Phe via QQQ MS. GF mice fed with an L-Phe deficient diet exhibited reduced concentrations of L-Phe in the feces as compared to GF mice fed a conventional diet (FIG. 14C). In contrast, mice colonized with B. theta C34 and fed with an L-Phe-deficient diet exhibited significantly increased levels of L-Phe as compared to GF mice fed with an L-Phe-deficient diet.

The inventors next examined whether M. morganii would directly process B. theta C34-derived L-Phe into phenethylamine. B. theta C34 was cultured in SACC medium and then transferred B. theta supernatant to a culture of M. morganii. B. theta C34-derived L-Phe was efficiently converted into phenethylamine by M. morganii, as shown in FIG. 14D. To test whether metabolic exchange between C34 and M. morganii could contribute to in vivo production of phenethylamine, GF mice were colonized with either (i) M. morganii alone or (ii) both B. theta C34 and M. morganii. The mice were fed with a simplified diet lacking L-Phe.

These mice were then treated with phenelzine to reveal the potential production of phenethylamine. Mice colonized with M. morganii alone and fed an L-Phe deficient diet remained healthy and produced minimal phenethylamine (as measured by DRD2 activation by cecal and colonic extracts) despite MAOI treatment (FIG. 14E). In contrast, mice that were bi-colonized with C34 and M. morganii, fed an L-Phe deficient diet, and treated with MAOI became lethargic by day 4 and produced significant levels of phenethylamine as measured by DRD2 activation by cecal and colonic extracts (FIG. 14E). These results demonstrate that C34 and M. morganii can participate in an active metabolic exchange in vivo and that this exchange can contribute to the production of a bioactive trace amine that has potent effects on host physiology.

### Example 9. New Ultra-Highthroughput Combinatorial GPCR Screening Technology PRESTO-Salsa Enables Simultaneous Screening of Hundreds of GPCRs in a Single Well of a 96 Well Plate

Salsa reporter cells (a stable cell line expressing a fusion protein of B-arrestin and TEV protease) were seeded into poly-D-lysine pretreated 96-well plate with 1004, DMEM+10% FBS+1% Pen/Strep. When cell density reached 90%, the cells were transfected with 100 ng of plasmid encoding each GPCR and 100 ng of a unique Salsa reporter plasmid in each well (1 well per GPCR and 314 wells total). Six hours after transfection, the cell medium was discarded followed by addition of 50 μl trypsin and 10 min incubation at 37 degrees Celsius. After digestion, an additional 504, of cell medium was added, and cells were pipetted 15 times to separate cell clusters into single cells. All transfected cells were then pooled into one 15 mL tube to generate a mixed cell library and centrifuged at 3,000 rpm for 5 min.

The supernatant was discarded, and cells were resuspended in the same volume of fresh cell medium. The cell pellet was pipetted 15 times to resuspend the cells, and then cells were reseeded at 100 μL/well into poly-D-lysine pretreated 96-well plates. Twelve hours after reseeding, the cell medium was discarded and replaced with 1004, of fresh DMEM+1% Pen/Strep, followed by stimulation with serial dilutions of the indicated GPCR ligands. Nine hours after ligand stimulation, mRNA was extracted from each well and used to prepare a amplicon DNA library for next-generation sequencing. Barcode reads after ligand stimulation divided by barcode reads without ligand stimulation were calculated to produce the fold activation for each GPCR and for each sample.

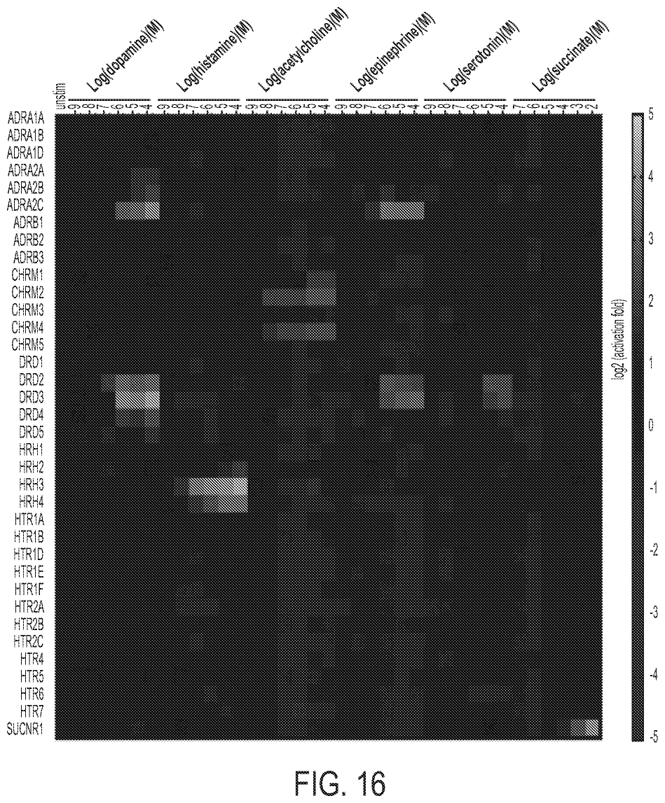

The data is shown in FIG. 16.

### REFERENCES

Adibi, S. M., D W. (1973). Protein Digestion in Human Intestine as Reflected in Luminal, Mucosal, and Plasma Amino Acid Concentrations after Meals. The Journal of Clinical Investigation 52, 1586-1594.
Albuquerque, E. X., Pereira, E. F., Alkondon, M., and Rogers, S. W. (2009). Mammalian nicotinic acetylcholine receptors: from structure to function. Physiol Rev 89, 73-120.
Amisten, S., Salehi, A., Rorsman, P., Jones, P. M., and Persaud, S. J. (2013). An atlas and functional analysis of G-protein coupled receptors in human islets of Langerhans. Pharmacol Ther 139, 359-391.
Barcik, W., Pugin, B., Westermann, P., Perez, N. R., Ferstl, R., Wawrzyniak, M., Smolinska, S., Jutel, M., Hessel, E. M., Michalovich, D., et al. (2016). Histamine-secreting microbes are increased in the gut of adult asthma patients. J Allergy Clin Immunol 138, 1491-1494 e1497.
Barnea, G., Strapps, W., Herrada, G., Berman, Y., Ong, J., Kloss, B., Axel, R., and Lee, K. J. (2008). The genetic design of signaling cascades to record receptor activation. Proc Natl Acad Sci USA 105, 64-69.
Beaulieu, J. M., and Gainetdinov, R. R. (2011). The physiology, signaling, and pharmacology of dopamine receptors. Pharmacol Rev 63, 182-217.
Bhushan, R., and Bruckner, H. (2011). Use of Marfey's reagent and analogs for chiral amino acid analysis: assessment and applications to natural products and biological systems. J Chromatogr B Analyt Technol Biomed Life Sci 879, 3148-3161.
Borowsky, B., Adham, N., Jones, K. A., Raddatz, R., Artymyshyn, R., Ogozalek, K. L., Durkin, M. M., Lakhlani, P. P., Bonini, J. A., Pathirana, S., et al. (2001). Trace amines: identification of a family of mammalian G protein-coupled receptors. Proc Natl Acad Sci USA 98, 8966-8971.
Bunzow, J. S., M S; Arttamangkul, S; Harrison, L M; Zhang, G; Quigley, D I; Darland, T; Suchland, K L; Pasumanmula, S; Kennedy, J L; Olson, S B; Magenis, R E; Amara, S G; Grandy, D K. (2001). Amphetamine, 3,4-Methylenedioxymethamphetamine, Lysergic Acid Diethylamide, and Metabolites of the Catecholamine Neurotransmitters Are Agonists of a Rat Trace Amine Receptor. Molecular Pharmacology 60, 1181-1188.
Cohen, L. J., Esterhazy, D., Kim, S. H., Lemetre, C., Aguilar, R. R., Gordon, E. A., Pickard, A. J., Cross, J. R., Emiliano, A. B., Han, S. M., et al. (2017). Commensal bacteria make GPCR ligands that mimic human signalling molecules. Nature 549, 48-53.
Dodd, D., Spitzer, M. H., Van Treuren, W., Merrill, B. D., Hryckowian, A. J., Higginbottom, S. K., Le, A., Cowan, T. M., Nolan, G. P., Fischbach, M. A., et al. (2017). A gut bacterial pathway metabolizes aromatic amino acids into nine circulating metabolites. Nature 551, 648-652.
Donia, M. S., and Fischbach, M. A. (2015). HUMAN MICROBIOTA. Small molecules from the human microbiota. Science 349, 1254766.
Duner, P., Al-Amily, I. M., Soni, A., Asplund, O., Safi, F., Storm, P., Groop, L., Amisten, S., and Salehi, A. (2016). Adhesion G Protein-Coupled Receptor G1 (ADGRG1/GPR56) and Pancreatic beta-Cell Function. J Clin Endocrinol Metab 101, 4637-4645.
Eun, C. S., Kwak, M. J., Han, D. S., Lee, A. R., Park, D. I., Yang, S. K., Kim, Y. S., and Kim, J. F. (2016). Does the intestinal microbial community of Korean Crohn's disease patients differ from that of western patients? BMC Gastroenterol 16, 28.
Fiedorowicz, J. S., K L. (2004). The Role of Monoamine Oxidase Inhibitors in Current Psychiatric Practice. J Psychiatr Pract 10, 239-248.
Fischbach, M. A. (2018). Microbiome: Focus on Causation and Mechanism. Cell 174, 785-790.
Giera, S., Luo, R., Ying, Y., Ackerman, S. D., Jeong, S. J., Stoveken, H. M., Folts, C. J., Welsh, C. A., Tall, G. G., Stevens, B., et al. (2018). Microglial transglutaminase-2 drives myelination and myelin repair via GPR56/ADGRG1 in oligodendrocyte precursor cells. Elife 7.
Glover, V. S., M; Owen, F; Riley, G J. (1977). Dopamine is a monoamine oxidase B substrate in man. Nature 265, 80-81.
Goodman, A. L., Kallstrom, G., Faith, J. J., Reyes, A., Moore, A., Dantas, G., and Gordon, J. I. (2011). Extensive personal human gut microbiota culture collections characterized and manipulated in gnotobiotic mice. Proc Natl Acad Sci USA 108, 6252-6257.
Guo, C. J., Chang, F. Y., Wyche, T. P., Backus, K. M., Acker, T. M., Funabashi, M., Taketani, M., Donia, M. S., Nayfach, S., Pollard, K. S., et al. (2017). Discovery of Reactive Microbiota-Derived Metabolites that Inhibit Host Proteases. Cell 168, 517-526 e518.
Haiser, H. J., Gootenberg, D. B., Chatman, K., Sirasani, G., Balskus, E. P., and Turnbaugh, P. J. (2013). Predicting and manipulating cardiac drug inactivation by the human gut bacterium Eggerthella lenta. Science 341, 295-298.
Haitina, T., Olsson, F., Stephansson, O., Alsio, J., Roman, E., Ebendal, T., Schioth, H. B., and Fredriksson, R. (2008). Expression profile of the entire family of Adhesion G protein-coupled receptors in mouse and rat. BMC Neurosci 9, 43.
Husted, A. S., Trauelsen, M., Rudenko, O., Hjorth, S. A., and Schwartz, T. W. (2017). GPCR-Mediated Signaling of Metabolites. Cell Metab 25, 777-796.
Irsfeld, M., Spadafore, M., and Pruss, B. M. (2013). beta-phenylethylamine, a small molecule with a large impact. Webmedcentral 4.
Kim, H., Dwyer, L., Song, J. H., Martin-Cano, F. E., Bahney, J., Peri, L., Britton, F. C., Sanders, K. M., and Koh, S. D. (2011). Identification of histamine receptors and effects of histamine on murine and simian colonic excitability. Neurogastroenterol Motil 23, 949-e409.
Kishore, A., Purcell, R. H., Nassiri-Toosi, Z., and Hall, R. A. (2016). Stalk-dependent and Stalk-independent Signaling by the Adhesion G Protein-coupled Receptors GPR56 (ADGRG1) and BAH (ADGRB1). J Biol Chem 291, 3385-3394.
Kroeze, W. K., Sassano, M. F., Huang, X. P., Lansu, K., McCorvy, J. D., Giguere, P. M., Sciaky, N., and Roth, B. L. (2015). PRESTO-Tango as an open-source resource for interrogation of the druggable human GPCRome. Nat Struct Mol Biol 22, 362-369.
Larsbrink, J., Rogers, T. E., Hemsworth, G. R., McKee, L. S., Tauzin, A. S., Spadiut, O., Klinter, S., Pudlo, N. A., Urs, K., Koropatkin, N. M., et al. (2014). A discrete genetic locus confers xyloglucan metabolism in select human gut Bacteroidetes. Nature 506, 498-502.
Lei, W., Ren, W., Ohmoto, M., Urban, J. F., Jr., Matsumoto, I., Margolskee, R. F., and Jiang, P. (2018). Activation of intestinal tuft cell-expressed Sucnr1 triggers type 2 immunity in the mouse small intestine. Proc Natl Acad Sci USA 115, 5552-5557.
Lovenberg, W. W., H & Udenfriend, S. (1962). Aromatic amino acid decarboxylase. The Journal of Biological Chemistry 237, 89-93.
Mavromatis, P. Q., P C. (2002). ModifiIcation of Niven's Medium for the Enumeration of Histamine-Forming Bacteria and Discussion of the Parameters Associated with Its Use. Journal of Food Protection 65, 546-551.
Milshteyn, A., Colosimo, D. A., and Brady, S. F. (2018). Accessing Bioactive Natural Products from the Human Microbiome. Cell Host Microbe 23, 725-736.
Nadjsombati, M. S., McGinty, J. W., Lyons-Cohen, M. R., Jaffe, J. B., DiPeso, L., Schneider, C., Miller, C. N., Pollack, J. L., Nagana Gowda, G. A., Fontana, M. F., et al. (2018). Detection of Succinate by Intestinal Tuft Cells Triggers a Type 2 Innate Immune Circuit. Immunity 49, 33-41 e37.
Nicholson, J. K., Holmes, E., Kinross, J., Burcelin, R., Gibson, G., Jia, W., and Pettersson, S. (2012). Host-gut microbiota metabolic interactions. Science 336, 1262-1267.
Oldendorf, W. (1971). Brain uptake of radiolabeled amino acids, amines, and hexoses after arterial injection. American Journal of Physiology 221, 1629-1639.
Özoğul, F. (2004). Production of biogenic amines by Morganella morganii, Klebsiella pneumoniae and Hafnia alvei using a rapid HPLC method. European Food Research and Technology 219, 465-469.
Palm, N. W., de Zoete, M. R., Cullen, T. W., Barry, N. A., Stefanowski, J., Hao, L., Degnan, P. H., Hu, J., Peter, I., Zhang, W., et al. (2014). Immunoglobulin A coating identifies colitogenic bacteria in inflammatory bowel disease. Cell 158, 1000-1010.
Pedersen, H. K., Gudmundsdottir, V., Nielsen, H. B., Hyotylainen, T., Nielsen, T., Jensen, B. A., Forslund, K., Hildebrand, F., Prifti, E., Falony, G., et al. (2016). Human gut microbes impact host serum metabolome and insulin sensitivity. Nature 535, 376-381.
Perry, R. J., Peng, L., Barry, N. A., Cline, G. W., Zhang, D., Cardone, R. L., Petersen, K. F., Kibbey, R. G., Goodman, A. L., and Shulman, G. I. (2016). Acetate mediates a microbiome-brain-beta-cell axis to promote metabolic syndrome. Nature 534, 213-217.
Pezeshki, A., Zapata, R. C., Singh, A., Yee, N. J., and Chelikani, P. K. (2016). Low protein diets produce divergent effects on energy balance. Scientific Reports 6.
Purcell, R. H., R A. (2018). Adhesion G Protein-Coupled Receptors as Drug Targets. Annu Rev Pharmacol Toxicol 58, 429-449.
Ramachandraih, C. S., N; Bar, K J; Baker, G; Yeragani, V K. (2011). Antidepressants: From MAOIs to SSRIs and more. Indian J Psychiatry 53, 180-182.
Riederer, P., and Laux, G. (2011). MAO-inhibitors in Parkinson's Disease. Exp Neurobiol 20, 1-17.
Schneider, C., O'Leary, C. E., von Moltke, J., Liang, H. E., Ang, Q. Y., Turnbaugh, P. J., Radhakrishnan, S., Pellizzon, M., Ma, A., and Locksley, R. M. (2018). A Metabolite-Triggered Tuft Cell-ILC2 Circuit Drives Small Intestinal Remodeling. Cell 174, 271-284 e214.
Smith, P. H., M R; Panikov, N; Michaud, M; Gallini, C A; Bohlooly-Y, M; Glickman, J N; Garret, W S. (2013). The Microbial Metabolites, Short-Chain Fatty Acids, Regulate Colonic Treg Cell Homeostasis. Science 341, 569-573.
Sotnikova, T. D., Budygin, E. A., Jones, S. R., Dykstra, L. A., Caron, M. G., and Gainetdinov, R. R. (2004). Dopamine transporter-dependent and -independent actions of trace amine beta-phenylethylamine. J Neurochem 91, 362-373.
Tan, J. K. M., C.; Mariño, E; Macia, L and Mackay, C R. (2017). Metabolite-Sensing G Protein-Coupled Receptors-Facilitators of Diet-Related Immune Regulation. AnnuRevImmunol 35, 371-402.
Tannase, S. G., B M; Snell, E E. (1985). Purification and Properties of a Pyridoxal 5″Phosphate-dependent Histidine Decarboxylase from Morgunellu morganii A M-15* The Journal of Biological Chemistry 260, 6738-6746.
Thurmond, R. L., Gelfand, E. W., and Dunford, P. J. (2008). The role of histamine H1 and H4 receptors in allergic inflammation: the search for new antihistamines. Nat Rev Drug Discov 7, 41-53.
Tyagi, P., Mandal, M. B., Mandal, S., Patne, S. C., and Gangopadhyay, A. N. (2009). Pouch colon associated with anorectal malformations fails to show spontaneous contractions but responds to acetylcholine and histamine in vitro. J Pediatr Surg 44, 2156-2162.
Wacker, D., Stevens, R. C., and Roth, B. L. (2017). How Ligands Illuminate GPCR Molecular Pharmacology. Cell 170, 414-427.
Williams, R. M., CDS; Burnett, J R. (2008). Phenylketonuria—An Inborn Error of Phenylalanine Metabolism. Clin Biochem Rew 29, 31-41.
Yano, J. M., Yu, K., Donaldson, G. P., Shastri, G. G., Ann, P., Ma, L., Nagler, C. R., Ismagilov, R. F., Mazmanian, S. K., and Hsiao, E. Y. (2015). Indigenous bacteria from the gut microbiota regulate host serotonin biosynthesis. Cell 161, 264-276.

The present application describes a number of examples and embodiments of the invention. Nevertheless, it must be borne in mind that various modifications of the described examples and embodiments can be developed, while not departing from the scope and the essence of the invention in principle. With this in mind, other embodiments are included in the scope of the items listed below. At that, all the numerical ranges described herein include all the sub ranges contained therein, as well as any individual values within the scope of these ranges. All publications, patents and patent applications mentioned in this description are hereby incorporated by reference.

1. A method for determining if a test compound modulates an activity of a first protein in vivo, comprising
(a) contacting said compound with a cell comprising (i) a first nucleic acid molecule which encodes a first fusion protein comprising a first protein, a cleavage site for a protease, and a protein which activates transcription of a reporter gene in said cell, (ii) a second nucleic acid molecule which encodes a second fusion protein comprising a second protein which interacts with the first protein upon activation of the first protein and a protease or a fragment thereof capable of cleaving the protease cleavage site within the first fusion protein, and (iii) a third nucleic acid molecule which comprises a reporter gene, wherein said reporter gene is a barcode sequence operably linked to an element responsive to the protein which activates its transcription, and
(b) determining the level of transcription of said barcode sequence.

2. The method of claim 1, wherein the first nucleic acid molecule, the second nucleic acid molecule and the third nucleic acid molecule are clonally expressed to enable linkage of a specific receptor to an individual barcode.

3. The method of claim 2, wherein the first nucleic acid molecule, the second nucleic acid molecule and the third nucleic acid molecule are clonally expressed through co-transfection.

4. The method of claim 2, wherein the first nucleic acid molecule, the second nucleic acid molecule and the third nucleic acid molecule are clonally expressed through stable expression

5. The method of any one of claims 1-4, wherein the barcode sequence comprises from 4 to 50 bases.

6. The method of any one of claims 1-5, further comprising
(c) concluding that the test compound activates the first protein if the level of transcription of said barcode sequence is increased in the presence of the test compound as compared to a control where no test compound is present.

7. The method of any one of claims 1-5, further comprising
(c) concluding that the test compound activates the first protein if the level of transcription of said barcode sequence is increased in the presence of the test compound as compared to an untreated cell.

8. The method of any one of claims 1-5, further comprising
(c) concluding that the test compound activates the first protein if the level of transcription of said barcode sequence is increased in the presence of the test compound as compared to a cell treated with a single dose of an agonist of the first protein in combination with an antagonist of the first protein.

9. The method of any one of claims 1-8, wherein said first protein is a transmembrane protein.

10. The method of any one of claims 1-9, wherein said first protein is a G-protein coupled receptor (GPCR).

11. The method of claim 10, wherein said GPCR is a non-olfactory GPCR.

12. The method of claim 10 or claim 11, wherein said GPCR is an orphan GPCR.

13. The method of any one of claims 10-12, wherein the GPCR is a human GPCR.

14. The method of any one of claims 1-13, wherein the protein which activates transcription of the reporter gene in said cell is tTA, a cas9 fusion protein, gal4/VP16, the estrogen receptor, the androgen receptor, the mineralocorticoid receptor, or the glucocorticoid receptor.

15. The method of claim 14, wherein the cas9 fusion protein is cas9-vp64.

16. The method of any one of claims 1-15, wherein the protein which activates transcription of the reporter gene in said cell is tTA.

17. The method of any one of claims 1-16, wherein said second protein which interacts with the first protein upon activation of the first protein is β-arrestin, a G-protein receptor kinase (GRK), or G-alpha.

18. The method of claim 17, wherein said second protein is β-arrestin.

19. The method of any one of claims 1-18, wherein said protease is a Tobacco Etch Virus nuclear inclusion A protease (TEV protease).

20. The method of any one of claims 1-19, wherein the cell is a non-adherent mammalian cell.

21. The method of claim 20, wherein the cell is Expi 293 T cell, Jurkat, Hela T4, raji, ramos, cho-s, or thp1 cells.

22. The method of any one of claim 1-21, wherein the level of transcription of said barcode sequence is determined by sequencing of cDNA.

23. The method of claim 22, wherein the cDNA is produced by reverse transcription of mRNA isolated from the cell using a polydT primer.

24. The method of any one of claims 1-23, wherein the test compound is a metabolite produced by a bacterial taxon contained within a microbiota of a subject.

25. The method of any one of claims 1-23, wherein the test compound is a metabolite produced by a bacterial strain contained within a microbiota of a subject.

26. The method of claim 24 or claim 25, wherein the microbiota is a gastrointestinal (GI) microbiota.

27. The method of claim 25 or claim 26, wherein the bacterial strain is clonally arrayed and cultured in vitro.

28. The method of any one of claims 24-27, wherein the method further comprises identifying the bacterial strain.

29. The method of claim 28, wherein the bacterial strain is identified using 16S rRNA gene sequencing or whole genome sequencing.

30. The method of any one of claims 25-29, wherein the subject is human.

31. The method of any one of claims 1-30, wherein the method is conducted in a high-throughput format, comprising:
(i) transfecting or transducing a plurality of cells separated into individual wells of a multi-well plate with the first, second and third nucleic acid molecules so that each transfected or transduced cell has a specific combination of the first protein and the barcode sequence;
(ii) mixing the transfected cells;
(iii) rearraying mixed cells into individual wells of a multi-well plate;
(iv) exposing the rearrayed cell mixtures to one or more test compounds;
(v) sequencing the barcodes, and
(vi) determining which barcode sequences are increased in the presence of the test compound(s) as compared to a control where no test compound(s) is present.

32. The method of any one of claims 1-30, wherein the method is conducted in a high-throughput format, comprising:
(i) transfecting or transducing a plurality of cells that stably express the second nucleic acid molecule (Barr-TEV) with the first and third nucleic acid molecules (the receptor and barcode);
(ii) mixing the transfected cells;
(iii) rearraying mixed cells into individual wells of a multi-well plate;
(iv) exposing the rearrayed cell mixtures to one or more test compounds;
(v) sequencing the barcodes, and
(vi) determining which barcode sequences are increased in the presence of the test compound(s) as compared to a control where no test compound(s) is present.

33. The method of claim 32, wherein the first nucleic acid molecule encodes a GPCR, the second nucleic acid molecule encodes Barr-TEV, and the third nucleic acid molecule comprises a barcode.

34. A method for high-throughput screening of microbiota metabolites capable of modulating the activity of a plurality of G-protein coupled receptors (GPCRs), the method comprising:
a) providing a plurality of non-adherent mammalian cells, wherein each cell comprises (i) a first nucleic acid molecule encoding a first fusion protein comprising a GPCR linked to the transcription factor tTA via a cleavage site for Tobacco Etch Virus nuclear inclusion A protease (TEV protease), (ii) a second nucleic acid molecule encoding a second fusion protein comprising β-arrestin and TEV protease configured to cleave the TEV protease site on the first fusion protein, and (iii) a third nucleic acid molecule comprising a barcode sequence operably linked to a promoter specifically activated by the tTA transcription factor, wherein each barcode sequence is thus specifically linked to an individual GPCR;
b) contacting the plurality of cells with one or more microbiota metabolites or one or more compounds;
c) sequencing the barcodes; and
d) determining which barcode sequence transcription levels are increased or decreased in the presence of the metabolites as compared to a control where no metabolites are present.

35. The method of claim 34, wherein said GPCR is a non-olfactory GPCR.

36. The method of claim 34 or claim 35, wherein said GPCR is an orphan GPCR.

37. The method of any one of claims 34-36, wherein the GPCR is a human GPCR.

38. The method of any one of claims 34-37, wherein the cell is Expi 293 T cell.

39. The method of any one of claims 34-38, wherein the level of transcription of said barcode sequence is determined by sequencing of cDNA.

40. The method of claim 39, wherein the cDNA is produced by reverse transcription of RNA isolated from the cell.

41. The method of any one of claims 34-40, wherein the microbiota is a gastrointestinal (GI) microbiota.

42. The method of any one of claims 34-41, wherein the method further comprises identifying a bacterial strain producing the specific metabolite which activates the specific GPCR.

43. The method of claim 42, wherein the bacterial strain is identified using 16S rRNA sequencing.

44. A method of preventing or treating monoamine oxidase inhibitor (MAOI)-induced toxicity in a subject, the method comprising administering an antibiotic effective to target a bacterial strain comprising a nucleic acid sequence encoding a phenethylamine production gene.

45. A method of preventing or treating MAOI-induced toxicity in a subject comprising administering an antibiotic effective to target Morganella spp.

46. A method of treating a disease or condition caused by decreased MAO activity in a subject, the method comprising administering an antibiotic effective to target an organism comprising a nucleic acid sequence encoding a phenethylamine production gene.

47. The method of claim 46, wherein the organism is a bacterial strain.

48. The method of any one of claim 44, 46 or 47, wherein the bacterial strain produces phenethylamine.

49. The method of claim 48, wherein the bacterial strain secretes phenethylamine.

50. The method of any one of claims 44 and 46-48, wherein the disease is Brunner syndrome.

51. The method of any one of claims 44 and 46-48, wherein the condition is autism or anti-social behavior.

52. The method of any one of claims 44 to 51, wherein the subject expresses a MAOA-L variant.

53. The method of any one of claims 44 to 52, wherein the antibiotic is effective to target Morganella morganii.

54. The method of any one of claims 44 to 53, wherein the antibiotic is cefepime, piperacillin, tazobactam, ceftazidime, cefotaxime, ceftibuten, meropenem, doripenem, ertapenem, a fluoroquinolone, or an aminoglycoside.

55. A method of treating depression in a subject comprising administering a bacterial strain comprising a nucleic acid sequence encoding a phenethylamine production gene.

56. The method of claim 55, further comprising administering an anti-depressant to the subject.

57. The method of claim 56, wherein the anti-depressant is an MAOI.

58. The method of claim 56 or claim 57, wherein the bacterial strain produces phenethylamine.

59. A method for evaluating potential toxicity of a monoamine oxidase inhibitor (MAOI) in a subject, the method comprising
a) obtaining a gastrointestinal microbiota sample from the subject, and
b) assaying the sample for the presence of a bacterial taxon or bacterial strain comprising a phenethylamine production gene.

60. The method of claim 59, wherein the amount of a phenethylamine producing enzyme exceeds a defined fraction of the amount of enzymes produced by the microbiota.

61. The method of claim 59 or claim 60, wherein the gastrointestinal microbiota sample is a fecal sample.

62. A method for evaluating potential efficacy of an MAOI in a subject, the method comprising
a) taking a fecal sample or a sample from the gastrointestinal tract of the subject, and
b) assaying for the presence of a bacterial strain comprising a nucleic acid sequence encoding a phenethylamine production gene.

63. The method of any one of claims 59 to 62, further comprising assaying for the amount of the bacterial strain present in the gastrointestinal tract.

64. The method of any one of claims 59 to 63, further comprising treating the subject with an MAOI.

65. The method of any one of claims 59 to 64, further comprising adjusting or determining the dosage of the MAOI based on the presence and/or amount of the bacterial strain present.

66. The method of any one of claims 59 to 65, wherein the bacterial strain is a bacterium of Morganella spp.

67. The method of any one of claims 59 to 65, wherein the bacterial strain is Morganella morganii.

68. A method of preventing or treating histamine-induced gastrointestinal disease in a subject, the method comprising administering one or more antibiotics effective to target a bacterial strain comprising a nucleic acid sequence encoding a histamine production gene.

69. The method of claim 68, wherein the histamine production gene is a histidine decarboxylase.

70. The method of claim 69, wherein the abundance of the histidine decarboxylase is higher in a patient with Crohn's disease as compared to a subject without inflammatory bowel disease.

71. The method of claim 69, wherein the abundance of the histidine decarboxylase is higher in a patient with ulcerative colitis as compared to a subject without inflammatory bowel disease.

72. The method of claim 68, wherein the bacterial strain is L. reuteri or a bacterium of Morganella spp.

73. The method of any one of claims 68 to 72, wherein the gastrointestinal disease is diarrhea.

74. A method of preventing or treating an allergy in a subject, the method comprising administering one or more antibiotics effective to target a bacterial strain comprising a nucleic acid sequence encoding a histamine production gene.

75. The method of claim 74, wherein the bacterial strain is L. reuteri or a bacterium of Morganella spp.

76. A method of preventing or treating asthma in a subject, the method comprising administering one or more antibiotics effective to target a bacterial strain comprising a nucleic acid sequence encoding a histamine production gene.

77. The method of claim 76, wherein the bacterial strain is L. reuteri or a bacterium of Morganella spp.

78. The method of any one of claims 68 to 77, wherein the antibiotic is cefepime, piperacillin, tazobactam, ceftazidime, cefotaxime, ceftibuten, meropenem, doripenem, ertapenem, a fluoroquinolone, or an aminoglycoside.

79. The method of any one of claims 68 to 78, further comprising administering a histidine decarboxylase inhibitor.

80. The method of claim 79, wherein the histidine decarboxylase inhibitor is rugosin D, rugosin A methyl ester, tellimagrandin II, rugosin A, pinocembrin, α-fluoromethylhistidine, brocresine, lecanoric acid, 2-hydroxy-5-carbomethoxybenzyloxyamine, and aminooxy analogs of histamine.

81. A method for evaluating potential effectiveness of an antibiotic to treat a gastrointestinal condition or disease, allergy or asthma in a subject, the method comprising
a) taking a fecal sample or a sample from the gastrointestinal tract of the subject, and
b) assaying for the presence of a bacterial strain comprising a nucleic acid sequence encoding a histamine production gene.

82. The method of claim 81, further comprising assaying for the amount of the bacterial strain present in the gastrointestinal tract.

83. The method of claim 81 or claim 82, further comprising treating the subject with one or more antibiotics.

84. The method of claim 83, wherein the antibiotic is cefepime, piperacillin, tazobactam, ceftazidime, cefotaxime, ceftibuten, meropenem, doripenem, ertapenem, a fluoroquinolone, or an aminoglycoside.

85. The method of any one of claims 81 to 84, further comprising adjusting or determining the dosage of the antibiotic based on the presence and/or amount of the bacterial strain present.

86. The method of any one of claims 81 to 85, wherein the bacterial strain is a bacterium of Morganella spp.

87. The method of any one of claims 81 to 86, wherein the bacterial strain is Morganella morganii.

88. A method of preventing or treating a disease or condition resulting from production of phenethylamine, the method comprising administering one or more antibiotics effective to target a bacterial strain producing L-phenylalanine.

89. A method of preventing or treating phenylketonuria (PKU) in a subject, the method comprising administering one or more antibiotics effective to target a bacterial strain producing L-phenylalanine.

90. A method of preventing or treating a disease or condition resulting from production of phenethylamine, the method comprising administering a Shikimate pathway inhibitor or an antagonist of aromatic L-amino acid decarboxylase.

91. A method of preventing or treating phenylketonuria (PKU) in a subject, the method comprising administering a Shikimate pathway inhibitor or an antagonist of aromatic L-amino acid decarboxylase.

92. The method of claim 90 or claim 91, wherein the antagonist is carbidopa, benserazide, methyldopa, 3′,4′,5,7-Tetrahydroxy-8-methoxyisoflavone (DFMD), 3-hydroxybenzylhydrazine, 3-amino-1-methyl-5H-pyrido[4,3-b]indole (Trp-P-2), or α-difluoromethyl DOPA.

93. The method of any one of claims 88 to 92, wherein the bacterial strain is B. thetaiotaomicron.

94. The method of claim 93, wherein the bacterial strain is a strain C34 of B. thetaiotaomicron.

95. The method of any one of claims 88 to 94, wherein the antibiotic is ampicillin, clavulanate, tazobactam, a cephamycin, ticarcillin, piperacillin, a cephalosporin, a carbapenem, clindamycin, lincomycin, chloramphenicol, a nitroimidazole, a fluoroquinolone.

96. The method of any one of claims 88 to 94, wherein the antibiotic comprises (a) a combination of ampicillin and sulbactam, (b) a combination of ticarcillin and clavulanate, or (c) a combination of piperacillin and tazobactam.

97. The method of any one of claims 44-54 and claims 68-96, further comprising administering a probiotic composition.

98. The method of claim 97, wherein the probiotic composition is administered after antibiotic administration.

99. The method of claim 97 or 98, wherein the administration of the antibiotic and the administration of the probiotic composition are repeated in a cycle.

100. A kit for evaluating potential toxicity of an MAOI in a subject, the kit comprising a nucleic acid, antibody, or other reagent capable of binding specifically to a nucleotide or protein expressed by a bacterial strain, wherein the bacterial strain comprises a nucleic acid sequence encoding a phenethylamine production gene.

101. The kit of claim 100, wherein the bacterial strain is a bacterium of the Morganella spp.

102. The kit of claim 100, wherein the bacterial strain is Morganella morganii.

103. A kit for evaluating potential effectiveness of an antibiotic to treat a gastrointestinal condition or disease, allergy or asthma in a subject, the kit comprising a nucleic acid, antibody, or other reagent capable of binding specifically to a nucleotide or protein expressed by a bacterial strain, wherein the bacterial strain comprises a nucleic acid sequence encoding a histamine production gene.

104. The kit of claim 103, wherein the bacterial strain is L. reuteri or a bacterium of Morganella spp.
